(19) United States
(12) Patent Application Publication
(10) Pub. No.: US 2023/0272056 A1
(43) Pub. Date: Aug. 31, 2023

(54) AFFINITY MATURED ANTI-LAP ANTIBODIES AND USES THEREOF

(71) Applicant: Merck Sharp & Dohme LLC, Rahway, NJ (US)

(72) Inventors: Chung-Ming Hsieh, Newton, MA (US); Michelle Castor, Jamaica Plain, MA (US); Ming-Tang Chen, Dover, MA (US); Alan C. Cheng, San Francisco, CA (US); Scott A. Hollingsworth, Redwood City, CA (US); Veronica M. Juan, Redwood City, CA (US); Madhura Shidhore, Winchester, MA (US); Song Yang, San Francisco, CA (US); Renee C.T. Moore, Medway, MA (US)

(73) Assignee: Merck Sharp & Dohme LLC, Rahway, NJ (US)

(21) Appl. No.: 17/916,664

(22) PCT Filed: Apr. 8, 2021
(86) PCT No.: PCT/US2021/026310

§ 371 Date: Oct. 3, 2022

Related U.S. Application Data
(60) Provisional application No. 63/007,707, filed on Apr. 9, 2020.

(51) Int. Cl. C07K 16/22

(52) U.S. Cl. CPC C07K 16/22; C07K 2317/565; C07K 2317/55; C07K 2317/92; C07K 2317/24

(57) ABSTRACT

Provided herein are anti-LAP antibodies (e.g., recombinant humanized, chimeric, and human anti-LAP antibodies) or antigen binding fragments thereof which have therapeutically beneficial properties, such as binding specifically to LAP-TGFβ1 on cells but not to LAP-TGFβ1 in extracellular matrix, as well as compositions including the same. Also provided are uses of these antibodies or antigen binding fragments in therapeutic applications, such as in the treatment of cancer, and diagnostic applications.

### FIELD OF THE INVENTION

The present invention relates to affinity matured anti-LAP antibodies or antigen binding fragments thereof. Another aspect of the invention relates to compositions and kits comprising the anti-LAP antibodies or antigen binding fragments. Another aspect of the invention relates to methods for treating diseases, for example cancer, by administering the antibodies or antigen binding fragments.

### RELATED APPLICATIONS

This application claims the benefit of U.S. Provisional Patent Application No. 63/007,707, filed Apr. 9, 2020, which is incorporated by reference herein in its entirety.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Mar. 22, 2021, is named 24979USPCT-SEQLIST-22MAR2021.txt and is 1,903,429 bytes in size.

### BACKGROUND

Transforming growth factor beta 1 (TGFβ1) is synthesized as a pro-protein complex, in which the mature cytokine is caged within LAP (latency associated peptide), which is the latency associated peptide of TGFβ1. The LAP-TGFβ1 complex is disulfide bonded to one of five currently known anchor proteins: glycoprotein A repetitions predominant (GARP), Leucine-rich repeat-containing protein 33 (LRRC33), latent-transforming growth factor beta-binding protein 1 (LTBP1), latent-transforming growth factor beta-binding protein 3 (LTBP3), and latent-transforming growth factor beta-binding protein 4 (LTBP4). These anchor proteins localize latent TGFβ1 in particular sites and on particular cells within the body.

GARP, also referred to as leucine-rich repeat protein 32 or LRRC32, is a transmembrane protein that anchors LAP-TGFβ1 to the surface of lymphocytes, most notably regulatory T cells. GARP is also expressed on platelets, B cells, Natural Killer (NK) cells, fibroblasts, mesenchymal stromal cells, mesenchymal stem cells, and endothelial cells and also governs LAP-TGFβ1 expression on those cell types. LRRC33 is a transmembrane protein that is reported to anchor LAP-TGFβ1 to the surface of myeloid cells, most notably macrophages, dendritic cells, and myeloid derived suppressor cells (MDSCs). LTBP1, LTBP3, and LTBP4 are secreted molecules that anchor LAP-TGFβ1 into the extracellular matrix (ECM).

Although LAP binding agents have been used in the art as tools to identify certain cell populations, little is known about LAP's relevance in disease states. Recent developments in cancer therapy have focused on harnessing a patient's immune system by, e.g., activation of exhausted immune cell populations, vaccination, and removal of immunosuppressive cell populations. Given the ongoing need for improved strategies for targeting (and diagnosing) diseases such as cancer, improved agents and methods that are useful for these purposes are desired.

### SUMMARY

An aspect of the invention provides an antibody or antigen binding fragment thereof that binds to LAP and/or a complex comprising LAP/TGFβ1. For example, the LAP is a human LAP and/or the complex comprises human LAP/TGFβ1. In various embodiments, the antibody or antigen binding fragment thereof binds LAP that comprises at least one amino acid sequence selected from the group consisting of SEQ ID NOs: 1-7 and 1562-1564. In various embodiments, the antibody or antigen binding fragment thereof binds a portion of at least one amino acid sequence selected from the group consisting of SEQ ID NOs: 1-7 and 1562-1564. In various embodiments, the antibody or antigen binding fragment binds to human LAP, cynomolgus monkey (cyno) LAP, rat LAP, and/or mouse LAP. The antibody or antigen binding fragment thereof comprises at least one amino acid sequence described in one of the Tables herein, for example, an antibody or antigen binding fragment (e.g., HCDR1, HCDR2, HCDR3, LCDR1, LCDR2, LCDR3, heavy chain variable region, light chain variable region, heavy chain, or light chain) having an amino acid sequence described in Tables 4, 6, 8, and 11-43. In various embodiments, the antibody is an isolated antibody. In various embodiments, the antibody or antigen binding fragment thereof is a monoclonal antibody. In various embodiments, the antibody or antigen binding fragment thereof is an isolated monoclonal antibody. In various embodiments, the antibody or antigen binding fragment thereof is affinity matured. In various embodiments, the antibody or antigen binding fragment thereof is an affinity matured variant of the parental 20E6 antibody or antigen binding fragment thereof (WO2020076969) comprising heavy chain complementarity determining regions (CDRs) and light chain CDRs found in Table 6. The amino acid sequences for the heavy chain variable region and the light chain variable region for the parental 20E6 antibody are listed below.

parental 20E6-VH
(SEQ ID NO: 2227)
EVQLVQSGAEVKKPGASVKVSCKASGYTFTSYWMH
WVRQAPGQGLEWMGRIDPQSGGIKYAQKFQGRATL
TVDTSTSTAYMELSRLRSDDTAVYYCARWDYGGYF
DVWGQGTLVTVSS
parental 20E6-VL
(SEQ ID NO: 2228)
DIQMTQSPSSLSASVGDRVTITCRASQDITNYLNW
YQQKPGKAVKLLIYYTSRLHSGVPSRFSGSGSGTD
YTLTISSLQPEDFATYFCQQGDTLPWTFGQGTKLE
IK

In various embodiments, the antibody or antigen binding fragment thereof is an affinity matured variant of the 20E6 antibody or antigen binding fragment thereof comprising a heavy chain variable region comprising CDR1, CDR2, and CDR3 regions comprising the amino acid sequences of SEQ ID NOs: 445, 446, and 447, respectively, and/or a light chain variable region comprising CDR1, CDR2, and CDR3 regions comprising the amino acid sequences of SEQ ID NOs: 450, 451, and 452, respectively. In various embodiments, the 20E6 antibody or antigen binding fragment thereof comprises a heavy chain variable region comprising the amino acid sequence of SEQ ID NO: 448 and/or a light chain variable region comprising the amino acid sequence of SEQ ID NO: 453. In various embodiments, the 20E6 antibody or antigen binding fragment thereof comprises a heavy chain comprising the amino acid sequence of SEQ ID NO: 449 and/or a light chain comprising the amino acid sequence of SEQ ID NO: 454.

An aspect of the invention provides an antibody or antigen binding fragment thereof comprising heavy chain CDRs and light chain CDRs found within a heavy chain variable region and a light chain variable region described in any of Tables 4, 6, 8, 11-43, and 45.

An aspect of the invention provides an antibody (e.g., an isolated antibody) or antigen binding fragment thereof which specifically binds to LAP and/or a complex comprising LAP/TGFβ1-comprising:
(a) a heavy chain variable region comprising CDR1, CDR2, and CDR3 regions comprising the amino acid sequences of SEQ ID NOs: 1285, 1286, and 1287, respectively, and/or a light chain variable region comprising CDR1, CDR2, and CDR3 regions comprising the amino acid sequences of SEQ ID NOs: 1289, 1290, and 1291, respectively;
(b) a heavy chain variable region comprising CDR1, CDR2, and CDR3 regions comprising the amino acid sequences of SEQ ID NOs: 1277, 1278, and 1279, respectively, and/or a light chain variable region comprising CDR1, CDR2, and CDR3 regions comprising the amino acid sequences of SEQ ID NOs: 1281, 1282, and 1283, respectively;
(c) a heavy chain variable region comprising CDR1, CDR2, and CDR3 regions comprising the amino acid sequences of SEQ ID NOs: 1269, 1270, and 1271, respectively, and/or a light chain variable region comprising CDR1, CDR2, and CDR3 regions comprising the amino acid sequences of SEQ ID NOs: 1273, 1274, and 1275, respectively;
(d) a heavy chain variable region comprising CDR1, CDR2, and CDR3 regions comprising the amino acid sequences of SEQ ID NOs: 1261, 1262, and 1263, respectively, and/or a light chain variable region comprising CDR1, CDR2, and CDR3 regions comprising the amino acid sequences of SEQ ID NOs: 1265, 1266, and 1267, respectively;
(e) a heavy chain variable region comprising CDR1, CDR2, and CDR3 regions comprising the amino acid sequences of SEQ ID NOs: 1253, 1254, and 1255, respectively, and/or a light chain variable region comprising CDR1, CDR2, and CDR3 regions comprising the amino acid sequences of SEQ ID NOs: 1257, 1258, and 1259, respectively;
(f) a heavy chain variable region comprising CDR1, CDR2, and CDR3 regions comprising the amino acid sequences of SEQ ID NOs: 1245, 1246, and 1247, respectively, and/or a light chain variable region comprising CDR1, CDR2, and CDR3 regions comprising the amino acid sequences of SEQ ID NOs: 1249, 1250, and 1251, respectively;
(g) a heavy chain variable region comprising CDR1, CDR2, and CDR3 regions comprising the amino acid sequences of SEQ ID NOs: 1237, 1238, and 1239, respectively, and/or a light chain variable region comprising CDR1, CDR2, and CDR3 regions comprising the amino acid sequences of SEQ ID NOs: 1241, 1242, and 1243, respectively;
(h) a heavy chain variable region comprising CDR1, CDR2, and CDR3 regions comprising the amino acid sequences of SEQ ID NOs: 1229, 1230, and 1231, respectively, and/or a light chain variable region comprising CDR1, CDR2, and CDR3 regions comprising the amino acid sequences of SEQ ID NOs: 1233, 1234, and 1235, respectively;
(i) a heavy chain variable region comprising CDR1, CDR2, and CDR3 regions comprising the amino acid sequences of SEQ ID NOs: 1221, 1222, and 1223, respectively, and/or a light chain variable region comprising CDR1, CDR2, and CDR3 regions comprising the amino acid sequences of SEQ ID NOs: 1225, 1226, and 1227, respectively;
(j) a heavy chain variable region comprising CDR1, CDR2, and CDR3 regions comprising the amino acid sequences of SEQ ID NOs: 1213, 1214, and 1215, respectively, and/or a light chain variable region comprising CDR1, CDR2, and CDR3 regions comprising the amino acid sequences of SEQ ID NOs: 1217, 1218, and 1219, respectively;
(k) a heavy chain variable region comprising CDR1, CDR2, and CDR3 regions comprising the amino acid sequences of SEQ ID NOs: 95, 96, and 97, respectively, and/or a light chain variable region comprising CDR1, CDR2, and CDR3 regions comprising the amino acid sequences of SEQ ID NOs: 100, 101, and 102, respectively;
(l) a heavy chain variable region comprising CDR1, CDR2, and CDR3 regions comprising the amino acid sequences of SEQ ID NOs: 105, 106, and 107, respectively, and/or a light chain variable region comprising CDR1, CDR2, and CDR3 regions comprising the amino acid sequences of SEQ ID NOs: 110, 111, and 112, respectively;
(m) a heavy chain variable region comprising CDR1, CDR2, and CDR3 regions comprising the amino acid sequences of SEQ ID NOs: 115, 116, and 117, respectively, and/or a light chain variable region comprising CDR1, CDR2, and CDR3 regions comprising the amino acid sequences of SEQ ID NOs: 120, 121, and 122, respectively;
(n) a heavy chain variable region comprising CDR1, CDR2, and CDR3 regions comprising the amino acid sequences of SEQ ID NOs: 125, 126, and 127, respectively, and/or a light chain variable region comprising CDR1, CDR2, and CDR3 regions comprising the amino acid sequences of SEQ ID NOs:130, 131, and 132, respectively;
(o) a heavy chain variable region comprising CDR1, CDR2, and CDR3 regions comprising the amino acid sequences of SEQ ID NOs: 135, 136, and 137, respectively, and/or a light chain variable region comprising CDR1, CDR2, and CDR3 regions comprising the amino acid sequences of SEQ ID NOs: 140, 141, and 142, respectively;
(p) a heavy chain variable region comprising CDR1, CDR2, and CDR3 regions comprising the amino acid sequences of SEQ ID NOs: 145, 146, and 147, respectively, and/or a light chain variable region comprising CDR1, CDR2, and CDR3 regions comprising the amino acid sequences of SEQ ID NOs: 150, 151, and 152, respectively;
(q) a heavy chain variable region comprising CDR1, CDR2, and CDR3 regions comprising the amino acid sequences of SEQ ID NOs: 155, 156, and 157, respectively, and/or a light chain variable region comprising CDR1, CDR2, and CDR3 regions comprising the amino acid sequences of SEQ ID NOs: 160, 161, and 162, respectively;
(r) a heavy chain variable region comprising CDR1, CDR2, and CDR3 regions comprising the amino acid sequences of SEQ ID NOs: 165, 166, and 167, respectively, and/or a light chain variable region comprising CDR1, CDR2, and CDR3 regions comprising the amino acid sequences of SEQ ID NOs: 170, 171, and 172, respectively;
(s) a heavy chain variable region comprising CDR1, CDR2, and CDR3 regions comprising the amino acid sequences of SEQ ID NOs: 175, 176, and 177, respectively, and/or a light chain variable region comprising CDR1, CDR2, and CDR3 regions comprising the amino acid sequences of SEQ ID NOs: 180, 181, and 182, respectively;
(t) a heavy chain variable region comprising CDR1, CDR2, and CDR3 regions comprising the amino acid sequences of SEQ ID NOs: 185, 186, and 187, respectively, and/or a light chain variable region comprising CDR1, CDR2, and CDR3 regions comprising the amino acid sequences of SEQ ID NOs: 190, 191, and 192, respectively;
(u) a heavy chain variable region comprising CDR1, CDR2, and CDR3 regions comprising the amino acid sequences of SEQ ID NOs: 195, 196, and 197, respectively, and/or a light chain variable region comprising CDR1, CDR2, and CDR3 regions comprising the amino acid sequences of SEQ ID NOs: 200, 201, and 202, respectively;
(v) a heavy chain variable region comprising CDR1, CDR2, and CDR3 regions comprising the amino acid sequences of SEQ ID NOs: 205, 206, and 207, respectively, and/or a light chain variable region comprising CDR1, CDR2, and CDR3 regions comprising the amino acid sequences of SEQ ID NOs: 210, 211, and 212, respectively;
(w) a heavy chain variable region comprising CDR1, CDR2, and CDR3 regions comprising the amino acid sequences of SEQ ID NOs: 215, 216, and 217, respectively, and/or a light chain variable region comprising CDR1, CDR2, and CDR3 regions comprising the amino acid sequences of SEQ ID NOs: 220, 221, and 222, respectively;
(x) a heavy chain variable region comprising CDR1, CDR2, and CDR3 regions comprising the amino acid sequences of SEQ ID NOs: 225, 226, and 227, respectively, and/or a light chain variable region comprising CDR1, CDR2, and CDR3 regions comprising the amino acid sequences of SEQ ID NOs: 230, 231, and 232, respectively;
(y) a heavy chain variable region comprising CDR1, CDR2, and CDR3 regions comprising the amino acid sequences of SEQ ID NOs: 235, 236, and 237, respectively, and/or a light chain variable region comprising CDR1, CDR2, and CDR3 regions comprising the amino acid sequences of SEQ ID NOs: 240, 241, and 242, respectively;
(z) a heavy chain variable region comprising CDR1, CDR2, and CDR3 regions comprising the amino acid sequences of SEQ ID NOs: 245, 246, and 247, respectively, and/or a light chain variable region comprising CDR1, CDR2, and CDR3 regions comprising the amino acid sequences of SEQ ID NOs: 250, 251, and 252, respectively;
(aa) a heavy chain variable region comprising CDR1, CDR2, and CDR3 regions comprising the amino acid sequences of SEQ ID NOs: 255, 256, and 257, respectively, and/or a light chain variable region comprising CDR1, CDR2, and CDR3 regions comprising the amino acid sequences of SEQ ID NOs: 260, 261, and 262, respectively;
(bb) a heavy chain variable region comprising CDR1, CDR2, and CDR3 regions comprising the amino acid sequences of SEQ ID NOs: 265, 266, and 267, respectively, and/or a light chain variable region comprising CDR1, CDR2, and CDR3 regions comprising the amino acid sequences of SEQ ID NOs: 270, 271, and 272, respectively;
(cc) a heavy chain variable region comprising CDR1, CDR2, and CDR3 regions comprising the amino acid sequences of SEQ ID NOs: 275, 276, and 277, respectively, and/or a light chain variable region comprising CDR1, CDR2, and CDR3 regions comprising the amino acid sequences of SEQ ID NOs: 280, 281, and 282, respectively;
(dd) a heavy chain variable region comprising CDR1, CDR2, and CDR3 regions comprising the amino acid sequences of SEQ ID NOs: 285, 286, and 287, respectively, and/or a light chain variable region comprising CDR1, CDR2, and CDR3 regions comprising the amino acid sequences of SEQ ID NOs: 290, 291, and 292, respectively;
(ee) a heavy chain variable region comprising CDR1, CDR2, and CDR3 regions comprising the amino acid sequences of SEQ ID NOs: 295, 296, and 297, respectively, and/or a light chain variable region comprising CDR1, CDR2, and CDR3 regions comprising the amino acid sequences of SEQ ID NOs: 300, 301, and 302, respectively;
(ff) a heavy chain variable region comprising CDR1, CDR2, and CDR3 regions comprising the amino acid sequences of SEQ ID NOs: 305, 306, and 307, respectively, and/or a light chain variable region comprising CDR1, CDR2, and CDR3 regions comprising the amino acid sequences of SEQ ID NOs: 310, 311, and 312, respectively;
(gg) a heavy chain variable region comprising CDR1, CDR2, and CDR3 regions comprising the amino acid sequences of SEQ ID NOs: 315, 316, and 317, respectively, and/or a light chain variable region comprising CDR1, CDR2, and CDR3 regions comprising the amino acid sequences of SEQ ID NOs: 320, 321, and 322, respectively;
(hh) a heavy chain variable region comprising CDR1, CDR2, and CDR3 regions comprising the amino acid sequences of SEQ ID NOs: 325, 326, and 327, respectively, and/or a light chain variable region comprising CDR1, CDR2, and CDR3 regions comprising the amino acid sequences of SEQ ID NOs: 330, 331, and 332, respectively;
(ii) a heavy chain variable region comprising CDR1, CDR2, and CDR3 regions comprising the amino acid sequences of SEQ ID NOs: 335, 336, and 337, respectively, and/or a light chain variable region comprising CDR1, CDR2, and CDR3 regions comprising the amino acid sequences of SEQ ID NOs: 340, 341, and 342, respectively;
(jj) a heavy chain variable region comprising CDR1, CDR2, and CDR3 regions comprising the amino acid sequences of SEQ ID NOs: 345, 346, and 347, respectively, and/or a light chain variable region comprising CDR1, CDR2, and CDR3 regions comprising the amino acid sequences of SEQ ID NOs: 350, 351, and 352, respectively;
(kk) a heavy chain variable region comprising CDR1, CDR2, and CDR3 regions comprising the amino acid sequences of SEQ ID NOs: 355, 356, and 357, respectively, and/or a light chain variable region comprising CDR1, CDR2, and CDR3 regions comprising the amino acid sequences of SEQ ID NOs: 360, 361, and 362, respectively;
(ll) a heavy chain variable region comprising CDR1, CDR2, and CDR3 regions comprising the amino acid sequences of SEQ ID NOs: 365, 366, and 367, respectively, and/or a light chain variable region comprising CDR1, CDR2, and CDR3 regions comprising the amino acid sequences of SEQ ID NOs: 370, 371, and 372, respectively;
(mm) a heavy chain variable region comprising CDR1, CDR2, and CDR3 regions comprising the amino acid sequences of SEQ ID NOs: 375, 376, and 377, respectively, and/or a light chain variable region comprising CDR1, CDR2, and CDR3 regions comprising the amino acid sequences of SEQ ID NOs: 380, 381, and 382, respectively;
(nn) a heavy chain variable region comprising CDR1, CDR2, and CDR3 regions comprising the amino acid sequences of SEQ ID NOs: 385, 386, and 387, respectively, and/or a light chain variable region comprising CDR1, CDR2, and CDR3 regions comprising the amino acid sequences of SEQ ID NOs: 390, 391, and 392, respectively;
(oo) a heavy chain variable region comprising CDR1, CDR2, and CDR3 regions comprising the amino acid sequences of SEQ ID NOs: 395, 396, and 397, respectively, and/or a light chain variable region comprising CDR1, CDR2, and CDR3 regions comprising the amino acid sequences of SEQ ID NOs: 400, 401, and 402, respectively;
(pp) a heavy chain variable region comprising CDR1, CDR2, and CDR3 regions comprising the amino acid sequences of SEQ ID NOs: 405, 406, and 407, respectively, and/or a light chain variable region comprising CDR1, CDR2, and CDR3 regions comprising the amino acid sequences of SEQ ID NOs: 410, 411, and 412, respectively;
(qq) a heavy chain variable region comprising CDR1, CDR2, and CDR3 regions comprising the amino acid sequences of SEQ ID NOs: 415, 416, and 417, respectively, and/or a light chain variable region comprising CDR1, CDR2, and CDR3 regions comprising the amino acid sequences of SEQ ID NOs: 420, 421, and 422, respectively;
(rr) a heavy chain variable region comprising CDR1, CDR2, and CDR3 regions comprising the amino acid sequences of SEQ ID NOs: 425, 426, and 427, respectively, and/or a light chain variable region comprising CDR1, CDR2, and CDR3 regions comprising the amino acid sequences of SEQ ID NOs: 430, 431, and 432, respectively;
(ss) a heavy chain variable region comprising CDR1, CDR2, and CDR3 regions comprising the amino acid sequences of SEQ ID NOs: 435, 436, and 437, respectively, and/or a light chain variable region comprising CDR1, CDR2, and CDR3 regions comprising the amino acid sequences of SEQ ID NOs: 440, 441, and 442, respectively;
(tt) a heavy chain variable region comprising CDR1, CDR2, and CDR3 regions comprising the amino acid sequences of SEQ ID NOs: 455, 456, and 457, respectively, and/or a light chain variable region comprising CDR1, CDR2, and CDR3 regions comprising the amino acid sequences of SEQ ID NOs: 460, 461, and 462, respectively;
(uu) a heavy chain variable region comprising CDR1, CDR2, and CDR3 regions comprising the amino acid sequences of SEQ ID NOs: 465, 466, and 467, respectively, and/or a light chain variable region comprising CDR1, CDR2, and CDR3 regions comprising the amino acid sequences of SEQ ID NOs: 470, 471, and 472, respectively;
(vv) a heavy chain variable region comprising CDR1, CDR2, and CDR3 regions comprising the amino acid sequences of SEQ ID NOs: 475, 476, and 477, respectively, and/or a light chain variable region comprising CDR1, CDR2, and CDR3 regions comprising the amino acid sequences of SEQ ID NOs: 480, 481, and 482, respectively;
(ww) a heavy chain variable region comprising CDR1, CDR2, and CDR3 regions comprising the amino acid sequences of SEQ ID NOs: 485, 486, and 487, respectively, and/or a light chain variable region comprising CDR1, CDR2, and CDR3 regions comprising the amino acid sequences of SEQ ID NOs: 490, 491, and 492, respectively;
(xx) a heavy chain variable region comprising CDR1, CDR2, and CDR3 regions comprising the amino acid sequences of SEQ ID NOs: 495, 496, and 497, respectively, and/or a light chain variable region comprising CDR1, CDR2, and CDR3 regions comprising the amino acid sequences of SEQ ID NOs: 500, 501, and 502, respectively;
(yy) a heavy chain variable region comprising CDR1, CDR2, and CDR3 regions comprising the amino acid sequences of SEQ ID NOs: 505, 506, and 507, respectively, and/or a light chain variable region comprising CDR1, CDR2, and CDR3 regions comprising the amino acid sequences of SEQ ID NOs: 510, 511, and 512, respectively;
(zz) a heavy chain variable region comprising CDR1, CDR2, and CDR3 regions comprising the amino acid sequences of SEQ ID NOs: 515, 516, and 517, respectively, and/or a light chain variable region comprising CDR1, CDR2, and CDR3 regions comprising the amino acid sequences of SEQ ID NOs: 520, 521, and 522, respectively;
(aaa) a heavy chain variable region comprising CDR1, CDR2, and CDR3 regions comprising the amino acid sequences of SEQ ID NOs: 525, 526, and 527, respectively, and/or a light chain variable region comprising CDR1, CDR2, and CDR3 regions comprising the amino acid sequences of SEQ ID NOs: 530, 531, and 532, respectively;
(bbb) a heavy chain variable region comprising CDR1, CDR2, and CDR3 regions comprising the amino acid sequences of SEQ ID NOs: 535, 536, and 537, respectively, and/or a light chain variable region comprising CDR1, CDR2, and CDR3 regions comprising the amino acid sequences of SEQ ID NOs: 540, 541, and 542, respectively;
(ccc) a heavy chain variable region comprising CDR1, CDR2, and CDR3 regions comprising the amino acid sequences of SEQ ID NOs: 545, 546, and 547, respectively, and/or a light chain variable region comprising CDR1, CDR2, and CDR3 regions comprising the amino acid sequences of SEQ ID NOs: 550, 551, and 552, respectively;
(ddd) a heavy chain variable region comprising CDR1, CDR2, and CDR3 regions comprising the amino acid sequences of SEQ ID NOs: 555, 556, and 557, respectively, and/or a light chain variable region comprising CDR1, CDR2, and CDR3 regions comprising the amino acid sequences of SEQ ID NOs: 560, 561, and 562, respectively;
(eee) a heavy chain variable region comprising CDR1, CDR2, and CDR3 regions comprising the amino acid sequences of SEQ ID NOs: 565, 566, and 567, respectively, and/or a light chain variable region comprising CDR1, CDR2, and CDR3 regions comprising the amino acid sequences of SEQ ID NOs: 570, 571, and 572, respectively;
(fff) a heavy chain variable region comprising CDR1, CDR2, and CDR3 regions comprising the amino acid sequences of SEQ ID NOs: 43, 44, and 45, respectively;
(ggg) a heavy chain variable region comprising CDR1, CDR2, and CDR3 regions comprising the amino acid sequences of SEQ ID NOs: 48, 49, and 50, respectively;
(hhh) a heavy chain variable region comprising CDR1, CDR2, and CDR3 regions comprising the amino acid sequences of SEQ ID NOs: 53, 54, and 55, respectively;
(iii) a heavy chain variable region comprising CDR1, CDR2, and CDR3 regions comprising the amino acid sequences of SEQ ID NOs: 58, 59, and 60, respectively;
(jjj) a heavy chain variable region comprising CDR1, CDR2, and CDR3 regions comprising the amino acid sequences of SEQ ID NOs: 63, 64, and 65, respectively;
(kkk) a heavy chain variable region comprising CDR1, CDR2, and CDR3 regions comprising the amino acid sequences of SEQ ID NOs: 68, 69, and 70, respectively;
(lll) a heavy chain variable region comprising CDR1, CDR2, and CDR3 regions comprising the amino acid sequences of SEQ ID NOs: 73, 74, and 75, respectively;
(mmm) a heavy chain variable region comprising CDR1, CDR2, and CDR3 regions comprising the amino acid sequences of SEQ ID NOs: 78, 79, and 80, respectively;
(nnn) a heavy chain variable region comprising CDR1, CDR2, and CDR3 regions comprising the amino acid sequences of SEQ ID NOs: 83, 84, and 85, respectively;
(ooo) a heavy chain variable region comprising CDR1, CDR2, and CDR3 regions comprising the amino acid sequences of SEQ ID NOs: 88, 89, and 90, respectively;
(ppp) a light chain variable region comprising CDR1, CDR2, and CDR3 regions comprising the amino acid sequences of SEQ ID NOs: 8, 9, and 10, respectively;
(qqq) a light chain variable region comprising CDR1, CDR2, and CDR3 regions comprising the amino acid sequences of SEQ ID NOs: 13, 14, and 15, respectively;
(rrr) a heavy chain variable region comprising CDR1, CDR2, and CDR3 regions comprising the amino acid sequences of SEQ ID NOs: 18, 19, and 20, respectively;
(sss) a light chain variable region comprising CDR1, CDR2, and CDR3 regions comprising the amino acid sequences of SEQ ID NOs: 23, 24, and 25, respectively;
(ttt) a light chain variable region comprising CDR1, CDR2, and CDR3 regions comprising the amino acid sequences of SEQ ID NOs: 28, 29, and 30, respectively;
(uuu) a heavy chain variable region comprising CDR1, CDR2, and CDR3 regions comprising the amino acid sequences of SEQ ID NOs: 33, 34, and 35, respectively;
(vvv) a heavy chain variable region comprising CDR1, CDR2, and CDR3 regions comprising the amino acid sequences of SEQ ID NOs: 38, 39, and 40, respectively; or
(zzz) a heavy chain variable region comprising CDR1, CDR2, and CDR3 regions comprising CDR1, CDR2, and CDR3 amino acid sequences selected from the group of sequences set forth in Table 42, Table 43, and/or Table 45B, respectively, and/or a light chain variable region comprising CDR1, CDR2, and CDR3 regions comprising CDR1, CDR2, and CDR3 amino acid sequences selected from the group of sequences set forth in Table 42, Table 43, and/or Table 45B, respectively. For example, the CDR1, CDR2, and CDR3 regions in the heavy chain variable region and the light chain variable region comprising CDR1, CDR2, and CDR3 amino acid sequences selected from the group of sequences set forth in SEQ ID NOs: 1565-2212.

In various embodiments, the antibody or antigen binding fragment thereof comprises a heavy chain variable region comprising CDR1, CDR2, and CDR3 regions comprising the amino acid sequences of SEQ ID NOs: 1292, 1293, and 1294, respectively, and/or a light chain variable region comprising CDR1, CDR2, and CDR3 regions comprising the amino acid sequences of SEQ ID NOs: 1297, 1298, and 1299, respectively.

In various embodiments, the antibody or antigen binding fragment thereof comprises a heavy chain variable region comprising CDR1, CDR2, and CDR3 regions comprising the amino acid sequences of SEQ ID NOs: 1302, 1303, and 1304, respectively, and/or a light chain variable region comprising CDR1, CDR2, and CDR3 regions comprising the amino acid sequences of SEQ ID NOs: 1307, 1308, and 1309, respectively.

In various embodiments, the antibody or antigen binding fragment thereof comprises a heavy chain variable region comprising CDR1, CDR2, and CDR3 regions comprising the amino acid sequences of SEQ ID NOs: 1312, 1313, and 1314, respectively, and/or a light chain variable region comprising CDR1, CDR2, and CDR3 regions comprising the amino acid sequences of SEQ ID NOs: 1317, 1318, and 1319, respectively.

In various embodiments, the antibody or antigen binding fragment thereof comprises a heavy chain variable region comprising CDR1, CDR2, and CDR3 regions comprising the amino acid sequences of SEQ ID NOs: 1322, 1323, and 1324, respectively, and/or a light chain variable region comprising CDR1, CDR2, and CDR3 regions comprising the amino acid sequences of SEQ ID NOs: 1327, 1328, and 1329, respectively.

In various embodiments, the antibody or antigen binding fragment thereof comprises a heavy chain variable region comprising CDR1, CDR2, and CDR3 regions comprising the amino acid sequences of SEQ ID NOs: 1332, 1333, and 1334, respectively, and/or a light chain variable region comprising CDR1, CDR2, and CDR3 regions comprising the amino acid sequences of SEQ ID NOs: 1337, 1338, and 1339, respectively.

In various embodiments, the antibody or antigen binding fragment thereof comprises a heavy chain variable region comprising CDR1, CDR2, and CDR3 regions comprising the amino acid sequences of SEQ ID NOs: 1342, 1343, and 1344, and/or a light chain variable region comprising CDR1, CDR2, and CDR3 regions comprising the amino acid sequences of SEQ ID NOs: 1347, 1348, and 1349, respectively.

In various embodiments, the antibody or antigen binding fragment thereof comprises a heavy chain variable region comprising CDR1, CDR2, and CDR3 regions comprising the amino acid sequences of SEQ ID NOs: 1352, 1353, and 1354, respectively, and/or a light chain variable region comprising CDR1, CDR2, and CDR3 regions comprising the amino acid sequences of SEQ ID NOs: 1357, 1358, and 1359, respectively.

In various embodiments, the antibody or antigen binding fragment thereof comprises a heavy chain variable region comprising CDR1, CDR2, and CDR3 regions comprising the amino acid sequences of SEQ ID NOs: 1362, 1363, and 1364, respectively, and/or a light chain variable region comprising CDR1, CDR2, and CDR3 regions comprising the amino acid sequences of SEQ ID NOs: 1367, 1368, and 1369, respectively.

In various embodiments, the antibody or antigen binding fragment thereof comprises a heavy chain variable region comprising CDR1, CDR2, and CDR3 regions comprising the amino acid sequences of SEQ ID NOs: 1372, 1373, and 1374, respectively, and/or a light chain variable region comprising CDR1, CDR2, and CDR3 regions comprising the amino acid sequences of SEQ ID NOs: 1377, 1378, and 1379, respectively.

In various embodiments, the antibody or antigen binding fragment thereof comprises a heavy chain variable region comprising CDR1, CDR2, and CDR3 regions comprising the amino acid sequences of SEQ ID NOs: 1382, 1383, and 1384, respectively, and/or a light chain variable region comprising CDR1, CDR2, and CDR3 regions comprising the amino acid sequences of SEQ ID NOs: 1387, 1388, and 1389, respectively.

In various embodiments, the antibody or antigen binding fragment thereof comprises a heavy chain variable region comprising CDR1, CDR2, and CDR3 regions comprising the amino acid sequences of SEQ ID NOs: 1392, 1393, and 1394, respectively, and/or a light chain variable region comprising CDR1, CDR2, and CDR3 regions comprising the amino acid sequences of SEQ ID NOs: 1397, 1398, and 1399, respectively.

In various embodiments, the antibody or antigen binding fragment thereof comprises a heavy chain variable region comprising CDR1, CDR2, and CDR3 regions comprising the amino acid sequences of SEQ ID NOs: 1402, 1403, and 1404, respectively, and/or a light chain variable region comprising CDR1, CDR2, and CDR3 regions comprising the amino acid sequences of SEQ ID NOs: 1407, 1408, and 1409, respectively.

In various embodiments, the antibody or antigen binding fragment thereof comprises a heavy chain variable region comprising CDR1, CDR2, and CDR3 regions comprising the amino acid sequences of SEQ ID NOs: 1412, 1413, and 1414, respectively, and/or a light chain variable region comprising CDR1, CDR2, and CDR3 regions comprising the amino acid sequences of SEQ ID NOs: 1417, 1418, and 1419, respectively.

In various embodiments, the antibody or antigen binding fragment thereof comprises a heavy chain variable region comprising CDR1, CDR2, and CDR3 regions comprising the amino acid sequences of SEQ ID NOs: 1422, 1423, and 1424, respectively, and/or a light chain variable region comprising CDR1, CDR2, and CDR3 regions comprising the amino acid sequences of SEQ ID NOs: 1427, 1428, and 1429, respectively.

In various embodiments, the antibody or antigen binding fragment thereof comprises a heavy chain variable region comprising CDR1, CDR2, and CDR3 regions comprising the amino acid sequences of SEQ ID NOs: 1432, 1433, and 1434, respectively, and/or a light chain variable region comprising CDR1, CDR2, and CDR3 regions comprising the amino acid sequences of SEQ ID NOs: 1437, 1438, and 1439, respectively.

In various embodiments, the antibody or antigen binding fragment thereof comprises a heavy chain variable region comprising CDR1, CDR2, and CDR3 regions comprising the amino acid sequences of SEQ ID NOs: 1442, 1443, and 1444, and/or a light chain variable region comprising CDR1, CDR2, and CDR3 regions comprising the amino acid sequences of SEQ ID NOs: 1447, 1448, and 1449, respectively.

In various embodiments, the antibody or antigen binding fragment thereof comprises a heavy chain variable region comprising CDR1, CDR2, and CDR3 regions comprising the amino acid sequences of SEQ ID NOs: 1452, 1453, and 1454, respectively, and/or a light chain variable region comprising CDR1, CDR2, and CDR3 regions comprising the amino acid sequences of SEQ ID NOs: 1457, 1458, and 1459, respectively.

In various embodiments, the antibody or antigen binding fragment thereof comprises a heavy chain variable region comprising CDR1, CDR2, and CDR3 regions comprising the amino acid sequences of SEQ ID NOs: 1462, 1463, and 1464, respectively, and/or a light chain variable region comprising CDR1, CDR2, and CDR3 regions comprising the amino acid sequences of SEQ ID NOs: 1467, 1468, and 1469, respectively.

In various embodiments, the antibody or antigen binding fragment thereof comprises a heavy chain variable region comprising CDR1, CDR2, and CDR3 regions comprising the amino acid sequences of SEQ ID NOs: 1472, 1473, and 1474, respectively, and/or a light chain variable region comprising CDR1, CDR2, and CDR3 regions comprising the amino acid sequences of SEQ ID NOs: 1477, 1478, and 1479, respectively.

In various embodiments, the antibody or antigen binding fragment thereof comprises a heavy chain variable region comprising CDR1, CDR2, and CDR3 regions comprising the amino acid sequences of SEQ ID NOs: 1482, 1483, and 1484, respectively, and/or a light chain variable region comprising CDR1, CDR2, and CDR3 regions comprising the amino acid sequences of SEQ ID NOs: 1487, 1488, and 1489, respectively.

In various embodiments, the antibody or antigen binding fragment thereof comprises a heavy chain variable region comprising CDR1, CDR2, and CDR3 regions comprising the amino acid sequences of SEQ ID NOs: 1492, 1493, and 1494, respectively, and/or a light chain variable region comprising CDR1, CDR2, and CDR3 regions comprising the amino acid sequences of SEQ ID NOs: 1497, 1498, and 1499, respectively.

In various embodiments, the antibody or antigen binding fragment thereof comprises a heavy chain variable region comprising CDR1, CDR2, and CDR3 regions comprising the amino acid sequences of SEQ ID NOs: 1502, 1503, and 1504, respectively, and/or a light chain variable region comprising CDR1, CDR2, and CDR3 regions comprising the amino acid sequences of SEQ ID NOs: 1507, 1508, and 1509, respectively.

In various embodiments, the antibody or antigen binding fragment thereof comprises a heavy chain variable region comprising CDR1, CDR2, and CDR3 regions comprising the amino acid sequences of SEQ ID NOs: 1512, 1513, and 1514, respectively, and/or a light chain variable region comprising CDR1, CDR2, and CDR3 regions comprising the amino acid sequences of SEQ ID NOs: 1517, 1518, and 1519, respectively.

In various embodiments, the antibody or antigen binding fragment thereof comprises a heavy chain variable region comprising CDR1, CDR2, and CDR3 regions comprising the amino acid sequences of SEQ ID NOs: 1522, 1523, and 1524, respectively, and/or a light chain variable region comprising CDR1, CDR2, and CDR3 regions comprising the amino acid sequences of SEQ ID NOs: 1527, 1528, and 1529, respectively.

In various embodiments, the antibody or antigen binding fragment thereof comprises a heavy chain variable region comprising CDR1, CDR2, and CDR3 regions comprising the amino acid sequences of SEQ ID NOs: 1532, 1533, and 1534, respectively, and/or a light chain variable region comprising CDR1, CDR2, and CDR3 regions comprising the amino acid sequences of SEQ ID NOs: 1537, 1538, and 1539, respectively.

In various embodiments, the antibody or antigen binding fragment thereof comprises a heavy chain variable region comprising CDR1, CDR2, and CDR3 regions comprising the amino acid sequences of SEQ ID NOs: 1542, 1543, and 1544, and/or a light chain variable region comprising CDR1, CDR2, and CDR3 regions comprising the amino acid sequences of SEQ ID NOs: 1547, 1548, and 1549, respectively.

In various embodiments, the antibody or antigen binding fragment thereof comprises a heavy chain variable region comprising CDR1, CDR2, and CDR3 regions comprising the amino acid sequences of SEQ ID NOs: 1552, 1553, and 1554, respectively, and/or a light chain variable region comprising CDR1, CDR2, and CDR3 regions comprising the amino acid sequences of SEQ ID NOs: 1557, 1558, and 1559, respectively.

In various embodiments, the antibody or antigen binding fragment thereof comprises a heavy chain variable region comprising CDR1, CDR2, and CDR3 regions comprising the amino acid sequences of SEQ ID NOs: 1565, 1566, and 1567, respectively, and/or a light chain variable region comprising CDR1, CDR2, and CDR3 regions comprising the amino acid sequences of SEQ ID NOs: 1568, 1569, and 1570, respectively.

In various embodiments, the antibody or antigen binding fragment thereof comprises a heavy chain variable region comprising CDR1, CDR2, and CDR3 regions comprising the amino acid sequences of SEQ ID NOs: 1571, 1572, and 1573, respectively, and/or a light chain variable region comprising CDR1, CDR2, and CDR3 regions comprising the amino acid sequences of SEQ ID NOs: 1574, 1575, and 1576, respectively.

In various embodiments, the antibody or antigen binding fragment thereof comprises a heavy chain variable region comprising CDR1, CDR2, and CDR3 regions comprising the amino acid sequences of SEQ ID NOs: 1577, 1578, and 1579, respectively, and/or a light chain variable region comprising CDR1, CDR2, and CDR3 regions comprising the amino acid sequences of SEQ ID NOs: 1580, 1581, and 1582, respectively.

In various embodiments, the antibody or antigen binding fragment thereof comprises a heavy chain variable region comprising CDR1, CDR2, and CDR3 regions comprising the amino acid sequences of SEQ ID NOs: 1583, 1584, and 1585, respectively, and/or a light chain variable region comprising CDR1, CDR2, and CDR3 regions comprising the amino acid sequences of SEQ ID NOs: 1586, 1587, and 1588, respectively.

In various embodiments, the antibody or antigen binding fragment thereof comprises a heavy chain variable region comprising CDR1, CDR2, and CDR3 regions comprising the amino acid sequences of SEQ ID NOs: 1589, 1590, and 1591, respectively, and/or a light chain variable region comprising CDR1, CDR2, and CDR3 regions comprising the amino acid sequences of SEQ ID NOs: 1592, 1593, and 1594, respectively.

In various embodiments, the antibody or antigen binding fragment thereof comprises a heavy chain variable region comprising CDR1, CDR2, and CDR3 regions comprising the amino acid sequences of SEQ ID NOs: 1595, 1596, and 1597, respectively, and/or a light chain variable region comprising CDR1, CDR2, and CDR3 regions comprising the amino acid sequences of SEQ ID NOs: 1598, 1599, and 1600, respectively.

In various embodiments, the antibody or antigen binding fragment thereof comprises a heavy chain variable region comprising CDR1, CDR2, and CDR3 regions comprising the amino acid sequences of SEQ ID NOs: 1601, 1602, and 1603, respectively, and/or a light chain variable region comprising CDR1, CDR2, and CDR3 regions comprising the amino acid sequences of SEQ ID NOs: 1604, 1605, and 1606, respectively.

In various embodiments, the antibody or antigen binding fragment thereof comprises a heavy chain variable region comprising CDR1, CDR2, and CDR3 regions comprising the amino acid sequences of SEQ ID NOs: 1607, 1608, and 1609, respectively, and/or a light chain variable region comprising CDR1, CDR2, and CDR3 regions comprising the amino acid sequences of SEQ ID NOs: 1610, 1611, and 1612, respectively.

In various embodiments, the antibody or antigen binding fragment thereof comprises a heavy chain variable region comprising CDR1, CDR2, and CDR3 regions comprising the amino acid sequences of SEQ ID NOs: 1613, 1614, and 1615, respectively, and/or a light chain variable region comprising CDR1, CDR2, and CDR3 regions comprising the amino acid sequences of SEQ ID NOs: 1616, 1617, and 1618, respectively.

In various embodiments, the antibody or antigen binding fragment thereof comprises a heavy chain variable region comprising CDR1, CDR2, and CDR3 regions comprising the amino acid sequences of SEQ ID NOs: 1619, 1620, and 1621, respectively, and/or a light chain variable region comprising CDR1, CDR2, and CDR3 regions comprising the amino acid sequences of SEQ ID NOs: 1622, 1623, and 1624, respectively.

In various embodiments, the antibody or antigen binding fragment thereof comprises a heavy chain variable region comprising CDR1, CDR2, and CDR3 regions comprising the amino acid sequences of SEQ ID NOs: 1625, 1626, and 1627, respectively, and/or a light chain variable region comprising CDR1, CDR2, and CDR3 regions comprising the amino acid sequences of SEQ ID NOs: 1628, 1629, and 1630, respectively.

In various embodiments, the antibody or antigen binding fragment thereof comprises a heavy chain variable region comprising CDR1, CDR2, and CDR3 regions comprising the amino acid sequences of SEQ ID NOs: 1631, 1632, and 1633, respectively, and/or a light chain variable region comprising CDR1, CDR2, and CDR3 regions comprising the amino acid sequences of SEQ ID NOs: 1634, 1635, and 1636, respectively.

In various embodiments, the antibody or antigen binding fragment thereof comprises a heavy chain variable region comprising CDR1, CDR2, and CDR3 regions comprising the amino acid sequences of SEQ ID NOs: 1637, 1638, and 1639, respectively, and/or a light chain variable region comprising CDR1, CDR2, and CDR3 regions comprising the amino acid sequences of SEQ ID NOs: 1640, 1641, and 1642, respectively.

In various embodiments, the antibody or antigen binding fragment thereof comprises a heavy chain variable region comprising CDR1, CDR2, and CDR3 regions comprising the amino acid sequences of SEQ ID NOs: 1643, 1644, and 1645, respectively, and/or a light chain variable region comprising CDR1, CDR2, and CDR3 regions comprising the amino acid sequences of SEQ ID NOs: 1646, 1647, and 1648, respectively.

In various embodiments, the antibody or antigen binding fragment thereof comprises a heavy chain variable region comprising CDR1, CDR2, and CDR3 regions comprising the amino acid sequences of SEQ ID NOs: 1649, 1650, and 1651, respectively, and/or a light chain variable region comprising CDR1, CDR2, and CDR3 regions comprising the amino acid sequences of SEQ ID NOs: 1652, 1653, and 1654, respectively.

In various embodiments, the antibody or antigen binding fragment thereof comprises a heavy chain variable region comprising CDR1, CDR2, and CDR3 regions comprising the amino acid sequences of SEQ ID NOs: 1655, 1656, and 1657, respectively, and/or a light chain variable region comprising CDR1, CDR2, and CDR3 regions comprising the amino acid sequences of SEQ ID NOs: 1658, 1659, and 1660, respectively.

In various embodiments, the antibody or antigen binding fragment thereof comprises a heavy chain variable region comprising CDR1, CDR2, and CDR3 regions comprising the amino acid sequences of SEQ ID NOs: 1661, 1662, and 1663, respectively, and/or a light chain variable region comprising CDR1, CDR2, and CDR3 regions comprising the amino acid sequences of SEQ ID NOs: 1664, 1665, and 1666, respectively.

In various embodiments, the antibody or antigen binding fragment thereof comprises a heavy chain variable region comprising CDR1, CDR2, and CDR3 regions comprising the amino acid sequences of SEQ ID NOs: 1667, 1668, and 1669, respectively, and/or a light chain variable region comprising CDR1, CDR2, and CDR3 regions comprising the amino acid sequences of SEQ ID NOs: 1670, 1671, and 1672, respectively.

In various embodiments, the antibody or antigen binding fragment thereof comprises a heavy chain variable region comprising CDR1, CDR2, and CDR3 regions comprising the amino acid sequences of SEQ ID NOs: 1673, 1674, and 1675, respectively, and/or a light chain variable region comprising CDR1, CDR2, and CDR3 regions comprising the amino acid sequences of SEQ ID NOs: 1676, 1677, and 1678, respectively.

In various embodiments, the antibody or antigen binding fragment thereof comprises a heavy chain variable region comprising CDR1, CDR2, and CDR3 regions comprising the amino acid sequences of SEQ ID NOs: 1679, 1680, and 1681, respectively, and/or a light chain variable region comprising CDR1, CDR2, and CDR3 regions comprising the amino acid sequences of SEQ ID NOs: 1682, 1683, and 1684, respectively.

In various embodiments, the antibody or antigen binding fragment thereof comprises a heavy chain variable region comprising CDR1, CDR2, and CDR3 regions comprising the amino acid sequences of SEQ ID NOs: 1685, 1686, and 1687, respectively, and/or a light chain variable region comprising CDR1, CDR2, and CDR3 regions comprising the amino acid sequences of SEQ ID NOs: 1688, 1689, and 1690, respectively.

In various embodiments, the antibody or antigen binding fragment thereof comprises a heavy chain variable region comprising CDR1, CDR2, and CDR3 regions comprising the amino acid sequences of SEQ ID NOs: 1691, 1692, and 1693, respectively, and/or a light chain variable region comprising CDR1, CDR2, and CDR3 regions comprising the amino acid sequences of SEQ ID NOs: 1694, 1695, and 1696, respectively.

In various embodiments, the antibody or antigen binding fragment thereof comprises a heavy chain variable region comprising CDR1, CDR2, and CDR3 regions comprising the amino acid sequences of SEQ ID NOs: 1697, 1698, and 1699, respectively, and/or a light chain variable region comprising CDR1, CDR2, and CDR3 regions comprising the amino acid sequences of SEQ ID NOs: 1700, 1701, and 1702, respectively.

In various embodiments, the antibody or antigen binding fragment thereof comprises a heavy chain variable region comprising CDR1, CDR2, and CDR3 regions comprising the amino acid sequences of SEQ ID NOs: 1703, 1704, and 1705, respectively, and/or a light chain variable region comprising CDR1, CDR2, and CDR3 regions comprising the amino acid sequences of SEQ ID NOs: 1706, 1707, and 1708, respectively.

In various embodiments, the antibody or antigen binding fragment thereof comprises a heavy chain variable region comprising CDR1, CDR2, and CDR3 regions comprising the amino acid sequences of SEQ ID NOs: 1709, 1710, and 1711, respectively, and/or a light chain variable region comprising CDR1, CDR2, and CDR3 regions comprising the amino acid sequences of SEQ ID NOs: 1712, 1713, and 1714, respectively.

In various embodiments, the antibody or antigen binding fragment thereof comprises a heavy chain variable region comprising CDR1, CDR2, and CDR3 regions comprising the amino acid sequences of SEQ ID NOs: 1715, 1716, and 1717, respectively, and/or a light chain variable region comprising CDR1, CDR2, and CDR3 regions comprising the amino acid sequences of SEQ ID NOs: 1718, 1719, and 1720, respectively.

In various embodiments, the antibody or antigen binding fragment thereof comprises a heavy chain variable region comprising CDR1, CDR2, and CDR3 regions comprising the amino acid sequences of SEQ ID NOs: 1721, 1722, and 1723, respectively, and/or a light chain variable region comprising CDR1, CDR2, and CDR3 regions comprising the amino acid sequences of SEQ ID NOs: 1724, 1725, and 1726, respectively.

In various embodiments, the antibody or antigen binding fragment thereof comprises a heavy chain variable region comprising CDR1, CDR2, and CDR3 regions comprising the amino acid sequences of SEQ ID NOs: 1727, 1728, and 1729, respectively, and/or a light chain variable region comprising CDR1, CDR2, and CDR3 regions comprising the amino acid sequences of SEQ ID NOs: 1730, 1731, and 1732, respectively.

In various embodiments, the antibody or antigen binding fragment thereof comprises a heavy chain variable region comprising CDR1, CDR2, and CDR3 regions comprising the amino acid sequences of SEQ ID NOs: 1733, 1734, and 1735, respectively, and/or a light chain variable region comprising CDR1, CDR2, and CDR3 regions comprising the amino acid sequences of SEQ ID NOs: 1736, 1737, and 1738, respectively.

In various embodiments, the antibody or antigen binding fragment thereof comprises a heavy chain variable region comprising CDR1, CDR2, and CDR3 regions comprising the amino acid sequences of SEQ ID NOs: 1739, 1740, and 1741, respectively, and/or a light chain variable region comprising CDR1, CDR2, and CDR3 regions comprising the amino acid sequences of SEQ ID NOs: 1742, 1743, and 1744, respectively.

In various embodiments, the antibody or antigen binding fragment thereof comprises a heavy chain variable region comprising CDR1, CDR2, and CDR3 regions comprising the amino acid sequences of SEQ ID NOs: 1745, 1746, and 1747, respectively, and/or a light chain variable region comprising CDR1, CDR2, and CDR3 regions comprising the amino acid sequences of SEQ ID NOs: 1748, 1749, and 1750, respectively.

In various embodiments, the antibody or antigen binding fragment thereof comprises a heavy chain variable region comprising CDR1, CDR2, and CDR3 regions comprising the amino acid sequences of SEQ ID NOs: 1751, 1752, and 1753, respectively, and/or a light chain variable region comprising CDR1, CDR2, and CDR3 regions comprising the amino acid sequences of SEQ ID NOs: 1754, 1755, and 1756, respectively.

In various embodiments, the antibody or antigen binding fragment thereof comprises a heavy chain variable region comprising CDR1, CDR2, and CDR3 regions comprising the amino acid sequences of SEQ ID NOs: 1757, 1758, and 1759, respectively, and/or a light chain variable region comprising CDR1, CDR2, and CDR3 regions comprising the amino acid sequences of SEQ ID NOs: 1760, 1761, and 1762, respectively.

In various embodiments, the antibody or antigen binding fragment thereof comprises a heavy chain variable region comprising CDR1, CDR2, and CDR3 regions comprising the amino acid sequences of SEQ ID NOs: 1763, 1764, and 1765, respectively, and/or a light chain variable region comprising CDR1, CDR2, and CDR3 regions comprising the amino acid sequences of SEQ ID NOs: 1766, 1767, and 1768, respectively.

In various embodiments, the antibody or antigen binding fragment thereof comprises a heavy chain variable region comprising CDR1, CDR2, and CDR3 regions comprising the amino acid sequences of SEQ ID NOs: 1769, 1770, and 1771, respectively, and/or a light chain variable region comprising CDR1, CDR2, and CDR3 regions comprising the amino acid sequences of SEQ ID NOs: 1772, 1773, and 1774, respectively.

In various embodiments, the antibody or antigen binding fragment thereof comprises a heavy chain variable region comprising CDR1, CDR2, and CDR3 regions comprising the amino acid sequences of SEQ ID NOs: 1775, 1776, and 1777, respectively, and/or a light chain variable region comprising CDR1, CDR2, and CDR3 regions comprising the amino acid sequences of SEQ ID NOs: 1778, 1779, and 1780, respectively.

In various embodiments, the antibody or antigen binding fragment thereof comprises a heavy chain variable region comprising CDR1, CDR2, and CDR3 regions comprising the amino acid sequences of SEQ ID NOs: 1781, 1782, and 1783, respectively, and/or a light chain variable region comprising CDR1, CDR2, and CDR3 regions comprising the amino acid sequences of SEQ ID NOs: 1784, 1785, and 1786, respectively.

In various embodiments, the antibody or antigen binding fragment thereof comprises a heavy chain variable region comprising CDR1, CDR2, and CDR3 regions comprising the amino acid sequences of SEQ ID NOs: 1787, 1788, and 1789, respectively, and/or a light chain variable region comprising CDR1, CDR2, and CDR3 regions comprising the amino acid sequences of SEQ ID NOs: 1790, 1791, and 1792, respectively.

In various embodiments, the antibody or antigen binding fragment thereof comprises a heavy chain variable region comprising CDR1, CDR2, and CDR3 regions comprising the amino acid sequences of SEQ ID NOs: 1793, 1794, and 1795, respectively, and/or a light chain variable region comprising CDR1, CDR2, and CDR3 regions comprising the amino acid sequences of SEQ ID NOs: 1796, 1797, and 1798, respectively.

In various embodiments, the antibody or antigen binding fragment thereof comprises a heavy chain variable region comprising CDR1, CDR2, and CDR3 regions comprising the amino acid sequences of SEQ ID NOs: 1799, 1800, and 1801, respectively, and/or a light chain variable region comprising CDR1, CDR2, and CDR3 regions comprising the amino acid sequences of SEQ ID NOs: 1802, 1803, and 1804, respectively.

In various embodiments, the antibody or antigen binding fragment thereof comprises a heavy chain variable region comprising CDR1, CDR2, and CDR3 regions comprising the amino acid sequences of SEQ ID NOs: 1805, 1806, and 1807, respectively, and/or a light chain variable region comprising CDR1, CDR2, and CDR3 regions comprising the amino acid sequences of SEQ ID NOs: 1808, 1809, and 1810, respectively.

In various embodiments, the antibody or antigen binding fragment thereof comprises a heavy chain variable region comprising CDR1, CDR2, and CDR3 regions comprising the amino acid sequences of SEQ ID NOs: 1811, 1812, and 1813, respectively, and/or a light chain variable region comprising CDR1, CDR2, and CDR3 regions comprising the amino acid sequences of SEQ ID NOs: 1814, 1815, and 1816, respectively.

In various embodiments, the antibody or antigen binding fragment thereof comprises a heavy chain variable region comprising CDR1, CDR2, and CDR3 regions comprising the amino acid sequences of SEQ ID NOs: 1817, 1818, and 1819, respectively, and/or a light chain variable region comprising CDR1, CDR2, and CDR3 regions comprising the amino acid sequences of SEQ ID NOs: 1820, 1821, and 1822, respectively.

In various embodiments, the antibody or antigen binding fragment thereof comprises a heavy chain variable region comprising CDR1, CDR2, and CDR3 regions comprising the amino acid sequences of SEQ ID NOs: 1823, 1824, and 1825, respectively, and/or a light chain variable region comprising CDR1, CDR2, and CDR3 regions comprising the amino acid sequences of SEQ ID NOs: 1826, 1827, and 1828, respectively.

In various embodiments, the antibody or antigen binding fragment thereof comprises a heavy chain variable region comprising CDR1, CDR2, and CDR3 regions comprising the amino acid sequences of SEQ ID NOs: 1829, 1830, and 1831, respectively, and/or a light chain variable region comprising CDR1, CDR2, and CDR3 regions comprising the amino acid sequences of SEQ ID NOs: 1832, 1833, and 1834, respectively.

In various embodiments, the antibody or antigen binding fragment thereof comprises a heavy chain variable region comprising CDR1, CDR2, and CDR3 regions comprising the amino acid sequences of SEQ ID NOs: 1835, 1836, and 1837, respectively, and/or a light chain variable region comprising CDR1, CDR2, and CDR3 regions comprising the amino acid sequences of SEQ ID NOs: 1838, 1839, and 1840, respectively.

In various embodiments, the antibody or antigen binding fragment thereof comprises a heavy chain variable region comprising CDR1, CDR2, and CDR3 regions comprising the amino acid sequences of SEQ ID NOs: 1841, 1842, and 1843, respectively, and/or a light chain variable region comprising CDR1, CDR2, and CDR3 regions comprising the amino acid sequences of SEQ ID NOs: 1844, 1845, and 1846, respectively.

In various embodiments, the antibody or antigen binding fragment thereof comprises a heavy chain variable region comprising CDR1, CDR2, and CDR3 regions comprising the amino acid sequences of SEQ ID NOs: 1847, 1848, and 1849, respectively, and/or a light chain variable region comprising CDR1, CDR2, and CDR3 regions comprising the amino acid sequences of SEQ ID NOs: 1850, 1851, and 1852, respectively.

In various embodiments, the antibody or antigen binding fragment thereof comprises a heavy chain variable region comprising CDR1, CDR2, and CDR3 regions comprising the amino acid sequences of SEQ ID NOs: 1853, 1854, and 1855, respectively, and/or a light chain variable region comprising CDR1, CDR2, and CDR3 regions comprising the amino acid sequences of SEQ ID NOs: 1856, 1857, and 1858, respectively.

In various embodiments, the antibody or antigen binding fragment thereof comprises a heavy chain variable region comprising CDR1, CDR2, and CDR3 regions comprising the amino acid sequences of SEQ ID NOs: 1859, 1860, and 1861, respectively, and/or a light chain variable region comprising CDR1, CDR2, and CDR3 regions comprising the amino acid sequences of SEQ ID NOs: 1862, 1863, and 1864, respectively.

In various embodiments, the antibody or antigen binding fragment thereof comprises a heavy chain variable region comprising CDR1, CDR2, and CDR3 regions comprising the amino acid sequences of SEQ ID NOs: 1865, 1866, and 1867, respectively, and/or a light chain variable region comprising CDR1, CDR2, and CDR3 regions comprising the amino acid sequences of SEQ ID NOs: 1868, 1869, and 1870, respectively.

In various embodiments, the antibody or antigen binding fragment thereof comprises a heavy chain variable region comprising CDR1, CDR2, and CDR3 regions comprising the amino acid sequences of SEQ ID NOs: 1871, 1872, and 1873, respectively, and/or a light chain variable region comprising CDR1, CDR2, and CDR3 regions comprising the amino acid sequences of SEQ ID NOs: 1874, 1875, and 1876, respectively.

In various embodiments, the antibody or antigen binding fragment thereof comprises a heavy chain variable region comprising CDR1, CDR2, and CDR3 regions comprising the amino acid sequences of SEQ ID NOs: 1877, 1878, and 1879, respectively, and/or a light chain variable region comprising CDR1, CDR2, and CDR3 regions comprising the amino acid sequences of SEQ ID NOs: 1880, 1881, and 1882, respectively.

In various embodiments, the antibody or antigen binding fragment thereof comprises a heavy chain variable region comprising CDR1, CDR2, and CDR3 regions comprising the amino acid sequences of SEQ ID NOs: 1883, 1884, and 1885, respectively, and/or a light chain variable region comprising CDR1, CDR2, and CDR3 regions comprising the amino acid sequences of SEQ ID NOs: 1886, 1887, and 1888, respectively.

In various embodiments, the antibody or antigen binding fragment thereof comprises a heavy chain variable region comprising CDR1, CDR2, and CDR3 regions comprising the amino acid sequences of SEQ ID NOs: 1889, 1890, and 1891, respectively, and/or a light chain variable region comprising CDR1, CDR2, and CDR3 regions comprising the amino acid sequences of SEQ ID NOs: 1892, 1893, and 1894, respectively.

In various embodiments, the antibody or antigen binding fragment thereof comprises a heavy chain variable region comprising CDR1, CDR2, and CDR3 regions comprising the amino acid sequences of SEQ ID NOs: 1895, 1896, and 1897, respectively, and/or a light chain variable region comprising CDR1, CDR2, and CDR3 regions comprising the amino acid sequences of SEQ ID NOs: 1898, 1899, and 1900, respectively.

In various embodiments, the antibody or antigen binding fragment thereof comprises a heavy chain variable region comprising CDR1, CDR2, and CDR3 regions comprising the amino acid sequences of SEQ ID NOs: 1901, 1902, and 1903, respectively, and/or a light chain variable region comprising CDR1, CDR2, and CDR3 regions comprising the amino acid sequences of SEQ ID NOs: 1904, 1905, and 1906, respectively.

In various embodiments, the antibody or antigen binding fragment thereof comprises a heavy chain variable region comprising CDR1, CDR2, and CDR3 regions comprising the amino acid sequences of SEQ ID NOs: 1907, 1908, and 1909, respectively, and/or a light chain variable region comprising CDR1, CDR2, and CDR3 regions comprising the amino acid sequences of SEQ ID NOs: 1910, 1911, and 1912, respectively.

In various embodiments, the antibody or antigen binding fragment thereof comprises a heavy chain variable region comprising CDR1, CDR2, and CDR3 regions comprising the amino acid sequences of SEQ ID NOs: 1913, 1914, and 1915, respectively, and/or a light chain variable region comprising CDR1, CDR2, and CDR3 regions comprising the amino acid sequences of SEQ ID NOs: 1916, 1917, and 1918, respectively.

In various embodiments, the antibody or antigen binding fragment thereof comprises a heavy chain variable region comprising CDR1, CDR2, and CDR3 regions comprising the amino acid sequences of SEQ ID NOs: 1919, 1920, and 1921, respectively, and/or a light chain variable region comprising CDR1, CDR2, and CDR3 regions comprising the amino acid sequences of SEQ ID NOs: 1922, 1923, and 1924, respectively.

In various embodiments, the antibody or antigen binding fragment thereof comprises a heavy chain variable region comprising CDR1, CDR2, and CDR3 regions comprising the amino acid sequences of SEQ ID NOs: 1925, 1926, and 1927, respectively, and/or a light chain variable region comprising CDR1, CDR2, and CDR3 regions comprising the amino acid sequences of SEQ ID NOs: 1928, 1929, and 1930, respectively.

In various embodiments, the antibody or antigen binding fragment thereof comprises a heavy chain variable region comprising CDR1, CDR2, and CDR3 regions comprising the amino acid sequences of SEQ ID NOs: 1931, 1932, and 1933, respectively, and/or a light chain variable region comprising CDR1, CDR2, and CDR3 regions comprising the amino acid sequences of SEQ ID NOs: 1934, 1935, and 1936, respectively.

In various embodiments, the antibody or antigen binding fragment thereof comprises a heavy chain variable region comprising CDR1, CDR2, and CDR3 regions comprising the amino acid sequences of SEQ ID NOs: 1937, 1938, and 1939, respectively, and/or a light chain variable region comprising CDR1, CDR2, and CDR3 regions comprising the amino acid sequences of SEQ ID NOs: 1940, 1941, and 1942, respectively.

In various embodiments, the antibody or antigen binding fragment thereof comprises a heavy chain variable region comprising CDR1, CDR2, and CDR3 regions comprising the amino acid sequences of SEQ ID NOs: 1943, 1944, and 1945, respectively, and/or a light chain variable region comprising CDR1, CDR2, and CDR3 regions comprising the amino acid sequences of SEQ ID NOs: 1946, 1947, and 1948, respectively.

In various embodiments, the antibody or antigen binding fragment thereof comprises a heavy chain variable region comprising CDR1, CDR2, and CDR3 regions comprising the amino acid sequences of SEQ ID NOs: 1949, 1950, and 1951, respectively, and/or a light chain variable region comprising CDR1, CDR2, and CDR3 regions comprising the amino acid sequences of SEQ ID NOs: 1952, 1953, and 1954, respectively.

In various embodiments, the antibody or antigen binding fragment thereof comprises a heavy chain variable region comprising CDR1, CDR2, and CDR3 regions comprising the amino acid sequences of SEQ ID NOs: 1955, 1956, and 1957, respectively, and/or a light chain variable region comprising CDR1, CDR2, and CDR3 regions comprising the amino acid sequences of SEQ ID NOs: 1958, 1959, and 1960, respectively.

In various embodiments, the antibody or antigen binding fragment thereof comprises a heavy chain variable region comprising CDR1, CDR2, and CDR3 regions comprising the amino acid sequences of SEQ ID NOs: 1961, 1962, and 1963, respectively, and/or a light chain variable region comprising CDR1, CDR2, and CDR3 regions comprising the amino acid sequences of SEQ ID NOs: 1964, 1965, and 1966, respectively.

In various embodiments, the antibody or antigen binding fragment thereof comprises a heavy chain variable region comprising CDR1, CDR2, and CDR3 regions comprising the amino acid sequences of SEQ ID NOs: 1967, 1968, and 1969, respectively, and/or a light chain variable region comprising CDR1, CDR2, and CDR3 regions comprising the amino acid sequences of SEQ ID NOs: 1970, 1971, and 1972, respectively.

In various embodiments, the antibody or antigen binding fragment thereof comprises a heavy chain variable region comprising CDR1, CDR2, and CDR3 regions comprising the amino acid sequences of SEQ ID NOs: 1973, 1974, and 1975, respectively, and/or a light chain variable region comprising CDR1, CDR2, and CDR3 regions comprising the amino acid sequences of SEQ ID NOs: 1976, 1977, and 1978, respectively.

In various embodiments, the antibody or antigen binding fragment thereof comprises a heavy chain variable region comprising CDR1, CDR2, and CDR3 regions comprising the amino acid sequences of SEQ ID NOs: 1979, 1980, and 1981, respectively, and/or a light chain variable region comprising CDR1, CDR2, and CDR3 regions comprising the amino acid sequences of SEQ ID NOs: 1982, 1983, and 1984, respectively.

In various embodiments, the antibody or antigen binding fragment thereof comprises a heavy chain variable region comprising CDR1, CDR2, and CDR3 regions comprising the amino acid sequences of SEQ ID NOs: 1985, 1986, and 1987, respectively, and/or a light chain variable region comprising CDR1, CDR2, and CDR3 regions comprising the amino acid sequences of SEQ ID NOs: 1988, 1989, and 1990, respectively.

In various embodiments, the antibody or antigen binding fragment thereof comprises a heavy chain variable region comprising CDR1, CDR2, and CDR3 regions comprising the amino acid sequences of SEQ ID NOs: 1991, 1992, and 1993, respectively, and/or a light chain variable region comprising CDR1, CDR2, and CDR3 regions comprising the amino acid sequences of SEQ ID NOs: 1994, 1995, and 1996, respectively.

In various embodiments, the antibody or antigen binding fragment thereof comprises a heavy chain variable region comprising CDR1, CDR2, and CDR3 regions comprising the amino acid sequences of SEQ ID NOs: 1997, 1998, and 1999, respectively, and/or a light chain variable region comprising CDR1, CDR2, and CDR3 regions comprising the amino acid sequences of SEQ ID NOs: 2000, 2001, and 2002, respectively.

In various embodiments, the antibody or antigen binding fragment thereof comprises a heavy chain variable region comprising CDR1, CDR2, and CDR3 regions comprising the amino acid sequences of SEQ ID NOs: 2003, 2004, and 2005, respectively, and/or a light chain variable region comprising CDR1, CDR2, and CDR3 regions comprising the amino acid sequences of SEQ ID NOs: 2006, 2007, and 2008, respectively.

In various embodiments, the antibody or antigen binding fragment thereof comprises a heavy chain variable region comprising CDR1, CDR2, and CDR3 regions comprising the amino acid sequences of SEQ ID NOs: 2009, 2010, and 2011, respectively, and/or a light chain variable region comprising CDR1, CDR2, and CDR3 regions comprising the amino acid sequences of SEQ ID NOs: 2012, 2013, and 2014, respectively.

In various embodiments, the antibody or antigen binding fragment thereof comprises a heavy chain variable region comprising CDR1, CDR2, and CDR3 regions comprising the amino acid sequences of SEQ ID NOs: 2015, 2016, and 2017, respectively, and/or a light chain variable region comprising CDR1, CDR2, and CDR3 regions comprising the amino acid sequences of SEQ ID NOs: 2018, 2019, and 2020, respectively.

In various embodiments, the antibody or antigen binding fragment thereof comprises a heavy chain variable region comprising CDR1, CDR2, and CDR3 regions comprising the amino acid sequences of SEQ ID NOs: 2021, 2022, and 2023, respectively, and/or a light chain variable region comprising CDR1, CDR2, and CDR3 regions comprising the amino acid sequences of SEQ ID NOs: 2024, 2025, and 2026, respectively.

In various embodiments, the antibody or antigen binding fragment thereof comprises a heavy chain variable region comprising CDR1, CDR2, and CDR3 regions comprising the amino acid sequences of SEQ ID NOs: 2027, 2028, and 2029, respectively, and/or a light chain variable region comprising CDR1, CDR2, and CDR3 regions comprising the amino acid sequences of SEQ ID NOs: 2030, 2031, and 2032, respectively.

In various embodiments, the antibody or antigen binding fragment thereof comprises a heavy chain variable region comprising CDR1, CDR2, and CDR3 regions comprising the amino acid sequences of SEQ ID NOs: 2033, 2034, and 2035, respectively, and/or a light chain variable region comprising CDR1, CDR2, and CDR3 regions comprising the amino acid sequences of SEQ ID NOs: 2036, 2037, and 2038, respectively.

In various embodiments, the antibody or antigen binding fragment thereof comprises a heavy chain variable region comprising CDR1, CDR2, and CDR3 regions comprising the amino acid sequences of SEQ ID NOs: 2039, 2040, and 2041, respectively, and/or a light chain variable region comprising CDR1, CDR2, and CDR3 regions comprising the amino acid sequences of SEQ ID NOs: 2042, 2043, and 2044, respectively.

In various embodiments, the antibody or antigen binding fragment thereof comprises a heavy chain variable region comprising CDR1, CDR2, and CDR3 regions comprising the amino acid sequences of SEQ ID NOs: 2045, 2046, and 2047, respectively, and/or a light chain variable region comprising CDR1, CDR2, and CDR3 regions comprising the amino acid sequences of SEQ ID NOs: 2048, 2049, and 2050, respectively.

In various embodiments, the antibody or antigen binding fragment thereof comprises a heavy chain variable region comprising CDR1, CDR2, and CDR3 regions comprising the amino acid sequences of SEQ ID NOs: 2051, 2052, and 2053, respectively, and/or a light chain variable region comprising CDR1, CDR2, and CDR3 regions comprising the amino acid sequences of SEQ ID NOs: 2054, 2055, and 2056, respectively.

In various embodiments, the antibody or antigen binding fragment thereof comprises a heavy chain variable region comprising CDR1, CDR2, and CDR3 regions comprising the amino acid sequences of SEQ ID NOs: 2057, 2058, and 2059, respectively, and/or a light chain variable region comprising CDR1, CDR2, and CDR3 regions comprising the amino acid sequences of SEQ ID NOs: 2060, 2061, and 2062, respectively.

In various embodiments, the antibody or antigen binding fragment thereof comprises a heavy chain variable region comprising CDR1, CDR2, and CDR3 regions comprising the amino acid sequences of SEQ ID NOs: 2063, 2064, and 2065, respectively, and/or a light chain variable region comprising CDR1, CDR2, and CDR3 regions comprising the amino acid sequences of SEQ ID NOs: 2066, 2067, and 2068, respectively.

In various embodiments, the antibody or antigen binding fragment thereof comprises a heavy chain variable region comprising CDR1, CDR2, and CDR3 regions comprising the amino acid sequences of SEQ ID NOs: 2069, 2070, and 2071, respectively, and/or a light chain variable region comprising CDR1, CDR2, and CDR3 regions comprising the amino acid sequences of SEQ ID NOs: 2072, 2073, and 2074, respectively.

In various embodiments, the antibody or antigen binding fragment thereof comprises a heavy chain variable region comprising CDR1, CDR2, and CDR3 regions comprising the amino acid sequences of SEQ ID NOs: 2075, 2076, and 2077, respectively, and/or a light chain variable region comprising CDR1, CDR2, and CDR3 regions comprising the amino acid sequences of SEQ ID NOs: 2078, 2079, and 2080, respectively.

In various embodiments, the antibody or antigen binding fragment thereof comprises a heavy chain variable region comprising CDR1, CDR2, and CDR3 regions comprising the amino acid sequences of SEQ ID NOs: 2081, 2082, and 2083, respectively, and/or a light chain variable region comprising CDR1, CDR2, and CDR3 regions comprising the amino acid sequences of SEQ ID NOs: 2084, 2085, and 2086, respectively.

In various embodiments, the antibody or antigen binding fragment thereof comprises a heavy chain variable region comprising CDR1, CDR2, and CDR3 regions comprising the amino acid sequences of SEQ ID NOs: 2087, 2088, and 2089, respectively, and/or a light chain variable region comprising CDR1, CDR2, and CDR3 regions comprising the amino acid sequences of SEQ ID NOs: 2090, 2091, and 2092, respectively.

In various embodiments, the antibody or antigen binding fragment thereof comprises a heavy chain variable region comprising CDR1, CDR2, and CDR3 regions comprising the amino acid sequences of SEQ ID NOs: 2093, 2094, and 2095, respectively, and/or a light chain variable region comprising CDR1, CDR2, and CDR3 regions comprising the amino acid sequences of SEQ ID NOs: 2096, 2097, and 2098, respectively.

In various embodiments, the antibody or antigen binding fragment thereof comprises a heavy chain variable region comprising CDR1, CDR2, and CDR3 regions comprising the amino acid sequences of SEQ ID NOs: 2099, 2100, and 2101, respectively, and/or a light chain variable region comprising CDR1, CDR2, and CDR3 regions comprising the amino acid sequences of SEQ ID NOs: 2102, 2103, and 2104, respectively.

In various embodiments, the antibody or antigen binding fragment thereof comprises a heavy chain variable region comprising CDR1, CDR2, and CDR3 regions comprising the amino acid sequences of SEQ ID NOs: 2105, 2106, and 2107, respectively, and/or a light chain variable region comprising CDR1, CDR2, and CDR3 regions comprising the amino acid sequences of SEQ ID NOs: 2108, 2109, and 2110, respectively.

In various embodiments, the antibody or antigen binding fragment thereof comprises a heavy chain variable region comprising CDR1, CDR2, and CDR3 regions comprising the amino acid sequences of SEQ ID NOs: 2111, 2112, and 2113, respectively, and/or a light chain variable region comprising CDR1, CDR2, and CDR3 regions comprising the amino acid sequences of SEQ ID NOs: 2114, 2115, and 2116, respectively.

In various embodiments, the antibody or antigen binding fragment thereof comprises a heavy chain variable region comprising CDR1, CDR2, and CDR3 regions comprising the amino acid sequences of SEQ ID NOs: 2117, 2118, and 2119, respectively, and/or a light chain variable region comprising CDR1, CDR2, and CDR3 regions comprising the amino acid sequences of SEQ ID NOs: 2120, 2121, and 2122, respectively.

In various embodiments, the antibody or antigen binding fragment thereof comprises a heavy chain variable region comprising CDR1, CDR2, and CDR3 regions comprising the amino acid sequences of SEQ ID NOs: 2123, 2124, and 2125, respectively, and/or a light chain variable region comprising CDR1, CDR2, and CDR3 regions comprising the amino acid sequences of SEQ ID NOs: 2126, 2127, and 2128, respectively.

In various embodiments, the antibody or antigen binding fragment thereof comprises a heavy chain variable region comprising CDR1, CDR2, and CDR3 regions comprising the amino acid sequences of SEQ ID NOs: 2129, 2130, and 2131, respectively, and/or a light chain variable region comprising CDR1, CDR2, and CDR3 regions comprising the amino acid sequences of SEQ ID NOs: 2132, 2133, and 2134, respectively.

In various embodiments, the antibody or antigen binding fragment thereof comprises a heavy chain variable region comprising CDR1, CDR2, and CDR3 regions comprising the amino acid sequences of SEQ ID NOs: 2135, 2136, and 2137, respectively, and/or a light chain variable region comprising CDR1, CDR2, and CDR3 regions comprising the amino acid sequences of SEQ ID NOs: 2138, 2139, and 2140, respectively.

In various embodiments, the antibody or antigen binding fragment thereof comprises a heavy chain variable region comprising CDR1, CDR2, and CDR3 regions comprising the amino acid sequences of SEQ ID NOs: 2141, 2142, and 2143, respectively, and/or a light chain variable region comprising CDR1, CDR2, and CDR3 regions comprising the amino acid sequences of SEQ ID NOs: 2144, 2145, and 2146, respectively.

In various embodiments, the antibody or antigen binding fragment thereof comprises a heavy chain variable region comprising CDR1, CDR2, and CDR3 regions comprising the amino acid sequences of SEQ ID NOs: 2147, 2148, and 2149, respectively, and/or a light chain variable region comprising CDR1, CDR2, and CDR3 regions comprising the amino acid sequences of SEQ ID NOs: 2150, 2151, and 2152, respectively.

In various embodiments, the antibody or antigen binding fragment thereof comprises a heavy chain variable region comprising CDR1, CDR2, and CDR3 regions comprising the amino acid sequences of SEQ ID NOs: 2153, 2154, and 2155, respectively, and/or a light chain variable region comprising CDR1, CDR2, and CDR3 regions comprising the amino acid sequences of SEQ ID NOs: 2156, 2157, and 2158, respectively.

In various embodiments, the antibody or antigen binding fragment thereof comprises a heavy chain variable region comprising CDR1, CDR2, and CDR3 regions comprising the amino acid sequences of SEQ ID NOs: 2159, 2160, and 2161, respectively, and/or a light chain variable region comprising CDR1, CDR2, and CDR3 regions comprising the amino acid sequences of SEQ ID NOs: 2162, 2163, and 2164, respectively.

In various embodiments, the antibody or antigen binding fragment thereof comprises a heavy chain variable region comprising CDR1, CDR2, and CDR3 regions comprising the amino acid sequences of SEQ ID NOs: 2165, 2166, and 2167, respectively, and/or a light chain variable region comprising CDR1, CDR2, and CDR3 regions comprising the amino acid sequences of SEQ ID NOs: 2168, 2169, and 2170, respectively.

In various embodiments, the antibody or antigen binding fragment thereof comprises a heavy chain variable region comprising CDR1, CDR2, and CDR3 regions comprising the amino acid sequences of SEQ ID NOs: 2171, 2172, and 2173, respectively, and/or a light chain variable region comprising CDR1, CDR2, and CDR3 regions comprising the amino acid sequences of SEQ ID NOs: 2174, 2175, and 2176, respectively.

In various embodiments, the antibody or antigen binding fragment thereof comprises a heavy chain variable region comprising CDR1, CDR2, and CDR3 regions comprising the amino acid sequences of SEQ ID NOs: 2177, 2178, and 2179, respectively, and/or a light chain variable region comprising CDR1, CDR2, and CDR3 regions comprising the amino acid sequences of SEQ ID NOs: 2180, 2181, and 2182, respectively.

In various embodiments, the antibody or antigen binding fragment thereof comprises a heavy chain variable region comprising CDR1, CDR2, and CDR3 regions comprising the amino acid sequences of SEQ ID NOs: 2183, 2184, and 2185, respectively, and/or a light chain variable region comprising CDR1, CDR2, and CDR3 regions comprising the amino acid sequences of SEQ ID NOs: 2186, 2187, and 2188, respectively.

In various embodiments, the antibody or antigen binding fragment thereof comprises a heavy chain variable region comprising CDR1, CDR2, and CDR3 regions comprising the amino acid sequences of SEQ ID NOs: 2189, 2190, and 2191, respectively, and/or a light chain variable region comprising CDR1, CDR2, and CDR3 regions comprising the amino acid sequences of SEQ ID NOs: 2192, 2193, and 2194, respectively.

In various embodiments, the antibody or antigen binding fragment thereof comprises a heavy chain variable region comprising CDR1, CDR2, and CDR3 regions comprising the amino acid sequences of SEQ ID NOs: 2195, 2196, and 2197, respectively, and/or a light chain variable region comprising CDR1, CDR2, and CDR3 regions comprising the amino acid sequences of SEQ ID NOs: 2198, 2199, and 2200, respectively.

In various embodiments, the antibody or antigen binding fragment thereof comprises a heavy chain variable region comprising CDR1, CDR2, and CDR3 regions comprising the amino acid sequences of SEQ ID NOs: 2201, 2202, and 2203, respectively, and/or a light chain variable region comprising CDR1, CDR2, and CDR3 regions comprising the amino acid sequences of SEQ ID NOs: 2204, 2205, and 2206, respectively.

In various embodiments, the antibody or antigen binding fragment thereof comprises a heavy chain variable region comprising CDR1, CDR2, and CDR3 regions comprising the amino acid sequences of SEQ ID NOs: 2207, 2208, and 2209, respectively, and/or a light chain variable region comprising CDR1, CDR2, and CDR3 regions comprising the amino acid sequences of SEQ ID NOs: 2210, 2211, and 2212, respectively.

Another aspect of the invention provides an isolated antibody or antigen binding fragment thereof which specifically binds to LAP comprising a heavy chain variable region comprising CDR1, CDR2, and CDR3 regions comprising CDR1, CDR2, and CDR3 amino acid sequences selected from the group of sequences set forth in Table 42, Table 43 and/or Table 45A and a light chain variable region comprising CDR1, CDR2, and CDR3 regions comprising CDR1, CDR2, and CDR3 amino acid sequences selected from the group of sequences set forth in Table 42, Table 43 and/or Table 45A.

In various embodiments, the CDR1, CDR2, and CDR3 amino acid sequences in the heavy chain variable region and/or the CDR1, CDR2, and CDR3 amino acid sequences in the light chain variable region are selected from the group of sequences set forth in SEQ ID NOs: 1565-2212. In various embodiments, the CDR1, CDR2, and CDR3 amino acid sequences in the heavy chain variable region and/or the light chain variable region comprising CDR1, CDR2, and CDR3 amino acid sequences are selected from the group of sequences set forth in SEQ ID NOs: 2229-2570. In various embodiments, the CDR1, CDR2, and CDR3 amino acid sequences in the heavy chain variable region and the CDR1, CDR2, and CDR3 amino acid sequences in the light chain variable region are selected from the group of sequences set forth in SEQ ID NOs: 2229-2570.

In various embodiments, the CDR1, CDR2, and CDR3 regions in the heavy chain variable region and the light chain variable region comprising CDR1, CDR2, and CDR3 amino acid sequences selected from the group of sequences set forth in SEQ ID NOs: 1565-2212 as described in Table 43. In various embodiments, the CDR1, CDR2, and CDR3 regions in the heavy chain variable region and the light chain variable region comprising CDR1, CDR2, and CDR3 amino acid sequences selected from the group of sequences set forth in SEQ ID NOs: 2229-2570 as set forth in Table 45A. In various embodiments, the CDR1, CDR2, and CDR3 regions in the heavy chain variable region and the light chain variable region comprising CDR1, CDR2, and CDR3 amino acid sequences as described below:

Kabat

| heavy chain variable region | | | light chain variable region | | |
|---|---|---|---|---|---|
| CDR1 | CDR2 | CDR3 | CDR1 | CDR2 | CDR3 |
| 2229 | 2230 | 2231 | 2232 | 2233 | 2234 |
| 2253 | 2254 | 2255 | 2256 | 2257 | 2258 |
| 2277 | 2278 | 2279 | 2280 | 2281 | 2282 |
| 2301 | 2302 | 2303 | 2304 | 2305 | 2306 |
| 2325 | 2326 | 2327 | 2328 | 2329 | 2330 |
| 2349 | 2350 | 2351 | 2352 | 2353 | 2354 |
| 2373 | 2374 | 2375 | 2376 | 2377 | 2378 |
| 2397 | 2398 | 2399 | 2400 | 2401 | 2402 |
| 2421 | 2422 | 2423 | 2424 | 2425 | 2426 |
| 2445 | 2446 | 2447 | 2448 | 2449 | 2450 |
| 2469 | 2470 | 2471 | 2472 | 2473 | 2474 |
| 2493 | 2494 | 2495 | 2496 | 2497 | 2498 |
| 2517 | 2518 | 2519 | 2520 | 2521 | 2522 |
| 2541 | 2542 | 2543 | 2544 | 2545 | 2546 |
| 2565 | 2566 | 2567 | 2568 | 2569 | 2570 |

In various embodiments, the CDR1, CDR2, and CDR3 regions amino acid sequences in the heavy chain variable region and the light chain variable region comprising CDR1, CDR2, and CDR3 amino acid sequences as described in the light chain variable region are selected from the below table:

Chothia

| heavy chain variable region | | | light chain variable region | | |
|---|---|---|---|---|---|
| CDR1 | CDR2 | CDR3 | CDR1 | CDR2 | CDR3 |
| 2235 | 2236 | 2237 | 2238 | 2239 | 2240 |
| 2259 | 2260 | 2261 | 2262 | 2263 | 2264 |
| 2283 | 2284 | 2285 | 2286 | 2287 | 2288 |
| 2307 | 2308 | 2309 | 2310 | 2311 | 2312 |
| 2331 | 2332 | 2333 | 2334 | 2335 | 2336 |
| 2355 | 2356 | 2357 | 2358 | 2359 | 2360 |
| 2379 | 2380 | 2381 | 2382 | 2383 | 2384 |
| 2403 | 2404 | 2405 | 2406 | 2407 | 2408 |
| 2427 | 2428 | 2429 | 2430 | 2431 | 2432 |
| 2451 | 2452 | 2453 | 2454 | 2455 | 2456 |
| 2475 | 2476 | 2477 | 2478 | 2479 | 2480 |
| 2499 | 2500 | 2501 | 2502 | 2503 | 2504 |
| 2523 | 2524 | 2525 | 2526 | 2527 | 2528 |
| 2547 | 2548 | 2549 | 2550 | 2551 | 2552 |
| 2571 | 2572 | 2573 | 2574 | 2575 | 2576 |

In various embodiments, the CDR1, CDR2, and CDR3 regions amino acid sequences in the heavy chain variable region and the light chain variable region comprising CDR1, CDR2, and CDR3 amino acid sequences as described in the light chain variable region are selected from the below table:

ABM

| heavy chain variable region | | | light chain variable region | | |
|---|---|---|---|---|---|
| CDR1 | CDR2 | CDR3 | CDR1 | CDR2 | CDR3 |
| 2241 | 2242 | 2243 | 2244 | 2245 | 2246 |
| 2265 | 2266 | 2267 | 2268 | 2269 | 2270 |
| 2289 | 2290 | 2291 | 2292 | 2293 | 2294 |
| 2313 | 2314 | 2315 | 2316 | 2317 | 2318 |
| 2337 | 2338 | 2339 | 2340 | 2341 | 2342 |
| 2361 | 2362 | 2363 | 2364 | 2365 | 2366 |
| 2385 | 2386 | 2387 | 2388 | 2389 | 2390 |
| 2409 | 2410 | 2411 | 2412 | 2413 | 2414 |
| 2433 | 2434 | 2435 | 2436 | 2437 | 2438 |
| 2457 | 2458 | 2459 | 2460 | 2461 | 2462 |
| 2481 | 2482 | 2483 | 2484 | 2485 | 2486 |
| 2505 | 2506 | 2507 | 2508 | 2509 | 2510 |
| 2529 | 2530 | 2531 | 2532 | 2533 | 2534 |
| 2553 | 2554 | 2555 | 2556 | 2557 | 2558 |
| 2577 | 2578 | 2579 | 2580 | 2581 | 2582 |

In various embodiments, the CDR1, CDR2, and CDR3 amino acid sequences in the heavy chain variable region and the CDR1, CDR2, and CDR3 amino acid sequences in the light chain variable region are selected from the below table:

IMGT

| heavy chain variable region | | | light chain variable region | | |
|---|---|---|---|---|---|
| CDR1 | CDR2 | CDR3 | CDR1 | CDR2 | CDR3 |
| 2247 | 2248 | 2249 | 2250 | 2251 | 2252 |
| 2271 | 2272 | 2273 | 2274 | 2275 | 2276 |
| 2295 | 2296 | 2297 | 2298 | 2299 | 2300 |
| 2319 | 2320 | 2321 | 2322 | 2323 | 2324 |
| 2343 | 2344 | 2345 | 2346 | 2347 | 2348 |
| 2367 | 2368 | 2369 | 2370 | 2371 | 2372 |
| 2391 | 2392 | 2393 | 2394 | 2395 | 2396 |
| 2415 | 2416 | 2417 | 2418 | 2419 | 2420 |
| 2439 | 2440 | 2441 | 2442 | 2443 | 2444 |
| 2463 | 2464 | 2465 | 2466 | 2467 | 2468 |
| 2487 | 2488 | 2489 | 2490 | 2491 | 2492 |
| 2511 | 2512 | 2513 | 2514 | 2515 | 2516 |
| 2535 | 2536 | 2537 | 2538 | 2539 | 2540 |
| 2559 | 2560 | 2561 | 2562 | 2563 | 2564 |
| 2583 | 2584 | 2585 | 2586 | 2587 | 2588 |

In various embodiments, the antibody or antigen binding fragment thereof comprises a heavy chain variable region sequence comprising an amino acid sequence selected from the group consisting of SEQ ID NOs: 575-622, 661-685, 712-756, 794-827, 849-893; 921-950, 971-1009, 1037-1067, 1089-1113, 1138-1179, and 2589-2603.

In various embodiments, the antibody or antigen binding fragment thereof comprises a heavy chain variable region sequence which is at least 85%, 90%, 95%, 98%, or 99% identical to an amino acid sequence selected from the group consisting of SEQ ID NOs: 575-622, 661-685, 712-756, 794-827, 849-893; 921-950, 971-1009, 1037-1067, 1089-1113, 1138-1179, and −2589-2603.

In various embodiments, the antibody or antigen binding fragment thereof comprises a light chain variable region sequence selected from the group consisting of SEQ ID NOs: 623-660; 686-711, 757-793; 828-848, 894-920, 951-970, 1010-1036, 1068-1088, 1114-1137, and 1180-1211.

In various embodiments, the antibody or antigen binding fragment thereof comprises a light chain variable region sequence which is at least 85%, 90%, 95%, 98%, or 99% identical to the amino acid sequence selected from the group consisting of SEQ ID NOs: 623-660; 686-711, 757-793; 828-848, 894-920, 951-970, 1010-1036, 1068-1088, 1114-1137, and 1180-1211.

In various embodiments, the antibody or antigen binding fragment thereof comprises a heavy chain variable region sequence comprising the amino acid sequence selected from the group consisting of SEQ ID NOs: 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 458, 468, 478, 488, 498, 508, 518, 528, and 538.

In various embodiments, the antibody or antigen binding fragment thereof comprises a heavy chain variable region sequence which is at least 85%, 90%, 95%, 98%, or 99% identical to the amino acid sequence selected from the group consisting of SEQ ID NOs: 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 458, 468, 478, 488, 498, 508, 518, 528, and 538.

In various embodiments, the antibody or antigen binding fragment thereof comprises a light chain variable region sequence comprising the amino acid sequence selected from the group consisting of SEQ ID NOs: 103, 113, 123, 133, 143, 153, 163, 173, 183, 193, 203, 213, 223, 233, 243, 253, 263, 273, 283, 293, 303, 313, 323, 333, 343, 353, 363, 373, 383, 393, 403, 413, 423, 433, 443, 463, 473, 483, 493, 503, 513, 523, 533, and 543.

In various embodiments, the antibody or antigen binding fragment thereof comprises a light chain variable region sequence which is at least 85%, 90%, 95%, 98%, or 99% identical to the amino acid sequence selected from the group consisting of SEQ ID NOs: 103, 113, 123, 133, 143, 153, 163, 173, 183, 193, 203, 213, 223, 233, 243, 253, 263, 273, 283, 293, 303, 313, 323, 333, 343, 353, 363, 373, 383, 393, 403, 413, 423, 433, 443, 463, 473, 483, 493, 503, 513, 523, 533, and 543.

In various embodiments, the antibody or antigen binding fragment thereof comprises a heavy chain variable region sequence comprising an amino acid sequence selected from the group consisting of SEQ ID NOs: 548, 558, and 568; or comprises a heavy chain variable region sequence which is at least 85%, 90%, 95%, 98%, or 99% identical to an amino acid sequence selected from the group consisting of SEQ ID NOs: 548, 558, and 568.

In various embodiments, the antibody or antigen binding fragment thereof comprises a light chain variable region sequence comprising an amino acid sequence selected from the group consisting of SEQ ID NOs: 553, 563, and 573; or comprises a light chain variable region sequence which is at least 85%, 90%, 95%, 98%, or 99% identical to an amino acid sequence selected from the group consisting of SEQ ID NOs: 548, 558, and 568.

In various embodiments, the antibody or antigen binding fragment thereof comprises a heavy chain variable region sequence comprising an amino acid sequence selected from the group consisting of SEQ ID NOs: 46, 51, 56, 61, 66, 71, 76, 81, 86, and 91; or comprises a heavy chain variable region sequence which is at least 85%, 90%, 95%, 98%, or 99% identical to an amino acid sequence selected from the group consisting of SEQ ID NOs: 46, 51, 56, 61, 66, 71, 76, 81, 86, and 91.

In various embodiments, the antibody or antigen binding fragment thereof comprises a light chain variable region sequence comprising an amino acid sequence selected from the group consisting of SEQ ID NOs: 11, 16, 21, 26, 31, 36, and 41; or comprises a light chain variable region sequence which is at least 85%, 90%, 95%, 98%, or 99% identical to an amino acid sequence selected from the group consisting of SEQ ID NOs: 11, 16, 21, 26, 31, 36, and 41.

In various embodiments, the antibody or antigen binding fragment thereof comprises a heavy chain variable region sequence comprising the amino acid sequence of SEQ ID NO: 1212; or comprises a heavy chain variable region sequence which is at least 85%, 90%, 95%, 98%, or 99% identical to the amino acid sequence of SEQ ID NO: 1212.

In various embodiments, the antibody or antigen binding fragment thereof comprises a light chain variable region sequence comprising the amino acid sequence of SEQ ID NO: 1216; or comprises a light chain variable region sequence which is at least 85%, 90%, 95%, 98%, or 99% identical to the amino acid sequence of SEQ ID NO: 1216.

In various embodiments, the antibody or antigen binding fragment thereof comprises a heavy chain variable region sequence comprising the amino acid sequence of SEQ ID NO: 1220; or comprises a heavy chain variable region sequence which is at least 85%, 90%, 95%, 98%, or 99% identical to the amino acid sequence of SEQ ID NO: 1220.

In various embodiments, the antibody or antigen binding fragment thereof comprises a light chain variable region sequence comprising the amino acid sequence of SEQ ID NO: 1224; or comprises a light chain variable region sequence which is at least 85%, 90%, 95%, 98%, or 99% identical to the amino acid sequence of SEQ ID NO: 1224.

In various embodiments, the antibody or antigen binding fragment thereof comprises a heavy chain variable region sequence comprising the amino acid sequence of SEQ ID NO: 1228; or comprises a heavy chain variable region sequence which is at least 85%, 90%, 95%, 98%, or 99% identical to the amino acid sequence of SEQ ID NO: 1228.

In various embodiments, the antibody or antigen binding fragment thereof comprises a light chain variable region sequence comprising the amino acid sequence of SEQ ID NO: 1232; or comprises a light chain variable region sequence which is at least 85%, 90%, 95%, 98%, or 99% identical to the amino acid sequence of SEQ ID NO: 1232.

In various embodiments, the antibody or antigen binding fragment thereof comprises a heavy chain variable region sequence comprising the amino acid sequence of SEQ ID NO: 1236; or comprises a heavy chain variable region sequence which is at least 85%, 90%, 95%, 98%, or 99% identical to the amino acid sequence of SEQ ID NO: 1236.

In various embodiments, the antibody or antigen binding fragment thereof comprises a light chain variable region sequence comprising the amino acid sequence of SEQ ID NO: 1240; or comprises a light chain variable region sequence which is at least 85%, 90%, 95%, 98%, or 99% identical to the amino acid sequence of SEQ ID NO: 1240.

In various embodiments, the antibody or antigen binding fragment thereof comprises a heavy chain variable region sequence comprising the amino acid sequence of SEQ ID NO: 1244; or comprises a heavy chain variable region sequence which is at least 85%, 90%, 95%, 98%, or 99% identical to the amino acid sequence of SEQ ID NO: 1244.

In various embodiments, the antibody or antigen binding fragment thereof comprises a light chain variable region sequence comprising the amino acid sequence of SEQ ID NO: 1248; or comprises a light chain variable region sequence which is at least 85%, 90%, 95%, 98%, or 99% identical to the amino acid sequence of SEQ ID NO: 1248.

In various embodiments, the antibody or antigen binding fragment thereof comprises a heavy chain variable region sequence comprising the amino acid sequence of SEQ ID NO: 1252; or comprises a heavy chain variable region sequence which is at least 85%, 90%, 95%, 98%, or 99% identical to the amino acid sequence of SEQ ID NO: 1252.

In various embodiments, the antibody or antigen binding fragment thereof comprises a light chain variable region sequence comprising the amino acid sequence of SEQ ID NO: 1256; or comprises a light chain variable region sequence which is at least 85%, 90%, 95%, 98%, or 99% identical to the amino acid sequence of SEQ ID NO: 1256.

In various embodiments, the antibody or antigen binding fragment thereof comprises a heavy chain variable region sequence comprising the amino acid sequence of SEQ ID NO: 1260; or comprises a heavy chain variable region sequence which is at least 85%, 90%, 95%, 98%, or 99% identical to the amino acid sequence of SEQ ID NO: 1260.

In various embodiments, the antibody or antigen binding fragment thereof comprises a light chain variable region sequence comprising the amino acid sequence of SEQ ID NO: 1264; or comprises a light chain variable region sequence which is at least 85%, 90%, 95%, 98%, or 99% identical to the amino acid sequence of SEQ ID NO: 1264.

In various embodiments, the antibody or antigen binding fragment thereof comprises a heavy chain variable region sequence comprising the amino acid sequence of SEQ ID NO: 1268; or comprises a heavy chain variable region sequence which is at least 85%, 90%, 95%, 98%, or 99% identical to the amino acid sequence of SEQ ID NO: 1268.

In various embodiments, the antibody or antigen binding fragment thereof comprises a light chain variable region sequence comprising the amino acid sequence of SEQ ID NO: 1272; or comprises a light chain variable region sequence which is at least 85%, 90%, 95%, 98%, or 99% identical to the amino acid sequence of SEQ ID NO: 1272.

In various embodiments, the antibody or antigen binding fragment thereof comprises a heavy chain variable region sequence comprising the amino acid sequence of SEQ ID NO: 1276; or comprises a heavy chain variable region sequence which is at least 85%, 90%, 95%, 98%, or 99% identical to the amino acid sequence of SEQ ID NO: 1276.

In various embodiments, the antibody or antigen binding fragment thereof comprises a light chain variable region sequence comprising the amino acid sequence of SEQ ID NO: 1280; or comprises a light chain variable region sequence which is at least 85%, 90%, 95%, 98%, or 99% identical to the amino acid sequence of SEQ ID NO: 1280.

In various embodiments, the antibody or antigen binding fragment thereof comprises a heavy chain variable region sequence comprising the amino acid sequence of SEQ ID NO: 1284; or comprises a heavy chain variable region sequence which is at least 85%, 90%, 95%, 98%, or 99% identical to the amino acid sequence of SEQ ID NO: 1284.

In various embodiments, the antibody or antigen binding fragment thereof comprises a light chain variable region sequence comprising the amino acid sequence of SEQ ID NO: 1288; or comprises a light chain variable region sequence which is at least 85%, 90%, 95%, 98%, or 99% identical to the amino acid sequence of SEQ ID NO: 1288.

In various embodiments, the antibody or antigen binding fragment thereof comprises a heavy chain variable region sequence comprising the amino acid sequence of SEQ ID NO: 1295; or comprises a heavy chain variable region sequence which is at least 85%, 90%, 95%, 98%, or 99% identical to the amino acid sequence of SEQ ID NO: 1295.

In various embodiments, the antibody or antigen binding fragment thereof comprises a light chain variable region sequence comprising the amino acid sequence of SEQ ID NO: 1300; or comprises a light chain variable region sequence which is at least 85%, 90%, 95%, 98%, or 99% identical to the amino acid sequence of SEQ ID NO: 1300.

In various embodiments, the antibody or antigen binding fragment thereof comprises a heavy chain variable region sequence comprising the amino acid sequence of SEQ ID NO: 1305; or comprises a heavy chain variable region sequence which is at least 85%, 90%, 95%, 98%, or 99% identical to the amino acid sequence of SEQ ID NO: 1305.

In various embodiments, the antibody or antigen binding fragment thereof comprises a light chain variable region sequence comprising the amino acid sequence of SEQ ID NO: 1310; or comprises a light chain variable region sequence which is at least 85%, 90%, 95%, 98%, or 99% identical to the amino acid sequence of SEQ ID NO: 1310.

In various embodiments, the antibody or antigen binding fragment thereof comprises a heavy chain variable region sequence comprising the amino acid sequence of SEQ ID NO: 1315; or comprises a heavy chain variable region sequence which is at least 85%, 90%, 95%, 98%, or 99% identical to the amino acid sequence of SEQ ID NO: 1315.

In various embodiments, the antibody or antigen binding fragment thereof comprises a light chain variable region sequence comprising the amino acid sequence of SEQ ID NO: 1320; or comprises a light chain variable region sequence which is at least 85%, 90%, 95%, 98%, or 99% identical to the amino acid sequence of SEQ ID NO: 1320.

In various embodiments, the antibody or antigen binding fragment thereof comprises a heavy chain variable region sequence comprising the amino acid sequence of SEQ ID NO: 1325; or comprises a heavy chain variable region sequence which is at least 85%, 90%, 95%, 98%, or 99% identical to the amino acid sequence of SEQ ID NO: 1325.

In various embodiments, the antibody or antigen binding fragment thereof comprises a light chain variable region sequence comprising the amino acid sequence of SEQ ID NO: 1330; or comprises a light chain variable region sequence which is at least 85%, 90%, 95%, 98%, or 99% identical to the amino acid sequence of SEQ ID NO: 1330.

In various embodiments, the antibody or antigen binding fragment thereof comprises a heavy chain variable region sequence comprising the amino acid sequence of SEQ ID NO: 1335; or comprises a heavy chain variable region sequence which is at least 85%, 90%, 95%, 98%, or 99% identical to the amino acid sequence of SEQ ID NO: 1335.

In various embodiments, the antibody or antigen binding fragment thereof comprises a light chain variable region sequence comprising the amino acid sequence of SEQ ID NO: 1340; or comprises a light chain variable region sequence which is at least 85%, 90%, 95%, 98%, or 99% identical to the amino acid sequence of SEQ ID NO: 1340.

In various embodiments, the antibody or antigen binding fragment thereof comprises a heavy chain variable region sequence comprising the amino acid sequence of SEQ ID NO: 1345; or comprises a heavy chain variable region sequence which is at least 85%, 90%, 95%, 98%, or 99% identical to the amino acid sequence of SEQ ID NO: 1345.

In various embodiments, the antibody or antigen binding fragment thereof comprises a light chain variable region sequence comprising the amino acid sequence of SEQ ID NO: 1350; or comprises a light chain variable region sequence which is at least 85%, 90%, 95%, 98%, or 99% identical to the amino acid sequence of SEQ ID NO: 1350.

In various embodiments, the antibody or antigen binding fragment thereof comprises a heavy chain variable region sequence comprising the amino acid sequence of SEQ ID NO: 1355; or comprises a heavy chain variable region sequence which is at least 85%, 90%, 95%, 98%, or 99% identical to the amino acid sequence of SEQ ID NO: 1355.

In various embodiments, the antibody or antigen binding fragment thereof comprises a light chain variable region sequence comprising the amino acid sequence of SEQ ID NO: 1360; or comprises a light chain variable region sequence which is at least 85%, 90%, 95%, 98%, or 99% identical to the amino acid sequence of SEQ ID NO: 1360.

In various embodiments, the antibody or antigen binding fragment thereof comprises a heavy chain variable region sequence comprising the amino acid sequence of SEQ ID NO: 1365; or comprises a heavy chain variable region sequence which is at least 85%, 90%, 95%, 98%, or 99% identical to the amino acid sequence of SEQ ID NO: 1365.

In various embodiments, the antibody or antigen binding fragment thereof comprises a light chain variable region sequence comprising the amino acid sequence of SEQ ID NO: 1370; or comprises a light chain variable region sequence which is at least 85%, 90%, 95%, 98%, or 99% identical to the amino acid sequence of SEQ ID NO: 1370.

In various embodiments, the antibody or antigen binding fragment thereof comprises a heavy chain variable region sequence comprising the amino acid sequence of SEQ ID NO: 1375; or comprises a heavy chain variable region sequence which is at least 85%, 90%, 95%, 98%, or 99% identical to the amino acid sequence of SEQ ID NO: 1375.

In various embodiments, the antibody or antigen binding fragment thereof comprises a light chain variable region sequence comprising the amino acid sequence of SEQ ID NO: 1380; or comprises a light chain variable region sequence which is at least 85%, 90%, 95%, 98%, or 99% identical to the amino acid sequence of SEQ ID NO: 1380.

In various embodiments, the antibody or antigen binding fragment thereof comprises a heavy chain variable region sequence comprising the amino acid sequence of SEQ ID NO: 1385; or comprises a heavy chain variable region sequence which is at least 85%, 90%, 95%, 98%, or 99% identical to the amino acid sequence of SEQ ID NO: 1385.

In various embodiments, the antibody or antigen binding fragment thereof comprises a light chain variable region sequence comprising the amino acid sequence of SEQ ID NO: 1390; or comprises a light chain variable region sequence which is at least 85%, 90%, 95%, 98%, or 99% identical to the amino acid sequence of SEQ ID NO: 1390.

In various embodiments, the antibody or antigen binding fragment thereof comprises a heavy chain variable region sequence comprising the amino acid sequence of SEQ ID NO: 1395; or comprises a heavy chain variable region sequence which is at least 85%, 90%, 95%, 98%, or 99% identical to the amino acid sequence of SEQ ID NO: 1395.

In various embodiments, the antibody or antigen binding fragment thereof comprises a light chain variable region sequence comprising the amino acid sequence of SEQ ID NO: 1400; or comprises a light chain variable region sequence which is at least 85%, 90%, 95%, 98%, or 99% identical to the amino acid sequence of SEQ ID NO: 1400.

In various embodiments, the antibody or antigen binding fragment thereof comprises a heavy chain variable region sequence comprising the amino acid sequence of SEQ ID NO: 1405; or comprises a heavy chain variable region sequence which is at least 85%, 90%, 95%, 98%, or 99% identical to the amino acid sequence of SEQ ID NO: 1405.

In various embodiments, the antibody or antigen binding fragment thereof comprises a light chain variable region sequence comprising the amino acid sequence of SEQ ID NO: 1410; or comprises a light chain variable region sequence which is at least 85%, 90%, 95%, 98%, or 99% identical to the amino acid sequence of SEQ ID NO: 1410.

In various embodiments, the antibody or antigen binding fragment thereof comprises a heavy chain variable region sequence comprising the amino acid sequence of SEQ ID NO: 1415; or comprises a heavy chain variable region sequence which is at least 85%, 90%, 95%, 98%, or 99% identical to the amino acid sequence of SEQ ID NO: 1415.

In various embodiments, the antibody or antigen binding fragment thereof comprises a light chain variable region sequence comprising the amino acid sequence of SEQ ID NO: 1420; or comprises a light chain variable region sequence which is at least 85%, 90%, 95%, 98%, or 99% identical to the amino acid sequence of SEQ ID NO: 1420.

In various embodiments, the antibody or antigen binding fragment thereof comprises a heavy chain variable region sequence comprising the amino acid sequence of SEQ ID NO: 1425; or comprises a heavy chain variable region sequence which is at least 85%, 90%, 95%, 98%, or 99% identical to the amino acid sequence of SEQ ID NO: 1425.

In various embodiments, the antibody or antigen binding fragment thereof comprises a light chain variable region sequence comprising the amino acid sequence of SEQ ID NO: 1430; or comprises a light chain variable region sequence which is at least 85%, 90%, 95%, 98%, or 99% identical to the amino acid sequence of SEQ ID NO: 1430.

In various embodiments, the antibody or antigen binding fragment thereof comprises a heavy chain variable region sequence comprising the amino acid sequence of SEQ ID NO: 1435; or comprises a heavy chain variable region sequence which is at least 85%, 90%, 95%, 98%, or 99% identical to the amino acid sequence of SEQ ID NO: 1435.

In various embodiments, the antibody or antigen binding fragment thereof comprises a light chain variable region sequence comprising the amino acid sequence of SEQ ID NO: 1440; or comprises a light chain variable region sequence which is at least 85%, 90%, 95%, 98%, or 99% identical to the amino acid sequence of SEQ ID NO: 1440.

In various embodiments, the antibody or antigen binding fragment thereof comprises a heavy chain variable region sequence comprising the amino acid sequence of SEQ ID NO: 1445; or comprises a heavy chain variable region sequence which is at least 85%, 90%, 95%, 98%, or 99% identical to the amino acid sequence of SEQ ID NO: 1445.

In various embodiments, the antibody or antigen binding fragment thereof comprises a light chain variable region sequence comprising the amino acid sequence of SEQ ID NO: 1450; or comprises a light chain variable region sequence which is at least 85%, 90%, 95%, 98%, or 99% identical to the amino acid sequence of SEQ ID NO: 1450.

In various embodiments, the antibody or antigen binding fragment thereof comprises a heavy chain variable region sequence comprising the amino acid sequence of SEQ ID NO: 1455; or comprises a heavy chain variable region sequence which is at least 85%, 90%, 95%, 98%, or 99% identical to the amino acid sequence of SEQ ID NO: 1455.

In various embodiments, the antibody or antigen binding fragment thereof comprises a light chain variable region sequence comprising the amino acid sequence of SEQ ID NO: 1460; or comprises a light chain variable region sequence which is at least 85%, 90%, 95%, 98%, or 99% identical to the amino acid sequence of SEQ ID NO: 1460.

In various embodiments, the antibody or antigen binding fragment thereof comprises a heavy chain variable region sequence comprising the amino acid sequence of SEQ ID NO: 1465; or comprises a heavy chain variable region sequence which is at least 85%, 90%, 95%, 98%, or 99% identical to the amino acid sequence of SEQ ID NO: 1465.

In various embodiments, the antibody or antigen binding fragment thereof comprises a light chain variable region sequence comprising the amino acid sequence of SEQ ID NO: 1470; or comprises a light chain variable region sequence which is at least 85%, 90%, 95%, 98%, or 99% identical to the amino acid sequence of SEQ ID NO: 1470.

In various embodiments, the antibody or antigen binding fragment thereof comprises a heavy chain variable region sequence comprising the amino acid sequence of SEQ ID NO: 1475; or comprises a heavy chain variable region sequence which is at least 85%, 90%, 95%, 98%, or 99% identical to the amino acid sequence of SEQ ID NO: 1475.

In various embodiments, the antibody or antigen binding fragment thereof comprises a light chain variable region sequence comprising the amino acid sequence of SEQ ID NO: 1480; or comprises a light chain variable region sequence which is at least 85%, 90%, 95%, 98%, or 99% identical to the amino acid sequence of SEQ ID NO: 1480.

In various embodiments, the antibody or antigen binding fragment thereof comprises a heavy chain variable region sequence comprising the amino acid sequence of SEQ ID NO: 1485; or comprises a heavy chain variable region sequence which is at least 85%, 90%, 95%, 98%, or 99% identical to the amino acid sequence of SEQ ID NO: 1485.

In various embodiments, the antibody or antigen binding fragment thereof comprises a light chain variable region sequence comprising the amino acid sequence of SEQ ID NO: 1490; or comprises a light chain variable region sequence which is at least 85%, 90%, 95%, 98%, or 99% identical to the amino acid sequence of SEQ ID NO: 1490.

In various embodiments, the antibody or antigen binding fragment thereof comprises a heavy chain variable region sequence comprising the amino acid sequence of SEQ ID NO: 1495; or comprises a heavy chain variable region sequence which is at least 85%, 90%, 95%, 98%, or 99% identical to the amino acid sequence of SEQ ID NO: 1495.

In various embodiments, the antibody or antigen binding fragment thereof comprises a light chain variable region sequence comprising the amino acid sequence of SEQ ID NO: 1500; or comprises a light chain variable region sequence which is at least 85%, 90%, 95%, 98%, or 99% identical to the amino acid sequence of SEQ ID NO: 1500.

In various embodiments, the antibody or antigen binding fragment thereof comprises a heavy chain variable region sequence comprising the amino acid sequence of SEQ ID NO: 1505; or comprises a heavy chain variable region sequence which is at least 85%, 90%, 95%, 98%, or 99% identical to the amino acid sequence of SEQ ID NO: 1505.

In various embodiments, the antibody or antigen binding fragment thereof comprises a light chain variable region sequence comprising the amino acid sequence of SEQ ID NO: 1510; or comprises a light chain variable region sequence which is at least 85%, 90%, 95%, 98%, or 99% identical to the amino acid sequence of SEQ ID NO: 1510.

In various embodiments, the antibody or antigen binding fragment thereof comprises a heavy chain variable region sequence comprising the amino acid sequence of SEQ ID NO: 1515; or comprises a heavy chain variable region sequence which is at least 85%, 90%, 95%, 98%, or 99% identical to the amino acid sequence of SEQ ID NO: 1515.

In various embodiments, the antibody or antigen binding fragment thereof comprises a light chain variable region sequence comprising the amino acid sequence of SEQ ID NO: 1520; or comprises a light chain variable region sequence which is at least 85%, 90%, 95%, 98%, or 99% identical to the amino acid sequence of SEQ ID NO: 1520.

In various embodiments, the antibody or antigen binding fragment thereof comprises a heavy chain variable region sequence comprising the amino acid sequence of SEQ ID NO: 1525; or comprises a heavy chain variable region sequence which is at least 85%, 90%, 95%, 98%, or 99% identical to the amino acid sequence of SEQ ID NO: 1525.

In various embodiments, the antibody or antigen binding fragment thereof comprises a light chain variable region sequence comprising the amino acid sequence of SEQ ID NO: 1530; or comprises a light chain variable region sequence which is at least 85%, 90%, 95%, 98%, or 99% identical to the amino acid sequence of SEQ ID NO: 1530.

In various embodiments, the antibody or antigen binding fragment thereof comprises a heavy chain variable region sequence comprising the amino acid sequence of SEQ ID NO: 1535; or comprises a heavy chain variable region sequence which is at least 85%, 90%, 95%, 98%, or 99% identical to the amino acid sequence of SEQ ID NO: 1535.

In various embodiments, the antibody or antigen binding fragment thereof comprises a light chain variable region sequence comprising the amino acid sequence of SEQ ID NO: 1540; or comprises a light chain variable region sequence which is at least 85%, 90%, 95%, 98%, or 99% identical to the amino acid sequence of SEQ ID NO: 1540.

In various embodiments, the antibody or antigen binding fragment thereof comprises a heavy chain variable region sequence comprising the amino acid sequence of SEQ ID NO: 1545; or comprises a heavy chain variable region sequence which is at least 85%, 90%, 95%, 98%, or 99% identical to the amino acid sequence of SEQ ID NO: 1545.

In various embodiments, the antibody or antigen binding fragment thereof comprises a light chain variable region sequence comprising the amino acid sequence of SEQ ID NO: 1550; or comprises a light chain variable region sequence which is at least 85%, 90%, 95%, 98%, or 99% identical to the amino acid sequence of SEQ ID NO: 1550.

In various embodiments, the antibody or antigen binding fragment thereof comprises a heavy chain variable region sequence comprising the amino acid sequence of SEQ ID NO: 1555; or comprises a heavy chain variable region sequence which is at least 85%, 90%, 95%, 98%, or 99% identical to the amino acid sequence of SEQ ID NO: 1555.

In various embodiments, the antibody or antigen binding fragment thereof comprises a light chain variable region sequence comprising the amino acid sequence of SEQ ID NO: 1560; or comprises a light chain variable region sequence which is at least 85%, 90%, 95%, 98%, or 99% identical to the amino acid sequence of SEQ ID NO: 1560.

In various embodiments, the antibody or antigen binding fragment thereof comprises heavy and light chain variable region sequences which are selected from the group consisting of: (1) SEQ ID NOs: 1295 and 1300, respectively, (2) SEQ ID NOs: 1305 and 1310, respectively, (3) SEQ ID NOs: 1315 and 1320, respectively; (4) SEQ ID NOs: 1325 and 1330, respectively; (5) SEQ ID NOs: 1335 and 1340, respectively; (6) SEQ ID NOs: 1345 and 1350, respectively; (7) SEQ ID NOs: 1355 and 1360, respectively; (8) SEQ ID NOs: 1365 and 1370, respectively; (9) SEQ ID NOs: 1375 and 1380, respectively; (10) SEQ ID NOs: 1385 and 1390, respectively; (11) SEQ ID NOs: 1395 and 1400, respectively; (12) SEQ ID NOs: 1405 and 1410, respectively; (13) SEQ ID NOs: 1415 and 1420, respectively; (14) SEQ ID NOs: 1425 and 1430, respectively; (15) SEQ ID NOs: 1435 and 1440, respectively; (16) SEQ ID NOs: 1445 and 1450, respectively; (17) SEQ ID NOs: 1455 and 1460, respectively; (18) SEQ ID NOs: 1465 and 1470, respectively; (19) SEQ ID NOs: 1475 and 1480, respectively; (20) SEQ ID NOs: 1485 and 1490, respectively; (21) SEQ ID NOs: 1495 and 1500, respectively; (22) SEQ ID NOs: 1505 and 1510, respectively; (23) SEQ ID NOs: 1515 and 1520, respectively; (24) SEQ ID NOs: 1525 and 1530, respectively; (25) SEQ ID NOs: 1535 and 1540, respectively; (26) SEQ ID NOs: 1545 and 1550, respectively; and (27) SEQ ID NOs: 1555 and 1560, respectively.

In various embodiments, the antibody or antigen binding fragment thereof comprises heavy and light chain variable region sequences which are at least 85%, 90%, 95%, 98%, or 99% identical to the amino acid sequences selected from the group consisting of: (1) SEQ ID NOs: 1295 and 1300, respectively, (2) SEQ ID NOs: 1305 and 1310, respectively, (3) SEQ ID NOs: 1315 and 1320, respectively; (4) SEQ ID NOs: 1325 and 1330, respectively; (5) SEQ ID NOs: 1335 and 1340, respectively; (6) SEQ ID NOs: 1345 and 1350, respectively; (7) SEQ ID NOs: 1355 and 1360, respectively; (8) SEQ ID NOs: 1365 and 1370, respectively; (9) SEQ ID NOs: 1375 and 1380, respectively; (10) SEQ ID NOs: 1385 and 1390, respectively; (11) SEQ ID NOs: 1395 and 1400, respectively; (12) SEQ ID NOs: 1405 and 1410, respectively; (13) SEQ ID NOs: 1415 and 1420, respectively; (14) SEQ ID NOs: 1425 and 1430, respectively; (15) SEQ ID NOs: 1435 and 1440, respectively; (16) SEQ ID NOs: 1445 and 1450, respectively; (17) SEQ ID NOs: 1455 and 1460, respectively; (18) SEQ ID NOs: 1465 and 1470, respectively; (19) SEQ ID NOs: 1475 and 1480, respectively; (20) SEQ ID NOs: 1485 and 1490, respectively; (21) SEQ ID NOs: 1495 and 1500, respectively; (22) SEQ ID NOs: 1505 and 1510, respectively; (23) SEQ ID NOs: 1515 and 1520, respectively; (24) SEQ ID NOs: 1525 and 1530, respectively; (25) SEQ ID NOs: 1535 and 1540, respectively; (26) SEQ ID NOs: 1545 and 1550, respectively; and (27) SEQ ID NOs: 1555 and 1560, respectively.

In various embodiments, the antibody or antigen binding fragment thereof comprises heavy and light chain variable region sequences which are selected from the group consisting of: (1) SEQ ID NOs: 2589 and 2604; (2) SEQ ID NOs:2590 and 2605; (3) SEQ ID NOs: 2591 and 2606; (4) SEQ ID NOs:2592 and 2607; (5) SEQ ID NOs: 2593 and 2608; (6) SEQ ID NOs: 2594 and 2609; (7) SEQ ID NOs: 2595 and 2610; (8) SEQ ID NOs: 2596 and 2611; (9) SEQ ID NOs: 2597 and 2612; (10) SEQ ID NOs: 2598 and 2613; (11) SEQ ID NOs: 2599 and 2614; (12) SEQ ID NOs: 2600 and 2615; (13) SEQ ID NOs: 2601 and 2616; (14) SEQ ID NOs: 2602 and 2617; and (15) SEQ ID NOs: 2603 and 2618.

In various embodiments, the antibody or antigen binding fragment thereof comprises a heavy chain sequence and light chain sequence which is at least 85%, 90%, 95%, 98%, or 99% identical to the amino acid sequences selected from the group consisting of: (1) SEQ ID NOs: 2589 and 2604; (2) SEQ ID NOs:2590 and 2605; (3) SEQ ID NOs: 2591 and 2606; (4) SEQ ID NOs:2592 and 2607; (5) SEQ ID NOs: 2593 and 2608; (6) SEQ ID NOs: 2594 and 2609; (7) SEQ ID NOs: 2595 and 2610; (8) SEQ ID NOs: 2596 and 2611; (9) SEQ ID NOs: 2597 and 2612; (10) SEQ ID NOs: 2598 and 2613; (11) SEQ ID NOs: 2599 and 2614; (12) SEQ ID NOs: 2600 and 2615; (13) SEQ ID NOs: 2601 and 2616; (14) SEQ ID NOs: 2602 and 2617; and (15) SEQ ID NOs: 2603 and 2618.

In various embodiments, the antibody or antigen binding fragment thereof comprises a heavy chain selected from the group consisting of: SEQ ID NOs. 47, 52, 57, 62, 67, 72, 77, 82, 87, and 92; and which comprises a light chain sequence selected from the group consisting of: SEQ ID NOs. 12, 17, 22, 27, 32, 37, and 42.

In various embodiments, the antibody or antigen binding fragment thereof comprises a heavy chain sequence which is at least 85%, 90%, 95%, 98%, or 99% identical to the amino acid sequences selected from the group consisting of: SEQ ID NOs. 47, 52, 57, 62, 67, 72, 77, 82, 87, and 92; and which comprises a light chain sequence which is at least 85%, 90%, 95%, 98%, or 99% identical to the amino acid sequences selected from the group consisting of: SEQ ID NOs. 12, 17, 22, 27, 32, 37, and 42.

In various embodiments, the antibody or antigen binding fragment thereof comprises a heavy chain and light chain selected from the group consisting of: (1) SEQ ID NOs: 99 and 104, respectively, (2) SEQ ID NOs: 109 and 114, respectively, (3) SEQ ID NOs: 119 and 124, respectively; (4) SEQ ID NOs: 129 and 134, respectively; (5) SEQ ID NOs: 139 and 144, respectively; (6) SEQ ID NOs: 149 and 154, respectively; (7) SEQ ID NOs: 159 and 164, respectively; (8) SEQ ID NOs: 169 and 174, respectively; (9) SEQ ID NOs: 179 and 184, respectively; (10) SEQ ID NOs: 189 and 194, respectively; (11) SEQ ID NOs: 199 and 204, respectively; (12) SEQ ID NOs: 209 and 214, respectively; (13) SEQ ID NOs: 219 and 224, respectively; (14) SEQ ID NOs: 229 and 234, respectively; (15) SEQ ID NOs: 239 and 244, respectively; (16) SEQ ID NOs: 249 and 254, respectively; (17) SEQ ID NOs: 259 and 264, respectively; (18) SEQ ID NOs: 269 and 274, respectively; (19) SEQ ID NOs: 279 and 284, respectively; (20) SEQ ID NOs: 289 and 294, respectively; (21) SEQ ID NOs: 299 and 304, respectively; (22) SEQ ID NOs: 309 and 314, respectively; (23) SEQ ID NOs: 319 and 324, respectively; (24) SEQ ID NOs: 329 and 334, respectively; (25) SEQ ID NOs: 339 and 344, respectively; (26) SEQ ID NOs: 349 and 354, respectively; (27) SEQ ID NOs: 359 and 364, respectively; (28) SEQ ID NOs: 369 and 374, respectively; (29) SEQ ID NOs: 379 and 384, respectively; (30) SEQ ID NOs: 389 and 394, respectively; (31) SEQ ID NOs: 399 and 404, respectively; (32) SEQ ID NOs: 409 and 414, respectively; (33) SEQ ID NOs: 419 and 424, respectively; (34) SEQ ID NOs: 429 and 434, respectively; (35) SEQ ID NOs: 439 and 444, respectively; (36) SEQ ID NOs: 459 and 464, respectively; (37) SEQ ID NOs: 469 and 474, respectively; (38) SEQ ID NOs: 479 and 484, respectively; (39) SEQ ID NOs: 489 and 494, respectively; (40) SEQ ID NOs: 499 and 504, respectively; (41) SEQ ID NOs: 509 and 514, respectively; (42) SEQ ID NOs: 519 and 524, respectively; (43) SEQ ID NOs: 529 and 534, respectively; and (44) SEQ ID NOs: 539 and 544, respectively.

In various embodiments, the antibody or antigen binding fragment thereof comprises heavy and light chain sequences which are at least 85%, 90%, 95%, 98%, or 99% identical to the amino acid sequences selected from the group consisting of: (1) SEQ ID NOs: 99 and 104, respectively, (2) SEQ ID NOs: 109 and 114, respectively, (3) SEQ ID NOs: 119 and 124, respectively; (4) SEQ ID NOs: 129 and 134, respectively; (5) SEQ ID NOs: 139 and 144, respectively; (6) SEQ ID NOs: 149 and 154, respectively; (7) SEQ ID NOs: 159 and 164, respectively; (8) SEQ ID NOs: 169 and 174, respectively; (9) SEQ ID NOs: 179 and 184, respectively; (10) SEQ ID NOs: 189 and 194, respectively; (11) SEQ ID NOs: 199 and 204, respectively; (12) SEQ ID NOs: 209 and 214, respectively; (13) SEQ ID NOs: 219 and 224, respectively; (14) SEQ ID NOs: 229 and 234, respectively; (15) SEQ ID NOs: 239 and 244, respectively; (16) SEQ ID NOs: 249 and 254, respectively; (17) SEQ ID NOs: 259 and 264, respectively; (18) SEQ ID NOs: 269 and 274, respectively; (19) SEQ ID NOs: 279 and 284, respectively; (20) SEQ ID NOs: 289 and 294, respectively; (21) SEQ ID NOs: 299 and 304, respectively; (22) SEQ ID NOs: 309 and 314, respectively; (23) SEQ ID NOs: 319 and 324, respectively; (24) SEQ ID NOs: 329 and 334, respectively; (25) SEQ ID NOs: 339 and 344, respectively; (26) SEQ ID NOs: 349 and 354, respectively; (27) SEQ ID NOs: 359 and 364, respectively; (28) SEQ ID NOs: 369 and 374, respectively; (29) SEQ ID NOs: 379 and 384, respectively; (30) SEQ ID NOs: 389 and 394, respectively; (31) SEQ ID NOs: 399 and 404, respectively; (32) SEQ ID NOs: 409 and 414, respectively; (33) SEQ ID NOs: 419 and 424, respectively; (34) SEQ ID NOs: 429 and 434, respectively; (35) SEQ ID NOs: 439 and 444, respectively; (36) SEQ ID NOs: 459 and 464, respectively; (37) SEQ ID NOs: 469 and 474, respectively; (38) SEQ ID NOs: 479 and 484, respectively; (39) SEQ ID NOs: 489 and 494, respectively; (40) SEQ ID NOs: 499 and 504, respectively; (41) SEQ ID NOs: 509 and 514, respectively; (42) SEQ ID NOs: 519 and 524, respectively; (43) SEQ ID NOs: 529 and 534, respectively; and (44) SEQ ID NOs: 539 and 544, respectively.

In various embodiments, the antibody or antigen binding fragment thereof comprises a heavy chain and a light chain selected from the group consisting of: SEQ ID NOs: 549 and 554, respectively, (2) SEQ ID NOs: 559 and 564, respectively, and SEQ ID NOs: 569 and 574, respectively.

In various embodiments, the antibody or antigen binding fragment thereof comprises heavy and light chain variable region sequences which are at least 85%, 90%, 95%, 98%, or 99% identical to the amino acid sequences selected from the group consisting of: SEQ ID NOs: 549 and 554, respectively, (2) SEQ ID NOs: 559 and 564, respectively, and SEQ ID NOs: 569 and 574, respectively.

In various embodiments, the antibody or antigen binding fragment thereof comprises a heavy chain and a light chain selected from the group consisting of: SEQ ID NO: (1) SEQ ID NOs: 1296 and 1301, respectively, (2) SEQ ID NOs: 1306 and 1311, respectively, (3) SEQ ID NOs: 1316 and 1321, respectively; (4) SEQ ID NOs: 1326 and 1331, respectively; (5) SEQ ID NOs: 1336 and 1341, respectively; (6) SEQ ID NOs: 1346 and 1351, respectively; (7) SEQ ID NOs: 1356 and 1361, respectively; (8) SEQ ID NOs: 1366 and 1371, respectively; (9) SEQ ID NOs: 1376 and 1381, respectively; (10) SEQ ID NOs: 1386 and 1391, respectively; (11) SEQ ID NOs: 1396 and 1401, respectively; (12) SEQ ID NOs: 1406 and 1411, respectively; (13) SEQ ID NOs: 1416 and 1421, respectively; (14) SEQ ID NOs: 1426 and 1431, respectively; (15) SEQ ID NOs: 1436 and 1441, respectively; (16) SEQ ID NOs: 1446 and 1451, respectively; (17) SEQ ID NOs: 1456 and 1461, respectively; (18) SEQ ID NOs: 1466 and 1471, respectively; (19) SEQ ID NOs: 1476 and 1481, respectively; (20) SEQ ID NOs: 1486 and 1491, respectively; (21) SEQ ID NOs: 1496 and 1501, respectively; (22) SEQ ID NOs: 1506 and 1511, respectively; (23) SEQ ID NOs: 1516 and 1521, respectively; (24) SEQ ID NOs: 1526 and 1531, respectively; (25) SEQ ID NOs: 1536 and 1541, respectively; (26) SEQ ID NOs: 1546 and 1551, respectively; and (27) SEQ ID NOs: 1556 and 1561, respectively.

In various embodiments, the antibody or antigen binding fragment thereof comprises heavy and light chain variable region sequences which are at least 85%, 90%, 95%, 98%, or 99% identical to the amino acid sequences selected from the group consisting of: (1) SEQ ID NOs: 1296 and 1301, respectively, (2) SEQ ID NOs: 1306 and 1311, respectively, (3) SEQ ID NOs: 1316 and 1321, respectively; (4) SEQ ID NOs: 1326 and 1331, respectively; (5) SEQ ID NOs: 1336 and 1341, respectively; (6) SEQ ID NOs: 1346 and 1351, respectively; (7) SEQ ID NOs: 1356 and 1361, respectively; (8) SEQ ID NOs: 1366 and 1371, respectively; (9) SEQ ID NOs: 1376 and 1381, respectively; (10) SEQ ID NOs: 1386 and 1391, respectively; (11) SEQ ID NOs: 1396 and 1401, respectively; (12) SEQ ID NOs: 1406 and 1411, respectively; (13) SEQ ID NOs: 1416 and 1421, respectively; (14) SEQ ID NOs: 1426 and 1431, respectively; (15) SEQ ID NOs: 1436 and 1441, respectively; (16) SEQ ID NOs: 1446 and 1451, respectively; (17) SEQ ID NOs: 1456 and 1461, respectively; (18) SEQ ID NOs: 1466 and 1471, respectively; (19) SEQ ID NOs: 1476 and 1481, respectively; (20) SEQ ID NOs: 1486 and 1491, respectively; (21) SEQ ID NOs: 1496 and 1501, respectively; (22) SEQ ID NOs: 1506 and 1511, respectively; (23) SEQ ID NOs: 1516 and 1521, respectively; (24) SEQ ID NOs: 1526 and 1531, respectively; (25) SEQ ID NOs: 1536 and 1541, respectively; (26) SEQ ID NOs: 1546 and 1551, respectively; and (27) SEQ ID NOs: 1556 and 1561, respectively.

In various embodiments, the antibody or antigen binding fragment thereof comprises a heavy chain variable region sequence comprising an amino acid sequence of SEQ ID NO: 2217, or a heavy chain variable region sequence which is at least 85%, 90%, 95%, 98%, or 99% identical to the amino acid sequence of SEQ ID NO: 2217.

In various embodiments, the antibody or antigen binding fragment thereof comprises a light chain variable region sequence comprising an amino acid sequence of SEQ ID NO: 2221, or a heavy chain variable region sequence which is at least 85%, 90%, 95%, 98%, or 99% identical to the amino acid sequence of SEQ ID NO: 2221.

In various embodiments, the antibody or antigen binding fragment thereof comprises a heavy chain variable region sequence comprising an amino acid sequence of SEQ ID NO: 2217, and comprises a light chain variable region sequence comprising an amino acid sequence of SEQ ID NO: 2221.

In various embodiments, the antibody or antigen binding fragment thereof comprises a heavy chain and light chain sequences selected from the group consisting of: SEQ ID NOs: 2619 and 2634, respectively, (2) SEQ ID NOs: 2620 and 2635, respectively, (3) SEQ ID NOs: 2621 and 2636, respectively; (4). SEQ ID NOs: 2622 and 2637; (5) SEQ ID NOs: 2623 and 2638; (6) SEQ ID NOs: 2624 and 2639; (7) SEQ ID NOs: 2625 and 2640; (8) SEQ ID NOs: 2626 and 2641; (9) SEQ ID NOs: 2627 and 2642; (10) SEQ ID NOs: 2628 and 2643; (11) SEQ ID NOs: 2629 and 2644; (12) SEQ ID NOs: 2630 and 2645; (13) SEQ ID NOs: 2631 and 2646; (14) SEQ ID NOs: 2632 and 2647; and (15) SEQ ID NOs: 2633 and 2648.

In various embodiments, the antibody or antigen binding fragment thereof comprises heavy and light chain sequences which are at least 85%, 90%, 95%, 98%, or 99% identical to the amino acid sequences selected from the group consisting of: SEQ ID NOs: 2619 and 2634, respectively, (2) SEQ ID NOs: 2620 and 2635, respectively, (3) SEQ ID NOs: 2621 and 2636, respectively; (4). SEQ ID NOs: 2622 and 2637; (5) SEQ ID NOs: 2623 and 2638; (6) SEQ ID NOs: 2624 and 2639; (7) SEQ ID NOs: 2625 and 2640; (8) SEQ ID NOs: 2626 and 2641; (9) SEQ ID NOs: 2627 and 2642; (10) SEQ ID NOs: 2628 and 2643; (11) SEQ ID NOs: 2629 and 2644; (12) SEQ ID NOs: 2630 and 2645; (13) SEQ ID NOs: 2631 and 2646; (14) SEQ ID NOs: 2632 and 2647; and (15) SEQ ID NOs: 2633 and 2648.

In various embodiments, the antibody or antigen binding fragment thereof comprises at least one conservative sequence modification, substitution, or deletion.

In various embodiments, the antibody or antigen binding fragment thereof binds to human LAP.

In various embodiments, the antibody or antigen binding fragment thereof binding to a complex comprising LAP/TGFβ1.

In various embodiments, the antibody or antigen binding fragment inhibits TGFβ1 activation.

In various embodiments, the antibody or antigen binding fragment thereof of any of the preceding claims binds to LAP (e.g., human LAP) and/or to a complex comprising LAP/TGFβ1 with a K_{D} as described herein, for example, in Table 44. In various embodiments, the antibody or antigen binding fragment thereof of any of the preceding claims binds to LAP (e.g., human LAP) and/or to a complex comprising LAP/TGFβ1 with a K_{D} of 30 nanomolar (nM) or less, 20 nM or less, 10 nM or less, 1 nM or less, 0.9 nM or less, 0.8 nM or less, 0.7 nM or less, 0.6 nM or less, 0.5 nM or less, 0.4 nM or less, 0.3 nM or less, 0.2 nM or less, 0.1 nM or less, 0.09 nM or less, 0.08 nM or less, 0.07 nM or less, 0.06 nM or less, 0.05 nM or less, 0.04 nM or less, 0.03 nM or less, 0.02 nM or less, 10 picomolar (pM) or less, 9 pM or less, 8 pM or less, or 7 nM or less.

In various embodiments, the antibody or antigen binding fragment thereof binds to LAP complexed with an anchor protein on immunosuppressive cells, but does not bind to the anchor protein or to an epitope composed of residues of both LAP and the anchor protein. For example, the anchor protein is GARP or LRRC33. In various embodiments, the immunosuppressive cells are regulatory T cells, M2 macrophages, cancer cells expressing LAP, and/or myeloid-derived suppressor cells.

In various embodiments, the antibody or antigen binding fragment binds to both a GARP-positive immunosuppressive cell and a GARP-negative immunosuppressive cell.

In various embodiments, the antibody or antigen binding fragment does not bind to LAP on extracellular matrix.

In various embodiments, the antibody or antigen binding fragment does not bind to LAP complexed with LTBP1, LTBP3 and/or LTBP4.

In various embodiments, the antibody comprises an IgG constant region or variant thereof. For example, the antibody comprises an IgG1 constant domain or variant thereof. In various embodiments, the antibody comprises a constant domain shown in a Table herein. For example, the antibody comprises a human_IgG1_L234A_L235A as described below:

Human IgG1_P01857_L234A_L235A
(SEQ ID NO: 2225)
ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKKVEPKSCDKTHTCPPCPAPEaaGG
PSVFLFPPKPKDTLMISRTPEVTCVVVDVSHEDPEVKFNW
YVDGVEVHNAKTKPREEQYNSTYRVVSVLTVLHQDWLNGK
EYKCKVSNKALPAPIEKTISKAKGQPREPQVYTLPPSRDE
LTKNQVSLTCLVKGFYPSDIAVEWESNGQPENNYKTTPPV
LDSDGSFFLYSKLTVDKSRWQQGNVFSCSVMHEALHNHYT
QKSLSLSPGK

For example, the antibody comprises a human_IgG1_L234A_L235A_D265S as described below:

Human IgG1_P01857_L234A_L235A_D265S
(SEQ ID NO: 2226)
ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKKVEPKSCDKTHTCPPCPAPEaaGG
PSVFLFPPKPKDTLMISRTPEVTCVVVsVSHEDPEVKFNW
YVDGVEVHNAKTKPREEQYNSTYRVVSVLTVLHQDWLNGK
EYKCKVSNKALPAPIEKTISKAKGQPREPQVYTLPPSRDE
LTKNQVSLTCLVKGFYPSDIAVEWESNGQPENNYKTTPPV
LDSDGSFFLYSKLTVDKSRWQQGNVFSCSVMHEALHNHYT
QKSLSLSPGK

In various embodiments, the antibody comprises an IgG4 constant domain or variant thereof. In various embodiments, the antibody comprises a constant domain shown in a Table herein.

In various embodiments, the antibody is a chimeric, human or humanized antibody.

In various embodiments, the antibody or antigen binding fragment thereof binds to the same epitope on LAP as the antibody of any of claims described herein.

In various embodiments, the antibody or antigen binding fragment thereof binds to an epitope of LAP.

An aspect of the invention provides an antibody or antigen binding fragment thereof which binds to one or more residues of residues 31-40, 274-280, and 340-343 of human LAP-TGFβ1 (SEQ ID NO: 1), or binds to one or more residues of residues 31-43, 272-283, and 340-344 of human LAP-TGFβ1 (SEQ ID NO: 1). In various embodiments, the antibody or antigen binding fragment thereof binds to each of the residues 31-40, 274-280, and 340-343 of human LAP-TGFβ1 (SEQ ID NO: 1). In various embodiments, the antibody or antigen binding fragment thereof binds to one of the residues in the range of amino acid residues 31-40, 274-280, and 340-343 of human LAP-TGFβ1 (SEQ ID NO: 1). In various embodiments, the antibody or antigen binding fragment thereof binds to each of the residues 31-43, 272-283, and 340-344 of human LAP-TGFβ1 (SEQ ID NO: 1). In various embodiments, the antibody or antigen binding fragment thereof binds to one of the residues in the range of amino acid residues 31-43, 272-283, and 340-344 of human LAP-TGFβ1 (SEQ ID NO: 1). In various embodiments, the antibody is an isolated antibody or a monoclonal antibody.

An aspect of the invention provides a bispecific molecule comprising the antibody or antigen binding fragment thereof described herein linked to a molecule having a second binding region. In various embodiments, the antibody or antigen binding fragment thereof comprises an amino acid sequence described in Tables 4, 6, 8, 11-43 and 45.

In various embodiments, the second binding region binds to a tumor-associated antigen. For example, the second binding region binds to CD4, CD8, CD45, CD56, CD14, CD16, CD19, CD11b, CD25, CD20, CD22, CD30, CD38, CD114, CD23, CD73, CD163, CD206, CD203, CD200R, or CD39.

An aspect of the invention provides an immunoconjugate comprising the antibody or antigen binding fragment thereof described herein, linked to a detectable moiety, a binding moiety, a labeling moiety, or a biologically active moiety. In various embodiments, the antibody or antigen binding fragment thereof comprises an amino acid sequence described in Tables 4, 6, 8, 11-43 and 45.

An aspect of the invention provides a nucleic acid comprising a nucleotide sequence that encodes the heavy and/or light chain variable region of the antibody or antigen binding fragment thereof described herein. In various embodiments, the antibody or antigen binding fragment thereof comprises an amino acid sequence described in Tables 4, 6, 8, 11-43 and 45. An aspect of the invention provides an expression vector comprising the nucleic acid described herein.

An aspect of the invention provides a cell transformed with an expression vector described herein.

An aspect of the invention provides a pharmaceutical composition comprising the antibody or antigen binding fragment, bispecific molecule, or immunoconjugate described herein, and a pharmaceutically acceptable carrier. In various embodiments, the antibody or antigen binding fragment thereof comprises an amino acid sequence described in Tables 4, 6, 8, 11-43 and 45.

In various embodiments, the pharmaceutical composition further comprises one or more additional therapeutic agents. In various embodiments, the one or more additional therapeutic agents is selected from the group consisting of an anti-cancer agent, a chemotherapeutic agent, an immunosuppressive agent, an immunostimulatory agent, an anti-inflammatory agent, and an immune checkpoint inhibitor.

In various embodiments, the pharmaceutical composition further comprises an agent selected from the group consisting of:
a. an anti-PD1 antibody or an antigen binding fragment thereof,
b. an anti-LAG3 antibody or an antigen binding fragment thereof,
c. an anti-VISTA antibody or an antigen binding fragment thereof,
d. an anti-BTLA antibody or an antigen binding fragment thereof,
e. an anti-TIM3 antibody or an antigen binding fragment thereof,
f. an anti-CTLA4 antibody or an antigen binding fragment thereof,
g. an anti-HVEM antibody or an antigen binding fragment thereof,
h. an anti-CD27 antibody or an antigen binding fragment thereof,
i. an anti-CD137 antibody or an antigen binding fragment thereof,
j. an anti-OX40 antibody or an antigen binding fragment thereof,
k. an anti-CD28 antibody or an antigen binding fragment thereof,
l. an anti-PDL1 antibody or an antigen binding fragment thereof,
m. an anti-PDL2 antibody or an antigen binding fragment thereof,
n. an anti-GITR antibody or an antigen binding fragment thereof,
o. an anti-ICOS antibody or an antigen binding fragment thereof,
p. an anti-SIRPα antibody or an antigen binding fragment thereof,
q. an anti-ILT2 antibody or an antigen binding fragment thereof,
r. an anti-ILT3 antibody or an antigen binding fragment thereof,
s. an anti-ILT4 antibody or an antigen binding fragment thereof, and
t. an anti-ILT5 antibody or an antigen binding fragment thereof.

In various embodiments of the pharmaceutical composition, the anti-PD1 antibody or an antigen binding fragment thereof is selected from the group consisting of: pembrolizumab or an antigen binding fragment thereof and nivolumab or an antigen binding fragment thereof. For example, the anti-PD1 antibody is pembrolizumab. In various embodiments, the anti-PD-1 antibody is pembrolizumab comprising a heavy chain comprising the amino acid sequence of SEQ ID NO: 2213 and a light chain comprising the amino acid sequence of SEQ ID NO: 2214. In various embodiments, the anti-PD1 antibody is nivolumab. In various embodiments, the anti-PD-1 antibody is nivolumab comprising a heavy chain comprising the amino acid sequence of SEQ ID NO: 2215 and a light chain comprising the amino acid sequence of SEQ ID NO: 2216.

An aspect of the invention provides a kit comprising the antibody or antigen binding fragment thereof, bispecific molecule, or immunoconjugate described herein, and instructions for use. In various embodiments, the antibody or antigen binding fragment thereof comprises an amino acid sequence described in Tables 4, 6, 8, 11-43 and 45.

An aspect of the invention provides a method of producing an antibody or antigen binding fragment thereof comprising: culturing a host cell comprising a polynucleotide encoding the amino acid sequences of any one of the antibodies or antigen binding fragments described herein under conditions favorable to expression of the polynucleotide; and optionally, recovering the antibody or antigen binding fragment thereof from the host cell and/or culture medium. In various embodiments, the antibody or antigen binding fragment thereof comprises an amino acid sequence described herein, for example, in Tables 4, 6, 8, 11-43 and 45.

An aspect of the invention provides a method of selectively inhibiting TGFβ1 activation on cells expressing LAP, but not inhibiting TGFβ1 activation on extracellular matrix, comprising administering to a subject a therapeutically effective amount of the antibody or antigen binding fragment thereof, bispecific molecule, the immunoconjugate, or the pharmaceutical composition described herein. In various embodiments, the antibody or antigen binding fragment thereof comprises an amino acid sequence described herein, for example, in Tables 4, 6, 8, 11-43 and 45.

In various embodiments of the method, the cell is an immunosuppressive cell. In various embodiments, the immunosuppressive cell is selected from the group consisting of suppressive T cells, M2 macrophages, cancer cells expressing LAP-TGFβ1, and monocytic myeloid-derived suppressor cells.

An aspect of the invention provides a method of treating cancer comprising administering to a subject in need thereof a therapeutically effective amount the antibody or antigen binding fragment described herein. In various embodiments, the antibody or antigen binding fragment thereof comprises an amino acid sequence described herein, for example, in Tables 4, 6, 8, 11-43 and 45.

An aspect of the invention provides a method of treating cancer comprising administering to a subject in need thereof the antibody or antigen binding fragment thereof, the bispecific molecule, the immunoconjugate, or the pharmaceutical composition described herein. In various embodiments, the antibody or antigen binding fragment thereof comprises an amino acid sequence described herein, for example, in Tables 4, 6, 8, 11-43 and 45.

In various embodiments of the method, the cancer is characterized by abnormal TGFβ activity. In various embodiments of the method, the cancer is associated with infiltration of CD4+ regulatory T cells, CD8+ regulatory T cells, regulatory B cells, myeloid-derived suppressor cells, tumor-associated macrophages, cancer-associated fibroblasts, and/or innate lymphoid cells. In various embodiments of the method, the cancer is selected from the group consisting of: breast cancer, bladder cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and myelodysplastic syndromes.

In various embodiments, the method further comprises administering one or more additional therapies. For example, the one or more additional therapies is selected from radiation therapy, chemotherapy, an immune checkpoint inhibitor, immunosuppressive therapy, immunostimulatory therapy, cell therapy, and a therapeutic agent.

In various embodiments, the method further comprises administering an agent selected from the group consisting of:
a. an anti-PD1 antibody or an antigen binding fragment thereof,
b. an anti-LAG3 antibody or an antigen binding fragment thereof,
c. an anti-VISTA antibody or an antigen binding fragment thereof,
d. an anti-BTLA antibody or an antigen binding fragment thereof,
e. an anti-TIM3 antibody or an antigen binding fragment thereof,
f. an anti-CTLA4 antibody or an antigen binding fragment thereof,
g. an anti-HVEM antibody or an antigen binding fragment thereof,
h. an anti-CD27 antibody or an antigen binding fragment thereof,
i. an anti-CD137 antibody or an antigen binding fragment thereof,
j. an anti-OX40 antibody or an antigen binding fragment thereof,
k. an anti-CD28 antibody or an antigen binding fragment thereof,
l. an anti-PDL1 antibody or an antigen binding fragment thereof,
m. an anti-PDL2 antibody or an antigen binding fragment thereof,
n. an anti-GITR antibody or an antigen binding fragment thereof,
o. an anti-ICOS antibody or an antigen binding fragment thereof,
p. an anti-SIRPα antibody or an antigen binding fragment thereof,
q. an anti-ILT2 antibody or an antigen binding fragment thereof,
r. an anti-ILT3 antibody or an antigen binding fragment thereof,
s. an anti-ILT4 antibody or an antigen binding fragment thereof, and
t. an anti-ILT5 antibody or an antigen binding fragment thereof.

An aspect of the invention provides an antibody or antigen binding fragment thereof, the bispecific molecule, the immunoconjugate, or the pharmaceutical composition described herein, for use in the preparation of a medicament to:
increase immune cell activation;
treat cancer; or
treat an infection or infectious disease.

In various embodiments, the antibody or antigen binding fragment thereof comprises an amino acid sequence described herein, for example, in Tables 4, 6, 8, 11-43 and 45.

An aspect of the invention provides use of the antibody or antigen binding fragment thereof, the bispecific molecule, the immunoconjugate, or the pharmaceutical composition described herein for the manufacture of a medicament for: increasing immune cell activation; treating cancer; or treating an infection or infectious disease. In various embodiments, the antibody or antigen binding fragment thereof comprises an amino acid sequence described herein, for example, in Tables 4, 6, 8, 11-43 and 45.

An aspect of the invention provides a method of detecting the presence of LAP in a sample comprising contacting the sample with the antibody or antigen binding fragment thereof described herein, under conditions that allow for formation of a complex between the antibody and LAP, and detecting the formation of a complex. In various embodiments, the antibody or antigen binding fragment thereof comprises an amino acid sequence described herein, for example, in Tables 4, 6, 8, 11-43 and 45.

An aspect of the invention provides a method of diagnosing a cancer associated comprising contacting a biological sample from a patient afflicted with the cancer with the antibody or antigen binding fragment thereof described herein, wherein positive staining with the antibody indicates the cancer is associated with regulatory T cell infiltration. In various embodiments, the antibody or antigen binding fragment thereof comprises an amino acid sequence described herein, for example, in Tables 4, 6, 8, 11-43 and 45.

An aspect of the invention provides a method of diagnosing a cancer associated with GARP-negative suppressive cells comprising contacting a biological sample from a patient afflicted with the cancer with the antibody or antigen binding fragment thereof described herein, wherein positive staining with the antibody and negative staining with an anti-GARP antibody indicates the cancer is associated with GARP-negative suppressive cells. In various embodiments, the antibody or antigen binding fragment thereof comprises an amino acid sequence described herein, for example, in Tables 4, 6, 8, 11-43 and 45.

An aspect of the invention provides a method of selecting a patient afflicted with cancer for treatment with the antibody or antigen binding fragment thereof described herein, comprising contacting a biological sample from the patient with the antibody or antigen binding fragment, wherein positive staining with the antibody indicates the cancer is amenable to treatment with the antibody. In various embodiments, the antibody or antigen binding fragment thereof comprises an amino acid sequence described herein, for example, in Tables 4, 6, 8, 11-43 and 45.

An aspect of the invention provides a method of determining the response of a patient afflicted with cancer to treatment with the antibody or antigen binding fragment thereof described herein, comprising contacting a biological sample from the patient with the antibody or antigen binding fragment, wherein reduced staining with the antibody indicates the cancer is responding to treatment with the antibody. In various embodiments, the antibody or antigen binding fragment thereof comprises an amino acid sequence described herein, for example, in Tables 4, 6, 8, 11-43 and 45.

### BRIEF DESCRIPTION OF THE FIGURES

FIG. 1 is an amino acid sequence alignment showing the 20E6 tyrosine scanning variant designs. The 20E6 VL and VH sequences are shown in alignment with the 20E6 hybridoma antibody sequences and the human germline sequences IGKV1-33*01/IGJK2 and IGHG1-2*05/IGHJ4 used to humanize 20E6, respectively. The residue numbers shown on the top follow the Kabat numbering system and the CDR sequences are highlighted in bold and numbers underlined. The line ‘Tyrosine scan’ shows the CDR positions where each marked residue was changed into a Tyrosine as indicated by ‘Y’. Figure discloses SEQ ID NOS 2650-2651, 333, 2652-2653 and 98, respectively, in order of appearance.

FIG. 2 is an amino acid sequence alignment showing the 20E6 affinity maturation doping library designs. 20E6 VL and VH sequences are shown in alignment with the 20E6 hybridoma antibody sequences and the human germline sequences IGKV1-33*01/IGJK2 and IGHG1-2*05/IGHJ4 used to humanize 20E6, respectively. The residue numbers shown on the top follow the Kabat numbering system and the CDR sequences are highlighted in bold and numbers underlined. CDR residues that are within 5 Angstroms of LAP-TGFb1 in an Ag/Ab complex as shown by cryo-EM studies are identified by dashes. CDR residues that are chosen for sequence diversification by a 79-7-7-7 doping strategy during DNA synthesis are identified in the line ‘To diversify’ by the letter ‘Z’. The number 11 under LCDR2 indicates a paired sequence toggle between ET and HS in the library at equal ratio. Figure discloses SEQ ID NOS 2650-2651, 333, 2652-2653 and 98, respectively, in order of appearance.

FIG. 3 is an amino acid sequence alignment showing the 20E6 affinity maturation doping library designs. 20E6 VL and VH sequences are shown in alignment with the 20E6 hybridoma antibody sequences and the human germline sequences IGKV1-33*01/IGJK2 and IGHG1-2*05/IGHJ4 used to humanize 20E6, respectively. The residue numbers shown on the top follow the Kabat numbering system and the CDR sequences are highlighted in bold and numbers underlined. CDR residues that are within 5 Angstroms of LAP-TGFb1 in an Ag/Ab complex as shown by cryo-EM studies are identified by dashes. CDR residues that are chosen for sequence diversification by a 79-7-7-7 doping strategy during DNA synthesis are identified in the line ‘To diversify’ by the letter ‘Z’. CDR residues that are chosen for sequence diversification by synthesizing as 49% parental amino acid residue and 3% each of 17 A.A. (excluding M, C, W) using the TRIM technology are identified by the letter ‘X’. Figure discloses SEQ ID NOS 2650-2651, 333, 2652-2653 and 98, respectively, in order of appearance.

FIG. 4 A is a set visualization of the 20E6 Doping Library and sorting gates P4, P3, P2, and P1 after two-hour competition, and FIG. 4 B is a visualization of the sorting gate P1 after twelve-hour competition to obtain a wide range of improved affinities. APC: allophycocyanin, PE: phycoerythrin. APC-A=20E6 Doping Library expression on the yeast cell surface. PE-A=binding of 20E6 Doping Library to LAP/TGFb1-Fc.

FIG. 5 A is a visualization of the 20E6 TRIM Library and sorting gates P4, P3, P2, P1 after two-hour competition, and FIG. 5 B is a visualization of the sorting gate P1 after twelve-hour competition to obtain a wide range of improved affinities. APC: allophycocyanin, PE: phycoerythrin. APC-A=20E6 TRIM Library expression on the yeast cell surface. PE-A=binding of 20E6 TRIM Library to LAP/TGFb1-Fc FIG. 6 is a table with drawings of VH CDR sequence logos of 20E6 variants from the five different doping library outputs after the last round of off-rate selection. The numbers of sequences used to generate the sequence logos are shown below the sorting gates. The CDR residue numbers shown on the left are based on the Kabat numbering scheme.

FIG. 7 is a table with drawings of VL CDR sequence logos of 20E6 variants from the five different doping library outputs after the last round of off-rate selection. The numbers of sequences used to generate the sequence logos are shown below the sorting gates. The CDR residue numbers shown on the left are based on the Kabat numbering scheme.

FIG. 8 is a table with drawings of VH CDR sequence logos of 20E6 variants from the five different TRIM library outputs after the last round of off-rate selection. The numbers of sequences used to generate the sequence logos are shown below the sorting gates. The CDR residue numbers shown on the left are based on the Kabat numbering scheme.

FIG. 9 is a table with drawings of VL CDR sequence logos of 20E6 variants from the five different TRIM library outputs after the last round of off-rate selection. The numbers of sequences used to generate the sequence logos are shown below the sorting gates. The CDR residue numbers shown on the left are based on the Kabat numbering scheme.

FIG. 10 is a table with drawings of sequence logos of 432 VH and 457 VL CDRs from all doping and TRIM library selection outputs. The CDR residue numbers shown are based on the Kabat numbering scheme.

FIG. 11 is an affinity plot showing the association (kon) and dissociation (koff) relationships of 20E6 (♦) and variants (●) binding to human LAP-TGFβ1. The calculated KD, the equilibrium dissociation constant, is generated by dividing the koff/kon and results in a value with molar (M) units.

FIG. 12 is a binding response plot showing the observed constant binding partner (CBP) equilibrium response for each equilibrium series, 5 pM CBP (♦) and 100 pM CBP (●) and the fitted equilibrium curve for each series, 5 pM CBP ( [symbol] ) and 100 pM CBP ( [symbol] ).

FIG. 13 is a graph showing the inhibitory effects of parental 20E5 and selected affinity matured humanized antibodies on TGFβ1 activation in P3U1 cells expressing human LAP-TGFβ1.

FIG. 14 is a graph showing binding of anti-LAP/TGFβ1 parental humanized antibody and selected antibody candidates to tumor infiltrating immune cells. Fresh human tumors (8 kidneys and 1 lung) were dissociated and stained with either AF647-labeled isotype control antibody or one of seven AF647-labeled anti-LAP/TGFβ1 antibody candidates at the indicated concentrations. The samples were also stained with a cocktail of antibodies specific for various cell lineage markers to enable gating and identification of various immune populations by flow cytometry. Samples were analyzed using a Becton Dickinson Fortessa flow cytometer and data were analyzed using Flow Jo V10 software. Bars indicate average LAP+ cells expressed as a percent of intra-tumoral monocyte macrophages and error bars represent standard error of the average.

FIG. 15A, FIG. 15B, FIG. 15C, FIG. 15D, FIG. 15E, and FIG. 15F are a series of graphs showing the extent of binding to human RBCs, platelets and WBCs of different antibodies: affinity matured LAP-TGFB antibodies, parental 20E6 antibody, anti-RSV antibody, anti-integrin very late antigen (VLA)-4 antibody and anti-Her2 antibody. FIG. 15A is a graph showing percent AF647+ human RBCs from 2 donors after incubation with 10, 2, 0.2 and 0.02 ug/mL of labeled anti-LAP-TGFB antibodies, parental 20E6 antibody, anti-RSV antibody, anti-VLA-4 antibody and anti-Her2 antibody. FIG. 15B is a graph showing percent AF647+ human platelets from 2 donors after incubation with 10, 2, 0.2 and 0.02 ug/mL of labeled anti-LAP-TGFB antibodies, parental 20E6 antibody, anti-RSV antibody, anti-VLA-4 antibody and anti-Her2 antibody. FIG. 15C is a graph showing binding of 10 ug/mL of labeled anti-LAP-TGFB antibodies, parental 20E6 antibody, anti-RSV antibody, anti-VLA-4 antibody and anti-Her2 antibody (as % AF647+) to human CD20+ B cells. FIG. 15D is a graph showing binding of 10 ug/mL of labeled anti-LAP-TGFB antibodies, parental 20E6 antibody, anti-RSV antibody, anti-VLA-4 antibody and anti-Her2 antibody (as % AF647+) to human CD4+T and CD8+T cells. FIG. 15E is a graph showing binding of 10 ug/mL of labeled anti-LAP-TGFB antibodies, parental 20E6 antibody, anti-RSV antibody, anti-VLA-4 antibody and anti-Her2 antibody (as % AF647+) to human CD14+ monocytes. FIG. 15F is a graph showing binding of 10 ug/mL of labeled anti-LAP-TGFB antibodies, parental 20E6 antibody, anti-RSV antibody, anti-VLA-4 antibody and anti-Her2 antibody (as % AF647+) to human CD56+NK and granulocytes. Anti-VLA-4 antibody was utilized as a positive control antibody in FIG. 15C, FIG. 15D, FIG. 15E, and FIG. 15F.

FIG. 16A, FIG. 16B, FIG. 16C, FIG. 16D, FIG. 16E, and FIG. 16F are a series of graphs showing the extent of binding to rhesus RBCs, platelets and WBCs of different antibodies: affinity matured LAP-TGFB antibodies, parental 20E6 antibody, anti-RSV antibody, anti-VLA-4 antibody and anti-Her2 antibody. FIG. 16A is a graph showing percent AF647+ rhesus RBCs from 2 donors after incubation with 10, 2, 0.2 and 0.02 ug/mL of labeled anti-LAP-TGFB antibodies, parental 20E6 antibody, anti-RSV antibody, anti-VLA-4 antibody and anti-Her2 antibody. FIG. 16B is a graph showing percent AF647+ rhesus RBCs from 2 donors after incubation with 10, 2, 0.2 and 0.02 ug/mL of labeled anti-LAP-TGFB antibodies, parental 20E6 antibody, anti-RSV antibody, anti-VLA-4 antibody and anti-Her2 antibody. FIG. 16C is a graph showing binding of 10 ug/mL of labeled anti-LAP-TGFB antibodies, parental 20E6 antibody, anti-RSV antibody, anti-VLA-4 antibody and anti-Her2 antibody (as % AF647+) to rhesus CD20+ B cells. FIG. 16D is a graph showing binding of 10 ug/mL of labeled anti-LAP-TGFB antibodies, parental 20E6 antibody, anti-RSV antibody, anti-VLA-4 antibody and anti-Her2 antibody (as % AF647+) to rhesus CD4+T and CD8+T cells. FIG. 16E is a graph showing binding of 10 ug/mL of labeled anti-LAP-TGFB antibodies, parental 20E6 antibody, anti-RSV antibody, anti-VLA-4 antibody and anti-Her2 antibody (as % AF647+) to rhesus CD14+ monocytes. FIG. 16F is a graph showing binding of 10 ug/mL of labeled anti-LAP-TGFB antibodies, parental 20E6 antibody, anti-RSV antibody, anti-VLA-4 antibody and anti-Her2 antibody (as % AF647+) to rhesus NKG2A+NK and granulocytes. Natalizumab was utilized as a positive control antibody in FIG. 16C, FIG. 16D, FIG. 16E, and FIG. 16F.

FIG. 17A and FIG. 17B a series of graphs showing the extent of binding to hepatocytes treated with antibodies: affinity matured LAP-TGFB antibodies, parental 20E6 antibody, anti-RSV antibody, anti-VLA-4 antibody and anti-Her2 antibody. FIG. 17A is a graph showing percent AF647+ primary hepatocytes from 2 donors after incubation with 10 ug/mL of labeled anti-LAP-TGFB antibodies, parental 20E6 antibody, anti-RSV antibody, anti-VLA-4 antibody and anti-Her2 antibody compared to untreated control (APC FMO). FIG. 17B is a graph showing the mean fluorescent intensity (MFI) of AF647+ hepatocytes from 2 donors after incubation with 10 ug/mL of labeled anti-LAP-TGFB antibodies, parental 20E6 antibody, anti-RSV antibody, anti-VLA-4 antibody and anti-Her2 antibody compared to untreated allophycocyanin fluorescence-minus one (APC FMO) control.

FIG. 18A and FIG. 18B a series of graphs showing the extent of binding to hepatocytes treated with different antibodies: affinity matured LAP-TGFB antibodies, parental 20E6 antibody, anti-RSV antibody, anti-VLA-4 antibody and anti-Her2 antibody. FIG. 18A is a graph showing percent AF647+ primary keratinocytes from 4 donors after incubation with 10 ug/mL of labeled anti-LAP-TGFB antibodies, parental 20E6 antibody, anti-RSV antibody, anti-VLA-4 antibody and anti-Her2 antibody compared to untreated control (APC FMO). FIG. 18B is a graph showing the mean fluorescent intensity (MFI) of AF647+ keratinocytes from 4 donors after incubation with 10 ug/mL of labeled anti-LAP-TGFB antibodies, parental 20E6 antibody, anti-RSV antibody, anti-VLA-4 antibody and anti-Her2 antibody compared to untreated APC FMO control.

FIG. 19A is a graph showing percent of CD62P⁺ platelet activation for cells treated with different antibodies: affinity matured LAP-TGFB antibodies, parental 20E6 antibody, anti-RSV antibody, anti-VLA-4 antibody and anti-Her2 antibody. FIG. 19B is a graph showing percent of CD62P⁺ platelet activation of with anti-ADP antibody, anti-TRAP-6 antibody, anti-CD9 antibody, and anti-CD151 antibody.

FIG. 20A is a graph showing percent of PAC1⁺ platelet activation for cells treated with different antibodies: affinity matured LAP-TGFB antibodies, parental 20E6 antibody, anti-RSV antibody, anti-VLA-4 antibody and anti-Her2 antibody. FIG. 20B is a graph showing percent of PAC1⁺ platelet activation of with anti-ADP antibody, anti-TRAP-6 antibody, anti-CD9 antibody, and anti-CD151 antibody.

FIG. 21 is an affinity plot showing the association (kₒₙ) and dissociation (k_{off}) relationships of parental humanized 20E6 antibody (♦) and the yeast display mutants (●) binding to human LAP-TGFβ isoform 1. The calculated K_{D}, the equilibrium dissociation constant, was generated by dividing the k_{off}/kₒₙ and results in a value with molar (M) units.

FIG. 22A and FIG. 22B are graphs showing epitope binning for human LAP-TGFβ1 binding yeast display mutants 74BLH and 81BLH, respectively.

### DETAILED DESCRIPTION

### Definitions

“Abnormal” in the context of the activity, level or expression of a molecule means that the activity, level or expression is outside of the normal activity, level or expression for that molecule. “Normal” in the context of activity, level or expression refers to the range of activity, level or expression of the protein found in a population of healthy, gender- and age-matched subjects. The minimal size of this healthy population may be determined using standard statistical measures, e.g., the practitioner could take into account the incidence of the disease in the general population and the level of statistical certainty desired in the results.

As used herein, “Latency associated peptide” or “LAP” refers to the amino-terminal domain of the human TGFβ1 precursor peptide and has the amino acid sequence set forth in SEQ ID NO: 1562. “LAP-TGFβ1” and “LAP/TGFβ1” are used interchangeably herein to refer to the human TGFβ1 precursor peptide (which includes the TGFβ1 cytokine) and includes the amino acid sequence of SEQ ID NO: 1563 (see Uniprot sp|P01137|TGFB1_HUMAN with signal sequence removed).

human LAP-TGFB1
(SEQ ID NO: 1562)
LSTCKTIDMELVKRKRIEAIRGQILSKLRLASPPSQGEVP
PGPLPEAVLALYNSTRDRVAGESAEPEPEPEADYYAKEVT
RVLMVETHNEIYDKFKQSTHSIYMFFNTSELREAVPEPVL
LSRAELRLLRLKLKVEQHVELYQKYSNNSWRYLSNRLLAP
SDSPEWLSFDVTGVVRQWLSRGGEIEGFRLSAHCSCDSRD
NTLQVDINGFTTGRRGDLATIHGMNRPFLLLMATPLERAQ
HLQSSRHRRALDTNYCFSSTEKNCCVRQLYIDFRKDLGWK
WIHEPKGYHANFCLGPCPYIWSLDTQYSKVLALYNQHNPG
ASAAPCCVPQALEPLPIVYYVGRKPKVEQLSNMIVRSCKC
S
LAP region of human LAP-TGFB1
(SEQ ID NO: 1563)
LSTCKTIDMELVKRKRIEAIRGQILSKLRLASPPSQGEVP
PGPLPEAVLALYNSTRDRVAGESAEPEPEPEADYYAKEVT
RVLMVETHNEIYDKFKQSTHSIYMFFNTSELREAVPEPVL
LSRAELRLLRLKLKVEQHVELYQKYSNNSWRYLSNRLLAP
SDSPEWLSFDVTGVVRQWLSRGGEIEGFRLSAHCSCDSRD
NTLQVDINGFTTGRRGDLATIHGMNRPFLLLMATPLERAQ
HLQSSRHRR
human free TGFB1 (mature TGFB1 without LAP
(SEQ ID NO: 1564)
ALDTNYCFSSTEKNCCVRQLYIDFRKDLGWKWIHEPKGYH
ANFCLGPCPYIWSLDTQYSKVLALYNQHNPGASAAPCCVP
QALEPLPIVYYVGRKPKVEQLSNMIVRSCKCS

LAP can also refer to the amino-terminal domains of the human TGFβ2 precursor peptide and human TGFβ3 precursor peptide, as well as their counterparts from other species (e.g., mouse TGFβ1 precursor peptide and mouse LAP-TGFβ1: SEQ ID NO: 7, mouse TGFβ2 precursor peptide, and mouse TGFβ3 precursor peptide) and other naturally occurring allelic, splice variants, and processed forms thereof. See each of WO/2020/076969, WO/2016/115345 and WO/2019/075090, which are incorporated by reference in their entirety.

LAP is synthesized as a complex with TGFβ. LAP in the absence of mature TGFβ is referred to as “empty LAP.” Unless otherwise specified, “empty LAP” as used herein refers to LAP originating from the N-terminal domain of human TGFβ1. In addition to residues on LAP, the anti-LAP antibodies described herein may also bind to residues of mature TGFβ within the LAP-TGFβ1 complex. Notwithstanding, in all cases, the antibody at least binds to residues in the LAP portion of the LAP-TGFβ complex.

As used herein “free TGFβ1” refers to the mature TGFβ1 cytokine, i.e., TGFβ1 that is not complexed with LAP.

As used herein, “anchor protein” refers to a protein that anchors LAP-TGFβ to a cell surface or to the extracellular matrix. Exemplary anchor proteins include GARP, LRRC33, LTBP1, LTBP3, and LTBP4. GARP and LRRC33 are proteins that anchor LAP-TGFβ to the surface of cells, and LTBP1, LTBP3, and LTBP4 are proteins that anchor LAP-TGFβ to the extracellular matrix.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

As used herein, “isotype” refers to the antibody class (e.g., IgG1, IgG2, IgG3, IgG4, IgM, IgA1, IgA2, IgD, and IgE antibody) that is encoded by the heavy chain constant region genes.

Antibodies typically bind specifically to their cognate antigen with high affinity, reflected by a dissociation constant (K_{D}) of 10⁻⁵ to 10⁻¹² M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸M or less, even more preferably 5×10⁻⁹ M or less, and most preferably between 10⁻⁸ M and 10⁻¹⁰M or less, but does not bind with high affinity to unrelated antigens.

As used herein, unless otherwise indicated, “antibody fragment” or “antigen binding fragment” or “antigen binding fragment thereof” refers to a fragment of an antibody that retains the ability to bind specifically to the antigen, e.g., fragments that retain one or more CDR regions. An antibody that “specifically binds to” PD-1, LAG3, or TIGIT is an antibody that exhibits preferential binding to PD-1, LAG3, or TIGIT (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antibody (e.g., PD-1, LAG3, or TIGIT). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

Antibody fragments within the scope of the present invention also include F(ab′)2 fragments which may be produced by enzymatic cleavage of an IgG by, for example, pepsin. Fab fragments may be produced by, for example, reduction of F(ab′)2 with dithiothreitol or mercaptoethylamine. A Fab fragment is a VL-CL chain appended to a VH-CH1 chain by a disulfide bridge. A F(ab′)2 fragment is two Fab fragments which, in turn, are appended by two disulfide bridges. The Fab portion of an F(ab′)2 molecule includes a portion of the Fc region between which disulfide bridges are located.

The term “acceptor human framework” refers to a framework comprising the amino acid sequence of a light chain variable domain (VL) framework or a heavy chain variable domain (VH) framework derived from a human immunoglobulin framework or a human consensus framework. An acceptor human framework “derived from” a human immunoglobulin framework or a human consensus framework may have the same amino acid sequence as the naturally-occurring human immunoglobulin framework or human consensus framework, or it may have amino acid sequence changes compared to wild-type naturally-occurring human immunoglobulin framework or human consensus framework. In some embodiments, the number of amino acid changes are 10, 9, 8, 7, 6, 5, 4, 3, or 2, or 1. In some embodiments, the VL acceptor human framework is identical in sequence to the VL human immunoglobulin framework sequence or human consensus framework sequence.

A “multispecific antibody” is an antibody (e.g., bispecific antibodies, tri-specific antibodies) that recognizes two or more different antigens or epitopes.

The term “binding protein” as used herein also refers to a non-naturally occurring (or recombinant) protein that specifically binds to at least one target antigen.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). Bifunctional antibodies include, for example, heterodimeric antibody conjugates (e.g., two antibodies or antibody fragments joined together with each having different specificities), antibody/cell surface-binding molecule conjugates (e.g., an antibody conjugated to a non-antibody molecule such as a receptor), and hybrid antibodies (e.g., an antibody having binding sites for two different antigens).

The term “recombinant antibody,” refers to antibodies that are prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain embodiments, however, such recombinant human antibodies can be subjected to in vitro mutagenesis and thus the amino acid sequences of the VH and VL regions of the recombinant antibodies are sequences that, while derived from and related to human germline VH and VL sequences, may not naturally exist within the human antibody germline repertoire in vivo.

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

Antigen binding fragments (including scFvs) of such immunoglobulins are also encompassed by the term “monoclonal antibody” as used herein. Monoclonal antibodies are highly specific, being directed against a single antigenic site. Furthermore, in contrast to conventional (polyclonal) antibody preparations, which typically include different antibodies directed against different epitopes on the antigen, each monoclonal antibody is directed against a single epitope. Monoclonal antibodies can be prepared using any art recognized technique and those described herein such as, for example, a hybridoma method, a transgenic animal, recombinant DNA methods (see, e.g., U.S. Pat. No. 4,816,567), or using phage antibody libraries using the techniques described in, for example, U.S. Pat. No. 7,388,088 and PCT Pub. No. WO 00/31246). Monoclonal antibodies include chimeric antibodies, human antibodies, and humanized antibodies and may occur naturally or be produced recombinantly.

A “domain antibody” is an immunologically functional immunoglobulin fragment containing only the variable region of a heavy chain or the variable region of a light chain. In some instances, two or more VH regions are covalently joined with a peptide linker to create a bivalent domain antibody. The two VH regions of a bivalent domain antibody may target the same or different antigens.

A “bivalent antibody” comprises two antigen binding sites. In some instances, the two binding sites have the same antigen specificities. However, bivalent antibodies may be bispecific (see below).

As used herein, the term “single-chain Fv” or “scFv” antibody refers to antibody fragments comprising the VH and VL domains of antibody, wherein these domains are present in a single polypeptide chain. Generally, the Fv polypeptide further comprises a polypeptide linker. For a review of scFv, see Pluckthun (1994) THE PHARMACOLOGY OF MONOCLONAL ANTIBODIES, vol. 113, Rosenburg and Moore eds. Springer-Verlag, New York, pp. 269-315.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079, which are hereby incorporated by reference in their entireties). In one embodiment, the present invention provides single domain antibodies comprising two VH domains with modifications such that single domain antibodies are formed.

As used herein, the term “diabodies” refers to small antibody fragments with two antigen binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

The antibodies of the present invention also include antibodies with modified (or blocked) Fc regions to provide altered effector functions. See, e.g., U.S. Pat. No. 5,624,821; WO2003/086310; WO2005/120571; WO2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes, such as substitutions, deletions and insertions, glycosylation or deglycosylation, and adding multiple Fc. Changes to the Fc may be utilized to alter the half-life of antibodies in therapeutic antibodies, and a longer half-life would result in less frequent dosing, with the concomitant increased convenience and decreased use of material. See Presta (2005) J. Allergy Clin. Immunol. 116:731 at 734-35.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody which comprises mouse immunoglobulin sequences only.

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may also be referred to as “heavy chain variable region”, “heavy chain variable domain”, “VH” or “V_{H}” in the instant disclosure. The variable region of the light chain may be referred to as “light chain variable region”, “heavy chain variable domain”, “VL” or “V_{L}” in the instant disclosure. Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody VH β-sheet framework, or one of three hypervariable regions (L1, L2, or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (A1-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (A1-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system. See Kabat et al., (1991) Sequences of Proteins of Immunological Interest, 5th Ed. Public Health Service, National Institutes of Health, Bethesda, Md.) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, (1987) J. Mol. Biol. 196: 901-917).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG₁ (Eu), which has a constant domain that begins at amino acid position 118 of the amino acid sequence of the IgG₁ described in Edelman et al., Proc. Natl. Acad. Sci. USA. 63: 78-85 (1969), and is shown for the IgG₁, IgG₂, IgG₃, and IgG₄ constant domains in Béranger, et al., Ibid.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following.
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk J. Mol. Biol. 196: 901-917 (1987); Al-Lazikani et al., J. Mol. Biol. 273: 927-948 (1997));
AbM numbering scheme is a compromise between the two used by Oxford Molecular's AbM antibody modelling software (see Karu et al., ILAR Journal 37: 132-141 (1995);
Contact numbering scheme is based on an analysis of the available complex crystal structures (See www.bioinf.org.uk: Prof. Andrew C. R. Martin's Group; Abhinandan & Martin, Mol. Immunol. 45:3832-3839 (2008).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509(1997); Lefranc et al., Dev Comp Immunol. 27:55-77 (2003)).

The following general rules disclosed in www.bioinf.org.uk: Prof Andrew C. R. Martin's Group and reproduced in Table 1 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 1
CDRs in antibody and antigen binding fragments thereof

| Loop | Kabat | AbM | Chothia¹ | Contact² | IMGT |
|---|---|---|---|---|---|
| L1 | L24-L34 | L24-L34 | L24-L34 | L30-L36 | L27-L32 |
| L2 | L50-L56 | L50-L56 | L50-L56 | L46-L55 | L50-L52 |
| L3 | L89-L97 | L89-L97 | L89-L97 | L89-L96 | L89-L97 |
| H1 | H31-H35B | H26-H35B | H26-H32..34 | H30-H35B | H26-H35B |
| | (Kabat | | | | |
| | Numbering)³ | | | | |
| H1 | H31-H35 | H26-H35 | H26-H32 | H30-H35 | H26-H33 |
| | (Chothia | | | | |
| | Numbering) | | | | |
| H2 | H50-H65 | H50-H58 | H52-H56 | H47-H58 | H51-H56 |
| H3 | H95-H102 | H95-H102 | H95-H102 | H93-H101 | H93-H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

As used herein, the term “framework” or “FR” residues refers to those variable domain residues other than the hypervariable region residues defined herein as CDR residues. The residue numbering above relates to the Kabat numbering system and does not necessarily correspond in detail to the sequence numbering in the accompanying Sequence Listing. Amino acid residues in antibodies can also be defined using other numbering systems, such as Chothia, enhanced Chothia, IMGT, Kabat/Chothia composite, Honegger (AHo), Contact, or any other conventional antibody numbering scheme.

An “isolated antibody,” as used herein, is intended to refer to an antibody which is substantially free of other antibodies having different antigenic specificities.

As used herein, “isotype” refers to the antibody class (e.g., IgG (including IgG1, IgG2, IgG3, and IgG4), IgM, IgA (including IgA1 and IgA2), IgD, and IgE antibody) that is encoded by the heavy chain constant region genes of the antibody.

An “effector function” refers to the interaction of an antibody Fc region with an Fc receptor or ligand, or a biochemical event that results therefrom. Exemplary “effector functions” include Clq binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor; BCR). Such effector functions generally require the Fc region to be combined with a binding domain (e.g., an antibody variable domain).

An “Fc region,” “Fc domain,” or “Fc” refers to the C-terminal region of the heavy chain of an antibody. Thus, an Fc region comprises the constant region of an antibody excluding the first constant region immunoglobulin domain (e.g., CH1 or CL).

The term “epitope” or “antigenic determinant” refers to a site on an antigen (e.g., human LAP-TGFβ1) to which an immunoglobulin or antibody specifically binds. Epitopes can be formed both from contiguous amino acids (usually a linear epitope) or noncontiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, 15, 16, 17, 18, 19, 20, 21, or 22 amino acids in a unique spatial conformation.

The term “epitope mapping” refers to the process of identifying the molecular determinants on the antigen involved in antibody-antigen recognition. Methods for determining what epitopes are bound by a given antibody are well known in the art and include, for example, immunoblotting and immunoprecipitation assays, wherein overlapping or contiguous peptides from, e.g., LAP-TGFβ1 are tested for reactivity with a given antibody (e.g., anti-LAP antibody); x-ray crystallography; antigen mutational analysis, two-dimensional nuclear magnetic resonance; yeast display; and hydrogen/deuterium exchange-mass spectrometry (HDX-MS) (see, e.g., Epitope Mapping Protocols in Methods in Molecular Biology, Vol. 66, G. E. Morris, Ed. (1996)). See also Champe et al. (1995) J. Biol. Chem. 270:1388-1394.

The term “binds to the same epitope” with reference to two or more antibodies means that the antibodies bind to the same segment or same segments of amino acid residues, as determined by a given method. Techniques for determining whether antibodies bind to the “same epitope on LAP-TGFβ1” with the antibodies described herein include, for example, epitope mapping methods, such as x-ray analyses of crystals of antigen:antibody complexes, which provides atomic resolution of the epitope, and HDX-MS. Other methods monitor the binding of the antibody to antigen fragments thereof (e.g. proteolytic fragments) or to mutated variations of the antigen where loss of binding due to a modification of an amino acid residue within the antigen sequence is often considered an indication of an epitope component, such as alanine scanning mutagenesis (Cunningham & Wells (1985) Science 244:1081), yeast display of mutant target sequence variants, or analysis of chimeras. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known binding competition experiments, e.g., BIACORE® surface plasmon resonance (SPR) analysis. In certain embodiments, an antibody competes with, and inhibits binding of another antibody to a target by at least 50%, 60%, 70%, 80%, 90% or 100%. The level of inhibition or competition may be different depending on which antibody is the “blocking antibody” (i.e., the antibody that when combined with an antigen blocks another immunologic reaction with the antigen). Competition assays can be conducted as described, for example, in Ed Harlow and David Lane, Cold Spring Harb. Protoc. 2006; doi:10.1101/pdb.prot4277 or in Chapter 11 of “Using Antibodies” by Ed Harlow and David Lane, Cold Spring Harbor Laboratory Press, Cold Spring Harbor, N.Y., USA 1999. Competing antibodies bind to the same epitope, an overlapping epitope, or to adjacent epitopes (e.g., as evidenced by steric hindrance). Two antibodies “cross-compete” if antibodies block each other both ways by at least 50%, i.e., regardless of whether one or the other antibody is contacted first with the antigen in the competition experiment.

Competitive binding assays for determining whether two antibodies compete or cross-compete for binding include competition for binding to cells expressing LAP-TGFβ1, e.g., by flow cytometry. Other methods include: SPR (e.g., BIACORE®), solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., SPR using a predetermined antigen as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Any K_{D} greater than about 10⁻⁴ M is generally considered to indicate nonspecific binding.

The term “k_{assoc}” or “kₐ”, as used herein, refers to the association rate of a particular antibody-antigen interaction, whereas the term “k_{dis}” or “k_{d},” as used herein, is intended to refer to the dissociation rate of a particular antibody-antigen interaction. The term “K_{D}”, as used herein, is intended to refer to the dissociation constant, which is obtained from the ratio of k_{d} to kₐ (i.e., k_{d}/kₐ) and is expressed as a molar concentration (M). K_{D} values for antibodies or antigen binding fragments thereof can be determined using methods well established in the art. A preferred method for determining the K_{D} of an antibody or antigen binding fragment thereof is by using SPR, preferably using a biosensor system such as a Biacore® system or flow cytometry and Scatchard analysis, or bio-layer interferometry.

The term “EC50” in the context of an in vitro or in vivo assay using an antibody or antigen binding fragment thereof refers to the concentration of an antibody or antigen binding fragment thereof that induces a response that is 50% of the maximal response, i.e., halfway between the maximal response and the baseline.

The term “cross-reacts,” as used herein, refers to the ability of an antibody or antigen binding fragment thereof described herein to bind to LAP-TGFβ1 from a different species. For example, an antibody or antigen binding fragment thereof described herein that binds human LAP-TGFβ1 may also bind another species of LAP-TGFβ1 (e.g., murine LAP-TGFβ1, rat LAP-TGFβ1, or cynomolgus monkey LAP-TGFβ1). Cross-reactivity may be measured by detecting a specific reactivity with purified antigen in binding assays (e.g., SPR, ELISA, bio-layer interferometry) or binding to, or otherwise functionally interacting with, cells physiologically expressing LAP-TGFβ1 (e.g., HT1080 cells overexpressing LAP-TGFβ1). Methods for determining cross-reactivity include standard binding assays as described herein, for example, by bio-layer interferometry or flow cytometric techniques.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid molecule,” as used herein in reference to nucleic acids encoding antibodies or antibody binding fragments thereof (e.g., VH, VL, CDR3), is intended to refer to a nucleic acid molecule in which the nucleotide sequences are essentially free of other genomic nucleotide sequences, e.g., those encoding antibodies or antibody binding fragments thereof that bind antigens other than LAP, which other sequences may naturally flank the nucleic acid in human genomic DNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

Also provided are “conservative sequence modifications” of the sequences set forth herein, i.e., amino acid sequence modifications which do not abrogate the binding of the antibody or antigen binding fragment thereof encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into a sequence in a table herein (e.g., Tables 4, 6, 8, 11-43, and 45) by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-LAP antibody is preferably replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). Alternatively, in another embodiment, mutations can be introduced randomly along all or part of an anti-LAP antibody coding sequence or anti-LAP antigen binding fragment thereof coding sequence, such as by saturation mutagenesis, and the resulting modified anti-LAP antibodies can be screened for binding activity.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 80% to 85%, 85% to 90% or 90% to 95%, and more preferably at least about 98% to 99.5% of the nucleotides. Alternatively, substantial homology exists when the segments will hybridize under selective hybridization conditions, to the complement of the strand. For polypeptides, the term “substantial homology” indicates that two polypeptides, or designated sequences thereof, when optimally aligned and compared, are identical, with appropriate amino acid insertions or deletions, in at least about 80% of the amino acids, usually at least about 80% to 85%, 85% to 90%, 90% to 95%, and more preferably at least about 98% to 99.5% of the amino acids.

The percent identity between two sequences is a function of the number of identical positions shared by the sequences (i.e., % homology=#of identical positions/total #of positions ×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or two amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

The term “recombinant host cell” (or simply “host cell”), as used herein, is intended to refer to a cell that comprises a nucleic acid that is not naturally present in the cell, and may be a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “inhibition” as used herein, refers to any statistically significant decrease in biological activity, including partial and full blocking of the activity. For example, “inhibition” can refer to a statistically significant decrease of about 10%, 20%, 30%, 40%, 50%, 60%, 70%, 80%, 90%, 95%, 96%, 97%, 98%, 99%, or 100% in biological activity (e.g., TGFβ1).

As used herein, “TGFβ1 activation” refers to the release of the mature cytokine TGFβ1 from the latent complex made up of LAP and TGFβ1. There are many mechanisms known to induce TGFβ1 activation (see Robertson I B, Rifkin D B. Unchaining the beast; insights from structural and evolutionary studies on TGFβ1 secretion, sequestration, and activation. Cytokine Growth Factor Rev. 2013 August; 24(4):355-72). The mature cytokine can be detected using a specific ELISA or similar detection methodology or through the use of a reporter cell line that expresses a TGFβ receptor.

For example, as used herein, the term “inhibits TGFβ1 activation” includes any measurable decrease in TGFβ1 activation, e.g., an inhibition of TGFβ1 activation by at least about 10%, for example, at least about 20%, at least about 30%, at least about 40%, at least about 50%, at least about 60%, at least about 70%, at least about 80%, at least about 90%, at least about 99%, or about 100%, relative to a control (e.g., a control antibody). The inhibition may be specific to a single mechanism of TGFβ1 activation or may be generalizable to all mechanisms of TGFβ1 activation. As used herein, the term “inhibits TGFβ1 activation” includes inhibition of at least one activation mechanism.

The terms “treat,” “treating,” and “treatment,” as used herein, refer to therapeutic or preventative measures described herein. The methods of “treatment” employ administration to a subject with a tumor or cancer or a subject who is predisposed to having such a disease or disorder, an anti-LAP antibody (e.g., anti-human LAP antibody) or antigen binding fragment thereof described herein, in order to prevent, cure, delay, reduce the severity of, or ameliorate one or more symptoms of the disease or disorder or recurring disease or disorder, or in order to prolong the survival of a subject beyond that expected in the absence of such treatment.

“Immunotherapy” refers to the treatment of a subject afflicted with, or at risk of contracting or suffering a recurrence of, a disease by a method comprising inducing, enhancing, suppressing or otherwise modifying an immune response.

“Immunostimulating therapy” or “immunostimulatory therapy” refers to a therapy that results in increasing (inducing or enhancing) an immune response in a subject for, e.g., treating cancer.

As used herein, “immune cell” refers to the subset of blood cells known as white blood cells, which include mononuclear cells such as lymphocytes, monocytes, macrophages, and granulocytes.

As used herein, “immunosuppressive cell” refers to a cell that contributes to or promotes an immunosuppressive tumor microenvironment. The presence of a population of immunosuppressive cells in a tumor microenvironment increases the tumor's resistance to an immune response, resulting in tumor protection, tumor escape, and/or tumor metastasis. Unless countered in some manner, these immunosuppressive cells can decrease the efficacy of immune-mediated anti-cancer treatments. Exemplary immunosuppressive cells include cancer-associated fibroblasts, myeloid-derived suppressor cells, regulatory T cells (Tregs), tumor cells expressing LAP, and immunosuppressive macrophages. These cell types can be identified by one skilled in the art using, e.g., flow cytometry to identify markers of Tregs (e.g., CD4, FoxP3, CD127, and CD25), macrophages (e.g., CSF-IR, CD203, CD206, CD163, IL-10, and TGFβ), cancer associated fibroblasts (e.g., alpha smooth muscle actin, fibroblast activation protein, tenascin-C, periostin, NG2, vimentin, desmin, PDGFR alpha and beta, FSP-1, ASPN, and STC1), and myeloid-derived suppressor cells (e.g., CD11b, CD33, CD14, or CD15, and low levels of HLA DR). It is understood that immunosuppressive cells may also be important in suppressing the immune system in other disease states.

As used herein, “suppressive T cells” refer to T cells that contribute to or promote an immunosuppressive microenvironment. Exemplary suppressive T cells include CD4+ regulatory T cells and CD8+ regulatory T cells. Such cells can be identified by one skilled in the art using, e.g., flow cytometry to identify markers such as FoxP3, LAP or Helios.

As used herein, “regulatory T cells” or “Tregs” refer to immunosuppressive cells that generally suppress or downregulate induction and proliferation of effector T cells. Tregs may express the biomarkers CD4, FOXP3, and CD25 and are thought to be derived from the same lineage as naïve CD4 cells.

“T effector” (“Teff”) cells refers to T cells (e.g., CD4+ and CD8+T cells) with cytolytic activities as well as T helper (Th) cells, which secrete inflammatory cytokines and activate and direct other immune cells, but does not include regulatory T cells (Treg cells).

As used herein, “administering” refers to the physical introduction of a molecule (e.g., an antibody or antigen binding fragment thereof that binds LAP as described herein) or of a composition comprising a therapeutic agent (e.g., an anti-LAP antibody or antigen binding fragment thereof as described herein) to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody or antigen binding fragment thereof as described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, “cancer” refers to a broad group of diseases characterized by the uncontrolled growth of abnormal cells in the body. Unregulated cell division may result in the formation of malignant tumors or cells that invade neighboring tissues and may metastasize to distant parts of the body through the lymphatic system or bloodstream.

As used herein, “autoimmune disease” describes a disease state or syndrome whereby a subject's body produces a dysfunctional immune response against the subject's own body components, with adverse effects.

As used herein, “fibrosis” refers to disorders or disease states that are caused by or accompanied by the abnormal deposition of extracellular matrix (i.e., not formation of fibrous tissue in normal organ and tissue). Fibrosis is characterized by excessive accumulation of extracellular matrix in the affected tissue that often results in destruction of its normal architecture and causes significant organ dysfunction. Although fibrotic conditions in various organs have diverse etiologies, fibrosis typically results from chronic persistent inflammation induced by a variety of stimuli, such as chronic infections, ischemia, allergic and autoimmune reactions, chemical insults or radiation injury (from Biernacka, 2011 Growth Factors. 2011 Oct.; 29(5):196-202. doi: 10.3109/08977194.2011.595714. Epub 2011 Jul. 11). Fibrosis may affect the heart, liver, kidney, lung and skin and is also a central feature in many cancers. As used herein, “cell therapy” refers to a method of treatment involving the administration of live cells (e.g., CAR T cells, and NK cells).

The terms “treat,” “treating,” and “treatment,” as used herein, refer to any type of intervention or process performed on, or administering an active agent (e.g., an anti-LAP antibody or antigen binding fragment thereof) to, the subject with the objective of reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

As used herein, “adjunctive” or “combined” administration (co-administration) includes simultaneous administration of the agents and/or compounds in the same or different dosage form, or separate administration of the compounds (e.g., sequential administration). For example, at least one agent comprises an anti-LAP antibody or antigen binding fragment thereof. Thus, a first antibody or antigen binding fragment thereof, e.g., an anti-LAP antibody or antigen binding fragment thereof, and a second, third, or more antibodies or antigen binding fragments thereof can be simultaneously administered in a single formulation. Alternatively, the first and second (or more) antibodies or antigen binding fragments thereof can be formulated for separate administration and are administered concurrently or sequentially.

“Combination” therapy, as used herein, means administration of two or more therapeutic agents in a coordinated fashion, and includes, but is not limited to, concurrent dosing. Specifically, combination therapy encompasses both co-administration (e.g. administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. (See, e.g., Kohrt et al. (2011) Blood 117:2423). For example, the anti-LAP antibody can be administered first followed by (e.g., immediately followed by) the administration of a second antibody (e.g., an anti-PD-1 antibody) or antigen binding fragment thereof, or vice versa. In one embodiment, the anti-LAP antibody or antigen binding fragment thereof is administered prior to administration of the second antibody or antigen binding fragment thereof. In another embodiment, the anti-LAP antibody or antigen binding fragment thereof is administered, for example, a few minutes (e.g., within about 30 minutes) or at least one hour of the second antibody or antigen binding fragment thereof. Such concurrent or sequential administration preferably results in both antibodies or antigen binding fragments thereof being simultaneously present in treated patients.

The term “effective dose” or “effective dosage” is defined as an amount sufficient to achieve or at least partially achieve a desired effect. A “therapeutically effective amount” or “therapeutically effective dosage” of a drug (e.g., anti-LAP antibody or antigen binding fragment thereof) is any amount of the drug or therapeutic agent that, when used alone or in combination with another therapeutic agent, promotes disease regression evidenced by a decrease in severity of disease symptoms, an increase or cessation in frequency and duration of disease symptom-free periods, or a prevention of impairment or disability due to the disease affliction. A therapeutically effective amount or dosage of a drug or therapeutic agent includes a “prophylactically effective amount” or a “prophylactically effective dosage”, which is any amount of the drug or therapeutic agent that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

The administration of effective amounts of the anti-LAP antibody or antigen binding fragment thereof alone, or anti-LAP antibody or antigen binding fragment thereof combined with another compound or agent (e.g., an immune checkpoint blocker such as an anti-PD-1 antibody), according to any of the methods provided herein, can result in at least one therapeutic effect, including, for example, reduced tumor growth or size, reduced number of indicia of cancer (e.g., metastatic lesions) appearing over time, complete remission, partial remission, or stable disease. For example, the methods of treatment produce a comparable clinical benefit rate (CBR=complete remission (CR)+ partial remission (PR)+stable disease (SD) lasting≥6 months) better than that achieved without administration of the anti-LAP antibody or antigen binding fragment thereof, or than that achieved with administration of any one of the combined antibodies, e.g., the improvement of clinical benefit rate is about 20%, 30%, 40%, 50%, 60%, 70%, 80% or more.

By way of example, for the treatment of tumors, a therapeutically effective amount or dosage of the drug or therapeutic agent (e.g., anti-LAP antibody or antigen binding fragment thereof) inhibits tumor cell growth by at least about 20%, by at least about 30% by at least about 40%, by at least about 50%, by at least about 60%, by at least above 70%, by at least about 80%, or by at least about 90% relative to untreated subjects. In some embodiments, a therapeutically effective amount or dosage of the drug or therapeutic agent completely inhibits cell growth or tumor growth, i.e., inhibits cell growth or tumor growth by 100%. The ability of a compound or therapeutic agent, including an antibody, to inhibit tumor growth can be evaluated using the assays described herein. Alternatively, this property of a composition comprising the compound or therapeutic agent can be evaluated by examining the ability of the composition to inhibit cell growth; such inhibition can be measured in vitro by assays known to the skilled practitioner.

The term “patient” includes human and other mammalian subjects that receive either prophylactic or therapeutic treatment.

As used herein, the term “subject” includes any human or non-human animal. For example, the methods and compositions described herein can be used to treat a subject having cancer. The term “non-human animal” includes all vertebrates, e.g., mammals and non-mammals, such as non-human primates, sheep, cats, dogs, cows, chickens, amphibians, reptiles, etc.

The term “sample” refers to tissue, bodily fluid, or a cell (or a fraction of any of the foregoing) taken from a patient or a subject. Normally, the tissue or cell will be removed from the patient, but in vivo diagnosis is also contemplated. In the case of a solid tumor, a tissue sample can be taken from a surgically removed tumor and prepared for testing by conventional techniques. In the case of lymphomas and leukemias, lymphocytes, leukemic cells, or lymph tissues can be obtained (e.g., leukemic cells from blood) and appropriately prepared. Other samples, including urine, tears, serum, plasma, cerebrospinal fluid, feces, sputum, cell extracts etc. can also be useful for particular cancers.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising,” “consisting essentially of,” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

As used herein, the singular forms “a,” “an,” and “the” include plural referents unless the context clearly dictates otherwise. The use of “or” or “and” means “and/or” unless stated otherwise. Furthermore, use of the term “including” as well as other forms, such as “include,” “includes,” and “included,” is not limiting.

The term “about” as used herein when referring to a measurable value such as an amount, a temporal duration and the like, encompasses variations of up to ±10% from the specified value. Unless otherwise indicated, all numbers expressing quantities of ingredients, properties such as molecular weight, reaction conditions, etc., used herein are to be understood as being modified by the term “about”.

As used herein, “and/or” is to be taken as specific disclosure of each of the two specified features or components with or without the other. Thus, the term “and/or” as used in a phrase such as “A and/or B” includes “A and B,” “A or B,” “A” alone, and “B” alone. Likewise, the term “and/or” as used in a phrase such as “A, B, and/or C” encompasses each of the following: A, B, and C; A, B, or C; A or C; A or B; B or C; A and C; A and B; B and C; A alone; B alone; and C alone.

As used herein, the terms “ug” and “uM” are used interchangeably with “μg” and “μM,” respectively.

Various aspects described herein are described in further detail in the following subsections.

The location of the LAP-TGFβ1 complex is of critical biological and clinical importance because, once the mature TGFβ1 cytokine, which has a short half-life in solution, is released, it acts locally, either in an autocrine or near paracrine fashion. Therefore, the anchor proteins are a principal mechanism whereby latent TGFβ1 is staged in a specific location, awaiting the release of the potent mature cytokine to act on the local tissue.

LAP-TGFβ1 has different functions when expressed in different locations. For example, LAP-TGFβ1 anchored by LTBPs in the extracellular matrix is of primary importance for tissue homeostasis. In this regard, Xu et al. (Bone Research 2018; 6:2) noted that “the TGF-β complex is more like a molecular sensor that responds instantly to ECM perturbations through the release of an active ligand that exerts physiological effects at a cellular level, thus ensuring normal tissue homeostasis.”

Alterations in LAP-TGFβ1 incorporation into the extracellular matrix are known to result in human disease. For example, deletion of LTBP-3 in both mice and humans results in similar defects in both bone and dental formation. LTBP-3 defects are also associated with the aortic dilation seen in Marfan syndrome (Rifkin et al., Matrix Biol 2018; 71-72:90-99). These effects are believed to be due to aberrant direct effects of TGFβ1 in the local extracellular matrix (Xu et al, Bone Research 2018; 6:2).

In contrast to anchor proteins that localize LAP-TGFβ1 to the extracellular matrix, LAP-TGFβ1 anchored by GARP is of primary importance for the immunosuppressive function of regulatory T cells (Edwards et al, Eur J Immunol 2016; 46:1480-9) and of suppressive B cell subpopulations (Wallace et al, JCI Insight 2018; 3:e99863). Some tumors have also been shown to express GARP, allowing them to locally express TGFβ and directly suppress the immune system in the tumor microenvironment and support their own growth (Metelli et al, Journal of Hematology & Oncology 2018; 11:24).

LAP-TGFβ1 anchored to myeloid cells is of primary importance for the immunosuppressive function of MDSCs (Zhang H et al., Frontiers in Immunology 2017; 8:1-15) and of M2 macrophages (Zhang et al., Oncotarget 2017; 8:99801-15). According to a recent study, myeloid cells have been shown to use the anchor protein LRRC33 to anchor latent TGFβ to the cell surface (Qin et al., Cell 2018; 174:1-16).

### I. Anti-LAP Antibodies

In one aspect, provided herein is an isolated anti-LAP antibody (i.e., an antibody that binds LAP) or antigen binding fragment thereof.

In one aspect, provided herein is an isolated anti-LAP antibody (e.g., recombinant humanized, chimeric, or human antibody) or antigen binding fragment thereof which comprises an amino acid sequence described herein:

Functional features of the anti-LAP antibodies or antigen binding fragment thereof provided herein are described below in more detail.

In some embodiments, the anti-LAP antibody or antigen binding fragment thereof described herein binds to LAP-TGFβ1 (e.g., human LAP-TGFβ1) in the absence of an anchor protein. For example, the anti-LAP antibody or antigen binding fragment thereof described herein binds to recombinant human LAP-TGFβ1 in an assay that does not include an anchor protein. [start here]

In some embodiments, the anti-LAP antibody or antigen binding fragment thereof described herein binds to LAP-TGFβ1 (e.g., soluble LAP-TGFβ1) with a K_{D} of 100 nM or less, such as 90 nM or less, 80 nM or less, 70 nM or less, 60 nM or less, 50 nM or less, such as 40 nM or less, 30 nM or less, 20 nM or less, 10 nM or less, 5 nM or less, 3 nM or less, 1 nM or less, 0.9 nM or less, 0.8 nM or less, 0.7 nM or less, 0.6 nM or less, 0.5 nM or less, 0.4 nM or less, 0.3 nM or less, 0.2 nM or less, 0.1 nM or less, 10 nM to 0.1 nM, 5 nM to 0.1 nM, 3 nM to 0.1 nM, 1 nM to 0.1 nM, 0.8 nM to 0.1 nM, 0.5 nM to 0.1 nM, 10 nM to 0.5 nM, 10 nM to 0.8 nM, 10 nM to 1 nM, 1 nM to 0.5 nM, or 1 nM to 0.8 nM, as assessed by, e.g., bio-layer interferometry, or as determined by Octet or BIACore. In some embodiments, the anti-LAP antibody or antigen binding fragment thereof described herein binds to LAP-TGFβ1 (e.g., human, cyno, and rat) with a K_{D} in an Example herein. In various embodiments, the anti-LAP antibody or antigen binding fragment thereof described herein binds to human LAP-TGFβ1, rat LAP-TGFβ1, cyno LAP-TGFβ1, and/or murine LAP-TGFβ1.

In some embodiments, the anti-LAP antibody or antigen binding fragment thereof described herein described herein binds to LAP-TGFβ1 complexed with an anchor protein on immunosuppressive cells, but does not bind to the anchor protein. In some embodiments, the anchor protein is GARP or LRRC33.

In some embodiments, the anti-LAP antibody or antigen binding fragment thereof described herein described herein selectively inhibits TGFβ1 activation on immunosuppressive cells without inhibiting TGFβ1 activation on extracellular matrix.

In some embodiments, the anti-LAP antibody or antigen binding fragment thereof described herein does not bind to LAP complexed with LTBP1, LTBP3, and/or LTBP4.

In some embodiments, the anti-LAP antibody or antigen binding fragment thereof described herein does not bind to LAP-TGFβ2 (e.g., human LAP-TGFβ2) and LAP-TGFβ3 (e.g., human LAP-TGFβ3), as assessed by, e.g., flow cytometry using cells that overexpress TGFβ2 or TGFβ3, or bio-layer interferometry with recombinant LAP-TGFβ2 or LAP-TGFβ3. For example, in some embodiments, the anti-LAP antibody or antigen binding fragment thereof described herein binds to LAP-TGFβ2 or LAP-TGFβ3 with a signal or affinity that is not significantly above the signal seen with a control antibody (e.g., isotype control) or the signal seen in the absence of anti-LAP antibody.

In some embodiments, the anti-LAP antibody or antigen binding fragment thereof described herein inhibits TGFβ1 activation, as assessed by, e.g., ELISA detection of free TGFβ1 in a culture of P3U1 cells overexpressing LAP-TGFβ1. In some embodiments, the anti-LAP antibody or antigen binding fragment thereof described herein inhibits (or is determined to inhibit) TGFβ1 activation by about 50% or more, e.g., by about 60% or more, by about 70% or more, by about 80% or more, or by about 90% or more, as assessed by ELISA, e.g., ELISA detection of free TGFβ1 in a culture of P3U1 cells overexpressing LAP-TGFβ1.

In some embodiments, the anti-LAP antibody or antigen binding fragment thereof described herein binds to mouse and human LAP-TGFβ1, as assessed by, e.g., flow cytometry of activated immune cell populations.

In some embodiments, the anti-LAP antibody or antigen binding fragment thereof described herein does not bind to free TGFβ1 (i.e., TGFβ1 without LAP), as assessed by, e.g., ELISA. In some embodiments, the anti-LAP antibody or antigen binding fragment thereof described herein does not bind to empty LAP (i.e., LAP that is not complexed with TGFβ1), as assessed by, e.g., bio-layer interferometry. For example, in some embodiments, the anti-LAP antibody or antigen binding fragment thereof described herein binds to free TGFβ1 or empty with a signal or affinity that is not significantly above the signal seen with a control antibody (e.g., isotype control) or the signal seen in the absence of anti-LAP antibody.

In some embodiments, the anti-LAP antibody or antigen binding fragment thereof described herein binds to human LAP-TGFβ1 comprising K27C and Y75C mutations (SEQ ID NO: 12. In another embodiment, the anti-LAP antibody or antigen binding fragment thereof described herein does not bind to (or are determined not to bind to) human LAP-TGFβ1 comprising a Y74T mutation (SEQ ID NO: 13). In another embodiment, the anti-LAP antibody or antigen binding fragment thereof described herein binds to (or is determined to bind to) human LAP-TGFβ1 comprising K27C and Y75C mutations, but not to LAP-TGFβ1 comprising a Y74T mutation.

In some embodiments, the anti-LAP antibodies bind to all or a portion of residues 82-130 of human LAP-TGFβ1 (SEQ ID NO: 1).

In some embodiments, the anti-LAP antibodies bind within residues 82-130 of human LAP-TGFβ1 (SEQ ID NO: 1). In some embodiments, the anti-LAP antibody or antigen binding fragment thereof binds to one or more regions on human LAP-TGFβ1 (SEQ ID NO: 1) comprising or consisting of amino acids 31-40, 274-280, and 340-343. In some embodiments, the anti-LAP antibody or antigen binding fragment thereof binds to amino acids 31-40, 274-280, and 340-343 of human LAP-TGFβ1 (SEQ ID NO: 1). In some embodiments, the epitope is determined by cryo-EM.

In some embodiments, the anti-LAP antibody or antigen binding fragment thereof binds to one or more regions on human a LAP-TGFβ1 (SEQ ID NO: 1) comprising or consisting of amino acids 31-38, 278-281, and 342-344. In some embodiments, the anti-LAP antibodies bind to amino acids 31-38, 278-281, and 342-344 of human LAP-TGFβ1 (SEQ ID NO: 1). In some embodiments, the epitope is determined by cryo-EM. In some embodiments, the anti-LAP antibody or antigen binding fragment thereof binds to one or more regions on human a LAP-TGFβ1 (SEQ ID NO: 1) comprising or consisting of amino acids 35-43, 272-275, 280-283, and 340 (SEQ ID NO: 1). In some embodiments, the anti-LAP antibody or antigen binding fragment thereof binds to amino acids 35-43, 272-275, 280-283, and 340 of human LAP-TGFβ1 (SEQ ID NO: 1). In some embodiments, the epitope is determined by cryo-EM. In various embodiments, the epitope is described in a Table herein, for example, Table 58.

As discussed above, the anti-LAP antibody or antigen binding fragment thereof described herein binds to LAP-TGFβ1 on cells, such as immune cells, e.g., immunosuppressive cells. Immunosuppressive cells include, but are not limited to, suppressive T cells (e.g., regulatory T cells, activated T cells, suppressive CD8+ T cells), M1 macrophages, M2 macrophages, dendritic cells, regulatory B cells, granulocytic MDSCs, and/or monocytic MDSCs, as assessed, e.g., by flow cytometry. In some embodiments, the anti-LAP antibody or antigen binding fragment thereof described herein binds to cells other than immune cells, such as tumor cells, fibroblasts (including cancer associated fibroblasts), mesenchymal stromal cells, mesenchymal stem cells, hemopoietic stem cells, non-myelinating Schwann cells, myofibroblasts, endothelial cells, platelets, megakaryocytes, pericytes, and/or hepatic stellate cells. In some embodiments, the anti-LAP antibody or antigen binding fragment thereof described herein binds to LAP-TGFβ1 on both immune cells (e.g., immunosuppressive cells) and non-immune cells.

In some embodiments, the anti-LAP antibody or antigen binding fragment thereof described herein binds to LAP-TGFβ1 on GARP-positive cells (e.g., GARP-positive immunosuppressive cells). In some embodiments, the anti-LAP antibody or antigen binding fragment thereof described herein binds to (or are determined to bind to) LAP-TGFβ1 on GARP-negative cells (e.g., GARP-negative immunosuppressive cells). In some embodiments, the anti-LAP antibody or antigen binding fragment thereof described herein binds to LAP-TGFβ1 on both GARP-positive and GARP-negative cells, as assessed, e.g., by flow cytometry.

In some embodiments, the anti-LAP antibody or antigen binding fragment thereof described herein reduces the endogenous expression of CD73. In some embodiments, the anti-LAP antibody or antigen binding fragment thereof described herein inhibits the increase of CD73 expression caused by a treatment, e.g., radiation. CD73 expression can be determined using standard methods known in the art.

In some embodiments, the anti-LAP antibody or antigen binding fragment thereof described herein binds to LAP-TGFβ1 expressed on cells (e.g., human or mouse LAP-TGFβ1 expressed on, e.g., P3U1 cells) with an EC₅₀ of 1000 nanogram per milliliter (ng/mL) or less, 500 ng/mL or less, 200 ng/mL or less, 150 ng/mL or less, 100 ng/mL or less, 50 ng/mL or less, 25 ng/mL or less, 10 ng/mL or less, 5 ng/mL or less, 2 ng/mL or less, 1 ng/mL to 200 ng/mL, 1 ng/mL to 150 ng/mL, 1 ng/mL to 100 ng/mL, 1 ng/mL to 50 ng/mL, 1 ng/mL to 25 ng/mL, 1 ng/mL to 10 ng/mL, or 1 ng/mL to 5 ng/mL, as measured by flow cytometry.

The binding of the anti-LAP antibody or antigen binding fragment thereof to LAP-TGFβ1 may also be defined using quantitative immunofluorescence by flow cytometry, which allows the number of antibody molecules bound per cell to be quantified. Accordingly, in some embodiments, the number of anti-LAP antibodies bound to a cell that also expresses GARP may be equal to the number of anti-GARP antibodies bound to that cell, or may be at least 80%, at least 50%, at least 20%, at least 10%, at least 5%, at least 1%, or at least 0.1% of the number of anti-GARP antibodies bound to that cell. In some embodiments, the number of LAP-TGFβ1 molecules expressed per cell may be quantified using quantitative immunofluorescence using an anti-LAP antibody of a group that detects the majority of LAP molecules; examples of such antibodies include 2F8, 2C9, 16B4 and the anti-LAP monoclonal antibody #27232 (R&D Systems). In some embodiments, the number of anti-LAP antibodies bound to the cell may be equal to the number of LAP molecules on the cell, or may be at least 80%, at least 50%, at least 20%, at least 10%, at least 5%, at least 1% or at least 0.1% of the number of LAP molecules expressed on that cell.

In some embodiments, the anti-LAP antibody or antigen binding fragment thereof described herein inhibits TGFβ1 activation by, for example, 10% or more, for example, 20% or more, 30% or more, 40% or more, 50% or more, 60% or more, 70% or more, 80% or more, 90% or more, or 95% or more, relative to a control (e.g., a control antibody), as measured by ELISA.

Preferably, the anti-LAP antibody or antigen binding fragment thereof described herein binds to soluble LAP-TGFβ1 with high affinity, for example, with a K_{D} of 10⁻⁷ M or less, 10⁻⁸ M or less, 10⁻⁹ M or less, 10⁻¹⁰ M or less, 10⁻¹¹ M or less, 10⁻¹² M or less, 10⁻¹² M to 10⁻⁷ M, 10⁻¹¹ M to 10⁻⁷ M, 10⁻¹⁰ M to 10⁻⁷ M, or 10⁻⁹ M to 10⁻⁷ M, as measured by bio-layer interferometry.

In some embodiments, the anti-LAP antibody or antigen binding fragment thereof described herein does not bind to LAP-TGFβ1 in the extracellular matrix. For example, the anti-LAP antibody or antigen binding fragment thereof described herein do not bind to LAP-TGFβ1 in the extracellular matrix, as assessed by ELISA, wherein the O.D. signal for the antibody or antigen binding fragment thereof binding is not significantly above the signal seen in the absence of the anti-LAP antibody or antigen binding fragment thereof described herein or the signal seen with a control antibody (e.g., isotype control).

In some embodiments, the anti-LAP antibody or antigen binding fragment thereof described herein do not inhibit TGFβ activation in the ECM, as assessed by, e.g., ELISA detection of free TGFβ1 in an assay combining a source of LAP-TGFβ1 in the ECM with MMP-2, MMP-9, thrombospondin or cells expressing αVβ6 or αVβ8 integrins.

In some embodiments, the anti-LAP antibody or antigen binding fragment thereof described herein binds to LAP-TGFβ1 on platelets. For example, in some embodiments, at least 5%, at least 10%, at least 20% or at least 50% of platelets can be detected by binding of the anti-LAP antibody (e.g. display a signal above that seen with an isotype control antibody) by flow cytometry. In some embodiments, the anti-LAP antibody or antigen binding fragment thereof described herein binds to platelets but do not cause platelet aggregation or platelet degranulation.

In some embodiments, the anti-LAP antibody or antigen binding fragment thereof described herein binds to immune cells, e.g., suppressive T cells (e.g., regulatory T cells), M2 macrophages, monocytic MDSCs, CD11b-positive cells, and/or dendritic cells. For example, in some embodiments, at least 0.5%, at least 1%, at least 2%, at least 5%, at least 7%, at least 10%, at least 20%, or at least 50% of these cell types can be detected by binding of the anti-LAP antibody (e.g. display a signal above that seen with an isotype control antibody) by flow cytometry. In some embodiments, the anti-LAP antibody or antigen binding fragment thereof described herein is considered to bind to these cell types if they bind ≥2 standard deviations above isotype control.

In some embodiments, the anti-LAP antibody or antigen binding fragment thereof described herein binds to GARP-negative leukocytes. For example, in some embodiments, at least 0.5%, at least 1%, at least 2%, at least 5%, at least 7%, at least 10%, at least 20% or at least 50% of GARP-negative leukocytes can be detected by binding of the anti-LAP antibody (e.g. display a signal above that seen with an isotype control antibody) by flow cytometry.

An antibody or antigen binding fragment thereof that exhibits one or more of the functional properties described above (e.g., biochemical, immunochemical, cellular, physiological or other biological activities), as determined using methods known to the art and described herein, will be understood to relate to a statistically significant difference in the particular activity relative to that seen in the absence of the antibody (e.g., or when a control antibody of irrelevant specificity is present). Preferably, the anti-LAP antibody-induced increases in a measured parameter effects a statistically significant increase by at least 10% of the measured parameter, more preferably by at least 20%, 30%, 40%, 50%, 60%, 70%, 80%, 90%, 95%, or 100% (i.e., 2-fold), 3-fold, 5-fold or 10-fold. Conversely, anti-LAP antibody-induced decreases in a measured parameter (e.g., TGFβ1 activation) effects a statistically significant decrease by at least 10%, 20%, 30%, 40%, 50%, 60%, 70%, 80%, 90%, 95%, 97%, 98%, 99%, or 100%.

Also provided herein are anti-LAP antibodies that bind to the same epitope on human LAP-TGFβ1 as any of the anti-LAP antibodies described herein. These antibodies have the ability to cross-compete for binding to human LAP-TGFβ1 with any of the anti-LAP antibodies described herein.

Antibodies disclosed herein include all known forms of antibodies and other protein scaffolds with antibody-like properties. For example, the antibody can be a human antibody, a humanized antibody, a bispecific antibody, an immunoconjugate, a chimeric antibody, or a protein scaffold with antibody-like properties, such as fibronectin or ankyrin repeats.

In some embodiments, the antibody is a bispecific antibody comprising a first and second binding region, wherein the first binding region comprises the binding specificity (e.g., antigen binding region) of an anti-LAP antibody described herein, and a second binding region that does not bind to LAP. In some embodiments, the second binding region binds to a protein that is not expressed on platelets.

The antibody also can be a Fab, F(ab′)₂, scFv, AFFIBODY, avimer, nanobody, single chain antibody, or a domain antibody. The antibody also can have any isotype, including any of the following isotypes: IgG1, IgG2, IgG3, IgG4, IgM, IgA1, IgA2, IgAsec, IgD, and IgE. Full-length antibodies can be prepared from VH and VL sequences using standard recombinant DNA techniques and nucleic acid encoding the desired constant region sequences to be operatively linked to the variable region sequences.

In certain embodiments, the antibodies described herein may have effector function or may have reduced or no effector function. In certain embodiments, anti-LAP antibodies comprise an effector-less or mostly effector-less Fc, e.g., IgG2 or IgG4. Generally, variable regions described herein may be linked to an Fc comprising one or more modification, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, to alter one or more functional properties of the antibody. Each of these embodiments is described in further detail below. The numbering of residues in the Fc region is that of the EU index of Kabat.

In some embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, modifications can be made in the Fc region in order to generate an Fc variant that (a) has increased or decreased antibody-dependent cell-mediated cytotoxicity (ADCC), (b) increased or decreased complement mediated cytotoxicity (CDC), (c) has increased or decreased affinity for C1q and/or (d) has increased or decreased affinity for a Fc receptor relative to the parent Fc. Such Fc region variants will generally comprise at least one amino acid modification in the Fe region. Combining amino acid modifications is thought to be particularly desirable. For example, the variant Fc region may include two, three, four, five, etc. substitutions therein, e.g. of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fe domains can still form a dimeric Fe domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli such as proline iminopeptidase. In other embodiments, one or more glycosylation sites within the Fe domain may be removed. Residues that are typically glycosylated (e.g., asparagine) may confer cytolytic response. Such residues may be deleted or substituted with unglycosylated residues (e.g., alanine). In other embodiments, sites involved in interaction with complement, such as the C1q binding site, may be removed from the Fc region. For example, one may delete or substitute the EKK sequence of human IgG1. In certain embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgG1. Specific examples of variant Fe domains are disclosed for example, in PCT Publication numbers WO 97/34631 and WO 96/32478.

In one embodiment, the hinge region of Fe is modified such that the number of cysteine residues in the hinge region is altered, e.g., increased or decreased. This approach is described further in U.S. Pat. No. 5,677,425 by Bodmer et al. More specifically, one or more amino acid mutations are introduced into the CH2-CH3 domain interface region of the Fc-hinge fragment thereof such that the antibody has impaired Staphylococcyl protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al. In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al. In another example, one or more amino acid residues within amino acid positions 231 and 239 are altered to thereby alter the ability of the antibody to fix complement. This approach is described further in PCT Publication number WO 94/29351 by Bodmer et al.

In yet another example, the Fc region may be modified to increase antibody dependent cellular cytotoxicity (ADCC) and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F/324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Kabat (PCT Patent Publication number WO00/42072).

Other Fc modifications that can be made to Fcs are those for reducing or ablating binding to FcγR and/or complement proteins, thereby reducing or ablating Fc-mediated effector functions such as ADCC, ADCP, and CDC. Exemplary modifications include but are not limited substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcγR and complement interactions include substitutions 297A, 234A, 235A, 237A, 318A, 228P, 236E, 268Q, 309L, 330S, 331 S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691. Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; PCT Patent Publication numbers WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

Fc variants that enhance affinity for an inhibitory receptor FcγRIIb may also be used. Such variants may provide an Fc fusion protein with immunomodulatory activities related to FcγRIIb⁺ cells, including for example B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRllb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F.

In certain embodiments, the antibody is modified to increase its biological half-life. Various approaches are possible. For example, this may be done by increasing the binding affinity of the Fc region for FcRn. For example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 434H, 434F, 434Y, and 434M. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 307Q, 31 1A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 31 1 S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In certain embodiments, hybrid IgG isotypes with particular biological characteristics may be used. For example, an IgG1/IgG3 hybrid variant may be constructed by substituting IgG1 positions in the CH2 and/or CH3 region with the amino acids from IgG3 at positions where the two isotypes differ. Thus, a hybrid variant IgG antibody may be constructed that comprises one or more substitutions, e.g., 274Q, 276K, 300F, 339T, 356E, 358M, 384S, 392N, 397M, 4221, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Thus a hybrid variant IgG antibody may be constructed that comprises one or more substitutions, e.g., one or more of the following amino acid substitutions: 233E, 234L, 235L, −236G (referring to an insertion of a glycine at position 236), and 327A.

Moreover, the binding sites on human IgG1 for FcγR1, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include: S298A/E333A/L334A, S239D/1332E, S239D/1332E/A330L, L235V/F243L/R292P/Y300L/P396L, and M428L/N434S.

When using an IgG4 constant domain, it is usually preferable to include the substitution S228P, which mimics the hinge sequence in IgG1 and thereby stabilizes IgG4 molecules.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Glycosylation of the constant region on N297 may be prevented by mutating the N297 residue to another residue, e.g., N297A, and/or by mutating an adjacent amino acid, e.g., 298 to thereby reduce glycosylation on N297.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication number WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication number WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180).

Another modification of the antibodies described herein is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment thereof. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See for example, European patent number EP 0 154 316 by Nishimura et al. and European patent number EP 0 401 384 by Ishikawa et al.

The affinities and binding properties of an Fc region for its ligand may be determined by a variety of in vitro assay methods (biochemical or immunological based assays) known in the art including, but not limited to, equilibrium methods (e.g., enzyme-linked immunosorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

II. Antibodies which Bind to Same Epitope as or Cross-Compete with Anti-LAP Antibodies

Anti-LAP antibodies which bind to the same or similar epitopes to the antibodies disclosed herein (and thus also cross-compete with the antibodies disclosed herein) may be raised using immunization protocols. The resulting antibodies can be screened for high affinity binding to human LAP-TGFβ1. Selected antibodies can then be studied, e.g., in yeast display assay in which sequence variants of LAP-TGFβ1 are presented on the surface of yeast cells, or by hydrogen-deuterium exchange experiments, to determine the precise epitope bound by the antibody.

Antibodies which bind to the same epitope as the anti-LAP antibodies described herein can also be generated using chimeric constructs, e.g., chicken-human chimeras of LAP-TGFβ1. Since human and chicken sequences can be combined to yield a LAP-TGFβ1 protein that folds correctly, the method can be used to generate immunogens to specific epitopes of interest on LAP-TGFβ1. With this strategy, the majority of the sequence would be taken from chicken LAP-TGFβ1, with small sections of human LAP-TGFβ1 inserted in regions containing the desired epitope. Exemplary epitopes on LAP-TGFβ1 that can be targeted using this strategy include, for example, the lower arm of LAP-TGFβ1, the latency loop of LAP-TGFβ1, or an epitope comprising amino acids 82-130 of human LAP-TGFβ1. This chimeric protein could be used to immunize chickens to yield monoclonal antibodies. Since the chicken LAP-TGFβ1 would be recognized as self, the immune response will be focused on the human sequence. Antibodies generated using this approach can be tested for various functions/properties (e.g., binding to LAP-TGFβ1, inhibiting TGFβ1 activation, binding to ECM, binding to cells such as immunosuppressive cells) using standard methods known in the art, e.g., the methods described herein.

The epitope to which an antibody binds can be determined using art-recognized methods. An anti-LAP antibody is considered to bind to the same epitope as a reference anti-LAP antibody if it, e.g., contacts one or more of the same residues on human LAP-TGFβ1 as the reference antibody; contacts one or more of the same residues within at least one region of human LAP-TGFβ1 as the reference antibody; contacts a majority of residues within at least one region of human LAP-TGFβ1 as the reference antibody; contacts a majority of the same residues within each region of human LAP-TGFβ1 as the reference antibody; contacts a majority of the same residues along the entire length of human LAP-TGFβ1 as the reference antibody; contacts all of the same distinct regions of human LAP-TGFβ1 as the reference antibody; contacts all of the same residues at any one region on human LAP-TGFβ1 as the reference antibody; or contacts all of the same residues at all of the same regions of human LAP-TGFβ1 as the reference antibody.

Techniques for determining antibodies that bind to the “same epitope on human LAP-TGFβ1” with the anti-LAP antibodies described herein include x-ray analyses of crystals of antigen:antibody complexes, which provides atomic resolution of the epitope. Other methods monitor the binding of the antibody to antigen binding fragments thereof or mutated variations of the antigen where loss of binding due to an amino acid modification within the antigen sequence indicates the epitope component. Methods may also rely on the ability of an antibody of interest to affinity isolate specific short peptides (either in native three-dimensional form or in denatured form) from combinatorial phage display peptide libraries or from a protease digest of the target protein. The peptides are then regarded as leads for the definition of the epitope corresponding to the antibody used to screen the peptide library. For epitope mapping, computational algorithms have also been developed that have been shown to map conformational discontinuous epitopes.

The epitope or region comprising the epitope can also be identified by screening for binding to a series of overlapping peptides spanning human LAP-TGFβ1. Alternatively, the method of Jespers et al. (1994) Biotechnology 12:899 may be used to guide the selection of antibodies having the same epitope and therefore similar properties to the anti-LAP antibodies described herein. Using phage display, first, the heavy chain of the anti-LAP antibody is paired with a repertoire of (e.g., human) light chains to select a LAP-binding antibody, and then the new light chain is paired with a repertoire of (e.g., human) heavy chains to select a (e.g., human) LAP-binding antibody having the same epitope or epitope region as an anti-LAP antibody described herein. Alternatively, variants of an antibody described herein can be obtained by mutagenesis of cDNA sequences encoding the heavy and light chains of the antibody.

Alanine scanning mutagenesis, as described by Cunningham & Wells (1989) Science 244: 1081, or some other form of point mutagenesis of amino acid residues in LAP-TGFβ1 may also be used to determine the functional epitope for an anti-LAP antibody.

The epitope or epitope region (an “epitope region” is a region comprising the epitope or overlapping with the epitope) bound by a specific antibody may also be determined by assessing binding of the antibody to peptides comprising LAP-TGFβ1 fragments. A series of overlapping peptides encompassing the LAP-TGFβ1 sequence may be synthesized and screened for binding, e.g. in a direct ELISA, a competitive ELISA (where the peptide is assessed for its ability to prevent binding of an antibody to LAP-TGFβ1 bound to a well of a microtiter plate), or on a chip. Such peptide screening methods may not be capable of detecting some discontinuous functional epitopes.

An epitope may also be identified by MS-based protein foot printing, such as HDX-MS and Fast Photochemical Oxidation of Proteins (FPOP), structural methods such as X-ray crystal structure determination, molecular modeling, and nuclear magnetic resonance spectroscopy.

Single particle cryo electron microscopy (SP-Cryo-EM) can also be used to identify the epitope to which an antibody or antigen binding fragment thereof binds. SP-Cryo-EM is a technique for macromolecular structure analysis which uses a high intensity electron beam to image biological specimens in their native environment at cryogenic temperature. In recent years, SP-cryo-EM has emerged as a complementary technique to crystallography and NMR for determining near-atomic level structures suitable for application in drug discovery (Renaud et al. Nat Rev Drug Discov 2018; 17:471-92; Scapin et al. Cell Chem Biol 2018; 25:1318-25; Ceska et al. Biochemical Society Transactions 2019: p. BST20180267). In addition to high resolution information, SP-Cryo-EM has the further advantage of allowing access to larger and more complex biological systems, with the possibility of characterizing multiple conformational or compositional solution states from the same sample, providing insights into more biologically relevant states of the macromolecule. For imaging, a small volume of sample (e.g., 3 μl aliquot) is applied onto a grid and flash-frozen in a liquid ethane bath. The frozen grid is then loaded into the microscope and hundreds to thousands of images of different areas of the grids are collected. These images contain two-dimensional projections of the biological macromolecule (particles): using mathematical tools and GPU powered algorithms, the particles are identified, extracted, and classified; in the subsequent step, the different classes are used to compute one or more 3D reconstructions, corresponding to different conformations, oligomerization or binding states if they coexist in the same sample. The individual reconstructions can then be refined to high resolution.

### III. Nucleic Acid Molecules

Also provided herein are nucleic acid molecules that encode the anti-LAP antibodies or antigen binding fragments thereof described herein. The nucleic acids may be present in whole cells, in a cell lysate, or in a partially purified or substantially pure form. A nucleic acid described herein can be, for example, DNA or RNA and may or may not contain intronic sequences. In certain embodiments, the nucleic acid is a cDNA molecule. The nucleic acids described herein can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

In some embodiments, provided herein are nucleic acid molecules that encode the VH and/or VL sequences, or heavy and/or light chain sequences, of any of the anti-LAP antibodies or antigen binding fragments thereof described herein. Host cells comprising the nucleotide sequences (e.g., nucleic acid molecules) described herein are encompassed herein. Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CH1, CH2 and/or CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

Also provided herein are nucleic acid molecules with conservative substitutions that do not alter the resulting amino acid sequence upon translation of the nucleic acid molecule.

Iv. Methods of Production

Monoclonal antibodies can be prepared using a wide variety of techniques known in the art including the use of hybridoma, recombinant, and phage display technologies, or a combination thereof.

Various methods for making monoclonal antibodies described herein are available in the art. For example, the monoclonal antibodies can be made using the hybridoma method first described by Kohler et al., Nature, 256:495 (1975), or any later developments thereof, or by recombinant DNA methods (U.S. Pat. No. 4,816,567). For example, monoclonal antibodies can be produced using hybridoma techniques including those known in the art and taught, for example, in Harlow et al, Antibodies: A Laboratory Manual, (Cold Spring Harbor Laboratory Press, 2nd ed., 1988); Hammer-ling, et al., in: Monoclonal Antibodies and T-Cell Hybridomas 563-681 (Elsevier, N.Y., 1981) (said references incorporated by reference in their entireties). Methods for producing and screening for specific antibodies using hybridoma technology are routine and well known in the art. In another example, antibodies useful in the methods and compositions described herein can also be generated using various phage display methods known in the art, such as isolation from antibody phage libraries generated using the techniques described in McCafferty et al., Nature, 348:552-554 (1990). Clackson et al., Nature, 352:624-628 (1991) and Marks et al., J. Mol. Biol, 222:581-597 (1991) describe the isolation of murine and human antibodies, respectively, using phage libraries. Subsequent publications describe the production of high affinity (e.g., nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

Human antibodies can be made by a variety of methods known in the art, including phage display methods described above using antibody libraries derived from human immunoglobulin sequences. See also, U.S. Pat. Nos. 4,444,887 and 4,716,111; and PCT publication numbers WO 98/46645, WO 98/50433, WO 98/24893, WO 98/16654, WO 96/34096, WO 96/33735, and WO 91/10741, the contents of which are herein incorporated by reference in their entireties. Human antibodies can also be produced using transgenic mice which express human immunoglobulin genes, and upon immunization are capable of producing a full repertoire of human antibodies in the absence of endogenous immunoglobulin production. For an overview of this technology for producing human antibodies, see, Lonberg and Huszar, 1995, Int. Rev. Immunol. 13:65-93. Phage display technology (McCafferty et al., Nature 348:552-553 (1990)) also can be used to produce human antibodies and antibody binding fragments thereof in vitro, from immunoglobulin variable (V) domain gene repertoires from unimmunized donors. Human antibodies can also be generated by in vitro activated B cells (see U.S. Pat. Nos. 5,567,610 and 5,229,275, the contents of which are herein incorporated by reference in their entireties). Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

Chimeric antibodies can be prepared based on the sequence of a murine monoclonal antibody. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.).

Humanized forms of anti-LAP antibodies (e.g., humanized affinity matured forms of mouse anti-LAP antibodies) are chimeric antibodies which contain minimal sequence derived from non-human immunoglobulin. Humanized antibodies are typically human immunoglobulins (recipient antibody) in which residues from a CDR or hypervariable region of the recipient are replaced by residues from a CDR or hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin consensus sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The framework and CDR regions of a humanized antibody need not correspond precisely to the parental sequences, e.g., the donor antibody CDR or the consensus framework can be mutagenized by substitution, insertion and/or deletion of at least one amino acid residue so that the CDR or framework residue at that site does not correspond exactly to either the donor antibody or the consensus framework. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see e.g., Winnaker, From Genes to Clones (Veriagsgesellschaft, Weinheim, Germany 1987). In a family of immunoglobulins, each position in the consensus sequence is occupied by the amino acid occurring most frequently at that position in the family. Where two amino acids occur equally frequently, either can be included in the consensus sequence. As used herein, “Vernier zone” refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and can impact on the structure of CDRs and the affinity of the antibody. Human immunoglobulin (Ig) sequences that can be used as a recipient are well known in the art.

Framework residues in the human framework regions can be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, including, but not limited to, those described in Jones et al., Nature 321:522 (1986); Verhoeyen et al., Science 239: 1534 (1988), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Padlan, Molecular Immunology 28(4/5):489-498 (1991); Studnicka et al., Protein Engineering 7(6):805-814 (1994); Roguska. et al., PNAS 91:969-973 (1994); PCT publication number WO 91/09967, PCT/: US98/16280, US96/18978, US91/09630, US91/05939, US94/01234, GB89/01334, GB91/01134, GB92/01755; WO90/14443, WO90/14424, WO90/14430, EP 229246, EP 592,106; EP 519,596, EP 239,400, U.S. Pat. Nos. 5,565,332, 5,723,323, 5,976,862, 5,824,514, 5,817,483, 5,814,476, 5,763,192, 5,723,323, 5,766,886, 5,714,352, 6,204,023, 6,180,370, 5,693,762, 5,530, 101, 5,585,089, 5,225,539; 4,816,567, each entirely incorporated herein by reference.

The anti-LAP antibodies generated using the methods described above can be tested for desired functions, such as particular binding specificities, binding affinities, targeted cell populations, using methods known in the art and described in the Examples, for example, art-recognized protein-protein binding assays, biochemical screening assays, immunoassays, and cell-based assays. An aspect of the invention provides molecules that may be used to screen for an antibody or antigen binding fragment thereof that binds LAP, a complex comprising LAP, and/or a complex comprising LAP-TGFβ1. For example, the molecules in Table 2 or Table 3 or molecules having the amino acid sequence of any of SEQ ID NO: 1 or variants thereof are used to screen or determine binding of at least one binding protein. In various embodiments, the at least one molecule in Table 2 and/or Table 3 are used to screen or determine binding of at least one antibody or antigen binding fragment thereof.

Exemplary assays include, but are not limited to, immunoprecipitation or by an in vitro binding assay, such as radioimmunoassay (RIA), FACS, enzyme-linked immunoabsorbent assay (ELISA), bio-layer interferometry (e.g., ForteBio assay), and Scatchard analysis.

### Antibody Engineering

Further included are embodiments in which the anti-LAP antibodies or antigen-binding fragments thereof described herein (e.g., antibodies or antigen binding fragments thereof in Tables 4, 6, 8, 11-43, and 45) are engineered antibodies to include modifications to framework residues within the variable domains of the parental monoclonal antibody, e.g., to improve the properties of the antibody or antigen binding fragment thereof. Typically, such framework modifications are made to decrease the immunogenicity of the antibody or antigen binding fragment thereof. This is usually accomplished by replacing non-CDR residues in the variable domains (i.e., framework residues) in a parental (e.g., rodent) antibody or antigen binding fragment thereof with analogous residues from the immune repertoire of the species in which the antibody is to be used, e.g., human residues in the case of human therapeutics. Such an antibody or antigen binding fragment thereof is referred to as a “humanized” antibody or antigen binding fragment thereof. In some cases, it is desirable to increase the affinity, or alter the specificity of an engineered (e.g., humanized) antibody. One approach is to “backmutate” one or more framework residues to the corresponding germline sequence. More specifically, an antibody or antigen binding fragment thereof that has undergone somatic mutation can contain framework residues that differ from the germline sequence from which the antibody is derived. Such residues can be identified by comparing the antibody or antigen binding fragment thereof framework sequences to the germline sequences from which the antibody or antigen binding fragment thereof is derived. Another approach is to revert to the original parental (e.g., rodent) residue at one or more positions of the engineered (e.g. humanized) antibody, e.g. to restore binding affinity that may have been lost in the process of replacing the framework residues. (See, e.g., U.S. Pat. Nos. 5,693,762, 5,585,089 and 5,530,101.)

In certain embodiments, the anti-LAP antibodies and antigen binding fragments thereof are engineered (e.g., humanized) to include modifications in the framework and/or CDRs to improve their properties. Such engineered changes can be based on molecular modeling. A molecular model for the variable region for the parental (non-human) antibody sequence can be constructed to understand the structural features of the antibody and used to identify potential regions on the antibody that can interact with the antigen. Conventional CDRs are based on alignment of immunoglobulin sequences and identifying variable regions. Kabat et al., (1991) Sequences of Proteins of Immunological Interest, Kabat, et al.; National Institutes of Health, Bethesda, Md.; 5^{th} ed.; NIH Publ. No. 91-3242; Kabat (1978) Adv. Prot. Chem. 32:1-75; Kabat, et al., (1977) J. Biol. Chem. 252:6609-6616. Chothia and coworkers carefully examined conformations of the loops in crystal structures of antibodies and proposed hypervariable loops. Chothia, et al., (1987) J Mol. Biol. 196:901-917 or Chothia, et al., (1989) Nature 342:878-883. There are variations between regions classified as “CDRs” and “hypervariable loops”. Later studies (Raghunathan et al., (2012) J. Mol Recog. 25, 3, 103-113) analyzed several antibody-antigen crystal complexes and observed that the antigen binding regions in antibodies do not necessarily conform strictly to the “CDR” residues or “hypervariable” loops. The molecular model for the variable region of the non-human antibody can be used to guide the selection of regions that can potentially bind to the antigen. In practice, the potential antigen binding regions based on model differ from the conventional “CDR” s or “hyper variable” loops. Commercial scientific software such as MOE(Chemical Computing Group) can be used for molecular modeling. Human frameworks can be selected based on best matches with the non-human sequence both in the frameworks and in the CDRs. For FR4 (framework 4) in VH, VJ regions for the human germlines are compared with the corresponding non-human region. In the case of FR4 (framework 4) in VL, J-kappa and J-Lambda regions of human germline sequences are compared with the corresponding non-human region. Once suitable human frameworks are identified, the CDRs are grafted into the selected human frameworks. In some cases, certain residues in the VL-VH interface can be retained as in the non-human (parental) sequence. Molecular models can also be used for identifying residues that can potentially alter the CDR conformations and hence binding to antigen. In some cases, these residues are retained as in the non-human (parental) sequence. Molecular models can also be used to identify solvent exposed amino acids that can result in unwanted effects such as glycosylation, deamidation and oxidation. Developability filters can be introduced early on in the design stage to eliminate/minimize these potential problems.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Pat. No. 7,125,689.

In particular embodiments, it will be desirable to change certain amino acids containing exposed side-chains to another amino acid residue in order to provide for greater chemical stability of the final antibody, so as to avoid deamidation or isomerization. The deamidation of asparagine may occur on NG, DG, NG, NS, NA, NT, QG or QS sequences and result in the creation of an isoaspartic acid residue that introduces a kink into the polypeptide chain and decreases its stability (isoaspartic acid effect). Isomerization can occur at DG, DS, DA or DT sequences. In certain embodiments, the antibodies of the present disclosure do not contain deamidation or asparagine isomerism sites. For example, an asparagine (Asn) residue may be changed to Gln or Ala to reduce the potential for formation of isoaspartate at any Asn-Gly sequences, particularly within a CDR.

A similar problem may occur at an Asp-Gly sequence. Reissner and Aswad (2003) Cell. Mol. Life Sci. 60:1281. Isoaspartate formation may debilitate or completely abrogate binding of an antibody to its target antigen. See, Presta (2005) J. Allergy Clin. Immunol. 116:731 at 734. In various embodiment, the asparagine is changed to glutamine (Gln). It may also be desirable to alter an amino acid adjacent to an asparagine (Asn) or glutamine (Gln) residue to reduce the likelihood of deamidation, which occurs at greater rates when small amino acids occur adjacent to asparagine or glutamine. See, Bischoff & Kolbe (1994) J. Chromatog. 662:261. In addition, any methionine residues (typically solvent exposed Met) in CDRs may be changed to Lys, Leu, Ala, or Phe or other amino acids in order to reduce the possibility that the methionine sulfur would oxidize, which could reduce antigen binding affinity and also contribute to molecular heterogeneity in the final antibody preparation. Id. Additionally, in order to prevent or minimize potential scissile Asn-Pro peptide bonds, it may be desirable to alter any Asn-Pro combinations found in a CDR to Gln-Pro, Ala-Pro, or Asn-Ala. Antibodies with such substitutions are subsequently screened to ensure that the substitutions do not decrease the affinity or specificity of the antibody for LAP, or other desired biological activity to unacceptable levels.

### Antibody Engineering of the Fc Region

The antibodies (e.g., humanized antibodies) and antigen binding fragments thereof disclosed herein (e.g., antibody 20E6 and humanized affinity matured versions thereof) can also be engineered to include modifications within the Fc region, typically to alter one or more properties of the antibody, such as serum half-life, complement fixation, Fc receptor binding, and/or effector function (e.g., antigen-dependent cellular cytotoxicity). Furthermore, the antibodies and antigen binding fragments thereof disclosed herein (e.g., antibody 20E6 and humanized affinity matured versions thereof) can be chemically modified (e.g., one or more chemical moieties can be attached to the antibody) or be modified to alter its glycosylation, again to alter one or more properties of the antibody or antigen binding fragment thereof. Each of these embodiments is described in further detail below. The numbering of residues in the Fc region is that of the EU index of Kabat.

The antibodies and antigen binding fragments thereof disclosed herein (e.g., antibody 20E6 and humanized affinity matured versions thereof) also include antibodies and antigen binding fragments thereof with modified (or blocked) Fc regions to provide altered effector functions. See, e.g., U.S. Pat. No. 5,624,821; and PCT Publication numbers WO2003/086310; WO2005/120571; WO2006/0057702. Such modifications can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc regions. Changes to the Fc can also alter the half-life of antibodies in therapeutic antibodies, enabling less frequent dosing and thus increased convenience and decreased use of material. See Presta (2005) J. Allergy Clin. Immunol. 116:731 at 734-35.

In one embodiment, the antibody or antigen binding fragment thereof of the invention (e.g., antibody 20E6 and humanized affinity matured versions thereof) is an IgG4 isotype antibody or antigen binding fragment thereof comprising a Serine to Proline mutation at a position corresponding to position 228 (S228P; EU index) in the hinge region of the heavy chain constant region. This mutation has been reported to abolish the heterogeneity of inter-heavy chain disulfide bridges in the hinge region (Angal et al. supra; position 241 is based on the Kabat numbering system).

In one embodiment of the invention, the hinge region of CH1 is modified such that the number of cysteine residues in the hinge region is increased or decreased. This approach is described further in U.S. Pat. No. 5,677,425. The number of cysteine residues in the hinge region of CH1 is altered, for example, to facilitate assembly of the light and heavy chains or to increase or decrease the stability of the antibody.

In another embodiment, the Fc hinge region of an antibody or antigen binding fragment thereof of the invention (e.g., antibody 20E6 and humanized affinity matured versions thereof) is mutated to decrease the biological half-life of the antibody or antigen binding fragment thereof. More specifically, one or more amino acid mutations are introduced into the CH2-CH3 domain interface region of the Fc-hinge fragment such that the antibody or antigen binding fragment thereof has impaired Staphylococcyl protein A (SpA) binding relative to native Fc-hinge domain SpA binding. This approach is described in further detail in U.S. Pat. No. 6,165,745.

In another embodiment, the antibody or antigen binding fragment thereof of the invention (e.g., antibody 20E6 and humanized affinity matured versions thereof) is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022.

In yet other embodiments, the Fc region is altered by replacing at least one amino acid residue with a different amino acid residue to alter the effector function(s) of the antibody or antigen binding fragment thereof. For example, one or more amino acids selected from amino acid residues 234, 235, 236, 237, 297, 318, 320 and 322 can be replaced with a different amino acid residue such that the antibody has an altered affinity for an effector ligand and retains the antigen binding ability of the parent antibody. The effector ligand to which affinity is altered can be, for example, an Fc receptor or the C1 component of complement. This approach is described in further detail in U.S. Pat. Nos. 5,624,821 and 5,648,260.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551.

In another example, one or more amino acid residues within amino acid positions 231 and 239 are altered to thereby alter the ability of the antibody to fix complement. This approach is described further in PCT Publication number WO 94/29351.

In yet another example, the Fc region is modified to decrease the ability of the antibody or antigen binding fragment thereof of the invention (e.g., antibody 20E6 and humanized affinity matured versions thereof) to mediate antibody dependent cellular cytotoxicity (ADCC) and/or to decrease the affinity of the antibody or antigen binding fragment thereof for an Fc7 receptor by modifying one or more amino acids at the following positions: 238, 239, 243, 248, 249, 252, 254, 255, 256, 258, 264, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication number WO 00/42072. Moreover, the binding sites on human IgG1 for FcγR1, FcγRII, FcγRIII and FcRn have been mapped and variants with improved binding have been described (see Shields et al. (2001) J. Biol. Chem. 276:6591-6604).

In one embodiment of the invention, the Fc region is modified to decrease the ability of the antibody of the invention (e.g., antibody 20E6 and humanized affinity matured versions thereof) to mediate effector function and/or to increase anti-inflammatory properties by modifying residues 243 and 264. In one embodiment, the Fc region of the antibody or antigen binding fragment thereof is modified by changing the residues at positions 243 and 264 to alanine. In one embodiment, the Fc region is modified to decrease the ability of the antibody or antigen binding fragment thereof to mediate effector function and/or to increase anti-inflammatory properties by modifying residues 243, 264, 267 and 328.

### Altered Effector Function

In some embodiments, the Fc region of an anti-LAP antibody is modified to increase or reduce the ability of the antibody or antigen binding fragment thereof to mediate effector function and/or to increase/decrease their binding to the Fc gamma receptors (FcγRs).

The interaction between the constant region of an antigen binding protein and various Fc receptors (FcR) including FcgammaRI (CD64), FcgammaRII (CD32) and FcgammaRIII (CD16) is believed to mediate the effector functions, such as ADCC and CDC, of the antigen binding protein. The Fc receptor is also important for antibody cross-linking, which can be important for anti-tumor immunity.

Effector function can be measured in a number of ways including for example via binding of the FcgammaRIII to Natural Killer cells or via FcgammaRI to monocytes/macrophages to measure for ADCC effector function. For example, an antigen binding protein of the present invention can be assessed for ADCC effector function in a Natural Killer cell assay. Examples of such assays can be found in Shields et al., 2001 J. Biol. Chem., Vol. 276, p 6591-6604; Chappel et al., 1993 J. Biol. Chem., Vol 268, p 25124-25131; Lazar et al., 2006 PNAS, 103; 4005-4010.

Human IgG1 constant regions containing specific mutations or altered glycosylation on residue Asn297 have been shown to reduce binding to Fc receptors. In other cases, mutations have also been shown to enhance ADCC and CDC (Lazar et al. PNAS 2006, 103; 4005-4010; Shields et al. J Biol Chem 2001, 276; 6591-6604; Nechansky et al. Mol Immunol, 2007, 44; 1815-1817).

In one embodiment of the present invention, such mutations are in one or more of positions selected from 239, 332 and 330 (IgG1), or the equivalent positions in other IgG isotypes. Examples of suitable mutations are S239D and 1332E and A330L. In one embodiment, the antigen binding protein of the invention herein described is mutated at positions 239 and 332, for example S239D and 1332E or in a further embodiment it is mutated at three or more positions selected from 239 and 332 and 330, for example S239D and 1332E and A330L. (EU index numbering).

In an alternative embodiment of the present invention, there is provided an antibody comprising a heavy chain constant region with an altered glycosylation profile such that the antigen binding protein has enhanced effector function. For example, wherein the antibody has enhanced ADCC or enhanced CDC or wherein it has both enhanced ADCC and CDC effector function. Examples of suitable methodologies to produce antigen binding proteins with an altered glycosylation profile are described in PCT Publication numbers WO2003011878 and WO2006014679 and European patent number EP1229125.

In a further aspect, the present invention provides “non-fucosylated” or “afucosylated” antibodies. Non-fucosylated antibodies harbor a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcgammaRIIIa binding capacity.

The present invention also provides a method for the production of an antibody according to the invention comprising the steps of: a) culturing a recombinant host cell comprising an expression vector comprising the isolated nucleic acid as described herein, wherein the recombinant host cell does not comprise an alpha-1,6-fucosyltransferase; and b) recovering the antigen binding protein (e.g., antibody or antigen binding fragment thereof). The recombinant host cell may not normally contain a gene encoding an alpha-1,6-fucosyltransferase (for example yeast host cells such as Pichia sp.) or may have been genetically modified to inactivate an alpha-1,6-fucosyltransferase. Recombinant host cells which have been genetically modified to inactivate the FUT8 gene encoding an alpha-1,6-fucosyltransferase are available. See, e.g., the POTELLIGENT™ technology system available from BioWa, Inc. (Princeton, N.J.) in which CHOK1SV cells lacking a functional copy of the FUT8 gene produce monoclonal antibodies having enhanced antibody dependent cell mediated cytotoxicity (ADCC) activity that is increased relative to an identical monoclonal antibody produced in a cell with a functional FUT8 gene. Aspects of the POTELLIGENT™ technology system are described in U.S. Pat. Nos. U.S. Pat. Nos. 7,214,775 and 6,946,292, and PCT Publication numbers WO0061739 and WO0231240. Those of ordinary skill in the art will also recognize other appropriate systems.

It will be apparent to those skilled in the art that such modifications may not only be used alone but may be used in combination with each other in order to further enhance or decrease effector function.

Production of Antibodies with Modified Glycosylation

In still another embodiment, the antibodies or antigen binding fragments thereof of the invention (e.g., antibody and antigen binding fragments in Tables 4, 6, 8, 11-43, and 45 and variants thereof) comprise a particular glycosylation pattern. For example, an afucosylated or an aglycosylated antibody or antigen binding fragment thereof can be made (i.e., the antibody lacks fucose or glycosylation, respectively). The glycosylation pattern of an antibody or antigen binding fragment thereof may be altered to, for example, increase the affinity or avidity of the antibody or fragment for a LAP antigen. Such modifications can be accomplished by, for example, altering one or more of the glycosylation sites within the antibody or antigen binding fragment thereof sequence. For example, one or more amino acid substitutions can be made that result in removal of one or more of the variable region framework glycosylation sites to thereby eliminate glycosylation at that site. Such aglycosylation may increase the affinity or avidity of the antibody or antigen binding fragment thereof for antigen. See, e.g., U.S. Pat. Nos. 5,714,350 and 6,350,861.

Antibodies and antigen binding antigen binding fragments thereof disclosed herein may further include those produced in lower eukaryote host cells, in particular fungal host cells such as yeast and filamentous fungi have been genetically engineered to produce glycoproteins that have mammalian- or human-like glycosylation patterns (See for example, Choi et al, (2003) Proc. Natl. Acad. Sci. 100: 5022-5027; Hamilton et al., (2003) Science 301: 1244-1246; Hamilton et al., (2006) Science 313: 1441-1443; Nett et al., Yeast 28(3):237-52 (2011); Hamilton et al., Curr Opin Biotechnol. Oct;18(5):387-92 (2007)). A particular advantage of these genetically modified host cells over currently used mammalian cell lines is the ability to control the glycosylation profile of glycoproteins that are produced in the cells such that compositions of glycoproteins can be produced wherein a particular N-glycan structure predominates (see, e.g., U.S. Pat. Nos. 7,029,872 and 7,449,308). These genetically modified host cells have been used to produce antibodies that have predominantly particular N-glycan structures (See for example, Li et al., (2006) Nat. Biotechnol. 24: 210-215).

In particular embodiments, the antibodies and antigen binding fragments thereof disclosed herein (e.g., antibody and antigen binding fragments in Tables 4, 6, 8, and 11-43 and variants thereof) further include those produced in lower eukaryotic host cells and which comprise fucosylated and non-fucosylated hybrid and complex N-glycans, including bisected and multiantennary species, including but not limited to N-glycans such as GlcNAc₍₁₋₄₎Man₃GlcNAc₂; Gal₍₁₋₄₎GlcNAc₍₁₋₄₎Man₃GlcNAc₂; NANA₍₁₋₄₎Gal₍₁₋₄₎GlcNAc₍₁₋₄₎Man₃GlcNAc₂.

In particular embodiments, the antibodies and antigen binding fragments thereof provided herein may comprise antibodies or antigen binding fragments thereof having at least one hybrid N-glycan selected from the group consisting of GlcNAcMan₅GlcNAc₂; GalGlcNAcMan₅GlcNAc₂; and NANAGalGlcNAcMan₅GlcNAc₂. In particular aspects, the hybrid N-glycan is the predominant N-glycan species in the composition.

In particular embodiments, the antibodies and antigen binding fragments thereof provided herein comprise antibodies and antigen binding fragments thereof having at least one complex N-glycan selected from the group consisting of GlcNAcMan₃GlcNAc₂; GalGlcNAcMan₃GlcNAc₂; NANAGalGlcNAcMan₃GlcNAc₂; GlcNAc₂Man₃GlcNAc₂; GalGlcNAc₂Man₃GlcNAc₂; Gal₂GlcNAc₂Man₃GlcNAc₂; NANAGal₂GlcNAc₂Man₃GlcNAc₂; and NANA₂Gal₂GlcNAc₂Man₃GlcNAc₂. In particular aspects, the complex N-glycan are the predominant N-glycan species in the composition. In further aspects, the complex N-glycan is a particular N-glycan species that comprises about 30%, 40%, 50%, 60%, 70%, 80%, 90%, 95%, 97%, 98%, 99%, or 100% of the complex N-glycans in the composition. In one embodiment, the antibody and antigen binding fragments thereof provided herein comprise complex N-glycans, wherein at least 50%, 60%, 70%, 80%, 90%, 95%, 97%, 98%, 99%, or 100% of the complex N-glycans comprise the structure NANA₂Gal₂GlcNAc₂Man₃GlcNAc₂, wherein such structure is afucosylated. Such structures can be produced, e.g., in engineered Pichia pastoris host cells and CHO cells.

In particular embodiments, the N-glycan is fucosylated. In general, the fucose is in an α1,3-linkage with the GlcNAc at the reducing end of the N-glycan, an α1,6-linkage with the GlcNAc at the reducing end of the N-glycan, an α1,2-linkage with the Gal at the non-reducing end of the N-glycan, an α1,3-linkage with the GlcNac at the non-reducing end of the N-glycan, or an α1,4-linkage with a GlcNAc at the non-reducing end of the N-glycan. Therefore, in particular aspects of the above the glycoprotein compositions, the glycoform is in an α1,3-linkage or α1,6-linkage fucose to produce a glycoform selected from the group consisting of Man₅GlcNAc₂(Fuc), GlcNAcMan₅GlcNAc₂(Fuc), Man₃GlcNAc₂(Fuc), GlcNAcMan₃GlcNAc₂(Fuc), GlcNAc₂Man₃GlcNAc₂(Fuc), GalGlcNAc₂Man₃GlcNAc₂(Fuc), Gal₂GlcNAc₂Man₃GlcNAc₂(Fuc), NANAGal₂GlcNAc₂Man₃GlcNAc₂(Fuc), and NANA₂Gal₂GlcNAc₂Man₃GlcNAc₂(Fuc); in an α1,3-linkage or α1,4-linkage fucose to produce a glycoform selected from the group consisting of GlcNAc(Fuc)Man₅GlcNAc₂, GlcNAc(Fuc)Man₃GlcNAc₂, GlcNAc₂(Fuc₁₋₂)Man₃GlcNAc₂, GalGlcNAc₂(Fuc₁₋₂)Man₃GlcNAc₂, Gal₂GlcNAc₂(Fuc₁₋₂)Man₃GlcNAc₂, NANAGal₂GlcNAc₂(Fuc₁₋₂)Man₃GlcNAc₂, and NANA₂Gal₂GlcNAc₂(Fuc₁₋₂)Man₃GlcNAc₂; or in an α1,2-linkage fucose to produce a glycoform selected from the group consisting of Gal(Fuc)GlcNAc₂Man₃GlcNAc₂, Gal₂(Fuc₁₋₂)GlcNAc₂Man₃GlcNAc₂, NANAGal₂(Fuc₁₋₂)GlcNAc₂Man₃GlcNAc₂, and NANA₂Gal₂(Fuc₁₋₂)GlcNAc₂Man₃GlcNAc₂.

In further aspects, the antibodies (e.g., humanized antibodies) or antigen binding fragments thereof comprise high mannose N-glycans, including but not limited to, Man₈GlcNAc₂, Man₇GlcNAc₂, Man₆GlcNAc₂, Man₅GlcNAc₂, Man₄GlcNAc₂, or N-glycans that consist of the Man₃GlcNAc₂ N-glycan structure.

In further aspects of the above, the complex N-glycans further include fucosylated and non-fucosylated bisected and multiantennary species.

As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs co-translationally in the lumen of the ER and continues post-translationally in the Golgi apparatus for N-linked glycoproteins. N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). Usually, N-glycan structures are presented with the non-reducing end to the left and the reducing end to the right. The reducing end of the N-glycan is the end that is attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms”.

With respect to complex N-glycans, the terms “G-2”, “G-1”, “GO”, “G1”, “G2”, “A1”, and “A2” mean the following. “G-2” refers to an N-glycan structure that can be characterized as Man₃GlcNAc₂; the term “G-1” refers to an N-glycan structure that can be characterized as GlcNAcMan₃GlcNAc₂; the term “GO” refers to an N-glycan structure that can be characterized as GlcNAc₂Man₃GlcNAc₂; the term “G1” refers to an N-glycan structure that can be characterized as GalGlcNAc₂Man₃GlcNAc₂; the term “G2” refers to an N-glycan structure that can be characterized as Gal₂GlcNAc₂Man₃GlcNAc₂; the term “A1” refers to an N-glycan structure that can be characterized as NANAGal₂GlcNAc₂Man₃GlcNAc₂; and, the term “A2” refers to an N-glycan structure that can be characterized as NANA₂Gal₂GlcNAc₂Man₃GlcNAc₂. Unless otherwise indicated, the terms G-2”, “G-1”, “GO”, “G1”, “G2”, “A1”, and “A2” refer to N-glycan species that lack fucose attached to the GlcNAc residue at the reducing end of the N-glycan. When the term includes an “F”, the “F” indicates that the N-glycan species contains a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that produce fucose.

With respect to multiantennary N-glycans, the term “multiantennary N-glycan” refers to N-glycans that further comprise a GlcNAc residue on the mannose residue comprising the non-reducing end of the 1,6 arm or the 1,3 arm of the N-glycan or a GlcNAc residue on each of the mannose residues comprising the non-reducing end of the 1,6 arm and the 1,3 arm of the N-glycan. Thus, multiantennary N-glycans can be characterized by the formulas GlcNAc₍₂₋₄₎Man₃GlcNAc₂, Gal₍₁₋₄₎GlcNAc₍₂₋₄₎Man₃GlcNAc₂, or NANA₍₁₋₄₎Gal₍₁₋₄₎GlcNAc₍₂₋₄₎Man₃GlcNAc₂. The term “1-4” refers to 1, 2, 3, or 4 residues. With respect to bisected N-glycans, the term “bisected N-glycan” refers to N-glycans in which a GlcNAc residue is linked to the mannose residue at the reducing end of the N-glycan. A bisected N-glycan can be characterized by the formula GlcNAc₃Man₃GlcNAc₂ wherein each mannose residue is linked at its non-reducing end to a GlcNAc residue. In contrast, when a multiantennary N-glycan is characterized as GlcNAc₃Man₃GlcNAc₂, the formula indicates that two GlcNAc residues are linked to the mannose residue at the non-reducing end of one of the two arms of the N-glycans and one GlcNAc residue is linked to the mannose residue at the non-reducing end of the other arm of the N-glycan.

### Antibody Physical Properties

The antibodies and antigen binding fragments thereof disclosed herein (e.g., antibody and antigen binding fragments in Tables 4, 6, 8, 11-43, 45 and variants thereof) may further contain one or more glycosylation sites in either the light or heavy chain immunoglobulin variable region. Such glycosylation sites may result in increased immunogenicity of the antibody or antigen binding fragment thereof or an alteration of the PK of the antibody due to altered antigen binding (Marshall et al. (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al. (2000) Mol Immunol 37:697-706). Glycosylation has been known to occur at motifs containing an N—X-S/T sequence.

Each antibody or antigen binding fragment thereof will have a characteristic melting temperature, with a higher melting temperature indicating greater overall stability in vivo (Krishnamurthy R and Manning M C (2002) Curr Pharm Biotechnol 3:361-71). In general, the T_{M1} (the temperature of initial unfolding) may be greater than 60° C., greater than 65° C., or greater than 70° C. The melting point of an antibody or antigen binding fragment thereof can be measured using differential scanning calorimetry (Chen et al (2003) Pharm Res 20:1952-60; Ghirlando et al (1999) Immunol Lett 68:47-52) or circular dichroism (Murray et al. (2002) J. Chromatogr Sci 40:343-9). In a further embodiment, antibodies and antigen binding fragments thereof (e.g., antibody 20E6 and humanized versions thereof) are selected that do not degrade rapidly. Degradation of an antibody or antigen binding fragment thereof can be measured using capillary electrophoresis (CE) and MALDI-MS (Alexander A J and Hughes D E (1995) Anal Chem 67:3626-32).

In a further embodiment, antibodies and antigen binding fragments thereof are selected that have minimal aggregation effects, which can lead to the triggering of an unwanted immune response and/or altered or unfavorable pharmacokinetic properties. Generally, antibodies and antigen binding fragments thereof are acceptable with aggregation of 25% or less, 20% or less, 15% or less, 10% or less, or 5% or less. Aggregation can be measured by several techniques, including size-exclusion column (SEC), high performance liquid chromatography (HPLC), and light scattering.

### V. Multispecific Antibodies

Multispecific antibodies (e.g., bispecific antibodies) provided herein include at least one binding region for a particular epitope on LAP-TGFβ1 (e.g., human LAP-TGFβ1) as described herein, and at least one other binding region (e.g., a cancer antigen). Multispecific antibodies can be prepared as full-length antibodies or antigen binding fragments thereof (e.g. F(ab′)₂ antibodies).

Methods for making multispecific antibodies are well known in the art (see, e.g., PCT Publication numbers WO 05117973 and WO 06091209). For example, production of full length multispecific antibodies can be based on the co-expression of two paired immunoglobulin heavy chain-light chains, where the two chains have different specificities. Various techniques for making and isolating multispecific antibody fragments directly from recombinant cell culture have also been described. For example, multispecific antibodies can be produced using leucine zippers. Another strategy for making multispecific antibody fragments by the use of single-chain Fv (scFv) dimers has also been reported.

Examples of suitable multispecific molecule platforms include, but are not limited to, Dual Targeting (DT)-Ig (GSK/Domantis), Two-in-one Antibody (Genentech), Cross-linked Mabs (Karmanos Cancer Center), Fcab and mAb² (F-Star), CovX-body (CovX/Pfizer), Dual Variable Domain (DVD)-Ig (Abbott), IgG-like Bispecific (ImClone/Eli Lilly), Ts2Ab (Medlmmune/AZ) and BsAb (Zymogenetics), HERCULES (Biogen Idec), TvAb (Roche), ScFv/Fc Fusions, SCORPION (Emergent BioSolutions/Trubion, Zymogenetics/BMS), Dual Affinity Retargeting Technology (Fc-DART) (MacroGenics), Dual(ScFv)2-Fab (National Research Center for Antibody Medicine—China), F(ab)2 (Medarex/AMGEN), Dual-Action or Bis-Fab (Genentech), Dock-and-Lock (DNL) (ImmunoMedics), Bivalent Bispecific (Biotecnol), SEED (EMD Serono), mAb² (F-star), Fab-Fv (UCB-Celltech), Bispecific T Cell Engager (BiTE) (Micromet, Tandem Diabody (Tandab) (Affimed), Dual Affinity Retargeting Technology (DART) (MacroGenics), Single-chain Diabody (Academic), TCR-like Antibodies (AIT, ReceptorLogics), COMBODY (Epigen Biotech), dual targeting nanobodies (Ablynx), and Fc-engineered IgG1 (Xencor).

In a particular embodiment, the multispecific antibody comprises a first antibody (or binding portion thereof) which binds to LAP-TGFβ1 derivatized or linked to another functional molecule, e.g., another peptide or protein (e.g., another antibody or ligand for a receptor) to generate a multispecific molecule that binds to LAP-TGFβ1 and a non-LAP target molecule. An antibody or antigen binding fragment thereof may be derivatized or linked to more than one other functional molecule to generate multispecific molecules that bind to more than two different binding sites and/or target molecules. To create a multispecific molecule, an antibody or antigen binding fragment thereof disclosed herein can be functionally linked (e.g., by chemical coupling, genetic fusion, noncovalent association or otherwise) to one or more other binding molecules, such as another antibody or antigen binding fragment thereof, antibody fragment, peptide, receptor, or binding mimetic, such that a multispecific molecule results.

Accordingly, multispecific molecules, for example, bispecific antibodies and bifunctional antibodies, comprising at least one first binding specificity for a particular epitope on LAP-TGFβ1 (e.g., human LAP-TGFβ1) and a second binding specificity for a second target are contemplated. In some embodiments, the second target is the second binding region specifically binds to a tumor-associated antigen. Tumor-associated antigens are well known in the art. Exemplary tumor-associated antigens include, but are not limited to, AFP, ALK, BAGE proteins, β-catenin, brc-abl, BRCA1, BORIS, CA9, carbonic anhydrase IX, caspase-8, CCR5, CD19, CD20, CD30, CDK4, CEA, cyclin-B1, CYP1B1, EGFR, EGFRvIII, ErbB2/Her2, ErbB3, ErbB4, ETV6-AML, EpCAM, EphA2, Fra-1, FOLR1, GAGE proteins (e.g., GAGE-1, -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Mucd, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, Steap-1, Steap-2, STn, survivin, TAG-72, TGF-β, TMPRSS2, Tn, TRP-1, TRP-2, tyrosinase, and uroplakin-3.

In some embodiments, the second binding region of the bispecific antibody specifically binds to CD4, CD8, CD45, CD56, CD14, CD16, CD19, CD20, CD25, CD38, CD11b, CD22, CD30, CD39, CD 114, CD23, CD73, CD163, CD206, CD203, CD200R, PD-1, PD-L1, PD-L2, CTLA-4, IDO, TIM-3, LAG-3, TIGIT, PVR, PVRL2, B7H3, B7H4, CSF-1R, VISTA, KIR, OX-40, GITR, 4-1BB, CD40, CD40L, CD27/CD70, CD28, ICOS, CD3, CD56, NKG2DA, NKG2DB, NKG2DC, NKG2DD, NKG2DF, NKG2DH, CD94, NKP46, NKP30, CD33, CD73, CD47, LILRB1, CD91, calreticulin, CD122, GARP, LRRC33, LAP2, LAP3, TGFβ1, TGFβ2, TGFβ3, FAP, cadherin 11 and stanniocalcin 1. In some embodiments, the second binding region has agonistic properties when binding to a target, e.g., a TNF family member agonist, OX40 ligand, CD137 ligand, CD137 agonist, STING agonist, GITR agonist, ICOS agonist, and CD28 agonist.

In some embodiments, the antibody is a trispecific antibody comprising a first, second, and third binding region, wherein the first binding region comprises the binding specificity (e.g., antigen binding region) of an anti-LAP antibody described herein, and the second and third binding regions bind to two different targets (or different epitopes on the same target), for example, the targets described above.

In some embodiments, the antibody is a bifunctional antibody comprising an anti-LAP antibody described herein and a receptor molecule (i.e., a receptor trap construct such as a TGFβ superfamily ligand receptor (e.g., ActRIIB and variants thereof) or VEGFR).

In one embodiment, the multispecific molecules comprise as a binding specificity at least one antibody, or an antibody fragment thereof, including, e.g., a Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778.

The multispecific molecules can be prepared by conjugating the constituent binding specificities, e.g., the anti-FcR and anti-LAP binding specificities, using methods known in the art. For example, each binding specificity of the multispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the multispecific molecule is a mAb×mAb, mAb×Fab, Fab×F(ab′)₂ or ligand×Fab fusion protein. A multispecific molecule can be a single chain molecule comprising one single chain antibody and a binding determinant, or a single chain bispecific molecule comprising two binding determinants. Multispecific molecules may comprise at least two single chain molecules. Methods for preparing multispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the multispecific molecules to their specific targets can be confirmed by, for example, enzyme-linked immunosorbent assay (ELISA), radioimmunoassay (RIA), fluorescence-activated cell sorting (FACS) analysis, bioassay (e.g., growth inhibition), or western blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA). The radioactive isotope can be detected by such means as the use of a α γ-β counter or a scintillation counter or by autoradiography.

### VI. Immunoconjugates

Immunoconjugates comprising the anti-LAP antibodies or antigen binding fragments thereof described herein can be formed by conjugating the antibodies to another therapeutic agent to form, e.g., an antibody-drug conjugate (ADC). Suitable agents include, for example, a cytotoxic agent (e.g., a chemotherapeutic agent), a toxin (e.g. an enzymatically active toxin of bacterial, fungal, plant or animal origin, or fragments thereof), and/or a radioactive isotope (i.e., a radioconjugate). Additional suitable agents include, e.g., antimetabolites, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In some embodiments, ADCs with the anti-LAP antibodies or antigen binding fragment thereof described herein (e.g., conjugated to a cytotoxic agent) that bind to immunosuppressive cells (e.g., regulatory T cells) can be used to deplete the immunosuppressive cells from, e.g., the tumor microenvironment.

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, neomycin, and the tricothecenes. Additional examples of cytotoxins or cytotoxic agents include, e.g., taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

In the ADC, the antibody and therapeutic agent preferably are conjugated via a cleavable linker such as a peptidyl, disulfide, or hydrazone linker. More preferably, the linker is a peptidyl linker such as Val-Cit, Ala-Val, Val-Ala-Val, Lys-Lys, Pro-Val-Gly-Val-Val (SEQ ID NO: 2649), Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publication numbers WO 02/096910; WO 07/038658; WO 07/051081; WO 07/059404; WO 08/083312; and WO 08/103693; U.S. Patent Publication numbers 20060024317; 20060004081; and 20060247295; the disclosures of which are incorporated herein by reference.

A variety of radionuclides are available for the production of radioconjugated anti-LAP antibodies. Examples include ²¹²Bi, ¹³¹I, ¹³¹In ⁹⁰Y and ¹⁸⁶ Re.

Immunoconjugates can also be used to modify a given biological response, and the drug moiety is not to be construed as limited to classical chemical therapeutic agents. For example, the drug moiety may be a protein or polypeptide possessing a desired biological activity (e.g., lymphokines, tumor necrosis factor, IFNγ, growth factors).

Immunoconjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). Carbon-14-labeled 1-isothiocyanatobenzyl-3-methyldiethylene triaminepentaacetic acid (MX-DTPA) is an exemplary chelating agent for conjugation of radionucleotide to the antibody (see, e.g., PCT publication number WO94/11026).

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies ′84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

The anti-LAP antibodies or antigen binding fragments thereof described herein also are used for diagnostic purposes. Such antibodies or antigen binding fragments thereof can be conjugated to an appropriate detectable agent to form an immunoconjugate. For diagnostic purposes, appropriate agents are detectable labels that include radioisotopes, for whole body imaging, and radioisotopes, enzymes, fluorescent labels and other suitable antibody tags for sample testing.

The detectable labels can be any of the various types used currently in the field of in vitro diagnostics, including particulate labels, isotopes, chromophores, fluorescent markers, luminescent markers, metal labels (e.g., for CyTOF, imaging mass cytometry), phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.1 3,7}decan}-4-yl) phenyl phosphate (CSPD), as well as CDP and CDP-star® or other luminescent substrates well-known to those in the art, for example the chelates of suitable lanthanides such as Terbium(III) and Europium(III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

Preferably, conjugation methods result in linkages which are substantially (or nearly) non-immunogenic, e.g., peptide-(i.e. amide-), sulfide-, (sterically hindered), disulfide-, hydrazone-, and ether linkages. These linkages are nearly non-immunogenic and show reasonable stability within serum (see e.g. Senter, P. D., Curr. Opin. Chem. Biol. 13 (2009) 235-244; and PCT Publication numbers WO 2009/059278 and WO 95/17886).

Depending on the biochemical nature of the moiety and the antibody, different conjugation strategies can be employed. In case the moiety is naturally occurring or recombinant of between 50 to 500 amino acids, there are standard procedures in text books describing the chemistry for synthesis of protein conjugates, which can be easily followed by the skilled artisan (see e.g. Hackenberger, C. P. R., and Schwarzer, D., Angew. Chem. Int. Ed. Engl. 47 (2008) 10030-10074). In one embodiment the reaction of a maleinimido moiety with a cysteine residue within the antibody or the moiety is used. This is an especially suited coupling chemistry in case e.g. a Fab or Fab′-fragment of an antibody is used. Alternatively, in one embodiment coupling to the C-terminal end of the antibody or moiety is performed. C-terminal modification of a protein, e.g. of a Fab-fragment can e.g. be performed as described (Sunbul, M. and Yin, J., Org. Biomol. Chem. 7 (2009) 3361-3371).

In general, site specific reaction and covalent coupling is based on transforming a natural amino acid into an amino acid with a reactivity which is orthogonal to the reactivity of the other functional groups present. For example, a specific cysteine within a rare sequence context can be enzymatically converted in an aldehyde (see Frese, M. A., and Dierks, T., ChemBioChem. 10 (2009) 425-427). It is also possible to obtain a desired amino acid modification by utilizing the specific enzymatic reactivity of certain enzymes with a natural amino acid in a given sequence context (see, e.g., Taki, M. et al., Prot. Eng. Des. Sel. 17 (2004) 119-126; Gautier, A. et al. Chem. Biol. 15 (2008) 128-136; and Protease-catalyzed formation of C—N bonds is used by Bordusa, F., Highlights in Bioorganic Chemistry (2004) 389-403). Site specific reaction and covalent coupling can also be achieved by the selective reaction of terminal amino acids with appropriate modifying reagents. The reactivity of an N-terminal cysteine with benzonitrils (see Ren, H. et al., Angew. Chem. Int. Ed. Engl. 48 (2009) 9658-9662) can be used to achieve a site-specific covalent coupling. Native chemical ligation can also rely on C-terminal cysteine residues (Taylor, E. Vogel; Imperiali, B, Nucleic Acids and Molecular Biology (2009), 22 (Protein Engineering), 65-96).

The moiety may also be a synthetic peptide or peptide mimic. In case a polypeptide is chemically synthesized, amino acids with orthogonal chemical reactivity can be incorporated during such synthesis (see e.g. de Graaf, A. J. et al., Bioconjug. Chem. 20 (2009) 1281-1295). Since a great variety of orthogonal functional groups is at stake and can be introduced into a synthetic peptide, conjugation of such peptide to a linker is standard chemistry.

In some embodiments, the moiety attached to an anti-LAP antibody or antigen binding fragment thereof is selected from the group consisting of a detectable moiety, binding moiety, a labeling moiety, and a biologically active moiety.

### VII. Assays

The anti-LAP antibodies or antigen binding fragments thereof disclosed herein can be tested for desired properties, e.g., those described herein, using a variety of assays known in the art.

In one embodiment, the antibodies or antigen binding fragments thereof are tested for specific binding to LAP-TGFβ1 (e.g., human LAP-TGFβ1). Methods for analyzing binding affinity, cross-reactivity, and binding kinetics of various anti-LAP antibodies or antigen binding fragments thereof include standard assays known in the art, for example, Biacore™ surface plasmon resonance (SPR) analysis using a Biacore™ 2000 SPR instrument (Biacore AB, Uppsala, Sweden) or bio-layer interferometry (e.g., ForteBio assay), as described in the Examples. In some embodiments, the LAP used in the binding assay is complexed with TGFβ1. In some embodiments, the LAP used in the binding assay is not complexed with TGFβ1. In some embodiments, the LAP used in the binding assay is complexed with TGFβ1 and GARP or a fragment of GARP or LRRC33 or a fragment of LRRC33. In some embodiments the LAP used in the binding assay is complexed with TGFβ1 and LTBP (e.g., LTBP1, LTBP3, or LTBP4) or a fragment of LTBP.

In one embodiment, the antibodies or antigen binding fragments thereof are tested for the ability to bind to cells that have been transfected with LAP-TGFβ1. In some embodiments the cells have also been transfected with GARP or LRRC33.

In one embodiment, the antibodies or antigen binding fragments thereof are screened for the ability to bind to the surface of beads that have been coated with LAP.

In one embodiment, the antibodies or antigen binding fragments thereof are screened for the ability to bind to LAP on cells expressing a heparin sulfate glycoprotein such as syndecan-4. For example, heparin sulfate glycoprotein-expressing cells are incubated with LAP or with LAP complexed to LTBP (e.g., LTBP1, LTBP3, or LTBP4) and the antibodies are screened for binding by flow cytometry.

In one embodiment, the antibodies or antigen binding fragments thereof are tested for the ability to bind or affect TGFβ1. In one embodiment, the antibodies are screened for the ability to bind or affect TGFβ2. In one embodiment, the antibodies are tested for the ability to bind or affect TGFβ3.

In another embodiment, the antibodies or antigen binding fragments thereof are tested for their effects on TGFβ activation (e.g., inhibition, stimulation, or no effect). In some embodiments, TGFβ1 activation is mediated by the binding of integrins including, but not limited, to αvβ6, αvβ8, αvβ3, or αvβ1. In some embodiments, TGFβ1 activation is mediated by matrix metalloproteases including, but not limited to, MMP2 and MMP9. In some embodiments, TGFβ1 activation is mediated by thrombospondin. In some embodiments, TGFβ1 activation is mediated by serum proteases. In some embodiments, TGFβ1 activation is mediated by heat, by shear forces, by a shift in pH or by ionizing radiation. In some embodiments, TGFβ1 activation is mediated by reactive oxygen species (ROS). The source of LAP in the activation assays can be LAP on the surface of a transfected cell line, LAP on the surface of a cell population that expresses LAP endogenously or in response to specific stimuli, LAP bound to extracellular matrix, LAP in solution (e.g., recombinant LAP), either complexed with TGFβ1 or without TGFβ1 or complexed with TGFβ1 and an anchor protein, such as GARP, LRRC33, LTBP1, LTBP3, or LTBP4. LAP-TGFβ1 can be purchased from R&D Systems or can be isolated from cell supernatants. The effect an antibody has on TGFβ1 activation can be determined, for example, using an ELISA which measures levels of active TGFβ1 under different conditions (e.g., with or without antibody). The effect an antibody has on LAP-TGFβ1 activation can also be determined using a reporter cell line that expresses TGFβ receptor and responds to mature TGFβ.

In another embodiment, the antibodies or antigen binding fragments thereof are tested for the ability to bind LAP in the extracellular matrix. Suitable methods for determining whether antibodies bind to LAP in the extracellular matrix include in vitro assays, wherein cells (e.g., P3U1 cells transfected with LAP-TGFB) are cultured to lay down ECM on culture plates and subsequently removed, and labeled antibodies are tested for their ability to bind to the LAP and ECM left on the culture plate surface. Similar assays can be run using fibroblast cell lines or other cells that are known to secrete LAP-TGFβ and extracellular matrix components. In some embodiments, whether or not the anti-LAP antibodies bind to or do not bind to ECM can be determined by an ELISA, where the ECM has been shown to express latent TGFβ using commercially available antibodies.

In another embodiment, the antibodies or antigen binding fragments thereof are tested for their ability to bind to particular cell types, e.g., immune cells (e.g., immunosuppressive cells, leukocytes) or platelets. The binding of antibodies or antigen binding fragments thereof to certain leukocyte populations (e.g., Tregs, macrophages, MDSCs, GARP-negative cells) can be determined using flow cytometry.

Antibodies or antigen binding fragments thereof can also be tested for their ability to inhibit the proliferation or viability of cells (either in vivo or in vitro), such as tumor cells, using art-recognized methods (e.g., 3H-thymidine incorporation, immunohistochemistry with proliferation markers, animal cancer models).

Antibodies or antigen binding fragments thereof can also be tested for their anti-tumor activity in vivo (e.g., as monotherapy or combination therapy), using syngeneic tumor models well known in the art, such as the CT26 colorectal tumor model, EMT6 breast cancer model, and 4T1 breast cancer tumor metastasis model. See WO/2016/115345 and WO/2019/075090. Anti-LAP antibodies can also be tested in tumor xenogragft models which are known to be inhibited by anti-TGFβ antibodies (e.g., Detroit 562 tumor xenograft model).

### VIII. Compositions

Also provided herein are compositions (e.g., pharmaceutical compositions) comprising the anti-LAP antibodies or antigen binding fragments thereof described herein, immunoconjugates comprising the same, or bispecific antibodies comprising the same, and a carrier (e.g., pharmaceutically acceptable carrier). Such compositions are useful for various therapeutic applications.

In some embodiments, pharmaceutical compositions disclosed herein can include other compounds, drugs, and/or agents used for the treatment of various diseases (e.g., cancer, fibrosis, autoimmune diseases). Such compounds, drugs, and/or agents can include, for example, an anti-cancer agent, a chemotherapeutic agent, an immunosuppressive agent, an immunostimulatory agent, an immune checkpoint inhibitor, and/or an anti-inflammatory agent. Exemplary compounds, drugs, and agents that can be formulated together or separately with the anti-LAP antibodies or antigen binding fragments thereof described herein are described in the next section (i.e., Section IX; Uses and Methods).

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

The pharmaceutical compounds described herein may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition described herein may also include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. A pharmaceutical composition may comprise a preservative or may be devoid of a preservative. Supplementary active compounds can be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms described herein are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody or antigen binding fragment thereof, the dosage ranges from about 0.0001 to 100 mg/kg, and more usually 0.01 to 5 or 10 mg/kg, of the host body weight. For administration of the antibody or antigen binding fragment thereof, the dosage ranges from about 0.0001 to 100 mg. An exemplary treatment regime entails administration once per week, once every two weeks, once every three weeks, once every four weeks, once a month, once every 3 months or once every three to 6 months.

An antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions described herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The therapeutically effective dosage of an anti-LAP antibody or antigen binding fragment thereof in various embodiments results in a decrease in severity of disease symptoms, an increase in frequency and duration of disease symptom-free periods, or a prevention of impairment or disability due to the disease affliction. In the context of cancer, a therapeutically effective dose preferably results in increased survival, and/or prevention of further deterioration of physical symptoms associated with cancer. A therapeutically effective dose may prevent or delay onset of cancer, such as may be desired when early or preliminary signs of the disease are present.

A composition described herein can be administered via one or more routes of administration using one or more of a variety of methods known in the art. As will be appreciated by the skilled artisan, the route and/or mode of administration will vary depending upon the desired results. Preferred routes of administration for antibodies described herein include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody or antigen binding fragment thereof described herein can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules for use with anti-LAP antibodies described herein include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the anti-LAP antibodies or antigen binding fragments thereof described herein can be formulated to ensure proper distribution in vivo. For example, the blood-brain barrier (BBB) excludes many highly hydrophilic compounds. To ensure that the therapeutic compounds described herein cross the BBB (if desired, e.g., for brain cancers), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### IX. Uses and Methods

The antibodies, antibody compositions, and methods described herein have numerous in vitro and in vivo utilities.

For example, provided herein is a method of treating cancer comprising administering to a subject in need thereof an anti-LAP antibody or antigen binding fragment thereof described herein, such that the subject is treated, e.g., such that growth of cancerous tumors is inhibited or reduced and/or that the tumors regress and/or that prolonged survival is achieved.

In one embodiment, provided herein is a method of treating cancer comprising administering to a subject in need thereof an effective amount (e.g., a therapeutically effective amount) of an anti-LAP antibody described herein (or a bispecific antibody or immunoconjugate comprising the antibody). In some embodiments, the subject is administered a further therapeutic agent. In some embodiments, the further therapeutic agent is selected from the group consisting of: an anti-PD-1 antibody or an antigen binding fragment thereof, an anti-LAG3 antibody or an antigen biding portion thereof, an anti-VISTA antibody or an antigen binding fragment thereof, an anti-BTLA antibody or an antigen binding fragment thereof, an anti-TIM3 antibody or an antigen binding fragment thereof, an anti-CTLA4 antibody or an antigen binding fragment thereof, an anti-HVEM antibody or an antigen binding fragment thereof, an anti-CD27 antibody or an antigen binding fragment thereof, an anti-CD137 antibody or an antigen binding fragment thereof, an anti-OX40 antibody or an antigen binding fragment thereof, an anti-CD28 antibody or an antigen binding fragment thereof, an anti-PDL1 antibody or an antigen binding fragment thereof, an anti-PDL2 antibody or an antigen binding fragment thereof, an anti-GITR antibody or an antigen binding fragment thereof, an anti-ICOS antibody or an antigen binding fragment thereof, an anti-SIRPα antibody or an antigen binding fragment thereof, an anti-ILT2 antibody or an antigen binding fragment thereof, an anti-ILT3 antibody or an antigen binding fragment thereof, an anti-ILT4 antibody or an antigen binding fragment thereof, an anti-ILT5 antibody or an antigen binding fragment thereof, and an anti-4-1BB antibody or an antigen binding fragment thereof. In some embodiments, anti-PD1 antibody or antigen binding fragment thereof is pembrolizumab or an antigen biding fragment thereof. The heavy and light chain sequences of pembrolizumab are set forth in SEQ ID NOs: 2213 and 2214, respectively.

pembrolizumab heavy chain
(SEQ ID NO.: 2213)
QVQLVQSGVEVKKPGASVKVSCKASGYTFTNYYMYWVRQA
PGQGLEWMGGINPSNGGTNFNEKFKNRVTLTTDSSTTTAY
MELKSLQFDDTAVYYCARRDYRFDMGFDYWGQGTTVTVSS
ASTKGPSVFPLAPCSRSTSESTAALGCLVKDYFPEPVTVS
WNSGALTSGVHTFPAVLQSSGLYSLSSVVTVPSSSLGTKT
YTCNVDHKPSNTKVDKRVESKYGPPCPPCPAPEFLGGPSV
FLFPPKPKDTLMISRTPEVTCVVVDVSQEDPEVQFNWYVD
GVEVHNAKTKPREEQFNSTYRVVSVLTVLHQDWLNGKEYK
CKVSNKGLPSSIEKTISKAKGQPREPQVYTLPPSQEEMTK
NQVSLTCLVKGFYPSDIAVEWESNGQPENNYKTTPPVLDS
DGSFFLYSRLTVDKSRWQEGNVFSCSVMHEALHNHYTQKS
LSLSLGK
pembrolizumab light chain
(SEQ ID NO.: 2214)
EIVLTQSPATLSLSPGERATLSCRASKGVSTSGYSYLHWY
QQKPGQAPRLLIYLASYLESGVPARFSGSGSGTDFTLTIS
SLEPEDFA VYYCQHSRDLPLTFGGGTKVEIKRTVAAPSV
FIFPPSDEQLKSGTASVVCLLNNFYPREAKVQWKVDNALQ
SGNSQESVTEQDSKDSTYSLSSTLTLSKADYEKHKVYACE
VTHQGLSSPVTKSFNRGEC

In some embodiments, the further therapeutic agent is nivolumab. In various embodiments, the heavy and light chain sequences of nivolumab are set forth in comprising SEQ ID NOs. 2215 and 2216.

nivolumab heavy chain
(SEQ ID NO.: 2215)
QVQLVESGGGVVQPGRSLRLDCKASGITFSNSGMHWVRQA
PGKGLEWVAVIWYDGSKRYYADSVKGRFTISRDNSKNTLF
LQMNSLRAEDTAVYYCATNDDYWGQGTLVTVSSASTKGPS
VFPLAPCSRSTSESTAALGCLVKDYFPEPVTVSWNSGALT
SGVHTFPAVLQSSGLYSLSSVVTVPSSSLGTKTYTCNVDH
KPSNTKVDKRVESKYGPPCPPCPAPEFLGGPSVFLFPPKP
KDTLMISRTPEVTCVVVDVSQEDPEVQFNWYVDGVEVHNA
KTKPREEQFNSTYRVVSVLTVLHQDWLNGKEYKCKVSNKG
LPSSIEKTISKAKGQPREPQVYTLPPSQEEMTKNQVSLTC
LVKGFYPSDIAVEWESNGQPENNYKTTPPVLDSDGSFFLY
SRLTVDKSRWQEGNVFSCSVMHEALHNHYTQKSLSLSLGK
nivolumab light chain
(SEQ ID NO. 2216)
EIVLTQSPATLSLSPGERATLSCRASQSVSSYLAWYQQKP
GQAPRLLIYDASNRATGIPARFSGSGSGTDFTLTISSLEP
EDFAVYYCQQSSNWPRTFGQGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC

In some embodiments, the cancer is characterized by abnormal TGFβ activity. In some embodiments, the cancer is associated with fibrosis. In some embodiments, the cancer is associated with infiltration of CD4+ regulatory T cells. In some embodiments, the cancer is associated with infiltration of CD8+ regulatory T cells. In some embodiments, the cancer is associate with infiltration of regulatory B cells. In some embodiments, the cancer is associated with infiltration of myeloid-derived suppressor cells. In some embodiments, the cancer is associated with infiltration of tumor-associated macrophages. In some embodiments, the cancer is associated with infiltration of innate lymphoid cells. In some embodiments, the cancer is associated with infiltration of cancer-associated fibroblasts. In some embodiments, the cancer is associated with a radiation-related increase in the above cell types.

In some embodiments, the cancer is associated with an increased TGFβ1 activation signature. In some embodiments the cancer is associated with an EMT or an EMT signature. In some embodiments the cancer is associated with a tumor exhibiting an EMT or an EMT signature and immune infiltration. In some embodiments the cancer is associated with a tumor profile of immune exclusion. In some embodiments, the cancer is associated with increased LAP expression. In some embodiments, the cancer is associated with increased GARP expression. In some embodiments, the cancer is associated with increased LRRC33 expression.

Cancers whose growth may be inhibited using the anti-LAP antibodies described herein include, but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer (e.g. estrogen-receptor positive breast cancer HER2-positive breast cancer; triple negative breast cancer); cancer of the peritoneum; cervical cancer; cholangiocarcinoma; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; liver cancer (e.g. hepatocellular carcinoma; hepatoma); intra-epithelial neoplasm; kidney or renal cancer; larynx cancer; leukemia; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; teratocarcinoma; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblasts leukemia; and post-transplant lymphoproliferative disorder (PTLD), as well as abnormal vascular proliferation associated with phakomatoses, edema (such as that associated with brain tumors), tumors of primitive origins and Meigs' syndrome.

Additional cancers which can be treated using the anti-LAP antibodies or antigen binding fragments thereof described herein include metastatic pancreatic cancer, metastatic adenocarcinoma of the pancreas, stomach cancer, fibrotic cancer, glioma, malignant glioma, diffuse intrinsic pontine glioma, recurrent childhood brain neoplasm renal cell carcinoma, clear-cell metastatic renal cell carcinoma, metastatic castration resistant prostate cancer, stage IV prostate cancer, metastatic melanoma, malignant melanoma, recurrent melanoma of the skin, melanoma brain metastases, malignant melanoma of head and neck, squamous cell non-small cell lung cancer, metastatic breast cancer, follicular lymphoma, advanced B-cell NHL, HL including diffuse large B-cell lymphoma (DLBCL), multiple myeloma, chronic myeloid leukemia, adult acute myeloid leukemia in remission, adult acute myeloid leukemia with Inv(16)(p13.1q22), CBFB-MYH11, adult acute myeloid leukemia with t(16:16) (p13.1:q22), CBFB-MYH11, adult acute myeloid leukemia with t(8:21)(d22:q22), RUNX1-RUNX1T1, adult acute myeloid leukemia with t(9:11)(p22:q23), MLLT3-MLL, adult acute promyelocytic leukemia with tO15:17)(q22:q12), PML-RARA, alkylating agent-related acute myeloid leukemia, Richter's syndrome, adult glioblastoma, adult gliosarcoma, recurrent glioblastoma, recurrent childhood rhabdomyosarcoma, recurrent ewing sarcoma/peripheral primitive neuroectodermal tumor, recurrent neuroblastoma, recurrent osteosarcoma, colorectal cancer, MSI positive colorectal cancer, MSI negative colorectal cancer, nasopharyngeal nonkeratinizing carcinoma, recurrent nasopharyngeal undifferentiated carcinoma, cervical adenocarcinoma, cervical adenosquamous carcinoma; cervical squamous cell carcinoma, recurrent cervical carcinoma, anal canal squamous cell carcinoma, metastatic anal canal carcinoma, recurrent anal canal carcinoma, recurrent head and neck cancer, squamous cell of head and neck, head and neck squamous cell carcinoma (HNSCC), ovarian carcinoma, colon cancer, advanced GI cancer, gastric adenocarcinoma, gastroesophageal junction adenocarcinoma, bone neoplasms, soft tissue sarcoma, bone sarcoma, thymic carcinoma, urothelial carcinoma, merkel cell carcinoma, recurrent merkel cell carcinoma, mycosis fungoides, Sezary syndrome, neuroendocrine cancer, nasopharyngeal cancer, basal cell skin cancer, squamous cell skin cancer, dermatofibrosarcoma trotuberans, glioma, mesothelioma, myelodysplastic syndromes (MDS), myelofibrosis (MF), myeloproliferative neoplasms, and acute myeloid leukemia (AMVL). Cancers may be metastatic or may be primary cancers. Cancers may be desmoplastic or non-desmoplastic. Cancers may be recurrent cancers.

In some embodiments, the anti-LAP antibodies or antigen binding fragments thereof described herein are used to treat myelodysplastic syndromes (MDS). MDS are a diverse group of malignant disorders marked by bone marrow failure due to defective hematopoiesis and production of dysplastic cells. TGFβ is a primary driver in MDS (Geyh et al., Haematologica 2018; 103:1462-71) and agents that inhibit the function of TGFβ have been proposed as therapeutics (Mies et al., Curr Hematol Malig Rep 2016; 11:416-24). Furthermore, MDSCs are known to be dysregulated in MDS (Chen et al., JCI 2013; 123:4595-611) and agents that reduce MDSC levels in the bone marrow are potential therapeutics.

In some embodiments, the anti-LAP antibodies or antigen binding fragments thereof described herein are used to treat myelofibrosis, which is another myeloid malignancy in which TGFβ1 plays a central role (Mascarenhas et al., Leukemia & Lymphoma 2014; 55:450-2).

In some embodiments, the cancer is resistant to checkpoint inhibitor(s). In some embodiments, the cancer is intrinsically refractory or resistant (e.g., resistant to a PD-1 pathway inhibitor, PD-1 pathway inhibitor, or CTLA-4 pathway inhibitor). In some embodiments, the resistance or refractory state of the cancer is acquired. In some embodiments, the anti-LAP antibodies or antigen binding fragments thereof described herein can be used in combination with checkpoint inhibitors to overcome resistance of the cancer to the checkpoint inhibitors. In some embodiments, the anti-LAP antibodies or antigen binding fragments thereof described herein can be used to treat tumors with a mesenchymal and/or EMT signature together with checkpoint inhibitors in combination or sequentially with agents that induce a mesenchymal phenotype, such as MAPK pathway inhibitors.

In some embodiments, the anti-LAP antibodies or antigen binding fragments thereof described herein are used to enhance the viability of immune cells ex vivo, e.g., in adoptive NK cell transfer. Accordingly, in some embodiments, anti-LAP antibodies are used in combination with adoptively transferred NK cells to treat cancer.

In some embodiments, the anti-LAP antibodies or antigen binding fragments thereof described herein are used to treat tumors with MHC loss or MHC down-regulation, as monotherapy or in combination with NK activating or enhancing treatment. In some embodiments, the anti-LAP antibodies described herein are used to treat checkpoint inhibitor resistant tumors in combination with NK activating or enhancing treatment.

Also provided herein is a method of treating cancer associated with an increased number of circulating platelets or an increased platelet to lymphocyte ratio comprising administering to a subject in need thereof an effective amount of an antibody or antigen binding fragment thereof described herein which specifically binds to LAP, wherein the antibody binds to platelets but does not cause platelet aggregation or platelet degranulation.

The ability of a compound to inhibit cancer can be evaluated in an animal model system predictive of efficacy in human tumors. Alternatively, this property of a composition can be evaluated by examining the ability of the compound to inhibit using in vitro assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

Also encompassed are methods for detecting the presence of LAP-TGFβ1 in a sample (e.g., a tumor biopsy sample), or measuring the amount of LAP-TGFβ1 in sample, comprising contacting the sample (e.g., tumor tissue) and a control sample (e.g., corresponding healthy tissue) with an antibody (e.g., monoclonal antibody) or antigen binding fragment thereof described herein which specifically binds to LAP-TGFβ1 under conditions that allow for formation of a complex between the antibody or portion thereof and LAP-TGFβ1. The formation of a complex is then detected, wherein a difference in complex formation between the sample compared to the control sample is indicative of the presence of LAP-TGFβ1 in the sample. The anti-LAP antibodies or antigen binding fragments thereof described herein can also be used to purify LAP-TGFβ1 via immunoaffinity purification.

Diagnostic applications of the anti-LAP antibodies described herein are also contemplated.

In one embodiment, provided herein is a method of diagnosing a cancer associated with regulatory T cell infiltration comprising contacting a biological sample from a patient afflicted with the cancer with an anti-LAP antibody or antigen binding fragment thereof described herein which binds to regulatory T cells, wherein positive staining with the antibody indicates the cancer is associated with regulatory T cell infiltration.

In another embodiment, provided herein is a method of diagnosing a cancer associated with GARP-negative suppressive cells comprising contacting a biological sample from a patient afflicted with the cancer with an anti-LAP antibody or antigen binding fragment thereof described herein which binds to GARP-negative suppressive cells, wherein positive staining with the antibody and negative staining with an anti-GARP antibody indicates the cancer is associated with GARP-negative suppressive cells.

In another embodiment, provided herein is a method of selecting a patient afflicted with cancer for treatment with an anti-LAP antibody or antigen binding fragment thereof described herein, comprising contacting a biological sample from the patient with the antibody, wherein positive staining with the antibody indicates the cancer is amenable to treatment with the antibody.

In another embodiment, provided herein is a method of determining the response of a patient afflicted with cancer to treatment with an anti-LAP antibody or antigen binding fragment thereof described herein comprising contacting a biological sample from the patient with the antibody, wherein reduced staining with the antibody indicates the cancer is responding to treatment with the antibody.

In another embodiment, provided herein is a method of determining whether a cancer in a patient has metastasized comprising (a) identifying a patient having a cancer, (b) administering a labeled (e.g., radiolabeled) anti-LAP antibody or antigen binding fragment thereof described herein to the patient and determining the biodistribution of the labeled anti-LAP antibody, and (c) periodically repeating step (b) to determine whether the biodistribution of the labeled anti-LAP antibody has changed, wherein a change in the biodistribution of the labeled anti-LAP antibody is indicative that the cancer has metastasized.

Also provided are methods of treating fibrosis with the anti-LAP antibodies described herein. In one embodiment, provided herein is a method of treating fibrosis comprising administering to a subject in need thereof an effective amount of an antibody or antigen binding fragment thereof described herein. In some embodiments, the fibrosis is associated with cancer. In some embodiments, the fibrosis is associated with increased levels of myeloid-derived suppressor cells (e.g. Fernandez et al., Eur Respir J 2016; 48:1171-83).

Exemplary fibrotic disorders which can be treated with any of the anti-LAP antibodies or antigen binding fragment thereof described herein include, but are not limited to, heart fibrosis, muscle fibrosis, skin fibrosis, liver fibrosis, soft tissue (e.g., mediastinum or retroperitoneum) fibrosis, renal fibrosis, bone marrow fibrosis, intestinal fibrosis, joint (e.g., knee, shoulder or other joints) fibrosis, pulmonary fibrosis, idiopathic pulmonary fibrosis, cystic fibrosis, endomyocardial fibrosis, mediastinal fibrosis, myelofibrosis, retroperitoneal fibrosis, progressive massive fibrosis, pipestem fibrosis, nephrogenic systemic fibrosis, Crohn's disease, keloid, old myocardial infarction, scleroderma/systemic sclerosis, subepithelial fibrosis, arthrofibrosis, some forms of adhesive capsulitis, proliferative fibrosis, viral hepatitis induced fibrosis, drug-induced fibrosis, radiation-induced fibrosis, and fibrosis associated with cancer.

Also provided herein is a method of reducing the number of immunosuppressive cells in a patient before, during, or after transplantation comprising administering an effective amount of any of the anti-LAP antibodies or antigen binding fragments thereof described herein to a patient before undergoing transplantation, during transplantation, and/or after transplantation. In some embodiments, the anti-LAP antibodies or antigen binding fragments thereof improve graft survival.

Inhibition of TGFβ has been shown to restore regenerative failure by reducing senescence and enhancing liver regeneration, in a model of acute liver disease (acetaminophen injury mouse model) (Bird et al., Sci Transl Med 2018; 10:eaan1230). Accordingly, also provided herein is a method of increasing the regenerative response in acute organ injury (e.g., acute liver injury) comprising administering to a subject with acute organ injury an effective amount of the anti-LAP antibodies or antigen binding fragments thereof described herein.

Aberrant activation of TGFβ has been shown to initiate the onset of temporomandibular joint osteoarthritis (Zheng et al., Bone Res 2018; 6:26). Accordingly, also provided herein is a method of treating a patient with temporomandibular joint osteoarthritis comprising administering to the patient an effective amount of the anti-LAP antibodies or antigen binding fragments thereof described herein to treat the temporomandibular joint osteoarthritis.

LAP-TGFβ1 has also been shown to mediate the differentiation of CD4+ effector cells into productively and latently infected central memory T cells during HIV-1 infection (Cheung et al., J Viol 2018; 92:e01510-17). Accordingly, also provided herein is a method of treating a patient with HIV-1 infection (or a patient at risk of developing HIV-1 infection) comprising administering to the patient an effective amount of the anti-LAP antibodies or antigen binding fragments thereof described herein to treat the HIV-1 infection (e.g., inhibit differentiation of CD4+ effector cells into productively and latently infected central memory T cells).

TGFβ-expressing macrophages and suppressive regulatory T cells have been shown to be altered in the peritoneal fluid of patients with endometriosis (Hanada et al., Reprod Biol Endocrinol 2018; 16:9), suggesting that targeting LAP-TGFb1 expressed on these cells may be beneficial for treating the disorder. Accordingly, also provided herein is a method of treating a patient with endometriosis comprising administering to the patient an effective amount of the anti-LAP antibodies or antigen binding fragments thereof described herein to treat the endometriosis.

LAP-TGFβ1-expressing CD4+ T cells and CD14+ monocytes and macrophages have been shown to be increased in patients carrying multidrug resistant Mycobacterium tuberculosis (Basile et al., Clin Exp Immunol 2016; 187:160), suggesting that targeting LAP-TGFβ1 expressed on these cells may be beneficial for treating the infection. Accordingly, also provided herein is a method of treating a patient with multidrug resistant Mycobacterium tuberculosis comprising administering to the patient an effective amount of the anti-LAP antibodies or antigen binding fragments thereof described herein (e.g., anti-LAP antibodies which inhibit LAP-TGFβ1 activation) to treat the infection.

In some embodiments, the anti-LAP antibodies or antigen binding fragments thereof described herein are used to treat p-thalassemia, a disorder in which TGFβ superfamily members have been implicated in defective erythropoiesis (Dussiot et al. Nat Med 2014; 20:398-407).

In certain embodiments, the anti-LAP antibody or antigen binding fragment can be used as monotherapy to treat a disease or disorder (e.g., cancer). Alternatively, an anti-LAP antibody or antigen binding fragment thereof can be used in conjunction with another agent or therapy, e.g., an anti-cancer agent, a chemotherapeutic agent, an immunosuppressive agent, an immunostimulatory agent, an immune checkpoint inhibitor, an anti-inflammatory agent, or a cell therapy, as described in more detail below.

### Combination Therapy

The anti-LAP antibodies or antigen binding fragments thereof described herein can be used in combination with various treatments or agents (or in the context of a multispecific antibody or bifunctional partner) known in the art for the treatment of cancer, as described herein.

Suitable anti-cancer agents for use in combination therapy with the anti-LAP antibodies or antigen binding fragments thereof described herein include, but are not limited to, surgery, chemotherapeutic agents, growth inhibitory agents, cytotoxic agents, radiotherapy and agents used in radiation therapy, anti-angiogenesis agents, apoptotic agents, anti-tubulin agents, and other agents to treat cancer, such as anti-HER-2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC (Imatinib Mesylate)), a COX-2 inhibitor (e.g., celecoxib), interferons, cytokines, antagonists (e.g., neutralizing antibodies) that bind to one or more of the following targets PD 1, PDL1, PDL2 (e.g., pembrolizumab; nivolumab; MK-3475; AMP-224; MPDL3280A; MEDIO680; MSB0010718C; and/or MEDI4736); CTLA4 (e.g., tremelimumab (PFIZER) and ipilimumab); LAG3 (e.g., BMS-986016); CD 103; TIM-3 and/or other TIM family members; CEACAM-1 and/or other CEACAM family members, ErbB2, ErbB3, ErbB4, PDGFR-beta, BlyS, APRIL, BCMA or VEGF receptor(s), TRAIL/Apo2, PARP inhibitors (e.g., AZD-2281, Lynparza Olaparib, Rubraca Rucaparib; (Zejula) niraparib), DNA damage repair inhibitors (e.g., ATMi, ATRi, DNAPKi), and other bioactive and organic chemical agents.

Combinations thereof are also specifically contemplated for the methods described herein. Suitable chemotherapeutic agents for use in combination therapy with the anti-LAP antibodies or antigen binding fragments thereof described herein include, but are not limited to, alkylating agents such as thiotepa and CYTOXAN® cyclosphosphamide; temozolomide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegal 1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, 111.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin, vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above.

Also suitable for use in combination with the anti-LAP antibodies or antigen binding fragments thereof described herein are drugs targeting epigenetic regulators, such as HDAC inhibitors, bromodomain inhibitors, and E3 ligase (e.g., cereblon) inhibitors (e.g., lenalidomide, pomalidomide, and thalidomide).

Suitable anti-inflammatory agents for use in combination therapy with the anti-LAP antibodies or antigen binding fragments thereof described herein include, but are not limited to, aspirin and other salicylates, Cox-2 inhibitors (e.g., rofecoxib and celecoxib), NSAIDs (such as ibuprofen, fenoprofen, naproxen, sulindac, diclofenac, piroxicam, ketoprofen, diflunisal, nabumetone, etodolac, oxaprozin, and indomethacin), anti-IL6R antibodies, anti-IL8 antibodies, anti-IL15 antibodies, anti-TL15R antibodies, anti-CD4 antibodies, anti-CD11a antibodies (e.g., efalizumab), anti-alpha-4/beta-1 integrin (VLA4) antibodies (e.g., natalizumab), CTLA4-Ig for the treatment of inflammatory diseases, prednisolone, prednisone, disease modifying antirheumatic drugs (DMARDs) such as methotrexate, hydroxychloroquine, sulfasalazine, pyrimidine synthesis inhibitors (e.g., leflunomide), IL-1 receptor blocking agents (e.g., anakinra), TNF-α blocking agents (e.g., etanercept, infliximab, and adalimumab), and the like.

Suitable immunomodulatory agents (e.g., immunostimulatory and immunosuppressive agents) include, but are not limited to, cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α, antibodies that bind to p75 of the IL-2 receptor, antibodies that bind to MHC, CD2, CD3, CD4, CD7, CD28, B7, CD40, CD45, IFN-γ, TNF-α, IL-4, IL-5, IL-6R, IL-6, IGF, IGFR1, IL-7, IL-8, IL-10, CD11a, or CD58, or antibodies binding to their ligands, soluble IL-15R, IL-10, B7 molecules (B7-1, B7-2, variants thereof, and fragments thereof), ICOS, OX40, an inhibitor of a negative T cell regulator (such as an antibody against CTLA4), and the like.

Additional immunosuppressive agents include, for example, anti-TNF agents such as etanercept, adalimumab and infliximab, and steroids. Examples of specific natural and synthetic steroids include, for example: aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, paramethasone, prednisolone, prednisone, tixocortol, and triamcinolone.

Suitable immunostimulatory agents for use in combination therapy with the anti-LAP antibodies or antigen binding fragments thereof described herein include, for example, compounds capable of stimulating antigen presenting cells (APCs), such as dendritic cells (DCs) and macrophages. For example, suitable immunostimulatory agents are capable of stimulating APCs, so that the maturation process of the APCs is accelerated, the proliferation of APCs is increased, and/or the recruitment or release of co-stimulatory molecules (e.g., CD80, CD86, ICAM-1, MHC molecules and CCR7) and pro-inflammatory cytokines (e.g., IL-1β, IL-6, IL-12, IL-15, and IFN-γ) is upregulated. Suitable immunostimulatory agents are also capable of increasing T cell proliferation. Such immunostimulatory agents include, but are not be limited to, CD40 ligand; FLT 3 ligand; cytokines, such as IFN-α, IFN-β, IFN-7 and IL-2; colony-stimulating factors, such as G-CSF (granulocyte colony-stimulating factor) and GM-CSF (granulocyte-macrophage colony-stimulating factor); an anti-CTLA-4 antibody, anti-PD1 antibody, anti-41BB antibody, or anti-OX-40 antibody; LPS (endotoxin); ssRNA; dsRNA; Bacille Calmette-Guerin (BCG); Levamisole hydrochloride; and intravenous immune globulins. In one embodiment an immunostimulatory agent may be a Toll-like Receptor (TLR) agonist. For example the immunostimulatory agent may be a TLR3 agonist such as double-stranded inosine:cytosine polynucleotide (Poly I:C, for example available as Ampligen™ from Hemispherx Bipharma, PA, US or Poly IC:LC from Oncovir) or Poly A:U; a TLR4 agonist such as monophosphoryl lipid A (MPL) or RC-529 (for example as available from GSK, UK); a TLR5 agonist such as flagellin; a TLR7 or TLR8 agonist such as an imidazoquinoline TLR7 or TLR 8 agonist, for example imiquimod (e.g., Aldara™) or resiquimod and related imidazoquinoline agents (e.g., as available from 3M Corporation); or a TLR 9 agonist such as a deoxynucleotide with unmethylated CpG motifs (“CpGs”, e.g., as available from Coley Pharmaceutical). In another embodiment, the immunostimulatory molecule is a STING agonist. Such immunostimulatory agents may be administered simultaneously, separately or sequentially with the anti-LAP antibodies or antigen binding fragments thereof described herein.

Suitable immune checkpoint blockers include, but are not limited to, agents (e.g., antibodies) that bind to PD-1, PD-L1, PD-L2, LAG-3, CTLA4, TIGIT, ICOS, OX40, PVR, PVRIG, VISTA, and TIM3. Non-limiting examples of antibodies that bind to PD-1, PD-L1, and PD-L2 include pembrolizumab; nivolumab; MK-3475; MPDL32; MEDIO680; MEDI4736; AMP-224; and MSB0010718C.

In some embodiments, the anti-LAP antibody or antigen binding fragment thereof is administered with an agent that targets a stimulatory or inhibitory molecule that is a member of the immunoglobulin super family (IgSF). For example, the anti-LAP antibodies or antigen binding fragments thereof described herein, may be administered to a subject with an agent that targets a member of the IgSF family to increase an immune response. For example, an anti-LAP antibody or antigen binding fragment thereof may be administered with an agent that targets a member of the B7 family of membrane-bound ligands that includes B7-1, B7-2, B7-H1 (PD-L1), B7-DC (PD-L2), B7-H2 (ICOS-L), B7-H3, B7-H4, B7-H5 (VISTA), and B7-H6 or a co-stimulatory or co-inhibitory receptor binding specifically to a B7 family member.

An anti-LAP antibody or antigen binding fragment thereof described herein may also be administered with an agent that targets a member of the TNF and TNFR family of molecules (ligands or receptors), such as CD40 and CD40L, OX-40, OX-40L, CD70, CD27L, CD30, CD30L, 4-1BBL, CD137, TRAIL/Apo2-L, TRAILR1/DR4, TRAILR2/DR5, TRAILR3, TRAILR4, OPG, RANK, RANKL, TWEAKR/Fni4, TWEAK, BAFFR, EDAR, XEDAR, TACI, APRIL, BCMA, LTOR, LIGHT, DcR3, HVEM, VEGI/TL1A, TRAMP/DR3, EDA1, EDA2, TNFR1, Lymphotoxin α/TNFβ, TNFR2, TNFα, LTβR, Lymphotoxin α 1β2, FAS, FASL, RELT, DR6, TROY, and NGFR (see, e.g., Tansey (2009) Drug Discovery Today 00:1).

T cell responses can be stimulated by a combination of anti-LAP antibodies or antigen binding fragments thereof described herein and one or more of the following agents:
(1) An antagonist (inhibitor or blocking agent) of a protein that inhibits T cell activation (e.g., immune checkpoint inhibitors), such as CTLA-4, PD-1, PD-L1, PD-L2, and LAG-3, as described above, and any of the following proteins: TIM-3, Galectin 9, CEACAM-1, BTLA, CD69, Galectin-1, TIGIT, CD 113, CD155, GPR56, VISTA, B7-H3, B7-H4, 2B4, CD48, GARP, PD1H, LAIR1, TIM-1, and TIM-4; and/or
(2) An agonist of a protein that stimulates T cell activation, such as B7-1, B7-2, CD28, 4-1BB (CD137), 4-1BBL, GITR, ICOS, ICOS-L, OX40, OX40L, CD70, CD27, CD40, DR3 and CD28H.

Exemplary agents that modulate the above proteins and may be combined with the anti-LAP antibodies or antigen binding fragments thereof described herein for treating cancer, include: Yervoy™ (ipilimumab) or Tremelimumab (to CTLA-4), galiximab (to B7.1), BMS-936558 (to PD-1), MK-3475 (to PD-1), AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3), Ipilumumab (to CTLA-4).

Other molecules that can be combined with anti-LAP antibodies or antigen binding fragments thereof described herein for the treatment of cancer include antagonists of inhibitory receptors on NK cells or agonists of activating receptors on NK cells. For example, anti-LAP antibodies or antigen binding fragments thereof can be combined with antagonists of KIR (e.g., lirilumab).

T cell activation is also regulated by soluble cytokines, and anti-LAP antibodies may be administered to a subject, e.g., having cancer, with antagonists of cytokines that inhibit T cell activation or agonists of cytokines that stimulate T cell activation.

In certain embodiments, anti-LAP antibodies or antigen binding fragments thereof described herein can be used in combination with (i) antagonists (or inhibitors or blocking agents) of proteins of the IgSF family or B7 family or the TNF family that inhibit T cell activation or antagonists of cytokines that inhibit T cell activation (e.g., IL-6, IL-10, TGF-β, VEGF; “immunosuppressive cytokines”) and/or (ii) agonists of stimulatory receptors of the IgSF family, B7 family or the TNF family or of cytokines that stimulate T cell activation, for stimulating an immune response, e.g., for treating proliferative diseases, such as cancer.

Yet other agents for combination therapies include agents that inhibit or deplete macrophages or monocytes, including but not limited to CSF-1R antagonists such as CSF-1R antagonist antibodies including RG7155 (see PCT publication numbers WO11/70024, WO11/107553, WO11/131407, WO13/87699, WO13/119716, and WO13/132044) or FPA-008 (see PCT publication numbers WO11/140249; WO13169264; and WO14/036357).

Additional agents that may be combined with anti-LAP antibodies or antigen binding fragments thereof described herein include agents that enhance tumor antigen presentation, e.g., dendritic cell vaccines, GM-CSF secreting cellular vaccines, CpG oligonucleotides, and imiquimod, or therapies that enhance the immunogenicity of tumor cells (e.g., anthracyclines).

Another therapy that may be combined with anti-LAP antibodies or antigen binding fragments thereof described herein is a therapy that inhibits a metabolic enzyme such as indoleamine dioxygenase (IDO), tryptophan-2,3-dioxygenase, dioxygenase, arginase, or nitric oxide synthetase.

Another class of agents that may be used with anti-LAP antibodies or antigen binding fragments thereof described herein includes agents that inhibit the formation of adenosine or inhibit the adenosine A2A receptor, for example, anti-CD73 antibodies, anti-CD39 antibodies, and adenosine A2A/A2b inhibitors.

Other therapies that may be combined with anti-LAP antibodies or antigen binding fragments thereof described herein for treating cancer include therapies that reverse/prevent T cell anergy or exhaustion and therapies that trigger an innate immune activation and/or inflammation at a tumor site.

The anti-LAP antibodies or antigen binding fragments thereof described herein may be combined with a combinatorial approach that targets multiple elements of the immune pathway, such as one or more of the following: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking regulatory T cells or other immune suppressing cells; a therapy that stimulates positive immune regulation, e.g., with agonists that stimulate the CD-137 and/or GITR pathway and/or stimulate T cell effector function; a therapy that increases systemically the frequency of anti-tumor T cells; a therapy that depletes or inhibits regulatory T cells using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); cell therapy with adoptive T cell or NK cell transfer including genetically modified cells, e.g., cells modified by chimeric antigen receptors (CAR-T therapy); a therapy that inhibits a metabolic enzyme such as indoleamine dioxygenase (IDO), dioxygenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immunosuppressive or immunorepressive cytokines.

The anti-LAP antibodies or antigen binding fragments thereof described herein can be combined with proinflammatory cytokines, for example, IL-12 and IL-2. These cytokines can be modified to enhance half-life and tumor targeting.

The anti-LAP antibodies or antigen binding fragments thereof described herein can be combined with immune cell engagers such as NK cell engagers or T cell engagers.

The anti-LAP antibodies or antigen binding fragments thereof described herein can be combined with indoleamine dioxygenase (IDO) inhibitors, tryptophan-2,3-dioxygenase (TDO) inhibitors, and dual IDO/TDO inhibitors.

The anti-LAP antibodies or antigen binding fragments thereof described herein can be combined with kynurine inhibitors.

The anti-LAP antibodies or antigen binding fragments thereof described herein can be combined with CD47 and/or SIRPa blocking therapies.

The anti-LAP antibodies or antigen binding fragments thereof described herein can be combined with JAK inhibitors and JAK pathway inhibitors (e.g., STAT3 inhibitors), e.g., for the treatment of myelofibrosis and myeloproliferative neoplasms.

The anti-LAP antibodies or antigen binding fragments thereof described herein can be combined with DNA damage repair inhibitors.

The anti-LAP antibodies or antigen binding fragments thereof described herein can be combined with erythropoietin and drugs that stimulate hematopoiesis.

The anti-LAP antibodies or antigen binding fragments thereof described herein can be combined with angiogenesis inhibitors.

The anti-LAP antibodies or antigen binding fragments thereof described herein can be combined with anti-viral drugs, such as neuramidase inhibitors.

Bispecific antibodies which have a first binding region with the specificity of the anti-LAP antibodies or antigen binding fragments thereof described herein and a second binding region which binds to an immune checkpoint blocker (e.g., PD-1, PD-L1) can be used in combination with at least one additional anti-cancer agent (e.g., radiation, chemotherapeutic agents, biologics, vaccines) to inhibit tumor growth.

The anti-LAP antibodies or antigen binding fragments thereof described herein can be combined with one or more immunostimulatory antibodies, such as an anti-PD-1 antagonist antibody, an anti-PD-L1 antagonist antibody, an antagonist anti-CTLA-4 antibody, an antagonistic anti-TIM3 antibody, and/or an anti-LAG3 antagonist antibody, such that an immune response is stimulated in the subject, for example to inhibit tumor growth.

Exemplary anti-PD-1 antibodies include nivolumab, pembrolizumab(also known as MK-3475,Lambrolizumab) described in WO2012/145493; AMP-514 described in WO 2012/145493, as well as PD-1 antibodies and other PD-1 inhibitors described in WO 2009/014708, WO 03/099196, WO 2009/114335, WO 2011/066389, WO 2011/161699, WO 2012/145493, U.S. Pat. Nos. 7,635,757 and 8,217,149, and U.S. Patent Publication No. 2009/0317368.

Exemplary anti-PD-L1 antibodies include MEDI4736 (also known as Anti-B7-H1), MPDL3280A (also known as RG7446), MSB0010718C (WO2013/79174), rHigM12B7, as well as any of the anti-PD-L1 antibodies disclosed in WO2013/173223, WO2011/066389, WO2012/145493, U.S. Pat. Nos. 7,635,757 and 8,217,149 and U.S. Publication No. 2009/145493.

Exemplary anti-CTLA-4 antibodies include Yervoy™ (ipilimumab), tremelimumab (formerly ticilimumab, CP-675,206), or an anti-CTLA-4 antibody described in any of the following publications: WO 98/42752; WO 00/37504; U.S. Pat. No. 6,207,156; Hurwitz et al. (1998) Proc. Natl. Acad. Sci. USA 95(17):10067-10071; Camacho et al. (2004)J. Clin. Oncology 22(145): Abstract No. 2505 (antibody CP-675206); and Mokyr et al. (1998) Cancer Res. 58:5301-5304.

Exemplary anti-LAG3 antibodies include IMP731 and IMP-321, described in US Publication No. 2011/007023, and PCT publication numbers WO08/132601, and WO09/44273, as well as antibodies described in U.S. Patent Publication No. US2011/0150892, and international patent publication numbers WO10/19570 and WO2014/008218.

Anti-LAP antibodies or antigen binding fragments thereof can also be combined with immune-oncology agents such as CD137 (4-1BB) agonists (e.g., an agonistic CD137 antibody such as urelumab or PF-05082566 (see PCT publication number WO12/32433)); GITR agonists (e.g., an agonistic anti-GITR antibody), CD40 agonists (e.g., an agonistic CD40 antibody); CD40 antagonists (e.g., an antagonistic CD40 antibody such as lucatumumab (HCD122), dacetuzumab (SGN-40), CP-870,893 or Chi Lob 7/4); CD27 agonists (e.g., an agonistic CD27 antibody such as varlilumab (CDX-1127)), MGA271 (to B7H3) (WO11/109400)); KIR antagonists (e.g., lirilumab); IDO antagonists (e.g., INCB-024360 (WO2006/122150, WO07/75598, WO08/36653, WO08/36642), indoximod, NLG-919 (WO09/73620, WO09/1156652, WO11/56652, WO12/142237) or F001287); Toll-like receptor agonists (e.g., TLR2/4 agonists (e.g., Bacillus Calmette-Guerin); TLR7 agonists (e.g., Hiltonol or Imiquimod); TLR7/8 agonists (e.g., Resiquimod); or TLR9 agonists (e.g., CpG7909)); and TGF-β inhibitors (e.g., GC1008, LY2157299, TEW7197, or IMC-TR1).

The anti-LAP antibodies or antigen binding fragments thereof described herein can also be combined with an immunogenic agent, such as cancerous cells, purified tumor antigens (including recombinant proteins, peptides, and carbohydrate molecules), cells, and cells transfected with genes encoding immune stimulating cytokines (He et al. (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF (discussed further below).

The anti-LAP antibodies or antigen binding fragments thereof described herein can also be combined with an anti-neoplastic antibody, such as Rituxan® (rituximab), Herceptin® (trastuzumab), Bexxar® (tositumomab), Zevalin® (ibritumomab), Campath® (alemtuzumab), Lymphocide® (eprtuzumab), Avastin® (bevacizumab), and Tarceva® (erlotinib), and the like.

Several experimental treatment protocols involve ex vivo activation and expansion of antigen specific T cells and adoptive transfer of these cells into recipients in order to antigen-specific T cells against tumor (Greenberg & Riddell, supra). Ex vivo activation in the presence of the anti-LAP antibodies described herein with or without an additional immunostimulating therapy (e.g., an immune checkpoint blocker) can be expected to increase the frequency and activity of the adoptively transferred T cells.

The anti-LAP antibody or antigen binding fragment thereof may also be administered with a standard of care treatment, or another treatment, such as radiation, surgery, or chemotherapy. The anti-LAP antibody or antigen binding fragment thereof may be combined with a vaccination protocol. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci U.S.A. 90: 3539-43). Dendritic cells (DC) are potent antigen presenting cells that can be used to prime antigen-specific responses. DC's can be produced ex vivo and loaded with various protein and peptide antigens as well as tumor cell extracts (Nestle et al. (1998) Nature Medicine 4: 328-332). DCs can also be transduced by genetic means to express these tumor antigens as well. DCs have also been fused directly to tumor cells for the purposes of immunization (Kugler et al. (2000) Nature Medicine 6:332-336). As a method of vaccination, DC immunization can be effectively combined with the anti-LAP antibodies or antigen binding fragments thereof described herein to activate more potent anti-tumor responses.

In some embodiments, the combination of therapeutic antibodies discussed herein can be administered concurrently as a single composition in a pharmaceutically acceptable carrier, or concurrently as separate compositions with each antibody in a pharmaceutically acceptable carrier. In another embodiment, the combination of therapeutic antibodies can be administered sequentially.

### X. Kits

Also provided are kits comprising the anti-LAP antibodies or antigen binding fragments thereof, multispecific molecules, or immunoconjugates disclosed herein, optionally contained in a single vial or container, and include, e.g., instructions for use in treating or diagnosing a disease (e.g., cancer). The kits may include a label indicating the intended use of the contents of the kit. The term label includes any writing, marketing materials or recorded material supplied on or with the kit, or which otherwise accompanies the kit. Such kits may comprise the antibody, multispecific molecule, or immunoconjugate in unit dosage form, such as in a single dose vial or a single dose pre-loaded syringe.

The present disclosure is further illustrated by the following examples, which should not be construed as further limiting. The contents of all figures and all references, Genbank sequences, patents, and published patent applications cited throughout this application are expressly incorporated herein by reference.

### EXAMPLES

Commercially available reagents referred to in the Examples below were used according to manufacturer's instructions unless otherwise indicated. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

### Example 1: Generation of LAP-TGFβ Target Proteins

Affinity-enhanced variants generated as described below were initially screened against fusion proteins consisting of residues 1-361 from human LAP-TGFβ1, a flexible linker containing a TEV protease site (GSTTENLYEQGSTG; SEQ ID NO: 93), and residues 118-447 (Eu numbering) from human IgG (SEQ ID NO: 1; Table 2). Similar constructs containing homologous residues from human LAP-TGFβ2, human LAP-TGFβ3, cynomolgus monkey LAP-TGFβ1, rat LAP-TGFβ31 and mouse LAP-TGFβ1 were also used to confirm binding specificity (Table 2). Some of these constructs used an alternative linker (GGGGSGGGGSGGGGS; SEQ ID NO: 94) and/or C4S/R249S to enhance stability. See, for example, SEQ ID NO: 5.

In Table 2 and subsequent Tables, unless indicated otherwise, it is understood that the CDRs in the binding protein (e.g., antibody or antigen binding fragment thereof) are identified by Kabat. Note that CDR(s) in a heavy chain variable region, light chain variable region, heavy chain, and/or light chain might be defined and identified by any of the methods and systems described herein (e.g., Chothia, Kabat, and IMGT in Table 1).

TABLE 2
LAP-TGFB-Fc fusion protein sequences

| SEQ | | |
|---|---|---|
| ID | | |
| NO. | Description | Amino acid sequence |
| 1 | human LAP-TGFB1 | MPPSGLRLLLLLLPLLWLLVLTPGRPAAGLSTCKTIDMELVKR |
| | Fc fusion | KRIEAIRGQILSKLRLASPPSQGEVPPGPLPEAVLALYNSTRD |
| | | RVAGESAEPEPEPEADYYAKEVTRVLMVETHNEIYDKFKQSTH |
| | | STYMFENTSELREAVPEPVLLSRAELRLLRLKLKVEQHVELYQ |
| | | KYSNNSWRYLSNRLLAPSDSPEWLSFDVTGVVRQWLSRGGEIE |
| | | GFRLSAHCSCDSRDNTLQVDINGFTTGRRGDLATIHGMNRPFL |
| | | LLMATPLERAQHLQSSRHRRALDTNYCFSSTEKNCCVRQLYID |
| | | FRKDLGWKWIHEPKGYHANFCLGPCPYIWSLDTQYSKVLALYN |
| | | QHNPGASAAPCCVPQALEPLPIVYYVGRKPKVEQLSNMIVRSC |
| | | KCSGSTTENLYFQGSTGTHTCPPCPAPELLGGPSVFLFPPKPK |
| | | DTLMISRTPEVTCVVVDVSHEDPEVKFNWYVDGVEVHNAKTKP |
| | | REEQYNSTYRVVSVLTVLHQDWLNGKEYKCKVSNKALPAPIEK |
| | | TISKAKGQPREPQVYTLPPSREEMTKNQVSLTCLVKGFYPSDI |
| | | AVEWESNGQPENNYKTTPPVLDSDGSFFLYSKLTVDKSRWQQG |
| | | NVFSCSVMHEALHNHYTQKSLSLSPGK |
| 2 | human LAP-TGFB2 | MHYCVLSAFLILHLVTVALSLSTSSTLDMDQFMRKRIEAIRGQ |
| | Fc fusion | ILSKLKLTSPPEDYPEPEEVPPEVISIYNSTRDLLQEKASRRA |
| | | AACERERSDEEYYAKEVYKIDMPPFFPSENAIPPTFYRPYFRI |
| | | VREDVSAMEKNASNLVKAEFRVFRLQNPKARVPEQRIELYQIL |
| | | KSKDLTSPTQRYIDSKVVKTRAEGEWLSFDVTDAVHEWLHHKD |
| | | RNLGFKISLHCPCCTFVPSNNYIIPNKSEELEARFAGIDGTST |
| | | YTSGDQKTIKSTRKKNSGKTPHLLLMLLPSYRLESQQTNRRKK |
| | | SALDAAYCFRNVQDNCCLRPLYIDFKRDLGWKWIHEPKGYNAN |
| | | FCAGACPYLWSSDTQHSRVLSLYNTINPEASASPCCVSQDLEP |
| | | LTILYYIGKTPKIEQLSNMIVKSCKCSGGGGSGGGGSGGGGSE |
| | | PKSCDKTHTCPPCPAPELLGGPSVFLFPPKPKDTLMISRTPEV |
| | | TCVVVDVSHEDPEVKFNWYVDGVEVHNAKTKPREEQYNSTYRV |
| | | VSVLTVLHQDWLNGKEYKCKVSNKALPAPIEKTISKAKGQPRE |
| | | PQVYTLPPSREEMTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPGK |
| 3 | human LAP-TGFB3 | MKMHLQRALVVLALLNFATVSLSLSTCTTLDFGHIKKKRVEAI |
| | Fc fusion | RGQILSKLRLTSPPEPTVMTHVPYQVLALYNSTRELLEEMHGE |
| | | REEGCTQENTESEYYAKEIHKFDMIQGLAEHNELAVCPKGITS |
| | | KVFRFNVSSVEKNRTNLFRAEFRVLRVPNPSSKRNEQRIELFQ |
| | | ILRPDEHIAKORYIGGKNLPTRGTAEWLSFDVTDTVREWLLRR |
| | | ESNLGLEISIHCPCHTFQPNGDILENIHEVMEIKFKGVDNEDD |
| | | HGRGDLGRLKKQKDHHNPHLILMMIPPHRLDNPGQGGQRKKSA |
| | | LDTNYCFRNLEENCCVRPLYIDFRQDLGWKWVHEPKGYYANFC |
| | | SGPCPYLRSADTTHSTVLGLYNTLNPEASASPCCVPQDLEPLT |
| | | ILYYVGRTPKVEQLSNMVVKSCKCSGGGGSGGGGSGGGGSEPK |
| | | SCDKTHTCPPCPAPELLGGPSVFLFPPKPKDTLMISRTPEVTC |
| | | VVVDVSHEDPEVKENWYVDGVEVHNAKTKPREEQYNSTYRVVS |
| | | VLTVLHQDWLNGKEYKCKVSNKALPAPIEKTISKAKGQPREPQ |
| | | VYTLPPSREEMTKNQVSLTCLVKGFYPSDIAVEWESNGQPENN |
| | | YKTTPPVLDSDGSFFLYSKLTVDKSRWQQGNVFSCSVMHEALH |
| | | NHYTQKSLSLSPGK |
| 4 | mouse LAP-TGFB1 | MPPSGLRLLPLLLPLPWLLVLTPGRPAAGLSTCKTIDMELVKR |
| | Fc fusion | KRIEAIRGQILSKLRLASPPSQGEVPPGPLPEAVLALYNSTRD |
| | | RVAGESADPEPEPEADYYAKEVTRVLMVDRNNAIYEKTKDISH |
| | | SIYMFFNTSDIREAVPEPPLLSRAELRLQRLKSSVEQHVELYQ |
| | | KYSNNSWRYLGNRLLTPTDTPEWLSFDVTGVVRQWLNQGDGIQ |
| | | GFRFSAHCSCDSKDNKLHVEINGISPKRRGDLGTIHDMNRPFL |
| | | LLMATPLERAQHLHSSRHRRALDTNYCFSSTEKNCCVRQLYID |
| | | FRKDLGWKWIHEPKGYHANFCLGPCPYIWSLDTQYSKVLALYN |
| | | QHNPGASASPCCVPQALEPLPIVYYVGRKPKVEQLSNMIVRSC |
| | | KCSGSTTENLYFQGSTGTHTCPPCPAPELLGGPSVFLFPPKPK |
| | | DTLMISRTPEVTCVVVDVSHEDPEVKFNWYVDGVEVHNAKTKP |
| | | REEQYNSTYRVVSVLTVLHQDWLNGKEYKCKVSNKALPAPIEK |
| | | TISKAKGQPREPQVYTLPPSREEMTKNQVSLTCLVKGFYPSDI |
| | | AVEWESNGQPENNYKTTPPVLDSDGSFFLYSKLTVDKSRWQQG |
| | | NVFSCSVMHEALHNHYTQKSLSLSPGK |
| 5 | rat LAP-TGFB1 | MPPSGLRLLLLLLPLLWLLVLTPGRPAAGLSTSKTIDMELVKR |
| | Fc fusion | KRIEAIRGQILSKLRLASPPSQGEVPPGPLPEAVLALYNSTRD |
| | | RVAGESADPEPEPEADYYAKEVTRVLMVDRNNAIYDKTKDITH |
| | | SIYMFFNTSDIREAVPEPPLLSRAELRLQRFKSTVEQHVELYQ |
| | | KYSNNSWRYLGNRLLTPTDTPEWLSFDVTGVVRQWLNQGDGIQ |
| | | GFRFSAHCSCDSKDNVLHVEINGISPKRRGDLGTIHDMNRPFL |
| | | LLMATPLERAQHLHSSRHRSALDTNYCFSSTEKNCCVRQLYID |
| | | FRKDLGWKWIHEPKGYHANFCLGPCPYIWSLDTQYSKVLALYN |
| | | QHNPGASASPCCVPQALEPLPIVYYVGRKPKVEQLSNMIVRSC |
| | | KCSGGGGSGGGGSGGGGSTHTCPPCPAPELLGGPSVFLFPPKP |
| | | KDTLMISRTPEVTCVVVDVSHEDPEVKENWYVDGVEVHNAKTK |
| | | PREEQYNSTYRVVSVLTVLHQDWLNGKEYKCKVSNKALPAPIE |
| | | KTISKAKGQPREPQVYTLPPSREEMTKNQVSLTCLVKGFYPSD |
| | | IAVEWESNGQPENNYKTTPPVLDSDGSFFLYSKLTVDKSRWQQ |
| | | GNVFSCSVMHEALHNHYTQKSLSLSPGK |
| 6 | cyno LAP-TGFB1 | MPPSGLRLLLLLLPLLWLLVLTPGRPAAGLSTSKTIDMELVKR |
| | | KRIEAIRGQILSKLRLASPPSQGEVPPGPLPEAVLALYNSTRD |
| | | RVAGESAEPEPEPEADYYAKEVTRVLMVETHNEIYDKFKQSTH |
| | | SIYMFFNTSELREAVPEPVLLSRAELRLLRLKLKVEQHVELYQ |
| | | KYSNNSWRYLSNRLLAPSDSPEWLSFDVTGVVRQWLSRGGEIE |
| | | GFRLSAHCSCDSKDNTLQVDINGFTTGRRGDLATIHGMNRPFL |
| | | LLMATPLERAQHLQSSRHRRALDTNYCFSSTEKNCCVRQLYID |
| | | FRKDLGWKWIHEPKGYHANFCLGPCPYIWSLDTQYSKVLALYN |
| | | QHNPGASAAPCCVPQALEPLPIVYYVGRKPKVEQLSNMIVRSC |
| | | KCSGSTTENLYFQGSTGTHTCPPCPAPELLGGPSVFLFPPKPK |
| | | DTLMISRTPEVTCVVVDVSHEDPEVKFNWYVDGVEVHNAKTKP |
| | | REEQYNSTYRVVSVLTVLHQDWLNGKEYKCKVSNKALPAPIEK |
| | | TISKAKGQPREPQVYTLPPSREEMTKNQVSLTCLVKGFYPSDI |
| | | AVEWESNGQPENNYKTTPPVLDSDGSFFLYSKLTVDKSRWQQG |
| | | NVFSCSVMHEALHNHYTQKSLSLSPGK |

Codon-optimized genes for the amino-acid sequences described in Table 1 were subcloned into pcDNA3.4 for protein expression. These plasmids were transfected into ExpiCHO cells to produce secreted fusion protein in the culture medium. The host cells were removed by centrifugation (4,000×g) for 10 minutes, followed by filtration through a 0.22-micron sterile filter. Fusion proteins were isolated from filtered media by Protein A chromatography. Preparative size-exclusion chromatography was used to isolate monomeric LAP-TGFβ-Fc fusion protein (˜140 kDa).

To control for possible off-target binding to the human IgG1 Fc portion of the fusion protein, a human LAP-TGFβ1 protein with N-terminal polyhistidine and StrepTagII tags was produced (Table 3). A codon-optimized gene for this protein was subcloned into pcDNA3.4 for protein expression. The expression plasmid was transfected into ExpiCHO cells to produce secreted LAP-TGFβ1 in the culture medium. The LAP-TGFβ1 protein was purified by a sequence of NiNTA affinity chromatograph, cation-exchange chromatography and size-exclusion chromatography.

TABLE 3
His8-StrepTagII-TEV-LAP-TGFB1 sequence

| SEQ | Descrip- | |
|---|---|---|
| ID | tion | Sequence |
| 7 | human | MPPSGLRLLLLLLPLLWLLV |
| | LAP-TGFB1 | LTPGRPAAGHHHHHHHHGGG |
| | construct | SWSHPQFEKGGGSGGGSGGS |
| | with | SAWSHPQFEKENLYFQGLST |
| | leader | SKTIDMELVKRKRIEAIRGQ |
| | sequence | ILSKLRLASPPSQGEVPPGP |
| | | LPEAVLALYNSTRDRVAGES |
| | | AEPEPEPEADYYAKEVTRVL |
| | | MVETHNEIYDKFKQSTHSIY |
| | | MFFNTSELREAVPEPVLLSR |
| | | AELRLLRLKLKVEQHVELYQ |
| | | KYSNNSWRYLSNRLLAPSDS |
| | | PEWLSFDVTGVVRQWLSRGG |
| | | EIEGFRLSAHCSCDSRDNTL |
| | | QVDINGFTTGRRGDLATIHG |
| | | MNRPFLLLMATPLERAQHLQ |
| | | SSRHRSALDTNYCFSSTEKN |
| | | CCVRQLYIDERKDLGWKWIH |
| | | EPKGYHANFCLGPCPYIWSL |
| | | DTQYSKVLALYNQHNPGASA |
| | | APCCVPQALEPLPIVYYVGR |
| | | KPKVEQLSNMIVRSCKCS |

### Example 2. Production of 20E6 Variants Targeting LAP/TGFβ1

The 20E6 antigen-binding fragment (Fab) sequences were cloned into heavy and light chain expression plasmids for protein production. Combinations of heavy and light chain expression plasmids were co-transfected into ExpiCHO cells to produce secreted antibody in the culture medium. Various methods were used to produce Fab proteins at different scales. At a smaller scale, for screening purposes, the volume of culture was 5-mL and produced within a 24-deep well plate at 37° C., 250-RPM, 8% C02, and 80% relative humidity for the first 24-hours of expression, followed by 32° C., 250-RPM, 8% C02, and 80% relative humidity for the remainder of the expression period. At larger scales of expression, the total volume of culture ranged from 30-mL to 250-mL in Corning® shake flasks, non-baffled. The conditions of expressions were 37° C., 250-RPM, 8% CO2, and 80% relative humidity for the first 24-hours of expression, followed by 32° C., 125-RPM, 8% CO2, and 80% relative humidity for the remainder of the expression period.

Fab proteins were purified by KappaSelect affinity chromatography. Proteins were eluted using 20 mM sodium acetate (pH 3.0) and neutralized using 1 M tris(hydroxymethyl)aminomethane (TRIS) buffer at pH 8.0. Size-exclusion chromatography was used to characterize the homogeneity of the eluted antibody and concentration was measured using absorbance at 280 nm (A₂₈₀) with a calculated extinction co-efficient.

### Example 3: Molecular Interaction Analysis by ForteBio Interferometry

Anti-human Fab-CH1 2^{nd} generation (FAB2G, catalog 18-5125) biosensors were prepared for loading of purified Fab by soaking for 10 minutes in assay buffer, 1× phosphate buffered saline (PBS) 0.1% bovine serum albumin, BSA (10× PBS—Gibco, catalog 70013032, BSA—Jackson Immunoresearch, catalog 001-000-162). The Fab mutants including a positive control, 20E6 Fab, and a negative control Fab (Molecular Innovations, catalog HU-FAB-4510) were diluted to 200 nM in assay buffer and loaded to FAB2G sensors for 180 seconds followed by a one-hour incubation at room temperature in assay buffer to equilibrate the tips. The Fab loaded sensors were assessed for binding with 100 nM human LAP-TGFβ for 180 seconds followed by a 180 second dissociation phase in assay buffer on a ForteBio HTX (software version 9.0.0.66). Non-specific binding to the negative control Fab was subtracted and the binding sensorgrams were aligned to the start of the association phase on the X and Y axis. The Fab binding was fit with the 1:1 binding model, dissociation phase only, with local fitting using ForteBio Data Analysis 9.0 software.

### Example 4: Engineering Computationally Designed Variants

Further studies were performed with the 20E6 antibody and antigen-binding fragments thereof such that the molecules were engineered to include modifications to CDR residues within the variable domains of the humanized monoclonal antibody, e.g., to improve a property of the antibody or antigen binding fragment thereof. The modifications were made to increase the affinity of the antibody or antigen binding fragment thereof. Affinity enhancement using directed evolution methods have been shown to be effective for this purpose. The structure and interaction of 20E6 antibody and the LAP/TGF-beta structure were determined. Investigators then sought to investigate the applicability of computational methods to enhance affinity. Computational design methods have the ability to enhance antibody-antigen affinity. A variety of tools and methods for designing variants were implemented.

In order to generate a diverse set of computational designs to test experimentally, investigators utilized multiple computational approaches in parallel to design affinity enhancing mutants. These methods included: saturation mutagenesis (exhaustively sampling all possible single point mutations within the antigen binding region and the associated scoring of all resulting antibody-antigen complexes) through the independent application of molecular operating environment (MOE) modeling (https://www.schrodinger.com/BioLuminate/) and Bioluminate modeling by Schrodinger software, stochastic sequence sampling in MOE (extensively sampling single, double, and triple mutant combinations within the antigen binding region and the associated scoring of these sampled constructs), and multi-state modeling (Rosetta Design) to allow for greater flexibility in antigen binding during antibody mutant exploration than other methods would allow. In addition, visual structure-based modeling and design was used to generate an independent list of mutants that were added to the final list of constructs. Designs that scored highly by multiple methods which also passed visual inspection were prioritized and included in experimental characterization (Table 4).

TABLE 4
20E6 variants containing computationally designed
substitutions in selected CDR positions

| Seq | | |
|---|---|---|
| ID | | |
| NO. | Identifier | Amino acid sequence |
| 8 | VL R53D (35BIT) LCDR1 | RASQDITNYLN |
| 9 | VL R53D (35BIT) LCDR2 | YTSDLHS |
| 10 | VL R53D (35BIT) LCDR3 | QQGDTLPWT |
| 11 | VL R53D (35BIT) VL | DIQMTQSPSSLSASVGDRVTITCRASQDITNYLNWYQQKPGK |
| | | AVKLLIYYTSDLHSGVPSRFSGSGSGTDYTLTISSLQPEDFA |
| | | TYFCQQGDTLPWTFGQGTKLEIK |
| 12 | VL R53D (35BIT) LC | DIQMTQSPSSLSASVGDRVTITCRASQDITNYLNWYQQKPGK |
| | | AVKLLIYYTSDLHSGVPSRFSGSGSGTDYTLTISSLQPEDFA |
| | | TYFCQQGDTLPWTFGQGTKLEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSEN |
| | | RGEC |
| 13 | VL R53I (34BIT) LCDR1 | RASQDITNYLN |
| 14 | VL R53I (34BIT) LCDR2 | YTSILHS |
| 15 | VL R53I (34BIT) LCDR3 | QQGDTLPWT |
| 16 | VL R53I (34BIT) VL | DIQMTQSPSSLSASVGDRVTITCRASQDITNYLNWYQQKPGK |
| | | AVKLLIYYTSILHSGVPSRFSGSGSGTDYTLTISSLQPEDFA |
| | | TYFCQQGDTLPWTFGQGTKLEIK |
| 17 | VL R53I (34BIT) LC | DIQMTQSPSSLSASVGDRVTITCRASQDITNYLNWYQQKPGK |
| | | AVKLLIYYTSILHSGVPSRFSGSGSGTDYTLTISSLQPEDFA |
| | | TYFCQQGDTLPWTFGQGTKLEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| 18 | VL R53E (31BIT) LCDR1 | RASQDITNYLN |
| 19 | VL R53E (31BIT) LCDR2 | YTSELHS |
| 20 | VL R53E (31BIT) LCDR3 | QQGDTLPWT |
| 21 | VL R53E (31BIT) VL | DIQMTQSPSSLSASVGDRVTITCRASQDITNYLNWYQQKPGK |
| | | AVKLLIYYTSELHSGVPSRESGSGSGTDYTLTISSLQPEDFA |
| | | TYFCQQGDTLPWTFGQGTKLEIK |
| 22 | VL R53E (31BIT) LC | DIQMTQSPSSLSASVGDRVTITCRASQDITNYLNWYQQKPGK |
| | | AVKLLIYYTSELHSGVPSRFSGSGSGTDYTLTISSLQPEDFA |
| | | TYFCQQGDTLPWTFGQGTKLEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSEN |
| | | RGEC |
| 23 | VL T30E (36BIT) LCDR1 | RASQDIENYLN |
| 24 | VL T30E (36BIT) LCDR2 | YTSRLHS |
| 25 | VL T30E (36BIT) LCDR3 | QQGDTLPWT |
| 26 | VL T30E (36BIT) VL | |
| | | TYFCQQGDTLPWTFGQGTKLEIK |
| 27 | VL T30E (36BIT) LC | DIQMTQSPSSLSASVGDRVTITCRASQDIENYLNWYQQKPGK |
| | | AVKLLIYYTSRLHSGVPSRFSGSGSGTDYTLTISSLQPEDFA |
| | | TYFCQQGDTLPWTFGQGTKLEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| 28 | VL T30D (37BIT) LCDR1 | RASQDIDNYLN |
| 29 | VL T30D (37BIT) LCDR2 | YTSRLHS |
| 30 | VL T30D (37BIT) LCDR3 | QQGDTLPWT |
| 31 | VL T30D (37BIT) VL | DIQMTQSPSSLSASVGDRVTITCRASQDIDNYLNWYQQKPGK |
| | | AVKLLIYYTSRLHSGVPSRFSGSGSGTDYTLTISSLQPEDFA |
| | | TYFCQQGDTLPWTFGQGTKLEIK |
| 32 | VL T30D (37BIT) LC | DIQMTQSPSSLSASVGDRVTITCRASQDIDNYLNWYQQKPGK |
| | | AVKLLIYYTSRLHSGVPSRESGSGSGTDYTLTISSLQPEDFA |
| | | TYFCQQGDTLPWTFGQGTKLEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| 33 | VL L94D (32BIT) LCDR1 | RASQDITNYLN |
| 34 | VL L94D (32BIT) LCDR2 | YTSRLHS |
| 35 | VL L94D (32BIT) LCDR3 | QQGDTDPWT |
| 36 | VL L94D (32BIT) VL | DIQMTQSPSSLSASVGDRVTITCRASQDITNYLNWYQQKPGK |
| | | AVKLLIYYTSRLHSGVPSRFSGSGSGTDYTLTISSLQPEDFA |
| | | TYFCQQGDTDPWTFGQGTKLEIK |
| 37 | VL L94D (32BIT) LC | DIQMTQSPSSLSASVGDRVTITCRASQDITNYLNWYQQKPGK |
| | | AVKLLIYYTSRLHSGVPSRESGSGSGTDYTLTISSLQPEDFA |
| | | TYFCQQGDTDPWTFGQGTKLEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| 38 | VL L94Q (33BIT) LCDR1 | RASQDITNYLN |
| 39 | VL L94Q (33BIT) LCDR2 | YTSRLHS |
| 40 | VL L94Q (33BIT) LCDR3 | QQGDTQPWT |
| 41 | VL L94Q (33BIT) VL | DIQMTQSPSSLSASVGDRVTITCRASQDITNYLNWYQQKPGK |
| | | AVKLLIYYTSRLHSGVPSRFSGSGSGTDYTLTISSLQPEDFA |
| | | TYFCQQGDTQPWTFGQGTKLEIK |
| 42 | VL L94Q (33BIT) LC | DIQMTQSPSSLSASVGDRVTITCRASQDITNYLNWYQQKPGK |
| | | AVKLLIYYTSRLHSGVPSRFSGSGSGTDYTLTISSLQPEDFA |
| | | TYFCQQGDTQPWTFGQGTKLEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| 43 | VH S31K (24BIT) HCDR1 | KYWMH |
| 44 | VH S31K (24BIT) HCDR2 | RIDPQSGGIKYAQKFQG |
| 45 | VH S31K (24BIT) HCDR3 | WDYGGYFDV |
| 46 | VH S31K (24BIT) VH | EVOLVQSGAEVKKPGASVKVSCKASGYTFTKYWMHWVRQAPG |
| | | QGLEWMGRIDPQSGGIKYAQKFQGRATLTVDTSTSTAYMELS |
| | | RLRSDDTAVYYCARWDYGGYFDVWGQGTLVTVSS |
| 47 | VH S31K (24BIT) Fab_HC | EVQLVQSGAEVKKPGASVKVSCKASGYTFTKYWMHWVRQAPG |
| | | QGLEWMGRIDPQSGGIKYAQKFQGRATLTVDTSTSTAYMELS |
| | | RLRSDDTAVYYCARWDYGGYFDVWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHT |
| 48 | VH S31R (25BIT) HCDR1 | RYWMH |
| 49 | VH S31R (25BIT) HCDR2 | RIDPQSGGIKYAQKFQG |
| 50 | VH S31R (25BIT) HCDR3 | WDYGGYFDV |
| 51 | VH S31R (25BIT) VH | EVQLVQSGAEVKKPGASVKVSCKASGYTFTRYWMHWVRQAPG |
| | | QGLEWMGRIDPQSGGIKYAQKFQGRATLTVDTSTSTAYMELS |
| | | RLRSDDTAVYYCARWDYGGYFDVWGQGTLVTVSS |
| 52 | VH S31R (25BIT) Fab_HC | EVQLVQSGAEVKKPGASVKVSCKASGYTFTRYWMHWVRQAPG |
| | | QGLEWMGRIDPQSGGIKYAQKFQGRATLTVDTSTSTAYMELS |
| | | RLRSDDTAVYYCARWDYGGYFDVWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHT |
| 53 | VH H35R (62BIT) HCDR1 | SYWMR |
| 54 | VH H35R (62BIT) HCDR2 | RIDPQSGGIKYAQKFQG |
| 55 | VH H35R (62BIT) HCDR3 | WDYGGYFDV |
| 56 | VH H35R (62BIT) VH | EVQLVQSGAEVKKPGASVKVSCKASGYTFTSYWMRWVRQAPG |
| | | QGLEWMGRIDPQSGGIKYAQKFQGRATLTVDTSTSTAYMELS |
| | | RLRSDDTAVYYCARWDYGGYFDVWGQGTLVTVSS |
| 57 | VH H35R (62BIT) Fab_HC | EVQLVQSGAEVKKPGASVKVSCKASGYTFTSYWMRWVRQAPG |
| | | QGLEWMGRIDPQSGGIKYAQKFQGRATLTVDTSTSTAYMELS |
| | | RLRSDDTAVYYCARWDYGGYFDVWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHT |
| 58 | VH H35K (30BIT) HCDR1 | SYWMK |
| 59 | VH H35K (30BIT) HCDR2 | RIDPQSGGIKYAQKFQG |
| 60 | VH H35K (30BIT) HCDR3 | WDYGGYFDV |
| 61 | VH H35K (30BIT) VH | EVQLVQSGAEVKKPGASVKVSCKASGYTFTSYWMKWVRQAPG |
| | | QGLEWMGRIDPQSGGIKYAQKFQGRATLTVDTSTSTAYMELS |
| | | RLRSDDTAVYYCARWDYGGYFDVWGQGTLVTVSS |
| 62 | VH H35K (30BIT) HC | EVQLVQSGAEVKKPGASVKVSCKASGYTFTSYWMKWVRQAPG |
| | | QGLEWMGRIDPQSGGIKYAQKFQGRATLTVDTSTSTAYMELS |
| | | RLRSDDTAVYYCARWDYGGYFDVWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHT |
| 63 | VH S55E (26BIT) HCDR1 | SYWMH |
| 64 | VH S55E (26BIT) HCDR2 | RIDPQEGGIKYAQKFQG |
| 65 | VH S55E (26BIT) HCDR3 | WDYGGYFDV |
| 66 | VH S55E (26BIT) VH | EVOLVQSGAEVKKPGASVKVSCKASGYTFTSYWMHWVRQAPG |
| | | QGLEWMGRIDPQEGGIKYAQKFQGRATLTVDTSTSTAYMELS |
| | | RLRSDDTAVYYCARWDYGGYFDVWGQGTLVTVSS |
| 67 | VH S55E (26BIT) Fab_HC | EVQLVQSGAEVKKPGASVKVSCKASGYTFTSYWMHWVRQAPG |
| | | QGLEWMGRIDPQEGGIKYAQKFQGRATLTVDTSTSTAYMELS |
| | | RLRSDDTAVYYCARWDYGGYFDVWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHT |
| 68 | VH G57E (63BIT) HCDR1 | SYWMH |
| 69 | VH G57E (63BIT) HCDR2 | RIDPQSGEIKYAQKFQG |
| 70 | VH G57E (63BIT) HCDR3 | WDYGGYFDV |
| 71 | VH G57E (63BIT) VH | EVQLVQSGAEVKKPGASVKVSCKASGYTFTSYWMHWVRQAPG |
| | | QGLEWMGRIDPQSGEIKYAQKFQGRATLTVDTSTSTAYMELS |
| | | RLRSDDTAVYYCARWDYGGYFDVWGQGTLVTVSS |
| 72 | VH G57E (63BIT) Fab_HC | EVQLVQSGAEVKKPGASVKVSCKASGYTFTSYWMHWVRQAPG |
| | | QGLEWMGRIDPQSGEIKYAQKFQGRATLTVDTSTSTAYMELS |
| | | RLRSDDTAVYYCARWDYGGYFDVWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHT |
| 73 | VH G57D (27BIT) HCDR1 | SYWMH |
| 74 | VH G57D (27BIT) HCDR2 | RIDPQSGDIKYAQKFQG |
| 75 | VH G57D (27BIT) HCDR3 | WDYGGYFDV |
| 76 | VH G57D (27BIT) VH | EVQLVQSGAEVKKPGASVKVSCKASGYTFTSYWMHWVRQAPG |
| | | QGLEWMGRIDPQSGDIKYAQKFQGRATLTVDTSTSTAYMELS |
| | | RLRSDDTAVYYCARWDYGGYFDVWGQGTLVTVSS |
| 77 | VH G57D (27BIT) Fab_HC | EVOLVQSGAEVKKPGASVKVSCKASGYTFTSYWMHWVRQAPG |
| | | QGLEWMGRIDPQSGDIKYAQKFQGRATLTVDTSTSTAYMELS |
| | | RLRSDDTAVYYCARWDYGGYFDVWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHT |
| 78 | VH K59W (61BIT) HCDR1 | SYWMH |
| 79 | VH K59W (61BIT) HCDR2 | RIDPQSGGIWYAQKFQG |
| 80 | VH K59W (61BIT) HCDR3 | WDYGGYFDV |
| 81 | VH K59W (61BIT) VH | EVOLVQSGAEVKKPGASVKVSCKASGYTFTSYWMHWVRQAPG |
| | | QGLEWMGRIDPQSGGIWYAQKFQGRATLTVDTSTSTAYMELS |
| | | RLRSDDTAVYYCARWDYGGYFDVWGQGTLVTVSS |
| 82 | VH K59W (61BIT) Fab_HC | EVQLVQSGAEVKKPGASVKVSCKASGYTFTSYWMHWVRQAPG |
| | | QGLEWMGRIDPQSGGIWYAQKFQGRATLTVDTSTSTAYMELS |
| | | RLRSDDTAVYYCARWDYGGYFDVWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHT |
| 83 | VH K59Q (28BIT) HCDR1. | SYWMH |
| 84 | VH K59Q (28BIT) HCDR2 | RIDPQSGGIQYAQKFQG |
| 85 | VH K59Q (28BIT) HCDR3 | WDYGGYFDV |
| 86 | VH K59Q (28BIT) VH | EVOLVQSGAEVKKPGASVKVSCKASGYTFTSYWMHWVRQAPG |
| | | QGLEWMGRIDPQSGGIQYAQKFQGRATLTVDTSTSTAYMELS |
| | | RLRSDDTAVYYCARWDYGGYFDVWGQGTLVTVSS |
| 87 | VH K59Q (28BIT) Fab_HC | EVQLVQSGAEVKKPGASVKVSCKASGYTFTSYWMHWVRQAPG |
| | | QGLEWMGRIDPQSGGIQYAQKFQGRATLTVDTSTSTAYMELS |
| | | RLRSDDTAVYYCARWDYGGYFDVWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHT |
| 88 | VH G102S (29BIT) HCDR1 | SYWMH |
| 89 | VH G102S (29BIT) HCDR2 | RIDPQSGGIKYAQKFQG |
| 90 | VH G102S (29BIT) HCDR3 | WDYSGYFDV |
| 91 | VH G102S (29BIT) VH | EVQLVQSGAEVKKPGASVKVSCKASGYTFTSYWMHWVRQAPG |
| | | QGLEWMGRIDPQSGGIKYAQKFQGRATLTVDTSTSTAYMELS |
| | | RLRSDDTAVYYCARWDYSGYFDVWGQGTLVTVSS |
| 92 | VH G102S (29BIT) | EVQLVQSGAEVKKPGASVKVSCKASGYTFTSYWMHWVRQAPG |
| | Fab_HC | QGLEWMGRIDPQSGGIKYAQKFQGRATLTVDTSTSTAYMELS |
| | | RLRSDDTAVYYCARWDYSGYFDVWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHT |

As shown in Table 5, eleven of the sixty-five in silico design mutants exhibited at least 1.5 improvement in off-rate as compared to the humanized 20E6 Fab controls. Fold improvement in off-rate was calculated by dividing the average off-rate for the combined parental controls (i.e., 4.68×10⁻² and 3.81×10⁻²) by the mutant Fab off-rate.

TABLE 5
ForteBio off-rate screen for in silico
designed Fab mutants with human TGFβ1

| | Binding | k_{off} | Fold |
|---|---|---|---|
| Identifier | response (nm) | (×10⁻² s⁻¹) | change |
| VL R53D (35BIT) | 0.398 | 3.41 | 1.2 |
| VL R531 (34BIT) | 0.757 | 2.46 | 1.7 |
| VL R53E (31BIT) | 0.664 | 2.86 | 1.5 |
| VL T30E (36BIT) | 0.439 | 4.05 | 1 |
| VL T30D (37BIT) | 0.182 | 4.02 | 1.1 |
| VL L94D (32BIT) | 0.195 | 6.18 | 0.7 |
| VL L94Q (33BIT) | 0.341 | 5.83 | 0.7 |
| VH S31K (24BIT) | 0.625 | 3.7 | 1.1 |
| VH S31R (25BIT) | 0.069 | 4.37 | 1 |
| VH H35R (62BIT) | 0.021 | NB | NA |
| VH H35K (30BIT) | 0.115 | 5.48 | 0.8 |
| VH S55E (26BIT) | 0.489 | 5.19 | 0.8 |
| VH G57E (63BIT) | 0.69 | 3.93 | 1.1 |
| VH G57D (27BIT) | 0.582 | 3.37 | 1.3 |
| VH K59W (61BIT) | 0.035 | NB | NA |
| VH K59Q (28BIT) | 0.165 | 5.96 | 0.7 |
| VH G102S (29BIT) | 0.087 | 8.45 | 0.5 |
| 20E6 (80BGJ) | 0.253 | 4.68 | 1 |
| 20E6 (85BIT) | 0.58 | 3.81 | 1.1 |

NB: No binding observed
NA: Not applicable

### Example 5: Tyrosine Scanning for Affinity-Improved 20E6 Variants by ForteBio Interferometry

The following studies analyzed whether CDR residue substitutions by aromatic amino acids such as tyrosine, phenylalanine and tryptophan could be used to improve off-rate and thus overall binding affinity of an antibody to its cognate target antigen. Of the three, tyrosine substitutions were preferred for its lower hydrophobicity and resistance to oxidation. Thus, investigators employed a ‘tyrosine scanning’ strategy to systemically replace individual CDR residues in 20E36 antibody and identify variants that had improved binding activity to human LAP-TGFb protein. This approach more efficient than exhaustively replacing each CDR residues by all available amino acids through gene synthesis or degenerate NNK or NNS codon substitution; and is contrary to the common ‘alanine scanning’ method that is often used to identify CDR residues that when replaced by alanine results in loss of affinity and thus are important for antigen binding. It is the investigators approach that the unbiased tyrosine scanning strategy can be performed independent of structural information and may identify surprising and unexpected CDR residues that may not be obvious choices by structural studies.

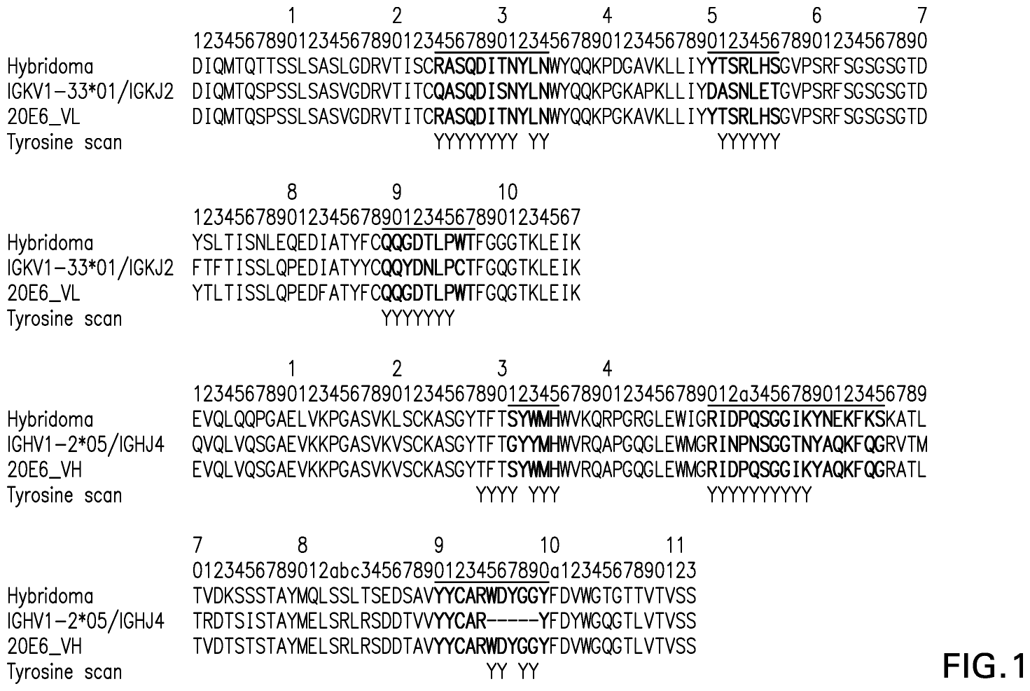

Twenty-three variants containing a single tyrosine substitution in the light chain CDRs (at one of amino acid positions 24-31, 33, 34, 51-56, and 89-95 according to Kabat numbering scheme) and 21 similar heavy chain CDR variants (at amino acids positions 28-31, 33-35, 50-58 including 52a, 95, 96, 98, and 99 according to Kabat numbering scheme) were generated according to FIG. 1 and listed in Table 6 and their interactions with human LAP-TGFb1 were analyzed by ForteBio interferometry. Please note that in numerous sequences in Table 6 there are sequences listing an identifier such as the VL_X ##Y (see for example SEQ IDE NOs: 95-99 m and 105-109), which refers to the mutation being within the light chain. In those sequences the VL is correct, even though these sequences are from the heavy chain. Thus, the VL_X ##Y refers to the position of the tyrosine mutation within the antibody, regardless of whether the sequence is a heavy or light chain. As shown in Table 7, several individual tyrosine substitutions demonstrated modestly improved off-rate (1.5 to 2.6 folds over parental 20E6 Fab control).

TABLE 6
20E6 variants containing single tyrosine substitutions in selected CDR positions

| Seq | | |
|---|---|---|
| ID | Identifier | Amino acid sequence |
| 95 | VL R24Y (38BIT)_HCDR1 | SYWMH |
| 96 | VL R24Y (38BIT)_HCDR2 | RIDPQSGGIKYAQKFQG |
| 97 | VL R24Y (38BIT)_HCDR3 | WDYGGYFDV |
| 98 | VL R24Y (38BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 99 | VL R24Y (38BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 100 | VL R24Y (38BIT)_LCDR1 | YASQDITNYLN |
| 101 | VL R24Y (38BIT)_LCDR2 | YTSRLHS |
| 102 | VL R24Y (38BIT)_LCDR3 | QQGDTLPWT |
| 103 | VL R24Y (38BIT)_VL | DIQMTQSPSSLSASVGDRVTITCYASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 104 | VL R24Y (38BIT)_LC | DIQMTQSPSSLSASVGDRVTITCYASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 105 | VL A25Y (39BIT)_HCDR1 | SYWMH |
| 106 | VL A25Y (39BIT)_HCDR2 | RIDPQSGGIKYAQKFQG |
| 107 | VL A25Y (39BIT)_HCDR3 | WDYGGYFDV |
| 108 | VL A25Y (39BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 109 | VL A25Y (39BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 110 | VL A25Y (39BIT)_LCDR1 | RYSQDITNYLN |
| 111 | VL A25Y (39BIT)_LCDR2 | YTSRLHS |
| 112 | VL A25Y (39BIT)_LCDR3 | QQGDTLPWT |
| 113 | VL A25Y (39BIT)_VL | DIQMTQSPSSLSASVGDRVTITCRYSQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 114 | VL A25Y (39BIT)_LC | DIQMTQSPSSLSASVGDRVTITCRYSQDITNYLNWYQQKP |
| | | GKAVKLLIYYTSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 115 | VL S26Y (40BIT)_HCDR1 | SYWMH |
| 116 | VL S26Y (40BIT)_HCDR2 | RIDPQSGGIKYAQKFQG |
| 117 | VL S26Y (40BIT)_HCDR3 | WDYGGYFDV |
| 118 | VL S26Y (40BIT )_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 119 | VL S26Y (40BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 120 | VL S26Y (40BIT)_LCDR1 | RAYQDITNYLN |
| 121 | VL S26Y (40BIT)_LCDR2 | YTSRLHS |
| 122 | VL S26Y (40BIT)_LCDR3 | QQGDTLPWT |
| 123 | VL S26Y (40BIT)_VL | DIQMTQSPSSLSASVGDRVTITCRAYQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 124 | VL S26Y (40BIT)_LC | DIQMTQSPSSLSASVGDRVTITCRAYQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 125 | VL Q27Y (41BIT)_HCDR1 | SYWMH |
| 126 | VL Q27Y (41BIT)_HCDR2 | RIDPQSGGIKYAQKFQG |
| 127 | VL Q27Y (41BIT)_HCDR3 | WDYGGYFDV |
| 128 | VL Q27Y (41BIT)_VH | EVOLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 129 | VL Q27Y (41BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 130 | VL Q27Y (41BIT)_LCDR1 | RASYDITNYLN |
| 131 | VL Q27Y (41BIT)_LCDR2 | YTSRLHS |
| 132 | VL Q27Y (41BIT)_LCDR3 | QQGDTLPWT |
| 133 | VL Q27Y (41BIT)_VL | DIQMTQSPSSLSASVGDRVTITCRASYDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 134 | VL Q27Y (41BIT)_LC | DIQMTQSPSSLSASVGDRVTITCRASYDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 135 | VL D28Y (42BIT)_HCDR1 | SYWMH |
| 136 | VL D28Y (42BIT)_HCDR2 | RIDPQSGGIKYAQKFQG |
| 137 | VL D28Y (42BIT)_HCDR3 | WDYGGYFDV |
| 138 | VL D28Y (42BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 139 | VL D28Y (42BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 140 | VL D28Y (42BIT)_LCDR1 | RASQYITNYLN |
| 141 | VL D28Y (42BIT)_LCDR2 | YTSRLHS |
| 142 | VL D28Y (42BIT)_LCDR3 | QQGDTLPWT |
| 143 | VL D28Y (42BIT)_VL | DIQMTQSPSSLSASVGDRVTITCRASQYITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 144 | VL D28Y (42BIT)_LC | DIQMTQSPSSLSASVGDRVTITCRASQYITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 145 | VL I29Y (43BIT)_HCDR1 | SYWMH |
| 146 | VL I29Y (43BIT)_HCDR2 | RIDPQSGGIKYAQKFQG |
| 147 | VL I29Y (43BIT)_HCDR3 | WDYGGYFDV |
| 148 | VL I29Y (43BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 149 | VL I29Y (43BIT)_Fab_HC | EVOLVQSGAEVKKPGASVKVSCKASGYTFTSYWMHWVRQA |
| | | PGOGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 150 | VL I29Y (43BIT)_LCDR1 | RASQDYTNYLN |
| 151 | VL I29Y (43BIT)_LCDR2 | YTSRLHS |
| 152 | VL I29Y (43BIT)_LCDR3 | QQGDTLPWT |
| 153 | VL I29Y (43BIT)_VL | DIQMTQSPSSLSASVGDRVTITCRASQDYTNYLNWYQQKP |
| | | GKAVKLLIYYTSRLHSGVPSRESGSGSGTDYTLTISSLQP |
| | | EDFATYFCQQGDTLPWTFGQGTKLEIK |
| 154 | VL I29Y (43BIT)_LC | DIQMTQSPSSLSASVGDRVTITCRASQDYTNYLNWYQQKP |
| | | GKAVKLLIYYTSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 155 | VL T30Y (44BIT)_HCDR1 | SYWMH |
| 156 | VL T30Y (44BIT)_HCDR2 | RIDPQSGGIKYAQKFQG |
| 157 | VL T30Y (44BIT)_HCDR3 | WDYGGYFDV |
| 158 | VL T30Y (44BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 159 | VL T30Y (44BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 160 | VL T30Y (44BIT)_LCDR1 | RASQDIYNYLN |
| 161 | VL T30Y (44BIT)_LCDR2 | YTSRLHS |
| 162 | VL T30Y (44BIT)_LCDR3 | QQGDTLPWT |
| 163 | VL T30Y (44BIT)_VL | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIK |
| 164 | VL T30Y (44BIT)_LC | DIQMTQSPSSLSASVGDRVTITCRASQDIYNYLNWYQQKP |
| | | GKAVKLLIYYTSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 165 | VL N31Y (45BIT)_HCDR1 | SYWMH |
| 166 | VL N31Y (45BIT)_HCDR2 | RIDPQSGGIKYAQKFQG |
| 167 | VL N31Y (45BIT)_HCDR3 | WDYGGYFDV |
| 168 | VL N31Y (45BIT)_VH | EVOLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 169 | VL N31Y (45BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 170 | VL N31Y (45BIT)_LCDR1 | RASQDITYYLN |
| 171 | VL N31Y | YTSRLHS |
| | (45BIT)_LCDR2 | |
| 172 | L N31Y (45BIT)_LCDR3 | QQGDTLPWT |
| 173 | VL N31Y (45BIT)_VL | DIQMTQSPSSLSASVGDRVTITCRASQDITYYLNWYQQKP |
| | | GKAVKLLIYYTSRLHSGVPSRFSGSGSGTDYTLTISSLQP |
| | | EDFATYFCQQGDTLPWTFGQGTKLEIK |
| 174 | VL N31Y (45BIT)_LC | DIQMTQSPSSLSASVGDRVTITCRASQDITYYLNWYQQKP |
| | | GKAVKLLIYYTSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 175 | VL L33Y (46BIT)_HCDR1 | SYWMH |
| 176 | VL L33Y (46BIT)_HCDR2 | RIDPQSGGIKYAQKFQG |
| 177 | VL L33Y (46BIT)_HCDR3 | WDYGGYFDV |
| 178 | VL L33Y (46BIT)_VH | EVOLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 179 | VL L33Y (46BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 180 | VL L33Y (46BIT)_LCDR1 | RASQDITNYYN |
| 181 | VL L33Y (46BIT)_LCDR2 | YTSRLHS |
| 182 | VL L33Y (46BIT) | QQGDTLPWT |
| | LCDR3 | |
| 183 | VL L33Y (46BIT)_VL | DIQMTQSPSSLSASVGDRVTITCRASQDITNYYNWYQQKP |
| | | GKAVKLLIYYTSRLHSGVPSRESGSGSGTDYTLTISSLQP |
| | | EDFATYFCQQGDTLPWTFGQGTKLEIK |
| 184 | VL L33Y (46BIT)_LC | DIQMTQSPSSLSASVGDRVTITCRASQDITNYYNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 185 | VL N34Y (47BIT)_HCDR1 | SYWMH |
| 186 | VL N34Y (47BIT)_HCDR2 | RIDPQSGGIKYAQKFQG |
| 187 | VL N34Y (47BIT)_HCDR3 | WDYGGYFDV |
| 188 | VL N34Y (47BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 189 | VL N34Y (47BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 190 | VL N34Y (47BIT)_LCDR1 | RASQDITNYLY |
| 191 | VL N34Y (47BIT)_LCDR2 | YTSRLHS |
| 192 | VL N34Y (47BIT)_LCDR3 | QQGDTLPWT |
| 193 | VL N34Y (47BIT)_VL | DIQMTQSPSSLSASVGDRVTITCRASQDITNYLYWYQQKP |
| | | GKAVKLLIYYTSRLHSGVPSRFSGSGSGTDYTLTISSLQP |
| | | EDFATYFCQQGDTLPWTFGQGTKLEIK |
| 194 | VL N34Y (47BIT)_LC | DIQMTQSPSSLSASVGDRVTITCRASQDITNYLYWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 195 | VL T51Y (48BIT)_HCDR1 | SYWMH |
| 196 | VL T51Y (48BIT)_HCDR2 | RIDPQSGGIKYAQKFQG |
| 197 | VL T51Y (48BIT)_HCDR3 | WDYGGYFDV |
| 198 | VL T51Y (48BIT)_VH | EVOLVQSGAEVKKPGASVKVSCKASGYTETSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 199 | VL T51Y (48BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLOSSGLYSLSSVVTVPSSSLGTQTYI |
| | | CNVNHKPSNTKVDKKVEPKSCDKTHT |
| 200 | VL T51Y (48BIT)_LCDR1 | RASQDITNYLN |
| 201 | VL T51Y (48BIT)_LCDR2 | YYSRLHS |
| 202 | VL T51Y (48BIT)_LCDR3 | QQGDTLPWT |
| 203 | VL T51Y (48BIT)_VL | DIQMTQSPSSLSASVGDRVTITCRASQDITNYLNWYQQKP |
| | | GKAVKLLIYYYSRLHSGVPSRFSGSGSGTDYTLTISSLQP |
| | | EDFATYFCQQGDTLPWTFGQGTKLEIK |
| 204 | VL T51Y (48BIT)_LC | DIQMTQSPSSLSASVGDRVTITCRASQDITNYLNWYQQKP |
| | | GKAVKLLIYYYSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 205 | VL S52Y (49BIT)_HCDR1 | SYWMH |
| 206 | VL S52Y (49BIT)_HCDR2 | RIDPQSGGIKYAQKFQG |
| 207 | VL S52Y (49BIT)_HCDR3 | WDYGGYFDV |
| 208 | VL S52Y (49BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 209 | VL S52Y (49BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 210 | VL S52Y (49BIT)_LCDR1 | RASQDITNYLN |
| 211 | VL S52Y (49BIT)_LCDR2 | YTYRLHS |
| 212 | VL S52Y (49BIT)_LCDR3 | QQGDTLPWT |
| 213 | VL S52Y (49BIT)_VL | DIQMTQSPSSLSASVGDRVTITCRASQDITNYLNWYQQKP |
| | | GKAVKLLIYYTYRLHSGVPSRESGSGSGTDYTLTISSLQP |
| | | EDFATYFCQQGDTLPWTFGQGTKLEIK |
| 214 | VL S52Y (49BIT)_LC | DIQMTQSPSSLSASVGDRVTITCRASQDITNYLNWYQQKP |
| | | GKAVKLLIYYTYRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 215 | VL R53Y (50BIT)_HCDR1 | SYWMH |
| 216 | VL R53Y (50BIT)_HCDR2 | RIDPQSGGIKYAQKFQG |
| 217 | VL R53Y (50BIT)_HCDR3 | WDYGGYFDV |
| 218 | VL R53Y (50BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 219 | VL R53Y (50BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 220 | VL R53Y (50BIT)_LCDR1 | RASQDITNYLN |
| 221 | VL R53Y (50BIT)_LCDR2 | YTSYLHS |
| 222 | VL R53Y (50BIT)_LCDR3 | QQGDTLPWT |
| 223 | VL R53Y (50BIT)_VL | DIQMTQSPSSLSASVGDRVTITCRASQDITNYLNWYQQKP |
| | | GKAVKLLIYYTSYLHSGVPSRFSGSGSGTDYTLTISSLQP |
| | | EDFATYFCQQGDTLPWTFGQGTKLEIK |
| 224 | VL R53Y (50BIT)_LC | DIQMTQSPSSLSASVGDRVTITCRASQDITNYLNWYQQKP |
| | | GKAVKLLIYYTSYLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 225 | VL L54Y (51BIT)_HCDR1 | SYWMH |
| 226 | VL L54Y (51BIT)_HCDR2 | RIDPQSGGIKYAQKFQG |
| 227 | VL L54Y (51BIT)_HCDR3 | WDYGGYFDV |
| 228 | VL L54Y (51BIT)_VH | EVOLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 229 | VL L54Y (51BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 230 | VL L54Y (51BIT)_LCDR1 | RASQDITNYLN |
| 231 | VL L54Y (51BIT)_LCDR2 | YTSRYHS |
| 232 | VL L54Y (51BIT)_LCDR3 | QQGDTLPWT |
| 233 | VL L54Y (51BIT)_VL | DIQMTQSPSSLSASVGDRVTITCRASQDITNYLNWYQQKP |
| | | GKAVKLLIYYTSRYHSGVPSRFSGSGSGTDYTLTISSLQP |
| | | EDFATYFCQQGDTLPWTFGQGTKLEIK |
| 234 | VL L54Y (51BIT)_LC | DIQMTQSPSSLSASVGDRVTITCRASQDITNYLNWYQQKP |
| | | GKAVKLLIYYTSRYHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVOWKVDNALQSGNSQ |
| | | ESVTEQDSKDSTYSLSSTLTLSKADYEKHKVYACEVTHQG |
| | | LSSPVTKSFNRGEC |
| 235 | VL H55Y (52BIT)_HCDR1 | SYWMH |
| 236 | VL H55Y (52BIT)_HCDR2 | RIDPQSGGIKYAQKFQG |
| 237 | VL H55Y (52BIT)_HCDR3 | WDYGGYFDV |
| 238 | VL H55Y (52BIT)_VH | EVOLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 239 | VL H55Y (52BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 240 | VL H55Y (52BIT)_LCDR1 | RASQDITNYLN |
| 241 | VL H55Y (52BIT)_LCDR2 | YTSRLYS |
| 242 | VL H55Y (52BIT)_LCDR3 | QQGDTLPWT |
| 243 | VL H55Y (52BIT)_VL | DIQMTQSPSSLSASVGDRVTITCRASQDITNYLNWYQQKP |
| | | GKAVKLLIYYTSRLYSGVPSRFSGSGSGTDYTLTISSLQP |
| | | EDFATYFCQQGDTLPWTFGQGTKLEIK |
| 244 | VL H55Y (52BIT)_LC | DIQMTQSPSSLSASVGDRVTITCRASQDITNYLNWYQQKP |
| | | GKAVKLLIYYTSRLYSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 245 | VL S56Y (53BIT)_HCDR1 | SYWMH |
| 246 | VL S56Y (53BIT)_HCDR2 | RIDPQSGGIKYAQKFQG |
| 247 | VL S56Y (53BIT)_HCDR3 | WDYGGYFDV |
| 248 | VL S56Y (53BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 249 | VL S56Y (53BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 250 | VL S56Y (53BIT)_LCDR1 | RASQDITNYLN |
| 251 | VL S56Y (53BIT)_LCDR2 | YTSRLHY |
| 252 | VL S56Y (53BIT)_LCDR3 | QQGDTLPWT |
| 253 | VL S56Y (53BIT)_VL | DIQMTQSPSSLSASVGDRVTITCRASQDITNYLNWYQQKP |
| | | GKAVKLLIYYTSRLHYGVPSRFSGSGSGTDYTLTISSLQP |
| | | EDFATYFCQQGDTLPWTFGQGTKLEIK |
| 254 | VL S56Y (53BIT)_LC | DIQMTQSPSSLSASVGDRVTITCRASQDITNYLNWYQQKP |
| | | GKAVKLLIYYTSRLHYGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 255 | VL Q89Y (54BIT)_HCDR1 | SYWMH |
| 256 | VL Q89Y (54BIT)_HCDR2 | RIDPQSGGIKYAQKFQG |
| 257 | VL Q89Y (54BIT)_HCDR3 | WDYGGYFDV |
| 258 | VL Q89Y (54BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 259 | VL Q89Y (54BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 260 | VL Q89Y (54BIT)_LCDR1 | RASQDITNYLN |
| 261 | VL Q89Y (54BIT)_LCDR2 | YTSRLHS |
| 262 | VL Q89Y (54BIT)_LCDR3 | YQGDTLPWT |
| 263 | VL Q89Y (54BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCYQGDTLPWTFGQGTKLEIK |
| 264 | VL Q89Y (54BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCYQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 265 | VL Q90Y (55BIT)_HCDR1 | SYWMH |
| 266 | VL Q90Y (55BIT)_HCDR2 | RIDPQSGGIKYAQKFQG |
| 267 | VL Q90Y (55BIT)_HCDR3 | WDYGGYFDV |
| 268 | VL Q90Y (55BIT)_VH | EVOLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 269 | VL Q90Y (55BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 270 | VL Q90Y (55BIT)_LCDR1 | RASQDITNYLN |
| 271 | VL Q90Y (55BIT)_LCDR2 | YTSRLHS |
| 272 | VL Q90Y (55BIT)_LCDR3 | QYGDTLPWT |
| 273 | VL Q90Y (55BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQYGDTLPWTFGQGTKLEIK |
| 274 | VL Q90Y (55BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQYGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 275 | VL G91Y (56BIT)_HCDR1 | SYWMH |
| 276 | VL G91Y (56BIT)_HCDR2 | RIDPQSGGIKYAQKFQG |
| 277 | VL G91Y (56BIT)_HCDR3 | WDYGGYFDV |
| 278 | VL G91Y (56BIT)_VH | EVOLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 279 | VL G91Y (56BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 280 | VL G91Y (56BIT)_LCDR1 | RASQDITNYLN |
| 281 | VL G91Y (56BIT)_LCDR2 | YTSRLHS |
| 282 | VL G91Y (56BIT)_LCDR3 | QQYDTLPWT |
| 283 | VL G91Y (56BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQYDTLPWTFGQGTKLEIK |
| 284 | VL G91Y (56BIT)_LC | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQYDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 285 | VL D92Y (57BIT)_HCDR1 | SYWMH |
| 286 | VL D92Y (57BIT)_HCDR2 | RIDPQSGGIKYAQKFQG |
| 287 | VL D92Y (57BIT)_HCDR3 | WDYGGYFDV |
| 288 | VL D92Y (57BIT)_VH | EVOLVQSGAEVKKPGASVKVSCKASGYTETSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 289 | VL D92Y (57BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 290 | VL D92Y (57BIT)_LCDR1 | RASQDITNYLN |
| 291 | VL D92Y (57BIT)_LCDR2 | YTSRLHS |
| 292 | VL D92Y (57BIT)_LCDR3 | QQGYTLPWT |
| 293 | VL D92Y (57BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGYTLPWTFGQGTKLEIK |
| 294 | VL D92Y (57BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGYTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 295 | VL T93Y (58BIT)_HCDR1 | SYWMH |
| 296 | VL T93Y (58BIT)_HCDR2 | RIDPQSGGIKYAQKFQG |
| 297 | VL T93Y | WDYGGYFDV |
| | (58BIT)_HCDR3 | |
| 298 | VL T93Y (58BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 299 | VL T93Y (58BIT )_Fab_HC | EVOLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 300 | VL T93Y (58BIT)_LCDR1 | RASQDITNYLN |
| 301 | VL T93Y (58BIT)_LCDR2 | YTSRLHS |
| 302 | VL T93Y (58BIT)_LCDR3 | QQGDYLPWT |
| 303 | VL T93Y (58BIT)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDYLPWTFGQGTKLEIK |
| 304 | VL T93Y (58BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDYLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 305 | VL L94Y (59BIT)_HCDR1 | SYWMH |
| 306 | VL L94Y (59BIT)_HCDR2 | RIDPQSGGIKYAQKFQG |
| 307 | VL L94Y (59BIT)_HCDR3 | WDYGGYFDV |
| 308 | VL L94Y (59BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 309 | VL L94Y (59BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 310 | VL L94Y (59BIT)_LCDR1 | RASQDITNYLN |
| 311 | VL L94Y (59BIT)_LCDR2 | YTSRLHS |
| 312 | VL L94Y (59BIT)_LCDR3 | QQGDTYPWT |
| 313 | VL L94Y (59BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTYPWTFGQGTKLEIK |
| 314 | VL L94Y (59BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTYPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 315 | VL P95Y (60BIT)_HCDR1 | SYWMH |
| 316 | VL P95Y (60BIT)_HCDR2 | RIDPQSGGIKYAQKFQG |
| 317 | VL P95Y (60BIT)_HCDR3 | WDYGGYFDV |
| 318 | VL P95Y (60BIT)_VH | EVQLVQSGAEVKKPGASVKVSCKASGYTETSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 319 | VL P95Y (60BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 320 | VL P95Y (60BIT)_LCDR1 | RASQDITNYLN |
| 321 | VL P95Y (60BIT)_LCDR2 | YTSRLHS |
| 322 | VL P95Y (60BIT)_LCDR3 | QQGDTLYWT |
| 323 | VL P95Y (60BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLYWTFGQGTKLEIK |
| 324 | VL P95Y (60BIT)_LC | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLYWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 325 | VH T28Y (67BIT)_HCDR1 | SYWMH |
| 326 | VH T28Y (67BIT)_HCDR2 | RIDPQSGGIKYAQKFQG |
| 327 | VH T28Y (67BIT)_HCDR3 | WDYGGYFDV |
| 328 | VH T28Y (67BIT)_VH | EVOLVQSGAEVKKPGASVKVSCKASGYYFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 329 | VH T28Y (67BIT)_Fab_HC | EVQLVQSGAEVKKPGASVKVSCKASGYYFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 330 | VH T28Y (67BIT)_LCDR1 | RASQDITNYLN |
| 331 | VH T28Y (67BIT)_LCDR2 | YTSRLHS |
| 332 | VH T28Y (67BIT)_LCDR3 | QQGDTLPWT |
| 333 | VH T28Y (67BIT)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIK |
| 334 | VH T28Y (67BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 335 | VH F29Y (68BIT)_HCDR1 | SYWMH |
| 336 | VH F29Y (68BIT)_HCDR2 | RIDPQSGGIKYAQKFQG |
| 337 | VH F29Y (68BIT)_HCDR3 | WDYGGYFDV |
| 338 | VH F29Y (68BIT)_VH | EVOLVQSGAEVKKPGASVKVSCKASGYTYTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 339 | VH F29Y (68BIT)_Fab_HC | EVQLVQSGAEVKKPGASVKVSCKASGYTYTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVHQSSGLYSLSSVVTVPSSSLGTQTYI |
| | | CNVNHKPSNTKVDKKVEPKSCDKTHT |
| 340 | VH F29Y (68BIT)_LCDR1 | RASQDITNYLN |
| 341 | VH F29Y (68BIT)_LCDR2 | YTSRLHS |
| 342 | VH F29Y (68BIT)_LCDR3 | QQGDTLPWT |
| 343 | VH F29Y (68BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 344 | VH F29Y (68BIT)_LC | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 345 | VH T30Y (69BIT)_HCDR1 | SYWMH |
| 346 | VH T30Y (69BIT)_HCDR2 | RIDPQSGGIKYAQKFQG |
| 347 | VH T30Y (69BIT)_HCDR3 | WDYGGYFDV |
| 348 | VH T30Y (69BIT)_VH | EVOLVQSGAEVKKPGASVKVSCKASGYTFYSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 349 | VH T30Y (69BIT)_Fab_HC | EVQLVQSGAEVKKPGASVKVSCKASGYTFYSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 350 | VH T30Y (69BIT)_LCDR1 | RASQDITNYLN |
| 351 | T30Y (69BIT LCDR2 | YTSRLHS |
| 352 | T30Y (69BIT) | QQGDTLPWT |
| | LCDR3 | |
| 353 | VH T30Y (69BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 354 | VH T30Y (69BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 355 | VH S31Y (70BIT)_HCDR1 | YYWMH |
| 356 | VH S31Y (70BIT)_HCDR2 | RIDPQSGGIKYAQKFQG |
| 357 | VH S31Y (70BIT)_HCDR3 | WDYGGYFDV |
| 358 | VH S31Y (70BIT)_VH | EVQLVQSGAEVKKPGASVKVSCKASGYTFTYYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 359 | VH S31Y (70BIT)_Fab_HC | EVQLVQSGAEVKKPGASVKVSCKASGYTFTYYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 360 | VH S31Y (70BIT)_LCDR1 | RASQDITNYLN |
| 361 | VH S31Y (70BIT)_LCDR2 | YTSRLHS |
| 362 | VH S31Y (70BIT)_LCDR3 | QQGDTLPWT |
| 363 | VH S31Y (70BIT)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIK |
| 364 | VH S31Y (70BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVOWKVDNALQSGNSQ |
| | | ESVTEQDSKDSTYSLSSTLTLSKADYEKHKVYACEVTHQG |
| | | LSSPVTKSFNRGEC |
| 365 | VH W33Y (71BIT)_HCDR1 | SYYMH |
| 366 | VH W33Y (71BIT)_HCDR2 | RIDPQSGGIKYAQKFQG |
| 367 | VH W33Y (71BIT)_HCDR3 | WDYGGYFDV |
| 368 | VH W33Y (71BIT)_VH | EVQLVQSGAEVKKPGASVKVSCKASGYTFTSYYMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 369 | VH W33Y (71BIT)_Fab_HC | EVQLVQSGAEVKKPGASVKVSCKASGYTFTSYYMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 370 | VH W33Y (71BIT)_LCDR1 | RASQDITNYLN |
| 371 | VH W33Y (71BIT)_LCDR2 | YTSRLHS |
| 372 | VH W33Y (71BIT)_LCDR3 | QQGDTLPWT |
| 373 | VH W33Y (71BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 374 | VH W33Y (71BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 375 | VH M34Y (72BIT)_HCDR1 | SYWYH |
| 376 | VH M34Y (72BIT)_HCDR2 | RIDPQSGGIKYAQKFQG |
| 377 | VH M34Y (72BIT)_HCDR3 | WDYGGYFDV |
| 378 | VH M34Y (72BIT)_VH | EVQLVQSGAEVKKPGASVKVSCKASGYTFTSYWYHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 379 | VH M34Y (72BIT)_Fab_HC | EVQLVQSGAEVKKPGASVKVSCKASGYTFTSYWYHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 380 | VH M34Y (72BIT)_LCDR1 | RASQDITNYLN |
| 381 | VH M34Y (72BIT)_LCDR2 | YTSRLHS |
| 382 | VH M34Y (72BIT)_LCDR3 | QQGDTLPWT |
| 383 | VH M34Y (72BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 384 | VH M34Y (72BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 385 | VH H35Y (73BIT)_HCDR1 | SYWMY |
| 386 | VH H35Y (73BIT)_HCDR2 | RIDPQSGGIKYAQKFQG |
| 387 | VH H35Y (73BIT)_HCDR3 | WDYGGYFDV |
| 388 | VH H35Y (73BIT)_VH | EVQLVQSGAEVKKPGASVKVSCKASGYTFTSYWMYWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 389 | VH H35Y (73BIT)_Fab_HC | EVQLVQSGAEVKKPGASVKVSCKASGYTFTSYWMYWVRQA |
| | | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 390 | VH H35Y (73BIT)_LCDR1 | RASQDITNYLN |
| 391 | VH H35Y (73BIT)_LCDR2 | YTSRLHS |
| 392 | VH H35Y (73BIT)_LCDR3 | QQGDTLPWT |
| 393 | VH H35Y (73BIT)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIK |
| 394 | VH H35Y (73BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 395 | VH R50Y (75BIT)_HCDR1 | SYWYH |
| 396 | VH R50Y (75BIT)_HCDR2 | YIDPQSGGIKYAQKFQG |
| 397 | VH R50Y (75BIT)_HCDR3 | WDYGGYFDV |
| 398 | VH R50Y (75BIT)_VH | EVQLVQSGAEVKKPGASVKVSCKASGYTFTSYWMHWVRQA |
| | | PGQGLEWMGYIDPQSGGIKYAQKFQGRATLTVDTSTSTAY |
| | | MELSRLRSDDTAVYYCARWDYGGYFDVWGQGTLVTVSS |
| 399 | VH R50Y (75BIT)_Fab_HC | EVQLVQSGAEVKKPGASVKVSCKASGYTFTSYWMHWVRQA |
| | | PGQGLEWMGYIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 400 | VH R50Y (75BIT)_LCDR1 | RASQDITNYLN |
| 401 | VH R50Y (75BIT)_LCDR2 | YTSRLHS |
| 402 | VH R50Y (75BIT)_LCDR3 | QQGDTLPWT |
| 403 | VH R50Y (75BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 404 | VH R50Y (75BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 405 | VH I51Y (76BIT)_HCDR1 | SYWYH |
| 406 | VH I51Y (76BIT)_HCDR2 | RYDPQSGGIKYAQKFQG |
| 407 | VH 151Y (76BIT)_HCDR3 | WDYGGYFDV |
| 408 | VH 151Y (76BIT)_VH | EVOLVQSGAEVKKPGASVKVSCKASGYTFTSYWMHWVRQA |
| | | PGQGLEWMGRYDPQSGGIKYAQKFQGRATLTVDTSTSTAY |
| | | MELSRLRSDDTAVYYCARWDYGGYFDVWGQGTLVTVSS |
| 409 | VH I51Y (76BIT)_Fab_HC | EVQLVQSGAEVKKPGASVKVSCKASGYTFTSYWMHWVRQA |
| | | PGQGLEWMGRYDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 410 | VH I51Y (76BIT)_LCDR1 | RASQDITNYLN |
| 411 | VH 151Y (76BIT)_LCDR2 | YTSRLHS |
| 412 | VH I51Y (76BIT)_LCDR3 | QQGDTLPWT |
| 413 | VH I51Y (76BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 414 | VH 151Y (76BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 415 | VH D52Y (77BIT)_HCDR1 | SYWYH |
| 416 | VH D52Y (77BIT) | RIYPQSGGIKYAQKFQG |
| | HCDR 2 | |
| 417 | VH D52Y (77BIT)_HCDR3 | WDYGGYFDV |
| 418 | VH D52Y (77BIT)_VH | EVQLVQSGAEVKKPGASVKVSCKASGYTFTSYWMHWVRQA |
| | | PGQGLEWMGRIYPQSGGIKYAQKFQGRATLTVDTSTSTAY |
| | | MELSRLRSDDTAVYYCARWDYGGYFDVWGQGTLVTVSS |
| 419 | VH D52Y (77BIT)_Fab_HC | EVQLVQSGAEVKKPGASVKVSCKASGYTFTSYWMHWVRQA |
| | | PGQGLEWMGRIYPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 420 | VH D52Y (77BIT)_LCDR1 | RASQDITNYLN |
| 421 | VH D52Y (77BIT)_LCDR2 | YTSRLHS |
| 422 | VH D52Y (77BIT)_LCDR3 | QQGDTLPWT |
| 423 | VH D52Y (77BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 424 | VH D52Y (77BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 425 | VH P52aY (78BIT)_HCDR1 | SYWYH |
| 426 | VH P52aY (78BIT)_HCDR2 | RIDYQSGGIKYAQKFQG |
| 427 | VH P52aY (78BIT)_HCDR3 | WDYGGYFDV |
| 428 | VH P52aY (78BIT)_VH | EVQLVQSGAEVKKPGASVKVSCKASGYTFTSYWMHWVRQA |
| | | PGQGLEWMGRIDYQSGGIKYAQKFQGRATLTVDTSTSTAY |
| | | MELSRLRSDDTAVYYCARWDYGGYFDVWGQGTLVTVSS |
| 429 | VH P52aY | EVQLVQSGAEVKKPGASVKVSCKASGYTFTSYWMHWVRQA |
| | (78BIT)_Fab_HC | PGQGLEWMGRIDYQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 430 | VH P52aY (78BIT)_LCDR1 | RASQDITNYLN |
| 431 | VH P52aY (78BIT)_LCDR2 | YTSRLHS |
| 432 | VH P52aY (78BIT)_LCDR3 | QQGDTLPWT |
| 433 | VH P52aY (78BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 434 | VH P52aY (78BIT)_LC | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 435 | VH Q53Y (79BIT)_HCDR1 | SYWYH |
| 436 | VH Q53Y (79BIT)_HCDR2 | RIDPYSGGIKYAQKFQG |
| 437 | VH Q53Y (79BIT)_HCDR3 | WDYGGYFDV |
| 438 | VH Q53Y (79BIT)_VH | EVQLVQSGAEVKKPGASVKVSCKASGYTFTSYWMHWVRQA |
| | | PGQGLEWMGRIDPYSGGIKYAQKFQGRATLTVDTSTSTAY |
| | | MELSRLRSDDTAVYYCARWDYGGYFDVWGQGTLVTVSS |
| 439 | VH Q53Y (79BIT)_Fab_HC | EVQLVQSGAEVKKPGASVKVSCKASGYTFTSYWMHWVRQA |
| | | PGQGLEWMGRIDPYSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 440 | VH Q53Y (79BIT)_LCDR1 | RASQDITNYLN |
| 441 | VH Q53Y (79BIT)_LCDR2 | YTSRLHS |
| 442 | VH Q53Y | QQGDTLPWT |
| | (79BIT)_LCDR3 | |
| 443 | VH Q53Y | DIQMTQSPSSLSASVGDRVTITCRASQDITNYLNWYQQKP |
| | (79BIT)_VL | GKAVKLLIYYTSRLHSGVPSRFSGSGSGTDYTLTISSLQP |
| | | EDFATYFCQQGDTLPWTFGQGTKLEIK |
| 444 | VH Q53Y (79BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 445 | 20E6 | SYWMH |
| | (80BGJ/85BIT)_HCDR1 | |
| 446 | 20E6 | RIDPQSGGIKYAQKFQG |
| | (80BGJ/85BIT)_HCDR2 | |
| 447 | 20E6 | WDYGGYFDV |
| | (80BGJ/85BIT)_HCDR3 | |
| 448 | 20E6 (80BGJ/85BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 449 | 20E6 | EVQLVQSGAEVKKPGASVKVSCKASGYTFTSYWMHWVRQA |
| | (80BGJ/85BIT)_Fab_HC | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 450 | 20E6 | RASQDITNYLN |
| | (80BGJ/85BIT)_LCDR1 | |
| 451 | 20E6 | YTSRLHS |
| | (80BGJ/85BIT)_LCDR2 | |
| 452 | 20E6 | QQGDTLPWT |
| | (80BGJ/85BIT)_LCDR3 | |
| 453 | 20E6 (80BGJ/85BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 454 | 20E6 (80BGJ/85BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 455 | VH S54Y (80BIT)_HCDR1 | SYWYH |
| 456 | VH S54Y (80BIT)_HCDR2 | RIDPQYGGIKYAQKFQG |
| 457 | VH S54Y (80BIT)_HCDR3 | WDYGGYFDV |
| 458 | VH S54Y (80BIT)_VH | EVOLVQSGAEVKKPGASVKVSCKASGYTFTSYWMHWVRQA |
| | | PGQGLEWMGRIDPQYGGIKYAQKFQGRATLTVDTSTSTAY |
| | | MELSRLRSDDTAVYYCARWDYGGYFDVWGQGTLVTVSS |
| 459 | VH S54Y (80BIT)_Fab_HC | EVQLVQSGAEVKKPGASVKVSCKASGYTFTSYWMHWVRQA |
| | | PGQGLEWMGRIDPQYGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 460 | VH S54Y (80BIT)_LCDR1 | RASQDITNYLN |
| 461 | VH S54Y (80BIT)_LCDR2 | YTSRLHS |
| 462 | VH S54Y (80BIT)_LCDR3 | QQGDTLPWT |
| 463 | VH S54Y (80BIT)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIK |
| 464 | VH S54Y (80BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 465 | VH G55Y (81BIT)_HCDR1 | SYWYH |
| 466 | VH G55Y (81BIT)_HCDR2 | RIDPQSYGIKYAQKFQG |
| 467 | VH G55Y (81BIT)_HCDR3 | WDYGGYFDV |
| 468 | VH G55Y (81BIT)_VH | EVQLVQSGAEVKKPGASVKVSCKASGYTFTSYWMHWVRQA |
| | | PGQGLEWMGRIDPQSYGIKYAQKFQGRATLTVDTSTSTAY |
| | | MELSRLRSDDTAVYYCARWDYGGYFDVWGQGTLVTVSS |
| 469 | VH G55Y (81BIT)_Fab_HC | EVQLVQSGAEVKKPGASVKVSCKASGYTFTSYWMHWVRQA |
| | | PGQGLEWMGRIDPQSYGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 470 | VH G55Y (81BIT)_LCDR1 | RASQDITNYLN |
| 471 | VH G55Y (81BIT)_LCDR 2 | YTSRLHS |
| 472 | VH G55Y (81BIT)_LCDR3 | QQGDTLPWT |
| 473 | VH G55Y (81BIT)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIK |
| 474 | VH G55Y (81BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 475 | VH G56Y (82BIT)_HCDR1 | SYWYH |
| 476 | VH G56Y (82BIT)_HCDR2 | RIDPQSGYIKYAQKFQG |
| 477 | VH G56Y (82BIT)_HCDR3 | WDYGGYFDV |
| 478 | VH G56Y (82BIT)_VH | EVQLVQSGAEVKKPGASVKVSCKASGYTFTSYWMHWVRQA |
| | | PGQGLEWMGRIDPQSGYIKYAQKFQGRATLTVDTSTSTAY |
| | | MELSRLRSDDTAVYYCARWDYGGYFDVWGQGTLVTVSS |
| 479 | VH G56Y (82BIT)_Fab_HC | EVQLVQSGAEVKKPGASVKVSCKASGYTFTSYWMHWVRQA |
| | | PGQGLEWMGRIDPQSGYIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 480 | VH G56Y (82BIT)_LCDR1 | RASQDITNYLN |
| 481 | VH G56Y (82BIT)_LCDR2 | YTSRLHS |
| 482 | VH G56Y (82BIT)_LCDR3 | QQGDTLPWT |
| 483 | VH G56Y (82BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 484 | VH G56Y (82BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 485 | VH I57Y (83BIT)_HCDR1 | SYWYH |
| 486 | VH I57Y (83BIT)_HCDR2 | RIDPQSGGYKYAQKFQG |
| 487 | VH I57Y (83BIT)_HCDR3 | WDYGGYFDV |
| 488 | VH I57Y (83BIT)_VH | EVQLVQSGAEVKKPGASVKVSCKASGYTFTSYWMHWVRQA |
| | | PGQGLEWMGRIDPQSGGYKYAQKFQGRATLTVDTSTSTAY |
| | | MELSRLRSDDTAVYYCARWDYGGYFDVWGQGTLVTVSS |
| 489 | VH 157Y (83BIT)_Fab_HC | EVOLVQSGAEVKKPGASVKVSCKASGYTFTSYWMHWVRQA |
| | | PGOGLEWMGRIDPQSGGYKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 490 | VH I57Y (83BIT)_LCDR1 | RASQDITNYLN |
| 491 | VH 157Y (83BIT)_LCDR2 | YTSRLHS |
| 492 | VH I57Y (83BIT)_LCDR3 | QQGDTLPWT |
| 493 | VH 157Y (83BIT)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIK |
| 494 | VH I57Y (83BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 495 | VH K58Y (84BIT )_HCDR1 | SYWYH |
| 496 | VH K58Y (84BIT)_HCDR2 | RIDPQSGGIYYAQKFQG |
| 497 | VH K58Y (84BIT)_HCDR3 | WDYGGYFDV |
| 498 | VH K58Y (84BIT)_VH | EVOLVQSGAEVKKPGASVKVSCKASGYTFTSYWMHWVRQA |
| | | PGQGLEWMGRIDPQSGGIYYAQKFQGRATLTVDTSTSTAY |
| | | MELSRLRSDDTAVYYCARWDYGGYFDVWGQGTLVTVSS |
| 499 | VH K58Y (84BIT)_Fab_HC | EVQLVQSGAEVKKPGASVKVSCKASGYTFTSYWMHWVRQA |
| | | PGQGLEWMGRIDPQSGGIYYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 500 | VH K58Y (84BIT)_LCDR1 | RASQDITNYLN |
| 501 | VH K58Y (84BIT)_LCDR2 | YTSRLHS |
| 502 | VH K58Y (84BIT)_LCDR3 | QQGDTLPWT |
| 503 | VH K58Y (84BIT)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIK |
| 504 | VH K58Y (84BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 505 | VH W95Y (88BIT)_HCDR1 | SYWYH |
| 506 | VH W95Y (88BIT)_HCDR2 | RIDPQSGGIKYAQKFQG |
| 507 | VH W95Y (88BIT HCDR3 | YDYGGYFDV |
| 508 | VH W95Y (88BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARYDYGGYFDVWGQGTLVTVSS |
| 509 | VH W95Y (88BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARYDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 510 | VH W95Y (88BIT)_LCDR1 | RASQDITNYLN |
| 511 | VH W95Y (88BIT)_LCDR2 | YTSRLHS |
| 512 | VH W95Y (88BIT)_LCDR3 | QQGDTLPWT |
| 513 | VH W95Y (88BIT)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIK |
| 514 | VH W95Y (88BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 515 | VH D96Y (64BIT)_HCDR1 | SYWYH |
| 516 | VH D96Y (64BIT)_HCDR2 | RIDPQSGGIKYAQKFQG |
| 517 | VH D96Y (64BIT)_HCDR3 | WYYGGYFDV |
| 518 | VH D96Y (64BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWYYGGYFDVWGQGTLVTVSS |
| 519 | VH D96Y (64BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWYYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 520 | VH D96Y (64BIT)_LCDR1 | RASQDITNYLN |
| 521 | VH D96Y (64BIT)_LCDR2 | YTSRLHS |
| 522 | VH D96Y (64BIT)_LCDR3 | QQGDTLPWT |
| 523 | VH D96Y (64BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 524 | VH D96Y (64BIT)_LC | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 525 | VH G98Y (65BIT)_HCDR1 | SYWYH |
| 526 | VH G98Y (65BIT )_HCDR2 | RIDPQSGGIKYAQKFQG |
| 527 | VH G98Y (65BIT)_HCDR3 | WDYYGYFDV |
| 528 | VH G98Y (65BIT)_VH | EVOLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYYGYFDVWGQGTLVTVSS |
| 529 | VH G98Y (65BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYYGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 530 | VH G98Y (65BIT)_LCDR1 | RASQDITNYLN |
| 531 | VH G98Y (65BIT)_LCDR2 | YTSRLHS |
| 532 | VH G98Y (65BIT)_LCDR3 | QQGDTLPWT |
| 533 | VH G98Y (65BIT)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 534 | VH G98Y (65BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 535 | VH G99Y (66BIT)_HCDR1 | SYWYH |
| 536 | VH G99Y (66BIT)_HCDR2 | RIDPQSGGIKYAQKFQG |
| 537 | VH G99Y (66BIT)_HCDR3 | WDYGYYFDV |
| 538 | VH G99Y (66BIT)_VH | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGYYFDVWGQGTLVTVSS |
| 539 | VH G99Y (66BIT)_Fab_HC | EVQLVQSGAEVKKPGASVKVSCKASGYTFTSYWMHWVRQA |
| | | PGQGLEWMGRIDPQSGGIKYAQKFQGRATLTVDTSTSTAY |
| | | MELSRLRSDDTAVYYCARWDYGYYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 540 | VH G99Y (66BIT)_LCDR1 | RASQDITNYLN |
| 541 | VH G99Y (66BIT)_ | YTSRLHS |
| | LCDR2 | |
| 542 | VH G99Y (66BIT)_LCDR3 | QQGDTLPWT |
| 543 | VH G99Y (66BIT)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIK |
| 544 | VH G99Y (66BIT)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |

TABLE 7
ForteBio off-rate screen for tyrosine scanning mutants

| | Binding response | k_{off} | Fold |
|---|---|---|---|
| Identifier | (nm) | (×10⁻² s⁻¹) | change |
| 20E6 (80BGJ) | 0.253 | 4.68 | 1.0 |
| 20E6 (85BIT) | 0.580 | 3.81 | 1.1 |
| VL R24Y (38BIT) | 0.446 | 2.70 | 1.6 |
| VL A25Y (39BIT) | 0.043 | NB | NA |
| VL S26Y (40BIT) | 0.371 | 2.83 | 1.5 |
| VL Q27Y (41BIT) | 0.427 | 2.29 | 1.8 |
| VL D28Y (42BIT) | 0.207 | 3.18 | 1.3 |
| VL I29Y (43BIT) | 0.071 | 2.64 | 1.6 |
| VL T30Y (44BIT) | 0.472 | 1.62 | 2.6 |
| VL N31Y (45BIT) | 0.344 | 2.41 | 1.8 |
| VL L33Y (46BIT) | 0.095 | 4.13 | 1.0 |
| VL N34Y (47BIT) | 0.221 | 4.36 | 1.0 |
| VL T51Y (48BIT) | 0.204 | 5.08 | 0.8 |
| VL S52Y (49BIT) | 0.431 | 4.09 | 1.0 |
| VL R53Y (50BIT) | 0.365 | 2.70 | 1.6 |
| VL L54Y (51BIT) | 0.299 | 4.05 | 1.0 |
| VL H55Y (52BIT) | 0.265 | 3.85 | 1.1 |
| VL S56Y (53BIT) | 0.344 | 3.24 | 1.3 |
| VL Q89Y (54BIT) | 0.035 | NB | NA |
| VL Q90Y (55BIT) | 0.040 | NB | NA |
| VL G91Y (56BIT) | 0.044 | NB | NA |
| VL D92Y (57BIT) | 0.046 | NB | NA |
| VL T93Y (58BIT) | 0.586 | 2.73 | 1.5 |
| VL L94Y (59BIT) | 0.308 | 5.71 | 0.7 |
| VL P95Y (60BIT) | 0.015 | NB | NA |
| VH T28Y (67BIT) | 0.326 | 3.43 | 1.2 |
| VH F29Y (68BIT) | 0.597 | 3.36 | 1.3 |
| VH T30Y (69BIT) | 0.372 | 3.65 | 1.2 |
| VH S31Y (70BIT) | 0.696 | 3.61 | 1.2 |
| VH W33Y (71BIT) | 0.770 | 2.30 | 1.8 |
| VH M34Y (72BIT) | 0.623 | 4.14 | 1.0 |
| VH H35Y (73BIT) | 0.334 | 4.71 | 0.9 |
| VH RSOY (75BIT) | 0.324 | 4.45 | 0.9 |
| VH 151Y (76BIT) | 0.600 | 3.79 | 1.1 |
| VH D52Y (77BIT) | 0.042 | NB | NA |
| VH P52aY (78BIT) | 0.566 | 4.54 | 0.9 |
| VH Q53Y (79BIT) | 0.625 | 4.16 | 1.0 |
| VH S54Y (80BIT) | 0.588 | 4.06 | 1.0 |
| VH G55Y (81BIT) | 0.542 | 3.60 | 1.2 |
| VH G56Y (82BIT) | 0.569 | 4.36 | 1.0 |
| VH I57Y (83BIT) | 0.555 | 4.30 | 1.0 |
| VH K58Y (84BIT) | 0.339 | 3.27 | 1.3 |
| VH W95Y (88BIT) | 0.638 | 4.25 | 1.0 |
| VH D96Y (64BIT) | 0.102 | 7.46 | 0.6 |
| VH G98Y (65BIT) | 0.037 | NB | NA |
| VH G99Y (66BIT) | 0.055 | 4.76 | 0.9 |

NB: No binding observed
NA: Not applicable

### Example 6: Analysis of Double Tyrosine 20E6 Variants Binding Human LAP-TGFb1, 2 and 3 by Surface Plasmon Resonance

Further studies were performed in order to determine whether further affinity improvement may be achieved by combining two or more tyrosine substitutions. Accordingly, three additional 20E6 variants were generated that contained two CDR tyrosine substitutions (VH-W33Y/VL-T30Y, VH-W33Y/VL-R53Y, and VH-W33Y/NL-T93Y, Kabat numbering, Table 8) and these molecules were analyzed for their interactions with LAP-TGFβ1.

A Series S CM4 sensor chip (GE Healthcare, catalog BR100534) was immobilized with an anti-human Fc capture antibody following the kit protocol (GE Healthcare, catalog BR100839) on a Biacore T200 instrument with 1× HBS-EP+ (Teknova, catalog H8022). Kinetic binding interactions between human LAP-TGFβ isoform 1 and tyrosine scanning double mutants were performed in 1× HBS-EP4 with 0.1 mg/mL, bovine serum albumin (BSA) (Jackson Immunoresearch, catalog 001-000-162) at 25° C. Approximately 70-80 RU of human LAP-TGFβ-Fc isoform 1 was captured to the anti-human Fc surface followed by injection of 1:3 serially diluted Fab from 1000 nM to 1.37 nM and including a negative control 0 nM Fab. Binding specificity between human LAP-TGFβ isoforms 2 and 3 and the tyrosine scanning double mutants were performed in 1× HEPES-buffered saline with EDTA and surfactant (HBS)-EP)+with 0.1 mg/mL BSA at 25° C. Approximately 25-40 relative units (RU) of human LAP-TGFβ2 and 3 were captured to the anti-human Fc surface followed by injection of 1:4 serially diluted Fab from 1000 nM to 3.91 nM including a negative control 0 nM Fab. The binding data were double referenced by subtraction of signal from a reference (capture surface only) flow cell and the negative control 0 nM Fab injection. Binding rate constants were determined by fitting the data with a 1:1 binding model (GE Healthcare Biacore T200 Evaluation software 3.0). As shown in Table 9, antibody 20E6 binds to human LAP-TGFβ1 with nanomolar affinity, but no appreciable signal increase was observed for human LAP-TGFβ isoform 2 or 3. The tyrosine scanning double mutants VH33/VL30, VH33/VL53, and VH33/VL93 demonstrated 4 fold to 20 fold improved binding affinity to human LAP-TGFβ1 over antibody 20E6. Like antibody 20E6, the tyrosine scanning double mutants did not bind human LAP-TGFβ isoform 2 and 3.

TABLE 8
20E6 antibody variants

| Seq | | |
|---|---|---|
| ID | Identifier | Amino acid sequence |
| 545 | VH W33Y/VL T30Y | SYYMH |
| | (90BJM)_HCDR1 | |
| 546 | VH W33Y/VL T30Y | RIDPQSGGIKYAQKFQG |
| | (90BJM)_HCDR2 | |
| 547 | VH W33Y/VL T30Y | WDYGGYFDV |
| | (90BJM)_HCDR3 | |
| 548 | VH W33Y/VL T30Y | EVQLVQSGAEVKKPGASVKVSCKASGYTFTSYYMHWVRQA |
| | (90BJM)_VH | PGQGLEWMGRIDPQSGGIKYAQKFQGRATLTVDTSTSTAY |
| | | MELSRLRSDDTAVYYCARWDYGGYFDVWGQGTLVTVSS |
| 549 | VH W33Y/VL T30Y | EVQLVQSGAEVKKPGASVKVSCKASGYTFTSYYMHWVRQA |
| | (90BJM)_Fab_HC | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 550 | VH W33Y/VL T30Y | RASQDIYNYLN |
| | (90BJM)_LCDR1 | |
| 551 | VH W33Y/VL T30Y | YTSRLHS |
| | (90BJM)_LCDR2 | |
| 552 | VH W33Y/VL T30Y | QQGDTLPWT |
| | (90BJM)_LCDR3 | |
| 553 | VH W33Y/VL T30Y | DIQMTQSPSSLSASVGDRVTITCRASQDIYNYLNWYQQKP |
| | (90BJM)_VL | GKAVKLLIYYTSRLHSGVPSRFSGSGSGTDYTLTISSLQP |
| | | EDFATYFCQQGDTLPWTFGQGTKLEIK |
| 554 | VH W33Y/VL T30Y | DIQMTQSPSSLSASVGDRVTITCRASQDIYNYLNWYQQKP |
| | (90BJM)_LC | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 555 | VH W33Y/VL R53Y | SYYMH |
| | (91BJM)_HCDR1 | |
| 556 | VH W33Y/VL R53Y | RIDPQSGGIKYAQKFQG |
| | (91BJM)_HCDR2 | |
| 557 | VH W33Y/VL R53Y | WDYGGYFDV |
| | (91BJM)_HCDR3 | |
| 558 | VH W33Y/VL R53Y | EVQLVQSGAEVKKPGASVKVSCKASGYTFTSYYMHWVRQA |
| | (91BJM)_VH | PGQGLEWMGRIDPQSGGIKYAQKFQGRATLTVDTSTSTAY |
| | | MELSRLRSDDTAVYYCARWDYGGYFDVWGQGTLVTVSS |
| 559 | VH W33Y/VL R53Y | EVQLVQSGAEVKKPGASVKVSCKASGYTFTSYYMHWVRQA |
| | (91BJM)_Fab_HC | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 560 | VH W33Y/VL R53Y | RASQDITNYLN |
| | (91BJM)_LCDR1 | |
| 561 | VH W33Y/VL R53Y | YTSYLHS |
| | (91BJM)_LCDR2 | |
| 562 | VH W33Y/VL R53Y | QQGDTLPWT |
| | (91BJM)_LCDR3 | |
| 563 | VH W33Y/VL R53Y | DIQMTQSPSSLSASVGDRVTITCRASQDITNYLNWYQQKP |
| | (91BJM)_VL | GKAVKLLIYYTSYLHSGVPSRFSGSGSGTDYTLTISSLQP |
| | | EDFATYFCQQGDTLPWTFGQGTKLEIK |
| 564 | VH W33Y/VL R53Y | DIQMTQSPSSLSASVGDRVTITCRASQDITNYLNWYQQKP |
| | (91BJM)_LC | GKAVKLLIYYTSYLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 565 | VH W33Y/VL T93Y | SYYMH |
| | (92BJM)_HCDR1 | |
| 566 | VH W33Y/VL T93Y | RIDPQSGGIKYAQKFQG |
| | (92BJM)_HCDR2 | |
| 567 | VH W33Y/VL T93Y | WDYGGYFDV |
| | (92BJM)_HCDR3 | |
| 568 | VH W33Y/VL T93Y | EVQLVQSGAEVKKPGASVKVSCKASGYTFTSYYMHWVRQA |
| | (92BJM)_VH | PGQGLEWMGRIDPQSGGIKYAQKFQGRATLTVDTSTSTAY |
| | | MELSRLRSDDTAVYYCARWDYGGYFDVWGQGTLVTVSS |
| 569 | VH W33Y/VL T93Y | EVQLVQSGAEVKKPGASVKVSCKASGYTFTSYYMHWVRQA |
| | (92BJM)_Fab_HC | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 570 | VH W33Y/VL T93Y | RASQDITNYLN |
| | (92BJM)_LCDR1 | |
| 571 | VH W33Y/VL T93Y | YTSRLHS |
| | (92BJM)_LCDR2 | |
| 572 | VH W33Y/VL T93Y | QQGDYLPWT |
| | (92BJM)_LCDR3 | |
| 573 | VH W33Y/VL T93Y | DIQMTQSPSSLSASVGDRVTITCRASQDITNYLNWYQQKP |
| | (92BJM)_VL | GKAVKLLIYYTSRLHSGVPSRFSGSGSGTDYTLTISSLQP |
| | | EDFATYFCQQGDYLPWTFGQGTKLEIK |
| 574 | VH W33Y/VL T93Y | DIQMTQSPSSLSASVGDRVTITCRASQDITNYLNWYQQKP |
| | (92BJM)_LC | GKAVKLLIYYTSRLHSGVPSRFSGSGSGTDYTLTISSLQP |
| | | EDFATYFCQQGDYLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |

TABLE 9
Parameters for 20E6 and double tyrosine variants
binding to human LAP-TGFβ isoforms 1, 2, and 3

| | | | | | Fold |
|---|---|---|---|---|---|
| | | kₒₙ | k_{off} | k_{D} | improvement |
| ID | Species | (×10⁶ M⁻¹s⁻¹) | (×10⁻² s⁻¹) | (nM) | in K_{D} |
| 20E6 (80BGJ) | LAP-TGFβ1 | 1.45 ± 0.11 | 10.3 ± 2.39 | 81.2 ± 2.00 | 1.0 |
| | LAP-TGFβ2 | NB | NB | NB | NB |
| | LAP-TGFβ3 | NB | NB | NB | NB |
| VH W33Y/VL T30Y | LAP-TGFβ1 | 1.89 ± 0.04 | 0.75 ± 0.01 | 4.00 ± 0.06 | 20 |
| (90BJM) | LAP-TGFβ2 | NB | NB | NB | NB |
| | LAP-TGFβ3 | NB | NB | NB | NB |
| VH W33Y/VL R53Y | LAP-TGFβ1 | 0.91 ± 0.02 | 1.54 ± 0.01 | 11.8 ± 8.80 | 7.0 |
| (91BJM) | LAP-TGFβ2 | NB | NB | NB | NB |
| | LAP-TGFβ3 | NB | NB | NB | NB |
| VH W33Y/VL T93Y | LAP-TGFβ1 | 0.65 ± 0.01 | 1.34 ± n/a | 20.6 ± 0.15 | 4.0 |
| (92BJM) | LAP-TGFβ2 | NB | NB | NB | NB |
| | LAP-TGFβ3 | NB | NB | NB | NB |

All values reported as average ± standard deviation from triplicate measurements
NB: No binding observed

### Example 7: Construction of Rationally Designed Libraries for Selection of Affinity-Matured 20E6 Variants by Yeast Display Technology

To improve the affinity of humanized 20E6 by an in vitro library display and selection strategy, the investigators rationally designed 20E6 variant libraries with structural information derived from the 20E6/human LAP-TGFb1 complex generated by Cryo EM structural analysis. CDR positions were selected to introduce sequence diversity based on the following criteria: (1.) less than 5 angstroms from human LAP-TGFb1 in the immune complex; (2.) identification between the 20E6 antibody sequence and the human IGKV1-33*01 and IGHV1-2*05 germline sequences used for 20E6 humanization; and (3.) whether the CDR position is a somatic mutation hot spot in human IGKV1-33*01 and IGHV1-2*05.

The studies were performed to determine inter alia whether changes introduced to 20E6 CDR residues that are within 5 angstroms of LAP-TGFb1 would have a high probability of impacting the interaction between 20E6 and LAP-TGFb1. To minimize introducing potential immunogenicity in human, however, the studies excluded 20E6 CDR residues that were identical to IGKV1-33*01 sequences or IGHV1-2*05 sequences and were at positions rarely mutated in human antibodies from these two germlines. Conversely, sequence diversities were utilized at selected 20E6 CDR positions that are highly mutated in IGKV1-33*01- or to IGIHV1-2*05-derived human antibodies, which are available at IMGT (imgt.org), even if their distance from LAP-TGFb1 are greater than 5 angstroms. The selected 20E6 CDR residues for potential changes include light chain residues T30, N31, Y32, Y50, T51, R53, D92, T93, L94, and heavy chain S31, W33, M34, H35, Q53, S54, G56, 157, K58, W95, D96, Y97, G98, G99, Y100 (all Kabat numbering).

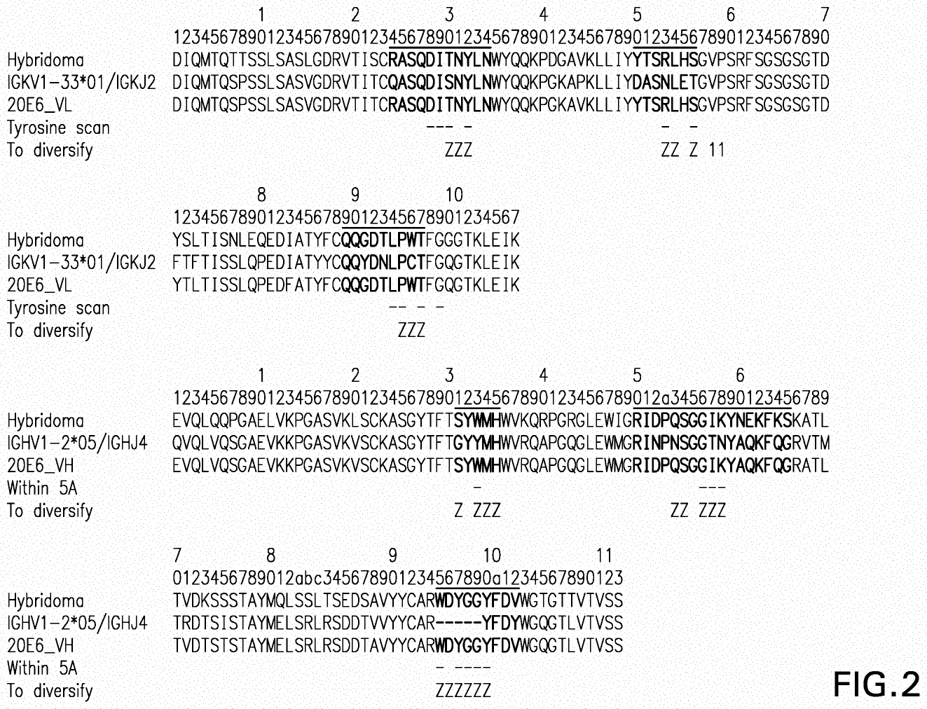

Different approaches were utilized to introduce diversity into selected CDR positions. In the first ‘doping’ approach, each of the three nucleotides of the selected CDR codon was synthesized at a ratio of 79% original and 7% each the other three nucleotides. In general, this 79:7:7:7 doping scheme would result in the CDR residue having an amino acid frequency distribution of approximately 60% parental residue and approximately 40% mutated to the other 19 amino acids and stop codon at uneven ratios, depending on number and position of sequence changes at the nucleotide level. Three ‘doped’ libraries for antibody 20E6 were constructed by routine recombinant DNA technologies containing CDR diversities in HCDR1 and 2, in HCDR3, and in all three LCDRs, respectively. In the doped light chain library, toggle mutations were included at positions 1-155E/S56T (Kabat numbering) in LCDR2 in an attempt to mutate the H55/S56 in 20E6 back to E55/T56 in IGKV1-33*01 in order to further increase identity to human sequence (FIG. 2).

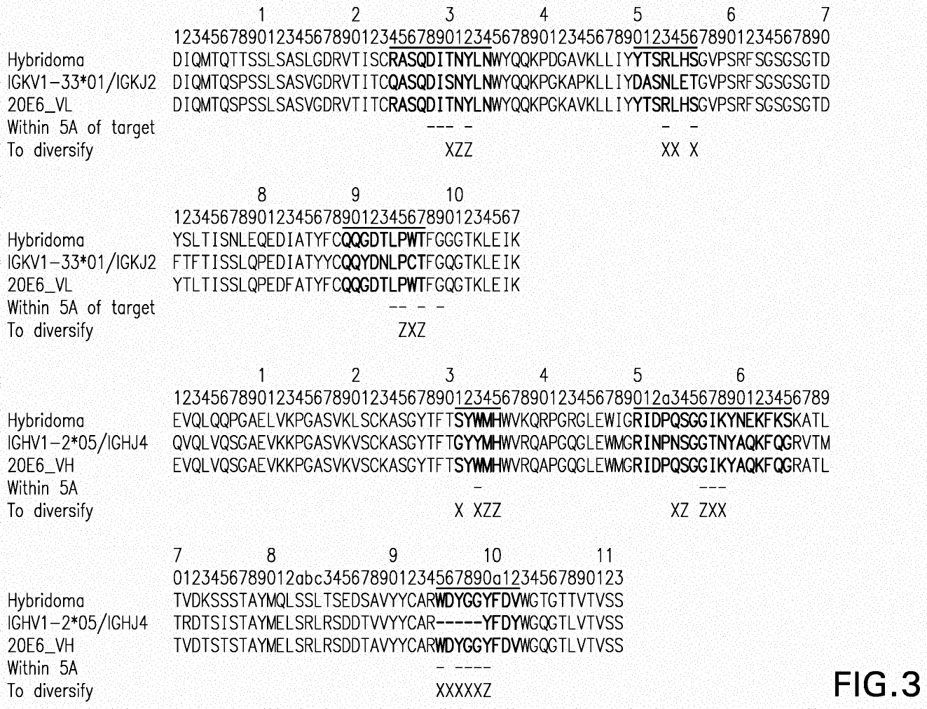

In a second approach, the TRIM nucleotide synthesis technology was utilized to diversify the light chain at residues T30, Y50, T51, R53, T93, and the heavy chain at residues S31, W33, Q53, 157, K58, W95, D96, Y97, G98, G99 (Kabat numbering). These residues of the 20E6 antibody encompass CDR positions that differ from the human germline IGKV1-33*01 or IGHV1-2*05 sequence. By synthesizing residues at these CDR positions as 49% of the parental codon and 51% of a mixture of 17 amino acid codons at 3% each (excluding methionine, cysteine, tryptophan), investigators were able to avoid undesirable mutations and stop codons while sampling all diversities more evenly. Additionally, the 79:7:7:7 doping method was used to diversify light chain residues N31, Y32, D92, L94, and heavy chain residues M34, H35, S54, G56, Y100 (Kabat numbering). These positions have the same residue between 20E6 antibody and the IGKV1-33*01 molecule or the IGHV1-2*05 molecule, and it was understood that the doping approach would better reflect the human antibody sequence diversity and frequency distribution at these positions (FIG. 3). Three ‘TRIM’ libraries for antibody 20E6 were constructed by routine recombinant DNA technologies containing CDR diversities in HCDR1 and 2, in HCDR3, and in all three LCDRs, respectively.

The doping 20E6 variant libraries and the TRIM 20E6 variant libraries were transformed into yeast cells by electroporation and displayed on cell surfaces as Fab fragments. Yeast libraries were generated by high-efficiency transformation of a genetically modified version of the BJ5465 strain (ATCC). Cells were grown to an optical density (OD) 600 nm of 1.6, spun down and washed 2× with water (or, in certain cases, 1 M sorbitol+1 mM CaCl₂) and 1× with electroporation buffer (1 M sorbitol+1 mM CaCl₂). Cells were then incubated in pre-treatment buffer (0.1 M LiAc+2.5 mM tris(2-carboxyethyl)phosphine (TCEP)) with shaking for 30 minutes at 30° C. Next, cells were spun down, washed 2× with cold electroporation buffer, and re-suspended in electroporation buffer to a final concentration of 2×10⁹ cells/mL. An amount (4 μg) of linearized vector and of DNA insert (12 μg) were added to 400 μL cells per cuvette. Electroporation using the exponential decay protocol was performed with a 2 mm cuvette with the following parameters: 2.6 kV, 200Ω resistance, 25 micro farad (ρF) capacitance, typically resulting in a time constant of 4.0 milliseconds (ms). After electroporation, recovery media (equal parts yeast extract-peptone-dextrose (YPD) media and 1 M sorbitol) was added and cells were incubated shaking for one hour at 30° C. Cells were then spun down and re-suspended in 1 M sorbitol at dilutions of 10⁻⁶, 10⁻⁷, and 10⁻⁸, and plated on glucose dropout media lacking leucine or tryptophan. Colonies were counted after three days growth to measure number of transformants.

### Example 8: Isolation of Affinity Matured 20E6 Variants by Yeast Surface Display

The yeast libraries were selected against a low concentration of biotinylated human and/or cyno LAP-TGFβ1-Fc fusion protein by magnetic-then fluorescence-activated cell sorting. The yeast libraries were exposed to different biotinylated LAP-TGFβ1-Fc antigen concentrations under different time and temperature conditions.

Fab expression was monitored by a goat F(ab′)2 anti-human kappa-Alexa Fluor 647 antibody (Southern Biotech). Antigen binding was monitored by R-Phycoerythrin conjugated Neutravidin (Sigma) as detection reagent. All samples were analyzed by flow cytometry using a FACS Aria III cytometer and FACS Diva software.

The optimal Ag binding conditions for library selections were identified by scouting experiments looked for conditions that appropriately discriminated library populations with different binding affinities towards LAP-TGFβ1-Fc. These conditions allowed detection of bound Ag and anti-human kappa expression to normalize the antigen-binding signal for expression. The libraries were sorted and collected for further rounds of analysis and enrichment. In the early rounds of selection, clones with the best antigen binding and expression in equilibrium conditions were collected. The antigen concentration was reduced in each successive round of selection and enrichment.

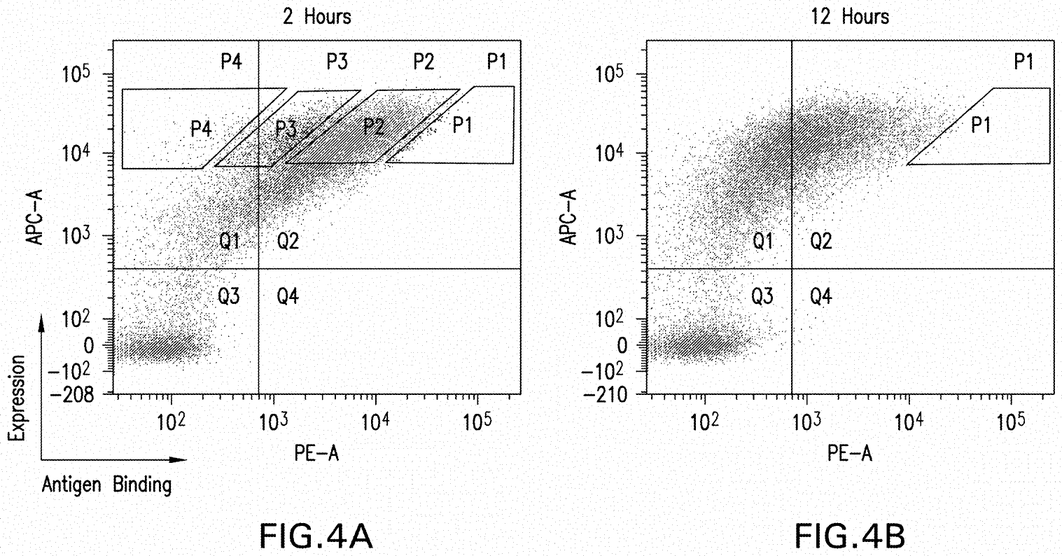

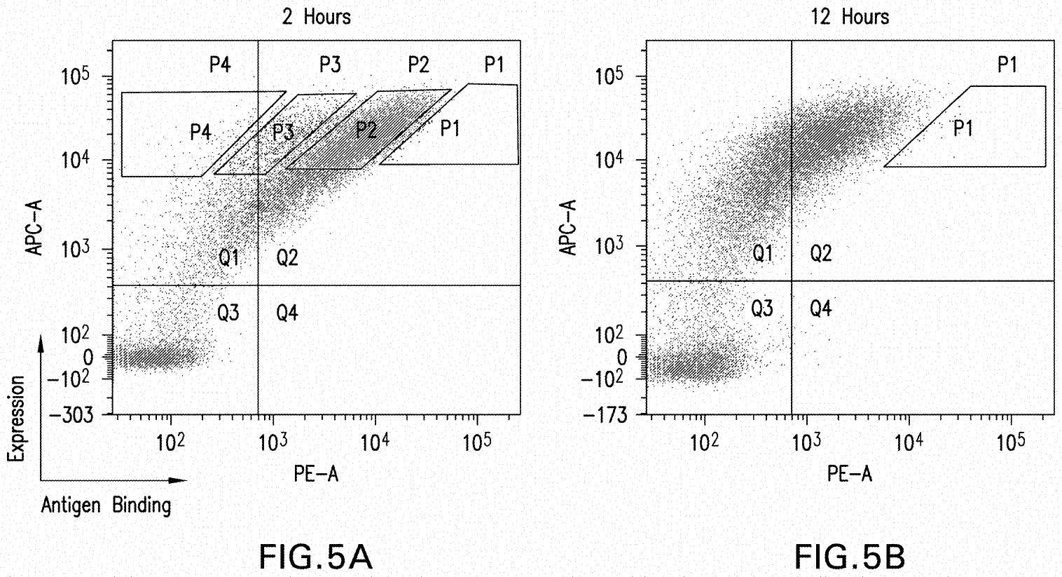

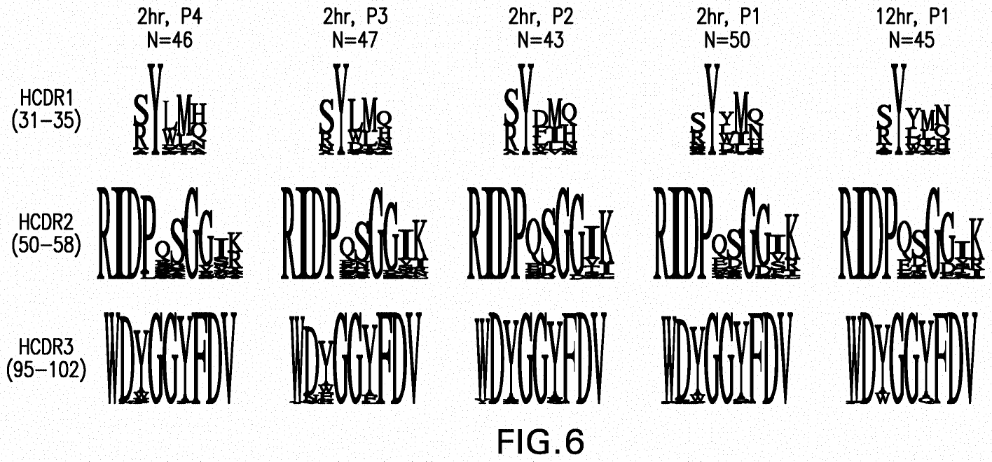

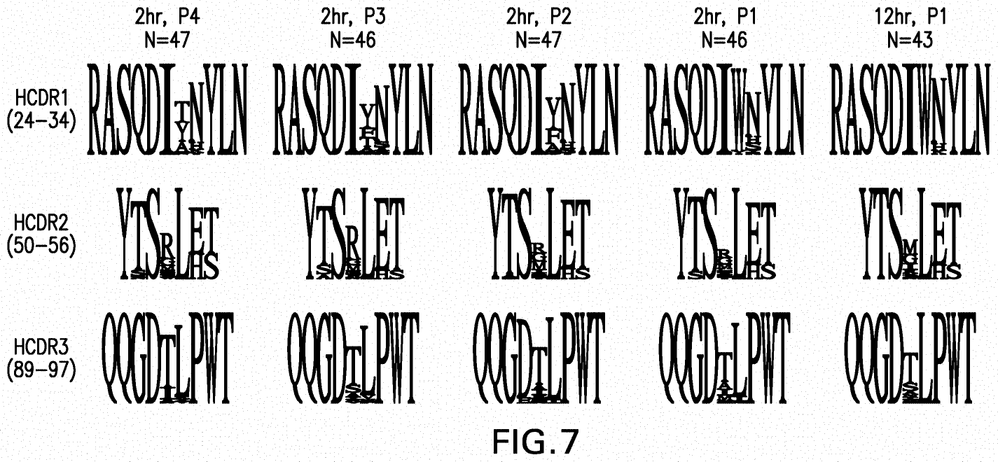

The libraries were last sorted by a kinetic selection for improved off-rate. Yeast cells displaying mutant 20E6 Fab were incubated in 300 pM biotinylated LAP-TGFβ1-Fc at 30° C. Cells were washed with phosphate buffered saline (PBS)-BSA buffer solution and then resuspended in a 100-fold excess of unbiotinylated LAP-TGFβ1-Fc protein and returned to 30° C. A collection of 20E6 variants with different affinity improvement were first sorted after 2 hours off-rate competition using 4 different sorting gates (P1 to P4 gates). See FIG. 4A and FIG. 5A. The highest affinity variant was sorted after 12 hours off-rate competition (compare FIG. 4A, FIG. 4B, FIG. 5A and FIG. 511).

### Example 9: Analysis of Affinity Matured 20E6 Variant Sequences from Different Off-Rate Selection Pressure

Yeast library outputs isolated from four 2-hour off-rate competition sorting gates and a 12-hour competition sorting gate were plated onto Petri dishes to form single colonies. Fifty randomly selected affinity matured yeast clones from each yeast library outputs were picked and their VH region and VL region were amplified by PCR for Sanger sequencing analysis. Individual 20E6 variant sequences from each output are listed in the following Tables 11-30 as indexed in Table 10. Tables 31-40 describe the consensus sequences of 20E6 variants from each selection output as well as amino acids that are represented at more than 3% of the available sequences at each CDR residue positions. It is expected that each identified amino acid change in the 20E6 CDRs alone or in combinations contribute to improved affinity to human LAP-TGFb1.

TABLE 10
Index of individual and consensus 20E6 variant VH and VL
sequences identified from the doping and TRIM libraries by
different sorting gates after 2 or 12 hours of off-rate competition

| | Off-rate | Doping library | | Trim library | |
|---|---|---|---|---|---|
| Sorting | duration | Sampled VH | Sampled VL | Sampled VH | Sampled VL |
| gate | competition | sequences | sequences | sequences | sequences |
| P1 | 2 hours | Tables 11, 31 | Tables 12, 31 | Tables 21, 36 | Tables 22, 36 |
| P2 | 2 hours | Tables 13, 32 | Tables 14, 32 | Tables 23, 37 | Tables 24, 37 |
| P3 | 2 hours | Tables 15, 33 | Tables 16, 33 | Tables 25, 38 | Tables 26, 38 |
| P4 | 2 hours | Tables 17, 34 | Tables 18, 34 | Tables 27, 39 | Tables 28, 39 |
| P1 | 12 hours | Tables 19, 35 | Tables 20, 35 | Tables 29, 40 | Tables 30, 40 |

TABLE 11
20E6 VH variants isolated from Doping Library
after 2 hour off-rate selection, P1 population

| Seq | |
|---|---|
| ID | |
| NO. | Amino acid sequence |
| 575 | EVQLVQSGAEVKKPGASVKVSCKASGYTFTNYYINWVRQAPGQGLEWMGRIDPQGGGSKYAQK |
| | FQGRATLTVDTSTSTAYMELSRLRSDDTAVYYCARWDYGGYFDVWGQGTLVTVSS |
| 576 | EVQLVQSGAEVKKPGASVKVSCKASGYTFTIYDTHWVRQAPGQGLEWMGRIDPQSGGIKYAQK |
| | FQGRATLTVDTSTSTAYMELSRLRSDDTAVYYCARWDYGGYFDVWGQGTLVTVSS |
| 577 | EVQLVQSGAEVKKPGASVKVSCKASGYTFTNYDIHWVRQAPGQGLEWMGRIDPQDGGIKYAQK |
| | FQGRATLTVDTSTSTAYMELSRLRSDDTAVYYCARWDYGGFFDVWGQGTLVTVSS |
| 578 | EVQLVQSGAEVKKPGASVKVSCKASGYTFTSYYLNWVRQAPGQGLEWMGRIDPWSGGIRYAQK |
| | FQGRATLTVDTSTSTAYMELSRLRSDDTAVYYCARWDYGGYFDVWGQGTLVTVSS |
| 579 | EVQLVQSGAEVKKPGASVKVSCKASGYTFTSYFMNWVRQAPGQGLEWMGRIDPQSGGSKYAQK |
| | FQGRATLTVDTSTSTAYMELSRLRSDDTAVYYCARWDYGGYFDVWGQGTLVTVSS |
| 580 | EVQLVQSGAEVKKPGASVKVSCKASGYTFTRYWISWVRQAPGQGLEWMGRIDPQSGGSRYAQK |
| | FQGRATLTVDTSTSTAYMELSRLRSDDTAVYYCARWDYGGYFDVWGQGTLVTVSS |
| 581 | EVQLVQSGAEVKKPGASVKVSCKASGYTFTSYLMQWVRQAPGQGLEWMGRIDPQSGGVKYAQK |
| | FQGRATLTVDTSTSTAYMELSRLRSDDTAVYYCARWDYGGYFDVWGQGTLVTVSS |
| 582 | EVQLVQSGAEVKKPGASVKVSCKASGYTFTSYWMAWVRQAPGQGLEWMGRIDPSDGGDRYAQK |
| | FQGRATLTVDTSTSTAYMELSRLRSDDTAVYYCARWDYGGYFDVWGQGTLVTVSS |
| 583 | EVQLVQSGAEVKKPGASVKVSCKASGYTFTSYWMQWVRQAPGQGLEWMGRIDPQDGGSKYAQK |
| | FQGRATLTVDTSTSTAYMELSRLRSDDTAVYYCARWDYGGYFDVWGQGTLVTVSS |
| 584 | EVQLVQSGAEVKKPGASVKVSCKASGYTFTSYFMNWVRQAPGQGLEWMGRIDPDRGGSKYAQK |
| | FQGRATLTVDTSTSTAYMELSRLRSDDTAVYYCARWHEGGYFDVWGQGTLVTVSS |
| 585 | EVQLVQSGAEVKKPGASVKVSCKASGYTFTSYLMQWVRQAPGQGLEWMGRIDPQLGDIKYAQK |
| | FQGRATLTMDTSTSTAYMELSRLRSDDTAVYYCARWDYGGYFDVWGQGTLVTVSS |
| 586 | EVQLVQSGAEVKKPGASVKVSCKASGYTFTSYWIQWVRQAPGQGLEWMGRIDPQSGGSKYAQK |
| | FQGRATLTVDTSTSTAYMELSRLRSDDTAVYYCARWDYGGYFDVWGQGTLVTVSS |
| 587 | EVQLVQSGAEVKKPGASVKVSCKASGYTFTRYYMNWVRQAPGQGLEWMGRIDPQSGGVKYAQK |
| | FQGRATLTVDTSTSTAYMELSRLRSDDTAVYYCARWDYGGYFDVWGQGTLVTVSS |
| 588 | EVQLVQSGAEVKKPGASVKVSCKASGYTFTRYLMQWVRQAPGQGLEWMGRIDPESGGVKYAQK |
| | FQGRATLTVDTSTSTAYMELSRLRSDDTAVYYCARWDYGGYFDVWGQGTLVTVSS |
| 589 | EVQLVQSGAEVKKPGASVKVSCKASGYTFTMYWMQWVRQAPGQGLEWMGRIDPISGGSKYAQK |
| | FQGRATLTVDTSTSTAYMELSRLRSDDTAVYYCARWDYGGYFDVWGQGTLVTVSS |
| 590 | EVQLVQSGAEVKKPGASVKVSCKASGYTFTSYWMQWVRQAPGQGLEWMGRIDPQNGGFKYAQK |
| | FQGRATLTVDTSTSTAYMELSRLRSDDTAVYYCARWDYGGYFDVWGQGTLVTVSS |
| 591 | EVQLVQSGAEVKKPGASVKVSCKASGYTFTNYYMAWVRQAPGQGLEWMGRIDPTSGGVKYAQK |
| | FQGRATLTVDTSTSTAYMELSRLRSDDTAVYYCARWDYGGYFDVWGHGTLVTVSS |
| 592 | EVQLVQSGAEVKKPGASVKVSCKASGYTFTRYYIQWVRQAPGQGLEWMGRIDPQDGDIKYAQK |
| | FQGRATLTVDTSTSTAYMELSRLRSDDTAVYYCARWDWGGYFDVWGQGTLVTVSS |
| 593 | EVQLVQSGAEVKKPGASVKVSCKASGYTFTSYLMGWVRQAPGQGLEWMGRIDPQSGGFKYAQK |
| | FQGRATLTVDTSTSTAYMELSRLRSDDTAVYYCARWEWGGQFDVWGQGTLVTVSS |
| 594 | EVQLVQSGAEVKKPGASVKVSCKASGYTFTRYLMHWVRQAPGQGLEWMGRIDPQNGGIRYAQK |
| | FQGRATLTVDTSTSTAYMELSRLRSDDTAVYYCARWDYGGYFDVWGQGTLVTVSS |
| 595 | EVQLVQSGAEVKKPGASVKVSCKASGYTFTSYYLNWVRQAPGQGLEWMGRIDPLGGGIRYAQK |
| | FQGRATLTVDTSTSTAYMELSRLRSDDTAVYYCARWDYGGYFDVWGQGTLVTVSS |
| 596 | EVQLVQSGAEVKKPGASVKVSCKASGYTFTTYWINWVRQAPGQGLEWMGRIDPLSGGVKYAQK |
| | FQGRATLTVDTSTSTAYMELSRLRSDDTAVYYCARWDYGGYFDVWGQGTLVTVSS |
| 597 | EVQLVQSGAEVKKPGASVKVSCKASGYTFTNYDMHWVRQAPGQGLEWMGRIDPQRGGILYAQK |
| | FQGRATLTVDTSTSTAYMELSRLRSDDTAVYYCARWDYGGYFDVWGQGTLVTVSS |
| 598 | EVQLVQSGAEVKKPGASVKVSCKASGYTFTTYDMHWVRQAPGQGLEWMGRIDPQAGDSIYAQK |
| | FQGRATLTVDTSTSTAYMELSRLRSDDTAVYYCARWDYGGYFDVWGQGTLVTVSS |
| 599 | EVQLVQSGAEVKKPGASVKVSCKASGYTFTRYYMNWVRQAPGQGLEWMGRIDPESGRFIYAQK |
| | FQGRATLTVDTSTSTAYMELSRLRSDDTAVYYCARWDYGGYFDVWGQGTLVTVSS |
| 600 | EVQLVQSGAEVKKPGASVKVSCKASGYTFTSYYLNWVRQAPGQGLEWMGRIDPQSGGSKYAQK |
| | FQGRATLTVDTSTSTAYMELSRLRSDDTAVYYCARWDYGGYFDVWGQGTLVTVSS |
| 601 | EVQLVQSGAEVKKPGASVKVSCKASGYTFTSYYMCWVRQAPGQGLEWMGRIDPQSGGVKYAQK |
| | FQGRATLTVDTSTSTAYMELSRLRSDDTAVYYCARWDYGGYFDVWGQGTLVTVSS |
| 602 | EVQLVQSGAEVKKPGASVKVSCKASGYTFTRYLMNWVRQAPGQGLEWMGRIDPEDGGCKYAQK |
| | FQGRATLTVDTSTSTAYMELSRLRSDDTAVYYCARWDYGGYFDVWGQGTLVTVSS |
| 603 | EVQLVQSGAEVKKPGASVKVSCKASGYTFTKYLIQWVRQAPGQGLEWMGRIDPEDGGIIYAQK |
| | FQGRATLTVDTSTSTAYMELSRLRSDDTAVYYCARWDYGGQFDVWGQGTLVTVSS |
| 604 | EVQLVQSGAEVKKPGASVKVSCKASGYTFTSYCMNWVRQAPGQGLEWMGRIDPHSGGVKYAQK |
| | FQGRATLTVDTSTSTAYMELSRLRSDDTAVYYCARWDWGGYFDVWGQGTLVTVSS |
| 605 | EVQLVQSGAEVKKPGASVKVSCKASGYTFTRYYMQWVRQAPGQGLEWMGRIDPESGGAKYAQK |
| | FQGRATLTVDTSTSTAYMELSRLRSDDTAVYYCARWDYGGYFDVWGQGTLVTVSS |
| 606 | EVQLVQSGAEVKKPGASVKVSCKASGYTFTHYYMQWVRQAPGQGLEWMGRIDPPSGDVRYAQK |
| | FQGRATLTVDTSTSTAYMELSRLRSDDTAVYYCARWDWGGYFDVWGQGTLVTVSS |
| 607 | EVQLVQSGAEVKKPGASVKVSCKASGYTFTSYWMQWVRQAPGQGLEWMGRIDPLIGDFKYAQK |
| | FQGRATLTVDTSTSTAYMELSRLRSDDTAVYYCARWDYGGYFDVWGQGTLVTVSS |
| 608 | EVOLVQSGAEVKKPGASVKVSCKASGYTFTRYFMNWVRQAPGQGLEWMGRIDPQEGGIKYAQK |
| | FQGRATLTVDTSTSTAYMELSRLRSDDTAVYYCARWDYGGYFDVWGQGTLVTVSS |
| 609 | EVQLVQSGAEVKKPGASVKVSCKASGYTFTSYDMHWVRQAPGQGLEWMGRIDPQSGGIKYAQK |
| | FQGRATLTVDTSTSTAYMELSRLRSDDTAVYYCARWDWGGYFDVWGQGTLVTVSS |
| 610 | EVQLVQSGAEVKKPGASVKVSCKASGYTFTFYDMHWVRQAPGQGLEWMGRIDPQSGGVKYAQK |
| | FQGRATLTVDTSTSTAYMELSRLRSDDTAVYYCARWDYGGYFDVWGQGTLVTVSS |
| 611 | EVQLVQSGAEVKKPGASVKVSCKASGYTFTRYWLQWVRQAPGQGLEWMGRIDPQIGEFKYAQK |
| | FQGRATLTVDTSTSTAYMELSRLRSDDTAVYYCARWDYGGYFDVWGQGTLVTVSS |
| 612 | EVQLVQSGAEVKKPGASVKVSCKASGYTFTNYDMHWVRQAPGQGLEWMGRIDPESGGIKYAQK |
| | FQGRATLTVDTSTSTAYMELSRLRSDDTAVYYCARWDYGGYFDVWGQGTLVTVSS |
| 613 | EVQLVQSGAEVKKPGASVKVSCKASGYTFTSYWMQWVRQAPGQGLEWMGRIDPQSGGNRYAQK |
| | FQGRATLTVDTSTSTAYVELSRLRSDDTAVYYCARWDYGGYFDVWGQGTLVTVSS |
| 614 | EVQLVQSGAEVKKPGASVKVSCKASGYTFTSYDMHWVRQAPGQGLEWMGRIDPQSGGVKYAQK |
| | FQGRATLTVDTSTSTAYMELSRLRSDDTAVYYCARWDYGGYFDVWGQGTLVTVSS |
| 615 | EVQLVQSGAEVKKPGASVKVSCKASGYTFTGYLMQWVRQAPGQGLEWMGRIDPQSGGIKYAQK |
| | FQGRATLTVDTSTSTAYMELSRLRSDDTAVYYCARWDYGGYFDVWGQGTLVTVSS |
| 616 | EVQLVQSGAEVKKPGASVKVSCKASGYTFTAYDIHWVRQAPGQGLEWMGRIDPQSGGIKYAQK |
| | FQGRATLTVDTSTSTAYMELSRLRSDDTAVYYCARWDYGGYFDVWGQGTLVTVSS |
| 617 | EVQLVQSGAEVKKPGASVKVSCKASGYTFTSYLMQWVRQAPGQGLEWMGRIDPDSGGILYAQK |
| | FQGRATLTVDTSTSTAYMELSRLRSDDTAVYYCARWDYGGYFDVWGQGTLVTVSS |
| 618 | EVQLVQSGAEVKKPGASVKVSCKASGYTFTSYYINWVRQAPGQGLEWMGRIDPDSGGFKYAQK |
| | FQGRATLTVDTSTSTAYMELSRLRSDDTAVYYCARWDYGGYFDVWGQGTLVTVSS |
| 619 | EVQLVQSGAEVKKPGASVKVSCKASGYTFTRYWMQWVRQAPGQGLEWMGRIDPQDGGIKYAQK |
| | FQGRATLTVDTSTSTAYMELSRLRSDDTAVYYCARFDYGGYFDVWGQGTLVTVSS |
| 620 | EVQLVQSGAEVKKPGASVKVSCKASGYTFTSYLLHWVRQAPGQGLEWMGRIDPEDGGIIYAQK |
| | FQGRATLTVDTSTSTAYMELSRLRSDDTAVYYCARWDYGGYFDVWGQGTLVTVSS |
| 621 | EVQLVQSGAEVKKPGASVKVSCKASGYTFTRYLMNWVRQAPGQGLEWMGRIDPLDGDIKYAQK |
| | FQGRATLTVDTSTSTAYMELSRLRSDDTAVYYCARWDYGGYFDVWGQGTLVTVSS |
| 622 | EVQLVQSGAEVKKPGASVKVSCKASGYTETRYYMSWVRQAPGQGLEWMGRIDPESGGIKYAQK |
| | FQGRATLTVDTSTSTAYMELSRLRSDDTAVYYCARWDYGGYFDVWGQGTLVTVSS |

TABLE 12
20E6 VL variants isolated from Doping Library after 2 hour off-rate selection,
P1 population

| Seq | |
|---|---|
| ID | |
| NO. | Amino acid sequence |
| 623 | DIQMTQSPSSLSASVGDRVTITCRASQDIWYYLNWYQQKPGKAVKLLIYYTSRLETGVPSRFS |
| | GSGSGTDYTLTISSLQPEDFATYFCQQGDTLPWTFGQGTKLEIK |
| 624 | DIQMTQSPSSLSASVGDRVTITCRASQDIWNYLNWYQQKPGKAVKLLIYYTSLLHSGVPSRFS |
| | GSGSGTDYTLTISSLQPEDFATYFCQQGDALPWTFGQGTKLEIK |
| 625 | DIQMTQSPSSLSASVGDRVTITCRASQDIWNYLNWYQQKPGKAVKLLIYYTSSLETGVPSRFS |
| | GSGSGTDYTLTISSLQPEDFATYFCQQGDTLPWTFGQGTKLEIK |
| 626 | DIQMTQSPSSLSASVGDRVTITCRASQDIWHYLNWYQQKPGKAVKLLIYYTSMLETGVPSRFS |
| | GSGSGTDYTLTISSLOPEDFATYFCQQGDTIPWTFGQGTKLEIK |
| 627 | DIQMTQSPSSLSASVGDRVTITCRASQDIWNYLNWYQQKPGKAVKLLIYYSSMLETGVPSRFS |
| | GSGSGTDYTLTISSLQPEDFATYFCQQGDILPWTFGQGTKLEIK |
| 628 | DIQMTQSPSSLSASVGDRVTITCRASQDIWHYLNWYQQKPGKAVKLLIYYTSCLETGVPSRFS |
| | GSGSGTDYTLTISSLOPEDFATYFCQQGDTLPWTFGQGTKLEIK |
| 629 | DIQMTQSPSSLSASVGDRVTITCRASQDIWNYLNWYQQKPGKAVKLLIYYTSRLETGVPSRFS |
| | GSGSGTDYTLTISSLQPEDFATYFCQQGDILPWTFGQGTELEIK |
| 630 | DIQMTQSPSSLSASVGDRVTITCRASQDIWNYLNWYQQKPGKAVKLLIYYTSRLHSGVPSRFS |
| | GSGSGTDYTLTISSLQPEDFATYFCQQGDTLPWTFGQGTKLEIK |
| 631 | DIQMTQSPSSLSASVGDRVTITCRASQDIWNYLNWYQQKPGKAVKLLIYYTSGLETGVPSRFS |
| | GSGSGTDYTLTISSLQPEDFATYFCQQGDTIPWTFGQGTKLEIK |
| 632 | DIQMTQSPSSLSASVGDRVTITCRASQDIWHYLNWYQQKPGKAVKLLIYYTSRLETGVPSRFS |
| | GSGSGTDYTLTISSLQPEDFATYFCQQGDILPWTFGQGTKLEIK |
| 633 | DIQMTQSPSSLSASVGDRVTITCRASQDIWYYLNWYQQKPGKAVKLLIYYTSGLETGVPSRFS |
| | GSGSGTDYTLTISSLQPEDFATYFCQQGDTLPWTFGQGTKLEIK |
| 634 | DIQMTQSPSSLSASVGDRVTITCRASQDIWNYLNWYQQKPGKAVKLLIYYTSRLETGVPSRFS |
| | GSGSGTDYTLTISSLQPEDFATYFCQQGDALPWTFGQGTKLEIK |
| 635 | DIQMTQSPSSLSASVGDRVTITCRASQDIWNYLNWYQQKPGKAVKLLIYYTSGLETGVPSRFS |
| | GSGSGTDYTLTISSLQPEDFATYFCQQGDTLPWTFGQGTKLEIK |
| 636 | DIQMTQSPSSLSASVGDRVTITCRASQDIYKYLNWYQQKPGKAVKLLIYYTSGLETGVPSRFS |
| | GSGSGTDYTLTISSLQPEDFATYFCQQGDTLPWTFGQGTKLEIK |
| 637 | DIQMTQSPSSLSASVGDRVTITCRASQDIWNYLNWYQQKPGKAVKLLIYYTSGLETGVPSRFS |
| | GSGSGTDYTLTISSLQPEDFATYFCQQGDNLPWTFGQGTKLEIK |
| 638 | DIQMTQSPSSLSASVGDRVTITCRASQDIWNYLNWYQQKPGKAVKLLIYYSSVLETGVPSRFS |
| | GSGSGTDYTLTISSLQPEDFATYFCQQGDTLPWTFGQGTKLEIK |
| 639 | DIQMTQSPSSLSASVGDRVTITCRASQDIWNYLNWYQQKPGKAVKLLIYYTSNLETGVPSRFS |
| | GSGSGTDYTLTISSLQPEDFATYFCQQGDTLPWTFGQGTKLEIK |
| 640 | DIQMTQSPSSLSASVGDRVTITCRASQDIWSYLNWYQQKPGKAVKLLIYYTSGLETGVPSRFS |
| | GSGSGTDYTLTISSLQPEDFATYFCQQGDTLPWTFGQGTKLEIK |
| 641 | DIQMTQSPSSLSASVGDRVTITCRASQDIWNYLNWYQQKPGKAVKLLIYYTSILETGVPSRFS |
| | GSGSGTDYTLTISSLQPEDFATYFCQQGDTLPWTFGQGTKLEIK |
| 642 | DIQMTQSPSSLSASVGDRVTITCRASQDIWNYLNWYQQKPGKAVKLLIYYTSMLETGVPSRFS |
| | GSGSGTDYTLTISSLQPEDFATYFCQQGDTIPWTFGQGTKLEIK |
| 643 | DIQMTQSPSSLSASVGDRVTITCRASQDIWNYLNWYQQKPGKAVKLLIYYTSSLETGVPSRFS |
| | GSGSGTDYTLTISSLQPEDFATYFCQQGDILPWTFGQGTKLEIK |
| 644 | DIQMTQSPSSLSASVGDRVTITCRASQDIWNYLNWYQQKPGKAVKLLIYYTSRLETGVPSRFS |
| | GSGSGTDYTLTISSLQPEDFATYFCQQGDTLPWTFGQGTKLEIK |
| 645 | DIQMTQSPSSLSASVGDRVTITCRASQDIWSYLNWYQQKPGKAVKLLIYYTSRLHSGVPSRFS |
| | GSGSGTDYTLTISSLQPEDFATYFCQQGDALPWTFGQGTKLEIK |
| 646 | DIQMTQSPSSLSASVGDRVTITCRASQDIWNYLNWYQQKPGKAVKLLIYYTSRLETGVPSRFS |
| | GSGSGTDYTLTISSLOPEDFATYFCQQGDLLPWTFGQGTKLEIK |
| 647 | DIQMTQSPSSLSASVGDRVTITCRASQDIYNYLNWYQQKPGKAVKLLIYYTSRLETGVPSRFS |
| | GSGSGTDYTLTISSLQPEDFATYFCQQGDMLPWTFGQGTKLEIK |
| 648 | DIQMTQSPSSLSASVGDRVTITCRASQDIWNYLNWYQQKPGKAVKLLIYYTSSLETGVPSRFS |
| | GSGSGTDYTLTISSLQPEDFATYFCQQGDIMPWTFGQGTKLEIK |
| 649 | DIQMTQSPSSLSASVGDRVTITCRASQDIWNYLNWYQQKPGKAVKLLIYYTSTLETGVPSRFS |
| | GSGSGTDYTLTISSLQPEDFATYFCQQGDMLPWTFGQGTKLEIK |
| 650 | DIQMTQSPSSLSASVGDRVTITCRASQDIWSYLNWYQQKPGKAVKLLIYYTSGLHSGVPSRFS |
| | GSGSGTDYTLTISSLQPEDFATYFCQQGDALPWTFGQGTKLEIK |
| 651 | DIQMTQSPSSLSASVGDRVTITCRASQDIWNYLNWYQQKPGKAVKLLIYYTSILHSGVPSRFS |
| | GSGSGTDYTLTISSLQPEDFATYFCQQGDTLPWTFGQGTKLEIK |
| 652 | DIQMTQSPSSLSASVGDRVTITCRASQDIWNYLNWYQQKPGKAVKLLIYYTSWLETGVPSRFS |
| | GSGSGTDYTLTISSLQPEDFATYFCQQGDTLPWTFGQGTKLEIK |
| 653 | DIQMTQSPSSLSASVGDRVTITCRASQDIWNYLNWYQQKPGKAVKLLIYYTSGLHSGVPSRFS |
| | GSGSGTDYTLTISSLQPEDFATYFCQQGDSLPWTFGQGTKLEIK |
| 654 | DIQMTQSPSSLSASVGDRVTITCRASQDIWNYLNWYQQKPGKAVKLLIYYTSLLHSGVPSRFS |
| | GSGSGTDYTLTISSLQPEDFATYFCQQGDTLPWTFGQGTKLEIK |
| 655 | DIQMTQSPSSLSASVGDRVTITCRASQDIWHYLNWYQQKPGKAVKLLIYYTSLLETGVPSRFS |
| | GSGSGTDYTLTISSLQPEDFATYFCQQGDTLPWTFGQGTKLEIK |
| 656 | DIQMTQSPSSLSASVGDRVTITCRASQDIWNYLNWYQQKPGKAVKLLIYYTSMLETGVPSRFS |
| | GSGSGTDYTLTISSLQPEDFATYFCQQGDMLPWTFGQGTKLEIK |
| 657 | DIQMTQSPSSLSASVGDRVTITCRASQDIWSYLNWYQQKPGKAVKLLIYYTSRLHSGVPSRFS |
| | GSGSGTDYTLTISSLQPEDFATYFCQQGDTLPWTFGQGTKLEIK |
| 658 | DIQMTQSPSSLSASVGDRVTITCRASQDIWNYLNWYQQKPGKAVKLLIYYTSGLETGVPSRFS |
| | GSGSGTDYTLTISSLOPEDFATYFCQQGDALPWTFGQGTKLEIK |
| 659 | DIQMTQSPSSLSASVGDRVTITCRASQDIWNYLNWYQQKPGKAVKLLIYYTSMLETGVPSRFS |
| | GSGSGTDYTLTISSLQPEDFATYFCQQGDTLPWTFGQGTKLEIK |
| 660 | DIQMTQSPSSLSASVGDRVTITCRASQDIWSYLNWYQQKPGKAVKLLIYYTSWLETGVPSRFS |
| | GSGSGTDYTLTISSLQPEDFATYFCQQGDTLPWTFGQGTKLEIK |

TABLE 13
20E6 VH variants isolated from Doping Library after 2 hour off-rate selection, P2
population

| Seq | |
|---|---|
| ID | |
| NO . | Amino acid sequence |
| 661 | EVQLVQSGAEVKKPGASVKVSCKASGYTFTRYDIHWVRQAPGQGLEWMGRIDPQSGGSKYAQK |
| | FQGRATLTVDTSTSTAYMELSRLRSDDTAVYYCARWDYGGYFDVWGQGTLVTVSS |
| 662 | EVQLVQSGAEVKKPGASVKVSCKASGYTFTRYDIHWVRQAPGQGLEWMGRIDPQSGGTKYAQK |
| | FQGRATLTVDTSTSTAYMELSRLRSDDTAVYYCARWDYGGYFDVWGQGTLVTVSS |
| 663 | EVQLVQSGAEVKKPGASVKVSCKASGYTFTSYSMHWVRQAPGQGLEWMGRIDPQDGDIKYAQK |
| | FQGRATLTVDTSTSTAYMELSRLRSDDTAVYYCARWDYGGYFDVWGQGTLVTVSS |
| 664 | EVQLVQSGAEVKKPGASVKVSCKASGYTFTSYDVHWVRQAPGQGLEWMGRIDPDGGDIKYAQK |
| | FQGRATLTVDTSTSTAYMELSRLRSDDTAVYYCARWDYGGYFDVWGQGTLVTVSS |
| 665 | EVQLVQSGAEVKKPGASVKVSCKASGYTFTSYLMAWVRQAPGQGLEWMGRIDPQSGGIKYAQK |
| | FQGRATLTVDTSTSTAYMELSRLRSDDTAVYYCARWDYGGQFDVWGQGTLVTVSS |
| 666 | EVQLVQSGAEVKKPGASVKVSCKASGYTFTSYYMNWVRQAPGQGLEWMGRIDPLSGGIKYAQK |
| | FQGRATLTVDTSTSTAYMELSRLRSDDTAVYYCARYDYGGFFDVWGQGTLVTVSS |
| 667 | EVQLVQSGAEVKKPGASVKVSCKASGYTFTSYDLHWVRQAPGQGLEWMGRIDPQSGGIKYAQK |
| | FQGRATLTVDTSTSTAYMELSRLRSDDTAVYYCARWDYGGYFDVWGQGTLVTVSS |
| 668 | EVQLVQSGAEVKKPGASVKVSCKASGYTFTSYWMQWVRQAPGQGLEWMGRIDPQSGGILYAQK |
| | FQGRATLTVDTSTSTAYMELSRLRSDDTAVYYCARWDYGGYFDVWGQGTLVTVSS |
| 669 | EVQLVQSGAEVKKPGASVKVSCKASGYTFTRYDLYWVRQAPGQGLEWMGRIDPEGGGVKYAQK |
| | FQGRATLTVDTSTSTAYMELSRLRSDDTAVYYCARWDYGGYFDVWGQGTLVTVSS |
| 670 | EVQLVQSGAEVKKPGASVKVSCKASGYTFTSYFMQWVRQAPGQGLEWMGRIDPQSGGIKYAQK |
| | FQGRATLTVDTSTSTAYMELSKLRSDDTAVYYCARWDYGGYFDVWGQGTLVTVSS |
| 671 | EVQLVQSGAEVKKPGASVKVSCKASGYTFTSYYISWVRQAPGQGLEWMGRIDPNSGGIKYAQK |
| | FQGRATLTVDTSTSTAYMELSRLRSDDTAVYYCARYDHGGYFDVWGQGTLVTVSS |
| 672 | EVQLVQSGAEVKKPGASVKVSCKASGYTFTSYFLNWVRQAPGQGLEWMGRIDPQDGGIKYAQK |
| | FQGRATLTVDTSTSTAYMELSRLRSDDTAVYYCARWDYGGYFDVWGQGTLVTVSS |
| 673 | EVQLVQSGAEVKKPGASVKVSCKASGYTFTRYLMQWVRQAPGQGLEWMGRIDPQSGGIRYAQK |
| | FQGRATLTVDTSTSTAYMELSRLRSDDTAVYYCARWDYGGYFDVWGQGTLVTVSS |
| 674 | EVQLVQSGAEVKKPGASVKVSCKASGYTFTAYDIHWVRQAPGQGLEWMGRIDPQSGGIKYAQK |
| | FQGRATLTVDTSTSTAYMELSRLRSDDTAVYYCARWDYGGYFDVWGQGTLVTVSS |
| 675 | EVQLVQSGAEVKKPGASVKVSCKASGYTFTSYYINWVRQAPGQGLEWMGRIDPDSGGFKYAQK |
| | FQGRATLTVDTSTSTAYMELSRLRSDDTAVYYCARWDYGGYFDVWGQGTLVTVSS |
| 676 | EVQLVQSGAEVKKPGASVKVSCKASGYTFTSYDLHWVRQAPGQGLEWMGRIDPQSGGILYAQK |
| | FQGRATLTVDTSTSTAYMELSRLRSDDTAVYYCARWDYGGFFDVWGQGTLVTVSS |
| 677 | EVQLVQSGAEVKKPGASVKVSCKASGYTFTRYDVHWVRQAPGQGLEWMGRIDPQSGGKKYAQK |
| | FQGRATLTVDTSTSTAYMELSRLRSDDTAVYYCARWDYGGYFDVWGQGTLVTVSS |
| 678 | EVQLVQSGAEVKKPGASVKVSCKASGYTFTSYLMQWVRQAPGQGLEWMGRIDPQSGGIIYAQK |
| | FQGRATLTVDTSTSTAYMELSRLRSDDTAVYYCARWDYGGYFDVWGQGTLVTVSS |
| 679 | EVQLVQSGAEVKKPGASVKVSCKASGYTFTRYLMQWVRQAPGQGLEWMGRIDPQSGGIIYAQK |
| | FQGRATLTVDTSTSTAYMELSRLRSDDTAVYYCARWDYGGYFDVWGQGTLVTVSS |
| 680 | EVQLVQSGAEVKKPGASVKVSCKASGYTFTRYLMQWVRQAPGQGLEWMGRIDPQSGGVIYAQK |
| | FQGRATLTVDTSTSTAYMELSRLRSDDTAVYYCARWDFGGYFDVWGQGTLVTVSS |
| 681 | EVQLVQSGAEVKKPGASVKVSCKASGYTFTSYWMQWVRQAPGQGLEWMGRIDPPSGGVIYAQK |
| | FQGRATLTVDTSTSTAYMELSRLRSDDTAVYYCARWDYGGYFDVWGQGTLVTVSS |
| 682 | EVOLVQSGAEVKKPGASVKVSCKASGYTFTAYLMQWVRQAPGQGLEWMGRIDPEDGGVKYAQK |
| | FQGRATLTVDTSTSTAYMELSRLRSDDTAVYYCARWDYGGYFDVWGQGTLVTVSS |
| 683 | EVQLVQSGAEVKKPGASVKVSCKASGYTFTSYFMNWVRQAPGQGLEWMGRIDPQSGGIKYAQK |
| | FQGRATLTVDTSTSTAYMELSRLRSDDTAVYYCARWDYGGYFDVWGQGTLVTVSS |
| 684 | EVQLVQSGAEVKKPGASVKVSCKASGYTFTSYWIQWVRQAPGQGLEWMGRIDPEDGGIKYAQK |
| | FQGRATLTVDTSTSTAYMELSRLRSDDTAVYYCARWDYGGYFDVWGQGTLVTVSS |
| 685 | EVQLVQSGAEVKKPGASVKVSCKASGYTFTSYWMQWVRQAPGQGLEWMGRIDPHDGGIKYAQK |
| | FQGRATLTVDTSTSTAYMELSRLRSDDTAVYYCARWDYGGYFDVWGQGTLVTVSS |

TABLE 14
20E6 VL variants isolated from Doping Library after 2 hour off-rate selection,
P2 population

| Seq | |
|---|---|
| ID | |
| NO. | Amino acid sequence |
| 686 | DIQMTQSPSSLSASVGDRVTITCRASQDISNYLNWYQQKPGKAVKLLIYYTSMLETGVPSRFS |
| | GSGSGTDYTLTISSLQPEDFATYFCQQGDALPWTFGRGTKLEIK |
| 687 | DIQMTQSPSSLSASVGDRVTITCRASQDIYNYLNWYQQKPGKAVKLLIYYTSGLETGVPSRFS |
| | GSGSGTDYTLTISSLQPEDFATYFCQQGDTLPWTFGQGTKLEIK |
| 688 | DIQMTQSPSSLSASVGDRVTITCRASQDIYNYLNWYQQKPGKAVKLLIYYTSILETGVPSRFS |
| | GSGSGTDYTLTISSLQPEDFATYFCQQGDTLPWTFGQGTKLEIK |
| 689 | DIQMTQSPSSLSASVGDRVTITCRASQDIYNYLNWYQQKPGKAVKLLIYYTSRLETGVPSRFS |
| | GSGSGTDYTLTISSLQPEDFATYFCQQGDILPWTFGQGTKLEIK |
| 690 | DIQMTQSPSSLSASVGDRVTITCRASQDIFNYLNWYQQKPGKAVKLLIYYASTLHSGVPSRFS |
| | GSGSGTDYTLTISSLQPEDFATYFCQQGDTLPWTFGQGTKLEIK |
| 691 | DIQMTQSPSSLSASVGDRVTITCRASQDIYSYLNWYQQKPGKAVKLLIYYTSRLHSGVPSRFS |
| | GSGSGTDYTLTISSLQPEDFATYFCQQGDQLPWTFGQGTKLEIK |
| 692 | DIQMTQSPSSLSASVGDRVTITCRASQDIYNYLNWYQQKPGKAVKLLIYYTSKLHSGVPSRFS |
| | GSGSGTDYTLTISSLQPEDFATYFCQQGDILPWTFGQGTKLEIK |
| 693 | DIQMTQSPSSLSASVGDRVTITCRASQDIYNYLNWYQQKPGKAVKLLIYYTSQLETGVPSRFS |
| | GSGSGTDYTLTISSLQPEDFATYFCQQGDTLPWTFGQGTKLEIK |
| 694 | DIQMTQSPSSLSASVGDRVTITCRASQDIYNYLNWYQQKPGKAVKLLIYYTSRLETGVPSRFS |
| | GSGSGTDYTLTISSLQPEDFATYFCQQGDTLPWTFGQGTKLEIK |
| 695 | DIQMTQSPSSLSASVGDRVTITCRASQDIFHYLNWYQQKPGKAVKLLIYYTSELETGVPSRFS |
| | GSGSGTDYTLTISSLQPEDFATYFCQQGDTLPWTFGQGTKLEIK |
| 696 | DIQMTQSPSSLSASVGDRVTITCRASQDIYNYLNWYQQKPGKAVKLLIYYTSRLETGVPSRFS |
| | GSGSGTDYTLTISSLQPEDFATYFCQQGDSIPWTFGQGTKLEIK |
| 697 | DIQMTQSPSSLSASVGDRVTITCRASQDIYNYLNWYQQKPGKAVKLLIYYTSGLHSGVPSRFS |
| | GSGSGTDYTLTISSLQPEDFATYFCQQGDALPWTFGQGTKLEIK |
| 698 | DIQMTQSPSSLSASVGDRVTITCRASQDIYYYLNWYQQKPGKAVKLLIYYTSGLETGVPSRFS |
| | GSGSGTDYTLTISSLQPEDFATYFCQQGDTLPWTFGQGTKLEIK |
| 699 | DIQMTQSPSSLSASVGDRVTITCRASQDIFNYLNWYQQKPGKAVKLLIYYTSSLETGVPSRFS |
| | GSGSGTDYTLTISSLQPEDFATYFCQQGDSLPWTFGQGTKLEIK |
| 700 | DIQMTQSPSSLSASVGDRVTITCRASQDISNYLNWYQQKPGKAVKLLIYYTSMLETGVPSRFS |
| | GSGSGTDYTLTISSLQPEDFATYFCQQGDILPWTFGQGTKLEIK |
| 701 | DIQMTQSPSSLSASVGDRVTITCRASQDIANYLNWYQQKPGKAVKLLIYYTSMLETGVPSRFS |
| | GSGSGTDYTLTISSLQPEDFATYFCQQGETLPWTFGQGTKLEIK |
| 702 | DIQMTQSPSSLSASVGDRVTITCRASQDIFSYLNWYQQKPGKAVKLLIYYTSILETGVPSRFS |
| | GSGSGTDYTLTISSLQPEDFATYFCQQGDTLPWTFGQGTKLEIK |
| 703 | DIQMTQSPSSLSASVGDRVTITCRASQDIFNYLNWYQQKPGKAVKLLIYYTSRLETGVPSRFS |
| | GSGSGTDYTLTISSLQPEDFATYFCQQGDTLPWTFGQGTKLEIK |
| 704 | DIQMTQSPSSLSASVGDRVTITCRASQDIFNYLNWYQQKPGKAVKLLIYYTSSLETGVPSRFS |
| | GSGSGTDYTLTISSLQPEDFATYFCQQGDTLPWTFGQGTKLEIK |
| 705 | DIQMTQSPSSLSASVGDRVTITCRASQDIFNYLNWYQQKPGKAVKLLIYYTSRLETGVPSRFS |
| | GSGSGTDYTLTISSLQPEDFATYFCQQGDELPWTFGQGTKLEIK |
| 706 | DIQMTQSPSSLSASVGDRVTITCRASQDIYNYLNWYQQKPGKAVKLLIYYTSMLETGVPSRFS |
| | GSGSGTDYTLTISSLQPEDFATYFCQQGDTLPWTFGQGTKLEIK |
| 707 | DIQMTQSPSSLSASVGDRVTITCRASQDIANYLNWYQQKPGKAVKLLIYYTSILETGVPSRFS |
| | GSGSGTDYTLTISSLQPEDFATYFCQQGDTLPWTFGQGTKLEIK |
| 708 | DIQMTQSPSSLSASVGDRVTITCRASQDIYHYLNWYQQKPGKAVKLLIYYTSRLETGVPSRFS |
| | GSGSGTDYTLTISSLOPEDFATYFCQQGDTLPWTFGQGTKLEIK |
| 709 | DIQMTQSPSSLSASVGDRVTITCRASQDIANYLNWYQQKPGKAVKLLIYYTSMLETGVPSRFS |
| | GSGSGTDYTLTISSLQPEDFATYFCQQGDTLPWTFGQGTKLEIK |
| 710 | DIQMTQSPSSLSASVGDRVTITCRASQDIYNYLNWYQQKPGKAVKLLIYYSSMLETGVPSRFS |
| | GSGSGTDYTLTISSLQPEDFATYFCQQGDELPWTFGQGTKLEIK |
| 711 | DIQMTQSPSSLSASVGDRVTITCRASQDIYNYLNWYQQKPGKAVKLLIYYTSRLETGVPSRFS |
| | GSGSGTDYTLTISSLQPEDFATYFCQQGDIIPWTFGQGTKLEIK |

TABLE 15
20E6 VH variants isolated from Doping Library after 2 hour off-rate selection,
P3 population

| Seq | |
|---|---|
| ID | |
| NO. | Amino acid sequence |
| 712 | EVQLVQSEAEVKKPGASVKVSCKASGYTFTSYQLQWVRQAPGQGLEWMGRIDPHGGSTNYAQK |
| | FQGRATLTVDTSTSTAYMELSRLRSDDTAVYYCARWDYGGYFDVWGQGTLVTVSS |
| 713 | EVQLVQSGAEVKKPGASVKVSCKASGYTFTSYDMCWVRQAPGQGLEWMGRIDPQSGGIRYAQK |
| | FQGRATLTVDTSTSTAYMELSRLRSDDTAVYYCARWDFGGYFDVWGQGTLVTVSS |
| 714 | EVOLVQSGAEVKKPGASVKVSCKASGYTFTSYWMQWVRQAPGQGLEWMGRIDPQSGGIKYAQK |
| | FQGRATLTVDTSTSTAYMELSRLRSDDTAVYYCARWDYGGYFDVWGQGTLVTVSS |
| 715 | EVQLVQSGAEVKKPGASVKVSCKASGYTFTSYLMHWVRQAPGQGLEWMGRIDPRNGGIPYAQK |
| | FQGRATLTVDTSTSTAYMELSRLRSDDTAVYYCARWDYGGYFDVWGQGTLVTVSS |
| 716 | EVQLVQSGAEVKKPGASVKVSCKASGYTFTSYLLQWVRQAPGQGLEWMGRIDPQSGGNKYAQK |
| | FQGRATLTVDTSTSTAYMELSRLRSDDTAVYYCARWDFGGYFDVWGQGTLVTVSS |
| 717 | EVQLVQSGAEVKKPGASVKVSCKASGYTFTSYLMHWVRQAPGQGLEWMGRIDPQSGGFIYAQK |
| | FQGRATLTVDTSTSTAYMELSRLRSDDTAVYYCARYDHGGYFDVWGQGTLVTVSS |
| 718 | EVQLVQSGAEVKKPGASVKVSCKASGYTFTRYLMNWVRQAPGQGLEWMGRIDPPSGGVKYAQK |
| | FQGRATLTVDTSTSTAYMELSRLRSDDTAVYYCARWDYGGYFDVWGQGTLVTVSS |
| 719 | EVQLVQSGAEVKKPGASVKVSCKASGYTFTSYLLSWVRQAPGQGLEWMGRIDPDSGGIKYAQK |
| | FQGRATLTVDTSTSTAYMELSRLRSDDTAVYYCARWDYGGFFDVWGQGTLVTVSS |
| 720 | EVQLVQSGAEVKKPGASVKVSCKASGYTFTKYLMTWVRQAPGQGLEWMGRIDPQSGGIKYAQK |
| | FQGRATLTVDTSTSTAYMELSRLRSDDTAVYYCARWDYGGYFDVWGQGTLVTVSS |
| 721 | EVQLVQSGAEVKKPGASVKVSCKASGYTFTIYDMHWVRQAPGQGLEWMGRIDPQLGGIKYAQK |
| | FQGRATLTVDTSTSTAYMELSRLRSDDTAVYYCARWQWGGEFDVWGQGTLVTVSS |
| 722 | EVQLVQSGAEVKKPGASVKVSCKASGYTFTSYLMHWVRQAPGQGLEWMGRIDPQSGGIIYAQK |
| | FQGRATLTVDTSTSTAYMELSRLRSDDTAVYYCARWHFGGYFDVWGQGTLVTVSS |
| 723 | EVQLVOSGAEVKKPGASVKVSCKASGYTFTQYLMSWVRQAPGQGLEWMGRIDPQSGGIKYAQK |
| | FQGRATLTVDTSTSTAYMELSRLRSDDTAVYYCARWDYGGYFDVWGQGTLVTVSS |
| 724 | EVQLVQSGAEVKKPGASVKVSCKASGYTFTRYLLQWVRQAPGQGLEWMGRIDPQGGGIKYAQK |
| | FQGRATLTVDTSTSTAYMELSRLRSDDTAVYYCARWDYGGYFDVWGQGTLVTVSS |
| 725 | EVQLVQSGAEVKKPGASVKVSCKASGYTFTSYWMHWVRQAPGQGLEWMGRIDPVGGGTKYAQK |
| | FQGRATLTVDTSTSTAYMELSRLRSDDTAVYYCARWNYGGYFDVWGQGTLVTVSS |
| 726 | EVOLVQSGAEVKKPGASVKVSCKASGYTFTRYWMSWVRQAPGQGLEWMGRIDPQSGGIIYAQK |
| | FQGRATLTVDTSTSTAYMELSRLRSDDTAVYYCARWDWGGYFDVWGQGTLVTVSS |
| 727 | EVQLVQSGAEVKKPGASVKVSCKASGYTFTSYLMQWVRQAPGQGLEWMGRIDPRSGGILYAQK |
| | FQGRATLTVDTSTSTAYMELSRLRSDDTAVYYCARWNYGGYFDVWGQGTLVTVSS |
| 728 | EVQLVQSGAEVKKPGASVKVSCKASGYTFTSYLNHWVRQAPGQGLEWMGRIDPESGGSRYAQK |
| | FQGRATLTVDTSTSTAYMELSRLRSDDTAVYYCARWDHGGYFDVWGQGTLVTVSS |
| 729 | EVQLVQSGAEVKKPGASVKVSCKASGYTFTSYLMQWVRQAPGQGLEWMGRIDPRDGGINYAQK |
| | FQGRATLTVDTSTSTAYMELSRLRSDDTAVYYCARWDYGGYFDVWGQGTLVTVSS |
| 730 | EVOLVQSGAEVKKPGASVKVSCKASGYTFTRYWMQWVRQAPGQGLEWMGRIDPLSGGMKYAQK |
| | FQGRATLTVDTSTSTAYMELSRLRSDDTAVYYCARWNHGGYFDVWGQGTLVTVSS |
| 731 | EVQLVQSGAEVKKPGASVKVSCKASGYTFTSYWIQWVRQAPGQGLEWMGRIDPQSGGSKYAQK |
| | FQGRATLTVDTSTSTAYMELSRLRSDDTAVYYCARWDYGGYFDVWGQGTLVTVSS |
| 732 | EVQLVQSGAEVKKPGASVKVSCKASGYTFTNYLLHWVRQAPGQGLEWMGRIDPQSGDIKYAQK |
| | FQGRATLTVDTSTSTAYMELSRLRSDDTAVYYCARWDWGGYFDVWGQGTLVTVSS |
| 733 | EVQLVQSGAEVKKPGASVKVSCKASGYTFTSYWMHWVRQAPGQGLEWMGRIDPKNGGIIYAQK |
| | FQGRATLTVDTSTSTAYMELSRLRSDDTAVYYCARWDWGGFFDVWGQGTLVTVSS |
| 734 | EVQLVQSGAEVKKPGASVKVSCKASGYTFTSYDTNWVRQAPGQGLEWMGRIDPQNGGIKYAQK |
| | FQGRATLTVDTSTSTAYMELSRLRSDDTAVYYCARWDYGGYFDVWGQGTLVTVSS |
| 735 | EVQLVQSGAEVKKPGASVKVSCKASGYTFTGYLVSWVRQAPGQGLEWMGRIDPRSGGIIYAQK |
| | FQGRATLTVDTSTSTAYMELSRLRSDDTAVYYCARWDYGGYFDVWGQGTLVTVSS |
| 736 | EVQLVQSGAEVKKPGASVKVSCKASGYTFTNYLMQWVRQAPGQGLEWMGRIDPQIGGSRYAQK |
| | FQGRATLTVDTSTSTAYMELSRLRSDDTAVYYCARWDYGGYFDVWGQGTLVTVSS |
| 737 | EVQLVQSGAEVKKPGASVKVSCKASGYTFTRYDLQWVRQAPGQGLEWMGRIDPPSGGINYAQK |
| | FQGRATLTVDTSTSTAYMELSRLRSDDTAVYYCARWDYGGYFDVWGQGTLVTVSS |
| 738 | EVQLVQSGAEVKKPGASVKVSCKASGYTFTRYDMHWVRQAPGQGLEWMGRIDPSTGGIKYAQK |
| | FQGRATLTVDTSTSTAYMELSRLRSDDTAVYYCARWDYGGYFDVWGQGTLVTVSS |
| 739 | EVQLVQSGAEVKKPGASVKVSCKASGYTFTSYWMHWVRQAPGQGLEWMGRIDPLSGDVKYAQK |
| | FQGRATLTVDTSTSTAYMELSRLRSDDTAVYYCARWDYGGYFDVWGHGTLVTVSS |
| 740 | EVQLVQSGAEVKKPGASVKVSCKASGYTFTSYLISWVRQAPGQGLEWMGRIDPQSGGVKYAQK |
| | FQGRATLTVDTSTSTAYMELSRLRSDDTAVYYCARWDYGGYFDVWGQGTLVTVSS |
| 741 | EVQLVQSGAEVKKPGASVKVSCKASGYTFTSYWLQWVRQAPGQGLEWMGRIDPESGGIKYAQK |
| | FQGRATLTVDTSTSTAYMELSRLRSDDTAVYYCARWDYGGYFDVWGQGTLVTVSS |
| 742 | EVQLVQSGAEVKKPGASVKVSCKASGYTFTSYLVNWVRQAPGQGLEWMGRIDPQSGGIKYAQK |
| | FQGRATLTVDTSTSTAYMELSRLRSDDTAVYYCARWDYGGYFDVWGQGTLVTVSS |
| 743 | EVOLVQSGAEVKKPGASVKVSCKASGYTFTNYLMQWVRQAPGQGLEWMGRIDPYRGGSIYAQK |
| | FQGRATLTVDTSTSTAYMELSRLRSDDTAVYYCARWDYGGYFDVWGQGTLVTVSS |
| 744 | EVQLVQSGAEVKKPGASVKVSCKASGYTFTRYFMTWVRQAPGQGLEWMGRIDPQGGGIKYAQK |
| | FQGRATLTVDTSTSTAYMELSRLRSDDTAVYYCARWDYGGYFDVWGQGTLVTVSS |
| 745 | EVOLVQSGAEVKKPGASVKVSCKASGYTFTSYLLTWVRQAPGQGLEWMGRIDPESGGRKYAQK |
| | FQGRATLTVDTSTSTAYMELSRLRSDDTAVYYCARWDYGGFFDVWGQGTLVTVSS |
| 746 | EVQLVQSGAEVKKPGASVKVSCKASGYTFTSYDMHWVRQAPGQGLEWMGRIDPQSGGFKYAQK |
| | FQGRATLTVDTSTSTAYMELSRLRSDDTAVYYCARWDYGGYFDVWGQGTLVTVSS |
| 747 | EVQLVQSGAEVKKPGASVKVSCKASGYTFTNYLLQWVRQAPGQGLEWMGRIDPESGGIKYAQK |
| | FQGRATLTVDTSTSTAYMELSRLRSDDTAVYYCARWDYGGYFDVWGQGTLVTVSS |
| 748 | EVQLVQSGAEVKKPGASVKVSCKASGYTFTRYYLNWVRQAPGQGLEWMGRIDPQSGGIKYAQK |
| | FQGRATLTVDTSTSTAYMELSRLRSDDTAVYYCARWDYGGYFDVWGQGTLVTVSS |
| 749 | EVQLVQSGAEVKKPGASVKVSCKASGYTFTSYLMHWVRQAPGQGLEWMGRIDPQNGGIKYAQK |
| | FQGRATLTVDTSTSTAYMELSRLRSDDTAVYYCARWDYGGYFDVWGQGTLVTVSS |
| 750 | EVQLGQSGAEVKKPGASVKVSCKASGYTFTSYLMTWVRQAPGQGLEWMGRIDPLSGGIKYAQK |
| | FQGRATLTVDTSTSTAYMELSRLRSDDTAVYYCARWDYGGYFDVWGQGTLVTVSS |
| 751 | EVQLVQSGAEVKKPGASVKVSCKASGYTFTRYLLQWVRQAPGQGLEWMGRIDPDSGAIKYAQK |
| | FQGRAALTVDTSTSTAYMELSRLRSDDTAVYYCARWDYGGYFDVWGQGTLVTVSS |
| 752 | EVQLVQSGAEVKKPGASVKVSCKASGYTFTSYLMQWVRQAPGQGLEWMGRIDPQSGGVKYAQK |
| | FQGRATLTVDTSTSTAYMELSRLRSDDTAVYYCARWDWGGYFDVWGQGTLVTVSS |
| 753 | EVQLVQSGAEVKKPGASVKVSCKASGYTFTSYLIQWVRQAPGQGLEWMGRIDPQSGGIKYAQK |
| | FQGRATLTVDTSTSTAYMELSRLRSDDTAVYYCARWDYGGYFDVWGQGTLVTVSS |
| 754 | EVQLVQSGAEVKKPGASVKVSCKASGYTFTSYWMQWVRQAPGQGLEWMGRIDPESGGVKYAQK |
| | FQGRATLTVDTSTSTAYMELSRLRSDDTAVYYCARWDWGGFFDVWGQGTLVTVSS |
| 755 | EVQLVQSGAEVKKPGASVKVSCKASGYTFTRYLMNWVRQAPGQGLEWMGRIDPQSGEGKYAQK |
| | FQGRATLTVDTSTSTAYMELSRLRSDDTAVYYCARWDYGGYFDVWGQGTLVTVSS |
| 756 | EVQLVQSGAEVKKPGASVKVSCKASGYTFTSYWMNWVRQAPGQGLEWMGRIDPQNGGIKYAQK |
| | FQGRATLTVDTSTSTAYMELSRLRSDDTAVYYCARWDYGGYFDVWGQGTLVTVSS |

TABLE 16
20E6 VL variants isolated from Doping Library after 2 hour off-rate selection,
P3 population

| Seq | |
|---|---|
| ID | |
| NO . | Amino acid sequence |
| 757 | DIQMTQSPSSLSASVGDRVTITCRASQDIYNYLNWYQQKPGKAVKLLIYYTSSLETGVPSRFS |
| | GSGSGTDYTLTISSLQPEDFATYFCQQGDTLPWTFGQGTKLEIK |
| 758 | DIQMTQSPSSLSASVGDRVTITCRASQDIYGYLNWYQQKPGKAVKLLIYYTSRLETGVPSRFS |
| | GSGSGTDYTLTISSLQPEDFATYFCQQGDTLPWTFGQGTKLEIK |
| 759 | DIQMTQSPSSLSASVGDRVTITCRASQDIYHYLNWYQQKPGKAVKLLIYYTSRLHSGVPSRFS |
| | GSGSGTDYTLTISSLQPEDFATYFCQQGDSLPWTFGQGTKLEIK |
| 760 | DIQMTQSPSSLSASVGDRVTITCRASQDIYNYLNWYQQKPGKAVKLLIYYTSSLETGVPSRFS |
| | GSGSGTDYTLTISSLQPEDFATYFCQQGDSMPWTFGQGTKLEIK |
| 761 | DIQMTQSPSSLSASVGDRVTITCRASQDIANYLNWYQQKPGKAVKLLIYYASGLETGVPSRFS |
| | GSGSGTDYTLTISSLQPEDFATYFCQQGDDLPWTFGQGTKLEIK |
| 762 | DIQMTQSPSSLSASVGDRVTITCRASQDIYNYLNWYQQKPGKAVKLLIYYTSRLETGVPSRFS |
| | GSGSGTDYTLTISSLQPEDFATYFCQQGDTLPWTFGQGTKLEIK |
| 763 | DIQMTQSPSSLSASVGDRVTITCRASQDIANYLNWYQQKPGKAVKLLIYYTSMLETGVPSRFS |
| | GSGSGTDYTLTISSLQPEDFATYFCQQGDTLPWTFGQGTKLEIK |
| 764 | DIQMTQSPSSLSASVGDRVTITCRASQDIYSYLNWYQQKPGKAVKLLIYYTSMLETGVPSRFS |
| | GSGSGTDYTLTISSLQPEDFATYFCQQGDTLPWTFGQGTKLEIK |
| 765 | DIQMTQSPSSLSASVGDRVTITCRASQDIFSYLNWYQQKPGKAVKLLIYYTSRLETGVPSRFS |
| | GSGSGTDYTLTISSLQPEDFATYFCQQGDTLPWTFGQGTKLEIK |
| 766 | DIQMTQSPSSLSASVGDRVTITCRASQDIFNYLNWYQQKPGKAVKLLIYYTSMLETGVPSRFS |
| | GSGSGTDYTLTISSLOPEDFATYFCQQGDAPPWTFGQGTKLEIK |
| 767 | DIQMTQSPSSLSASVGDRVTITCRASQDIYNYLNWYQQKPGKAVKLLIYYTSRLETGVPSRFS |
| | GSGSGTDYTLTISSLQPEDFATYFCQQGDALPWTFGQGTKLEIK |
| 768 | DIQMTQSPSSLSASVGDRVTITCRASQDITNYLNWYQQKPGKAVKLLIYYTSGLETGVPSRFS |
| | GSGSGTDYTLTISSLQPEDFATYFCQQGDILPWTFGQGTKLEIK |
| 769 | DIQMTQSPSSLSASVGDRVTITCRASQDITNYLNWYQQKPGKAVKLLIYYTSGLETGVPSRFS |
| | GSGSGTDYTLTISSLQPEDFATYFCQQGDALPWTFGQGTKLEIK |
| 770 | DIQMTQSPSSLSASVGDRVTITCRASQDIYNYLNWYQQKPGKAVKLLIYYTSRLETGVPSRES |
| | GSGSGTDYTLTISSLQPEDFATYFCQQGDSLPWTFGQGTKLEIK |
| 771 | DIQMTQSPSSLSASVGDRVTITCRASQDIFKYLNWYQQKPGKAVKLLIYYTSRLETGVPSRFS |
| | GSGSGTDYTLTISSLQPEDFATYFCQQGDTLPWTFGQGTKLEIK |
| 772 | DIQMTQSPSSLSASVGDRVTITCRASQDITNYLNWYQQKPGKAVKLLIYYSSRLETGVPSRFS |
| | GSGSGTDYTLTISSLQPEDFATYFCQQGDTLPWTFGQGTKLEIK |
| 773 | DIQMTQSPSSLSASVGDRVTITCRASQDIYNYLNWYQQKPGKAVKLLIYYSSRLETGVPSRFS |
| | GSGSGTDYTLTISSLQPEDFATYFCQQGDTLPWTFGQGTKLEIK |
| 774 | DIQMTQSPSSLSASVGDRVTITCRASQDITNYLNWYQQKPGKAVKLLIYYTSELETGVPSRFS |
| | GSGSGTDYTLTISSLOPEDFATYFCQQGDALPWTFGQGTKLEIK |
| 775 | DIQMTQSPSSLSASVGDRVTITCRASQDISYYLNWYQQKPGKAVKLLIYYSSLLETGVPSRFS |
| | GSGSGTDYTLTISSLQPEDFATYFCQQGDTLPWTFGQGTKLEIK |
| 776 | DIQMTQSPSSLSASVGDRVTITCRASQDIANYLNWYQQKPGKAVKLLIYYTSRLETGVPSRFS |
| | GSGSGTDYTLTISSLQPEDFATYFCQQGDTLPWTFGQGTKLEIK |
| 777 | DIQMTQSPSSLSASVGDRVTITCRASQDIFNYLNWYQQKPGKAVKLLIYYTSRLETGVPSRFS |
| | GSGSGTDYTLTISSLQPEDFATYFCQQGDILPWTFGQGTKLEIK |
| 778 | DIQMTQSPSSLSASVGDRVTITCRASQDITNYLNWYQQKPGKAVKLLIYYASMLETGVPSRFS |
| | GSGSGTDYTLTISSLOPEDFATYFCQQGDTVPWTFGQGTKLEIK |
| 779 | DIQMTQSPSSLSASVGDRVTITCRASQDIYNYLNWYQQKPGKAVKLLIYYSSRLHSGVPSRFS |
| | GSGSGTDYTLTISSLQPEDFATYFCQQGDTLPWTFGQGTKLEIK |
| 780 | DIQMTQSPSSLSASVGDRVTITCRASQDIYNYLNWYQQKPGKAVKLLIYYASRLETGVPSRFS |
| | GSGSGTDYTLTISSLQPEDFATYFCQQGDSLPWTFGQGTKLEIK |
| 781 | DIQMTQSPSSLSASVGDRVTITCRASQDIFNYLNWYQQKPGKAVKLLIYYTSRLHSGVPSRFS |
| | GSGSGTDYTLTISSLOPEDFATYFCQQGDTLPWTFGQGTKLEIK |
| 782 | DIQMTQSPSSLSASVGDRVTITCRASQDIYYYLNWYQQKPGKAVKLLIYYTSRLETGVPSRFS |
| | GSGSGTDYTLTISSLQPEDFATYFCQQGDTLPWTFGQGTKLEIK |
| 783 | DIQMTQSPSSLSASVGDRVTITCRASQDIFNYLNWYQQKPGKAVKLLIYYTSRLHSGVPSRFS |
| | GSGSGTDYTLTISSLQPEDFATYFCQQGDTVPWTFGQGTKLEIK |
| 784 | DIQMTQSPSSLSASVGDRVTITCRASQDITNYLNWYQQKPGKAVKLLIYYTSSLETGVPSRFS |
| | GSGSGTDYTLTISSLQPEDFATYFCQQGDTLPWTFGQGTKLEIK |
| 785 | DIQMTQSPSSLSASVGDRVTITCRASQDIFNYLNWYQQKPGKAVKLLIYYTSRLETGVPSRFS |
| | GSGSGTDYTLTISSLQPEDFATYFCQQGDYLPWTFGQGTKLEIK |
| 786 | DIQMTQSPSSLSASVGDRVTITCRASQDIANYLNWYQQKPGKAVKLLIYYTSELETGVPSRFS |
| | GSGSGTDYTLTISSLQPEDFATYFCQQGDSLPWTFGQGTKLEIK |
| 787 | DIQMTQSPSSLSASVGDRVTITCRASQDIFNYLNWYQQKPGKAVKLLIYYTSRLETGVPSRFS |
| | GSGSGTDYTLTISSLQPEDFATYFCQQGDTLPWTFGQGTKLEIK |
| 788 | DIQMTQSPSSLSASVGDRVTITCRASQDITNYLNWYQQKPGKAVKLLIYYTSRLHSGVPSRFS |
| | GSGSGTDYTLTISSLQPEDFATYFCQQGDTLPWTFGQGTKLEIK |
| 789 | DIQMTQSPSSLSASVGDRVTITCRASQDIYNYLNWYQQKPGKAVKLLIYYTSGLETGVPSRFS |
| | GSGSGTDYTLTISSLQPEDFATYFCQQGDTLPWTFGQGTKLEIK |
| 790 | DIQMTQSPSSLSASVGDRVTITCRASQDIYNYLNWYQQKPGKAVKLLIYYTSRLETGVPSRFS |
| | GSGSGTDYTLTISSLQPEDFATYFCQQGDGLPWTFGQGTKLEIK |
| 791 | DIQMTQSPSSLSASVGDRVTITCRASQDISNYLNWYQQKPGKAVKLLIYYTSGLETGVPSRFS |
| | GSGSGTDYTLTISSLQPEDFATYFCQQGDTLPWTFGQGTKLEIK |
| 792 | DIQMTQSPSSLSASVGDRVTITCRASQDIYNYLNWYQQKPGKAVKLLIYYTSRLETGVPSRFS |
| | GSGSGTDYTLTISSLQPEDFATYFCQQGDTVPWTFGQGTKLEIK |
| 793 | DIQMTQSPSSLSASVGDRVTITCRASQDIYNYLNWYQQKPGKAVKLLIYYTSNLETGVPSRFS |
| | GSGSGTDYTLTISSLQPEDFATYFCQQGDTLPWTFGQGTKLEIK |

TABLE 17
20E6 VH variants isolated from Doping Library 2 hour off-rate selection, P4
population

| Seq | |
|---|---|
| ID | Amino acid sequence |
| 794 | EVQLVQSGAEVKKPGASVKVSCKASGYTFTRYWMHWVRQAPGQGLEWMGRIDPPSGEIKYAQK |
| | FQGRATLTVDTSTSTAYMELSRLRSDDTAVYYCARWDYGGYFDVWGQGTLVTVSS |
| 795 | EVQLVQSGAEVKKPGASVKVSCKASGYTFTRYLMQWVRQAPGQGLEWMGRIDPDSGGSRYAQK |
| | FQGRATLTVDTSTSTAYMELSRLRSDDTAVYYCARWDYGGYFDVWGQGTLVTVSS |
| 796 | EVQLVQSGAEVKKPGASVKVSCKASGYTFTRYWMSWVRQAPGQGLEWMGRIDPQSGSFKYAQK |
| | FQGRATLTVDTSTSTAYMELSRLRSDDTAVYYCARWDYGGYFDVWGQGTLVTVSS |
| 797 | EVQLVQSGAEVKKPGASVKVSCKASGYTFTSYLMQWVRQAPGQGLEWMGRIDPQSGGIRYAQK |
| | FQGRATLTVDTSTSTAYMELSRLRSDDTAVYYCARWDYGGYFDVWGQGTLVTVSS |
| 798 | EVQLVQSGAEVKKPGASVKVSCKASGYTFTTYLMQWVRQAPGQGLEWMGRIDPQSGGFIYAQK |
| | FQGRATLTVDTSTSTAYMELSRLRSDDTAVYYCARWDYGGYFDVWGQGTLVTVSS |
| 799 | EVQLVQSGAEVKKPGASVKVSCKASGYTFTSYLVQWVRQAPGQGLEWMGRIDPESGGSRYAQK |
| | FQGRATLTVDTSTSTAYMELSRLRSDDTAVYYCARWDYGGYFDVWGQGTLVTVSS |
| 800 | EVQLVQSGAEVKKPGASVKVSCKASGYTFTNYYMNWVRQAPGQGLEWMGRIDPQSGGIIYAQK |
| | FQGRATLTVDTSTSTAYMELSRLRSDDTAVYYCARWNHGGYFDVWGQGTLVTVSS |
| 801 | EVQLVQSGAEVKKPGASVKVSCKASGYTFTRYWMQWVRQAPGQGLEWMGRIDPQNGVSKYAQK |
| | FQGRATLTVDTSTSTAYMELSRLRSDDTAVYYCARWDYGGYFDVWGQGTLVTVSS |
| 802 | EVQLVQSGAEVKKPGASVKVSCKASGYTFTSYLMNWVRQAPGQGLEWMGRIDPQSGGTKYAQK |
| | FQGRATLTVDTSTSTAYMELSRLRSDDTAVYYCARWDYGGYFDVWGQGTLVTVSS |
| 803 | EVQLVQSGAEVKKPGASVKVSCKASGYTFTSYDMHWVRQAPGQGLEWMGRIDLQGGVNRYAQK |
| | FQGRATLTVDTSTSTAYMELSRLRSDDTAVYYCARWDYGGYFDVWGQGTLVTVSS |
| 804 | EVOLVQSGAEVKKPGASVKVSCKASGYTFTTYSMQWVRQAPGQGLEWMGRIDPQSGGIKYAQK |
| | FQGRATLTVDTSTSTAYMELSRLRSDDTAVYYCARWDYGGYFDVWGQGTLVTVSS |
| 805 | EVOLVQSGAEVKKPGASVKVSCKASGYTFTSYYMNWVRQAPGQGLEWMGRIDPQEGGGKYAQK |
| | FQGRATLTVDTSTSTAYMELSRLRSDDTAVYYCARWDYGGYFDVWGQGTLVTVSS |
| 806 | EVQLVQSGAEVKKPGASVKVSCKASGYTFTSYLVQWVRQAPGQGLEWMGRIDPVSGGITYAQK |
| | FQGRATLTVDTSTSTAYMELSRLRSDDTAVYYCARWDYGGYFDVWGQGTLVTVSS |
| 807 | EVQLVQSGAEVKKPGASVKVSCKASGYTFTSYLMHWVRQAPGQGLEWMGRIDPESGGIKYAQK |
| | FQGRATLTVDTSTSTAYMELSRLRSDDTAVYYCARWDYGGYFDVWGQGTLVTVSS |
| 808 | EVQLVQSGAEVKKPGASVKVSCKASGYTFTRYLMHWVRQAPGQGLEWMGRIDPQSGGIKYAQK |
| | FQGRATLTVDTSTSTAYMELSRLRSDDTAVYYCARWDYGGYFDVWGQGTLVTVSS |
| 809 | EVQLVQSGAEVKKPGASVKVSCKASGYTFTSYLMQWVRQAPGQGLEWMGRIDPSSGGIGYAQK |
| | FQGRATLTVDTSTSTAYMELSRLRSDDTAVYYCARWDFGGYFDVWGQGTLVTVSS |
| 810 | EVQLVQSGAEVKKPGASVKVSCKASGYTFTSYWMQWVRQAPGQGLEWMGRIDPVSGGVKYAQK |
| | FQGRATMTVDTSTSTAYMELSRLRSDDTAVYYCARWDYGGYFDVWGQGTLVTVSS |
| 811 | EVOLVQSGAEVKKPGASVKVSCKASGYTFTRYLTHWVRQAPGQGLEWMGRIDPQIGGITYAQK |
| | FQGRATLTVDTSTSTAYMELSRLRSDDTAVYYCARWDYGGYFDVWGQGTLVTVSS |
| 812 | EVQLVQSGAEVKKPGASVKVSCKASGYTFTRYWMHWVRQAPGQGLEWMGRIDPTSGGIRYAQK |
| | FQGRATLTVDTSTSTAYMELSRLRSDDTAVYYCARWDYGGFFDVWGQGTLVTVSS |
| 813 | EVQLVQSGAEVKKPGASVKVSCKASGYTFTSYLINWVRQAPGQGLEWMGRIDPQSGGVKYAQK |
| | FQGRATLTVDTSTSTAYMELSRLRSDDTAVYYCARWDYGGYFDVWGQGTLVTVSS |
| 814 | EVQLVQSGAEVKKPGASVKVSCKASGYTFTSYWMQWVRQAPGQGLEWMGRIDPQSGGIKYAQK |
| | FQGRATLTVDTSTSTAYMELSRLRSDDTAVYYCARWDWGGYFDVWGQGTLVTVSS |
| 815 | EVQLVQSGAEVKKPGASVKVSCKASGYTFTRYLMHWVRQAPGQGLEWMGRIDPESGGVIYAQK |
| | FQGRATLTVDTSTSTAYMELSRLRSDDTAVYYCARWDYGGYFDVWGQGTLVTVSS |
| 816 | EVQLVQSGAEVKKPGASVKVSCKASGYTFTGYYMNWVRQAPGQGLEWMGRIDPRSGGFRYAQK |
| | FQGRATLTVDTSTSTAYMELSRLRSDDTAVYYCARWDYGGYFDVWGQGTLVTVSS |
| 817 | EVQLVQSGAEVKKPGASVKVSCKASGYTFTSYLLQWVRQAPGQGLEWMGRIDPMTGGMMYAQK |
| | FQGRATLTVDTSTSTAYMELSRLRSDDTAVYYCARWDYGGYFDVWGQGTLVTVSS |
| 818 | EVQLVQSGAEVKKPGASVKVSCKASGYTFTSYWMHWVRQAPGQGLEWMGRIDPHSGGKKYAQK |
| | FQGRATLTVDTSTSTAYMELSRLRSDDTAVYYCARWDYGGYFDVWGQGTLVTVSS |
| 819 | EVQLVQSGAEVKKPGASVKVSCKASGYTFTRYWMHWVRQAPGQGLEWMGRIDPQSGGMKYAQK |
| | FQGRATLTVDTSTSTAYMELSRLRSDDTAVYYCARWDYGGYFDVWGQGTLVTVSS |
| 820 | EVQLVQSGAEVKKPGASVKVSCKASGYTFTSYWLNWVRQAPGQGLEWMGRIDPQSGGVRYAQK |
| | FQGRATLTVDTSTSTAYMELSRLRSDDTAVYYCARWDYGGYFDVWGQGTLVTVSS |
| 821 | EVQLVQSGAEVKKPGASVKVSCKASGYTFTRYGMNWVRQAPGQGLEWMGRIDPQSGGNKYAQK |
| | FQGRATLTVDTSTSTAYMELSRLRSDDTAVYYCARWDWGGYFDVWGQGTLVTVSS |
| 822 | EVQLVQSGAEVKKPGASVKVSCKASGYTFTSYLMHWVRQAPGQGLEWMGRIDPHRGDSYYAQK |
| | FQGRATLTVDTSTSTAYMELSRLRSDDTAVYYCARWDWGGYFDVWGQGTLVTVSS |
| 823 | EVQLVQSGAEVKKPGASVKVSCKASGYTFTRYLLAWVROAPGQGLEWMGRIDPQSGGIRYAQK |
| | FQGRATLTVDTSTSTAYMELSRLRSDDTAVYYCARWDYGGYFDVWGQGTLVTVSS |
| 824 | EVQLVQSGAEVKKPGASVKVSCKASGYTFTSYWMHWVRQAPGQGLEWMGRIDPLSGGILYAQK |
| | FQGRATLTVDTSTSTAYMELSRLRSDDTAVYYCARWDYGGYFDVWGQGTLVTVSS |
| 825 | EVQLVQSGAEVKKPGASVKVSCKASGYTFTSYLIHWVRQAPGQGLEWMGRIDPESGGIKYAQK |
| | FQGRATLTVDTSTSTAYMELSRLRSDDTAVYYCARWDYGGYFDVWGQGTLVTVSS |
| 826 | EVQLVQSGAEVKKPGASVKVSCKASGYTFTSYLLQWVRQAPGQGLEWMGRIDPRNGGIIYAQK |
| | FQGRATLTVDTSTSTAYMELSRLRSDDTAVYYCARWDWGGYFDVWGQGTLVTVSS |
| 827 | EVQLVQSGAEVKKPGASVKVSCKASGYTFTRYWTHWVRQAPGQGLEWMGRIDPLHGGSKYAQK |
| | FQGRATLTVDTSTSTAYMELSRLRSDDTAVYYCARWDHGGYFDVWGQGTLVTVSS |

TABLE 18
20E6 VL variants isolated from Doping Library after 2 hour off-rate selection,
P4 population

| Seq | |
|---|---|
| ID | |
| NO. | Amino acid sequence |
| 828 | DIQMTQSPSSLSASVGDRVTITCRASQDITNYLNWYQQKPGKAVKLLIYYTSGLETGVPSRFS |
| | GSGSGTDYTLTISSLQPEDFATYFCQQGDTLPWTFGQGTKLEIK |
| 829 | DIQMTQSPSSLSASVGDRVTITCRASQDIYHYLNWYQQKPGKAVKLLIYYSSMLETGVPSRFS |
| | GSGSGTDYTLTISSLOPEDFATYFCQQGDTTPWTFGQGTKLEIK |
| 830 | DIQMTQSPSSLSASVGDRVTITCRASQDIYNYLNWYQQKPGKAVKLLIYYNSKLETGVPSRFS |
| | GSGSGTDYTLTISSLQPEDFATYFCQQGDTLPWTFGQGTKLEIK |
| 831 | DIQMTQSPSSLSASVGDRVTITCRASQDIYNYLNWYQQKPGKAVKLLIYYTSSLETGVPSRFS |
| | GSGSGTDYTLTISSLQPEDFATYFCQQGDSLPWTFGQGTKLEIK |
| 832 | DIQMTQSPSSLSASVGDRVTITCRASQDIYNYLNWYQQKPGKAVKLLIYYTSQLETGVPSRFS |
| | GSGSGTDYTLTISSLQPEDFATYFCQQGDTPPWTFGQGTKLEIK |
| 833 | DIQMTQSPSSLSASVGDRVTITCRASQDIYHYLNWYQQKPGKAVKLLIYYTSRLHSGVPSRFS |
| | GSGSGTDYTLTISSLQPEDFATYFCQQGDILPWTFGQGTKLEIK |
| 834 | DIQMTQSPSSLSASVGDRVTITCRASQDITNYLNWYQQKPGKAVKLLIYYTSMLETGVPSRFS |
| | GSGSGTDYTLTISSLQPEDFATYFCQQGDTLPWTFGQGTKLEIK |
| 835 | DIQMTQSPSSLSASVGDRVTITCRASQDINKYLNWYQQKPGKAVKLLIYYTSRLETGVPSRFS |
| | GSGSGTDYTLTISSLQPEDFATYFCQQGDILPWTFGQGTKLEIK |
| 836 | DIQMTQSPSSLSASVGDRVTITCRASQDIANYLNWYQQKPGKAVKLLIYYTSRLHSGVPSRFS |
| | GSGSGTDYTLTISSLQPEDFATYFCQQGDTLPWTFGQGTKLEIK |
| 837 | DIQMTQSPSSLSASVGDRVTITCRASQDIFNYLNWYQQKPGKAVKLLIYYSSMLETGVPSRFS |
| | GSGSGTDYTLTISSLQPEDFATYFCQQGDAQPWTFGQGTKLEIK |
| 838 | DIQMTQSPSSLSASVGDRVTITCRASQDITNYLNWYQQKPGKAVKLLIYYTSILETGVPSRFS |
| | GSGSGTDYTLTISSLQPEDFATYFCQQGDTLPWTFGQGTKLEIK |
| 839 | DIQMTQSPSSLSASVGDRVTITCRASQDIYNYLNWYQQKPGKAVKLLIYYTSSLETGVPSRFS |
| | GSGSGTDYTLTISSLQPEDFATYFCQQGDTLPWTFGQGTKLEIK |
| 840 | DIQMTQSPSSLSASVGDRVTITCRASQDIYNYLNWYQQKPGKAVKLLIYYTSSLHSGVPSRFS |
| | GSGSGTDYTLTISSLQPEDFATYFCQQGDTLPWTFGQGTKLEIK |
| 841 | DIQMTQSPSSLSASVGDRVTITCRASQDITNYLNWYQQKPGKAVKLLIYYTSRLETGVPSRFS |
| | GSGSGTDYTLTISSLQPEDFATYFCQQGDTLPWTFGQGTKLEIK |
| 842 | DIQMTQSPSSLSASVGDRVTITCRASQDIYKYLNWYQQKPGKAVKLLIYYTSRLETGVPSRFS |
| | GSGSGTDYTLTISSLQPEDFATYFCQQGDTLPWTFGQGTKLEIK |
| 843 | DIQMTQSPSSLSASVGDRVTITCRASQDITNYLNWYQQKPGKAVKLLIYYTSLLETGVPSRFS |
| | GSGSGTDYTLTISSLQPEDFATYFCQQGDTVPWTFGQGTKLEIK |
| 844 | DIQMTQSPSSLSASVGDRVTITCRASQDIYNYLNWYQQKPGKAVKLLIYYTSRLHSGVPSRFS |
| | GSGSGTDYTLTISSLQPEDFATYFCQQGDTLPWTFGQGTKLEIK |
| 845 | DIQMTQSPSSLSASVGDRVTITCRASQDIYNYLNWYQQKPGKAVKLLIYYSSRLETGVPSRFS |
| | GSGSGTDYTLTISSLQPEDFATYFCQQGDTLPWTFGQGTKLEIK |
| 846 | DIQMTQSPSSLSASVGDRVTITCRASQDITYYLNWYQQKPGKAVKLLIYYTSWLETGVPSRFS |
| | GSGSGTDYTLTISSLQPEDFATYFCQQGDALPWTFGQGTKLEIK |
| 847 | DIQMTQSPSSLSASVGDRVTITCRASQDIANYLNWYQQKPGKAVKLLIYYTSGLETGVPSRFS |
| | GSGSGTDYTLTISSLQPEDFATYFCQQGDILPWTFGQGTKLEIK |
| 848 | DIQMTQSPSSLSASVGDRVTITCRASQDITNYLNWYQQKPGKAVKLLIYYKSRLETGVPSRFS |
| | GSGSGTDYTLTISSLOPEDFATYFCQQGDILPWTFGQGTKLEIK |

TABLE 19
20E6 VH variants isolated from Doping Library
after 12 hour off-rate selection

| Seq | |
|---|---|
| ID | |
| NO. | Amino acid sequence |
| 849 | EVQLVQSGAEVKKPGASVKVSCKASGYTFTRYLVQWVRQAPGQGLEWMGRIDPQCGDLKYAQK |
| | FQGRATLTVDTSTSTAYMELSRLRSDDTAVYYCARWDYGGYFDVWGQGTLVTVSS |
| 850 | EVQLVQSGAEVKKPGASVKVSCKASGYTFTRYYLNWVRQAPGQGLEWMGRIDPQSGGTKYAQK |
| | FQGRATLTVDTSTSTAYMELSRLRSDDTAVYYCARWDWGGYFDVWGQGTLVTVSS |
| 851 | EVQLVQSGAEVKKPGASVKVSCKASGYTFTRYLNOWVRQAPGQGLEWMGRIDPQNGEINYAQK |
| | FQGRATLTVDTSTSTAYMELSRLRSDDTAVYYCARWDYGGYFDVWGQGTLVTVSS |
| 852 | EVQLVQSGAEVKKPGASVKVSCKASGYTFTSYLQSWVRQAPGQGLEWMGRIDPQSGGSRYAQK |
| | FQGRATLTVDTSTSTAYMELSRLRSDDTAVYYCARWDWGGQFDVWGQGTLVTVSS |
| 853 | EVQLVQSGAEVKKPGASVKVSCKASGYTFTSYYMNWVRQAPGQGLEWMGRIDPESGGSKYAQK |
| | FQGRATLTVDTSTSTAYMELSRLRSDDTAVYYCARWDYGGYFDVWGQGTLVTVSS |
| 854 | EVQLVQSGAEVKKPGASVKVSCKASGYTFTTYYMNWVRQAPGQGLEWMGRIDPLNGGVRYAQK |
| | FQGRATLTVDTSTSTAYMELSRLRSDDTAVYYCARWDYGGYFDVWGQGTLVTVSS |
| 855 | EVQLVQSGAEVKKPGASVKVSCKASGYTFTSYLVQWVRQAPGQGLEWMGRIDPQDGGVRYAQK |
| | FQGRATLTVDTSTSTAYMELSRLRSDDTAVYYCARWDYGGYFDVWGQGTLVTVSS |
| 856 | EVQLVQSGAEVKKPGASVKVSCKASGYTFTSYYMQWVRQAPGQGLEWMGRIDPESGGVKYAQK |
| | FQGRATLTVDTSTSTAYMELSRLRSDDTAVYYCARWDYGGYFDVWGQGTLVTVSS |
| 857 | EVQLVQSGAEVKKPGASVKVSCKASGYTFTNYYINWVRQAPGQGLEWMGRIDPQSGGVKYAQK |
| | FQGRATLTVDTSTSTAYMELSRLRSDDTAVYYCARWDYGGYFDVWGQGTLVTVSS |
| 858 | EVQLVQSGAEVKKPGASVKVSCKASGYTFTIYDMHWVRQAPGQGLEWMGRIDPQSGGIKYAQK |
| | FQGRATLTVDTSTSTAYMELSRLRSDDTAVYYCARWDYGGYFDVWGQGTLVTVSS |
| 859 | EVQLVQSGAEVKKPGASVKVSCKASGYTFTRYYLSWVRQAPGQGLEWMGRIDPHIGGIKYAQK |
| | FQGRATLTVDTSTSTAYMELSRLRSDDTAVYYCARWDWGGYFDVWGQGTLVTVSS |
| 860 | EVQLVQSGAEVKKPGASVKVSCKASGYTFTSYFMNWVRQAPGQGLEWMGRIDPLDGSIKYAQK |
| | FQGRATLTVDTSTSTAYMELSRLRSDDTAVYYCARWDYGGYFDVWGQGTLVTVSS |
| 861 | EVQLVQSGAEVKKPGASVKVSCKASGYTFTSYLMQWVRQAPGQGLEWMGRIDPQIGDSIYAQK |
| | FQGRATLTVDTSTSTAYMELSRLRSDDTAVYYCARWDWGGYFDVWGQGTLVTVSS |
| 862 | EVQLVQSGAEVKKPGASVKVSCKASGYTFTSYYMNWVRQAPGQGLEWMGRIDPQSGGVKYAQK |
| | FQGRATLTVDTSTSTAYMELSRLRSDDTAVYYCARWDYGGYFDVWGQGTLVTVSS |
| 863 | EVQLVQSGAEVKKPGASVKVSCKASGYTFTSYYINWVRQAPGQGLEWMGRIDPQSGDIKYAQK |
| | FQGRATLTVDTSTSTAYMELSRLRSDDTAVYYCARWDYGGYFDVWGQGTLVTVSS |
| 864 | EVQLVQSGAEVKKPGASVKVSCKASGYTFTGYFLNWVRQAPGQGLEWMGRIDPQSGGRIYAQK |
| | FQGRATLTVDTSTSTAYMELSRLRSDDTAVYYCARWDWGGYFDVWGQGTLVTVSS |
| 865 | EVQLVQSGAEVKKPGASVKVSCKASGYTFTRYYTNWVRQAPGQGLEWMGRIDPQTGGIRYAQK |
| | FQGRATLTVDTSTSTAYMELSRLRSDDTAVYYCARWDYGGYFDVWGQGTLVTVSS |
| 866 | EVQLVQSGAEVKKPGASVKVSCKASGYTFTSYWMQWVRQAPGQGLEWMGRIDPQIGDTKYAQK |
| | FQGRATLTVDTSTSTAYMELSRLRSDDTAVYYCARWDYGGYFDVWGQGTLVTISS |
| 867 | EVQLVQSGAEVKKPGASVKVSCKASGYTFTRYWMHWVRQAPGQGLEWMGRIDPQDGGIIYAQK |
| | FQGRATLTVDTSTSTAYMELSRLRSDDTAVYYCARWDYGGYFDVWGQGTLVTVSS |
| 868 | EVRLVQSGAEVKKPGASVKVSCKASGYTFTRYWLQWVRQASGQGLEWMGRIDPLDGGTKYAQK |
| | FQGRATLTVDTSTSTAYMELSRLRSDDTAVYYCARWDYGGYFDVWGQGTLVTVSS |
| 869 | EVQLVQSGAEVKKPGASVKVSCKASGYTFTYYDVHWVRQAPGQGLEWMGRIDPHDGGIKYAQK |
| | FQGRATLTVDTSTSTAYMELSRLRSDDTAVYYCARWDYGGYFDVWGQGTLVTVSS |
| 870 | EVQLVQSGAEVKKPGASVKVSCKASGYTFTGYYVNWVRQAPGQGLEWMGRIDPESGGMLYAQK |
| | FQGRATLTVDTSTSTAYMELSRLRSDDTAVYYCARWDYGGYFDVWGQGTLVTVSS |
| 871 | EVQLVQSGAEVKKPGASVKVSCKASGYTFTRYWLQWVRQAPGQGLEWMGRIDPQGGDIKYAQK |
| | FQGRATLTVDTSTSTAYMELSRLRSDDTAVYYCARWDYGGYFDVWGHGTLVTVSS |
| 872 | EVQLVQSGAEVKKPGASVKVSCKASGYTFTSYDMHWVRQAPGQGLEWMGRIDPQSGGTRYAQK |
| | FQGRATLTVDTSTSTAYMELSRLRSDDTAVYYCARWDYGGYFDVWGQGTLVTVSS |
| 873 | EVQLVQSGAEVKKPGASVKVSCKASGYTFTSYYMNWVRQAPGQGLEWMGRIDPQSGGNKYAQK |
| | FQGRATLTVDTSTSTAYMELSRLRSDDTAVYYCARWDYGGYFDVWGQGTLVTVSS |
| 874 | EVQLVQSGAEVKKPGASVKVSCKASGYTFTSYDIHWVRQAPGQGLEWMGRIDPESGGIKYAQK |
| | FQGRATLTVDTSTSTAYMELSRLRSDDTAVYYCARWDYGGYFDVWGQGTLVTVSS |
| 875 | EVQLVQSGAEVKKPGASVKVSCKASGYTFTSYLIQWVRQAPGQGLEWMGRIDPQSGGVKYAQK |
| | FQGRATLTVDTSTSTAYMELSRLRSDDTAVYYCARWDYGGQFDVWGQGTLVSVSS |
| 876 | EVQLVQSGAEVKKPGASVKVSCKASGYTFTSYYLNWVRQAPGQGLEWMGRIDPQGGGLKYAQK |
| | FQGRATLTVDTSTSTAYMELSRLRSDDTAVYYCARWDYGGYFDVWGQGTLVTVSS |
| 877 | EVQLVQSGAEVKKPGASVKVSCKASGYTFTSYYLNWVRQAPGQGLEWMGRIDPKSGDIIYAQK |
| | FQGRATLTVDTSTSTAYMELSRLRSDDTAVYYCARWDYGGYFDVWGQGTLVTVSS |
| 878 | EVQLVQSGAEVKKPGASVKVSCKASGYTFTRYYVNWVRQAPGQGLEWMGRIDPESGGIKYAQK |
| | FQGRATLTVDTSTSTAYMELSRLRSDDTAVYYCARWDYGGYFDVWGQGTLVTVSS |
| 879 | EVOLVQSGAEVKKPGASVKVSCKASGYTFTTYYMSWVRQAPGQGLEWMGRIDPQSGGSKYAQK |
| | FQGRATLTVDTSTSTAYMELSRLRSDDTAVYYCARWDYGGYFDVWGQGTLVTVSS |
| 880 | EVQLVQSGAEVKKPGASVKVSCKASGYTETTYFMNWVRQAPGQGLEWMGRIDPQSGGIKYAQK |
| | FQGRATLTVDTSTSTAYMELSRLRSDDTAVYYCARWDYGGYFDVWGQGTLVTVSS |
| 881 | EVQLVQSGAEVKKPGASVKVSCKASGYTFTRYLMQWVRQAPGQGLEWMGRIDPQDGGIKYAQK |
| | FQGRATLTVDTSTSTAYMELSRLRSDDTAVYYCARWDYGGYFDVWGQGTLVTVSS |
| 882 | EVQLVZSGAEVKKPGASVKVSCKZSGYTFTSYLLSWVRQAPGQGLEWMGRIDPQSGGIRYAQK |
| | FQGRATLTVDTSTSTAYMELSRLRSDDTAVYYCARWDYGGYFDVWGQGTLVTVSS |
| 883 | EVQLVQSGAEVKKPGASVKVSCKASGYTFTRYWMQWVRQAPGQGLEWMGRIDPQSGDVRYAQK |
| | FQGRATLTVDTSTSTAYMELSRLRSDDTAVYYCARWDYGGYFDVWGQGTLVTVSS |
| 884 | EVQLVQSGAEVKKPGASVKVSCKASGYTFTTYYMHWVRQAPGQGLEWMGRIDPQIGESKYAQK |
| | FQGRATLTVDTSTSTAYMELSRLRSDDTAVYYCARWDYGGYFDVWGQGTLVTVSS |
| 885 | EVQLVQSGAEVKKPGASVKVSCKASGYTFTSYLINWVRQAPGQGLEWMGRIDPEDGGNKYAQK |
| | FQGRATLTVDTSTSTAYMELSRLRSDDTAVYYCARWDYGGYFDVWGHGTLVTVSS |
| 886 | EVQLVQSGAEVKKPGASVKVSCKASGYTFTSYYMTWVRQAPGQGLEWMGRIDPQSGGGKYAQK |
| | FQGRATLTVDTSTSTAYMELSRLRSDDTAVYYCARWDYGGQFDVWGQGTLVTVSS |
| 887 | EVQLVQSGAEVKKPGASVKVSCKASGYTETRYWIQWVRQAPGQGLEWMGRIDPQIGEKRYAQK |
| | FQGRATLTVDTSTSTAYMELSRLRSDDTAVYYCARWDYGGYFDVWGQGTLVTVSS |
| 888 | EVQLVQSGAEVKKPGASVKVSCKASGYTFTSYDTHWVRQAPGQGLEWMGRIDPENGGTKYAQK |
| | FQGRATLTVDTSTSTAYMELSRLRSDDTAVYYCARWDWGGYFDVWGQGTLVTVSS |
| 889 | EVQLVQSGAEVKKPGASVKVSCKASGYTETTYDMHWVRQAPGQGLEWMGRIDPQSGGIKYAQK |
| | FQGRATLTVDTSTSTAYMELSRLRSDDTAVYYCARWDYGGYFDVWGQGTLVTVSS |
| 890 | EVQLVQSGAEVKKPGASVKVSCKASGYTFTSYYMNWVRQAPGQGLEWMGRIDPESGGIKYAQK |
| | FQGRATLTVDTSTSTAYMELSRLRSDDTAVYYCARWDYGGYFDVWGQGTLVTVSS |
| 891 | EVQLVQSGAEVKKPGASVKVSCKASGYTFTSYYMNWVRQAPGQGLEWMGRIDPQSGGIIYAQK |
| | FQGRATLTVDTSTSTAYMELSRLRSDDTAVYYCARFDYGGYFDVWGQGTLVTVSS |
| 892 | EVQLVQSGAEVKKPGASVKVSCKASGYTFTRYDLHWVRQAPGQGLEWMGRIDPISGGIKYAQK |
| | FQGRATLTVDTSTSTAYMELSRLRSDDTAVYYCARWDYGGYFDVWGQGTLVTVSS |
| 893 | EVQLVQSGAEVKKPGASVKVSCKASGYTFTRYWIQWVRQAPGQGLEWMGRIDPQDGGIKYAQK |
| | FQGRATLTVDTSTSTAYMELSRLRSDDTAVYYCARWDYGGYFDVWGQGTLVTVSS |

TABLE 20
20E6 VL variants isolated from Doping Library after
12 hour off-rate selection

| Seq | |
|---|---|
| ID | |
| NO. | Amino acid sequence |
| 894 | DIQMTQSPSSLSASVGDRVTITCRASQDIWKYLNWYQQKPGKAVKLLIYYTSILETGVPSRFS |
| | GSGSGTDYTLTISSLQPEDFATYFCQQGDSLPWTFGQGTKLEIK |
| 895 | DIQMTQSPSSLSASVGDRVTITCRASQDIWNYLNWYQQKPGKAVKLLIYYTSILETGVPSRFS |
| | GSGSGTDYTLTISSLQPEDFATYFCQQGDTLPWTFGQGTKLDIK |
| 896 | DIQMTQSPSSLSASVGDRVTITCRASQDIWNYLNWYQQKPGKAVKLLIYYTSILHSGVPSRFS |
| | GSGSGTDYTLTISSLQPEDFATYFCQQGDTLPWTFGQGTKLEIK |
| 897 | DIQMTQSPSSLSASVGDRVTITCRASQDIWNYLNWYQQKPGKAVKLLIYYTSMLETGVPSRFS |
| | GSGSGTDYTLTISSLQPEDFATYFCQQGDSLPWTFGQGTKLEIK |
| 898 | DIQMTQSPSSLSASVGDRVTITCRASQDIWHYLNWYQQKPGKAVKLLIYYTSELETGVPSRFS |
| | GSGSGTDYTLTISSLOPEDFATYFCQQGDTLPWTFGQGTKLEIK |
| 899 | DIQMTQSPSSLSASVGDRVTITCRASQDIWNYLNWYQQKPGKAVKLLIYYTSRLETGVPSRFS |
| | GSGSGTDYTLTISSLOPEDFATYFCQQGDTLPWTFGQGTKLEIK |
| 900 | DIQMTQSPSSLSASVGDRVTITCRASQDIWNYLNWYQQKPGKAVKLLIYYTSGLHSGVPSRFS |
| | GSGSGTDYTLTISSLQPEDFATYFCQQGDILPWTFGQGTKLEIK |
| 901 | DIQMTQSPSSLSASVGDRVTITCRASQDIWKYLNWYQQKPGKAVKLLIYYTSGLETGVPSRFS |
| | GSGSGTDYTLTISSLQPEDFATYFCQQGDTLPWTFGQGTKLEIK |
| 902 | DIQMTQSPSSLSASVGDRVTITCRASQDIWNYLNWYQQKPGKAVKLLIYYTSMLHSGVPSRFS |
| | GSGSGTDYTLTISSLQPEDFATYFCQQGDSLPWTFGQGTKLEIK |
| 903 | DIQMTQSPSSLSASVGDRVTITCRASQDIWNYLNWYQQKPGKAVKLLIYYTSGLETGVPSRFS |
| | GSGSGTDYTLTISSLQPEDFATYFCQQGDSLPWTFGQGTKLEIK |
| 904 | DIQMTQSPSSLSASVGDRVTITCRASQDIWNYLNWYQQKPGKAVKLLIYYTSMLHSGVPSRFS |
| | GSGSGTDYTLTISSLQPEDFATYFCQQGDTLPWTFGQGTKLEIK |
| 905 | DIQMTQSPSSLSASVGDRVTITCRASQDIWNYLNWYQQKPGKAVKLLIYYTSGLETGVPSRFS |
| | GSGSGTDYTLTISSLQPEDFATYFCQQGDTLPWTFGQGTKLEIK |
| 906 | DIQMTQSPSSLSASVGDRVTITCRASQDIWNYLNWYQQKPGKAVKLLIYYTSRLETGVPSRFS |
| | GSGSGTDYTLTISSLQPEDFATYFCQQGDNLPWTFGQGTKLEIK |
| 907 | DIQMTQSPSSLSASVGDRVTITCRASQDIWNYLNWYQQKPGKAVKLLIYYTSRLHSGVPSRFS |
| | GSGSGTDYTLTISSLQPEDFATYFCQQGDALPWTFGQGTKLEIK |
| 908 | DIQMTQSPSSLSASVGDRVTITCRASQDIWNYLNWYQQKPGKAVKLLIYYTSVLETGVPSRFS |
| | GSGSGTDYTLTISSLOPEDFATYFCQQGDNLPWTFGQGTKLEIK |
| 909 | DIQMTQSPSSLSASVGDRVTITCRASQDIWNYLNWYQQKPGKAVKLLIYYTSVLETGVPSRFS |
| | GSGSGTDYTLTISSLQPEDFATYFCQQGDELPWTFGQGTKLEIK |
| 910 | DIQMTQSPSSLSASVGDRVTITCRASQDIWNYLNWYQQKPGKAVKLLIYYTSMLETGVPSRFS |
| | GSGSGTDYTLTISSLQPEDFATYFCQQGDTLPWTFGQGTKLEIK |
| 911 | DIQMTQSPSSLSASVGDRVTITCRASQDIWNYLNWYQQKPGKAVKLLIYYTSMLHSGVPSRFS |
| | GSGSGTDYTLTISSLQPEDFATYFCQQGDMLPWTFGQGTKLEIK |
| 912 | DIQMTQSPSSLSASVGDRVTITCRASQDIWNYLNWYQQKPGKAVKLLIYYTSLLETGVPSRFS |
| | GSGSGTDYTLTISSLOPEDFATYFCQQGDTLPWTFGQGTKLEIK |
| 913 | DIQMTQSPSSLSASVGDRVTITCRASQDIWNYLNWYQQKPGKAVKLLIYYTSKLETGVPSRFS |
| | GSGSGTDYTLTISSLQPEDFATYFCQQGDSLPWTFGQGTKLEIK |
| 914 | DIQMTQSPSSLSASVGDRVTITCRASQDIWHYLNWYQQKPGKAVKLLIYYTSMLETGVPSRFS |
| | GSGSGTDYTLTISSLQPEDFATYFCQQGDTLPWTFGQGTKLEIK |
| 915 | DIQMTQSPSSLSASVGDRVTITCRASQDIWHYLNWYQQKPGKAVKLLIYYTSMLETGVPSRFS |
| | GSGSGTDYTLTISSLQPEDFATYFCQQGDILPWTFGQGTKLEIK |
| 916 | DIQMTQSPSSLSASVGDRVTITCRASQDIWHYLNWYQQKPGKTVKLLIYYTSQLHSGVPSRFS |
| | GSGSGTDYTLTISSLQPEDFATYFCQQGDTLPWTFGQGTKLEIK |
| 917 | DIQMTQSPSSLSASVGDRVTITCRASQDIWNYLNWYQQKPGKAVKLLIYYTSILETGVPSRFS |
| | GSGSGTDYTLTISSLQPEDFATYFCQQGDSLPWTFGQGTKLEIK |
| 918 | DIQMTQSPSSLSASVGDRVTITCRASQDIWNYLNWYQQKPGKAVKLLIYYTSGLETGVPSRFS |
| | GSGSGTDYTLTISSLQPEDFATYFCQQGDMLPWTFGQGTKLEIK |
| 919 | DIQMTQSPSSLSASVGDRVTITCRASQDIWNYLNWYQQKPGKAVKLLIYYTSMLETGVPSRFS |
| | GSGSGTDYTLTISSLQPEDFATYFCQQGDALPWTFGQGTKLEIK |
| 920 | DIQMTQSPSSLSASVGDRVTITCRASQDIWNYLNWYQQKPGKAVKLLIYYTSGLHSGVPSRFS |
| | GSGSGTDYTLTISSLQPEDFATYFCQQGDTLPWTFGQGTKLEIK |

TABLE 21
20E6 VH variants isolated from TRIM library after
2 hour off-rate selection, P1 population

| Seq | |
|---|---|
| ID | |
| NO. | Amino acid sequence |
| 921 | EVQLVQSGAEVKKPGASVKVSCKASGYTFTSYDVHWVRQAPGQGLEWMGRIDPQSGGVKYAQK |
| | FQGRATLTVDTSTSTAYMELSRLRSDDTAVYYCARWDYGGYFDVWGQGTLVTVSS |
| 922 | EVQLVQSGAEVKKPGASVKVSCKASGYTFTSYDMHWVRQAPGQGLEWMGRIDPQSGGIKYAQK |
| | FQGRATLTVDTSTSTAYMELSRLRSDDTAVYYCARWDYGGYFDVWGQGTLVTVSS |
| 923 | EVQLVQSGAEVKKPGASVKVSCKASGYTFTSYDMHWVRQAPGQGLEWMGRIDPQTGGTKYAQK |
| | FQGRATLTVDTSTSTAYMELSRLRSDDTAVYYCARWDYGGYFDVWGQGTLVTVSS |
| 924 | EVQLVQSGAEVKKPGASVKVSCKASGYTFTNYYMAWVRQAPGQGLEWMGRIDPQDGAIKYAQK |
| | FQGRATLTVDTSTSTAYMELSRLRSDDTAVYYCARWDYGGYFDVWGQGTLVTVSS |
| 925 | EVQLVQSGAEVKKPGASVKVSCKASGYTETRYDMHWVRQAPGQGLEWMGRIDPTSGGIKYAQK |
| | FQGRATLTVDTSTSTAYMELSRLRSDDTAVYYCARWDYGGYFDVWGQGTLVTVSS |
| 926 | EVQLVQSGAEVKKPGASVKVSCKASGYTFTSYDKHWVRQAPGQGLEWMGRIDPQDGGIKYAQK |
| | FQGRATLTVDTSTSTAYMELSRLRSDDTAVYYCARWDYGGYFDVWGQGTLVTVSS |
| 927 | EVQLVQSGAEVKKPGASVKVSCKASGYTFTRYDLHWVRQAPGQGLEWMGRIDPQSGGTKYAQK |
| | FQGRATLTVDTSTSTAYMELSRLRSDDTAVYYCARWDYGGQFDVWGQGTLVTVSS |
| 928 | EVQLVQSGAEVKKPGASVKVSCKASGYTFTYYDMHWVRQAPGQGLEWMGRIDPYDGDIKYAQK |
| | FQGRATLTVDTSTSTAYMELSRLRSDDTAVYYCARWDYGGYFDVWGQGTLVTVSS |
| 929 | EVQLVQSGAEVKKPGASVKVSCKASGYTFTFYDMHWVRQAPGQGLEWMGRIDPDRGGIKYAQK |
| | FOGGATLTVDTSTSTAYMELSRLRSDDTAVYYCARWDYGGYFDVWGQGTLVTVSS |
| 930 | EVQLVQSGAEVKKPGASVKVSCKASGYTFTSYDMHWVRQAPGQGLEWMGRIDPQTGGEKYAQK |
| | FQGRATLTVDTSTSTAYMELSRLRSDDTAVYYCARWAYGGQFDVWGQGTLVTVSS |
| 931 | EVQLVQSGAEVKKPGASVKVSCKASGYTFTRYDLHWVRQAPGQGLEWMGRIDPDDGGRKYAQK |
| | FQGRATLTVDTSTSTAYMELSRLRSDDTAVYYCARWDYGGYFDVWGQGTLVTVSS |
| 932 | EVQLVQSGAEVKKPGASVKVSCKASGYTFTSYDMHWVRQAPGQGLEWMGRIDPQNGGDKYAQK |
| | FQGRATLTVDTSTSTAYMELSRLRSDDTAVYYCARWDYGGYFDVWGQGTLVTVSS |
| 933 | EVQMVQSGAEVKKPGASVKVSCKASGYTFTSYDLHWVRQAPGQGLEWMGRIDPDSGGVKYAQK |
| | FQGRATLTVDTSTSTAYMELSRLRSDDTAVYYCARWDYGGYFDVWGQGTLVTVSS |
| 934 | EVQLVQSGAEVKKPGASVKVSCKASGYTFTRYDMHWVRQAPGQGLEWMGRIDPQTGGVKYAQK |
| | FQGRATLTVDTSTSTAYMELSRLRSDDTAVYYCARWDYGGYFDVWGQGTLVTVSS |
| 935 | EVQLVQSGAEVKKPGASVKVSCKASGYTFTSYDMHWVRQAPGQGLEWMGRIDPQSGGIRYAQK |
| | FQGRATLTVDTSTSTAYMELSRLRSDDTAVYYCARWDYGGYFDVWGQGTLVTVSS |
| 936 | EVQLVQSGAEVKKPGASVKVSCKASGYTFTSYDMHWVRQAPGQGLEWMGRIDPQSGGIRYAQK |
| | FQGRATLTVDTSTSTAYMELSRLRSDDTAVYYCARWDYGGYFDVWGQGTLVTVSS |
| 937 | EVQLVQSGAEVKKPGASVKVSCKASGYTFTSYWMQWVRQAPGQGLEWMGRIDPQDGDIKYAQK |
| | FQGRATLTVDTSTSTAYMELSRLRSDDTAVYYCARYDYGGYFDVWGQGTLVTVSS |
| 938 | EVQLVQSGAEVKKPGASVKVSCKASGYTFTSYDMHWVRQAPGQGLEWMGRIDPQSGGSKYAQK |
| | FQGRATLTVDTSTSTAYMELSRLRSDDTAVYYCARWDYGGYFDVWGQGTLVTVSS |
| 939 | EVQLVQSGAEVKKPGASVKVSCKASGYTFTSYYMQWVRQAPGQGLEWMGRIDPFDGDIKYAQK |
| | FQGRATLTVDTSTSTAYMELSRLRSDDTAVYYCARWDYGGYFDVWGQGTLVTVSS |
| 940 | EVQLVQSGAEVKKPGASVKVSCKASGYTFTKYDMHWVRQAPGQGLEWMGRIDPQDGGFKYAQK |
| | FQGRATLTVDTSTSTAYMELSRLRSDDTAVYYCARWDYGGYFDVWGQGTLVTVSS |
| 941 | EVQLVQSGAEVKKPGASVKVSCKASGYTFTYYDMHWVRQAPGQGLEWMGRIDPQSGGIKYAQK |
| | FQGRATLTVDTSTSTAYMELSRLRSDDTAVYYCARWDYGGYFDVWGQGTLVTVSS |
| 942 | EVQLVQSGAEVKKPGASVKVSCKASGYTFTSYDLHWVRQAPGQGLEWMGRIDPFNGGGKYAQK |
| | FQGRATLTVDTSTSTAYMELSRLRSDDTAVYYCARWDYGGYFDVWGQGTLVTVSS |
| 943 | EVQLVQSGAEVKKPGASVKVSCKASGYTFTSYDSHWVRQAPGQGLEWMGRIDPQTGGIKYAQK |
| | FQGRATLTVDTSTSTAYMELSRLRSDDTAVYYCARWDYGGYFDVWGQGTLVTVSS |
| 944 | EVQLVQSGAEVKKPGASVKVSCKASGYTFTRYDMHWVRQAPGQGLEWMGRIDPQTGGIKYAQK |
| | FQGRATLTVDTSTSTAYMELSRLRSDDTAVYYCARWDYGGYFDVWGQGTLVTVSS |
| 945 | EVQLVQSGAEVKKPGASVKVSCKASGYTFTSYDMHWVRQAPGQGLEWMGRIDPQSGGTKYAQK |
| | FQGRATLTVDTSTSTAYMELSRLRSDDTAVYYCARWDYGGYFDVWGQGTLVTVSS |
| 946 | EVQLVQSGAEVKKPGASVKVSCKASGYTFTSYDMHWVRQAPGQGLEWMGRIDPQDGGIRYAQK |
| | FQGRATLTVDTSTSTAYMELSRLRSDDTAVYYCARWDYGGYFDVWGQGTLVTVSS |
| 947 | EVQLVQSGAEVKKPGASVKVSCKASGYTFTSYDMHWVRQAPGQGLEWMGRIDPQSGGVKYAQK |
| | FQGRATLTVDTSTSTAYMELSRLRSDDTAVYYCARWDYGGYFDVWGQGTLVTVSS |
| 948 | EVQLVQSGAEVKKPGASVKVSCKASGYTFTNYDMHWVRQAPGQGLEWMGRIDPQNGGVKYAQK |
| | FQGRATLTVDTSTSTAYMELSRLRSDDTAVYYCARWDYGGYFDVWGQGTLVTVSS |
| 949 | EVQLVQSGAEVKKPGASVKVSCKASGYTFTSYDMHWVRQAPGQGLEWMGRIDPQSGGSRYAQK |
| | FQGRATLTVDTSTSTAYMELSRLRSDDTAVYYCARWDFGGYFDVWGQGTLVTVSS |
| 950 | EVQLVQSGAEVKKPGASVKVSCKASGYTFTRYDMHWVRQAPGQGLEWMGRIDPQSGGIKYAQK |
| | FQGRATLTVDTSTSTAYMELSRLRSDDTAVYYCARWDYGGYFDVWGQGTLVTVSS |

TABLE 22
20E6 VL variants isolated from TRIM library after
2 hour off-rate selection, P1 population

| Seq | |
|---|---|
| ID | Amino acid sequence |
| 951 | DIQMTQSPSSLSASVGDRVTITCRASQDIYNYLNWYQQKPGKAVKLLIYYTSLLHSGVPSRFS |
| | GSGSGTDYTLTISSLQPEDFATYFCQQGDEVPWTFGQGTKLEIK |
| 952 | DIQMTQSPSSLSASVGDRVTITCRASQDIYNYLNWYQQKPGKAVKLLIYYTSGLHSGVPSRFS |
| | GSGSGTDYTLTISSLOPEDFATYFCQQGDALPWTFGQGTKLEIK |
| 953 | DIQMTQSPSSLSASVGDRVTITCRASQDIFNYLNWYQQKPGKAVKLLIYYTSLLHSGVPSRFS |
| | GSGSGTDYTLTISSLQPEDFATYFCQQGDLLPWTFGQGTKLEIK |
| 954 | DIQMTQSPSSLSASVGDRVTITCRASQDIYNYLNWYQQKPGKAVKLLIYYTSVLHSGVPSRFS |
| | GSGSGTDYTLTISSLOPEDFATYFCQQGDELPWTFGQGTKLEIK |
| 955 | DIQMTQSPSSLSASVGDRVTITCRASQDIYNYLNWYQQKPGKAVKLLIYYTSVLHSGVPSRFS |
| | GSGSGTDYTLTISSLQPEDFATYFCQQGDTLPWTFGQGTKLEIK |
| 956 | DIQMTQSPSSLSASVGDRVTITCRASQDIFNYLNWYQQKPGKAVKLLIYYTSILHSGVPSRFS |
| | GSGSGTDYTLTISSLQPEDFATYFCQQGDELPWTFGQGTKLEIK |
| 957 | DIQMTQSPSSLSASVGDRVTITCRASQDIYNYLNWYQQKPGKAVKLLIYYTSNLHSGVPSRFS |
| | GSGSGTDYTLTISSLQPEDFATYFCQQGDELPWTFGQGTKLEIK |
| 958 | DIQMTQSPSSLSASVGDRVTITCRASQDIYYYLNWYQQKPGKAVKLLIYYTSILHSGVPSRFS |
| | GSGSGTDYTLTISSLQPEDFATYFCQQGDTLPWTFGQGTKLEIK |
| 959 | DIQMTQSPSSLSASVGDRVTITCRASQDIFNYLNWYQQKPGKAVKLLIYYTSYLHSGVPSRFS |
| | GSGSGTDYTLTISSLQPEDFATYFCQQGDELPWTFGQGTKLEIK |
| 960 | DIQMTQSPSSLSASVGDRVTITCRASQDIYNYLNWYQQKPGKAVKLLIYYTSHLHSGVPSRFS |
| | GSGSGTDYTLTISSLQPEDFATYFCQQGDELPWTFGQGTKLEIK |
| 961 | DIQMTQSPSSLSASVGDRVTITCRASQDIYNYLNWYQQKPGKAVKLLIYYTSVLHSGVPSRFS |
| | GSGSGTDYTLTISSLQPEDFATYFCQQGDALPWTFGQGTKLEIK |
| 962 | DIQMTQSPSSLSASVGDRVTITCRASQDIYNYLNWYQQKPGKAVKLLIYYTSQLHSGVPSRES |
| | GSGSGTDYTLTISSLOPEDFATYFCQQGDTLPWTFGQGTKLEIK |
| 963 | DIQMTQSPSSLSASVGDRVTITCRASQDIYKYLNWYQQKPGKAVKLLIYYTSGLHSGVPSRFS |
| | GSGSGTDYTLTISSLOPEDFATYFCQQGDTLPWTFGQGTKLEIK |
| 964 | DIQMTQSPSSLSASVGDRVTITCRASQDIYNYLNWYQQKPGKAVKLLIYYTSLLHSGVPSRFS |
| | GSGSGTDYTLTISSLOPEDFATYFCQQGDELPWTFGQGTKLEIK |
| 965 | DIQMTQSPSSLSASVGDRVTITCKASQDIYNYLNWYQQKPGKAVKLLIYYTSHLHSGVPSRFS |
| | GSGSGTDYTLTISSLQPEDFATYFCQQGDTLPWTFGQGTKLEIK |
| 966 | DIQMTQSPSSLSASVGDRVTITCRASQDIFNYLNWYQQKPGKAVKLLIYYTSILHSGVPSRFS |
| | GSGSGTDYTLTISSLQPEDFATYFCQQGDTLPWTFGQGTKLEIK |
| 967 | DIQMTQSPSSLSASVGDRVTITCRASQDIYNYLNWYQQKPGKAVKLLIYYTSGLHSGVPSRFS |
| | GSGSGTDYTLTISSLQPEDFATYFCQQGDYLPWTFGQGTKLEIK |
| 968 | DIQMTQSPSSLSASVGDRVTITCRASQDIYNYLNWYQQKPGKAVKLLIYYTSILHSGVPSRFS |
| | GSGSGTDYTLTISSLQPEDFATYFCQQGDELPWTFGQGTKLEIK |
| 969 | DIQMTQSPSSLSASVGDRVTITCRASQDIYNYLNWYQQKPGKAVKLLIYYTSFLHSGVPSRFS |
| | GSGSGTDYTLTISSLQPEDFATYFCQQGDALPWTFGQGTKLEIK |
| 970 | DIQMTQSPSSLSASVGDRVTITCRASQDIYNYLNWYQQKPGKAVKLLIYYTSILHSGVPSRFS |
| | GSGYGTDYTLTISSLQPEDFATYFCQQGDTLPWTFGQGTKLEIK |

TABLE 23
20E6 VH variants isolated from TRIM library after
2 hour off-rate selection, P2 population

| Seq | |
|---|---|
| ID | |
| NO. | Amino acid sequence |
| 971 | EVQLVQSGAEVKKPGASVKVSCKASGYTFTRYDLHWVRQAPGQGLEWMGRIDPQNGGIKYAQK |
| | FQGRATLTVDTSTSTAYMELSRLRSDDTAVYYCARWDYGGYFDVWGQGTLVTVSS |
| 972 | EVQLVOSGAEVKKPGASVKVSCKASGYTFTYYYMNWVRQAPGQGLEWMGRIDPISGGTKYAQK |
| | FQGRATLTVDTSTSTAYMELSRLRSDDTAVYYCARYDHGGYFDVWGQGTLVTVSS |
| 973 | EVQLVQSGAEVKKPGASVKVSCKASGYTFTSYDLHWVRQAPGQGLEWMGRIDPQSGGIKYAQK |
| | FQGRATLTVDTSTSTAYMELSRLRSDDTAVYYCARWDYGGHFDVWGQGTLVTVSS |
| 974 | EVQLVQSGAEVKKPGASVKVSCKASGYTETSYDMHWVRQAPGQGLEWMGRIDPQTGGVRYAQK |
| | FQGRATLTVDTSTSTAYMELSRLRSDDTAVYYCARWDYGGYFDVWGQGTLVTVSS |
| 975 | EVQLVQSGAEVKKPGASVKVSCKASGYTFTSYDLHWVRQAPGQGLEWMGRIDPQDGGIKYAQK |
| | FQGRATLTVDTSTSTAYMELSRLRSDDTAVYYCARWDYGGYFDVWGQGTLVTVSS |
| 976 | EVQLVQSGAEVKKPGASVKVSCKASGYTFTAYDLHWVRQAPGQGLEWMGRIDPDDGGIRYAQK |
| | FQGRATLTVDTSTSTAYMELSRLRSDDTAVYYCARWDYGGYFDVWGQGTLVTVSS |
| 977 | EVQLVQSGAEVKKPGASVKVSCKASGYTFTSYDMHWVRQAPGQGLEWMGRIDPQSGGIRYAQK |
| | FQGRATLTVDTSTSTAYMELSRLRSDDTAVYYCARWDYGGYFDVWGQGTLVTVSS |
| 978 | EVQLVQSGAEVKKPGASVKVSCKASGYTFTSYDMHWVRQAPGQGLEWMGRIDPQDGDVKYAQK |
| | FQGRATLTVDTSTSTAYMELSRLRSDDTAVYYCARWDYGGYFDVWGQGTLVTVSS |
| 979 | EVQLVQSGAEVKKPGASVKVSCKASGYTFTSYDMHWVRQAPGQGLEWMGRIDPQTGDIKYAQK |
| | FQGRATLTVDTSTSTAYMELSRLRSDDTAVYYCARWDYGGQFDVWGQGTLVTVSS |
| 980 | EVQLVQSGAEVKKPGASVKVSCKASGYTETYYDMHWVRQAPGQGLEWMGRIDPQSGGIIYAQK |
| | FQGRATLTVDTSTSTAYMELSRLRSDDTAVYYCARWDYGGYFDVWGQGTLVTVSS |
| 981 | EVQLVQSGAEVKKPGASVKVSCKASGYTFTSYDMHWVRQAPGQGLEWMGRIDPQSGGVRYAQK |
| | FQGRATLTVDTSTSTAYMELSRLRSDDTAVYYCARWDYGGYFDVWGQGTLVTVSS |
| 982 | EVQLVQSGAEVKKPGASVKVSCKASGYTFTYYDMHWVRQAPGQGLEWMGRIDPQSGGDRYAQK |
| | FQGRATLTVDTSTSTAYMELSRLRSDDTAVYYCARWDYGGYFDVWGQGTLVTVSS |
| 983 | EVQLVQSGAEVKKPGASVKVSCKASGYTFTSYDMHWVRQAPGQGLEWMGRIDPVSGGIKYAQK |
| | FQGRATLTVDTSTSTAYMELSRLRSDDTAVYYCARWDYGGYFDVWGQGTLVTVSS |
| 984 | EVQLVQSGAEVKKPGASVKVSCKASGYTFTSYDMHWVRQAPGQGLEWMGRIDPYSGGIKYAQK |
| | FQGRATLTVDTSTSTAYMELSRLRSDDTTVYYCARWDYGGQFDVWGQGTLVTVSS |
| 985 | EVQLVQSGAEVKKPGASVKVSCKASGYTFTYYDVHWVRQAPGQGLEWMGRIDPQSGGGRYAQK |
| | FQGRATLTVDTSTSTAYMELSRLRSDDTAVYYCARWDYGGYFDVWGQGTLVTVSS |
| 986 | EVQLVQSGAEVKKPGASVKVSCKASGYTFTKYDMHWVRQAPGQGLEWMGRIDPQTGGVKYAQK |
| | FQGRATLTVDTSTSTAYMELSRLRSDDTAVYYCARWDYGGYFDVWGQGTLVTVSS |
| 987 | EVQLVQSGAEVKKPGASVKVSCKASGYTFTSYWMQWVRQAPGQGLEWMGRIDPQDGDIKYAQK |
| | FQGRATLTVDTSTSTAYMELSRLRSDDTAVYYCARWDYGGYFDVWGQGTLVTVSS |
| 988 | EVQLVQSGAEVKKPGASVKVSCKASGYTFTSYDMHWVRQAPGQGLEWMGRIDPESGGTKYAQK |
| | FQGRATLTVDTSTSTAYMELSRLRSDDTAVYYCARWDYGGYFDVWGQGTLVTVSS |
| 989 | EVQLVQSGAEVKKPGASVKVSCKASGYTFTSYDMHWVRQAPGQGLEWMGRIDPQSGGIKYAQK |
| | FQGRATLTVDTSTSTAYMELSRLRSDDTAVYYCARWHTGGQFDVWGQGTLVTVSS |
| 990 | EVQLVQSGAEVKKPGASVKVSCKASGYTFTSYDMHWVRQAPGQGLEWMGRIDPQSGGTKYAQK |
| | FQGRATLTVDTSTSTAYMELSRLRSDDTAVYYCARWDYGGYFDVWGQGTLVTVSS |
| 991 | EVQLVQSGAEVKKPGASVKVSCKASGYTFTRYDLHWVRQAPGQGLEWMGRIDPPSGGVLYAQK |
| | FQGRATLTVDTSTSTAYMELSRLRSDDTAVYYCARWDYGGYFDVWGQGTLVTVSS |
| 992 | EVQLVQSGAEVKKPGASVKVSCKASGYTFTSYDMHWVRQAPGQGLGWMGRIDPQSGGIKYAQK |
| | FQGRATLTVDTSTSTAYMELSRLRSDDTAVYYCARWDYGGFFDVWGQGTLVTVSS |
| 993 | EVQLVQSGAEVKKPGASVKVSCKASGYTFTQYDMHWVRQAPGQGLEWMGRIDPQSGGIKYAQK |
| | FQGRATLTVDTSTSTAYMELSRLRSDDTAVYYCARYDYGGYFDVWGQGTLVTVSS |
| 994 | EVQLVQSGAEVKKPGASVKVSCKASGYTFTEYDMHWVRQAPGQGLEWMGRIDPQSGGQKYAQK |
| | FQGRATLTVDTSTSTAYMELSRLRSDDTAVYYCARWDYGGFFDVWGQGTLATVSS |
| 995 | EVQLVQSGAEVKKPGASVKVSCKASGYTFTNYDMHWVRQAPGQGLEWMGRIDPQVGGIKYAQK |
| | FQGRATLTVDTSTSTAYMELSRLRSDDTAVYYCARWDYGGYFDVWGQGTLVTVSS |
| 996 | EVQLVQSGAEVKKPGASVKVSCKASGYTFTHYFMNWVRQAPGQGLEWMGRIDPQSGSIRYAQK |
| | FQGRATLTVDTSTSTAYMELSRLRSDDTAVYYCARWDYGGYFDVWGQGTLVTVSS |
| 997 | EVQLVQSGAEVKKPGASVKVSCKASGYTFTSYDMHWVRQAPGQGLEWMGRIDPNSGGIKYAQK |
| | FQGRATLTVDTSTSTAYMELSRLRSDDTAVYYCARWDYGGYFDVWGQGTLVTVSS |
| 998 | EVQLVQSGAEVKKPGASVKVSCKASGYTFTYYDLHWVRQAPGQGLEWMGRIDPQSGGIKYAQK |
| | FQGRATLTVDTSTSTAYMELSRLRSDDTAVYYCARWDYGGYFDVWGQGTLVTVSS |
| 999 | EVQLVQSGAEVKKPGASVKVSCKASGYTFTSYDMHWVRQAPGQGLEWMGRIDPQSGGQKYAQK |
| | FQGRATLTVDTSTSTAYMELSRLRSDDTAVYYCARWDYGGYFDVWGQGTLVTVSS |
| 1000 | EVRLVQSGAEVKKPGASVKVSCKASGYTFTSYDMHWVRQAPGQGLEWMGRIDPQSGGVKYAQK |
| | FQGRATLTVDTSTSTAYMELSRLRSDDTAVYYCARWDYGGYFDVWGQGTLVTVSS |
| 1001 | EVQLVQSGAEVKKPGASVKVSCKASGYTFTSYDTHWVRQAPGQGLEWMGRIDPQSGGIKYAQK |
| | FQGRATLTVDTSTSTAYMELSRLRSDDTAVYYCARWDYGGYFDVWGQGTLVTVSS |
| 1002 | EVQLVQSGAEVKKPGASVKVSCKASGYTFTSYDMHWVRQAPGQGLEWMGRIDPQSGGIKYAQK |
| | FQGRATLTVDTSTSTAYMELSRLRSDDTAVYYCARWDYGGYFDVWGQGTLVTVSS |
| 1003 | EVQLVQSGAEVKKPGASVKVSCKASGYTFTQYDMHWVRQAPGQGLEWMGRIDPQWGGIKYAQK |
| | FQGRATLTVDTSTSTAYMELSRLRSDDTAVYYCARWDYGGYFDVWGQGTLVTVSS |
| 1004 | EVQLVQSGAEVKKPGASVKVSCKASGYTFTLYDMHWVRQAPGQGLEWMGRIDPQSGGIKYAQK |
| | FQGRATLTVDTSTSTAYMELSRLRSDDTAVYYCARWDYGGYFDVWGQGTLVTVSS |
| 1005 | EVQLVQSGAEVKKPGASVKVSCKASGYTFTYYDMHWVRQAPGQGLEWMGRIDPQSGGIKYAQK |
| | FQGRATLTVDTSTSTAYMELSRLRSDDTAVYYCARWDYGGYFDVWGQGTLVTVSS |
| 1006 | EVQLVQSGAEVKKPGASVKVSCKASGYTFTSYDMHWVRQAPGQGLEWMGRIDPQSGGIRYAQK |
| | FQGRATLTVDTSTSTAYMELSRLRSDDTAVYYCARYDYGGYFDVWGQGTLVTVSS |
| 1007 | EVQLVQSGAEVKKPGASVKVSCKASGYTFTSYDIHWVRQAPGQGLEWMGRIDPQSGGIKYAQK |
| | FQGRATLTVDTSTSTAYMELSRLRSDDTAVYYCARWDYGGYFDVWGQGTLVTVSS |
| 1008 | EVQLVQSGAEVKKPGASVKVSCKASGYTFTSYDMHWVRQAPGQGLEWMGRIDPQSGGIKYAQK |
| | FQGRATLTVDTSTSTAYMELSRLRSDDTAVYYCARWDYGGYFDVWGQGTLVTVSS |
| 1009 | EVQLVQSGAEVKKPGASVKVSCKASGYTFTRYDMHWVRQAPGQGLEWMGRIDPKSGGIKYAQK |
| | FQGRATLTVDTSTSTAYMELSRLRSDDTAVYYCARWDYGGYFDVWGQGTLVTVSS |

TABLE 24
20E6 VL variants isolated from TRIM library after
2 hour off-rate selection, P2 population

| Seq | |
|---|---|
| ID | |
| NO. | Amino acid sequence |
| 1010 | DIQMTQSPSSLSASVGDRVTITCRASQDIYNYLNWYQQKPGKAVKLLIYYTSVLHSGVPSRF |
| | SGSGSGTDYTLTISSLQPEDFATYFCQQGDTLPWTFGQGTKLEIK |
| 1011 | DIQMTQSPSSLSASVGDRVTITCRASQDIYNYLNWYQQKPGKAVKLLIYYTSGLHSGVPSRF |
| | SGSGSGTDYTLTISSLQPEDFATYFCQQGDTLPWTFGQGTKLEIK |
| 1012 | DIQMTQSPSSLSASVGDRVTITCRASQDIYSYLNWYQQKPGKAVKLLIYYTSLLHSGVPSRF |
| | SGSGSGTDYTLTISSLQPEDFATYFCQQGDLLPWTFGQGTKLEIK |
| 1013 | DIQMTQSPSSLSASVGDRVTITCRASQDIYNYLNWYQQKPGKAVKLLIYYTSVLHSGVPSRF |
| | SGSGSGTDYTLTISSLOPEDFATYFCQQGDELPWTFGQGTKLEIK |
| 1014 | DIQMTQSPSSLSASVGDRVTITCRASQDIFNYLNWYQQKPGKAVKLLIYYTSNLHSGVPSRF |
| | SGSGSGTDYTLTISSLQPEDFATYFCQQGDTLPWTFGQGTKLEIK |
| 1015 | DIQMTQSPSSLSASVGDRVTITCRASQDIFNYLNWYQQKPGKAVKLLIYYTSILHSGVPSRF |
| | SGSGSGTDYTLTISSLQPEDFATYFCQQGDTLPWTFGQGTKLEIK |
| 1016 | DIQMTQSPSSLSASVGDRVTITCRASQDIYNYLNWYQQKPGKAVKLLIYYTSLLHSGVPSRF |
| | SGSGSGTDYTLTISSLQPEDFATYFCQQGDELPWTFGQGTKLEIK |
| 1017 | DIQMTQSPSSLSASVGDRVTITCRASQDIYNYLNWYQQKPGKAVKLLIYYTSQLHSGVPSRF |
| | SGSGSGTDYTLTISSLQPEDFATYFCQQGDTLPWTFGQGTKLEIK |
| 1018 | DIQMTQSPSSLSASVGDRVTITCRASQDIYNYLNWYQQKPGKAVKLLIYYTSGLHSGVPSRF |
| | SGSGSGTDYTLTISSLOPEDFATYFCQQGDELPWTFGQGTKLEIK |
| 1019 | DIQMTQSPSSLSASVGDRVTITCRASQDIFNYLNWYQQKPGKAVKLLIYYTSVLHSGVPSRF |
| | SGSGSGTDYTLTISSLOPEDFATYFCQQGDELPWTFGQGTKLEIK |
| 1020 | DIQMTQSPSSLSASVGDRVTITCRASQDIYNYLNWYQQKPGKAVKLLIYYTSILHSGVPSRF |
| | SGSGSGTDYTLTISSLQPEDFATYFCQQGDALPWTFGQGTKLEIK |
| 1021 | DIQMTQSPSSLSASVGDRVTITCRASQDIYHYLNWYQQKPGKAVKLLIYYTSELHSGVPSRF |
| | SGSGSGTDYTLTISSLQPEDFATYFCQQGDELPWTFGQGTKLEIK |
| 1022 | DIQMTQSPSSLSASVGDRVTITCRASQDIFNYLNWYQQKPGKAVKLLIYYTSGLHSGVPSRF |
| | SGSGSGTDYTLTISSLQPEDFATYFCQQGDTLPWTFGQGTKLEIK |
| 1023 | DIQMTQSPSSLSASVGDRVTITCRASQDIYNYLNWYQQKPGKAVKLLIYYTSILHSGVPSRF |
| | SGSGSGTDYTLTISSLQPEDFATYFCQQGDGLPWTFGQGTKLEIK |
| 1024 | DIQMTQSPSSLSASVGDRVTITCRASQDIWNYLNWYQQKPGKAVKLLIYYTSRLHSGVPSRF |
| | SGSGSGTDYTLTISSLQPEDFATYFCQQGDTLPWTFGQGTKLEIK |
| 1025 | DIQMTQSPSSLSASVGDRVTITCRASQDIYNYLNWYQQKPGKAVKLLIYYTSLLHSGVPSRF |
| | SGSGSGTDYTLTISSLQPEDFATYFCQQGDTLPWTFGQGTKLDIK |
| 1026 | DIQMTQSPSSLSASVGDRVTITCRASQDIYNYLNWYQQKPGKAVKLLIYYTSILHSGVPSRF |
| | SGSGSGTDYTLTISSLQPEDFATYFCQQGDTLPWTFGQGTKLEIK |
| 1027 | DIQMTQSPSSLSASVGDRVTITCRASQDIYKYLNWYQQKPGKAVKLLIYYTSRLHSGVPSRF |
| | SGSGSGTDYTLTISSLQPEDFATYFCQQGDELPWTFGQGTKLEIK |
| 1028 | DIQMTQSPSSLSASVGDRVTITCRASQDIFNYLNWYQQKPGKAVKLLIYYTSRLHSGVPSRF |
| | SGSGSGTDYTLTISSLQPEDFATYFCQQGDTLPWTFGQGTKLEIK |
| 1029 | DIQMTQSPSSLSASVGDRVTITCRASQDIFNYLNWYQQKPGKAVKLLIYYTSSLHSGVPSRF |
| | SGSGSGTDYTLTISSLQPEDFATYFCQQGDGLPWTFGQGTKLEIK |
| 1030 | DIQMTQSPSSLSASVGDRVTITCRASQDIYNYLNWYQQKPGKAVKLLIYYTSYLHSGVPSRF |
| | SGSGSGTDYTLTISSLQPEDFATYFCQQGDTLPWTFGQGTKLEIK |
| 1031 | DIQMTQSPSSLSASVGDRVTITCRASQDIYNYLNWYQQKPGKAVKLLIYYTSELHSGVPSRF |
| | SGSGSGTDYTLTISSLQPEDFATYFCQQGDTLPWTFGQGTKLEIK |
| 1032 | DIQMTQSPSSLSASVGDRVTITCRASQDIYKYLNWYQQKPGKAVKLLIYYTSHLHSGVPSRF |
| | SGSGSGTDYTLTISSLQPEDFATYFCQQGDTLPWTFGQGTKLEIK |
| 1033 | DIQMTQSPSSLSASVGDRVTITCRASQDIFNYLNWYQQKPGKAVKLLIYYTSRLHSGVPSRE |
| | SGSGSGTDYTLTISSLQPEDFATYFCQQGDELPWTFGQGTKLEIK |
| 1034 | DIQMTQSPSSLSASVGDRVTITCRASQDIYNYLNWYQQKPGKAVKLLIYYTSRLHSGVPSRF |
| | SGSGSGTDYTLTISSLQPEDFATYFCQQGDDIPWTFGQGTKLEIK |
| 1035 | DIQMTQSPSSLSASVGDRVTITCRASQDIYNYLNWYQQKPGKAVKLLIYYTSRLHSGVPSRF |
| | SGSGSGTDYTLTISSLQPEDFATYFCQQGDELPWTFGQGTKLEIK |
| 1036 | DIQMTQSPSSLSASVGDRVTITCRASQDIYNYLNWYQQKPGKAVKLLIYYTSRLHSGVPSRF |
| | SGSGSGTDYTLTISSLQPEDFATYFCQQGDTLPWTFGQGTKLEIK |

TABLE 25
20E6 VH variants isolated from TRIM library after
2 hour off-rate selection, P3 population

| Seq | |
|---|---|
| ID | |
| NO. | Amino acid sequence |
| 1037 | EVQLVQSGAEVKKPGASVKVSCKASGYTFTSYDMHWVRQAPGQGLEWMGRIDPQSGDIKYAQK |
| | FQGRATLTVDTSTSTAYMELSRLRSDDTAVYYCARYDHGGYFDVWGQGTLVTVSS |
| 1038 | EVQLVQSGAEVKKPGASVKVSCKASGYTFTKYDIHWVRQAPGQGLEWMGRIDPQSGPIKYAQK |
| | FQGRATLTVDTSTSTAYMELSRLRSDDTAVYYCARWDYGGYFDVWGQGTLVTVSS |
| 1039 | EVQLVOSGAEVKKPGASVKVSCKASGYTFTSYDMHWVRQAPGQGLEWMGRIDPQSGGIKYAQK |
| | FQGRATLTVDTSTSTAYMELSRLRSDDTAVYYCARWSQGGYFDVWGQGTLVTVSS |
| 1040 | EVQLVQSGAEVKKPGASVKVSCKASGYTFTSYDMHWVRQAPGQGLEWMGRIDPQSGGIKYAQK |
| | FQGRATLTVDTSTSTAYMELSRLRSDDTAVYYCARYDHGGYFDVWGQGTLVTVSS |
| 1041 | EVQLVQSGAEVKKPGASVKVSCKASGYTFTQYDMHWVRQAPGQGLEWMGRIDPANGGIKYAQK |
| | FQGRATLTVDTSTSTAYMELSRLRSDDTAVYYCARYDYGGYFDVWGQGTLVTVSS |
| 1042 | EVQLVQSGAEVKKPGASVKVSCKASGYTFTYYDLHWVRQAPGQGLEWMGRIDPLSGGGVYAQK |
| | FQGRATLTVDTSTSTAYMELSRLRSDDTAVYYCARYDHGGYFDVWGQGTLVTVSS |
| 1043 | EVQLVQSGAEVKKPGASVKVSCKASGYTFTFYDIHWVRQAPGQGLEWMGRIDPQSGGIKYAQK |
| | FQGRATLTVDTSTSTAYMELSRLRSDDTAVYYCARWDYGGYFDVWGQGTLVTVSS |
| 1044 | EVQLVQSGAEVKKPGASVKVSCKASGYTFTSYDLHWVRQAPGQGLEWMGRIDPQSGGHKYAQK |
| | FQGRATLTVDTSTSTAYMELSRLRSDDTAVYYCARYDFGGYFDVWGQGTLVTVSS |
| 1045 | EVQLVQSGAEVKKPGASVKVSCKASGYTFTSYDIHWVRQAPGQGLEWMGRIDPYSGGIKYAQK |
| | FQGRATLTVDTSTSTAYMELSRLRSDDTAVYYCARWDYGGYFDVWGQGTLVTVSS |
| 1046 | EVQLVQSGAEVKKPGASVKVSCKASGYTFTSYWMHWVRQAPGQGLEWMGRIDPQSGGIVYAQK |
| | FQGRATLTVDTSTSTAYMELSRLRSDDTAVYYCARWDYGGYFDVWGQGTLVTVSS |
| 1047 | EVQLVQSGAEVKKPGASVKVSCKASGYTFTIYDMHWVRQAPGQGLEWMGRIDPQSGGIKYAQK |
| | FQGRATLTVDTSTSTAYMELSRLRSDDTAVYYCARWEYGGYFDVWGQGTLVTVSS |
| 1048 | EVQLVOSGAEVKKPGASVKVSCKASGYTFTSYDLHWVRQAPGQGLEWMGRIDPQSGGIKYAQK |
| | FQGRATLTVDTSTSTAYMELSRLRSDDTAVYYCARWDQGGYFDVWGQGTLVTVSS |
| 1049 | EVQLVQSGAEVKKPGASVKVSCKASGYTFTSYDINWVRQAPGQGLEWMGRIDPQSGDYKYAQK |
| | FQGRATLTVDTSTSTAYMELSRLRSDDTAVYYCARYDHGGYFDVWGQGTLVTVSS |
| 1050 | EVQLVQSGAEVKKPGASVKVSCKASGYTFTSYDMHWVRQAPGQGLEWMGRIDPQSGGEKYAQK |
| | FQGRATLTVDTSTSTAYMELSRLRSDDTAVYYCARYDYGGYFDVWGQGTLVTVSS |
| 1051 | EVQLVQSGAEVKKPGASVKVSCKASGYTFTSYDMHWVRQAPGQGLEWMGRIDPQTGGIKYAQK |
| | FQGRATLTVDTSTSTAYMELSRLRSDDTAVYYCARYDHGGYFDVWGQGTLVTVSS |
| 1052 | EVQLVQSGAEVKKPGASVKVSCKASGYTFTSYPLHWVRQAPGQGLEWMGRIDPQSGGDIYAQK |
| | FQGRATLTVDTSTSTAYMELSRLRSDDTAVYYCARWDYGGYFDVWGQGTLVTVSS |
| 1053 | EVQLVQSGAEVKKPGASVKVSCKASGYTFTSYWMHWVRQAPGQGLEWMGRIDPQNGGIKYAQK |
| | FQGRATLTVDTSTSTAYMELSRLRSDDTAVYYCARWDYGGYFDVWGQGTLVTVSS |
| 1054 | EVQLVQSGAEVKKPGASVKVSCKASGYTFTSYLLQWVRQAPGQGLEWMGRIDPTSGGDDYAQK |
| | FQGRATLTVDTSTSTAYMELSRLRSDDTAVYYCARWDYGGYFDVWGQGTLVTVSS |
| 1055 | EVQLVQSGAEVKKPGASVKVSCKASGYTFTLYDVHWVRQAPGQGLEWMGRIDPQSGGDKYAQK |
| | FQGRATLTVDTSTSTAYMELSRLRSDDTAVYYCARYDYGGYFDVWGQGTLVTVSS |
| 1056 | EVQLVQSGAEVKKPGASVKVSCKASGYTFTSYDIHWVRQAPGQGLEWMGRIDPQSGGVKYAQK |
| | FQGRATLTVDTSTSTAYMELSRLRSDDTAVYYCARYDYGGYFDVWGQGTLVTVSS |
| 1057 | EVQLVQSGAEVKKPGASVKVSCKASGYTFTSYDMHWVRQAPGQGLEWMGRIDPQSGGSKYAQK |
| | FQGRATLTVDTSTSTAYMELSRLRSDDTAVYYCARYDYGGYFDVWGQGTLVTVSS |
| 1058 | EVQLVQSGAEVKKPGASVKVSCKASGYTFTSYDISWVRQAPGQGLEWMGRIDPQSGGIKYAQK |
| | FQGRATLTVDTSTSTAYMELSRLRSDDTAVYYCARWDYGGYFDVWGQGTLVTVSS |
| 1059 | EVQLVQSGAEVKKPGASVKVSCKASGYTFTSYDMSWVRQAPGQGLEWMGRIDPQGGGTRYAQK |
| | FQGRATLTVDTSTSTAYMELSRLRSDDTAVYYCARWDYAGYFDVWGQGTLVTVSS |
| 1060 | EVQLVQSGAEVKKPGASVKVSCKASGYTFTSYDMHWVRQAPGQGLEWMGRIDPQTGGIKYAQK |
| | FQGRATLTVDTSTSTAYMELSRLRSDDTAVYYCARWDHGGYFDVWGQGTLVTVSS |
| 1061 | EVQLVQSGAEVKKPGASVKVSCKASGYTFTSYDMHWVRQAPGQGLEWMGRIDPDNGGTKYAQK |
| | FQGRATLTVDTSTSTAYMELSRLRSDDTAVYYCARYDHGGYFDVWGQGTLVTVSS |
| 1062 | EVQLVQSGAEVKKPGASVKVSCKASGYTFTSYDVHWVRQAPGQGLEWMGRIDPQSGGILYAQK |
| | FQGRATLTVDTSTSTAYMELSRLRSDDTAVYYCARWEYGGNFDVWGQGTLVTVSS |
| 1063 | EVQLVQSGAEVKKPGASVKVSCKASGYTETHYDSHWVRQAPGQGLEWMGRIDPQSGGIKYAQK |
| | FQGRATLTVDTSTSTAYMELSRLRSDDTAVYYCARYDYGGYFDVWGQGTLVTVSS |
| 1064 | EVQLVQSGAEVKKPGASVKVSCKASGYTFTNYDIHWVRQAPGQGLEWMGRIDPPSGGTKYAQK |
| | FQGRATLTVDTSTSTAYMELSRLRSDDTAVYYCARYDHGGYFDVWGQGTLVTVSS |
| 1065 | EVQLVQSGAEVKKPGASVKVSCKASGYTFTSYDMHWVRQAPGQGLEWMGRIDPQSGGTKYAQK |
| | FQGRATLTVDTSTSTAYMELSRLRSDDTAVYYCARYDHGGYFDVWGQGTLVTVSS |
| 1066 | EVQLVQSGAEVKKPGASVKVSCKASGYTFTTYWMHWVRQAPGQGLEWMGRIDPQSGGSKYAQK |
| | FQGRATLTVDTSTSTAYMELSRLRSDDTAVYYCARWDYGGYFDVWGQGTLVTVSS |
| 1067 | EVQLVQSGAEVKKPGASVKVSCKASGYTFTSYDIHWVRQAPGQGLEWMGRIDPQSGGIKYAQK |
| | FQGRATLTVDTSTSTAYMELSRLRSDDTAVYYCARWDYGGYFDVWGQGTLVTVSS |

TABLE 26
20E6 VL variants isolated from TRIM library after
2 hour off-rate selection, P3 population

| Seq | |
|---|---|
| ID | |
| NO. | Amino acid sequence |
| 1068 | DIQMTQSPSSLSASVGDRVTITCRASQDIYNYLNWYQQKPGKAVKLLIYYTSVLHSGVPSRFS |
| | GSGSGTDYTLTISSLQPEDFATYFCQQGDTLPWTFGQGTKLEIK |
| 1069 | DIQMTQSPSSLSASVGDRVTITCRASQDIYNYLNWYQQKPGKAVKLLIYYTSNLHSGVPSRFS |
| | GSGSGTDYTLTISSLQPEDFATYFCQQGDTLPWTFGQGTKLEIK |
| 1070 | DIQMTQSPSSLSASVGDRVTITCRASQDITNYLNWYQQKPGKAVKLLIYYTSRLHSGVPSRFS |
| | GSGSGTDYTLTISSLQPEDFATYFCQQGDTLPWTFGQGTKLEIK |
| 1071 | DIQMTQSPSSLSASVGDRVTITCGASQDIYNYLNWYQQKPGKAVKLLIYYTSRLHSGVPSRFS |
| | GSGSGTDYTLTISSLQPEDFATYFCQQGDTLPWTFGQGTKLEIK |
| 1072 | DIQMTQSPSSLSASVGDRVTITCRASQDITSYLNWYQQKPGKAVKLLIYYTSNLHSGVPSRFS |
| | GSGSGTDYTLTISSLQPEDFATYFCQQGDTLPWTFGQGTKLEIK |
| 1073 | DIQMTQSPSSLSASVGDRVTITCRASQDIYNYLNWYQQKPGKAVKLLIYYTSRLHSGVPSRFS |
| | GSGSGTDYTLTISSLQPEDFATYFCQQGDTLPWTFGQGTKLEIK |
| 1074 | DIQMTQSPSSLSASVGDRVTITCRASQDIFQYLNWYQQKPGKAVKLLIYYTSKLHSGVPSRFS |
| | GSGSGTDYTLTISSLOPEDFATYFCQQGDTLPWTFGQGTKLEIK |
| 1075 | DIQMTQSPSSLSASVGDRVTITCRASQDIYNYLNWYQQKPGKAVKLLIYYTSALHSGVPSRFS |
| | GSGSGTDYTLTINSLQPEDFATYFCQQGDTLPWTFGQGTKLEIK |
| 1076 | DIQMTQSPSSLSASVGDRVTITCRASQDIYNYLNWYQQKPGKAVKLLIYYTSYLHSGVPSRFS |
| | GSGSGTDYTLTISSLOPEDFATYFCQQGDTLPWTFGQGTKLEIK |
| 1077 | DIQMTQSPSSLSASVGDRVTITCRASQDIYNYLNWYQQKPGKAVKLLIYYTSNLHSGVPSRFS |
| | GSGSGTDYTLTISSLQPEDFATYFCQQGDELPWTFGQGTKLEIK |
| 1078 | DIQMTQSPSSLSASVGDRVTITCRASQDIFNYLNWYQQKPGKAVKLLIYYTSILHSGVPSRFS |
| | GSGSGTDYTLTISSLQPEDFATYFCQQGDTLPWTFGQGTKLEIK |
| 1079 | DIQMTQSPSSLSASVGDRVTITCRASQDIYYYLNWYQQKPGKAVKLLIYYTSELHSGVPSRFS |
| | GSGSGTDYTLTISSLQPEDFATYFCQQGDTLPWTFGQGTKLEIK |
| 1080 | DIQMTQSPSSLSASVGDRVTITCRASQDIYEYLNWYQQKPGKAVKLLIYYTSRLHSGVPSRFS |
| | GSGSGTDYTLTISSLQPEDFATYFCQQGDTLPWTFGQGTKLEIK |
| 1081 | DIQMTQSPSSLSASVGDRVTITCRASQDIYYYLNWYQQKPGKAVKLLIYYTSRLHSGVPSRFS |
| | GSGSGTDYTLTISSLQPEDFATYFCQQGDTLPWTFGQGTKLEIK |
| 1082 | DIQMTQSPSSLSASVGDRVTITCRASQDIYNYLNWYQQKPGKAVKLLIYYTSRLHSGVPSRFS |
| | GSGSGTDYTLTISSLQPEDFATYFCQQGDTPPWTFGQGTKLEIK |
| 1083 | DIQMTQSPSSLSASVGDRVTITCRASQDIYNYLNWYQQKPGKAVKLLIYYTSSLHSGVPSRFS |
| | GSGSGTDYTLTISSLQPEDFATYFCQQGDTLPWTFGQGTKLEIK |
| 1084 | DIQMTQSPSSLSASVGDRVTITCRASQDIYNYLNWYQQKPGKAVKLLIYYTSRLHSGVPSRFS |
| | GSGSGTDYTLTISSLQPEDFATYFCQQGDTQPWTFGQGTKLEIK |
| 1085 | DIQMTQSPSSLSVSVGDRVTITCGASQDIFNYLNWYQQKPGKAVKLLIYYTSRLHSGVPSRFS |
| | GSGSGTDYTLTISSLQPEDFATYFCQQGDGLPWTFGQGTKLEIK |
| 1086 | DIQMTQSPSSLSASVGDRVTITCRASQDIFHYLNWYQQKPGKAVKLLIYYTSILHSGVPSRFS |
| | GSGSGTDYTLTISSLQPEDFATYFCQQGDTLPWTFGQGTKLEIK |
| 1087 | DIQMTQSPSSLSASVGDRVTITCRASQDIYNYLNWYQQKPGKAVKLLIYYTSRLHSGVPSRFS |
| | GSGSGTDYTLTISSLQPEDFATYFCQQGDDLPWTFGQGTKLEIK |
| 1088 | DIQMTQSPSSLSASVGDRVTITCRASQDIYNYLNWYQQKPGKAVKLLIYYTSHLHSGVPSRFS |
| | GSGSGTDYTLTISSLQPEDFATYFCQQGDTLPWTFGQGTKLEIK |

TABLE 27
20E6 VH variants isolated from TRIM library after
2 hour off-rate selection, P4 population

| Seq | |
|---|---|
| ID | |
| NO. | Amino acid sequence |
| 1089 | EVQLVQSGAEVKKPGASVKVSCKASGYTFTSYDMHWVRQAPGQGLEWMGRIDPQGGSVKYAQ |
| | KFQGRATLTVDTSTSTAYMELSRLRSDDTAVYYCARWDHGGNFDVWGQGTLVTVSS |
| 1090 | EVQLVQSGAEVKKPGASVKVSCKASGYTFTSYQVHWVRQAPGQGLEWMGRIDPQSGGIKYAQ |
| | KFQGRATLTVDTSTSTAYMELSRLRSDDTAVYYCARWDYGGYFDVWGQGTLVTVSS |
| 1091 | EVQLVQSGAEVKKPGASVKVSCKASGYTFTDYDMHWVRQAPGQGLEWMGRIDPVSGSIRYAQ |
| | KFQGRATLTVDTSTSTAYMELSRLRSDDTAVYYCARYDHGGYFDVWGQGTLVTVSS |
| 1092 | EVQLVQSGAEVKKPGASVKVSCKASGYTFTSYDMHWVRQAPGQGLEWMGRIDPQSGGIKYAQ |
| | KFQGRATLTVDTSTSTAYMELSRLRSDDTAVYYCARWDYGGYFDVWGQGTLVTVSS |
| 1093 | EVQLVQSGAEVKKPGASVKVSCKASGYTFTSYEIVWVRQAPGQGLEWMGRIDPQSGDIAYAQ |
| | KFQGRATLTVDTSTSTAYMELSRLRSDDTAVYYCARWDHGGYFDVWGQGTLVTVSS |
| 1094 | EVQLVQSGAEVKKPGASVKVSCKASGYTFTRYDLHWVRQAPGQGLEWMGWIDPKSGSLKYAQ |
| | KFQGRATLTVDTSTSTAYMELSRLRSDDTAVYYCARWDYGGYFDVWGQGTLVTVSS |
| 1095 | EVQLVQSGAEVKKPGASVKVSCKASGYTFTTYWMHWVRQAPGQGLEWMGRIDPQRGDIKYAQ |
| | KFQGRATLTVDTSTSTAYMELSRLRSDDTAVYYCARWDYGGFFDVWGQGTLVTVSS |
| 1096 | EVQLVQSGAEVKKPGASVKVSCKASGYTFTSYWLHWVRQAPGQGLEWMGRIDPQSGGIIYAQ |
| | KFQGRATLTVDTSTSTAYMELSRLRSDDTAVYYCARWDYGGYFDVWGQGTLVTVSS |
| 1097 | EVQLVQSGAEVKKPGASVKVSCKASGYTFTDYDMHWVROAPGQGLEWMGRIDPQSGDHDYAQ |
| | KFQGRATLTVDTSTSTAYMELSRLRSDDTAVYYCARYDHGGYFDVWGQGTLVTVSS |
| 1098 | EVQLVQSGAEVKKPGASVKVSCKASGYTFTSYDMHWVRQAPGQGLEWMGRIDPQSGGHKYAQ |
| | KFQGRATLTVDTSTSTAYMELSRLRSDDTAVYYCARYDHGGYFDVWGQGTLVTVSS |
| 1099 | EVQLVQSGAEVKKPGASVKVSCKASGYTFTSYDMQWVRQAPGQGLEWMGRIDPISGSIKYAQ |
| | KFQGRATLTVDTSTSTAYMELSRLRSDDTAVYYCARYDHGGYFDVWGQGTLVTVSS |
| 1100 | EVQLVQSGAEVKKPGASVKVSCKASGYTFTSYEMQWVRQAPGQGLEWMGRIDPQSGSIKYAQ |
| | KFQGRATLTVDTSTSTAYMELSRLRSDDTAVYYCARWDYGGYFDVWGQGTLVTVSS |
| 1101 | EVQLVQSGAEVKKPGASVKVSCKASGYTFTGYDLHWVRQAPGQGLEWMGRIDPQSGDDRYAQ |
| | KFQGRATLTVDTSTSTAYMELSRLRSDDTAVYYCARWDEGGLFDVWGQGTLVTVSS |
| 1102 | EVQLVQSGAEVKKPGASVKVSCKASGYTFTSYDMHWVRQAPGQGLEWMGRIDPYGGGIKYAQ |
| | KFQGRATLTVDTSTSTAYMELSRLRSDDTAVYYCARYDHGGYFDVWGQGTLVTVSS |
| 1103 | EVQLVQSGAEVKKPGASVKVSCKASGYTFTSYDMHWVRQAPGQGLEWMGRIDPQGGNYYAQK |
| | FQGRATLTVDTSTSTAYMELSRLRSDDTAVYYCARWDYGGYFDVWGQGTLVTVSS |
| 1104 | EVQLVQSGAEVKKPGASVKVSCKASGYTFTSYDMHWVRQAPGQGLEWMGRIDPDSGGIKYAQ |
| | KFQGRATLTVDTSTSTAYMELSRLRSDDTAVYYCARWDYSGYFDVWGQGTLVTVSS |
| 1105 | EVQLVQSGAEVKKPGASVKVSCKASGYTFTSYFMSWVRQAPGQGLEWMGRIDPQSGGIKYAQ |
| | KFQGRATLTVDTSTSTAYMELSRLRSDDTAVYYCARWDYAGYFDVWGQGTLVTVSS |
| 1106 | EVQLVQSGAEVKKPGASVKVSCKASGYTFTSYDMHWVRQAPGQGLEWMGRIDPQSGGIKYAQ |
| | KFQGRATLTVDTSTSTAYMELSRLRSDDTAVYYCARYDHGGYFDVWGQGTLVTVSS |
| 1107 | EVQLVQSGAEVKKPGASVKVSCKASGYTFTAYDLHWVRQAPGQGLEWMGRIDPQSGGIKYAQ |
| | KFQGRATLTVDTSTSTAYMELSRLRSDDTAVYYCARWDQGGYFDVWAQGTLVTVSS |
| 1108 | EVQLVQSGAEVKKPGASVKVSCKASGYTFTPYQMHWVRQAPGQGLEWMGRIDPQSGGIPYAQ |
| | KFQGRATLTVDTSTSTAYMELSRLRSDDTAVYYCARWDYGGYFDVWGQGTLVTVSS |
| 1109 | EAQLVQSGAEVKKPGASVKVSCKASGYTFTSYWMHWVRQAPGQGLEWMGRIDPQSGGIKYAQ |
| | KFQGRATLTVDTSTSTAYMELSRLRSDDTAVYYCARWDYGGYFDVWGQGTLVTVSS |
| 1110 | EVQLVQSGAEVKKPGASVKVSCKASGYTFTSYDLHWVRQAPGQGLEWMGRIDPQSGGIKYAQ |
| | KFQGRATLTVDTSTSTAYMELSRLRSDDTAVYYCARYDQGGYFDVWGQGTLVTVSS |
| 1111 | EVQLVQSGAEVKKPGASVKVSCKASGYTFTSYDMHWVRQAPGQGLEWMGRIDPQSGGIIYAQ |
| | KFQGRATLTVDTSTSTAYMELSRLRSDDTAVYYCARYDHGGYFDVWGQGTLVTVSS |
| 1112 | EVQLVQSGAEVKKPGASVKVSCKASGYTFTNYWMHWVRQAPGQGLEWMGRIDPQSGGIKYAQ |
| | KFQGRATLTVDTSTSTAYMELSRLRSDDTAVYYCARWDYGGYFDVWGQGTLVTVSS |
| 1113 | EVQLVQSGAEVKKPGASVKVSCKASGYTFTHYDMHWVRQAPGQGLEWMGRIDPQSGGHKYAQ |
| | KFQGRATLTVDTSTSTAYMELSRLRSDDTAVYYCARWDHGGYFDVWGQGTLVTVSS |

TABLE 28
20E6 VL variants isolated from TRIM library after
2 hour off-rate selection, P4 population

| Seq | |
|---|---|
| ID | Amino acid sequence |
| 1114 | DIQMTQSPSSLSASVGDRVTITCRASQDIYSYLNWYQQKPGKAVKLLIYYTSNLHSGVPSRFS |
| | GSGSGTDYTLTISSLQPEDFATYFCQQGDTLPWTFGQGTKLEIK |
| 1115 | DIQMTQSPSSLSASVGDRVTITCRASQDIFNYLNWYQQKPGKAVKLLIYYTSRLHSGVPSRFS |
| | GSGSGTDYTLTISSLQPEDFATYFCQQGDTLPWTFGQGTKLEIK |
| 1116 | DIQMTQSPSSLSASVGDRVTITCRASQDITNYLNWYQQKPGKAVKLLIYYTSRLHSGVPSRFS |
| | GSGSGTDYTLTISSLQPEDFATYFCQQGDTLPWTFGQGTKLEIK |
| 1117 | DIQMTQSPSSLSASVGDRVTITCRASQDIFSYLNWYQQKPGKAVKLLIYYTSILHSGVPSRFS |
| | GSGSGTDYTLTISSLQPEDFATYFCQQGDTLPWTFGQGTKLEIK |
| 1118 | DIQMTQSPSSLSASVGDRVTITCRASQDITNYLNWYQQKPGKAVKLLIYYTSQLHSGVPSRFS |
| | GSGSGTDYTLTISSLQPEDFATYFCQQGDELPWTFGQGTKLEIK |
| 1119 | DIQMTQSPSSLSASVGDRVTITCRASQDIYNYLNWYQQKPGKAVKLLIYYTSYLHSGVPSRFS |
| | GSGSGTDYTLTISSLQPEDFATYFCQQGDTQPWTFGQGTKLEIK |
| 1120 | DIQMTQSPSSLSASVGDRVTITCRASQDIYSYLNWYQQKPGKAVKLLIYYTSQLHSGVPSRFS |
| | GSGSGTDYTLTISSLQPEDFATYFCQQGDTLPWTFGQGTKLEIK |
| 1121 | DIQMTQSPSSLSASVGDRVTITCRASQDIYNYLNWYQQKPGKAVKLLIYYTSRLHSGVPSRFS |
| | GSGSGTDYTLTISSLQPEDFATYFCQQGDTQPWTFGQGTKLEIK |
| 1122 | DIQMTQSPSSLSASVGDRVTITCRASQDIYHYLNWYQQKPGKAVKLLIYYTSRLHSGVPSRFS |
| | GSGSGTDYTLTISSLQPEDFATYFCQQGDGLPWTFGQGTKLEIK |
| 1123 | DIQMTQSPSSLSASVGDRVTITCRASQDIYSYLNWYQQKPGKAVKLLIYYTSGLHSGVPSRFS |
| | GSGSGTDYTLTISSLOPEDFATYFCQQGDTLPWTFGQGTKLEIK |
| 1124 | DIQMTQSPSSLSASVGDRVTITCRASQDIYNYLNWYQQKPGKAVKLLIYYTSVLHSGVPSRFS |
| | GSGSGTDYTLTISSLQPEDFATYFCQQGDTLPWTFGQGTKLEIK |
| 1125 | DIQMTQSPSSLSASVGDRVTITCRASQDIFYYLNWYQQKPGKAVKLLIYYTSRLHSGVPSRFS |
| | GSGSGTDYTLTISSLQPEDFATYFCQQGDTLPWTFGQGTKLEIK |
| 1126 | DIQMTQSPSSLSASVGDRVTITCRASQDIYNYLNWYQQKPGKAVKLLIYYTSRLHSGVPSRFS |
| | GSGSGTDYTLTISSLQPEDFATYFCQQGDALPWTFGQGTKLEIK |
| 1127 | DIQMTQSPSSLSASVGDRVTITCRASQDIYNYLNWYQQKPGKAVKLLIYYTSHLHSGVPSRFS |
| | GSGSGTDYTLTISSLQPEDFATYFCQQGDTLPWTFGQGTKLEIK |
| 1128 | DIQMTQSPSSLSASVGDRVTITCRASQDITNYLNWYQQKPGKAVKLLIYYTSELHSGVPSRFS |
| | GSGSGTDYTLTISSLQPEDFATYFCQQGDTLPWTFGQGTKLEIK |
| 1129 | DIQMTQSPSSLSASVGDRVTITCRASQDIYNYLNWYQQKPGKAVKLLIYYTSRLHSGVPSRFS |
| | GSGSGTDYTLTISSLQPEDFATYFCQQGDTLPWTFGQGTKLEIK |
| 1130 | DIQMTQSPSSLSASVGDRVTITCRASQDITKYLNWYQQKPGKAVKLLIYYTSRLHSGVPSRFS |
| | GSGSGTDYTLTISSLQPEDFATYFCQQGDTLPWTFGQGTKLEIK |
| 1131 | DIQMTQSPSSLSASVGDRVTITCRASQDIYKYLNWYQQKPGKAVKLLIYYTSELHSGVPSRFS |
| | GSGSGTDYTLTISSLOPEDFATYFCQQGDTLPWTFGQGTKLEIK |
| 1132 | DIQMTQSPSSLSASVGDRVTITCRASQDIFSYLNWYQQKPGKAVKLLIYYTSRLHSGVPSRFS |
| | GSGSGTDYTLTISSLQPEDFATYFCQQGDTLPWTFGQGTKLEIK |
| 1133 | DIQMTQSPSSLSASVGDRVTITCRASQDIYHYLNWYQQKPGKAVKLLIYYTSRLHSGVPSRFS |
| | GSGSGTDYTLTISSLQPEDFATYFCQQGDTLPWTFGQGTKLEIK |
| 1134 | DIQMTQSPSSLSASVGDRVTITCRASQDIYNYLNWYQQKPGKAVKLLIYYTSALHSGVPSRFS |
| | GSGSGTDYTLTISSLQPEDFATYFCQQGDTLPWTFGQGTKLEIK |
| 1135 | DIQMTQSPSSLSASVGDRVTITCRASQDIYYYLNWYQQKPGKAVKLLIYYTSRLHSGVPSRFS |
| | GSGSGTDYTLTISSLQPEDFATYFCQQGDTLPWTFGQGTKLEIK |
| 1136 | DIQMTQSPSSLSASVGDRVTITCRASQDIYNYLNWYQQKPGKAVKLLIYYTSRLHSGVPSRFS |
| | GSGSGTDYTLTISSLQPEDFATYFCQQGDTVPWTFGQGTKLEIK |
| 1137 | DIQMTQSPSSLSASVGDRVTITCRASQDIYNYLNWYQQKPGKAVKLLIYYQSALHSGVPSRFS |
| | GSGSGTDYTLTISSLQPEDFATYFCQQGDTLPWTFGQGTKLEIK |

TABLE 29
20E6 VH variants isolated from TRIM library
after 12 hour off-rate selection

| Seq ID NO. | Amino acid sequence |
|---|---|
| 1138 | EVQLVQSGAEVKKPGASVKVSCKASGYTFTHYDMHWVRQAPGQGLEWMGRIDPQGGGIKYAQK |
| | FQGRATLTVDTSTSTAYMELSRLRSDDTAVYYCARWDYGGQFDVWGQGTLVTVSS |
| 1139 | EVQLVQSGAEVKKPGASVKVSCKASGYTFTSYLMQWVRQAPGQGLEWMGRIDPQDGDIKYAQK |
| | FQGRATLTVDTSTSTAYMELSRLRSDDTAVYYCARWDHGGYFDVWGQGTLVTVSS |
| 1140 | EVQLVQSGAEVKKPGASVKVSCKASGYTFTSYDMHWVRQAPGQGLEWMGRIDPQDGGIKYAQK |
| | FQGRATLTVDTSTSTAYMELSRLRSDDTAVYYCARWDYGGYFDVWGQGTLVTVSS |
| 1141 | EVQLVQSGAEVKKPGASVKVSCKASGYTFTSYYMNWVRQAPGQGLEWMGRIDPDDGDIKYAQK |
| | FQGRATLTVDTSTSTAYMELSRLRSDDTAVYYCARWDYGGYFDVWGQGTLVTVSS |
| 1142 | EVQLVQSGAEVKKPGASVKVSCKASGYTFTSYDMHWVRQAPGQGLEWMGRIDPQSGGIRYAQK |
| | FQGRATLTVDTSTSTAYMELSRLRSDDTAVYYCARWDYGGYFDVWGQGTLVTVSS |
| 1143 | EVQLVQSGAEVKKPGASVKVSCKASGYTFTKYLMSWVRQAPGQGLEWMGRIDPQDGDIKYAQK |
| | FQGRATLTVDTSTSTAYMELSRLRSDDTAVYYCARWDYGGYFDVWGQGTLVTVSS |
| 1144 | EVQLVQSGAEVKKPGASVKVSCKASGYTFTSYDMHWVRQAPGQGLEWMGRIDPQDGGSKYAQK |
| | FQGRATLTVDTSTSTAYMELSRLRSDDTAVYYCARWDYGGYFDVWGQGTLVTVSS |
| 1145 | EVQLVQSGAEVKKPGASVKVSCKASGYTFTSYYINWVRQAPGQGLEWMGRIDPQSGGIKYAQK |
| | FQGRATLTVDTSTSTAYMELSRLRSDDTAVYYCARYDHGGYFDVWGQGTLVTVSS |
| 1146 | EVQLVQSGAEVKKPGASVKVSCKASGYTFTSYDLHWVRQAPGQGLEWMGRIDPYDGGTKYAQK |
| | FQGRATLTVDTSTSTAYMELSRLRSDDTAVYYCARWDYGGYFDVWGQGTLVTVSS |
| 1147 | EVQLVQSGAEVKKPGASVKVSCKASGYTFTNYDMHWVRQAPGQGLEWMGRIDPQGGGIRYAQK |
| | FQGRATLTVDTSTSTAYMELSRLRSDDTAVYYCARWDYGGYFDVWGQGTLVTVSS |
| 1148 | EVQLVQSGAEVKKPGASVKVSCKASGYTFTSYDMHWVRQAPGQGLEWMGRIDPPSGGIKYAQK |
| | FQGRATLTVDTSTSTAYMELSRLRSDDTAVYYCARWHTGGYFDVWGQGTLVTVSS |
| 1149 | EVQLVQSGAEVKKPGASVKVSCKASGYTFTSYDMHWVRQAPGQGLEWMGRIDPDGGGIKYAQK |
| | FQGRATLTVDTSTSTAYMELSRLRSDDTAVYYCARWDYGGQFDVWGQGTLVTVSS |
| 1150 | EVQLVQSGAEVKKPGASVKVSCKASGYTFTSYLMQWVRQAPGQGLEWMGRIDPSDGGTKYAQK |
| | FQGRATLTVDTSTSTAYMELSRLRSDDTAVYYCARWDYGGYFDVWGQGTLVTVSS |
| 1151 | EVQLVQSGAEVKKPGASVKVSCKASGYTFTSYLMQWVRQAPGQGLEWMGRIDPIDGGIKYAQK |
| | FQGRATLTVDTSTSTAYMELSRLRSDDTAVYYCARWHYGGYFDVWGQGTLVTVSS |
| 1152 | EVQLVQSGAEVKKPGASVKVSCKASGYTFTSYYMNWVRQAPGQGLEWMGRIDPFDGSIKYAQK |
| | FQGRATLTVDTSTSTAYMELSRLRSDDTAVYYCARWDYGGYFDVWGQGTLVTVSS |
| 1153 | EVQLVQSGAEVKKPGASVKVSCKASGYTFTYYDMHWVRQAPGQGLEWMGRIDPQSGGIKYAQK |
| | FQGRATLTVDTSTSTAYMELSRLRSDDTAVYYCARWDYGGQFDVWGQGTLVTVSS |
| 1154 | EVQLVQSGAEVKKPGASVKVSCKASGYTFTHYDMHWVRQAPGQGLEWMGRIDPVSGGIKYAQK |
| | FQGRATLTVDTSTSTAYMELSRLRSDDTAVYYCARWDYGGQFDVWGQGTLVTVSS |
| 1155 | EVQLVQSGAEVKKPGASVKVSCKASGYTFTSYDIHWVRQAPGQGLEWMGRIDPISGGIKYAQK |
| | FQGRATLTVDTSTSTAYMELSRLRSDDTAVYYCARWDYGGYFDVWGQGTLVTVSS |
| 1156 | EVQLVQSGAEVKKPGASVKVSCKASGYTFTSYYMNWVRQAPGQGLEWMGRIDPQSGGVKYAQK |
| | FQGRATLTVDTSTSTAYMELSRLRSDDTAVYYCARWHYGGQFDVWGQGTLVTVSS |
| 1157 | EVQLVQSGAEVKKPGASVKVSCKASGYTETSYDMHWVRQAPGQGLEWMGRIDPQSGGQKYAQK |
| | FQGRATLTVDTSTSTAYMELSRLRSDDTAVYYCARWDYGGQFDVWGQGTLVTVSS |
| 1158 | EVQLVQSGAEVKKPGASVKVSCKASGYTFTRYDMHWVRQAPGQGLEWMGRIDPQSGGTKYAQK |
| | FQGRATLTVDTSTSTAYMELSRLRSDDTAVYYCARWHTGGQFDVWGQGTLVTVSS |
| 1159 | EVQLVQSGAEVKKPGASVKVSCKASGYTETHYDMHWVRQAPGQGLEWMGRIDPQTGGIKYAQK |
| | FQGRATLTVDTSTSTAYMELSRLRSDDTAVYYCARWDYGGQFDVWGQGTLVTVSS |
| 1160 | EVQLVQSGAEVKKPGASVKVSCKASGYTFTQYDLHWVRQAPGQGLEWMGRIDPQTGGIKYAQK |
| | FQGRATLTVDTSTSTAYMELSRLRSDDTAVYYCARWDYGGQFDVWGQGTLVTVSS |
| 1161 | EVQLVQSGAEVKKPGASVKVSCKASGYTFTSYDLHWVRQAPGQGLEWMGRIDPYSGGIKYAQK |
| | FQGRATLTVDTSTSTAYMELSRLRSDDTAVYYCARWDYGGYFDVWGQGTLVTVSS |
| 1162 | EVQLVQSGAEVKKPGASVKVSCKASGYTFTSYDMHWVRQAPGQGLEWMGRIDPISGGIKYAQK |
| | FQGRATLTVDTSTSTAYMELSRLRSDDTAVYYCARWDYGGYFDVWGQGTLVTVSS |
| 1163 | EVQLVQSGAEVKKPGASVKVSCKASGYTFTSYDKHWVRQAPGQGLEWMGRIDPASGGIRYAQK |
| | FQGRATLTVDTSTSTAYMELSRLRSDDTAVYYCARWDYGGQFDVWGQGTLVTVSS |
| 1164 | EVQLVQSGAEVKKPGASVKVSCKASGYTFTSYDTHWVRQAPGQGLEWMGRIDPQDGGVKYAQK |
| | FQGRATLTVDTSTSTAYMELSRLRSDDTAVYYCARWDYGGYFDVWGQGTLVTVSS |
| 1165 | EVQLVQSGAEVKKPGASVKVSCKASGYTFTSYDNHWVRQAPGQGLEWMGRIDPQSGGVIYAQK |
| | FQGRATLTVDTSTSTAYMELSRLRSDDTAVYYCARWDYGGYFDVWGQGTLVTVSS |
| 1166 | EVQLVQSGAEVKKPGASVKVSCKASGYTFTQYDIHWVRQAPGQGLEWMGRIDPTSGGIKYAQK |
| | FQGRATLTVDTSTSTAYMELSRLRSDDTAVYYCARWDYGGYFDVWGQGTLVTVSS |
| 1167 | EVQLVQSGAEVKKPGASVKVSCKASGYTFTSYDLHWVRQAPGQGLEWMGRIDPQSGGIRYAQK |
| | FQGRATLTVDTSTSTAYMELSRLRSDDTAVYYCARWDYGGQFDVWGQGTLVTVSS |
| 1168 | EVQLVQSGAEVKKPGASVKVSCKASGYTFTSYDLHWVRQAPGQGLEWMGRIDPPTGGIKYAQK |
| | FQGRATLTVDTSTSTAYMELSRLRSDDTAVYYCARWDYGGYFDVWGQGTLVTVSS |
| 1169 | EVQLVQSGAEVKKPGASVKVSCKASGYTFTEYDMHWVRQAPGQGLEWMGRIDPQSGGIRYAQK |
| | FQGRATLTVDTSTSTAYMELSRLRSDDTAVYYCARWDYGGQFDAWGQGTLVTVSS |
| 1170 | EVQLVQSGAEVKKPGASVKVSCKASGYTFTLYDMHWVRQAPGQGLEWMGRIDPVSGGIKYAQK |
| | FQGRATLTVDTSTSTAYMELSRLRSDDTAVYYCARWDYGGQFDVWGQGTLVTVSS |
| 1171 | EVQLVQSGAEVKKPGASVKVSCKASGYTFTSYDMHWVRQAPGQGLEWMGRIDPQSGGTKYAQK |
| | FQGRATLTVDTSTSTAYMELSRLRSDDTAVYYCARWDYGGQFDVWGQGTLVTVSS |
| 1172 | EVQLVQSGAEVKKPGASVKVSCKASGYTFTSYDMHWVRQAPGQGLEWMGRIDPQSGGIKYAQK |
| | FQGRATLTVDTSTSTAYMELSRLRSDDTAVYYCARWDYGGQFDVWGQGTLVTVSS |
| 1173 | EVQLVQSGAEVKKPGASVKVSCKASGYTFTSYDIHWVRQAPGQGLEWMGRIDPVSGGTKYAQK |
| | FQGRATLTVDTSTSTAYMELSRLRSDDTAVYYCARWDYGGQFDVWGQGTLVTVSS |
| 1174 | EVQLVQSGAEVKKPGASVKVSCKASGYTFTSYDLHWVRQAPGQGLEWMGRIDPQSGGIKYAQK |
| | FQGRATLTVDTSTSTAYMELSRLRSDDTAVYYCARWDYGGQFDVWGQGTLVTVSS |
| 1175 | EVQLVQSGAEVKKPGASVKVSCKASGYTFTSYFLNWVRQAPGQGLEWMGRIDPQIGDSKYAQK |
| | FQGRATLTVDTSTSTAYMELSRLRSDDTAVYYCARWDYGGYFDVWGQGTLVTVSS |
| 1176 | EVQLVQSGAEVKKPGASVKVSCKASGYTFTSYDVHWVRQAPGQGLEWMGRIDPQSGGARYAQK |
| | FQGRATLTVDTSTSTAYMELSRLRSDDTAVYYCARWDYGGQFDVWGQGTLVTVSS |
| 1177 | EVQLVQSGAEVKKPGASVKVSCKASGYTFTSYDMHWVRQAPGQGLEWMGRIDPQSGGIRYAQK |
| | FQGRATLTVDTSTSTAYMELSRLRSDDTAVYYCARWDYGGQFDVWGQGTLVTVSS |
| 1178 | EVQLVQSGAEVKKPGASVKVSCKASGYTFTVYEMQWVRQAPGQGLEWMGRIDPQSGSNHYAQK |
| | FQGRATLTVDTSTSTAYMELSRLRSDDTAVYYCARWDYGGYFDVWGQGTLVTVSS |
| 1179 | EVQLVQSGAEVKKPGASVKVSCKASGYTFTSYYFHWVRQAPGQGLEWMGRIDPQDGDIKYAQK |
| | FQGRATLTVDTSTSTAYMELSRLRSDDTAVYYCARWDYGGYFDVWGQGTLVTVSS |

TABLE 30
20E6 VL variants isolated from TRIM library
after 12 hour off-rate selection

| Seq ID NO. | Amino acid sequence |
|---|---|
| 1180 | DIQMTQSPSSLSASVGDRVTITCRASQDIYNYLNWYQQKPGKAVKLLIYYTSELHSGVPSRF |
| | SGSGSGTDYTLTISSLQPEDFATYFCQQGDVLPWTFGQGTKLEIK |
| 1181 | DIQMTQSPSSLSASVGDRVTITCRASQDIYNYLNWYQQKPGKAVKLLIYYTSILHSGVPSRF |
| | SGSGSGTDYTLTISSLQPEDFATYFCQQGDALPWTFGQGTKLEIK |
| 1182 | DIQMTQSPSSLSASVGDRVTITCRASQDIYNYLNWYQQKPGKAVKLLIYYTSRLHSGVPSRF |
| | SGSGSGTDYTLTISSLQPEDFATYFCQQGDELPWTFGQGTKLEIK |
| 1183 | DIQMTQSPSSLSASVGDRVTITCRASQDIYSYLNWYQQKPGKAVKLLIYYTSNLHSGVPSRF |
| | SGSGSGTDYTLTISSLQPEDFATYFCQQGDTLPWTFGQGTKLEIK |
| 1184 | DIQMTQSPSSLSASVGDRVTITCRASQDIYSYLNWYQQKPGKAVKLLIYYTSILHSGVPSRF |
| | SGSGSGTDYTLTISSLQPEDFATYFCQQGDTLPWTFGQGTKLEIK |
| 1185 | DIQMTQSPSSLSASVGDRVTITCRASQDIYNYLNWYQQKPGKAVKLLIYYTSRLHSGVPSRF |
| | SGSGSGTDYTLTISSLQPEDFATYFCQQGDNLPWTFGQGTKLEIK |
| 1186 | DIQMTQSPSSLSASVGDRVTITCRASQDIYNYLNWYQQKPGKAVKLLIYYTSGLHSGVPSRF |
| | SGSGSGTDYTLTISSLQPEDFATYFCQQGDALPWTFGQGTKLEIK |
| 1187 | DIQMTQSPSSLSASVGDRVTITCRASQDIYNYLNWYQQKPGKAVKLLIYYTSVLHSGVPSRF |
| | SGSGSGTDYTLTISSLQPEDFATYFCQQGDTLPWTFGQGTKLEIK |
| 1188 | DIQMTQSPSSLSASVGDRVTITCRASQDIYHYLNWYQQKPGKAVKLLIYYTSHLHSGVPSRF |
| | SGSGSGTDYTLTISSLQPEDFATYFCQQGDEIPWTFGQGTKLEIK |
| 1189 | DIQMTQSPSSLSASVGDRVTITCRASQDIYNYLNWYQQKPGKAVKLLIYYTSHLHSGVPSRF |
| | SGSGSGTDYTLTISSLQPEDFATYFCQQGDTLPWTFGQGTKLEIK |
| 1190 | DIQMTQSPSSLSASVGDRVTITCRASQDIYNYLNWYQQKPGKAVKLLIYYTSPLHSGVPSRF |
| | SGSGSGTDYTLTISSLQPEDFATYFCQQGDTLPWTFGQGTKLEIK |
| 1191 | DIQMTQSPSSLSASVGDRVTITCRASQDIYKYLNWYQQKPGKAVKLLIYYTSILHSGVPSRF |
| | SGSGSGTDYTLTISSLQPEDFATYFCQQGDELPWTFGQGTKLEIK |
| 1192 | DIQMTQSPSSLSASVGDRVTITCRASQDIYKYLNWYQQKPGKAVKLLIYYTSNLHSGVPSRF |
| | SGSGSGTDYTLTISSLQPEDFATYFCQQGDEIPWTFGQGTKLEIK |
| 1193 | DIQMTQSPSSLSASVGDRVTITCRASQDIWNYLNWYQQKPGKAVKLLIYYTSRLHSGVPSRF |
| | SGSGSGTDYTLTISSLQPEDFATYFCQQGDTLPWTFGQGTKLEIK |
| 1194 | DIQMTQSPSSLSASVGDRVTITCRASQDIFYYLNWYQQKPGKAVKLLIYYTSVLHSGVPSRF |
| | SGSGSGTDYTLTISSLQPEDFATYFCQQGDTLPWTFGQGTKLEIK |
| 1195 | DIQMTQSPSSLSASVGDRVTITCRASQDIYNYLNWYQQKPGKAVKLLIYYTSVLHSGVPSRF |
| | SGSGSGTDYTLTISSLQPEDFATYFCQQGDELPWTFGQGTKLEIK |
| 1196 | DIQMTQSPSSLSASVGDRVTITCRASQDIFNYLNWYQQKPGKAVKLLIYYTSILHSGVPSRF |
| | SGSGSGTDYTLTISSLQPEDFATYFCQQGDTLPWTFGQGTKLEIK |
| 1197 | DIQMTQSPSSLSASVGDRVTITCRASQDIWKYLNWYQQKPGKAVKLLIYYTSILHSGVPSRF |
| | SGSGSGTDYTLTISSLQPEDFATYFCQQGDTIPWTFGQGTKLEIK |
| 1198 | DIQMTQSPSSLSASVGDRVTITCRASQDIFNYLNWYQQKPGKAVKLLIYYTSHLHSGVPSRF |
| | SGSGSGTDYTLTISSLQPEDFATYFCQQGDTLPWTFGQGTKLEIK |
| 1199 | DIQMTQSPSSLSASVGDRVTITCRASQDIWNYLNWYQQKPGKAVKLLIYYTSTLHSGVPSRF |
| | SGSGSGTDYTLTISSLQPEDFATYFCQQGDLLPWTFGQGTKLEIK |
| 1200 | DIQMTQSPSSLSASVGDRVTITCRASQDIFNYLNWYQQKPGKAVKLLIYYTSYLHSGVPSRF |
| | SGSGSGTDYTLTISSLQPEDFATYFCQQGDTLPWTFGQGTKLEIK |
| 1201 | DIQMTQSPSSLSASVGDRVTITCRASQDIYSYLNWYQQKPGKAVKLLIYYTSRLHSGVPSRF |
| | SGSGSGTDYTLTISSLQPEDFATYFCQQGDTLPWTFGQGTKLEIK |
| 1202 | DIQMTQSPSSLSASVGDRVTITCRASQDIYNYLNWYQQKPGKAVKLLIYYTSILHSGVPSRF |
| | SGSGSGTDYTLTISSLQPEDFATYFCQQGDELPWTFGQGTKLEIK |
| 1203 | DIQMTQSPSSLSASVGDRVTITCRASQDIYNYLNWYQQKPGKAVKLLIYYTSELHSGVPSRF |
| | SGSGSGTDYTLTISSLQPEDFATYFCQQGDTLPWTFGQGTKLEIK |
| 1204 | DIQMTQSPSSLSASVGDRVTITCRASQDIYNYLNWYQQKPGKAVKLLIYYTSRLHSGVPSRF |
| | SGSGSGTDYTLTISSLQPEDFATYFCQQGDTLPWTFGQGTKLEIK |
| 1205 | DIQMTQSPSSLSASVGDRVTITCRASQDIYNYLNWYQQKPGKAVKLLIYYTSILHSGVPSRF |
| | SGSGSGTDYTLTISSLQPEDFATYFCQQGDTLPWTFGQGTKLEIK |
| 1206 | DIQMTQSPSSLSASVGDRVTITCRASQDIYNYLNWYQQKPGKAVKLLIYYTSGLHSGVPSRF |
| | SGSGSGTDYTLTISSLQPEDFATYFCQQGDTLPWTFGQGTKLEIK |
| 1207 | DIQMTQSPSSLSASVGDRVTITCRASQDIYNYLNWYQQKPGKAVKLLIYYTSHLHSGVPSRF |
| | SGSGSGTDYTLTISSLQPEDFATYFCQQGDALPWTFGQGTKLEIK |
| 1208 | DIQMTQSPSSLSASVGDRVTITCRASQDIYNYLNWYQQKPGKAVKLLIYYTSALHSGVPSRE |
| | SGSGSGTDYTLTISSLQPEDFATYFCQQGDTLPWTFGQGTKLEIK |
| 1209 | DIQMTQSPSSLSASVGDRVTITCRASQDIYNYLNWYQQKPGKAVKLLIYYTSNLHSGVPSRF |
| | SGSGSGTDYTLTISSLQPEDFATYFCQQGDTLPWTFGQGTKLEIK |
| 1210 | DIQMTQSPSSLSASVGDRVTITCRASQDIYNYLNWYQQKPGKAVKLLIYYESRLHSGVPSRE |
| | SGSGSGTDYTLTISSLQPEDFATYFCQQGDELPWTFGQGTKLEIK |
| 1211 | DIQMTQSPSSLSASVGDRVTITCRASQDIYNYLNWYQQKPGKAVKLLIYYTSNLHSGVPSRF |
| | SGSGSGTDYTLTISSLQPEDFATYFCQQGDTMPWTFGQGTKLEIK |

TABLE 31
Consensus of 20E6 variants isolated from Doping Library after 2 hour off-rate
selection, P1 population

| Seq ID NO. | Identifier | Amino acid sequence |
|---|---|---|
| 1212 | VH consensus | EVQLVQSGAEVKKPGASVKVSCKASGYTFTSYYMQWVRQAP |
| | | GQGLEWMGRIDPQSGGIKYAQKFQGRATLTVDTSTSTAYME |
| | | LSRLRSDDTAVYYCARWDYGGYFDVWGQGTLVTVSS |
| 1213 | HCDR1_consensus | SYYMQ |
| | | R WIN |
| | | N LLH |
| | | T D S |
| | | F A |
| 1214 | HCDR2_consensus | RIDPQSGGIKYAQKFQG |
| | | ED DVR |
| | | LR SI |
| | | WN FL |
| | | DI |
| | | G |
| 1215 | HCDR3_consensus | WDYGGYFDV |
| | | W Q |
| 1216 | VL consensus | DIQMTQSPSSLSASVGDRVTITCRASQDIWNYLNWYQQKPG |
| | | KAVKLLIYYTSRLETGVPSRFSGSGSGTDYTLTISSLQPED |
| | | FATYFCQQGDTLPWTFGQGTKLEIK |
| 1217 | LCDR1_consensus | RASQDIWNYLN |
| | | YS |
| | | H |
| | | Y |
| 1218 | LCDR2_consensus | YTSRLET |
| | | S G HS |
| | | M |
| | | V |
| | | S |
| | | L |
| | | W |
| | | I |
| 1219 | LCDR3_consensus | QQGDTLPWT |
| | | AI |
| | | I |
| | | M |

Please note that for the consensus sequences in Table 31 and subsequent tables
that the blank spots below an amino acid residue indicate that the amino acid
residue at that position does not change and is not substituted. In Table 31
and subsequent tables if there is any amino acid below an amino acid residue
then that amino acid residue at that position may be substituted with the
any amino acid listed. For example, for HCDR1 of SEQ ID NO: 1213, the serine
(S) at position 1 may be substituted with any of arginine (R), asparagine (N),
or threonine (T). For SEQ ID NO: 1213, the tyrosine (Y) at position 2 is not
substituted, however the tyrosine (Y) at position 3 may be substituted with
any of tryptophan (W), leucine (L), aspartic acid (D), or phenylalanine (F).
For SEQ ID NO: 1213, the methionine (M) at position 4 may be substituted with
any of: isoleucine (I) or leucine (L). For SEQ ID NO: 1213, the glutamine (Q)
at position 5 may be substituted with any of: asparagine (N), histidine
(H), serine (S), or alanine (A). See below for a representation (with
subcolumns shown) of the description in this paragraph.

| | | |
|---|---|---|
| 1213 | HCDR1_consensus | S Y Y M Q |
| | | R W I N |
| | | N L L H |
| | | T D S |
| | | F A |

TABLE 32
Consensus of 20E6 variants isolated from Doping Library after 2 hour
off-rate selection, P2 population

| Seq ID NO. | Identifier | Amino acid sequence |
|---|---|---|
| 1220 | VH consensus | EVQLVQSGAEVKKPGASVKVSCKASGYTFTSYDMQWVRQAP |
| | | GQGLEWMGRIDPQSGGIKYAQKFQGRATLTVDTSTSTAYME |
| | | LSRLRSDDTAVYYCARWDYGGYFDVWGQGTLVTVSS |
| 1221 | HCDR1_consensus | SYDMQ |
| | | R FIH |
| | | A LLN |
| | | WVY |
| | | Y |
| 1222 | HCDR2_consensus | RIDPQSGGIKYAQKFQG |
| | | ED DVI |
| | | PG TL |
| | | D S |
| | | K |
| 1223 | HCDR3_consensus | WDYGGYFDV |
| | | Y F |
| 1224 | VL consensus | DIQMTQSPSSLSASVGDRVTITCRASQDIYNYLNWYQQKPG |
| | | KAVKLLIYYTSRLETGVPSRFSGSGSGTDYTLTISSLQPED |
| | | FATYFCQQGDTLPWTFGQGTKLEIK |
| 1225 | LCDR1_consensus | RASQDIYNYLN |
| | | FH |
| | | AS |
| | | S |
| 1226 | LCDR2_consensus | YTSRLET |
| | | M HS |
| | | G |
| | | I |
| | | S |
| | | E |
| 1227 | LCDR3_consensus | QQGDTLPWT |
| | | EAI |
| | | I |
| | | S |
| | | E |

TABLE 33
Consensus of 20E6 variants isolated from Doping Library after 2 hour
off-rate selection, P3 population

| Seq ID NO. | Identifier | Amino acid sequence |
|---|---|---|
| 1228 | VH consensus | EVQLVQSGAEVKKPGASVKVSCKASGYTFTSYLMQWVRQAP |
| | | GQGLEWMGRIDPQSGGIKYAQKFQGRATLTVDTSTSTAYME |
| | | LSRLRSDDTAVYYCARWDYGGYFDVWGQGTLVTVSS |
| 1229 | HCDR1_ onsensus | SYLMQ |
| | | R WLH |
| | | N DIN |
| | | VS |
| | | T |
| | | C |
| 1230 | HCDR2_consensus | RIDPQSGGIKYAQKFQG |
| | | EN DVI |
| | | RG SR |
| | | L TN |
| | | P N |
| | | D F |
| 1231 | HCDR3_ onsensus | WDYGGYFDV |
| | | NW F |
| | | F |
| | | H |
| 1232 | VL consensus | DIQMTQSPSSLSASVGDRVTITCRASQDIYNYLNWYQQKPG |
| | | KAVKLLIYYTSRLETGVPSRFSGSGSGTDYTLTISSLQPED |
| | | FATYFCQQGDTLPWTFGQGTKLEIK |
| 1233 | LCDR1_consensus | RASQDIYNYLN |
| | | FS |
| | | TY |
| | | A |
| | | S |
| 1234 | LCDR2_consensus | YTSRLET |
| | | S M HS |
| | | A G |
| | | S |
| | | E |
| 1235 | LCDR3_consensus | QQGDTLPWT |
| | | SV |
| | | A |
| | | I |

TABLE 34
Consensus of 20E6 variants isolated from Doping Library after 2 hour
off-rate selection, P4 population

| Seq ID NO. | Identifier | Amino acid sequence |
|---|---|---|
| 1236 | VH consensus | EVQLVQSGAEVKKPGASVKVSCKASGYTFTSYLMHWVRQAP |
| | | GQGLEWMGRIDPQSGGIKYAQKFQGRATLTVDTSTSTAYME |
| | | LSRLRSDDTAVYYCARWDYGGYFDVWGQGTLVTVSS |
| 1237 | HCDR1_consensus | SYLMH |
| | | R WLQ |
| | | T YVN |
| | | DTA |
| | | I |
| 1238 | HCDR2_consensus | RIDPQSGGIKYAQKFQG |
| | | LEG VSR |
| | | PT EVI |
| | | VN NT |
| | | R MM |
| | | M F |
| | | L |
| | | H |
| | | D |
| 1239 | HCDR3_consensus | WDYGGYFDV |
| | | W |
| | | H |
| 1240 | VL consensus | DIQMTQSPSSLSASVGDRVTITCRASQDITNYLNWYQQKPG |
| | | KAVKLLIYYTSRLETGVPSRFSGSGSGTDYTLTISSLQPED |
| | | FATYFCQQGDTLPWTFGQGTKLEIK |
| 1241 | LCDR1_consensus | RASQDITNYLN |
| | | YH |
| | | AK |
| 1242 | LCDR2_consensus | YTSRLET |
| | | S G HS |
| | | M |
| | | S |
| | | I |
| 1243 | LCDR3_consensus | QQGDTLPWT |
| | | I |
| | | A |

TABLE 35
Consensus of 20E6 variants isolated from Doping Library after 12 hour
off-rate selection

| Seq ID NO. | Identifier | Amino acid sequence |
|---|---|---|
| 1244 | VH consensus | EVQLVQSGAEVKKPGASVKVSCKASGYTFTSYYMNWVRQAP |
| | | GQGLEWMGRIDPQSGGIKYAQKFQGRATLTVDTSTSTAYME |
| | | LSRLRSDDTAVYYCARWDYGGYFDVWGQGTLVTVSS |
| 1245 | HCDR1_consensus | SYYMN |
| | | R LLQ |
| | | T WIH |
| | | G DVS |
| | | FT |
| 1246 | HCDR2_consensus | RIDPQSGGIKYAQKFQG |
| | | ED DVR |
| | | LI ETI |
| | | HN S |
| | | G N |
| | | L |
| 1247 | HCDR3_consensus | WDYGGYFDV |
| 1248 | VL consensus | DIQMTQSPSSLSASVGDRVTITCRASQDIWNYLNWYQQKPG |
| | | KAVKLLIYYTSMLETGVPSRFSGSGSGTDYTLTISSLQPED |
| | | FATYFCQQGDTLPWTFGQGTKLEIK |
| 1249 | LCDR1_consensus | RASQDIWNYLN |
| | | H |
| | | K |
| 1250 | LCDR2_consensus | YTSMLET |
| | | G HS |
| | | I |
| | | R |
| | | V |
| 1251 | LCDR3_consensus | QQGDTLPWT |
| | | S |
| | | N |
| | | A |
| | | M |
| | | I |

TABLE 36
Consensus of 20E6 variants isolated from TRIM library after 2 hour
off-rate selection, P1 population

| Seq ID NO. | Identifier | Amino acid sequence |
|---|---|---|
| 1252 | VH consensus | EVQLVQSGAEVKKPGASVKVSCKASGYTFTSYDMHWVRQAP |
| | | GQGLEWMGRIDPQSGGIKYAQKFQGRATLTVDTSTSTAYME |
| | | LSRLRSDDTAVYYCARWDYGGYFDVWGQGTLVTVSS |
| 1253 | HCDR1_consensus | SYDMH |
| | | R WLQ |
| | | Y Y |
| | | N |
| | | K |
| 1254 | HCDR2_consensus | RIDPQSGGIKYAQKFQG |
| | | DD DVR |
| | | FT T |
| | | N S |
| | | F |
| 1255 | HCDR3_consensus | WDYGGYFDV |
| | | Y F Q |
| 1256 | VL consensus | DIQMTQSPSSLSASVGDRVTITCRASQDIYNYLNWYQQKPG |
| | | KAVKLLIYYTSVLHSGVPSRESGSGSGTDYTLTISSLQPED |
| | | FATYFCQQGDTLPWTFGQGTKLEIK |
| 1257 | LCDR1_consensus | RASQDIYNYLN |
| | | K FK |
| 1258 | LCDR2_consensus | YTSVLHS |
| | | I |
| | | L |
| | | G |
| | | H |
| | | Y |
| | | N |
| 1259 | LCDR3_consensus | QQGDTLPWT |
| | | E |
| | | A |
| | | L |

TABLE 37
Consensus of 20E6 variants isolated from TRIM library after 2 hour
off-rate selection, P2 population

| Seq ID NO. | Identifier | Amino acid sequence |
|---|---|---|
| 1260 | VH consensus | EVQLVQSGAEVKKPGASVKVSCKASGYTFTSYDMHWVRQAP |
| | | GQGLEWMGRIDPQSGGIKYAQKFQGRATLTVDTSTSTAYME |
| | | LSRLRSDDTAVYYCARWDYGGYFDVWGQGTLVTVSS |
| 1261 | HCDR1_consensus | SYDMH |
| | | Y LN |
| | | R |
| | | Q |
| | | N |
| 1262 | HCDR2_consensus | RIDPQSGGIKYAQKFQG |
| | | D DVR |
| | | T T |
| | | V Q |
| 1263 | HCDR3_consensus | WDYGGYFDV |
| | | Y Q |
| | | F |
| 1264 | VL consensus | DIQMTQSPSSLSASVGDRVTITCRASQDIYNYLNWYQQKPG |
| | | KAVKLLIYYTSRLHSGVPSRFSGSGSGTDYTLTISSLQPED |
| | | FATYFCQQGDTLPWTFGQGTKLEIK |
| 1265 | LCDR1_consensus | RASQDIYNYLN |
| | | FK |
| 1266 | LCDR2_consensus | YTSRLHS |
| | | G |
| | | I |
| | | V |
| | | L |
| | | S |
| | | E |
| 1267 | LCDR3_consensus | QQGDTLPWT |
| | | EI |
| | | G |
| | | D |
| | | A |

TABLE 38
Consensus of 20E6 variants isolated from TRIM library after 2 hour
off-rate selection, P3 population

| Seq ID NO. | Identifier | Amino acid sequence |
|---|---|---|
| 1268 | VH consensus | EVQLVQSGAEVKKPGASVKVSCKASGYTFTSYDMHWVRQAP |
| | | GQGLEWMGRIDPQSGGIKYAQKFQGRATLTVDTSTSTAYME |
| | | LSRLRSDDTAVYYCARWDYGGYFDVWGQGTLVTVSS |
| 1269 | HCDR1_consensus | SYDMH |
| | | F WIS |
| | | L |
| | | V |
| 1270 | HCDR2_consensus | RIDPQSGGIKYAQKFQG |
| | | N DTV |
| | | T DR |
| | | G S |
| 1271 | HCDR3_consensus | WDYGGYFDV |
| | | YEHA |
| | | Q |
| 1272 | VL consensus | DIQMTQSPSSLSASVGDRVTITCRASQDIYNYLNWYQQKPG |
| | | KAVKLLIYYTSRLHSGVPSRFSGSGSGTDYTLTISSLQPED |
| | | FATYFCQQGDTLPWTFGQGTKLEIK |
| 1273 | LCDR1_consensus | RASQDIYNYLN |
| | | G TY |
| | | F |
| 1274 | LCDR2_consensus | YTSRLHS |
| | | V |
| | | N |
| | | Y |
| | | I |
| | | H |
| | | A |
| 1275 | LCDR3_consensus | QQGDTLPWT |

TABLE 39
Consensus of 20E6 variants isolated from TRIM library after 2 hour
off-rate selection, P4 population

| Seq ID NO. | Identifier | Amino acid sequence |
|---|---|---|
| 1276 | VH consensus | EVQLVQSGAEVKKPGASVKVSCKASGYTFTSYDMHWVRQAP |
| | | GQGLEWMGRIDPQSGGIKYAQKFQGRATLTVDTSTSTAYME |
| | | LSRLRSDDTAVYYCARWDYGGYFDVWGQGTLVTVSS |
| 1277 | HCDR1_consensus | SYDMH |
| | | D WLQ |
| | | T QVV |
| | | R EIS |
| | | P F |
| | | N |
| | | H |
| | | G |
| | | A |
| 1278 | HCDR2_consensus | RIDPQSGGIKYAQKFQG |
| | | W YG SYR |
| | | VR DVI |
| | | K NHP |
| | | I LD |
| | | D DA |
| 1279 | HCDR3_consensus | WDYGGYFDV |
| | | Y HS N |
| | | QA L |
| | | E F |
| 1280 | VL consensus | DIQMTQSPSSLSASVGDRVTITCRASQDIYNYLNWYQQKPG |
| | | KAVKLLIYYTSRLHSGVPSRFSGSGSGTDYTLTISSLQPED |
| | | FATYFCQQGDTLPWTFGQGTKLEIK |
| 1281 | LCDR1_consensus | RASQDIYNYLN |
| | | TS |
| | | FY |
| | | H |
| | | K |
| 1282 | LCDR2_consensus | YTSRLHS |
| | | N |
| | | Q |
| | | I |
| | | E |
| | | A |
| 1283 | LCDR3_consensus | QQGDTLPWT |
| | | Q |
| | | V |

TABLE 40
Consensus of 20E6 variants isolated from TRIM library after 12 hour
off-rate selection

| Seq ID NO. | Identifiter | Amino acid sequence |
|---|---|---|
| 1284 | VH consensus | EVQLVQSGAEVKKPGASVKVSCKASGYTFTSYDMHWVRQAP |
| | | GQGLEWMGRIDPQSGGIKYAQKFQGRATLTVDTSTSTAYME |
| | | LSRLRSDDTAVYYCARWDYGGYFDVWGQGTLVTVSS |
| 1285 | HCDR1_consensus | SYDMH |
| | | H YLN |
| | | Y LIQ |
| | | Q |
| 1286 | HCDR2_consensus | RIDPQSGGIKYAQKFQG |
| | | DD DTR |
| | | VT SV |
| | | IG S |
| | | Y |
| | | P |
| 1287 | HCDR3_consensus | WDYGGYFDV |
| | | HT Q |
| | | H |
| 1288 | VL consensus | DIQMTQSPSSLSASVGDRVTITCRASQDIYNYLNWYQQKPG |
| | | KAVKLLIYYTSILHSGVPSRFSGSGSGTDYTLTISSLQPED |
| | | FATYFCQQGDTLPWTFGQGTKLEIK |
| 1289 | LCDR1_consensus | RASQDIYNYLN |
| | | FS |
| | | WK |
| 1290 | LCDR2_consensus | YTSILHS |
| | | R |
| | | H |
| | | V |
| | | G |
| | | N |
| | | E |
| 1291 | LCDR3_consensus | QQGDTLPWT |
| | | EI |
| | | A |

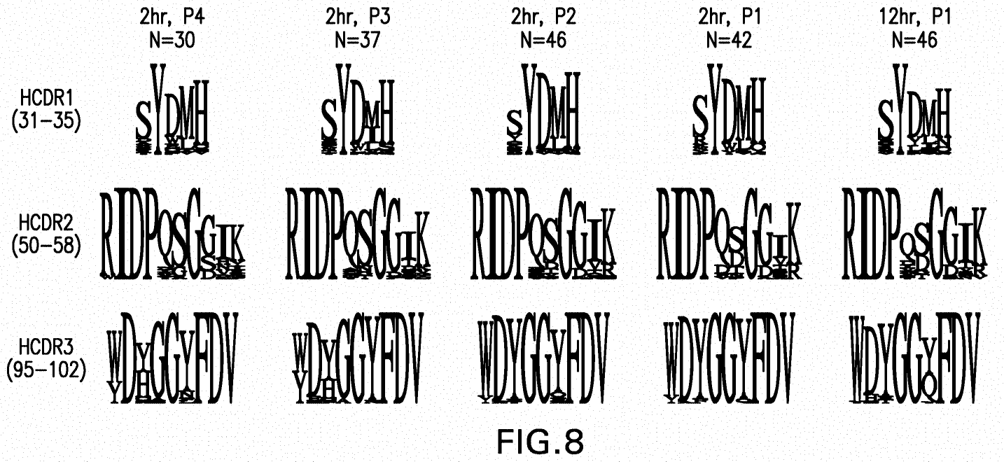

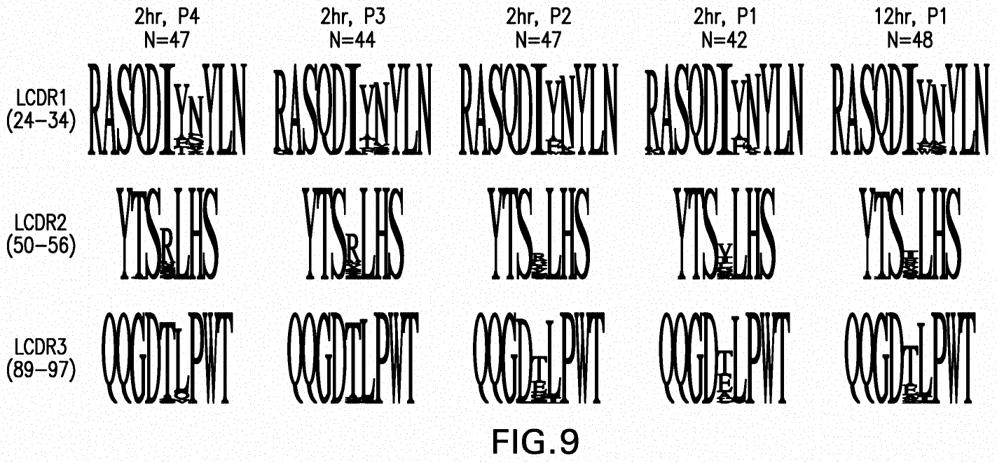

Sequence logos were next prepared to better visualize the frequency distribution of CDR residue changes in 20E6 variants from the various sorting conditions for doping (FIG. 6 and FIG. 7) and TRIM (FIG. 8 and FIG. 9) library selection outputs. While the distributions of different amino acids at each CDR residue appear to be similar across the different selection conditions within the same library, there are surprising differences between the doping and TRIM libraries outputs. For example, in the VH the prevalent S3IR change only appeared in the doping library outputs; the W-33 D change is more dominant in the TRIM library outputs; and the Y100Q change is prominent only in the TRIM library after 12 hours of off-rate selection. In the VL, the dominant T30Y change in the TRIM and doping library outputs P2 to P4 was replaced by T30W change with greater off-rate selection pressure in the doping library P1 outputs at 2 and 12 hours. Lastly, the VL CDR2 H55/S56 to E55/T56 human germline changes appeared to be preferred in the doping library output upon off-rate selection pressure. These changes were missing in the TRIM library output as they were not built in the TRIM library. The E55/T56 germline changes were incorporated into the TRIM library output sequences.

### Example 10: Analysis of Affinity Matured 20E6 Variant Sequences from all Doping and TRIM Library Selection Outputs

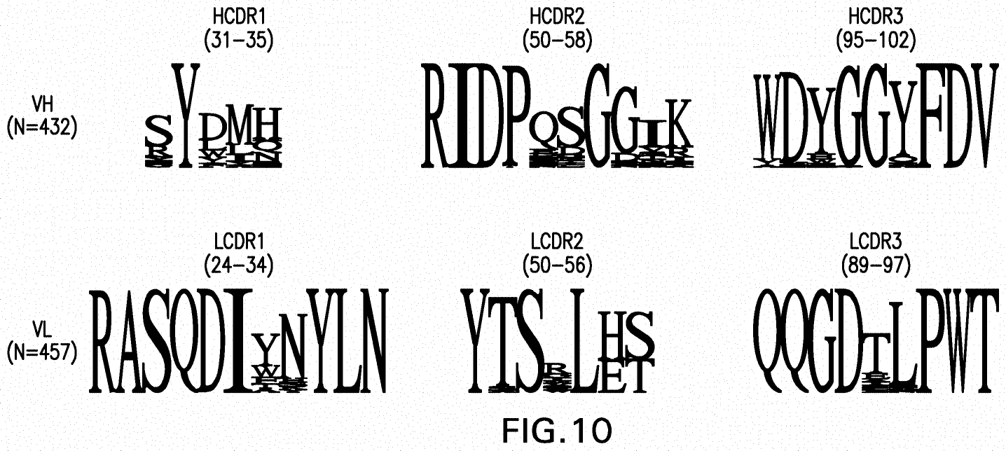

To provide a complete overview of CDR residues that contribute to improved affinity to LAP-TGFb1, the investigators aligned all available 432 VH and 457 VL sequences from all selection outputs from both the doping and TRIM libraries. The sequence logos shown in FIG. 10 highlight the amino acid compositions at each position and their relative representations. Table 41 summarizes the consensus sequences of 20E6 variants from all outputs as well as amino acids that are represented at more than 1% of the available sequences in the order of abundance at each CDR residue positions. It is expected that each identified amino acid change in the 20E6 CDRs alone or in combinations will contribute to improved affinity to human LAP-TGFb1.

TABLE 41
Consensus of 20E6 variants isolated from both TRIM and doping libraries
after all selection conditions

| Seq ID | Identifier | Amino acid sequence |
|---|---|---|
| 2217 | VH consensus | EVQLVQSGAEVKKPGASVKVSCKASGYTFTSYDMHWVRQAP |
| | | GQGLEWMGRIDPQSGGIKYAQKFQGRATLTVDTSTSTAYME |
| | | LSRLRSDDTAVYYCARWDYGGYFDVWGQGTLVTVSS |
| 2218 | HCDR1_consensus | SYDMH |
| | | R LLQ |
| | | N WIN |
| | | Y YVS |
| | | T FTA |
| | | Q T |
| | | K |
| | | H |
| | | G |
| | | A |
| 2219 | HCDR2_consensus | RIDPQSGGIKYAQKFQG |
| | | ED DVR |
| | | DG SSI |
| | | LT ETL |
| | | PN F |
| | | VI N |
| | | YR G |
| | | I D |
| | | H M |
| | | R |
| 2220 | HCDR3_consensus | WDYGGYFDV |
| | | YHH Q |
| | | W F |
| | | F |
| 2221 | VL consensus | DIQMTQSPSSLSASVGDRVTITCRASQDIYNYLNWYQQKPG |
| | | KAVKLLIYYTSRLHSGVPSRFSGSGSGTDYTLTISSLQPED |
| | | FATYFCQQGDTLPWTFGQGTKLEIK |
| 2222 | LCDR1_consensus | RASQDIYNYLN |
| | | WH |
| | | FS |
| | | TK |
| | | AY |
| | | S |
| 2223 | LCDR2_consensus | YTSRLHS |
| | | S G ET |
| | | I |
| | | M |
| | | V |
| | | S |
| | | N |
| | | L |
| | | H |
| | | E |
| | | Y |
| | | Q |
| | | K |
| | | A |
| 2224 | LCDR3_consensus | QQGDTLPWT |
| | | EI |
| | | AV |
| | | IQ |
| | | S |
| | | M |
| | | L |
| | | G |
| | | N |
| | | D |

### Example 11: Next Generation Sequencing (NGS) Analysis of 20E6 Variants from Yeast Selection Outputs

To more comprehensively understand the CDR residue changes that are selected from the different sorting gates for better binding to LAP-TGFb1 protein by yeast display, the investigators isolated 20E6 variant sequences from each of the output and subjected them to next generation sequences. Without being limited, it is believed that the sequences derived from the NGS analysis provide additional information on critical residues at each CDR position that contributed to improved LAP-TGFb1 binding individually or in combination. The increased available sequences allowed the selection of 20E6 variants that have the most optimal binding to LAP-TGFb1 while minimizing impact on potentially increased immunogenicity or developability.

### Example 12: Expression of 20E6 Variants from Different Off-Rate Selection Pressure as Fab Proteins for Binding Analysis

Twenty-seven affinity matured 20E6 variants from the five TRIM library outputs were converted to soluble Fab proteins for recombinant expression by CHO cells for binding analysis. The sequences are listed in Table 42 and Table 43.

TABLE 42
20E6 variants isolated from yeast display selection

| Seq | | |
|---|---|---|
| ID | | |
| NO. | Identifier | Amino acid sequence |
| 1292 | VH W33D M34I | SYDIH |
| | (55BJN)_HCDR1 | |
| 1293 | VH W33D M34I | RIDPQSGGIKYAQKFQG |
| | (55BJN)_HCDR2 | |
| 1294 | VH W33D M34I | WDYGGYFDV |
| | (55BJN)_HCDR3 | |
| 1295 | VH W33D M34I | EVOLVQSGAEVKKPGASVKVSCKASGYTFTSYDIHWVRQA |
| | (55BJN)_VH | PGOGLEWMGRIDPQSGGIKYAQKFQGRATLTVDTSTSTAY |
| | | MELSRLRSDDTAVYYCARWDYGGYFDVWGQGTLVTVSS |
| 1296 | VH W33D M34I | EVQLVQSGAEVKKPGASVKVSCKASGYTFTSYDIHWVRQA |
| | (55BJN)_Fab_HC | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1297 | VL (55BJN)_LCDR1 | RASQDITNYLN |
| 1298 | VL (55BJN)_LCDR2 | YTSRLHS |
| 1299 | VL (55BJN)_LCDR3 | QQGDTLPWT |
| 1300 | VL (55BJN)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIK |
| 1301 | VL (55BJN)_LC | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1302 | VH W33D M34I Q54Y | SYDIH |
| | (56BJN)_HCDR1 | |
| 1303 | VH W33D M34I Q54Y | RIDPYSGGIKYAQKFQG |
| | (56BJN)_HCDR2 | |
| 1304 | VH W33D M34I Q54Y | WDYGGYFDV |
| | (56BJN)_HCDR3 | |
| 1305 | VH W33D M34I Q54Y | EVQLVQSGAEVKKPGASVKVSCKASGYTFTSYDIHWVRQA |
| | (56BJN)_VH | PGQGLEWMGRIDPYSGGIKYAQKFQGRATLTVDTSTSTAY |
| | | MELSRLRSDDTAVYYCARWDYGGYFDVWGQGTLVTVSS |
| 1306 | VH W33D M34I Q54Y | EVQLVQSGAEVKKPGASVKVSCKASGYTFTSYDIHWVRQA |
| | (56BJN)_Fab_HC | PGQGLEWMGRIDPYSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1307 | VL (56BJN)_LCDR1 | RASQDITNYLN |
| 1308 | VL (56BJN)_LCDR2 | YTSRLHS |
| 1309 | VL (56BJN)_LCDR3 | QQGDTLPWT |
| 1310 | VL (56BJN)_VL | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIK |
| 1311 | VL (56BJN)_LC | DIQMTQSPSSLSASVGDRVTITCRASQDITNYLNWYQQKP |
| | | GKAVKLLIYYTSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1312 | VH W33D | SYDMH |
| | (57BJN)_HCDR1 | |
| 1313 | VH W33D | RIDPQSGGIKYAQKFQG |
| | (57BJN)_HCDR2 | |
| 1314 | VH W33D | WDYGGYFDV |
| | (57BJN)_HCDR3 | |
| 1315 | VH W33D (57BJN)_VH | EVOLVQSGAEVKKPGASVKVSCKASGYTFTSYDMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 1316 | VH W33D | EVQLVQSGAEVKKPGASVKVSCKASGYTFTSYDMHWVRQA |
| | (57BJN)_Fab_HC | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1317 | VL (57BJN)_LCDR1 | RASQDITNYLN |
| 1318 | VL (57BJN)_LCDR2 | YTSRLHS |
| 1319 | VL (57BJN)_LCDR3 | QQGDTLPWT |
| 1320 | VL (57BJN)_VL | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIK |
| 1321 | VL (57BJN)_LC | DIQMTQSPSSLSASVGDRVTITCRASQDITNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1322 | VH W33D W99Y Y101H | SYDMH |
| | (58BJN)_HCDR1 | |
| 1323 | VH W33D W99Y Y101H | RIDPQSGGIKYAQKFQG |
| | (58BJN)_HCDR2 | |
| 1324 | VH W33D W99Y Y101H | YDHGGYFDV |
| | (58BJN)_HCDR3 | |
| 1325 | VH W33D W99Y Y101H | EVQLVQSGAEVKKPGASVKVSCKASGYTFTSYDMHWVRQA |
| | (58BJN)_VH | PGQGLEWMGRIDPQSGGIKYAQKFQGRATLTVDTSTSTAY |
| | | MELSRLRSDDTAVYYCARYDHGGYFDVWGQGTLVTVSS |
| 1326 | VH W33D W99Y Y101H | EVQLVQSGAEVKKPGASVKVSCKASGYTFTSYDMHWVRQA |
| | (58BJN)_Fab_HC | PGQGLEWMGRIDPQSGGIKYAQKFQGRATLTVDTSTSTAY |
| | | MELSRLRSDDTAVYYCARYDHGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1327 | VL T30Y | RASQDIYNYLN |
| | (58BJN)_LCDR1 | |
| 1328 | VL T30Y | YTSRLHS |
| | (58BJN)_LCDR2 | |
| 1329 | VL T30Y | QQGDTLPWT |
| | (58BJN)_LCDR3 | |
| 1330 | VL T30Y (58BJN)_VL | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIK |
| 1331 | VL T30Y (58BJN)_LC | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1332 | VH W33Q M34V | SYQVH |
| | (59BJN)_HCDR1 | |
| 1333 | VH W33Q M34V | RIDPQSGGIKYAQKFQG |
| | (59BJN)_HCDR2 | |
| 1334 | VH W33Q M34V | DYGGYFDV |
| | (59BJN)_HCDR3 | |
| 1335 | VH W33Q M34V | EVQLVQSGAEVKKPGASVKVSCKASGYTFTSYQVHWVRQA |
| | (59BJN)_VH | PGQGLEWMGRIDPQSGGIKYAQKFQGRATLTVDTSTSTAY |
| | | MELSRLRSDDTAVYYCARWDYGGYFDVWGQGTLVTVSS |
| 1336 | VH W33Q M34V | EVOLVQSGAEVKKPGASVKVSCKASGYTFTSYQVHWVRQA |
| | (59BJN)_Fab_HC | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1337 | VL T30Y | RASQDIYNYLN |
| | (59BJN)_LCDR1 | |
| 1338 | VL T30Y | YTSRLHS |
| | (59BJN)_LCDR2 | |
| 1339 | VL T30Y | QQGDTLPWT |
| | (59BJN)_LCDR3 | |
| 1340 | VL T30Y (59BJN)_VL | DIQMTQSPSSLSASVGDRVTITCRASQDIYNYLNWYQQKP |
| | | GKAVKLLIYYTSRLHSGVPSRESGSGSGTDYTLTISSLQP |
| | | EDFATYFCQQGDTLPWTFGQGTKLEIK |
| 1341 | VL T30Y (59BJN)_LC | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1342 | VH W33D I58E W99Y | SYDMH |
| | (60BJN)_HCDR1 | |
| 1343 | VH W33D I58E W99Y | RIDPQSGGEKYAQKFQG |
| | (60BJN)_HCDR2 | |
| 1344 | VH W33D I58E W99Y | YDYGGYFDV |
| | (60BJN)_HCDR3 | |
| 1345 | VH W33D I58E W99Y | EVQLVQSGAEVKKPGASVKVSCKASGYTFTSYDMHWVRQA |
| | (60BJN)_VH | PGQGLEWMGRIDPQSGGEKYAQKFQGRATLTVDTSTSTAY |
| | | MELSRLRSDDTAVYYCARYDYGGYFDVWGQGTLVTVSS |
| 1346 | VH W33D I58E W99Y | EVOLVQSGAEVKKPGASVKVSCKASGYTFTSYDMHWVRQA |
| | (60BJN)_Fab_HC | PGQGLEWMGRIDPQSGGEKYAQKFQGRATLTVDTSTSTAY |
| | | MELSRLRSDDTAVYYCARYDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1347 | VL T30Y | RASQDIYNYLN |
| | (60BJN)_LCDR1 | |
| 1348 | VL T30Y | YTSRLHS |
| | (60BJN)_LCDR2 | |
| 1349 | VL T30Y | QQGDTLPWT |
| | (60BJN)_LCDR3 | |
| 1350 | VL T30Y (60BJN)_VL | DIQMTQSPSSLSASVGDRVTITCRASQDIYNYLNWYQQKP |
| | | GKAVKLLIYYTSRLHSGVPSRESGSGSGTDYTLTISSLQP |
| | | EDFATYFCQQGDTLPWTFGQGTKLEIK |
| 1351 | VL T30Y (60BJN)_LC | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 1352 | VH S31D W33D G57D | DYDMH |
| | I58H K59D W99Y Y101H | |
| | (61BJN)_HCDR1 | |
| 1353 | VH S31D W33D G57D | RIDPQSGDHDYAQKFQG |
| | I58H K59D W99Y Y101H | |
| | (61BJN)_HCDR2 | |
| 1354 | VH S31D W33D G57D | YDHGGYFDV |
| | I58H K59D W99Y Y101H | |
| | (61BJN)_HCDR3 | |
| 1355 | VH S31D W33D G57D | EVOLVQSGAEVKKPGASVKVSCKASGYTFTDYDMHWVRQA |
| | I58H K59D W99Y Y101H | PGQGLEWMGRIDPQSGDHDYAQKFQGRATLTVDTSTSTAY |
| | (61BJN)_VH | MELSRLRSDDTAVYYCARYDHGGYFDVWGQGTLVTVSS |
| 1356 | VH S31D W33D G57D | EVQLVQSGAEVKKPGASVKVSCKASGYTFTDYDMHWVRQA |
| | I58H K59D W99Y Y101H | PGQGLEWMGRIDPQSGDHDYAQKFQGRATLTVDTSTSTAY |
| | (61BJN)_Fab_HC | MELSRLRSDDTAVYYCARYDHGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1357 | VL T30Y | RASQDIYNYLN |
| | (61BJN)_LCDR1 | |
| 1358 | VL T30Y | YTSRLHS |
| | (61BJN)_LCDR2 | |
| 1359 | VL T30Y | QQGDTLPWT |
| | (61BJN)_LCDR3 | |
| 1360 | VL T30Y (61BJN)_VL | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIK |
| 1361 | VL T30Y (61BJN)_LC | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1362 | VH W33D Q54Y S55G | SYDMH |
| | W99Y Y101H | |
| | (62BJN)_HCDR1 | |
| 1363 | VH W33D Q54Y S55G | RIDPYGGGIKYAQKFQG |
| | W99Y Y101H | |
| | (62BJN)_HCDR2 | |
| 1364 | VH W33D Q54Y S55G | YDHGGYFDV |
| | W99Y Y101H | |
| | (62BJN)_HCDR3 | |
| 1365 | VH W33D Q54Y S55G | EVOLVQSGAEVKKPGASVKVSCKASGYTFTSYDMHWVRQA |
| | W99Y Y101H | PGQGLEWMGRIDPYGGGIKYAQKFQGRATLTVDTSTSTAY |
| | (62BJN)_VH | MELSRLRSDDTAVYYCARYDHGGYFDVWGQGTLVTVSS |
| 1366 | VH W33D Q54Y S55G | EVQLVQSGAEVKKPGASVKVSCKASGYTFTSYDMHWVRQA |
| | W99Y Y101H | PGQGLEWMGRIDPYGGGIKYAQKFQGRATLTVDTSTSTAY |
| | (62BJN)_Fab_HC | MELSRLRSDDTAVYYCARYDHGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1367 | VL T30Y | RASQDIYNYLN |
| | (62BJN)_LCDR1 | |
| 1368 | VL T30Y | YTSRLHS |
| | (62BJN)_LCDR2 | |
| 1369 | VL T30Y | QQGDTLPWT |
| | (62BJN)_LCDR3 | |
| 1370 | VL T30Y (62BJN)_VL | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIK |
| 1371 | VL T30Y (62BJN)_LC | DIQMTQSPSSLSASVGDRVTITCRASQDIYNYLNWYQQKP |
| | | GKAVKLLIYYTSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 1372 | VH S31F W33D M34I | FYDIH |
| | (63BJN)_HCDR1 | |
| 1373 | VH S31F W33D M34I | RIDPQSGGIKYAQKFQG |
| | (63BJN)_HCDR2 | |
| 1374 | VH S31F W33D M34I | WDYGGYFDV |
| | (63BJN)_HCDR3 | |
| 1375 | VH S31F W33D M34I | EVQLVQSGAEVKKPGASVKVSCKASGYTFTFYDIHWVRQA |
| | (63BJN)_VH | PGQGLEWMGRIDPQSGGIKYAQKFQGRATLTVDTSTSTAY |
| | | MELSRLRSDDTAVYYCARWDYGGYFDVWGQGTLVTVSS |
| 1376 | VH S31F W33D M34I | EVQLVQSGAEVKKPGASVKVSCKASGYTFTFYDIHWVRQA |
| | (63BJN)_Fab_HC | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1377 | VL T30Y | RASQDIYNYLN |
| | (63BJN)_LCDR1 | |
| 1378 | VL T30Y | YTSRLHS |
| | (63BJN)_LCDR2 | |
| 1379 | VL T30Y | QQGDTLPWT |
| | (63BJN)_LCDR3 | |
| 1380 | VL T30Y (63BJN)_VL | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIK |
| 1381 | VL T30Y (63BJN)_LC | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1382 | VH S31T I58S | TYWMH |
| | (64BJN)_HCDR1 | |
| 1383 | VH S31T I58S | RIDPQSGGSKYAQKFQG |
| | (64BJN)_HCDR2 | |
| 1384 | VH S31T I58S | WDYGGYFDV |
| | (64BJN)_HCDR3 | |
| 1385 | VH S31T I58S | EVQLVQSGAEVKKPGASVKVSCKASGYTFTTYWMHWVRQA |
| | (64BJN)_VH | PGQGLEWMGRIDPQSGGSKYAQKFQGRATLTVDTSTSTAY |
| | | MELSRLRSDDTAVYYCARWDYGGYFDVWGQGTLVTVSS |
| 1386 | VH S31T I58S | EVOLVQSGAEVKKPGASVKVSCKASGYTFTTYWMHWVRQA |
| | (64BJN)_Fab_HC | PGQGLEWMGRIDPQSGGSKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1387 | VL T30Y R53H | RASQDIYNYLN |
| | (64BJN)_LCDR1 | |
| 1388 | VL T30Y R53H | YTSHLHS |
| | (64BJN)_LCDR2 | |
| 1389 | VL T30Y R53H | QQGDTLPWT |
| | (64BJN)_LCDR3 | |
| 1390 | VL T30Y R53H | DIQMTQSPSSLSASVGDRVTITCRASQDIYNYLNWYQQKP |
| | (64BJN)_VL | GKAVKLLIYYTSHLHSGVPSRFSGSGSGTDYTLTISSLQP |
| | | EDFATYFCQQGDTLPWTFGQGTKLEIK |
| 1391 | VL T30Y R53H | DIQMTQSPSSLSASVGDRVTITCRASQDIYNYLNWYQQKP |
| | (64BJN)_LC | GKAVKLLIYYTSHLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1392 | VH W33D M34L W99Y | SYDLH |
| | Y101Q (65BJN)_HCDR1 | |
| 1393 | VH W33D M34L W99Y | RIDPQSGGIKYAQKFQG |
| | Y101Q (65BJN)_HCDR2 | |
| 1394 | VH W33D M34L W99Y | YDQGGYFDV |
| | Y101Q (65BJN)_HCDR3 | |
| 1395 | VH W33D M34L W99Y | EVQLVQSGAEVKKPGASVKVSCKASGYTFTSYDLHWVRQA |
| | Y101Q (65BJN)_VH | PGQGLEWMGRIDPQSGGIKYAQKFQGRATLTVDTSTSTAY |
| | | MELSRLRSDDTAVYYCARYDQGGYFDVWGQGTLVTVSS |
| 1396 | VH W33D M34L W99Y | EVOLVQSGAEVKKPGASVKVSCKASGYTFTSYDLHWVRQA |
| | Y101Q (65BJN)_Fab_HC | PGQGLEWMGRIDPQSGGIKYAQKFQGRATLTVDTSTSTAY |
| | | MELSRLRSDDTAVYYCARYDOGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1397 | VL T30Y N31Y | RASQDIYYYLN |
| | (65BJN)_LCDR1 | |
| 1398 | VL T30Y N31Y | YTSRLHS |
| | (65BJN)_LCDR2 | |
| 1399 | VL T30Y N31Y | QQGDTLPWT |
| | (65BJN)_LCDR3 | |
| 1400 | VL T30Y N31Y | DIQMTQSPSSLSASVGDRVTITCRASQDIYYYLNWYQQKP |
| | (65BJN)_VL | GKAVKLLIYYTSRLHSGVPSRESGSGSGTDYTLTISSLQP |
| | | EDFATYFCQQGDTLPWTFGQGTKLEIK |
| 1401 | VL T30Y N31Y | DIQMTQSPSSLSASVGDRVTITCRASQDIYYYLNWYQQKP |
| | (65BJN)_LC | GKAVKLLIYYTSRLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 1402 | VH W33D S55T Y101H | SYDMH |
| | (66BJN)_HCDR1 | |
| 1403 | VH W33D S55T Y101H | RIDPQTGGIKYAQKFQG |
| | (66BJN)_HCDR2 | |
| 1404 | VH W33D S55T Y101H | WDHGGYFDV |
| | (66BJN)_HCDR3 | |
| 1405 | VH W33D S55T Y101H | EVOLVQSGAEVKKPGASVKVSCKASGYTFTSYDMHWVRQA |
| | (66BJN)_VH | PGQGLEWMGRIDPQTGGIKYAQKFQGRATLTVDTSTSTAY |
| | | MELSRLRSDDTAVYYCARWDHGGYFDVWGQGTLVTVSS |
| 1406 | VH W33D S55T Y101H | EVOLVQSGAEVKKPGASVKVSCKASGYTFTSYDMHWVRQA |
| | (66BJN)_Fab_HC | PGQGLEWMGRIDPQTGGIKYAQKFQGRATLTVDTSTSTAY |
| | | MELSRLRSDDTAVYYCARWDHGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1407 | VL T30Y L94Q | RASQDIYNYLN |
| | (66BJN)_LCDR1 | |
| 1408 | VL T30Y L94Q | YTSRLHS |
| | (66BJN)_LCDR2 | |
| 1409 | VL T30Y L94Q | QQGDTQPWT |
| | (66BJN)_LCDR3 | |
| 1410 | VL T30Y L94Q | DIQMTQSPSSLSASVGDRVTITCRASQDIYNYLNWYQQKP |
| | (66BJN)_VL | GKAVKLLIYYTSRLHSGVPSRFSGSGSGTDYTLTISSLQP |
| | | EDFATYFCQQGDTQPWTFGQGTKLEIK |
| 1411 | VL T30Y L94Q | DIQMTQSPSSLSASVGDRVTITCRASQDIYNYLNWYQQKP |
| | (66BJN)_LC | GKAVKLLIYYTSRLHSGVPSRESGSGSGTDYTLTISSLQP |
| | | EDFATYFCQQGDTQPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1412 | VH W33D | SYDMH |
| | (67BJN)_HCDR1 | |
| 1413 | VH W33D | RIDPQSGGIKYAQKFQG |
| | (67BJN)_HCDR2 | |
| 1414 | VH W33D | WDYGGYFDV |
| | (67BJN)_HCDR3 | |
| 1415 | VH W33D (67BJN)_VH | EVOLVQSGAEVKKPGASVKVSCKASGYTFTSYDMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 1416 | VH W33D | EVQLVQSGAEVKKPGASVKVSCKASGYTFTSYDMHWVRQA |
| | (67BJN)_Fab_HC | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1417 | VL T30Y R53V | RASQDIYNYLN |
| | (67BJN)_LCDR1 | |
| 1418 | VL T30Y R53V | YTSVLHS |
| | (67BJN)_LCDR2 | |
| 1419 | VL T30Y R53V | QQGDTLPWT |
| | (67BJN)_LCDR3 | |
| 1420 | VL T30Y R53V | DIQMTQSPSSLSASVGDRVTITCRASQDIYNYLNWYQQKP |
| | (67BJN)_VL | GKAVKLLIYYTSVLHSGVPSRESGSGSGTDYTLTISSLQP |
| | | EDFATYFCQQGDTLPWTFGQGTKLEIK |
| 1421 | VL T30Y R53V | DIQMTQSPSSLSASVGDRVTITCRASQDIYNYLNWYQQKP |
| | (67BJN)_LC | GKAVKLLIYYTSVLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 1422 | VH H35Q S55D S57D | SYWMQ |
| | W99Y (68BJN)_HCDR1 | |
| 1423 | VH H35Q S55D S57D | RIDPQDGDIKYAQKFQG |
| | W99Y (68BJN)_HCDR2 | |
| 1424 | VH H35Q S55D S57D | YDYGGYFDV |
| | W99Y (68BJN)_HCDR3 | |
| 1425 | VH H35Q S55D S57D | EVQLVQSGAEVKKPGASVKVSCKASGYTFTSYWMQWVRQA |
| | W99Y (68BJN)_VH | PGQGLEWMGRIDPQDGDIKYAQKFQGRATLTVDTSTSTAY |
| | | MELSRLRSDDTAVYYCARYDYGGYFDVWGQGTLVTVSS |
| 1426 | VH H35Q S55D S57D | EVQLVQSGAEVKKPGASVKVSCKASGYTFTSYWMQWVRQA |
| | W99Y (68BJN)_Fab_HC | PGQGLEWMGRIDPQDGDIKYAQKFQGRATLTVDTSTSTAY |
| | | MELSRLRSDDTAVYYCARYDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1427 | VL T30Y R53V | RASQDIYNYLN |
| | (68BJN)_LCDR1 | |
| 1428 | VL T30Y R53V | YTSVLHS |
| | (68BJN)_LCDR2 | |
| 1429 | VL T30Y R53V | QQGDTLPWT |
| | (68BJN)_LCDR3 | |
| 1430 | VL T30Y R53V | DIQMTQSPSSLSASVGDRVTITCRASQDIYNYLNWYQQKP |
| | (68BJN)_VL | GKAVKLLIYYTSVLHSGVPSRFSGSGSGTDYTLTISSLQP |
| | | EDFATYFCQQGDTLPWTFGQGTKLEIK |
| 1431 | VL T30Y R53V | DIQMTQSPSSLSASVGDRVTITCRASQDIYNYLNWYQQKP |
| | (68BJN)_LC | GKAVKLLIYYTSVLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1432 | VH W33D I58T | SYDMH |
| | (69BJN)_HCDR1 | |
| 1433 | VH W33D I58T | RIDPQSGGTKYAQKFQG |
| | (69BJN)_HCDR2 | |
| 1434 | VH W33D I58T | WDYGGYFDV |
| | (69BJN)_HCDR3 | |
| 1435 | VH W33D I58T | EVQLVQSGAEVKKPGASVKVSCKASGYTFTSYDMHWVRQA |
| | (69BJN)_VH | PGQGLEWMGRIDPQSGGTKYAQKFQGRATLTVDTSTSTAY |
| | | MELSRLRSDDTAVYYCARWDYGGYFDVWGQGTLVTVSS |
| 1436 | VH W33D I58T | EVQLVQSGAEVKKPGASVKVSCKASGYTFTSYDMHWVRQA |
| | (69BJN)_Fab_HC | PGQGLEWMGRIDPQSGGTKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1437 | VL T30Y R53V | RASQDIYNYLN |
| | (69BJN)_LCDR1 | |
| 1438 | VL T30Y R53V | YTSVLHS |
| | (69BJN)_LCDR2 | |
| 1439 | VL T30Y R53V | QQGDTLPWT |
| | (69BJN)_LCDR3 | |
| 1440 | VL T30Y R53V | DIQMTQSPSSLSASVGDRVTITCRASQDIYNYLNWYQQKP |
| | (69BJN)_VL | GKAVKLLIYYTSVLHSGVPSRFSGSGSGTDYTLTISSLQP |
| | | EDFATYFCQQGDTLPWTFGQGTKLEIK |
| 1441 | VL T30Y R53V | DIQMTQSPSSLSASVGDRVTITCRASQDIYNYLNWYQQKP |
| | (69BJN)_LC | GKAVKLLIYYTSVLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1442 | VH W33D S55T G57D | SYDMH |
| | Y104Q (71BJN)_HCDR1 | |
| 1443 | VH W33D S55T G57D | RIDPQTGDIKYAQKFQG |
| | Y104Q (71BJN)_HCDR2 | |
| 1444 | VH W33D S55T G57D | WDYGGQFDV |
| | Y104Q (71BJN)_HCDR3 | |
| 1445 | VH W33D S55T G57D | EVQLVQSGAEVKKPGASVKVSCKASGYTFTSYDMHWVRQA |
| | Y104Q (71BJN)_VH | PGQGLEWMGRIDPQTGDIKYAQKFQGRATLTVDTSTSTAY |
| | | MELSRLRSDDTAVYYCARWDYGGQFDVWGQGTLVTVSS |
| 1446 | VH W33D S55T G57D | EVOLVQSGAEVKKPGASVKVSCKASGYTFTSYDMHWVRQA |
| | Y104Q (71BJN)_Fab_HC | PGQGLEWMGRIDPQTGDIKYAQKFQGRATLTVDTSTSTAY |
| | | MELSRLRSDDTAVYYCARWDYGGQFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1447 | VL T30Y R53G | RASQDIYNYLN |
| | (71BJN)_LCDR1 | |
| 1448 | VL T30Y R53G | YTSGLHS |
| | (71BJN)_LCDR2 | |
| 1449 | VL T30Y R53G | QQGDTLPWT |
| | (71BJN)_LCDR3 | |
| 1450 | VL T30Y R53G | DIQMTQSPSSLSASVGDRVTITCRASQDIYNYLNWYQQKP |
| | (71BJN)_VL | GKAVKLLIYYTSGLHSGVPSRESGSGSGTDYTLTISSLQP |
| | | EDFATYFCQQGDTLPWTFGQGTKLEIK |
| 1451 | VL T30Y R53G | DIQMTQSPSSLSASVGDRVTITCRASQDIYNYLNWYQQKP |
| | (71BJN)_LC | GKAVKLLIYYTSGLHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1452 | VH S31Q W33D W99Y | QYDMH |
| | (72BJN)_HCDR1 | |
| 1453 | VH S31Q W33D W99Y | RIDPQSGGIKYAQKFQG |
| | (72BJN)_HCDR2 | |
| 1454 | VH S31Q W33D W99Y | YDYGGYFDV |
| | (72BJN)_HCDR3 | |
| 1455 | VH S31Q W33D W99Y | EVQLVQSGAEVKKPGASVKVSCKASGYTFTQYDMHWVRQA |
| | (72BJN)_VH | PGQGLEWMGRIDPQSGGIKYAQKFQGRATLTVDTSTSTAY |
| | | MELSRLRSDDTAVYYCARYDYGGYFDVWGQGTLVTVSS |
| 1456 | VH S31Q W33D W99Y | EVOLVQSGAEVKKPGASVKVSCKASGYTFTQYDMHWVRQA |
| | (72BJN)_Fab_HC | PGQGLEWMGRIDPQSGGIKYAQKFQGRATLTVDTSTSTAY |
| | | MELSRLRSDDTAVYYCARYDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1457 | VL T30Y R53I | RASQDIYNYLN |
| | (72BJN)_LCDR1 | |
| 1458 | VL T30Y R53I | YTSILHS |
| | (72BJN)_LCDR2 | |
| 1459 | VL T30Y R53I | QQGDTLPWT |
| | (72BJN)_LCDR3 | |
| 1460 | VL T30Y R53I | DIQMTQSPSSLSASVGDRVTITCRASQDIYNYLNWYQQKP |
| | (72BJN)_VL | GKAVKLLIYYTSILHSGVPSRFSGSGSGTDYTLTISSLQP |
| | | EDFATYFCQQGDTLPWTFGQGTKLEIK |
| 1461 | VL T30Y R53I | DIQMTQSPSSLSASVGDRVTITCRASQDIYNYLNWYQQKP |
| | (72BJN)_LC | GKAVKLLIYYTSILHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 1462 | VH W33D | SYDMH |
| | (73BJN)_HCDR1 | |
| 1463 | VH W33D | RIDPQSGGIKYAQKFQG |
| | (73BJN)_HCDR2 | |
| 1464 | VH W33D | WDYGGYFDV |
| | (73BJN)_HCDR3 | |
| 1465 | VH W33D (73BJN)_VH | EVOLVQSGAEVKKPGASVKVSCKASGYTFTSYDMHWVRQA |
| | | PGQGLEWMGRIDPQSGGIKYAQKFQGRATLTVDTSTSTAY |
| | | MELSRLRSDDTAVYYCARWDYGGYFDVWGQGTLVTVSS |
| 1466 | VH W33D | EVQLVQSGAEVKKPGASVKVSCKASGYTFTSYDMHWVRQA |
| | (73BJN)_Fab_HC | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1467 | VL T30Y | RASQDIYNYLN |
| | (73BJN)_LCDR1 | |
| 1468 | VL T30Y | YTSRLHS |
| | (73BJN)_LCDR2 | |
| 1469 | VL T30Y | QQGDTLPWT |
| | (73BJN)_LCDR3 | |
| 1470 | VL T30Y (73BJN)_VL | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIK |
| 1471 | VL T30Y (73BJN)_LC | DIQMTQSPSSLSASVGDRVTITCRASQDIYNYLNWYQQKP |
| | | GKAVKLLIYYTSRLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1472 | VH S31Y W33D M34L | YYDLH |
| | (74BJN)_HCDR1 | |
| 1473 | VH S31Y W33D M34L | RIDPQSGGIKYAQKFQG |
| | (74BJN)_HCDR2 | |
| 1474 | VH S31Y W33D M34L | WDYGGYFDV |
| | (74BJN)_HCDR3 | |
| 1475 | VH S31Y W33D M34L | EVQLVQSGAEVKKPGASVKVSCKASGYTFTYYDLHWVRQA |
| | (74BJN)_VH | PGQGLEWMGRIDPQSGGIKYAQKFQGRATLTVDTSTSTAY |
| | | MELSRLRSDDTAVYYCARWDYGGYFDVWGQGTLVTVSS |
| 1476 | VH S31Y W33D M34L | EVQLVQSGAEVKKPGASVKVSCKASGYTFTYYDLHWVRQA |
| | (74BJN)_Fab_HC | PGQGLEWMGRIDPQSGGIKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1477 | VL T30Y T93D L94I | RASQDIYNYLNW |
| | (74BJN)_LCDR1 | |
| 1478 | VL T30Y T93D L94I | YTSRLHS |
| | (74BJN)_LCDR2 | |
| 1479 | VL T30Y T93D L94I | QQGDDIPWTF |
| | (74BJN)_LCDR3 | |
| 1480 | VL T30Y T93D L94I | DIQMTQSPSSLSASVGDRVTITCRASQDIYNYLNWYQQKP |
| | (74BJN)_VL | GKAVKLLIYYTSRLHSGVPSRFSGSGSGTDYTLTISSLQP |
| | | EDFATYFCQQGDDIPWTFGQGTKLEIK |
| 1481 | VL T30Y T93D L94I | DIQMTQSPSSLSASVGDRVTITCRASQDIYNYLNWYQQKP |
| | (74BJN)_LC | GKAVKLLIYYTSRLHSGVPSRFSGSGSGTDYTLTISSLQP |
| | | EDFATYFCQQGDDIPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1482 | VH S31K W33D S55D | KYDMH |
| | I58F (75BJN)_HCDR1 | |
| 1483 | VH S31K W33D S55D | RIDPQDGGFKYAQKFQG |
| | I58F (75BJN)_HCDR2 | |
| 1484 | VH S31K W33D S55D | WDYGGYFDV |
| | I58F (75BJN)_HCDR3 | |
| 1485 | VH S31K W33D S55D | EVQLVQSGAEVKKPGASVKVSCKASGYTFTKYDMHWVRQA |
| | I58F (75BJN)_VH | PGQGLEWMGRIDPQDGGFKYAQKFQGRATLTVDTSTSTAY |
| | | MELSRLRSDDTAVYYCARWDYGGYFDVWGQGTLVTVSS |
| 1486 | VH S31K W33D S55D | EVOLVQSGAEVKKPGASVKVSCKASGYTFTKYDMHWVRQA |
| | I58F (75BJN)_Fab_HC | PGQGLEWMGRIDPQDGGFKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1487 | VL T30Y R53Y T93E | RASQDIFNYLN |
| | (75BJN)_LCDR1 | |
| 1488 | VL T30Y R53Y T93E | YTSYLHS |
| | (75BJN)_LCDR2 | |
| 1489 | VL T30Y R53Y T93E | QQGDELPWT |
| | (75BJN)_LCDR3 | |
| 1490 | VL T30Y R53Y T93E | DIQMTQSPSSLSASVGDRVTITCRASQDIFNYLNWYQQKP |
| | (75BJN)_VL | GKAVKLLIYYTSYLHSGVPSRESGSGSGTDYTLTISSLQP |
| | | EDFATYFCQQGDELPWTFGQGTKLEIK |
| 1491 | VL T30Y R53Y T93E | DIQMTQSPSSLSASVGDRVTITCRASQDIFNYLNWYQQKP |
| | (75BJN)_LC | GKAVKLLIYYTSYLHSGVPSRFSGSGSGTDYTLTISSLQP |
| | | EDFATYFCQQGDELPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1492 | VH S31Y W33D Q54Y | YYDMH |
| | S55D G57D | |
| | (76BJN)_HCDR1 | |
| 1493 | VH S31Y W33D Q54Y | RIDPYDGDIKYAQKFQG |
| | S55D G57D | |
| | (76BJN) | |
| 1494 | VH S31Y W33D Q54Y | WDYGGYFDV |
| | S55D G57D | |
| | (76BJN)_HCDR3 | |
| 1495 | VH S31Y W33D Q54Y | EVQLVQSGAEVKKPGASVKVSCKASGYTFTYYDMHWVRQA |
| | S55D G57D (76BJN)_VH | PGQGLEWMGRIDPYDGDIKYAQKFQGRATLTVDTSTSTAY |
| | | MELSRLRSDDTAVYYCARWDYGGYFDVWGQGTLVTVSS |
| 1496 | VH S31Y W33D Q54Y | EVQLVQSGAEVKKPGASVKVSCKASGYTFTYYDMHWVRQA |
| | S55D G57D | PGQGLEWMGRIDPYDGDIKYAQKFQGRATLTVDTSTSTAY |
| | (76BJN)_Fab_HC | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1497 | VL T30Y R53L T93E | RASQDIYNYLN |
| | (76BJN)_LCDR1 | |
| 1498 | VL T30Y R53L T93E | YTSLLHS |
| | (76BJN)_LCDR2 | |
| 1499 | VL T30Y R53L T93E | QQGDELPWT |
| | (76BJN)_LCDR3 | |
| 1500 | VL T30Y R53L T93E | DIQMTQSPSSLSASVGDRVTITCRASQDIYNYLNWYQQKP |
| | (76BJN)_VL | GKAVKLLIYYTSLLHSGVPSRESGSGSGTDYTLTISSLQP |
| | | EDFATYFCQQGDELPWTFGQGTKLEIK |
| 1501 | VL T30Y R53L T93E | DIQMTQSPSSLSASVGDRVTITCRASQDIYNYLNWYQQKP |
| | (76BJN)_LC | GKAVKLLIYYTSLLHSGVPSRFSGSGSGTDYTLTISSLQP |
| | | EDFATYFCQQGDELPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 1502 | VH W33D I58V | SYDMH |
| | (77BJN)_HCDR1 | |
| 1503 | VH W33D I58V | RIDPQSGGVKYAQKFQG |
| | (77BJN)_HCDR2 | |
| 1504 | VH W33D I58V | WDYGGYFDV |
| | (77BJN)_HCDR3 | |
| 1505 | VH W33D I58V | EVQLVQSGAEVKKPGASVKVSCKASGYTFTSYDMHWVRQA |
| | (77BJN)_VH | PGQGLEWMGRIDPQSGGVKYAQKFQGRATLTVDTSTSTAY |
| | | MELSRLRSDDTAVYYCARWDYGGYFDVWGQGTLVTVSS |
| 1506 | VH W33D I58V | EVQLVQSGAEVKKPGASVKVSCKASGYTFTSYDMHWVRQA |
| | (77BJN)_Fab_HC | PGQGLEWMGRIDPQSGGVKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1507 | VL T30Y R53V T93E | RASQDIYNYLN |
| | (77BJN)_LCDR1 | |
| 1508 | VL T30Y R53V T93E | YTSVLHS |
| | (77BJN)_LCDR2 | |
| 1509 | VL T30Y R53V T93E | QQGDELPWTF |
| | (77BJN)_LCDR3 | |
| 1510 | VL T30Y R53V T93E | DIQMTQSPSSLSASVGDRVTITCRASQDIYNYLNWYQQKP |
| | (77BJN)_VL | GKAVKLLIYYTSVLHSGVPSRFSGSGSGTDYTLTISSLQP |
| | | EDFATYFCQQGDELPWTFGQGTKLEIK |
| 1511 | VL T30Y R53V T93E | DIQMTQSPSSLSASVGDRVTITCRASQDIYNYLNWYQQKP |
| | (77BJN)_LC | GKAVKLLIYYTSVLHSGVPSRFSGSGSGTDYTLTISSLQP |
| | | EDFATYFCQQGDELPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 1512 | VH W33D K59R | SYDMH |
| | (78BJN)_HCDR1 | |
| 1513 | VH W33D K59R | RIDPQSGGIRYAQKFQG |
| | (78BJN)_HCDR2 | |
| 1514 | VH W33D K59R | WDYGGYFDV |
| | (78BJN)_HCDR3 | |
| 1515 | VH W33D K59R | EVOLVQSGAEVKKPGASVKVSCKASGYTFTSYDMHWVRQA |
| | (78BJN)_VH | PGQGLEWMGRIDPQSGGIRYAQKFQGRATLTVDTSTSTAY |
| | | MELSRLRSDDTAVYYCARWDYGGYFDVWGQGTLVTVSS |
| 1516 | VH W33D K59R | EVQLVQSGAEVKKPGASVKVSCKASGYTFTSYDMHWVRQA |
| | (78BJN)_Fab_HC | PGQGLEWMGRIDPQSGGIRYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1517 | VL T30Y R53N T93E | RASQDIYNYLN |
| | (78BJN)_LCDR1 | |
| 1518 | VL T30Y R53N T93E | YTSNLHS |
| | (78BJN)_LCDR2 | |
| 1519 | VL T30Y R53N T93E | QQGDELPWT |
| | (78BJN)_LCDR3 | |
| 1520 | VL T30Y R53N T93E | DIQMTQSPSSLSASVGDRVTITCRASQDIYNYLNWYQQKP |
| | (78BJN)_VL | GKAVKLLIYYTSNLHSGVPSRFSGSGSGTDYTLTISSLQP |
| | | EDFATYFCQQGDELPWTFGQGTKLEIK |
| 1521 | VL T30Y R53N T93E | DIQMTQSPSSLSASVGDRVTITCRASQDIYNYLNWYQQKP |
| | (78BJN)_LC | GKAVKLLIYYTSNLHSGVPSRFSGSGSGTDYTLTISSLQP |
| | | EDFATYFCQQGDELPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 1522 | VH W33D S55D S57D | SYDMH |
| | I58V (79BJN)_HCDR1 | |
| 1523 | VH W33D S55D S57D | RIDPQDGDVKYAQKFQG |
| | I58V (79BJN)_HCDR2 | |
| 1524 | VH W33D S55D S57D | WDYGGYFDV |
| | I58V (79BJN HCDR3 | |
| 1525 | VH W33D S55D S57D | EVQLVQSGAEVKKPGASVKVSCKASGYTFTSYDMHWVRQA |
| | I58V (79BJN)_VH | PGQGLEWMGRIDPQDGDVKYAQKFQGRATLTVDTSTSTAY |
| | | MELSRLRSDDTAVYYCARWDYGGYFDVWGQGTLVTVSS |
| 1526 | VH W33D S55D S57D | EVQLVQSGAEVKKPGASVKVSCKASGYTFTSYDMHWVRQA |
| | I58V (79BJN)_Fab_HC | PGQGLEWMGRIDPQDGDVKYAQKFQGRATLTVDTSTSTAY |
| | | MELSRLRSDDTAVYYCARWDYGGYFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1527 | VL T30Y R53G T93E | RASQDIYNYLN |
| | (79BJN)_LCDR1 | |
| 1528 | VL T30Y R53G T93E | YTSGLHS |
| | (79BJN)_LCDR2 | |
| 1529 | VL T30Y R53G T93E | QQGDELPWT |
| | (79BJN)_LCDR3 | |
| 1530 | VL T30Y R53G T93E | DIQMTQSPSSLSASVGDRVTITCRASQDIYNYLNWYQQKP |
| | (79BJN)_VL | GKAVKLLIYYTSGLHSGVPSRFSGSGSGTDYTLTISSLQP |
| | | EDFATYFCQQGDELPWTFGQGTKLEIK |
| 1531 | VL T30Y R53G T93E | DIQMTQSPSSLSASVGDRVTITCRASQDIYNYLNWYQQKP |
| | (79BJN)_LC | GKAVKLLIYYTSGLHSGVPSRFSGSGSGTDYTLTISSLQP |
| | | EDFATYFCQQGDELPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1532 | VH W33D K59R Y104Q | SYDMH |
| | (80BJN)_HCDR1 | |
| 1533 | VH W33D K59R Y104Q | RIDPQSGGIRYAQKFQG |
| | (80BJN)_HCDR2 | |
| 1534 | VH W33D K59R Y104Q | WDYGGQFDV |
| | (80BJN)_HCDR3 | |
| 1535 | VH W33D K59R Y104Q | EVQLVQSGAEVKKPGASVKVSCKASGYTFTSYDMHWVRQA |
| | (80BJN)_VH | PGQGLEWMGRIDPQSGGIRYAQKFQGRATLTVDTSTSTAY |
| | | MELSRLRSDDTAVYYCARWDYGGQFDVWGQGTLVTVSS |
| 1536 | VH W33D K59R Y104Q | EVOLVQSGAEVKKPGASVKVSCKASGYTFTSYDMHWVRQA |
| | (80BJN)_Fab_HC | PGQGLEWMGRIDPQSGGIRYAQKFQGRATLTVDTSTSTAY |
| | | MELSRLRSDDTAVYYCARWDYGGQFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1537 | VL T30Y R53I | RASQDIYNYLN |
| | (80BJN)_LCDR1 | |
| 1538 | VL T30Y R53I | YTSILHS |
| | (80BJN)_LCDR2 | |
| 1539 | VL T30Y R53I | QQGDTLPWT |
| | (80BJN)_LCDR3 | |
| 1540 | VL T30Y R53I | DIQMTQSPSSLSASVGDRVTITCRASQDIYNYLNWYQQKP |
| | (80BJN)_VL | GKAVKLLIYYTSILHSGVPSRESGSGSGTDYTLTISSLQP |
| | | EDFATYFCQQGDTLPWTFGQGTKLEIK |
| 1541 | VL T30Y R53I | DIQMTQSPSSLSASVGDRVTITCRASQDIYNYLNWYQQKP |
| | (80BJN)_LC | GKAVKLLIYYTSILHSGVPSRESGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 1542 | VH S31E W33D K59R | EYDMH |
| | Y104Q V107A | |
| | (81BJN)_HCDR1 | |
| 1543 | VH S31E W33D K59R | RIDPQSGGIRYAQKFQG |
| | Y104Q V107A | |
| | (81BJN)_HCDR2 | |
| 1544 | VH S31E W33D K59R | WDYGGQFDA |
| | Y104Q V107A | |
| | (81BJN)_HCDR3 | |
| 1545 | VH S31E W33D K59R | EVOLVQSGAEVKKPGASVKVSCKASGYTFTEYDMHWVRQA |
| | Y104Q V107A | PGQGLEWMGRIDPQSGGIRYAQKFQGRATLTVDTSTSTAY |
| | (81BJN)_VH | MELSRLRSDDTAVYYCARWDYGGQFDAWGQGTLVTVSS |
| 1546 | VH S31E W33D K59R | EVQLVQSGAEVKKPGASVKVSCKASGYTFTEYDMHWVRQA |
| | Y104Q V107A | PGQGLEWMGRIDPQSGGIRYAQKFQGRATLTVDTSTSTAY |
| | (81BJN)_Fab_HC | MELSRLRSDDTAVYYCARWDYGGQFDAWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1547 | VL T30Y R53V | RASQDIYNYLN |
| | (81BJN)_LCDR1 | |
| 1548 | VL T30Y R53V | YTSVLHS |
| | (81BJN)_LCDR2 | |
| 1549 | VL T30Y R53V | QQGDTLPWT |
| | (81BJN)_LCDR3 | |
| 1550 | VL T30Y R53V | DIQMTQSPSSLSASVGDRVTITCRASQDIYNYLNWYQQKP |
| | (81BJN)_VL | GKAVKLLIYYTSVLHSGVPSRESGSGSGTDYTLTISSLQP |
| | | EDFATYFCQQGDTLPWTFGQGTKLEIK |
| 1551 | VL T30Y R53V | DIQMTQSPSSLSASVGDRVTITCRASQDIYNYLNWYQQKP |
| | (81BJN)_LC | GKAVKLLIYYTSVLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 1552 | VH W33Y H35N 158V | SYYMN |
| | D100H Y104Q | |
| | (82BJN)_HCDR1 | |
| 1553 | VH W33Y H35N I58V | RIDPQSGGVKYAQKFQG |
| | D100H Y104Q | |
| | (82BJN)_HCDR2 | |
| 1554 | VH W33Y H35N I58V | WHYGGQFDV |
| | D100H Y104Q | |
| | (82BJN)_HCDR3 | |
| 1555 | VH W33Y H35N I58V | EVQLVQSGAEVKKPGASVKVSCKASGYTFTSYYMNWVRQA |
| | D100H Y104Q | PGQGLEWMGRIDPQSGGVKYAQKFQGRATLTVDTSTSTAY |
| | (82BJN)_VH | MELSRLRSDDTAVYYCARWHYGGQFDVWGQGTLVTVSS |
| 1556 | VH W33Y H35N I58V | EVQLVQSGAEVKKPGASVKVSCKASGYTFTSYYMNWVRQA |
| | D100H Y104Q | PGQGLEWMGRIDPQSGGVKYAQKFQGRATLTVDTSTSTAY |
| | (82BJN)_Fab_HC | MELSRLRSDDTAVYYCARWHYGGQFDVWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHT |
| 1557 | VL T30Y R53G | RASQDIYNYLN |
| | (82BJN)_LCDR1 | |
| 1558 | VL T30Y R53G | YTSGLHS |
| | (82BJN)_LCDR2 | |
| 1559 | VL T30Y R53G | QQGDTLPWT |
| | (82BJN)_LCDR3 | |
| 1560 | VL T30Y R53G | DIQMTQSPSSLSASVGDRVTITCRASQDIYNYLNWYQQKP |
| | (82BJN)_VL | GKAVKLLIYYTSGLHSGVPSRESGSGSGTDYTLTISSLQP |
| | | EDFATYFCQQGDTLPWTFGQGTKLEIK |
| 1561 | VL T30Y R53G | DIQMTQSPSSLSASVGDRVTITCRASQDIYNYLNWYQQKP |
| | (82BJN)_LC | GKAVKLLIYYTSGLHSGVPSRFSGSGSGTDYTLTISSLQP |
| | | EDFATYFCQQGDTLPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |

TABLE 43
CDR sequences of 20E6 variants isolated from yeast display selection

| 55BJN | Kabat numbering scheme | | |
|---|---|---|---|
| V-region ID | CDR1 | CDR2 | CDR3 |
| VH W33D M34I | SYDIH | RIDPQSGGIKYAQKFQG | WDYGGYFDV |
| (55BJN) | (SEQ ID NO: | (SEQ ID NO: 1566) | (SEQ ID NO: |
| | 1565) | | 1567) |
| VL (55BJN) | RASQDITNYLN | YTSRLHS | QQGDTLPWT |
| | (SEQ ID NO: | (SEQ ID NO: 1569) | (SEQ ID NO: |
| | 1568) | | 1570) |
| | Chothia numbering scheme | | |
| | CDR1 | CDR2 | CDR3 |
| VH W33D M34I | GYTFTSY | DPQSGG | WDYGGYFDV |
| (55BJN) | (SEQ ID NO: | (SEQ ID NO: 1572) | (SEQ ID NO: |
| | 1571) | | 1573) |
| VL (55BJN) | RASQDITNYLN | YTSRLHS | QQGDTLPWT |
| | (SEQ ID NO: | (SEQ ID NO: 1575) | (SEQ ID NO: |
| | 1574) | | 1576) |
| | ABM numbering scheme | | |
| | CDR1 | CDR2 | CDR3 |
| VH W33D M34I | GYTFTSYDIH | RIDPQSGGIK | WDYGGYFDV |
| (55BJN) | (SEQ ID NO: | (SEQ ID NO: 1578) | (SEQ ID NO: |
| | 1577) | | 1579) |
| VL (55BJN) | RASQDITNYLN | YTSRLHS | QQGDTLPWT |
| | (SEQ ID NO: | (SEQ ID NO: 1581) | (SEQ ID NO: |
| | 1580) | | 1582) |
| | IMGT numbering scheme | | |
| | CDR1 | CDR2 | CDR3 |
| VH W33D M34I | GYTFTSYD | IDPQSGGI | ARWDYGGYFDV |
| (55BJN) | (SEQ ID NO: | (SEQ ID NO: 1584) | (SEQ ID NO: |
| | 1583) | | 1585) |
| VL (55BJN) | QDITNY | YTS | QQGDTLPWT |
| | (SEQ ID NO: | (SEQ ID NO: 1587) | (SEQ ID NO: |
| | 1586) | | 1588) |
| 56BJN | Kabat numbering scheme | | |
| V-region ID | CDR1 | CDR2 | CDR3 |
| VH W33D M34I | SYDIH | RIDPYSGGIKYAQKFQG | WDYGGYFDV |
| Q54Y (56BJN) | (SEQ ID NO: | (SEQ ID NO: 1590) | (SEQ ID NO: |
| | 1589) | | 1591) |
| VL (56BJN)_VL | RASQDITNYLN | YTSRLHS | QQGDTLPWT |
| | (SEQ ID NO: | (SEQ ID NO: 1593) | (SEQ ID NO: |
| | 1592) | | 1594) |
| | Chothia numbering scheme | | |
| | CDR1 | CDR2 | CDR3 |
| VH W33D M34I | GYTFTSY | DPYSGG | WDYGGYFDV |
| Q54Y (56BJN) | (SEQ ID NO: | (SEQ ID NO: 1596) | (SEQ ID NO: |
| | 1595) | | 1597) |
| VL (56BJN)_VL | RASQDITNYLN | YTSRLHS | QQGDTLPWT |
| | (SEQ ID NO: | (SEQ ID NO: 1599) | (SEQ ID NO: |
| | 1598) | | 1600) |
| | ABM numbering scheme | | |
| | CDR1 | CDR2 | CDR3 |
| VH W33D M34I | GYTFTSYDIH | RIDPYSGGIK | WDYGGYFDV |
| Q54Y (56BJN) | (SEQ ID NO: | (SEQ ID NO: 1602) | (SEQ ID NO: |
| | 1601) | | 1603) |
| VL (56BJN)_VL | RASQDITNYLN | YTSRLHS | QQGDTLPWT |
| | (SEQ ID NO: | (SEQ ID NO: 1605) | (SEQ ID NO: |
| | 1604) | | 1606) |
| | IMGT numbering scheme | | |
| | CDR1 | CDR2 | CDR3 |
| VH W33D M34I | GYTFTSYD | IDPYSGGI | ARWDYGGYFDV |
| Q54Y (56BJN) | (SEQ ID NO: | (SEQ ID NO: | (SEQ ID NO: |
| | 1607) | 1608) | 1609) |
| VL (56BJN)_VL | QDITNY | YTS | QQGDTLPWT |
| | (SEQ ID NO: | (SEQ ID NO: 1611) | (SEQ ID NO: |
| | 1610) | | 1612) |
| 57BJN | Kabat numbering scheme | | |
| V-region ID | CDR1 | CDR2 | CDR3 |
| VH W33D | SYDMH | RIDPQSGGIKYAQKFQG | WDYGGYFDV |
| (57BJN) | (SEQ ID NO: | (SEQ ID NO: 1614) | (SEQ ID NO: |
| | 1613) | | 1615) |
| VL (57BJN) | RASQDITNYLN | YTSRLHS | QQGDTLPWT |
| | (SEQ ID NO: | (SEQ ID NO: 1617) | (SEQ ID NO: |
| | 1616) | | 1618) |
| | Chothia numbering scheme | | |
| | CDR1 | CDR2 | CDR3 |
| VH W33D | GYTFTSY | DPQSGG | WDYGGYFDV |
| (57BJN) | (SEQ ID NO: | (SEQ ID NO: 1620) | (SEQ ID NO: |
| | 1619) | | 1621) |
| VL (57BJN) | RASQDITNYLN | YTSRLHS | QQGDTLPWT |
| | (SEQ ID NO: | (SEQ ID NO: 1623) | (SEQ ID NO: |
| | 1622) | | 1624) |
| | ABM numbering scheme | | |
| | CDR1 | CDR2 | CDR3 |
| VH W33D | GYTFTSYDMH | RIDPQSGGIK | WDYGGYFDV |
| (57BJN) | (SEQ ID NO: | (SEQ ID NO: 1626) | (SEQ ID NO: |
| | 1625) | | 1627) |
| VL (57BJN) | RASQDITNYLN | YTSRLHS | QQGDTLPWT |
| | (SEQ ID NO: | (SEQ ID NO: 1629) | (SEQ ID NO: |
| | 1628) | | 1630) |
| | IMGT numbering scheme | | |
| | CDR1 | CDR2 | CDR3 |
| VH W33D | GYTFTSYD | IDPQSGGI | ARWDYGGYFDV |
| (57BJN) | (SEQ ID NO: | (SEQ ID NO: 1632) | (SEQ ID NO: |
| | 1631) | | 1633) |
| VL (57BJN) | QDITNY | YTS | QQGDTLPWT |
| | (SEQ ID NO: | (SEQ ID NO: 1635) | (SEQ ID NO: |
| | 1634) | | 1636) |
| 58BJN | Kabat numbering scheme | | |
| V-region ID | CDR1 | CDR2 | CDR3 |
| VH W33D W99Y | SYDMH | RIDPQSGGIKYAQKFQG | YDHGGYFDV |
| Y101H (58BJN) | (SEQ ID NO: | (SEQ ID NO: 1638) | (SEQ ID NO: |
| | 1637) | | 1639) |
| VL T30Y | RASQDIYNYLN | YTSRLHS | QQGDTLPWT |
| (58BJN) | (SEQ ID NO: | (SEQ ID NO: 1641) | (SEQ ID NO: |
| | 1640) | | 1642) |
| | Chothia numbering scheme | | |
| | CDR1 | CDR2 | CDR3 |
| VH W33D W99Y | GYTFTSY | DPQSGG | YDHGGYFDV |
| Y101H (58BJN) | (SEQ ID NO: | (SEQ ID NO: 1644) | (SEQ ID NO: |
| | 1643) | | 1645) |
| VL T30Y | RASQDIYNYLN | YTSRLHS | QQGDTLPWT |
| (58BJN) | (SEQ ID NO: | (SEQ ID NO: 1647) | (SEQ ID NO: |
| | 1646) | | 1648) |
| | ABM numbering scheme | | |
| | CDR1 | CDR2 | CDR3 |
| VH W33D W99Y | GYTFTSYDMH | RIDPQSGGIK | YDHGGYFDV |
| Y101H (58BJN) | (SEQ ID NO: | (SEQ ID NO: 1650) | (SEQ ID NO: |
| | 1649) | | 1651) |
| VL T30Y | RASQDIYNYLN | YTSRLHS | QQGDTLPWT |
| (58BJN) | (SEQ ID NO: | (SEQ ID NO: 1653) | (SEQ ID NO: |
| | 1652) | | 1654) |
| | IMGT numbering scheme | | |
| | CDR1 | CDR2 | CDR3 |
| VH W33D W99Y | GYTFTSYD | IDPQSGGI | ARYDHGGYFDV |
| Y101H (58BJN) | (SEQ ID NO: | (SEQ ID NO: 1656) | (SEQ ID NO: |
| | 1655) | | 1657) |
| VL T30Y | QDIYNY | YTS | QQGDTLPWT |
| (58BJN) | | (SEQ ID NO: 1659) | |
| | (SEQ ID NO: | | (SEQ ID NO: |
| | 1658) | | 1660) |
| 59BJN | Kabat numbering scheme | | |
| V-region ID | CDR1 | CDR2 | CDR3 |
| VH W33Q M34V | SYQVH | RIDPQSGGIKYAQKFQG | DYGGYFDV |
| (59BJN) | (SEQ ID NO: | (SEQ ID NO: 1662) | (SEQ ID NO: |
| | 1661) | | 1663) |
| VL T30Y | RASQDIYNYLN | YTSRLHS | QQGDTLPWT |
| (59BJN) | (SEQ ID NO: | (SEQ ID NO: 1665) | (SEQ ID NO: |
| | 1664) | | 1666) |
| | Chothia numbering scheme | | |
| | CDR1 | CDR2 | CDR3 |
| VH W33Q M34V | GYTFTSY | DPQSGG | DYGGYFDV |
| (59BJN) | (SEQ ID NO: | (SEQ ID NO: 1668) | (SEQ ID NO: |
| | 1667) | | 1669) |
| VL T30Y | RASQDIYNYLN | YTSRLHS | QQGDTLPWT |
| (59BJN) | (SEQ ID NO: | (SEQ ID NO: 1671) | (SEQ ID NO: |
| | 1670) | | 1672) |
| | ABM numbering scheme | | |
| | CDR1 | CDR2 | CDR3 |
| VH W33Q M34V | GYTFTSYQVH | RIDPQSGGIK | DYGGYFDV |
| (59BJN) | (SEQ ID NO: | (SEQ ID NO: 1674) | (SEQ ID NO: |
| | 1673) | | 1675) |
| VL T30Y | RASQDIYNYLN | YTSRLHS | QQGDTLPWT |
| (59BJN) | (SEQ ID NO: | (SEQ ID NO: 1677) | (SEQ ID NO: |
| | 1676) | | 1678) |
| | IMGT numbering scheme | | |
| | CDR1 | CDR2 | CDR3 |
| VH W33Q M34V | GYTFTSYQ | IDPQSGGI | ARDYGGYFDV |
| (59BJN) | (SEQ ID NO: | (SEQ ID NO: 1680) | (SEQ ID NO: |
| | 1679) | | 1681) |
| VL T30Y | QDIYNY | YTS | QQGDTLPWT |
| (59BJN) | (SEQ ID NO: | (SEQ ID NO: 1683) | (SEQ ID NO: |
| | 1682) | | 1684) |
| 60BJN | Kabat numbering scheme | | |
| V-region ID | CDR1 | CDR2 | CDR3 |
| VH W33D I58E | SYDMH | RIDPQSGGEKYAQKFQG | YDYGGYFDV |
| W99Y (60BJN) | (SEQ ID NO: | (SEQ ID NO: 1686) | (SEQ ID NO: |
| | 1685) | | 1687) |
| VL T30Y | RASQDIYNYLN | YTSRLHS | QQGDTLPWT |
| (60BJN) | (SEQ ID NO: | (SEQ ID NO: 1689) | (SEQ ID NO: |
| | 1688) | | 1690) |
| | Chothia numbering scheme | | |
| | CDR1 | CDR2 | CDR3 |
| VH W33D I58E | GYTFTSY | DPQSGG | YDYGGYFDV |
| W99Y (60BJN) | (SEQ ID NO: | (SEQ ID NO: 1692) | (SEQ ID NO: |
| | 1691) | YTSRLHS | 1693) |
| VL T30Y | RASQDIYNYLN | (SEQ ID NO: 1695) | QQGDTLPWT |
| (60BJN) | (SEQ ID NO: | | (SEQ ID NO: |
| | 1694) | | 1696) |
| | ABM numbering scheme | | |
| | CDR1 | CDR2 | CDR3 |
| VH W33D I58E | GYTFTSYDMH | RIDPQSGGEK | YDYGGYFDV |
| W99Y (60BJN) | (SEQ ID NO: | (SEQ ID NO: 1698) | (SEQ ID NO: |
| | 1697) | | 1699) |
| VL T30Y | RASQDIYNYLN | YTSRLHS | QQGDTLPWT |
| (60BJN) | (SEQ ID NO: | (SEQ ID NO: 1701) | (SEQ ID NO: |
| | 1700) | | 1702) |
| | IMGT numbering scheme | | |
| | CDR1 | CDR2 | CDR3 |
| VH W33D I58E | GYTFTSYD | IDPQSGGE | ARYDYGGYFDV |
| W99Y (60BJN) | (SEQ ID NO: | (SEQ ID NO: 1704) | (SEQ ID NO: |
| | 1703) | | 1705) |
| VL T30Y | QDIYNY | YTS | QQGDTLPWT |
| (60BJN) | (SEQ ID NO: | (SEQ ID NO: 1707) | (SEQ ID NO: |
| | 1706) | | 1708) |
| 61BJN | Kabat numbering scheme | | |
| V-region ID | CDR1 | CDR2 | CDR3 |
| VH S31D W33D | DYDMH | RIDPQSGDHDYAQKFQG | YDHGGYFDV |
| G57D I58H | (SEQ ID NO: | (SEQ ID NO: 1710) | (SEQ ID NO: |
| K59D W99Y | 1709) | | 1711) |
| Y101H (61BJN) | | | |
| VL T30Y | RASQDIYNYLN | YTSRLHS | QQGDTLPWT |
| (61BJN) | (SEQ ID NO: | (SEQ ID NO: 1713) | (SEQ ID NO: |
| | 1712) | | 1714) |
| | Chothia numbering scheme | | |
| | CDR1 | CDR2 | CDR3 |
| VH S31D W33D | GYTFTDY | DPQSGD | GYTFTDY |
| G57D I58H | (SEQ ID NO: | (SEQ ID NO: 1716) | (SEQ ID NO: |
| K59D W99Y | 1715) | | 1715) |
| Y101H (61BJN) | | | |
| VL T30Y | RASQDIYNYLN | YTSRLHS | QQGDTLPWT |
| (61BJN) | (SEQ ID NO: | (SEQ ID NO: 1719) | (SEQ ID NO: |
| | 1718) | | 1720) |
| | ABM numbering scheme | | |
| | CDR1 | CDR2 | CDR3 |
| VH S31D W33D | GYTFTDYDMH | RIDPQSGDHD | YDHGGYFDV |
| G57D I58H | (SEQ ID NO: | (SEQ ID NO: 1722) | (SEQ ID NO: |
| | 1721) | | 1723) |
| VL T30Y | RASQDIYNYLN | YTSRLHS | QQGDTLPWT |
| (61BJN) | (SEQ ID NO: | (SEQ ID NO: 1725) | (SEQ ID NO: |
| K59D W99Y | 1724) | | 1726) |
| Y101H (61BJN) | | | |
| | IMGT numbering scheme | | |
| | CDR1 | CDR2 | CDR3 |
| VH S31D W33D | GYTFTDYD | IDPQSGDH | ARYDHGGYFDV |
| G57D I58H | (SEQ ID NO: | (SEQ ID NO: 1728) | (SEQ ID NO: |
| K59D W99Y | 1727) | | 1729) |
| Y101H (61BJN) | | | |
| VL T30Y | QDIYNY | YTS | QQGDTLPWT |
| (61BJN) | (SEQ ID NO: | (SEQ ID NO: 1731) | (SEQ ID NO: |
| | 1730) | | 1732) |
| 62BJN | Kabat numbering scheme | | |
| V-region ID | CDR1 | CDR2 | CDR3 |
| VH W33D Q54Y | SYDMH | RIDPYGGGIKYAQKFQG | YDHGGYFDV |
| S55G W99Y | (SEQ ID NO: | (SEQ ID NO: 1734) | (SEQ ID NO: |
| Y101H (62BJN) | 1733) | | 1735) |
| VL T30Y | RASQDIYNYLN | YTSRLHS | QQGDTLPWT |
| (62BJN) | (SEQ ID NO: | (SEQ ID NO: 1737) | (SEQ ID NO: |
| | 1736) | | 1738) |
| | Chothia numbering scheme | | |
| | CDR1 | CDR2 | CDR3 |
| VH W33D Q54Y | GYTFTSY | DPYGGG | YDHGGYFDV |
| S55G W99Y | (SEQ ID NO: | (SEQ ID NO: 1740) | (SEQ ID NO: |
| Y101H (62BJN) | 1739) | | 1741) |
| VL T30Y | RASQDIYNYLN | YTSRLHS | QQGDTLPWT |
| (62BJN) | (SEQ ID NO: | (SEQ ID NO: 1743) | (SEQ ID NO: |
| | 1742) | | 1744) |
| | ABM numbering scheme | | |
| | CDR1 | CDR2 | CDR3 |
| VH W33D Q54Y | GYTFTSYDMH | RIDPYGGGIK | YDHGGYFDV |
| S55G W99Y | (SEQ ID NO: | (SEQ ID NO: 1746) | (SEQ ID NO: |
| Y101H (62BJN) | 1745) | | 1747) |
| VL T30Y | RASQDIYNYLN | YTSRLHS | QQGDTLPWT |
| (62BJN) | (SEQ ID NO: | (SEQ ID NO: 1749) | (SEQ ID NO: |
| | 1748) | | 1750) |
| | IMGT numbering scheme | | |
| | CDR1 | CDR2 | CDR3 |
| VH W33D Q54Y | GYTFTSYD | IDPYGGGI | ARYDHGGYFDV |
| S55G W99Y | (SEQ ID NO: | (SEQ ID NO: 1752) | (SEQ ID NO: |
| Y101H (62BJN) | 1751) | | 1753) |
| VL T30Y | QDIYNY | YTS | QQGDTLPWT |
| (62BJN) | | (SEQ ID NO: 1755) | |
| | (SEQ ID NO: | | (SEQ ID NO: |
| | 1754) | | 1756) |
| 63BJN | Kabat numbering scheme | | |
| V-region ID | CDR1 | CDR2 | CDR3 |
| VH S31F W33D | FYDIH | RIDPQSGGIKYAQKFQG | WDYGGYFDV |
| M34I (63BJN) | (SEQ ID NO: | (SEQ ID NO: 1758) | (SEQ ID NO: |
| | 1757) | | 1759) |
| VL T30Y | RASQDIYNYLN | YTSRLHS | QQGDTLPWT |
| (63BJN) | (SEQ ID NO: | (SEQ ID NO: 1761) | (SEQ ID NO: |
| | 1760) | | 1762) |
| | Chothia numbering scheme | | |
| | CDR1 | CDR2 | CDR3 (*) |
| VH S31F W33D | GYTFTFY | DPQSGG | WDYGGYFDV |
| M34I (63BJN) | (SEQ ID NO: | (SEQ ID NO: 1764) | (SEQ ID NO: |
| | 1763) | | 1765) |
| VL T30Y | RASQDIYNYLN | YTSRLHS | QQGDTLPWT |
| (63BJN) | (SEQ ID NO: | (SEQ ID NO: 1767) | (SEQ ID NO: |
| | 1766) | | 1768) |
| | ABM numbering scheme | | |
| | CDR1 | CDR2 | CDR3 |
| VH S31F W33D | GYTFTFYDIH | RIDPQSGGIK | WDYGGYFDV |
| M34I (63BJN) | (SEQ ID NO: | (SEQ ID NO: 1770) | (SEQ ID NO: |
| | 1769) | | 1771) |
| VL T30Y | RASQDIYNYLN | YTSRLHS | QQGDTLPWT |
| (63BJN) | (SEQ ID NO: | (SEQ ID NO: 1773) | (SEQ ID NO: |
| | 1772) | | 1774) |
| | IMGT numbering scheme | | |
| | CDR1 | CDR2 | CDR3 |
| VH S31F W33D | GYTFTFYD | IDPQSGGI | ARWDYGGYFDV |
| M34I (63BJN) | (SEQ ID NO: | (SEQ ID NO: 1776) | (SEQ ID NO: |
| | 1775) | | 1777) |
| VL T30Y | QDIYNY | YTS | QQGDTLPWT |
| (63BJN) | (SEQ ID NO: | (SEQ ID NO: 1779) | (SEQ ID NO: |
| | 1778) | | 1780) |
| 64BJN | Kabat numbering scheme | | |
| V-region ID | CDR1 | CDR2 | CDR3 |
| VH S31T I58S | TYWMH | RIDPQSGGSKYAQKFQG | WDYGGYFDV |
| (64BJN) | (SEQ ID NO: | (SEQ ID NO: 1782) | (SEQ ID NO: |
| | 1781) | | 1783) |
| VL T30Y R53H | RASQDIYNYLN | YTSHLHS | QQGDTLPWT |
| (64BJN) | (SEQ ID NO: | (SEQ ID NO: 1785) | (SEQ ID NO: |
| | 1784) | | 1786) |
| | Chothia numbering scheme | | |
| | CDR1 | CDR2 | CDR3 |
| VH S31T I58S | GYTFTTY | DPQSGG | WDYGGYFDV |
| (64BJN) | (SEQ ID NO: | (SEQ ID NO: 1788) | (SEQ ID NO: |
| | 1787) | | 1789) |
| VL T30Y R53H | RASQDIYNYLN | YTSHLHS | QQGDTLPWT |
| (64BJN) | (SEQ ID NO: | (SEQ ID NO: 1791) | (SEQ ID NO: |
| | 1790) | | 1792) |
| | ABM numbering scheme | | |
| | CDR1 | CDR2 | CDR3 |
| VH S31T I58S | GYTFTTYWMH | RIDPQSGGSK | WDYGGYFDV |
| (64BJN) | (SEQ ID NO: | (SEQ ID NO: 1794) | (SEQ ID NO: |
| | 1793) | | 1795) |
| VL T30Y R53H | RASQDIYNYLN | YTSHLHS | QQGDTLPWT |
| (64BJN) | (SEQ ID NO: | (SEQ ID NO: 1797) | (SEQ ID NO: |
| | 1796) | | 1798) |
| | IMGT numbering scheme | | |
| | CDR1 | CDR2 | CDR3 |
| VH S3IT I58S | GYTFTTYW | IDPQSGGS | AJWDYGGYFDV |
| (64BJN) | (SEQ ID NO: | (SEQ ID NO: 1800) | (SEQ ID NO: |
| | 1799) | | 1801) |
| VL T30Y R53H | QDIYNY | YTS | QQGDTLPWT |
| (64BJN) | (SEQ ID NO: | (SEQ ID NO: 1803) | (SEQ ID NO: |
| | 1802) | | 1804) |
| 65BJN | Kabat numbering scheme | | |
| V-region ID | CDR1 | CDR2 | CDR3 |
| VH W33D M34L | SYDLH | RIDPQSGGIKYAQKFQG | YDQGGYFDV |
| W99Y Y101Q | (SEQ ID NO: | (SEQ ID NO: 1806) | (SEQ ID MO: |
| (65BJN) | 1805) | | 1807) |
| VL T30Y N31Y | RASQDIYYYLN | YTSRLHS | QQGDTLPWT |
| (65BJN) | (SEQ ID NO: | (SEQ ID NO: 1809) | (SEQ ID NO: |
| | 1808) | | 1810) |
| | Chothia numbering scheme | | |
| | CDR1 | CDR2 | CDR3 |
| VH W33D M34L | GYTFTSY | DPQSGG | YDQGGYFDV |
| W99Y Y101Q | (SEQ ID NO: | (SEQ ID NO: 1812) | (SEQ ID NO: |
| (65BJN) | 1811) | | 1813) |
| VL T30Y N31Y | RASQDIYYYLN | YTSRLHS | QQGDTLPWT |
| (65BJN) | (SEQ ID NO: | (SEQ ID NO: 1815) | (SEQ ID NO: |
| | 1814) | | 1816) |
| | ABM numbering scheme | | |
| | CDR1 | CDR2 | CDR3 |
| VH W33D M34L | GYTFTSYDLH | RIDPQSGGIK | YDQGGYFDV |
| W99Y Y101Q | (SEQ ID NO: | (SEQ ID NO: 1818) | (SEQ ID NO: |
| (65BJN) | 1817) | | 1819) |
| VL T30Y N31Y | RASQDIYYYLN | YTSRLHS | QQGDTLPWT |
| (65BJN) | (SEQ ID NO: | (SEQ ID NO: 1821) | (SEQ ID NO: |
| | 1820) | | 1822) |
| | IMGT numbering scheme | | |
| | CDR1 | CDR2 | CDR3 |
| VH W33D M34L | GYTFTSYD | IDPQSGGI | ARYDQGGYFDV |
| W99Y Y101Q | (SEQ ID NO: | (SEQ ID NO: 1824) | (SEQ ID NO: |
| (65BJN) | 1823) | | 1825) |
| VL T30Y N31Y | QDIYYY | YTS | QQGDTLPWT |
| (65BJN) | (SEQ ID NO: | (SEQ ID NO: 1827) | (SEQ ID NO: |
| | 1826) | | 1828) |
| 66BJN | Kabat numbering scheme | | |
| V-region ID | CDR1 | CDR2 | CDR3 |
| VH W33D S55T | SYDMH | RIDPQTGGIKYAQKFQG | WDHGGYFDV |
| Y101H (66BJN) | (SEQ ID NO: | (SEQ ID NO: 1830) | (SEQ ID NO: |
| | 1829) | | 1831) |
| VL T30Y L94Q | RASQDIYNYLN | YTSRLHS | QQGDTQPWT |
| (66BJN) | (SEQ ID NO: | (SEQ ID NO: 1833) | (SEQ ID NO: |
| | 1832) | | 1834) |
| | Chothia numbering scheme | | |
| | CDR1 | CDR2 | CDR3 |
| VH VJ33D S55T | GYTFTSY | DPQTGG | WDHGGYFDV |
| Y101H (66BJN) | (SEQ ID NO: | (SEQ ID NO: 1836) | (SEQ ID NO: |
| | 1835) | | 1837) |
| VL T30Y L94Q | RASQDIYNYLN | YTSRLHS | QQGDTQPWT |
| (66BJN) | (SEQ ID NO: | (SEQ ID NO: 1839) | (SEQ ID NO: |
| | 1838) | | 1840) |
| | ABM numbering scheme | | |
| | CDR1 | CDR2 | CDR3 |
| VH W33D S55T | GYTFTSYDMH | RIDPQTGGIK | WDHGGYFDV |
| Y101H {66BJN) | (SEQ ID NO: | (SEQ ID NO: 1842) | (SEQ ID NO: |
| | 1841) | | 1843) |
| VL T30Y L94Q | RASQDIYNYLN | YTSRLHS | QQGDTQPWT |
| (66BJN) | (SEQ ID NO: | (SEQ ID NO: 1845) | (SEQ ID NO: |
| | 1844) | | 1846) |
| | IMGT numbering scheme | | |
| | CDR1 | CDR2 | CDR3 |
| VH W33D S55T | GYTFTSYD | IDPQTGGI | ARWDHGGYFDV |
| Y101H (66BJN) | (SEQ ID NO: | (SEQ ID NO: 1848) | (SEQ ID NO: |
| | 1847) | | 1849) |
| VL T30Y L94Q | QDIYNY | YTS | QQGDTQPWT |
| (66BJN) | (SEQ ID NO: | (SEQ ID NO: 1851) | (SEQ ID NO: |
| | 1850) | | 1852) |
| 67BJN | Kabat numbering scheme | | |
| V-region ID | CDR1 | CDR2 | CDR3 |
| VH W33D | SYDMH | RIDPQSGGIKYAQKFQG | WDYGGYFDV |
| (67BJN) | (SEQ ID NO: | (SEQ ID NO: 1854) | (SEQ ID NO: |
| | 1853) | | 1855) |
| VL T30Y R53V | RASQDIYNYLN | YTSVLHS | QQGDTLPWT |
| (67BJN) | (SEQ ID NO: | (SEQ ID NO: 1857) | (SEQ ID NO: |
| | 1856) | | 1858) |
| | Chothia numbering scheme | | |
| | CDR1 | CDR2 | CDR3 |
| VH W33D | GYTFTSY | DPQSGG | WDYGGYFDV |
| (67BJN) | (SEQ ID NO: | (SEQ ID NO: 1860) | (SEQ ID NO: |
| | 1859) | | 1861) |
| VL T30Y R53V | RASQDIYNYLN | YTSVLHS | QQGDTLPWT |
| (67BJN) | (SEQ ID NO: | (SEQ ID NO: 1863) | (SEQ ID NO: |
| | 1862) | | 1864) |
| | ABM numbering scheme | | |
| | CDR1 | CDR2 | CDR3 |
| VH W33D | GYTFTSYDMH | RIDPQSGGIK | WDYGGYFDV |
| (67BJN) | (SEQ ID NO: | (SEQ ID NO: 1866) | (SEQ ID NO: |
| | 1865) | | 1867) |
| VL T30Y R53V | RASQDIYNYLN | YTSVLHS | QQGDTLPWT |
| (67BJN) | (SEQ ID NO: | (SEQ ID NO: 1869) | (SEQ ID NO: |
| | 1868) | | 1870) |
| | IMGT numbering scheme | | |
| | CDR1 | CDR2 | CDR3 |
| VH W33D | GYTFTSYD | IDPQSGGI | ARWDYGGYFDV |
| (67BJN) | (SEQ ID NO: | (SEQ ID NO: 1872) | (SEQ ID NO: |
| | 1871) | | 1873) |
| VL T30Y R53V | QDIYNY | YTS | QQGDTLPWT |
| (67BJN) | (SEQ ID NO: | (SEQ ID NO: 1875) | (SEQ ID NO: |
| | 1874) | | 1876) |
| 68BJN | Kabat numbering scheme | | |
| V-region ID | CDR1 | CDR2 | CDR3 |
| VH H35Q S55D | SYWMQ | RIDPQDGDIKYAQKFQG | YDYGGYFDV |
| S57D W99Y | (SEQ ID NO: | (SEQ ID NO: 1878) | (SEQ ID NO: |
| (68BJN) | 1877) | | 1879) |
| VL T30Y R53V | RASQDIYNYLN | YTSVLHS | QQGDTLPWT |
| (68BJN) | (SEQ ID NO: | (SEQ ID NO: 1881) | (SEQ ID NO: |
| | 1880) | | 1882) |
| | Chothia numbering scheme | | |
| | CDR1 | CDR2 | CDR3 |
| VH H35Q S55D | GYTFTSY | DPQDGD | YDYGGYFDV |
| 357D W99Y | (SEQ ID NO: | (SEQ ID NO: 1884) | (SEQ ID NO: |
| (68BJN) | 1883) | | 1885) |
| VL T30Y R53V | RASQDIYNYLN | YTSVLHS | QQGDTLPWT |
| (68BJN) | (SEQ ID NO: 1886) | (SEQ ID NO: 1887) | (SEQ ID NO: 1888) |
| | ABM numbering scheme | | |
| | CDR1 | CDR2 | CDR3 |
| VH H35Q S55D | GYTFTSYWMQ | RIDPQDGDIK | YDYGGYFDV |
| S57D W99Y | (SEQ ID NO: | (SEQ ID NO: 1890) | (SEQ ID NO: |
| (68BJN) | 1889) | | 1891) |
| VL T30Y R53V | RASQDIYNYLN | YTSVLHS | QQGDTLPWT |
| (68BJN) | (SEQ ID NO: | (SEQ ID NO: 1893) | (SEQ ID NO: |
| | 1892) | | 1894) |
| | iMGT numbering scheme | | |
| | CDR1 | CDR2 | CDR3 |
| VH H35Q S55D | GYTFTSYW | IDPQDGDI | ARYDYGGYFDV |
| S57D W99Y | (SEQ ID NO: | (SEQ ID NO: 1896) | (SEQ ID NO: |
| (68BJN) | 1895) | | 1897) |
| VL T30Y R53V | QDIYNY | YTS | QQGDTLPWT |
| (68BJN) | (SEQ ID NO: | (SEQ ID NO: 1899) | (SEQ ID NO: |
| | 1898) | | 1900) |
| 69BJN | Kabat numbering scheme | | |
| V-region ID | CDR1 | CDR2 | CDR3 |
| VH VJ33D I58T | SYDMH | RIDPQSGGTKYAQKFQG | WDYGGYFDV |
| (69BJN) | (SEQ ID NO: | (SEQ ID NO: 1902) | (SEQ ID NO: |
| | 1901) | | 1903) |
| VL T30Y R53V | RASQDIYNYLN | YTSVLHS | QQGDTLPWT |
| (69BJN) | (SEQ ID NO: | (SEQ ID NO: 1905) | (SEQ ID NO: |
| | 1904) | | 1906) |
| | Chothia numbering scheme | | |
| | CDR1 | CDR2 | CDR3 |
| VH W33D I58T | GYTFTSY | DPQSGG | WDYGGYFDV |
| (69BJN) | (SEQ ID NO: | (SEQ ID NO: 1908) | (SEQ ID NO: |
| | 1907) | | 1909) |
| VL T30Y R53V | RASQDIYNYLN | YTSVLHS | QQGDTLPWT |
| (69BJN) | (SEQ ID NO: | (SEQ ID NO: 1911) | (SEQ ID NO: |
| | 1910) | | 1912) |
| | ABM numbering scheme | | |
| | CDR1 | CDR2 | CDR3 |
| VH W33D I58T | GYTFTSYDMH | RIDPQSGGTK | WDYGGYFDV |
| (69BJN) | (SEQ ID NO: | (SEQ ID NO: 1914) | (SEQ ID NO: |
| | 1913) | | 1915) |
| VL T30Y R53V | RASQDIYNYLN | YTSVLHS | QQGDTLPWT |
| (69BJN) | (SEQ ID NO: | (SEQ ID NO: 1917) | (SEQ ID NO: |
| | 1916) | | 1918) |
| | IMGT numbering scheme | | |
| | CDR1 | CDR2 | CDR3 |
| VH W33D I58T | GYTFTSYD | IDPQSGGT | ARWDYGGYFDV |
| (69BJN) | (SEQ ID NO: | (SEQ ID NO: 1920) | (SEQ ID NO: |
| | 1919) | | 1921) |
| VL T30Y R53V | QDIYNY | YTS | QQGDTLPWT |
| (69BJN) | (SEQ ID MO: | (SEQ ID NO: 1923) | (SEQ ID NO: |
| | 1922) | | 1924) |
| 71BJN | Kabat numbering scheme | | |
| V-region ID | CDR1 | CDR2 | CDR3 |
| VH W33D S55T | SYDMH | RIDPQTGDIKYAQKFQG | WDYGGQFDV |
| G57D Y104Q | (SEQ ID NO: | (SEQ ID NO: 1926) | (SEQ ID NO: |
| (71BJN) | 1925) | | 1927) |
| VL T30Y R53G | RASQDIYNYLN | YTSGLHS | QQGDTLPWT |
| (71BJN) | (SEQ ID NO: | (SEQ ID NO: 1929) | (SEQ ID NO: |
| | 1928) | | 1930) |
| | Chothia numbering scheme | | |
| | CDR1 | CDR2 | CDR3 |
| VH W33D S55T | GYTFTSY | DPQTGD | WDYGGQFDV |
| G57D Y104Q | (SEQ ID NO: | (SEQ ID NO: 1932) | (SEQ ID NO: |
| (71BJN) | 1931) | | 1933) |
| VL T30Y R53G | RASQDIYNYLN | YTSGLHS | QQGDTLPWT |
| (71BJN) | (SEQ ID NO: | (SEQ ID NO: 1935) | (SEQ ID NO: |
| | 1934) | | 1936) |
| | ABM numbering scheme | | |
| | CDR1 | CDR2 | CDR3 |
| VH W33D S55T | GYTFTSYDMH | RIDPQTGDIK | WDYGGQFDV |
| G57D Y104Q | (SEQ ID NO: | (SEQ ID NO: 1938) | (SEQ ID NO: |
| (71BJN) | 1937) | | 1939) |
| VL T30Y R53G | RASQDIYNYLN | YTSGLHS | QQGDTLPWT |
| (71BJN) | (SEQ ID NO: | (SEQ ID NO: 1941) | (SEQ ID NO: |
| | 1940) | | 1942) |
| | IMGT numbering scheme | | |
| | CDR1 | CDR2 | CDR3 |
| VH W33D S55T | GYTFTSYD | IDPQTGDI | ARWDYGGQFDV |
| G57D Y104Q | (SEQ ID NO: | (SEQ ID NO: 1944) | (SEQ ID NO: |
| (71BJN) | 1943) | | 1945) |
| VL T30Y R53G | QDIYNY | YTS | QQGDTLPWT |
| (71BJN) | (SEQ ID NO: | (SEQ ID NO: 1947) | (SEQ ID NO: |
| | 1946) | | 1948) |
| 72BJN | Kabat numbering scheme | | |
| V-region ID | CDR1 | CDR2 | CDR3 |
| VH S31Q W33D | QYDMH | RIDPQSGGIKYAQKFQG | YDYGGYFDV |
| W99Y (72BJN} | (SEQ ID NO: | (SEQ ID NO: 1950) | (SEQ ID NO: |
| | 1949) | | 1951) |
| VL T30Y R53I | RASQDIYNYLN | YTSILHS | QQGDTLPWT |
| (72BJN) | (SEQ ID NO: | (SEQ ID NO: 1953) | (SEQ ID NO: |
| | 1952) | | 1954) |
| | Chothia numbering scheme | | |
| | CDR1 | CDR2 | CDR3 |
| VH S31Q W33D | GYTFTQY | DPQSGG | YDYGGYFDV |
| W99Y (72BJN) | (SEQ ID NO: | (SEQ ID NO: 1956) | (SEQ ID NO: |
| | 1955) | | 1957) |
| VL T30Y R53I | RASQDIYNYLN | YTSILHS | QQGDTLPWT |
| (72BJN) | (SEQ ID NO: | (SEQ ID NO: 1959) | (SEQ ID NO: |
| | 1958) | | 1960) |
| | ABM numbering scheme | | |
| | CDR1 | CDR2 | CDR3 |
| VH S31Q W33D | GYTFTQYDMH | RIDPQSGGIK | YDYGGYFDV |
| W99Y (72BJN) | (SEQ ID NO: | (SEQ ID NO: 1962) | (SEQ ID NO: |
| | 1961) | | 1963) |
| VL T30Y R53I | RASQDIYNYLN | YTSILHS | QQGDTLPWT |
| (72BJN) | (SEQ ID NO: | (SEQ ID NO: 1965) | (SEQ ID NO: |
| | 1964) | | 1966) |
| | IMGT numbering scheme | | |
| | CDR1 | CDR2 | CDR3 |
| VH S31Q W33D | GYTFTQYD | IDPQSGGI | ARYDYGGYFDV |
| W99Y (72BJN) | (SEQ ID MO: | (SEQ ID NO: 1968) | (SEQ ID NO: |
| | 1967) | | 1969) |
| VL T30Y R53I | QDIYNY | YTS | QQGDTLPWT |
| (72BJN) | (SEQ ID NO: | (SEQ ID NO: 1971) | (SEQ ID NO: |
| | 1970) | | 1972) |
| 73BJN | Kabat numbering scheme | | |
| V-region ID | CDR1 | CDR2 | CDR3 |
| VH W33D | SYDMH | RIDPQSGGIKYAQKFQG | WDYGGYFDV |
| (73BJN) | (SEQ ID NO: | (SEQ ID NO: 1974) | (SEQ ID NO: |
| | 1973) | | 1975) |
| VL T30Y | RASQDIYNYLN | YTSRLHS | QQGDTLPWT |
| (73BJN) | (SEQ ID NO: | (SEQ ID NO: 1977) | (SEQ ID NO: |
| | 1976) | | 1978) |
| | Chothia numbering scheme | | |
| | CDR1 | CDR2 | CDR3 |
| VH W33D | GYTFTSY | DPQSGG | WDYGGYFDV |
| (73BJN) | (SEQ ID NO: | (SEQ ID NO: 1980) | (SEQ ID NO: |
| | 1979) | | 1981) |
| VL T30Y | RASQDIYNYLN | YTSRLHS | QQGDTLPWT |
| (73BJN) | (SEQ ID NO: | (SEQ ID NO: 1983) | (SEQ ID NO: |
| | 1982) | | 1984) |
| | ABM numbering scheme | | |
| | CDR1 | CDR2 | CDR3 |
| VH W33D | GYTFTSYDMH | RIDPQSGGIK | WDYGGYFDV |
| (73BJN) | (SEQ ID NO: | (SEQ ID NO: 1986) | (SEQ ID NO: |
| | 1985) | | 1987) |
| VL T30Y | RASQDIYNYLN | YTSRLHS | QQGDTLPWT |
| (73BJN) | (SEQ ID NO: | (SEQ ID NO: 1989) | (SEQ ID NO: |
| | 1988) | | 1990) |
| | IMGT numbering scheme | | |
| | CDR1 | CDR2 | CDR3 |
| VH W33D | GYTFTSYD | IDPQSGGI | ARWDYGGYFDV |
| (73BJN) | (SEQ ID NO: | (SEQ ID NO: 1992) | (SEQ ID NO: |
| | 1991) | | 1993) |
| VL T30Y | QDIYNY | YTS | QQGDTLPWT |
| (73BJN) | (SEQ ID NO: | (SEQ ID NO: 1995) | (SEQ ID NO: |
| | 1994) | | 1996) |
| 74BJN | Kabat numbering scheme | | |
| V-region ID | CDR1 | CDR2 | CDR3 |
| VH S31Y W33D | YYDLH | RIDPQSGGIKYAQKFQG | WDYGGYFDV |
| M34L (74BJN) | (SEQ ID NO: | (SEQ ID NO: 1998) | (SEQ ID NO: |
| | 1997) | | 1999) |
| VL T30Y T93D | RASQDIYNYLNW | YTSRLHS | QQGDDIPWTF |
| L94I (74BJN) | (SEQ ID NO: | (SEQ ID NO: 2001) | (SEQ ID NO: |
| | 2000) | | 2002) |
| | Chothia numbering scheme | | |
| | CDR1 | CDR2 | CDR3 |
| VH S31Y W33D | GYTFTYY | DPQSG | WDYGGYFDV |
| M34L (74BJN) | (SEQ ID NO: | (SEQ ID NO: 2004) | (SEQ ID MO: |
| | 2003) | | 2005) |
| VL T30Y T93D | RASQDIYNYLNW | YTSRLHS | QQGDDIPWTF |
| L94I (74BJN) | (SEQ ID NO: | (SEQ ID NO: 2007) | (SEQ ID NO: |
| | 2006) | | 2008) |
| | ABM numbering scheme | | |
| | CDR1 | CDR2 | CDR3 |
| VH S31Y W33D | gytftyydlh | RIDPQSGGIK | WDYGGYFDV |
| M34L (74BJN) | (SEQ ID NO: | (SEQ ID NO: 2010) | (SEQ ID NO: |
| | 2009) | | 2011) |
| VL T30Y T93D | RASQDIYNYLNW | YTSRLHS | QQGDDIPWTF |
| L94I (74BJN) | (SEQ ID NO: | (SEQ ID NO: 2013) | (SEQ ID NO: |
| | 2012) | | 2014) |
| | IMGT numbering scheme | | |
| | CDR1 | CDR2 | CDR3 |
| VH S31Y W33D | GYTFTYYD | IDPQSGGI | ARWDYGGYFDV |
| M34L (74BJN) | (SEQ ID NO: | (SEQ ID NO: 2016) | (SEQ ID NO: |
| | 2015) | | 2017) |
| VL T30Y T93D | QDIYNYL | YTS | QQGDDIPWTF |
| L94I (74BJN) | (SEQ ID NO: | (SEQ ID NO: 2019) | (SEQ ID NO: |
| | 2018) | | 2020) |
| 75BJN | Kabat numbering scheme | | |
| V-region ID | CDR1 | CDR2 | CDR3 |
| VH S31K W33D | KYDMH | RIDPQDGGFKYAQKFQG | WDYGGYFDV |
| S55D I58F | (SEQ ID NO: | (SEQ ID NO: 2022) | (SEQ ID NO: |
| (75BJN) | 2021) | | 2023) |
| VL T30Y R53Y | RASQDIFNYLN | YTSYLHS | QQGDELPWT |
| T93E (75BJN) | (SEQ ID NO: | (SEQ ID NO: 2025) | (SEQ ID NO: |
| | 2024) | | 2026) |
| | Chothia numbering scheme | | |
| | CDR1 | CDR2 | CDR3 |
| VH S31K W33D | GYTFTKY | DPQDGG | WDYGGYFDV |
| S55D I58F | (SEQ ID NO: | (SEQ ID NO: 2028) | (SEQ ID NO: |
| (75BJN) | 2027) | | 2029) |
| VL T30Y R53Y | RASQDIFNYLN | YTSYLHS | QQGDELPWT |
| T93E (75BJN) | (SEQ ID NO: | (SEQ ID NO: 2031) | (SEQ ID NO: |
| | 2030) | | 2032) |
| | ABM numbering scheme | | |
| | CDR1 | CDR2 | CDR3 |
| VH S31K W33D | GYTFTKYDMH | RIDPQDGGFK | WDYGGYFDV |
| S55D I58F | (SEQ ID NO: | (SEQ ID NO: 2034) | (SEQ ID NO: |
| (75BJN) | 2033) | | 2035) |
| VL T30Y R53Y | RASQDIFNYLN | YTSYLHS | QQGDELPWT |
| T93E (75BJN) | (SEQ ID NO: | (SEQ ID NO: 2037) | (SEQ ID NO: |
| | 2036) | | 2038) |
| | IMGT numbering scheme | | |
| | CDR1 | CDR2 | CDR3 |
| VH S31K W33D | GYTFTKYD | IDPQDGGF | ARWDYGGYFDV |
| S55D I58F | (SEQ ID NO: | (SEQ ID NO: 2040) | (SEQ ID MO: |
| (75BJN) | 2039) | | 2041) |
| VL T30Y R53Y | QDIFNY | YTS | QQGDELPWT |
| T93E (75BJN) | (SEQ ID NO: | (SEQ ID NO: 2043) | (SEQ ID NO: |
| | 2042) | | 2044) |
| 76BJN | Kabat numbering scheme | | |
| V-region ID | CDR1 | CDR2 | CDR3 |
| VH S31Y W33D | YYDMH | RIDPYDGDIKYAQKFQG | WDYGGYFDV |
| Q54Y S55D | (SEQ ID NO: | (SEQ ID NO: 2046) | (SEQ ID NO: |
| G57D (76BJN) | 2045) | | 2047) |
| VL T30Y R53L | RASQDIYNYLN | YTSLLHS | QQGDELPWT |
| T93E (76BJN) | (SEQ ID NO: | (SEQ ID NO: 2049) | (SEQ ID NO: |
| | 2048) | | 2050) |
| | Chothia numbering scheme | | |
| | CDR1 | CDR2 | CDR3 |
| VH S31Y W33D | GYTFTYY | DPYDGD | WDYGGYFDV |
| Q54Y S55D | (SEQ ID NO: | (SEQ ID NO: 2052) | (SEQ ID NO: |
| G57D (76BJN) | 2051) | | 2053) |
| VL T30Y R53L | RASQDIYNYLN | YTSLLHS | QQGDELPWT |
| T93E (76BJN) | (SEQ ID NO: | (SEQ ID NO: 2055) | (SEQ ID NO: |
| | 2054) | | 2056) |
| | ABM numbering scheme | | |
| | CDR1 | CDR2 | CDR3 |
| VH S31Y W33D | GYTFTYYDMH | RIDPYDGDIK | WDYGGYFDV |
| Q54Y S55D | (SEQ ID NO: | (SEQ ID NO: 2058) | (SEQ ID NO: |
| G57D (76BJN) | 2057) | | 2059) |
| VL T30Y R53L | RASQDIYNYLN | YTSLLHS | QQGDELPWT |
| T93E (76BJN) | (SEQ ID NO: | (SEQ ID NO: 2061) | (SEQ ID NO: |
| | 2060) | | 2062) |
| | IMGT numbering scheme | | |
| | CDR1 | CDR2 | CDR3 |
| VH S31Y W33D | GYTFTYYD | IDPYDGDI | ARWDYGGYFDV |
| Q54Y S55D | (SEQ ID NO: | (SEQ ID NO: 2064) | (SEQ ID NO: |
| G57D (76BJN) | 2063) | | 2065) |
| VL T30Y R53L | QDIYNY | YTS | QQGDELPWT |
| T93E (76BJN) | (SEQ ID NO: | (SEQ ID NO: 2067) | (SEQ ID NO: |
| | 2066) | | 2068) |
| 77BJN | Kabat numbering scheme | | |
| V-region ID | CDR1 | CDR2 | CDR3 |
| VH W33D I58V | SYDMH | RIDPQSGGVKYAQKFQG | WDYGGYFDV |
| (77BJN) | (SEQ ID NO: | (SEQ ID NO: 2070) | (SEQ ID MO: |
| | 2069) | | 2071) |
| VL T30Y R53V | RASQDIYNYLN | YTSVLHS | QQGDELPWTF |
| T93E (77BJN) | (SEQ ID NO: | (SEQ ID NO: 2073) | (SEQ ID NO: |
| | 2072) | | 2074) |
| | Chothia numbering scheme | | |
| | CDR1 | CDR2 | CDR3 |
| VH W33D I58V | GYTFTSY | DPQSGG | WDYGGYFDV |
| (77BJN) | (SEQ ID NO: | (SEQ ID NO: 2076) | (SEQ ID NO: |
| | 2075) | | 2077) |
| VL T30Y R53V | RASQDIYNYLN | YTSVLHS | QQGDELPWTF |
| T93E (77BJN) | (SEQ ID NO: | (SEQ ID NO: 2079) | (SEQ ID NO: |
| | 2078) | | 2080) |
| | ABM numbering scheme | | |
| | CDR1 | CDR2 | CDR3 |
| VH W33D I58V | GYTFTSYDMH | RIDPQSGGVK | WDYGGYFDV |
| (77BJN) | (SEQ ID NO: | (SEQ ID NO: 2082) | (SEQ ID NO: |
| | 2081) | | 2083) |
| VL T30Y R53V | RASQDIYNYLN | YTSVLHS | QQGDELPWTF |
| T93E (77BJN) | (SEQ ID NO: 2084) | (SEQ ID NO: 2085) | (SEQ ID NO: 2086) |
| | IMGT numbering scheme | | |
| | CDR1 | CDR2 | CDR3 |
| VH W33D I58V | GytftsYD | IDPQSGGV | ARWDYGGYFDV |
| (77BJN) | (SEQ ID NO: | (SEQ ID NO: 2088) | (SEQ ID NO: |
| | 2087) | | 2089) |
| VL T30Y R53V | QDIYNY | YTS | QQGDELPWTF |
| T93E (77BJN) | (SEQ ID NO: | (SEQ ID NO: 2091) | (SEQ ID NO: |
| | 2090) | | 2092) |
| 78BJN | Kabat numbering scheme | | |
| V-region ID | CDR1 | CDR2 | CDR3 |
| VH W33D K59R | SYDMH | RIDPQSGGIRYAQKFQG | WDYGGYFDV |
| (78BJN) | (SEQ ID NO: | (SEQ ID NO: 2094) | (SEQ ID NO: |
| | 2093) | | 2095) |
| VL T30Y R53N | RASQDIYNYLN | YTSNLHS | QQGDELPWT |
| T93E (78BJN) | (SEQ ID NO: | (SEQ ID NO: 2097) | (SEQ ID NO: |
| | 2096) | | 2098) |
| | Chothia numbering scheme | | |
| | CDR1 | CDR2 | CDR3 |
| VH W33D I<59R | GYTFTSY | DPQSGG | WDYGGYFDV |
| (78BJN) | (SEQ ID NO: | (SEQ ID NO: 2100) | (SEQ ID NO: |
| | 2099) | | 2101) |
| VL T30Y R53N | RASQDIYNYLN | YTSNLHS | QQGDELPWT |
| T93E (78BJN) | (SEQ ID NO: | (SEQ ID NO: 2103) | (SEQ ID NO: |
| | 2102) | | 2104) |
| | ABM numbering scheme | | |
| | CDR1 | CDR2 | CDR3 |
| VH W33D K59R | GYTFTSYDMH | RIDPQSGGIR | WDYGGYFDV |
| (78BJN) | (SEQ ID NO: | (SEQ ID NO: 2106) | (SEQ ID MO: |
| | 2105) | | 2107) |
| VL T30Y R53N | RASQDIYNYLN | YTSNLHS | QQGDELPWT |
| T93E (78BJN) | (SEQ ID NO: | (SEQ ID NO: 2109) | (SEQ ID NO: |
| | 2108) | | 2110) |
| | IMGT numbering scheme | | |
| | CDR1 | CDR2 | CDR3 |
| VH W33D K59R | GYTFTSYD | IDPQSGGI | ARWDYGGYFDV |
| (78BJN) | (SEQ ID NO: | (SEQ ID NO: 2112) | (SEQ ID NO: |
| | 2111) | | 2113) |
| VL T30Y R53N | QDIYNY | YTS | QQGDELPWT |
| T93E (78BJN) | (SEQ ID NO: | (SEQ ID NO: 2115) | (SEQ ID NO: |
| | 2114) | | 2116) |
| 79BJN | Kabat numbering scheme | | |
| V-region ID | CDR1 | CDR2 | CDR3 |
| VH W33D S55D | SYDMH | RIDPQDGDVKYAQKFQG | WDYGGYFDV |
| S57D I58V | (SEQ ID NO: | (SEQ ID NO: 2118) | (SEQ ID NO: |
| (79BJN) | 2117) | | 2119) |
| VL T30Y R53G | RASQDIYNYLN | YTSGLHS | QQGDELPWT |
| T93E (79BJN) | (SEQ ID NO: | (SEQ ID NO: 2121) | (SEQ ID NO: |
| | 2120) | | 2122) |
| | Chothia numbering scheme | | |
| | CDR1 | CDR2 | CDR3 |
| VH W33D S55D | GYTFTSY | DPQDGD | WDYGGYFDV |
| S57D I58V | (SEQ ID NO: | (SEQ ID NO: 2124) | (SEQ ID NO: |
| (79BJN) | 2123) | | 2125) |
| VL T30Y R53G | RASQDIYNYLN | YTSGLHS | QQGDELPWT |
| T93E (79BJN) | (SEQ ID NO: | (SEQ ID NO: 2127) | (SEQ ID NO: |
| | 2126) | | 2128) |
| | ABM numbering scheme | | |
| | CDR1 | CDR2 | CDR3 |
| VH W33D S55D | GYTFTSYDMH | RIDPQDGDVK | WDYGGYFDV |
| S57D I58V | (SEQ ID NO: | (SEQ ID NO: 2130) | (SEQ ID NO: |
| (79BJN) | 2129) | | 2131) |
| VL T30Y R53G | RASQDIYNYLN | YTSGLHS | QQGDELPWT |
| T93E (79BJN) | (SEQ ID NO: | (SEQ ID NO: 2133) | (SEQ ID NO: |
| | 2132) | | 2134) |
| | IMGT numbering scheme | | |
| | CDR1 | CDR2 | CDR3 |
| VH W33D S55D | GYTFTSYD | IDPQDGDV | ARWDYGGYFDV |
| S57D I58V | (SEQ ID NO: | (SEQ ID NO: 2136) | (SEQ ID NO: |
| (79BJN) | 2135) | | 2137) |
| VL T30Y R53G | QDIYNY | YTS | QQGDELPWT |
| T93E (79BJN) | (SEQ ID NO: | (SEQ ID NO: 2139) | (SEQ ID NO: |
| | 2138) | | 2140) |
| 80BJN | Kabat numbering scheme | | |
| V-region ID | CDR1 | CDR2 | CDR3 |
| VH W33D K59R | SYDMH | RIDPQSGGIRYAQKFQG | WDYGGQFDV |
| Y104Q (80BJN) | (SEQ ID NO: | (SEQ ID NO: 2142) | (SEQ ID NO: |
| | 2141) | | 2143) |
| VL T30Y R53I | RASQDIYNYLN | YTSILHS | QQGDTLPWT |
| (80BJN) | (SEQ ID NO: | (SEQ ID NO: 2145) | (SEQ ID NO: |
| | 2144) | | 2146) |
| | Chothia numbering scheme | | |
| | CDR1 | CDR2 | CDR3 |
| VH W33D K59R | GYTFTSY | DPQSGG | WDYGGQFDV |
| Y104Q (80BJN) | (SEQ ID NO: | (SEQ ID NO: 2148) | (SEQ ID NO: |
| | 2147) | | 2149) |
| VL T30Y R53I | RASQDIYNYLN | YTSILHS | QQGDTLPWT |
| (80BJN) | (SEQ ID NO: 2150) | (SEQ ID NO: 2151) | (SEQ ID NO: 2152) |
| | ABM numbering scheme | | |
| | CDR1 | CDR2 | CDR3 |
| VH W33D K59R | GYTFTSYDMH | RIDPQSGGIR | WDYGGQFDV |
| Y104Q (80BJN) | (SEQ ID NO: | (SEQ ID NO: 2154) | (SEQ ID NO: |
| | 2153) | | 2155) |
| VL T30Y R53I | RASQDIYNYLN | YTSILHS | QQGDTLPWT |
| (80BJN) | (SEQ ID NO: | (SEQ ID NO: 2157) | (SEQ ID NO: |
| | 2156) | | 2158) |
| | iMGT numbering scheme | | |
| | CDR1 | CDR2 | CDR3 |
| VH W33D K59R | GYTFTSYD | IDPQSGGI | ARWDYGGQFDV |
| Y104Q (80BJN) | (SEQ ID NO: | (SEQ ID NO: 2160) | (SEQ ID NO: |
| | 2159) | | 2161) |
| VL T30Y R53I | QDIYNY | YTS | QQGDTLPWT |
| (80BJN) | (SEQ ID NO: | (SEQ ID NO: 2163) | (SEQ ID NO: |
| | 2162) | | 2164) |
| 81BJN | Kabat numbering scheme | | |
| V-region ID | CDR1 | CDR2 | CDR3 |
| VH S31E W33D | EYDMH | RIDPQSGGIRYAQKFQG | WDYGGQFDA |
| K59R Y104Q | (SEQ ID NO: | (SEQ ID NO: 2166) | (SEQ ID NO: |
| V107A (81BJN) | 2165) | | 2167) |
| VL T30Y R53V | RASQDIYNYLN | YTSVLHS | QQGDTLPWT |
| (81BJN) | (SEQ ID NO: | (SEQ ID NO: 2169) | (SEQ ID NO: |
| | 2168) | | 2170) |
| | Chothia numbering scheme | | |
| | CDR1 | CDR2 | CDR3 |
| VH S3IE W33D | GYTFTEY | DPQSGG | WDYGGQFDA |
| K59R Y104Q | (SEQ ID NO: | (SEQ ID NO: 2172) | (SEQ ID NO: |
| V107A (81BJN) | 2171) | | 2173) |
| VL T30Y R53V | RASQDIYNYLN | YTSVLHS | QQGDTLPWT |
| (81BJN) | (SEQ ID NO: | (SEQ ID NO: 2175) | (SEQ ID NO: |
| | 2174) | | 217 6) |
| | ABM numbering scheme | | |
| | CDR1 | CDR2 | CDR3 |
| VH S31E W33D | GYTFTEYDMH | RIDPQSGGIR | WDYGGQFDA |
| K59R Y104Q | (SEQ ID NO: | (SEQ ID NO: 2178) | (SEQ ID NO: |
| V107A (81BJN) | 2177) | | 2179) |
| VL T30Y R53V | RASQDIYNYLN | YTSVLHS | QQGDTLPWT |
| (81BJN) | (SEQ ID NO: | (SEQ ID NO: 2181) | (SEQ ID NO: |
| | 2180) | | 2182) |
| | IMGT numbering scheme | | |
| | CDR1 | CDR2 | CDR3 |
| VH S31E W33D | GYTFTEYD | IDPQSGGI | ARWDY GGQFDA |
| K59R Y104Q | (SEQ ID NO: | (SEQ ID NO: 2184) | (SEQ ID NO: |
| V107A (81BJN) | 2183) | | 2185) |
| VL T30Y R53V | QDIYNY | YTS | QQGDTLPWT |
| (81BJN) | (SEQ ID NO: | (SEQ ID NO: 2187) | (SEQ ID NO: |
| | 2186) | | 2188) |
| 82BJN | Kabat numbering scheme | | |
| V-region ID | CDR1 | CDR2 | CDR3 |
| VH W33Y H35N | SYYMN | RIDPQSGGVKYAQKFQG | WHYGGQFDV |
| I58V D100H | (SEQ ID NO: | (SEQ ID NO: 2190) | (SEQ ID NO: |
| Y104Q (82BJN) | 2189) | | 2191) |
| VL T30Y R53G | RASQDIYNYLN | YTSGLHS | QQGDTLPWT |
| (82BJN) | (SEQ ID NO: | (SEQ ID NO: 2193) | (SEQ ID NO: |
| | 2192) | | 2194) |
| | Chothia numbering scheme | | |
| | CDR1 | CDR2 | CDR3 |
| VH VJ33Y H35N | GYTFTSY | IDPQSGGV | WHYGGQFDV |
| I58V D100H | (SEQ ID NO: | (SEQ ID NO: 2196) | (SEQ ID NO: |
| Y104Q (82BJN) | 2195) | | 2197) |
| VL T30Y R53G | RASQDIYNYLN | YTSGLHS | QQGDTLPWT |
| (82BJN) | (SEQ ID NO: | (SEQ ID NO: 2199) | (SEQ ID NO: |
| | 2198) | | 2200) |
| | ABM numbering scheme | | |
| | CDR1 | CDR2 | CDR3 |
| VH W33Y H35N | GYTFTSYYMN | RIDPQSGGVK | WHYGGQFDV |
| I58V D100H | (SEQ ID NO: | (SEQ ID NO: 2202) | (SEQ ID NO: |
| Y104Q (82BJN) | 2201) | | 2203) |
| VL T30Y R53G | RASQDIYNYLN | YTSGLHS | QQGDTLPWT |
| (82BJN) | (SEQ ID NO: | (SEQ ID NO: 2205) | (SEQ ID NO: |
| | 2204) | | 2206) |
| | IMGT numbering scheme | | |
| | CDR1 | CDR2 | CDR3 |
| VH W33Y H35N | GYTFTSYY | IDPQSGGV | ARWHYGGQFDV |
| I58V D100H | (SEQ ID NO: | (SEQ ID NO: 2208) | (SEQ ID NO: |
| Y104Q (82BJN) | 2207) | | 2209) |
| VL T30Y R53G | QDIYNY | YTS | QQGDTLPWT |
| (82BJN) | (SEQ ID NO: | (SEQ ID NO: 2211) | (SEQ ID NO: |
| | 2210) | | 2212) |

### Example 13: Next Generation Sequencing (NS) Analysis of 20E6 Variants from Yeast Selection Outputs

To more comprehensively understand the CDR residue changes that are selected from the different sorting gates for better binding to LAP-TGFb1 protein by yeast display, investigators isolated 20E6 variant sequences from each of the outputs and subjected them to next generation sequences. It was thought that the sequences derived from the NGS analysis may provide additional information on critical residues at each CDR position that contribute to improved LAP-TGFb1 binding individually or in combination. The increased available sequences allowed the selection of 20E6 variants that had the most optimal binding to LAP-TGFb1 while minimizing impact on potentially increased immunogenicity or developability.

### Example 14: Yeast Display Mutants Binding Human LAP-TGFb Isoforms 1, 2, and 3

This Example describes the isoform specificity of the yeast display mutants to bind to human LAP-TGFβ isoforms 1, 2, and 3 using surface plasmon resonance. A Series S CM4 sensor chip (GE Healthcare, catalog BR100534) was immobilized with an anti-human Fc capture antibody following the kit protocol (GE Healthcare, catalog BR100839) on a Biacore T200 instrument with 1× HBS-EP+ (Teknova, catalog H8022). Kinetic binding interactions between human LAP-TGFβ isoform 1 and yeast display mutants were analyzed in 1× HBS-EP+ with 0.1 mg/mL BSA (Jackson Immunoresearch, catalog 001-000-162) at 25° C. Approximately 20-40 RU amounts of human LAP-TGFβ-Fc isoform 1 were captured to the anti-human Fc surface followed by injection of 1:4 serially diluted Fab from 1000 nM to 3.91 nM (20E6), 100 nM to 0.39 nM (mutants 55BJN-66BJN), 25 nM to 0.097 nM (mutants 67BJN-69BJN, 71BJN-79BJN), and 1:3 serially diluted Fab from 6 nM to 0.07 nM (mutants 80BJN-82BJN) and including a negative control 0 nM Fab. Binding specificity experiments between human LAP-TGFβ isoforms 2 and 3 and yeast display mutants were performed in 1× HBS-EP+ with 0.1 mg/mL BSA at 25° C. Approximately 20-40 RU amounts of human LAP-TGFβ2 and 3 were captured to the anti-human Fc surface followed by injection of 1000 nM Fab including a negative control 0 nM Fab. The binding data were double referenced by subtraction of signal from a reference (capture surface only) flow cell and the negative control 0 nM Fab injection. Binding rate constants were determined by fitting the data with a 1:1 binding model (GE Healthcare Biacore T200 Evaluation software 3.0).

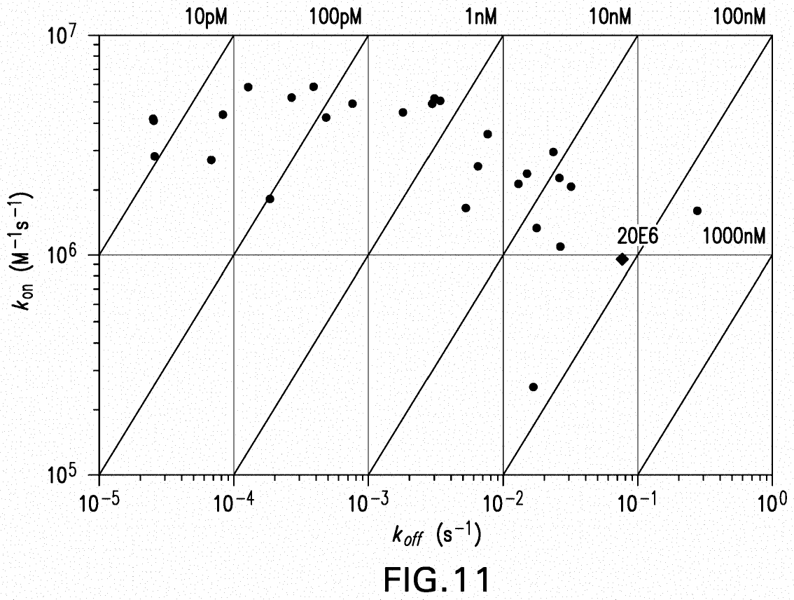

As shown in FIG. 11 and Table 44, the 20E6 antibody bound to human LAP-TGFβ1 with nanomolar affinity, but no appreciable signal increase was observed for human LAP-TGFβ isoformn 2 or 3. The 20E6 variants demonstrated affinities which span over four logs of improvement in binding affinity to human LAP-TGFβ1 over the non-affinity matured 20E6 antibody. These affinity improvements largely resulted from improved, or slowed, off-rates however several mutants did exhibit improved on-rates, up to 6-fold, over 20E6.

TABLE 44
Binding parameters for 20E6 and yeast display mutants binding to human LAP-TGFβ isoforms 1, 2, and 3

| | | LAP-TGFβ1 | | | | | |
|---|---|---|---|---|---|---|---|
| | | | | | Fold | LAP-TGFβ2 | LAP-TGFβ3 |
| | Sorting | kₒₙ | k_{off} | K_{D} | improvement | K_{D} | K_{D} |
| ID | population | (M⁻¹s⁻¹) | (s⁻¹) | (M) | in K_{D} | (M) | (M) |
| 20E6 (80BGJ) | Parent | 0.96 × 10⁶ | 7.69 × 10⁻² | 7.99 × 10⁻⁸ | 1 | NB | NB |
| 55BJN | 2 hr P3 | 1.10 × 10⁵ | 2.67 × 10⁻² | 2.44 × 10⁻⁸ | 3 | NB | NB |
| 56BJN | 2 hr P3 | 2.53 × 10⁶ | 1.67 × 10⁻² | 6.63 × 10⁻⁸ | 1 | NB | NB |
| 57BJN | 2 hr P4 | 1.33 × 10⁶ | 1.78 × 10⁻² | 1.34 × 10⁻⁸ | 6 | NB | NB |
| 58BJN | 2 hr P3 | 2.35 × 10⁶ | 1.51 × 10⁻² | 6.42 × 10⁻⁹ | 13 | NB | NB |
| 59BJN | 2 hr P4 | 2.24 × 10⁶ | 2.63 × 10⁻² | 1.17 × 10⁻⁸ | 7 | NB | NB |
| 60BJN | 2 hr P3 | 2.54 × 10⁶ | 6.53 × 10⁻³ | 2.57 × 10⁻⁹ | 31 | NB | NB |
| 61BJN | 2 hr P4 | 1.59 × 10⁶ | 2.79 × 10⁻¹ | 1.75 × 10⁻⁷ | 1 | NB | NB |
| 62BJN | 2 hr P4 | 2.05 × 10⁶ | 3.20 × 10⁻² | 1.56 × 10⁻⁸ | 5 | NB | NB |
| 63BJN | 2 hr P3 | 1.64 × 10⁶ | 5.30 × 10⁻³ | 3.23 × 10⁻⁹ | 25 | NB | NB |
| 64BJN | 2 hr P3 | 3.57 × 10⁶ | 7.70 × 10⁻³ | 2.15 × 10⁻⁹ | 37 | NB | NB |
| 65BJN | 2 hr P4 | 2.94 × 10⁶ | 2.38 × 10⁻² | 8.12 × 10⁻⁹ | 10 | NB | NB |
| 66BJN | 2 hr P3 | 2.11 × 10⁶ | 1.31 × 10⁻² | 6.21 × 10⁻⁹ | 13 | NB | NB |
| 67BJN | 2 hr P2 | 4.87 × 10⁶ | 7.62 × 10⁻⁴ | 1.56 × 10⁻¹⁰ | 512 | NB | NB |
| 68BJN | 2 hr P1 | 4.36 × 10⁶ | 8.34 × 10⁻⁵ | 1.91 × 10⁻¹¹ | 4205 | NB | NB |
| 69BJN | 2 hr P1 | 4.25 × 10⁶ | 4.88 × 10⁻⁴ | 1.15 × 10⁻¹⁰ | 695 | NB | NB |
| 71BJN | 2 hr P1 | 2.71 × 10⁶ | 6.80 × 10⁻⁵ | 2.51 × 10⁻¹¹ | 3196 | NB | NB |
| 72BJN | 2 hr P2 | 4.85 × 10⁶ | 2.98 × 10⁻³ | 6.15 × 10⁻¹⁰ | 130 | NB | NB |
| 73BJN | 2 hr P2 | 5.15 × 10⁶ | 3.10 × 10⁻³ | 6.01 × 10⁻¹⁰ | 133 | NB | NB |
| 74BJN | 2 hr P2 | 4.45 × 10⁶ | 1.81 × 10⁻³ | 4.08 × 10⁻¹⁰ | 196 | NB | NB |
| 75BJN | 2 hr P1 | 5.05 × 10⁶ | 3.43 × 10⁻³ | 6.78 × 10⁻¹¹ | 1178 | NB | NB |
| 76BJN | 2 hr P1 | 1.80 × 10⁶ | 1.86 × 10⁻⁴ | 1.04 × 10⁻¹⁰ | 768 | NB | NB |
| 77BJN | 2 hr P1 | 5.22 × 10⁶ | 2.70 × 10⁻⁴ | 5.17 × 10⁻¹¹ | 1545 | NB | NB |
| 78BJN | 2 hr P1 | 5.52 × 10⁶ | 3.93 × 10⁻⁴ | 7.13 × 10⁻¹¹ | 1121 | NB | NB |
| 79BJN | 2 hr P1 | 5.84 × 10⁶ | 1.28 × 10⁻⁴ | 2.20 × 10⁻¹¹ | 3632 | NB | NB |
| 80BJN | 12 hr P1 | 4.15 × 10⁶ | 2.52 × 10⁻⁵ | 6.07 × 10⁻¹² | 13163 | NB | NB |
| 81BJN | 12 hr P1 | 4.08 × 10⁶ | 2.56 × 10⁻⁵ | 6.29 × 10⁻¹² | 12702 | NB | NB |
| 82BJN | 12 hr P1 | 2.81 × 10⁶ | 2.60 × 10⁻⁵ | 9.28 × 10⁻¹² | 8610 | NB | NB |

NB: No binding observed

### Example 15: Kinetic exclusion assay affinity assessment of a yeast display mutant 69BJN binding human LAP-TGFβ1

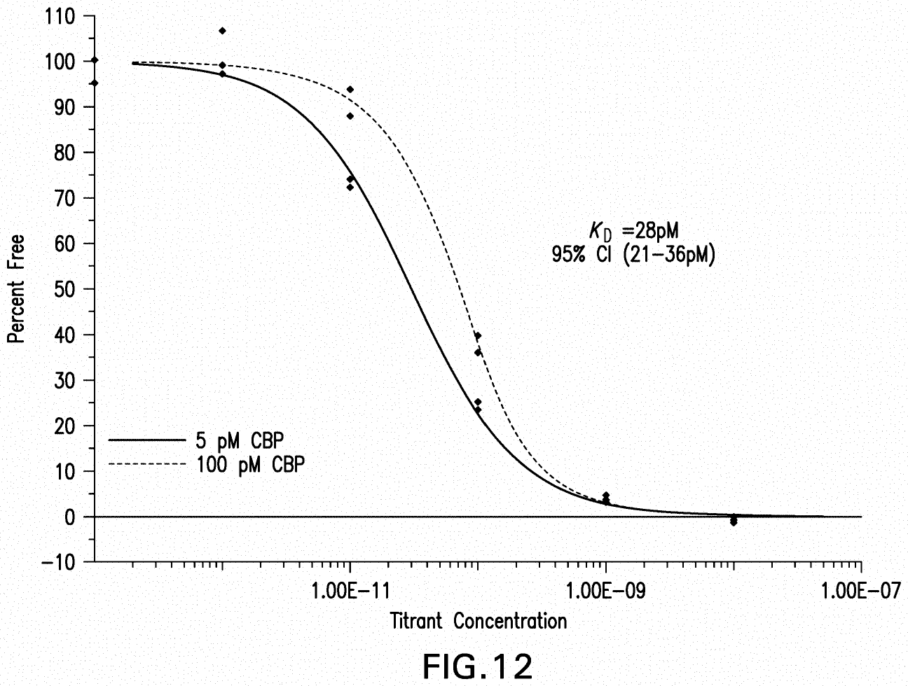

An orthogonal kinetic exclusion assay (KinExA) method was used in this study to measure the affinity of an affinity-matured anti-LAP-TGF β1 Fab 69BJN (referred to as the constant binding partner, CBP) and human LAP-TGFβ1 (referred to as the titrant). To determine the free CBP concentration in solution, PMMA beads (Sapidyne, catalog 440176) were coated with human LAP-TGFβ1 and then blocked with BSA (Jackson Immunoresearch, catalog 001-000-162). Two equilibrium experiments were prepared in assay buffer (1× PBS, 1 mg/mL BSA, and 0.05% NaN₃) with 5 pM or 100 pM of 69BJN as CBP followed by mixing and equilibrating with 1:10 serially diluted human LAP-TGFβ1 from 10 nM to 1 pM including a negative control 0 nM. The KinExA method was performed as follows: PMMA beads were loaded into the flow cell, a single concentration of titrant human LAP-TGFβ1 at equilibrium with 69BJN CBP was flowed over the flow cell, free CBP bound by PMMA beads was detected with rabbit anti-human IgG, F(ab′)₂ fragment specific-647 conjugate (Jackson Immunoresearch, catalog 309-005-006). This method was repeated in duplicate over the entire concentration series for each of the two different equilibrium series. The binding signals were analyzed with KinExA Pro software (version 4.4.26) where the free CBP binding signals were converted into percent free response and plotted against the titrant concentration series. The equilibrium K_{D} was determined by using the titrant concentration as a reference and calculating percent activity of the ligand (CBP). As shown in FIG. 12, the equilibrium affinity analysis by KinExA shows that anti-human LAP-TGFβ1 Fab 69BJN bound to human LAP-TGFβ1 with a K_{D} of 28 pM (95% confidence interval of 21-36 pM).

### Example 16:: Selection of 15 Affinity-Matured 20E6 Variants for IgG Protein Expression

This Examples was performed to better assess the effects of 20E6 affinity improvement on in vitro and in vivo assays across a wide affinity range while increasing sequence identity to human antibodies by incorporating LCDR2 E55/T56 human germline mutations as well as optimizing pI value of the IgG protein to below 9.0. Investigators selected 15 additional 20E6 variants for IgG expression. In further examples, investigators analyzed if the IgG proteins bound to human LAP-TGFb1 at both pH 7.4 and pH 6.0. The selections were also based on choosing sequences that have as few CDR residue changes as possible. The sequences are listed in Tables 45A-C. An expanded table listing the CDRs for these 15 antibodies by different numbering schemes is also provided in Table 45A.

The further studies were performed with the affinity matured 20E6 antibody and antigen-binding fragments thereof such that the molecules were engineered to include further modifications to CDR residues within the variable domains of the humanized affinity matured monoclonal antibodies and antigen-binding fragments thereof described above. Fifteen modified affinity matured antibodies were named 06BLM, 07BLM, 08BLM, 09BLM, 10BLM, 11BLM, 12BLM, 13BLM, 14BLM, 15BLM, 16BLM, 17BLM, 18BLM, 19BLM, and 20BLM

The modifications were analyzed for ability to increase the affinity of the antibody or antigen binding fragment thereof. Table 45A, Table 45B, and Table 45C list the CDR sequences (i.e., Kabat, Chothia, ABM, and IMGT numbering schemes), VH and VL amino acid sequences, and heavy chain and light chain sequences, respectively, of the modified affinity matured antibodies 06BLM, 07BLM, 08BLM, 09BLM, 10BLM, 11BLM, 12BLM, 13BLM, 14BLM, 15BLM, 16BLM, 17BLM, 18BLM, 19BLM, and 20BLM.

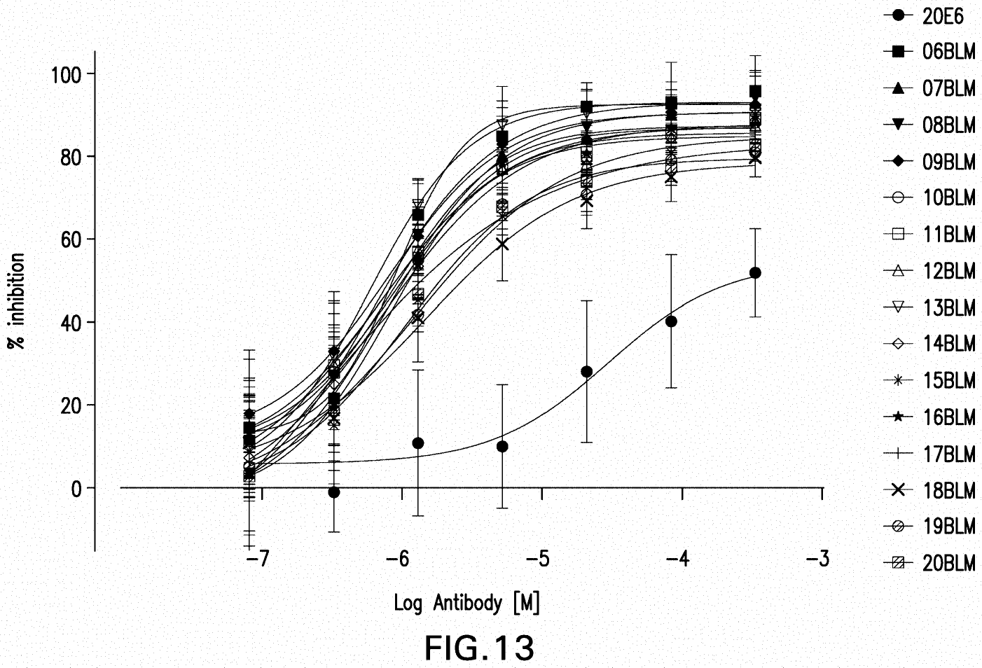

Functional EC50 inhibition data for the parental 20E6 humanized antibody and the 15 selected affinity matured humanized antibodies are shown in Table 46 and FIG. 13.

TABLE 45A
CDR amino acid sequences for the 15 selected affinity maturation humanized
antibodies

| | Kabat numbering scheme | | |
|---|---|---|---|
| V-region ID | CDR1 | CDR2 | CDR3 |
| VH | SYDMH | RIDPQSGGIRYAQKFQG | WDYGGQFDV |
| W33D K59R | (SEQ ID NO: | (SEQ ID NO: 2230) | (SEQ ID NO: |
| Y104Q | 2229) | | 2231) |
| (06BLM) | | | |
| VL | RASQDIYNYLN | YTSILET | QQGDTLPWT |
| T30Y R53I | (SEQ ID NO: | (SEQ ID NO: 2233) | (SEQ ID NO: |
| H55E S56T | 2232) | | 2234) |
| (06BLM) | | | |
| | Chothia numbering scheme | | |
| | CDR1 | CDR2 | CDR3 |
| VH | GYTFTSY | DPQSGG | WDYGGQFDV |
| W33D K59R | (SEQ ID NO: | (SEQ ID NO: 2236) | (SEQ ID NO: |
| Y104Q | 2235) | | 2237) |
| (06BLM) | | | |
| VL | RASQDIYNYLN | YTSILET | QQGDTLPWT |
| T30Y R53I | (SEQ ID NO: | (SEQ ID NO: 2239) | (SEQ ID NO: |
| H55E S56T | 2238) | | 2240) |
| (06BLM) | | | |
| | ABM numbering scheme | | |
| | CDR1 | CDR2 | CDR3 |
| VH | GYTFTSYDMH | RIDPQSGGIR | WDYGGQFDV |
| W33D K59R | (SEQ ID NO: | (SEQ ID NO: 2242) | (SEQ ID NO: |
| Y104Q | 2241) | | 2243) |
| (06BLM) | | | |
| VL | RASQDIYNYLN | YTSILET | QQGDTLPWT |
| T30Y R53I | (SEQ ID NO: | (SEQ ID NO: 2245) | (SEQ ID NO: |
| H55E S56T | 2244) | | 2246) |
| (06BLM) | | | |
| | IMGT numbering scheme | | |
| | CDR1 | CDR2 | CDR3 |
| VH | GYTFTSYD | IDPQSGGI | ARWDYGGQFDV |
| W33D K59R | (SEQ ID NO: | (SEQ ID NO: 2248) | (SEQ ID NO: |
| Y104Q | 2247) | | 2249) |
| (06BLM) | | | |
| VL | QDIYNY | YTS | QQGDTLPWT |
| T30Y R53I | (SEQ ID NO: | (SEQ ID NO: 2251) | (SEQ ID NO: |
| H55E S56T | 2250) | | 2252) |
| (06BLM) | | | |
| | Kabat numbering scheme | | |
| V-region ID | CDR1 | CDR2 | CDR3 |
| VH W33D K59R | SYDIH | RIDPQSGGIRYAQKFQG | WDYGGYFDV |
| (07BLM) | (SEQ ID NO: | (SEQ ID NO: 2254) | (SEQ ID NO: |
| | 2253) | | 2255) |
| VL W33D K59R | RASQDIYSYLN | YTSNLET | QQGDTLPWT |
| (07BLM) | (SEQ ID NO: | (SEQ ID NO: 2257) | (SEQ ID NO: |
| | 2256) | | 2258) |
| | Chothia numbering scheme | | |
| | CDR1 | CDR2 | CDR3 |
| VH W33D K59R | GYTFTSY | DPQSGG | WDYGGYFDV |
| (07BLM) | (SEQ ID NO: | (SEQ ID NO: 2260) | (SEQ ID NO: |
| | 2259) | | 2261) |
| VL W33D K59R | RASQDIYSYLN | YTSNLET | QQGDTLPWT |
| (07BLM) | (SEQ ID NO: | (SEQ ID NO: 2263) | (SEQ ID NO: |
| | 2262) | | 2264) |
| | ABM numbering scheme | | |
| | CDR1 | CDR2 | CDR3 |
| VH W33D K59R | GYTFTSYDMH | RIDPQSGGIR | WDYGGYFDV |
| (07BLM) | (SEQ ID NO: | (SEQ ID NO: 2266) | (SEQ ID NO: |
| | 2265) | | 2267) |
| VL W33D K59R | RASQDIYSYLN | YTSNLET | QQGDTLPWT |
| (07BLM) | (SEQ ID NO: | (SEQ ID NO: 2269) | (SEQ ID NO: |
| | 2268) | | 2270) |
| | IMGT numbering scheme | | |
| | CDR1 | CDR2 | CDR3 |
| VH W33D K59R | GYTFTSYD | IDPQSGGI | ARWDYGGYFDV |
| (07BLM) | (SEQ ID NO: | ((SEQ ID NO: | (SEQ ID NO: |
| | 2271) | 2272) | 2273) |
| VL W33D K59R | QDIYSY | YTS | QQGDTLPWT |
| (07BLM) | (SEQ ID NO: | (SEQ ID NO: 2275) | (SEQ ID NO: |
| | 2274) | | 2276) |
| | Kabat numbering scheme | | |
| V-region ID | CDR1 | CDR2 | CDR3 |
| VH W33D I58T | SYDMH | RIDPQSGGTKYAQKFQG | WDYGGQFDV |
| Y104Q | (SEQ ID NO: | (SEQ ID NO: 2278) | (SEQ ID NO: |
| (08BLM) | 2277) | | 2279) |
| VL T30Y R53G | RASQDIYNYLN | YTSGLET | QQGDTLPWT |
| H55E S56T | (SEQ ID NO: | (SEQ ID NO: 2281) | (SEQ ID NO: |
| (08BLM) | 2280) | | 2282) |
| | Chothia numbering scheme | | |
| | CDR1 | CDR2 | CDR3 |
| VH W33D I58T | GYTFTSY | DPQSGG | WDYGGQFDV |
| Y104Q | (SEQ ID NO: | (SEQ ID NO: 2284) | (SEQ ID NO: |
| (08BLM) | 2283) | | 2285) |
| VL T30Y R53G | RASQDIYNYLN | YTSGLET | QQGDTLPWT |
| H55E S56T | (SEQ ID NO: | | (SEQ ID NO: 2287) |
| (08BLM) | 2286) | | 2288) |
| | ABM numbering scheme | | |
| | CDR1 | CDR2 | CDR3 |
| VH W33D I58T | GYTFTSYDMH | RIDPQSGGTK | WDYGGQFDV |
| Y104Q | (SEQ ID NO: | (SEQ ID NO: 2290) | (SEQ ID NO: |
| (08BLM) | 2289) | | 2291) |
| VL T30Y R53G | RASQDIYNYLN | YTSGLET | QQGDTLPWT |
| H55E S56T | (SEQ ID NO: | (SEQ ID NO: 2293) | (SEQ ID NO: |
| (08BLM) | 2292) | | 2294) |
| | IMGT numbering scheme | | |
| | CDR1 | CDR2 | CDR3 |
| VH W33D I58T | GYTFTSYD | IDPQSGGT | ARWDYGGQFDV |
| Y104Q | (SEQ ID NO: | (SEQ ID NO: 2296) | (SEQ ID NO: |
| (08BLM) | 2295) | | 2297) |
| VL T30Y R53G | QDIYNY | YTS | QQGDTLPWT |
| H55E S56T | (SEQ ID NO: | (SEQ ID NO: 2299) | (SEQ ID NO: |
| (08BLM) | 2298) | | 2300) |
| | Kabat numbering scheme | | |
| V-region ID | CDR1 | CDR2 | CDR3 |
| VH W33D Q54I | SYDMH | RIDPISGGIKYAQKFQG | WDYGGYFDV |
| (09BLM) | (SEQ ID NO: | (SEQ ID NO: 2302) | (SEQ ID NO: |
| | 2301) | | 2303) |
| VL T30F R53H | RASQDIFNYLN | YTSHLET | QQGDTLPWT |
| H55E S56T | (SEQ ID NO: | (SEQ ID NO: 2305) | (SEQ ID NO: |
| (09BLM) | 2304) | | 2306) |
| | Chothia numbering scheme | | |
| | CDR1 | CDR2 | CDR3 |
| VH W33D Q541 | GYTFTSY | DPISGG | WDYGGYFDV |
| (09BLM) | (SEQ ID NO: | (SEQ ID NO: 2308) | (SEQ ID NO: |
| | 2307) | | 2309) |
| VL T30F R53H | RASQDIFNYLN | YTSHLET | QQGDTLPWT |
| H55E S56T | (SEQ ID NO: | (SEQ ID NO: 2311) | (SEQ ID NO: |
| (09BLM) | 2310) | | 2312) |
| | ABM numbering scheme | | |
| | CDR1 | CDR2 | CDR3 |
| VH W33D Q54I | GYTFTSYDMH | RIDPISGGIK | WDYGGYFDV |
| (09BLM) | (SEQ ID NO: | (SEQ ID NO: 2314) | (SEQ ID NO: |
| | 2313) | | 2315) |
| VL T30F R53H | RASQDIFNYLN | YTSHLET | QQGDTLPWT |
| H55E S56T | (SEQ ID NO: | (SEQ ID NO: 2317) | (SEQ ID NO: |
| (09BLM) | 2316) | | 2318) |
| | IMGT numbering scheme | | |
| | CDR1 CDR2 | | CDR3 |
| VH W33D Q54I | GYTFTSYD | IDPISGGI (SEQ ID | ARWDYGGYFDV |
| (09BLM) | (SEQ ID NO: | NO: 2320) | (SEQ ID NO: |
| | 2319) | | 2321) |
| VL T30F R53H | QDIFNY | YTS | QQGDTLPWT |
| H55E S56T | (SEQ ID NO: | (SEQ ID NO: 2323) | (SEQ ID NO: |
| (09BLM) | 2322) | | 2324) |
| | Kabat numbering scheme | | |
| V-region ID | CDR1 | CDR2 | CDR3 |
| VH W33D I58T | SYDMH | RIDPQSGGTKYAQKFQG | WDYGGYFDV |
| (10BLM) | (SEQ ID NO: | (SEQ ID NO: 2326) | (SEQ ID NO: |
| | 2325) | | 2327) |
| VL T30Y R53V | RASQDIYNYLN | YTSVLET | QQGDTLPWT |
| H55E S56T | (SEQ ID NO: | (SEQ ID NO: 2329) | (SEQ ID NO: |
| (10BLM) | 2328) | | 2330) |
| | Chothia numbering scheme | | |
| | CDR1 | CDR2 | CDR3 |
| VH W33D I58T | GYTFTSY | DPQSGG | WDYGGYFDV |
| (10BLM) | (SEQ ID NO: | (SEQ ID NO: 2332) | (SEQ ID NO: |
| | 2331) | | 2333) |
| VL T30Y R53V | RASQDIYNYLN | YTSVLET | QQGDTLPWT |
| H55E S56T | (SEQ ID NO: | (SEQ ID NO: 2335) | (SEQ ID NO: |
| (10BLM) | 2334) | | 2336) |
| | ABM numbering scheme | | |
| | CDR1 | CDR2 | CDR3 |
| VH W33D I58T | GYTFTSYDMH | RIDPQSGGTK | DYGGYFDV |
| (10BLM) | (SEQ ID NO: | (SEQ ID NO: 2338) | (SEQ ID NO: |
| | 2337) | | 2339) |
| VL T30Y R53V | RASQDIYNYLN | YTSVLET | QQGDTLPWT |
| H55E S56T | (SEQ ID NO: | (SEQ ID NO: 2341) | (SEQ ID NO: |
| (10BLM) | 2340) | | 2342) |
| | IMGT numbering scheme | | |
| | CDR1 | CDR2 | CDR3 |
| VH W33D I58T | GYTFTSYD | IDPQSGGT | ARWDYGGYFDV |
| (10BLM) | (SEQ ID NO: | (SEQ ID NO: 2344) | (SEQ ID NO: |
| | 2343) | | 2345) |
| VL T30Y R53V | QDIYNY | YTS | QQGDTLPWT |
| H55E S56T | (SEQ ID NO: | (SEQ ID NO: 2347) | (SEQ ID NO: |
| (10BLM) | 2346) | | 2348) |
| | Kabat numbering scheme | | |
| V-region ID | CDR1 | CDR2 | CDR3 |
| VH W33D | SYDMH | RIDPQSGGIRYAQKFQG | WDYGGYFDV |
| K59R (11BLM) | (SEQ ID NO: | (SEQ ID NO: 2350) | (SEQ ID NO: |
| | 2349) | | 2351) |
| VL T30Y | RASQDIYNYLN | YTSVLET | QQGDTLPWT |
| R53V H55E | (SEQ ID NO: | (SEQ ID NO: 2353) | (SEQ ID NO: |
| S56T (11BLM) | 2352) | | 2354) |
| | Chothia numbering scheme | | |
| | CDR1 | CDR2 | CDR3 |
| VH W33D | GYTFTSY | DPQSGG | WDYGGYFDV |
| K59R (11BLM) | (SEQ ID NO: | (SEQ ID NO: 2356) | (SEQ ID NO: |
| | 2355) | | 2357) |
| VL T30Y | RASQDIYNYLN | YTSVLET | QQGDTLPWT |
| R53V H55E | (SEQ ID NO: | (SEQ ID NO: 2359) | (SEQ ID NO: |
| S56T (11BLM) | 2358) | | 2360) |
| | ABM numbering scheme | | |
| | CDR1 | CDR2 | CDR3 |
| VH W33D | GYTFTSYDMH | RIDPQSGGIR | WDYGGYFDV |
| K59R (11BLM) | (SEQ ID NO: | (SEQ ID NO: 2362) | (SEQ ID NO: |
| | 2361) | | 2363) |
| VL T30Y | RASQDIYNYLN | YTSVLET | QQGDTLPWT |
| R53V H55E | (SEQ ID NO: | (SEQ ID NO: 2365) | (SEQ ID NO: |
| S56T (11BLM) | 2364) | | 2366) |
| | IMGT numbering scheme | | |
| | CDR1 | CDR2 | CDR3 |
| VH W33D | GYTFTSYD | IDPQSGGI | ARWDYGGYFDV |
| K59R (11BLM) | (SEQ ID NO: | (SEQ ID NO: 2368) | (SEQ ID NO: |
| | 2367) | | 2369) |
| VL T30Y | QDIYNY | YTS | QQGDTLPWT |
| R53V H55E | (SEQ ID NO: | (SEQ ID NO: 2371) | (SEQ ID NO: |
| S56T (11BLM) | 2370) | | 2372) |
| | Kabat numbering scheme | | |
| V-region ID | CDR1 | CDR2 | CDR3 |
| VH W33D | SYDMH | RIDPQSGGVKYAQKFQG | WDYGGYFDV |
| I58V (12BLM) | (SEQ ID NO: | (SEQ ID NO: 2374) | (SEQ ID NO: |
| | 2373) | | 2375) |
| VL T30Y R53V | RASQDIYNYLN | YTSVLET | QQGDELPWT |
| T93E H55E | (SEQ ID NO: | (SEQ ID NO: 2377) | (SEQ ID NO: |
| S56T (12BLM) | 2376) | | 2378) |
| | Chothia numbering scheme | | |
| | CDR1 | CDR2 | CDR3 |
| VH W33D | GYTFTSY | DPQSGG | WDYGGYFDV |
| I58V (12BLM) | (SEQ ID NO: | (SEQ ID NO: 2380) | (SEQ ID NO: |
| | 2379) | | 2381) |
| VL T30Y R53V | RASQDIYNYLN | TSVLET | QQGDELPWT |
| T93E H55E | (SEQ ID NO: | (SEQ ID NO: 2383) | (SEQ ID NO: |
| S56T (12BLM) | 2382) | | 2384) |
| | ABM numbering scheme | | |
| | CDR1 | CDR2 | CDR3 |
| VH W33D | GYTFTSYDMH | RIDPQSGGVK | WDYGGYFDV |
| I58V (12BLM) | (SEQ ID NO: | (SEQ ID NO: 2386) | (SEQ ID NO: |
| | 2385) | | 2387) |
| VL T30Y R53V | RASQDIYNYLN | YTSVLET | QQGDELPWT |
| T93E H55E | (SEQ ID NO: | (SEQ ID NO: 2389) | (SEQ ID NO: |
| S56T (12BLM) | 2388) | | 2390) |
| | IMGT numbering scheme | | |
| | CDR1 | CDR2 | CDR3 |
| VH W33D | GYTFTSYD | IDPQSGGV | ARWDYGGYFDV |
| I58V (12BLM) | (SEQ ID NO: | (SEQ ID NO: 2392) | (SEQ ID NO: |
| | 2391) | | 2393) |
| VL T30Y R53V | QDIYNY | YTS | QQGDELPWT |
| T93E H55E | (SEQ ID NO: | (SEQ ID NO: 2395) | (SEQ ID NO: |
| S56T (12BLM) | 2394) | | 2396) |
| | Kabat numbering scheme | | |
| V-region ID | CDR1 | CDR2 | CDR3 |
| VH W33D S55T | SYDMH | RIDPQTGDIKYAQKFQG | WDYGGQFDV |
| G57D Y104Q | (SEQ ID NO: | (SEQ ID NO: 2398) | (SEQ ID NO: |
| (13BLM) | 2397) | | 2399) |
| VL T30Y R53G | RASQDIYNYLN | YTSGLET | QQGDTLPWT |
| H55E S56T | (SEQ ID NO: | (SEQ ID NO: 2401) | (SEQ ID NO: |
| (13BLM) | 2400) | | 2402) |
| | Chothia numbering scheme | | |
| | CDR1 | CDR2 | CDR3 |
| VH W33D S55T | GYTFTSY | DPQTGD | WDYGGQFDV |
| G57D Y104Q | (SEQ ID NO: | (SEQ ID NO: 2404) | (SEQ ID NO: |
| (13BLM) | 2403) | | 2405) |
| VL T30Y R53G | RASQDIYNYLN | YTSGLET | QQGDTLPWT |
| H55E S56T | (SEQ ID NO: | (SEQ ID NO: 2407) | (SEQ ID NO: |
| (13BLM) | 2406) | | 2408) |
| | ABM numbering scheme | | |
| | CDR1 | CDR2 | CDR3 |
| VH W33D S55T | GYTFTSYDMH | RIDPQTGDIK | WDYGGQFDV |
| G57D Y104Q | (SEQ ID NO: | (SEQ ID NO: 2410) | (SEQ ID NO: |
| (13BLM) | 2409) | | 2411) |
| VL T30Y R53G | RASQDIYNYLN | YTSGLET | QQGDTLPWT |
| H55E S56T | (SEQ ID NO: | (SEQ ID NO: 2413) | (SEQ ID NO: |
| (13BLM) | 2412) | | 2414) |
| | IMGT numbering scheme | | |
| | CDR1 | CDR2 | CDR3 |
| VH W33D S55T | GYTFTSYD | IDPQTGDI | ARWDYGGQFDV |
| G57D Y104Q | (SEQ ID NO: | (SEQ ID NO: 2416) | (SEQ ID NO: |
| (13BLM) | 2415) | | 2417) |
| VL T30Y R53G | QDIYNY | YTS | QQGDTLPWT |
| H55E S56T | (SEQ ID NO: | (SEQ ID NO: 2419) | (SEQ ID NO: |
| (13BLM) | 2418) | | 2420) |
| | Kabat numbering scheme | | |
| V-region ID | CDR1 | CDR2 | CDR3 |
| VH W33D M34T | SYDTH | RIDPQSGGIKYAQKFQG | WDYGGYFDV |
| (14BLM) | (SEQ ID NO: | (SEQ ID NO: 2422) | (SEQ ID NO: |
| | 2421) | | 2423) |
| VL T30Y R53Y | RASQDIYNYLN | YTSYLET | QQGDTLPWT |
| H55E S56T | (SEQ ID NO: | (SEQ ID NO: 2425) | (SEQ ID NO : |
| (14BLM) | 2424) | | 2426) |
| | Chothia numbering scheme | | |
| | CDR1 | CDR2 | CDR3 |
| VH W33D M34T | GYTFTSY | DPQSGG | WDYGGYFDV |
| (14BLM) | (SEQ ID NO: | (SEQ ID NO: 2428) | (SEQ ID NO: |
| | 2427) | | 2429) |
| VL T30Y R53Y | RASQDIYNYLN | YTSYLET | QQGDTLPWT |
| H55E S56T | (SEQ ID NO: | (SEQ ID NO: 2431) | (SEQ ID NO: |
| (14BLM) | 2430) | | 2432) |
| | ABM numbering scheme | | |
| | CDR1 | CDR2 | CDR3 |
| VH W33D M34T | GYTFTSYDTH | RIDPQSGGIK | WDYGGYFDV |
| (14BLM) | (SEQ ID NO: | (SEQ ID NO: 2434) | (SEQ ID NO: |
| | 2433) | | 2435) |
| VL T30Y R53Y | RASQDIYNYLN | YTSYLET | QQGDTLPWT |
| H55E S56T | (SEQ ID NO: | (SEQ ID NO: 2437) | (SEQ ID NO: |
| (14BLM) | 2436) | | 2438) |
| | IMGT numbering scheme | | |
| | CDR1 | CDR2 | CDR3 |
| VH W33D M34T | GYTFTSYD | IDPQSGGI | ARWDYGGYFDV |
| (14BLM) | (SEQ ID NO: | (SEQ ID NO: 2440) | (SEQ ID NO: |
| | 2439) | | 2441) |
| VL T30Y R53Y | QDIYNY | YTS | QQGDTLPWT |
| H55E S56T | (SEQ ID NO: | (SEQ ID NO: 2443) | (SEQ ID NO: |
| (14BLM) | 2442) | | 2444) |
| | Kabat numbering scheme | | |
| V-region ID | CDR1 | CDR2 | CDR3 |
| VH W33D | SYDMH | RIDPQSGGIKYAQKFQG | WDYGGYFDV |
| (15BLM) | (SEQ ID NO: | (SEQ ID NO: 2446) | (SEQ ID NO: |
| | 2445) | | 2447) |
| VL T30Y H55E | RASQDIYNYLN | YTSRLET | QQGDTLPWT |
| S56T (15BLM) | (SEQ ID NO: | (SEQ ID NO: 2449) | (SEQ ID NO: |
| | 2448) | | 2450) |
| | Chothia numbering scheme | | |
| | CDR1 | CDR2 | CDR3 |
| VH W33D | GYTFTSY | DPQSGG | WDYGGYFDV |
| (15BLM) | (SEQ ID NO: | (SEQ ID NO: 2452) | (SEQ ID NO: |
| | 2451) | | 2453) |
| VL T30Y H55E | RASQDIYNYLN | YTSRLET | QQGDTLPWT |
| S56T (15BLM) | (SEQ ID NO: | (SEQ ID NO: 2455) | (SEQ ID NO: |
| | 2454) | | 2456) |
| | ABM numbering scheme | | |
| | CDR1 | CDR2 | CDR3 |
| VH W33D | GYTFTSYDMH | RIDPQSGGIK | WDYGGYFDV |
| (15BLM) | (SEQ ID NO: | (SEQ ID NO: 2458) | (SEQ ID NO: |
| | 2457) | | 2459) |
| VL T30Y H55E | RASQDIYNYLN | YTSRLET | QQGDTLPWT |
| S56T (15BLM) | (SEQ ID NO: | (SEQ ID NO: 2461) | (SEQ ID NO: |
| | 2460) | | 2462) |
| | IMGT numbering scheme | | |
| | CDR1 | CDR2 | CDR3 |
| VH W33D | GYTFTSYD | IDPQSGGI | ARWDYGGYFDV |
| (15BLM) | (SEQ ID NO: | (SEQ ID NO: 2464) | (SEQ ID NO: |
| | 2463) | | 2465) |
| VL T30Y H55E | QDIYNY | YTS | QQGDTLPWT |
| S56T (15BLM) | (SEQ ID NO: | (SEQ ID NO: 2467) | (SEQ ID NO: |
| | 2466) | | 2468) |
| | Kabat numbering scheme | | |
| V-region ID | CDR1 | CDR2 | CDR3 |
| VH W33D | SYDMH | RIDPQSGGIKYAQKFQG | WDYGGYFDV |
| (16BLM) | (SEQ ID NO: | (SEQ ID NO: 2470) | (SEQ ID NO: |
| | 2469) | | 2471) |
| VL T30Y R53V | RASQDIYNYLN | YTSVLET | QQGDTLPWT |
| H55E S56T | (SEQ ID NO: | (SEQ ID NO: 2473) | (SEQ ID NO: |
| (16BLM) | 2472) | | 2474 |
| | Chothia numbering scheme | | |
| | CDR1 | CDR2 | CDR3 |
| VH W33D | GYTFTSY | DPQSGG | WDYGGYFDV |
| (16BLM) | (SEQ ID NO: | (SEQ ID NO: 2476) | (SEQ ID NO: |
| | 2475) | | 2477) |
| VL T30Y R53V | RASQDIYNYLN | YTSVLET | QQGDTLPWT |
| H55E S56T | (SEQ ID NO: | (SEQ ID NO: 2479) | (SEQ ID NO: |
| (16BLM) | 2478) | | 2480) |
| | ABM numbering scheme | | |
| | CDR1 | CDR2 | CDR3 |
| VH W33D | GYTFTSYDMH | RIDPQSGGIK | WDYGGYFDV |
| (16BLM) | (SEQ ID NO: | (SEQ ID NO: 2482) | (SEQ ID NO: |
| | 2481) | | 2483) |
| VL T30Y R53V | RASQDIYNYLN | YTSVLET | QQGDTLPWT |
| H55E S56T | (SEQ ID NO: | (SEQ ID NO: 2485) | (SEQ ID NO: |
| (16BLM) | 2484) | | 2486) |
| | IMGT numbering scheme | | |
| | CDR1 | CDR2 | CDR3 |
| VH W33D | GYTFTSYD | IDPQSGGI | ARWDYGGYFDV |
| (16BLM) | (SEQ ID NO: | (SEQ ID NO: 2488) | (SEQ ID NO: |
| | 2487) | | 2489) |
| VL T30Y R53V | QDIYNY | YTS | QQGDTLPWT |
| H55E S56T | (SEQ ID NO: | (SEQ ID NO: 2491) | (SEQ ID NO: |
| (16BLM) | 2490) | | 2492) |
| | Kabat numbering scheme | | |
| V-region ID | CDR1 | CDR2 | CDR3 |
| VH W33D | SYDMH | RIDPQSGGIKYAQKFQG | WDYGGYFDV |
| (017BLM) | (SEQ ID NO: | (SEQ ID NO: 2494) | (SEQ ID NO: |
| | 2493) | | 2495) |
| VL T30Y T93E | RASQDIYNYLN | YTSRLET | QQGDELPWT |
| H55E S56T | (SEQ ID NO: | (SEQ ID NO: 2497) | (SEQ ID NO: |
| (017BLM) | 2496) | | 2498) |
| | Chothia numbering scheme | | |
| | CDR1 | CDR2 | CDR3 |
| VH W33D | GYTFTSY | DPQSGG | WDYGGYFDV |
| (017BLM) | (SEQ ID NO: | (SEQ ID NO: 2500) | (SEQ ID NO: |
| | 2499) | | 2501) |
| VL T30Y T93E | RASQDIYNYLN | YTSRLET | QQGDELPWT |
| H55E S56T | (SEQ ID NO: | (SEQ ID NO: 2503) | (SEQ ID NO: |
| (017BLM) | 2502) | | 2504) |
| | ABM numbering scheme | | |
| | CDR1 | CDR2 | CDR3 |
| VH W33D | GYTFTSYDMH | RIDPQSGGIK | WDYGGYFDV |
| (017BLM) | (SEQ ID NO: | (SEQ ID NO: 2506) | (SEQ ID NO: |
| | 2505) | | 2507) |
| VL T30Y T93E | RASQDIYNYLN | YTSRLET | QQGDELPWT |
| H55E S56T | (SEQ ID NO: | (SEQ ID NO: 2509) | (SEQ ID NO: |
| (017BLM) | 2508) | | 2510) |
| | IMGT numbering scheme | | |
| | CDR1 | CDR2 | CDR3 |
| VH W33D | GYTFTSYD | IDPQSGGI | ARWDYGGYFDV |
| (017BLM) | (SEQ ID NO: | (SEQ ID NO: 2512) | (SEQ ID NO: |
| | 2511) | | 2513) |
| VL T30Y T93E | QDIYNY | YTS | QQGDELPWT |
| H55E S56T | (SEQ ID NO: | (SEQ ID NO: 2515) | (SEQ ID NO: |
| (017BLM) | 2514) | | 2516) |
| | Kabat numbering scheme | | |
| V-region ID | CDR1 | CDR2 | CDR3 |
| VH W33D W99Y | SYDMH | RIDPQSGGIKYAQKFQG | YDHGGYFDV |
| Y101H (18BLM) | (SEQ ID NO: | (SEQ ID NO: 2518) | (SEQ ID NO: |
| | 2517) | | 2519) |
| VL T30Y H55E | RASQDIYNYLN | YTSRLET | QQGDTLPWT |
| S56T (18BLM) | (SEQ ID NO: | (SEQ ID NO: 2521) | (SEQ ID NO: |
| | 2520) | | 2522) |
| | Chothia numbering scheme | | |
| | CDR1 | CDR2 | CDR3 |
| VH W33D W99Y | GYTFTSY | DPQSGG | YDHGGYFDV |
| Y101H (18BLM) | (SEQ ID NO: | (SEQ ID NO: 2524) | (SEQ ID NO: |
| | 2523) | | 2525) |
| VL T30Y H55E | RASQDIYNYLN | YTSRLET | QQGDTLPWT |
| S56T (18BLM) | (SEQ ID NO: | (SEQ ID NO: 2527) | (SEQ ID NO: |
| | 2526) | | 2528) |
| | ABM numbering scheme | | |
| | CDR1 | CDR2 | CDR3 |
| VH W33D W99Y | GYTFTSYDMH | RIDPQSGGIK | YDHGGYFDV |
| Y101H (18BLM) | (SEQ ID NO: | (SEQ ID NO: 2530) | (SEQ ID NO: |
| | 2529) | | 2531) |
| VL T30Y H55E | RASQDIYNYLN | YTSRLET | QQGDTLPWT |
| S56T (18BLM) | (SEQ ID NO: | (SEQ ID NO: 2533) | (SEQ ID NO: |
| | 2532) | | 2534) |
| | IMGT numbering scheme | | |
| | CDR1 | CDR2 | CDR3 |
| VH W33D W99Y | GYTFTSYD | IDPQSGGI | ARYDHGGYFDV |
| Y101H (18BLM) | (SEQ ID NO: | (SEQ ID NO: 2536) | (SEQ ID NO: |
| | 2535) | | 2537) |
| VL T30Y H55E | QDIYNY | YTS | QQGDTLPWT |
| S56T (18BLM) | (SEQ ID NO: | (SEQ ID NO: 2539) | (SEQ ID NO: |
| | 2538) | | 2540) |
| | Kabat numbering scheme | | |
| V-region ID | CDR1 | CDR2 | CDR3 |
| VH W33D W99Y | SYDMH | RIDPQSGGIKYAQKFQG | YDHGGYFDV |
| Y101H (19BLM) | (SEQ ID NO: | (SEQ ID NO: 2542) | (SEQ ID NO: |
| | 2541) | | 2543) |
| VL T30Y R53Y | RASQDIYNYLN | YTSYLET | QQGDTLPWT |
| H55E S56T | (SEQ ID NO: | (SEQ ID NO: 2545) | (SEQ ID NO: |
| (19BLM) | 2544) | | 2546) |
| | Chothia numbering scheme | | |
| | CDR1 | CDR2 | CDR3 |
| VH W33D W99Y | GYTFTSY | DPQSGG | YDHGGYFDV |
| Y101H (19BLM) | (SEQ ID NO: | (SEQ ID NO: 2548) | (SEQ ID NO: |
| | 2547) | | 2549) |
| VL T30Y R53Y | RASQDIYNYLN | YTSYLET | QQGDTLPWT |
| H55E S56T | (SEQ ID NO: | (SEQ ID NO: 2551) | (SEQ ID NO: |
| (19BLM) | 2550) | | 2552) |
| | ABM numbering scheme | | |
| | CDR1 | CDR2 | CDR3 |
| VH W33D W99Y | GYTFTSYDMH | RIDPQSGGIK | YDHGGYFDV |
| Y101H (19BLM) | (SEQ ID NO: | (SEQ ID NO: 2554) | (SEQ ID NO: |
| | 2553) | | 2555) |
| VL T30Y R53Y | RASQDIYNYLN | YTSYLET | QQGDTLPWT |
| H55E S56T | (SEQ ID NO: | (SEQ ID NO: 2557) | (SEQ ID NO: |
| (19BLM) | 2556) | | 2558) |
| | IMGT numbering scheme | | |
| | CDR1 | CDR2 | CDR3 |
| VH W33D W99Y | GYTFTSYD | IDPQSGGI | ARYDHGGYFDV |
| Y101H (19BLM) | (SEQ ID NO: | (SEQ ID NO: 2560) | (SEQ ID NO: |
| | 2559) | | 2561) |
| VL T30Y R53Y | QDIYNY | YTS | QQGDTLPWT |
| H55E S56T | (SEQ ID NO: | (SEQ ID NO: 2563) | (SEQ ID NO: |
| (19BLM) | 2562) | | 2564) |
| | Kabat numbering scheme | | |
| V-region ID | CDR1 | CDR2 | CDR3 |
| VH W33D I58E | SYDMH | RIDPQSGGEKYAQKFQG | YDYGGYFDV |
| W99Y (20BLM) | (SEQ ID NO: | (SEQ ID NO: 2566) | (SEQ ID NO: |
| | 2565) | | 2567) |
| VL T30Y H55E | RASQDIYNYLN | YTSRLET | QQGDTLPWT |
| S56T (20BLM) | (SEQ ID NO: | (SEQ ID NO: 2569) | (SEQ ID NO: |
| | 2568) | | 2570) |
| | Chothia numbering scheme | | |
| | CDR1 | CDR2 | CDR3 |
| VH W33D I58E | GYTFTSY | DPQSGG | YDYGGYFDV |
| W99Y (20BLM) | (SEQ ID NO: | (SEQ ID NO: 2572) | (SEQ ID NO: |
| | 2571) | | 2573) |
| VL T30Y H55E | RASQDIYNYLN | YTSRLET | QQGDTLPWT |
| S56T (20BLM) | (SEQ ID NO: | (SEQ ID NO: 2575) | (SEQ ID NO: |
| | 2574) | | 2576) |
| | ABM numbering scheme | | |
| | CDR1 | CDR2 | CDR3 |
| VH W33D I58E | GYTFTSYDMH | RIDPQSGGEK | YDYGGYFDV |
| W99Y (20BLM) | (SEQ ID NO: | (SEQ ID NO: 2578) | (SEQ ID NO: |
| | 2577) | | 2579) |
| VL T30Y H55E | RASQDIYNYLN | YTSRLET | QQGDTLPWT |
| S56T (20BLM) | (SEQ ID NO: | (SEQ ID NO: 2581) | (SEQ ID NO: |
| | 2580) | | 2582) |
| | IMGT numbering scheme | | |
| | CDR1 | CDR2 | CDR3 |
| VH W33D I58E | GYTFTSYD | IDPQSGGE | ARYDYGGYFDV |
| W99Y (20BLM) | (SEQ ID NO: | (SEQ ID NO: 2584) | (SEQ ID NO: |
| | 2583) | | 2585) |
| VL T30Y H55E | QDIYNY | YTS | QQGDTLPWT |
| S56T (20BLM) | (SEQ ID NO: | (SEQ ID NO: 2587) | (SEQ ID NO: |
| | 2586) | | 2588) |

TABLE 45B
VH and VL amino acid sequences for the 15 selected affinity maturated humanized antibodies

| Lot ID & | | VH | | VL |
|---|---|---|---|---|
| Affinity | VH amino acid sequence* | Mutations | VL amino acid sequence** | Mutations |
| 06BLM | EVQLVQSGAEVKKPGASVKVSCKASGYTFT | W33D | DIQMTQSPSSLSASVGDRVTITCRASQDIYNYLNWYQQKPG | T30Y |
| | SYDMHWVRQAPGQGLEWMGRIDPQSGGIRY | K59R | KAVKLLIYYTSILETGVPSRFSGSGSGTDYTLTISSLQPED | R53I |
| | AQKFQGRATLTVDTSTSTAYMELSRLRSDD | Y104Q | FATYFCQQGDTLPWTFGQGTKLEIK | H55E |
| | TAVYYCARWDYGGQFDVWGQGTLVTVSS | | (SEQ ID NO: 2604) | S56T |
| | (SEQ ID NO: 2589) | | | |
| 07BLM | EVQLVQSGAEVKKPGASVKVSCKASGYTFT | W33D | DIQMTQSPSSLSASVGDRVTITCRASQDIYSYLNWYQQKPG | T30Y |
| | SYDMHWVRQAPGQGLEWMGRIDPQSGGIRY | K59R | KAVKLLIYYTSNLETGVPSRFSGSGSGTDYTLTISSLQPED | N31S |
| | AQKFQGRATLTVDTSTSTAYMELSRLRSDD | | FATYFCQQGDTLPWTFGQGTKLEIK | R53N |
| | TAVYYCARWDYGGYFDVWGQGTLVTVSS | | (SEQ ID NO: 2605) | H55E |
| | (SEQ ID NO: 2590) | | | S56T |
| 08BLM | EVQLVQSGAEVKKPGASVKVSCKASGYTFT | W33D | DIQMTQSPSSLSASVGDRVTITCRASQDIYNYLNWYQQKPG | T30Y |
| | SYDMHWVRQAPGQGLEWMGRIDPQSGGTKY | I58T | KAVKLLIYYTSGLETGVPSRFSGSGSGTDYTLTISSLQPED | R53G |
| | AQKFQGRATLTVDTSTSTAYMELSRLRSDD | Y104Q | FATYFCQQGDTLPWTFGQGTKLEIK | H55E |
| | TAVYYCARWDYGGQFDVWGQGTLVTVSS | | (SEQ ID NO: 2606) | S56T |
| | (SEQ ID NO: 2591) | | | |
| 09BLM | EVQLVQSGAEVKKPGASVKVSCKASGYTFT | W33D | DIQMTQSPSSLSASVGDRVTITCRASQDIFNYLNWYQQKPG | T30F |
| | SYDMHWVRQAPGQGLEWMGRIDPISGGIKY | Q54I | KAVKLLIYYTSHLETGVPSRFSGSGSGTDYTLTISSLQPED | R53H |
| | AQKFQGRATLTVDTSTSTAYMELSRLRSDD | | FATYFCQQGDTLPWTFGQGTKLEIK | H55E |
| | TAVYYCARWDYGGYFDVWGQGTLVTVSS | | (SEQ ID NO: 2607) | S56T |
| | (SEQ ID NO: 2592) | | | |
| 10BLM | EVQLVQSGAEVKKPGASVKVSCKASGYTFT | W33D | DIQMTQSPSSLSASVGDRVTITCRASQDIYNYLNWYQQKPG | T30Y |
| | SYDMHWVRQAPGQGLEWMGRIDPQSGGTKY | I58T | KAVKLLIYYTSVLETGVPSRFSGSGSGTDYTLTISSLQPED | R53V |
| | AQKFQGRATLTVDTSTSTAYMELSRLRSDD | | FATYFCQQGDTLPWTFGQGTKLEIK | H55E |
| | TAVYYCARWDYGGYFDVWGQGTLVTVSS | | (SEQ ID NO: 2608) | S56T |
| | (SEQ ID NO: 2593) | | | |
| 11BLM | EVQLVQSGAEVKKPGASVKVSCKASGYTFT | W33D | DIQMTQSPSSLSASVGDRVTITCRASQDIYNYLNWYQQKPG | T30Y |
| | SYDMHWVRQAPGQGLEWMGRIDPQSGGIRY | K59R | KAVKLLIYYTSVLETGVPSRFSGSGSGTDYTLTISSLQPED | R53V |
| | AQKFQGRATLTVDTSTSTAYMELSRLRSDD | | FATYFCQQGDTLPWTFGQGTKLEIK | H55E |
| | TAVYYCARWDYGGYFDVWGQGTLVTVSS | | (SEQ ID NO: 2609) | S56T |
| | (SEQ ID NO: 2594) | | | |
| 12BLM | EVQLVQSGAEVKKPGASVKVSCKASGYTFT | W33D | DIQMTQSPSSLSASVGDRVTITCRASQDIYNYLNWYQQKPG | T30Y |
| | SYDMHWVRQAPGQGLEWMGRIDPQSGGVKY | I58V | KAVKLLIYYTSVLETGVPSRFSGSGSGTDYTLTISSLQPED | R53V |
| | AQKFQGRATLTVDTSTSTAYMELSRLRSDD | | FATYFCQQGDELPWTFGQGTKLEIK | T93E |
| | TAVYYCARWDYGGYFDVWGQGTLVTVSS | | (SEQ ID NO: 2610) | H55E |
| | (SEQ ID NO: 2595) | | | S56T |
| 13BLM | EVQLVQSGAEVKKPGASVKVSCKASGYTFT | W33D | DIQMTQSPSSLSASVGDRVTITCRASQDIYNYLNWYQQKPG | T30Y |
| | SYDMHWVRQAPGQGLEWMGRIDPQTGDIKY | S55T | KAVKLLIYYTSGLETGVPSRFSGSGSGTDYTLTISSLQPED | R53G |
| | AQKFQGRATLTVDTSTSTAYMELSRLRSDD | G57D | FATYFCQQGDTLPWTFGQGTKLEIK | H55E |
| | TAVYYCARWDYGGQFDVWGQGTLVTVSS | Y104Q | (SEQ ID NO: 2611) | S56T |
| | (SEQ ID NO: 2596) | | | |
| 14BLM | EVQLVQSGAEVKKPGASVKVSCKASGYTFT | W33D | DIQMTQSPSSLSASVGDRVTITCRASQDIYNYLNWYQQKPG | T30Y |
| | SYDTHWVRQAPGQGLEWMGRIDPQSGGIKY | M34T | KAVKLLIYYTSYLETGVPSRFSGSGSGTDYTLTISSLQPED | R53Y |
| | AQKFQGRATLTVDTSTSTAYMELSRLRSDD | | FATYFCQQGDTLPWTFGQGTKLEIK | H55E |
| | TAVYYCARWDYGGYFDVWGQGTLVTVSS | | (SEQ ID NO: 2612) | S56T |
| | (SEQ ID NO: 2597) | | | |
| 15BLM | EVQLVQSGAEVKKPGASVKVSCKASGYTFT | W33D | DIQMTQSPSSLSASVGDRVTITCRASQDIYNYLNWYQQKPG | T30Y |
| | SYDMHWVRQAPGQGLEWMGRIDPQSGGIKY | | KAVKLLIYYTSRLETGVPSRFSGSGSGTDYTLTISSLQPED | H55E |
| | AQKFQGRATLTVDTSTSTAYMELSRLRSDD | | FATYFCQQGDTLPWTFGQGTKLEIK | S56T |
| | TAVYYCARWDYGGYFDVWGQGTLVTVSS | | (SEQ ID NO: 2613) | |
| | (SEQ ID NO: 2598) | | | |
| 16BLM | EVQLVQSGAEVKKPGASVKVSCKASGYTFT | W33D | DIQMTQSPSSLSASVGDRVTITCRASQDIYNYLNWYQQKPG | T30Y |
| | SYDMHWVRQAPGQGLEWMGRIDPQSGGIKY | | KAVKLLIYYTSVLETGVPSRFSGSGSGTDYTLTISSLQPED | R53V |
| | AQKFQGRATLTVDTSTSTAYMELSRLRSDD | | FATYFCQQGDTLPWTFGQGTKLEIK | H55E |
| | TAVYYCARWDYGGYFDVWGQGTLVTVSS | | (SEQ ID NO: 2614) | S56T |
| | (SEQ ID NO: 2599) | | | |
| 17BLM | EVQLVQSGAEVKKPGASVKVSCKASGYTFT | W33D | DIQMTQSPSSLSASVGDRVTITCRASQDIYNYLNWYQQKPG | T30Y |
| | SYDMHWVRQAPGQGLEWMGRIDPQSGGIKY | | KAVKLLIYYTSRLETGVPSRFSGSGSGTDYTLTISSLQPED | T93E |
| | AQKFQGRATLTVDTSTSTAYMELSRLRSDD | | FATYFCQQGDELPWTFGQGTKLEIK | H55E |
| | TAVYYCARWDYGGYFDVWGQGTLVTVSS | | (SEQ ID NO: 2615) | S56T |
| | (SEQ ID NO: 2600) | | | |
| 18BLM | EVQLVQSGAEVKKPGASVKVSCKASGYTFT | W33D | DIQMTQSPSSLSASVGDRVTITCRASQDIYNYLNWYQQKPG | T30Y |
| | SYDMHWVRQAPGQGLEWMGRIDPQSGGIKY | W99Y | KAVKLLIYYTSRLETGVPSRFSGSGSGTDYTLTISSLQPED | H55E |
| | AQKFQGRATLTVDTSTSTAYMELSRLRSDD | Y101H | FATYFCQQGDTLPWTFGQGTKLEIK | S56T |
| | TAVYYCARYDHGGYFDVWGQGTLVTVSS | | (SEQ ID NO: 2616) | |
| | (SEQ ID NO: 2601) | | | |
| 19BLM | EVQLVQSGAEVKKPGASVKVSCKASGYTFT | W33D | DIQMTQSPSSLSASVGDRVTITCRASQDIYNYLNWYQQKPG | T30Y |
| | SYDMHWVRQAPGQGLEWMGRIDPQSGGIKY | W99Y | KAVKLLIYYTSYLETGVPSRFSGSGSGTDYTLTISSLQPED | R53Y |
| | AQKFQGRATLTVDTSTSTAYMELSRLRSDD | Y101H | FATYFCQQGDTLPWTFGQGTKLEIK | H55E |
| | TAVYYCARYDHGGYFDVWGQGTLVTVSS | | (SEQ ID NO: 2617) | S56T |
| | (SEQ ID NO: 2602) | | | |
| 20BLM | EVQLVQSGAEVKKPGASVKVSCKASGYTFT | W33D | DIQMTQSPSSLSASVGDRVTITCRASQDIYNYLNWYQQKPG | S56T |
| | SYDMHWVRQAPGQGLEWMGRIDPQSGGEKY | I58E | KAVKLLIYYTSRLETGVPSRFSGSGSGTDYTLTISSLQPED | T30Y |
| | AQKFQGRATLTVDTSTSTAYMELSRLRSDD | W99Y | FATYFCQQGDTLPWTFGQGTKLEIK | H55E |
| | TAVYYCARYDYGGYFDVWGQGTLVTVSSAS | | (SEQ ID NO: 2618) | |
| | (SEQ ID NO: 2603) | | | |

TABLE 45C
Amino acid sequences for the 15 selected affinity maturation antibody clone heavy chains and light
chains

| Lot ID | | | VH | | VL |
|---|---|---|---|---|---|
| & | | | Muta- | | Muta- |
| Affinity | ID | Heavy chain amino acid sequence* | tions | Light chain amino acid sequence ** | tions |
| 06BLM | 12hr- | EVQLVQSGAEVKKPGASVKVSCKASGYTFTSYDM | W33D | DIQMTQSPSSLSASVGDRVTITCRASQDIYNYLNWYQQK | T30Y |
| (4 | P1-7 | HWVRQAPGQGLEWMGRIDPQSGGIRYAQKFQGRA | K59R | PGKAVKLLIYYTSILETGVPSRFSGSGSGTDYTLTISSL | R53I |
| pM) | | TLTVDTSTSTAYMELSRLRSDDTAVYYCARWDYG | Y104Q | QPEDFATYFCQQGDTLPWTFGQGTKLEIK | H55E |
| | | GQFDVWGQGTLVTVSSASTKGPSVFPLAPSSKST | | (SEQ ID NO: 2634) | S56T |
| | | SGGTAALGCLVKDYFPEPVTVSWNSGALTSGVHT | | | |
| | | FPAVLQSSGLYSLSSVVTVPSSSLGTQTYICNVN | | | |
| | | HKPSNTKVDKKVEPKSCDKTHTCPPCPAPEaaGG | | | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2619) | | | |
| 07BLM | 12hr- | EVQLVQSGAEVKKPGASVKVSCKASGYTFTSYDM | W33D | DIQMTQSPSSLSASVGDRVTITCRASQDIYSYLNWYQQK | T30Y |
| (59 | P1-13 | HWVRQAPGQGLEWMGRIDPQSGGIRYAQKFQGRA | K59R | PGKAVKLLIYYTSNLETGVPSRFSGSGSGTDYTLTISSL | N31S |
| pM) | | TLTVDTSTSTAYMELSRLRSDDTAVYYCARWDYG | | QPEDFATYFCQQGDTLPWTFGQGTKLEIKRTVAAPSVFI | R53N |
| | | GYFDVWGQGTLVTVSSASTKGPSVFPLAPSSKST | | FPPSDEQLKSGTASVVCLLNNFYPREAKVQWKVDNALQS | H55E |
| | | SGGTAALGCLVKDYFPEPVTVSWNSGALTSGVHT | | GNSQESVTEQDSKDSTYSLSSTLTLSKADYEKHKVYACE | S56T |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2635) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2620) | | | |
| 08BLM | 12hr- | EVQLVQSGAEVKKPGASVKVSCKASGYTFTSYDM | W33D | DIQMTQSPSSLSASVGDRVTITCRASQDIYNYLNWYQQK | T30Y |
| 16 | P1-45 | HWVRQAPGQGLEWMGRIDPQSGGTKYAQKFQGRA | I58T | PGKAVKLLIYYTSGLETGVPSRFSGSGSGTDYTLTISSL | R53G |
| pM) | | TLTVDTSTSTAYMELSRLRSDDTAVYYCARWDYG | Y104Q | QPEDFATYFCQQGDTLPWTFGQGTKLEIKRTVAAPSVFI | H55E |
| | | GQFDVWGQGTLVTVSSASTKGPSVFPLAPSSKST | | FPPSDEQLKSGTASVVCLLNNFYPREAKVQWKVDNALQS | S56T |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2636) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2621) | | | |
| 09BLM | 12hr- | EVQLVQSGAEVKKPGASVKVSCKASGYTFTSYDM | W33D | DIQMTQSPSSLSASVGDRVTITCRASQDIFNYLNWYQQK | T30F |
| (81 | P1-34 | HWVRQAPGOGLEWMGRIDPISGGIKYAQKFQGRA | Q54I | PGKAVKLLIYYTSHLETGVPSRFSGSGSGTDYTLTISSL | R53H |
| pM) | | TLTVDTSTSTAYMELSRLRSDDTAVYYCARWDYG | | QPEDFATYFCQQGDTLPWTFGQGTKLEIKRTVAAPSVFI | H55E |
| | | GYFDVWGQGTLVTVSSASTKGPSVFPLAPSSKST | | FPPSDEQLKSGTASVVCLLNNFYPREAKVQWKVDNALQS | S56T |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2637) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2622) | | | |
| 10BLM | 2hr- | EVQLVQSGAEVKKPGASVKVSCKASGYTFTSYDM | W33D | DIQMTQSPSSLSASVGDRVTITCRASQDIYNYLNWYQQK | T30Y |
| (53 | P1-8 | HWVRQAPGQGLEWMGRIDPQSGGTKYAQKFQGRA | I58T | PGKAVKLLIYYTSVLETGVPSRFSGSGSGTDYTLTISSL | R53V |
| pM) | | TLTVDTSTSTAYMELSRLRSDDTAVYYCARWDYG | | QPEDFATYFCQQGDTLPWTFGQGTKLEIKRTVAAPSVFI | H55E |
| | | GYFDVWGQGTLVTVSSASTKGPSVFPLAPSSKST | | FPPSDEQLKSGTASVVCLLNNFYPREAKVQWKVDNALQS | S56T |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2638) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2623) | | | |
| 11BLM | 2hr- | EVQLVQSGAEVKKPGASVKVSCKASGYTFTSYDM | W33D | DIQMTQSPSSLSASVGDRVTITCRASQDIYNYLNWYQQK | T30Y |
| (66 | P1-32 | HWVRQAPGQGLEWMGRIDPQSGGIRYAQKFQGRA | K59R | PGKAVKLLIYYTSVLETGVPSRFSGSGSGTDYTLTISSL | R53V |
| pM) | | TLTVDTSTSTAYMELSRLRSDDTAVYYCARWDYG | | QPEDFATYFCQQGDTLPWTFGQGTKLEIKRTVAAPSVFI | H55E |
| | | GYFDVWGQGTLVTVSSASTKGPSVFPLAPSSKST | | FPPSDEQLKSGTASVVCLLNNFYPREAKVQWKVDNALQS | S56T |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2639) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2624) | | | |
| 12BLM | 2hr- | EVQLVQSGAEVKKPGASVKVSCKASGYTFTSYDM | W33D | DIQMTQSPSSLSASVGDRVTITCRASQDIYNYLNWYQQK | T30Y |
| (24 | P1-47 | HWVRQAPGQGLEWMGRIDPQSGGVKYAQKFQGRA | I58V | PGKAVKLLIYYTSVLETGVPSRFSGSGSGTDYTLTISSL | R53V |
| PM) | | TLTVDTSTSTAYMELSRLRSDDTAVYYCARWDYG | | QPEDFATYFCQQGDELPWTFGQGTKLEIKRTVAAPSVFI | T93E |
| | | GYFDVWGQGTLVTVSSASTKGPSVFPLAPSSKST | | FPPSDEQLKSGTASVVCLLNNFYPREAKVQWKVDNALQS | H55E |
| | | SGGTAALGCLVKDYFPEPVTVSWNSGALTSGVHT | | GNSQESVTEQDSKDSTYSLSSTLTLSKADYEKHKVYACE | S56T |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2640) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2625) | | | |
| 13BLM | 2hr- | EVQLVQSGAEVKKPGASVKVSCKASGYTFTSYDM | W33D | DIQMTQSPSSLSASVGDRVTITCRASQDIYNYLNWYQQK | T30Y |
| (13 | P1-54 | HWVRQAPGQGLEWMGRIDPQTGDIKYAQKFQGRA | G57D | PGKAVKLLIYYTSGLETGVPSRFSGSGSGTDYTLTISSL | R53G |
| pM) | | TLTVDTSTSTAYMELSRLRSDDTAVYYCARWDYG | Y104Q | QPEDFATYFCQQGDTLPWTFGQGTKLEIKRTVAAPSVFI | H55E |
| | | GQFDVWGQGTLVTVSSASTKGPSVFPLAPSSKST | S55T | FPPSDEQLKSGTASVVCLLNNFYPREAKVQWKVDNALQS | S56T |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2641) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2626) | | | |
| 14BLM | 2hr- | EVQLVQSGAEVKKPGASVKVSCKASGYTFTSYDT | W33D | DIQMTQSPSSLSASVGDRVTITCRASQDIYNYLNWYQQK | T30Y |
| (264 | P1-84 | HWVRQAPGQGLEWMGRIDPQSGGIKYAQKFQGRA | M34T | PGKAVKLLIYYTSYLETGVPSRFSGSGSGTDYTLTISSL | R53Y |
| pM) | | TLTVDTSTSTAYMELSRLRSDDTAVYYCARWDYG | | QPEDFATYFCQQGDTLPWTFGQGTKLEIKRTVAAPSVFI | H55E |
| | | GYFDVWGQGTLVTVSSASTKGPSVFPLAPSSKST | | FPPSDEQLKSGTASVVCLLNNFYPREAKVQWKVDNALQS | S56T |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2642) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2627) | | | |
| 15BLM | 2hr- | EVQLVQSGAEVKKPGASVKVSCKASGYTFTSYDM | W33D | DIQMTQSPSSLSASVGDRVTITCRASQDIYNYLNWYQQK | T30Y |
| (297 | P1-85 | HWVRQAPGQGLEWMGRIDPQSGGIKYAQKFQGRA | | PGKAVKLLIYYTSRLETGVPSRFSGSGSGTDYTLTISSL | H55E |
| pM) | | TLTVDTSTSTAYMELSRLRSDDTAVYYCARWDYG | | QPEDFATYFCQQGDTLPWTFGQGTKLEIKRTVAAPSVFI | S56T |
| | | GYFDVWGQGTLVTVSSASTKGPSVFPLAPSSKST | | FPPSDEQLKSGTASVVCLLNNFYPREAKVQWKVDNALQS | |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2643) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2628) | | | |
| 16BLM | 2hr- | EVQLVQSGAEVKKPGASVKVSCKASGYTFTSYDM | W33D | DIQMTQSPSSLSASVGDRVTITCRASQDIYNYLNWYQQK | T30Y |
| (77 | P2-94 | HWVRQAPGOGLEWMGRIDPQSGGIKYAQKFQGRA | | PGKAVKLLIYYTSVLETGVPSRFSGSGSGTDYTLTISSL | R53V |
| pM) | | TLTVDTSTSTAYMELSRLRSDDTAVYYCARWDYG | | QPEDFATYFCQQGDTLPWTFGQGTKLEIKRTVAAPSVFI | H55E |
| | | GYFDVWGQGTLVTVSSASTKGPSVFPLAPSSKST | | FPPSDEQLKSGTASVVCLLNNFYPREAKVQWKVDNALQS | S56T |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2644) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2629) | | | |
| 17BLM | 2hr- | EVQLVQSGAEVKKPGASVKVSCKASGYTFTSYDM | W33D | DIQMTQSPSSLSASVGDRVTITCRASQDIYNYLNWYQQK | T30Y |
| (146 | P2-95 | HWVRQAPGQGLEWMGRIDPQSGGIKYAQKFQGRA | | PGKAVKLLIYYTSRLETGVPSRFSGSGSGTDYTLTISSL | T93E |
| pM) | | TLTVDTSTSTAYMELSRLRSDDTAVYYCARWDYG | | QPEDFATYFCQQGDELPWTFGQGTKLEIKRTVAAPSVFI | H55E |
| | | GYFDVWGQGTLVTVSSASTKGPSVFPLAPSSKST | | FPPSDEQLKSGTASVVCLLNNFYPREAKVQWKVDNALQS | S56T |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2645) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2630) | | | |
| 18BLM | 2hr- | EVQLVQSGAEVKKPGASVKVSCKASGYTFTSYDM | W33D | DIQMTQSPSSLSASVGDRVTITCRASQDIYNYLNWYQQK | T30Y |
| (3737 | P2-11 | HWVRQAPGQGLEWMGRIDPQSGGIKYAQKFQGRA | W99Y | PGKAVKLLIYYTSRLETGVPSRFSGSGSGTDYTLTISSL | H55E |
| pM) | | TLTVDTSTSTAYMELSRLRSDDTAVYYCARYDHG | Y101H | QPEDFATYFCQQGDTLPWTFGQGTKLEIKRTVAAPSVFI | S56T |
| | | GYFDVWGQGTLVTVSSASTKGPSVFPLAPSSKST | | FPPSDEQLKSGTASVVCLLNNFYPREAKVQWKVDNALQS | |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2646) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2631) | | | |
| 19BLM | 2hr- | EVQLVQSGAEVKKPGASVKVSCKASGYTFTSYDM | W33D | DIQMTQSPSSLSASVGDRVTITCRASQDIYNYLNWYQQK | T30Y |
| (961 | P3-16 | HWVRQAPGQGLEWMGRIDPQSGGIKYAQKFQGRA | W99Y | PGKAVKLLIYYTSYLETGVPSRFSGSGSGTDYTLTISSL | R53Y |
| pM) | | TLTVDTSTSTAYMELSRLRSDDTAVYYCARYDHG | Y101H | QPEDFATYFCQQGDTLPWTFGQGTKLEIKRTVAAPSVFI | H55E |
| | | GYFDVWGQGTLVTVSSASTKGPSVFPLAPSSKST | | FPPSDEQLKSGTASVVCLLNNFYPREAKVQWKVDNALQS | S56T |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2647) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2632) | | | |
| 20BLM | 2hr- | EVQLVQSGAEVKKPGASVKVSCKASGYTFTSYDM | W33D | DIQMTQSPSSLSASVGDRVTITCRASQDIYNYLNWYQQK | T30Y |
| (438 | P3-33 | HWVRQAPGQGLEWMGRIDPQSGGEKYAQKFQGRA | I58E | PGKAVKLLIYYTSRLETGVPSRFSGSGSGTDYTLTISSL | H55E |
| pM) | | TLTVDTSTSTAYMELSRLRSDDTAVYYCARYDYG | W99Y | QPEDFATYFCQQGDTLPWTFGQGTKLEIKRTVAAPSVFI | S56T |
| | | GYFDVWGQGTLVTVSSASTKGPSVFPLAPSSKST | | FPPSDEQLKSGTASVVCLLNNFYPREAKVQWKVDNALQS | |
| | | SGGTAALGCLVKDYFPEPVTVSWNSGALTSGVHT | | GNSQESVTEQDSKDSTYSLSSTLTLSKADYEKHKVYACE | |
| | | FPAVLQSSGLYSLSSVVTVPSSSLGTQTYICNVN | | VTHQGLSSPVTKSFNRGEC | |
| | | HKPSNTKVDKKVEPKSCDKTHTCPPCPAPEaaGG | | (SEQ ID NO: 2648) | |
| | | PSVFLFPPKPKDTLMISRTPEVTCVVVsVSHEDP | | | |
| | | EVKFNWYVDGVEVHNAKTKPREEQYNSTYRVVSV | | | |
| | | LTVLHQDWLNGKEYKCKVSNKALPAPIEKTISKA | | | |
| | | KGQPREPQVYTLPPSRDELTKNQVSLTCLVKGFY | | | |
| | | PSDIAVEWESNGQPENNYKTTPPVLDSDGSFFLY | | | |
| | | SKLTVDKSRWQQGNVFSCSVMHEALHNHYTQKSL | | | |
| | | SLSPGK | | | |
| | | (SEQ ID NO: 2633) | | | |

TABLE 45D
Information regarding the antibodies and Fabs used in the Examples

| Tables 45A-C | Examples 20-24 | | Table 49 |
|---|---|---|---|
| Antibody ID | FAb IDs | mAbs IDs | mAbs ID |
| 06BLM | 54BLH | 69BLH | 42BMD |
| 07BLM | 55BLH | 70BLH | 43BMD |
| 08BLM | 56BLH | 71BLH | 44BMD |
| 09BLM | 57BLH | 72BLH | 45BMD |
| 10BLM | 58BLH | 73BLH | 46BMD |
| 11BLM | 59BLH | 74BLH | 47BMD |
| 12BLM | 60BLH | 75BLH | 48BMD |
| 13BLM | 61BLH | 76BLH | 49BMD |
| 14BLM | 62BLH | 77BLH | 50BMD |
| 15BLM | 63BLH | 78BLH | 51BMD |
| 16BLM | 64BLH | 79BLH | 52BMD |
| 17BLM | 65BLH | 80BLH | 53BMD |
| 18BLM | 66BLH | 81BLH | 54BMD |
| 19BLM | 67BLH | 82BLH | 55BMD |
| 20BLM | 68BLH | 83BLH | 56BMD |

TABLE 46
P3U1 functional assay of 15 affinity matured antibodies

| | ELISA | ELISA | | |
|---|---|---|---|---|
| | assay #1* | assay *2 | Average | Standard |
| | EC50 | EC50 | EC50 | Deviation |
| Antibody | (μg/mL) | (μg/mL) | (μg/mL) | (SD) |
| 20E6 | 2.376 | 8.905 | 5.641 | 4.617 |
| parental | | | | |
| 06BLM | 0.1126 | 0.137 | 0.125 | 0.017 |
| 07BLM | 0.03324 | 0.119 | 0.076 | 0.061 |
| 08BLM | 0.05389 | 0.098 | 0.076 | 0.031 |
| 09BLM | 0.0295 | 0.130 | 0.080 | 0.071 |
| 10BLM | 0.03324 | 0.134 | 0.083 | 0.071 |
| 11BLM | 0.05389 | 0.112 | 0.083 | 0.041 |
| 12BLM | 0.0295 | 0.096 | 0.063 | 0.047 |
| 13BLM | 0.07854 | 0.105 | 0.092 | 0.018 |
| 14BLM | 0.1694 | 0.146 | 0.158 | 0.016 |
| 15BLM | 0.2191 | 0.243 | 0.231 | 0.017 |
| 16BLM | 0.08161 | 0.126 | 0.104 | 0.032 |
| 17BLM | 0.03568 | 0.089 | 0.063 | 0.038 |
| 18BLM | 0.09822 | 0.006 | 0.052 | 0.065 |
| 19BLM | 0.1525 | 0.205 | 0.179 | 0.037 |
| 20BLM | 0.05738 | 0.008 | 0.033 | 0.035 |

### Example 17. Binding of LAP/TGFβ1 Antibodies to Tumor Infiltrating Immune Cells

This example analyzed the binding to tumor Infiltrating immune cells of parental humanized 20E6 antibody and the selected affinity matured humanized antibodies 06BLM, 07BLM, 08BLM, 09BLM, 10BLM, 11BLM, 12BLM, 13BLM, 14BLM, 15BLM, 16BLM, 17BLM, 18BLM, 19BLM, and 20BLM. Fresh human kidney and lung tumors (0.5-1 g) from nine patients were sourced from commercial vendors. Each individual sample was placed in a 100 mm dish and cut into small pieces. The samples were then digested for 20 minutes at 37° C. with 10-20 mL tumor digestion medium consisting of RPMI-1640 (Gibco, Catalog no. 11875-093) supplemented with 100 U/mL of collagenase I (Worthington, Catalog no. 4196) and 400 U/mL of DNAse I (Worthington, Catalog no. 2060). The tumor digest sample was passed through 70 m mesh filter and washed with RPMI-1640 supplemented with 10% fetal bovine serum (FBS; Gibco, Catalog no. 16140-071), 1% penicillin/streptomycin (Gibco, Catalog no. 15140-122) and 1% L-glutamine (Gibco, Catalog no. 25-005-CV) to obtain a single cell suspension. The cells were pelleted by centrifuging at 1300 rpm for 7 minutes at 4° C. and then lysed with 2 mL of ammonium-chloride-potassium lysing solution (Life Technologies, Catalog no. A10492-01) for 5 minutes at room temperature. At the end of the incubation period, the lysed cells were washed with Stain Buffer (BD Biosciences, Catalog no. 554657) and counted using a trypan blue exclusion assay.

Approximately one million viable cells were aliquoted into appropriate wells of 96-well V bottom deep well block (CoStar/Corning, Catalog no. 3960). The cells were pelleted by centrifuging at 400G for 5 minutes at 4° C. The cell pellets were re-suspended in 100 μL of a blocking buffer mix consisting of stain buffer containing 10% human TruStain FcX block solution (BioLegend, Catalog no. 422302) and 500 mouse serum (Jackson Immunoresearch, Catalog no. 015-000-120). The cells were incubated in the stain buffer at 4° C. for minimum of 30 minutes. At the end of the blocking period, the cells were incubated with 100 μL of Alexa Fluor 647 fluorescent compound labeled anti-LAP/TGFβ1 antibodies or an Alexa Fluor 647 fluorescent compound labeled corresponding isotype control antibody for 30 minutes at 4° C. See Table 47.

TABLE 47
Anti-LAP/TGFβ Antibody Candidates and Isotypes

| | | | | Degree of |
|---|---|---|---|---|
| Antibody | Lot No. | Antibody Description | Fluorophore | Labeling |
| Hu IgG1 | 98BHI | Humanized × [RSV] mAb (Synagis) IgG1 | AF647 | 4.1 |
| LALA DS | | L234A L235A D265S/Kappa (CX) | | |
| Parental | 54BJS | Humanized × [LAP-TGFb1_H] mAb (20E6) | AF647 | 3.7 |
| 20E6 mAb | | IgG1 L234A L235A D265S/Kappa (PS) | | |
| 12BLM | 48BMD | Humanized × [LAP-TGFb1_H] mAb | AF647 | 4.4 |
| | | (20E6_VH_W33D_I58V/VL_T30Y R53V_ | | |
| | | H55E S56T_T93E) IgG1 L234A L235A | | |
| | | D265S/Kappa (CX) | | |
| 07BLM | 43BMD | Humanized × [LAP-TGFb1_H] mAb | AF647 | 3.5 |
| | | (20E6_VH_W33D_K59R/VL_T30Y N31S_ | | |
| | | R53N H55E_S56T) IgG1 L234A L235A | | |
| | | D265S/Kappa (CX) | | |
| 11BLM | 47BMD | Humanized × [LAP-TGFb1_H] mAb | AF647 | 3.6 |
| | | (20E6_VH_W33D_K59R/VL_T30Y R53V_ | | |
| | | H55E_S56T) IgG1 L234A L235A D265S/Kappa (CX) | | |
| 15BLM | 51BMD | Humanized × [LAP-TGFb1_H] mAb | AF647 | 4.5 |
| | | (20E6_VH_W33D/VL_T30Y_H55E_S56T) | | |
| | | IgG1 L234A L235A D265S/Kappa (CX) | | |
| 19BLM | 55BMD | Humanized × [LAP-TGFb1_H] mAb | AF647 | 3.3 |
| | | (20E6_VH_W33D_W99Y Y101H/VL_T30 | | |
| | | Y_H55E_S56T) IgG1 L234A L235A D265S/Kappa (CX) | | |
| 18BLM | 54BMD | Humanized × [LAP-TGFb1_H] mAb | AF647 | 3.5 |
| | | (20E6_VH_W33D_W99Y_Y101H/VL T30 | | |
| | | Y_H55E_S56T) IgG1 L234A L235A D265S/Kappa (CX) | | |

Samples were washed with Dulbecco's phosphate-buffered saline (DPBS, Gibco, Catalog no. 14190-144) and incubated with 100 μL of 0.5% solution of fixable viability dye (eBioscience, Catalog no. 65-0866-18) in DPBS for 20 minutes at 4° C. Samples were washed with 2 mL of stain buffer and then incubated with 50 μL of a blocking buffer mix containing stain Buffer with 10% human TruStain FcX blocking solution (BioLegend) and 4% mouse serum for 10 minutes at 4° C. Human TruStain FcX™ is specially formulated for blocking the FcR-involved unwanted staining without interfering with antibody-mediated specific staining of human cells. Samples were then incubated with 150 μL of a Flow cytometry panel containing fluorescently labeled antibodies (Table 48) to surface markers for 30 minutes at 4° C.

TABLE 48
Surface markers for Identification of
Immune Cell Subsets by Flow Cytometry

| Targeted | | | |
|---|---|---|---|
| antigen | Fluorophore | Vendor | Catalog No. |
| CD8 | BUV395 | BD Biosciences | 563795 |
| CD3 | BUV496 | BD Biosciences | 750969 |
| CD4 | BUV737 | BD Biosciences | 612748 |
| CD66b | FITC | BioLegend | 305104 |
| CD11b | PerCP/Cy5.5 | BioLegend | 101228 |
| CD127 | PE-eF610 | eBiosciences | 61-1278-42 |
| CD25 | PE/Cy7 | BD Biosciences | 335789 |
| CD45 | AF700 | BioLegend | 304024 |
| Fixable | eF506 | eBiosciences | 65-0866-18 |
| Viability Dye | | | |
| CD56 | BV650 | BD Biosciences | 564057 |
| CD19 | BV711 | BD Biosciences | 563036 |
| CD14 | BV785 | BioLegend | 301840 |

Samples were then washed with stain buffer and were fixed with 1.6% paraformaldehyde (Electron Microscopy Sciences, Catalog no. 15710) for 15 minutes at 4° C. Cells were again washed, pelleted and resuspended in 200 μL of stain buffer and detected using a Becton Dickinson Fortessa flow cytometer.

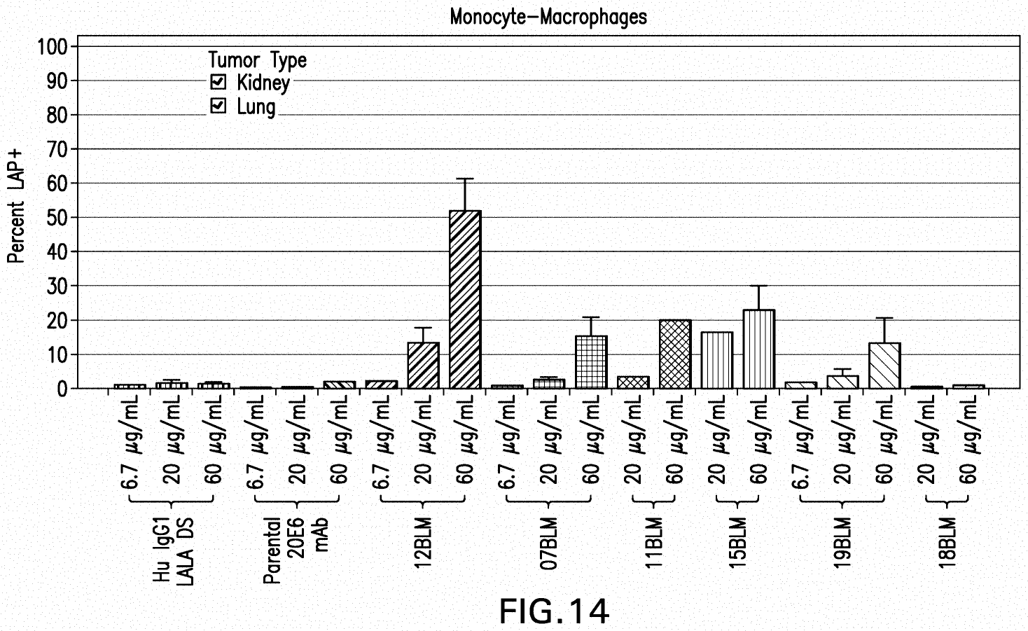

Data analysis was performed using a Flow Jo V10.6.2 software, which is a software capable of analyzing flow cytometry data. Any dead cells were excluded from analysis based on staining with an appropriate viability dye. Cell surface staining with CD45 was used to identify total immune cells. Staining with CD11b was used to separate myeloid cells from non-myeloid cells. Within the myeloid population, monocyte macrophages were identified as being CD14 positive and CD66b negative. Lymphocytes were identified from the non-myeloid population based on forward and side scatter and were divided into B cells, T cells and NK cells using CD19, CD56 and CD3 antibodies, respectively. The CD3+ T cells were then further subdivided into CD4+ and CD8+ cells. Regulatory T cells were identified as being CD4 and CD25 positive and CD127 low. Total LAP expression on monocyte macrophages were evaluated by gating on the subset positive for AF647 labeled anti-LAP/TGFβ1 antibodies and expressed as percent positive of CD14+CD66b− myeloid cells. Binding data to the tumor infiltrating immune cells for the anti-LAP/TGFβ1 parental antibody and the selected affinity matured humanized antibodies are shown in FIG. 14.

### Example 18. Binding of the Affinity Matured LAP-TGFB Antibodies to Various Populations of Human and Rhesus Whole Blood and to Primary Keratinocytes and Hepatocytes

### Materials and Methods

### Test Articles, Control Antibodies, Whole Blood, Keratinocytes, and Hepatocytes

Six anti-LAP-TGFB affinity matured 20E6 clones, parental 20E6, and anti-RSV antibodies were labeled with Alexa Fluor 647 fluorescent dye (AAT Bioquest) with a degree of labeling between 3-5. The other control antibodies Trastuzumab (anti-HER2/neu mAb), Natalizumab (anti-alpha-4 integrin mAb) were labeled with Dylight-650 following the vendor's protocol (Dylight 650 labeling kit; cat #84535). Human whole blood was obtained from the in-house volunteer donor program at Merck Research Laboratories (South San Francisco, Calif., USA). Fresh whole blood (WB) (10 ml each) was collected from 2 healthy donors in K2-EDTA vacutainer tubes on the day of the experiment. Rhesus whole blood was obtained from ValleyBio Systems. The vendor collected WB from 2 healthy rhesus donors in K2-EDTA tubes and shipped it to Merck Lab within 2 hours of blood draw. Multiple donor vials of normal adult human epidermal keratinocytes (NHEK) were obtained from PromoCell (Cat #C-12001, C-12003). Donors of adult normal human hepatocytes (NHH) were obtained from LifeNet Health (cat #MTOXH1005).

### Whole Blood Sample Preparation for Flow Cytometry

All the test (parental humanized 20E6 antibody and the selected affinity matured humanized antibodies) and control antibodies were diluted to 100 ug/ml (10× concentration) in DPBS (Dulbecco's phosphate buffered saline, #14190-144). See Table 49.

TABLE 49
Information on AF-647 labeled test antibodies
and DL650 labeled control antibodies

| Antibody | |
|---|---|
| identifier | |
| in this | |
| example | Further information |
| 43BMD | Humanized × [LAP-TGFb1_H] mAb (20E6-_VH_W33D_K59R/ |
| | VL_T30Y_N31S_R53N_H55E_S56T) IgG1L234A L235A |
| | D265S/Kappa (CX) |
| 47BMD | Humanized × [LAP-TGFb1_H] mAb (20E6-_VH W33D_K59R/ |
| | VL_T30Y_R53V_H55E_S56T) IgG1L234A |
| | L235A D265S/Kappa (CX) |
| 48BMD | Humanized × [LAP-TGFb1_H] mAb (20E6-_VH_W33D_158V/ |
| | VL_T30Y_R53V_H55E_S56T_T93E) IgG1 L234A L235A |
| | D265S/Kappa (CX) |
| 51BMD | Humanized × [LAP-TGFb1_H] mAb (20E6 |
| | VH W33D/VL_T30Y_H55E_S56T) IgG1 L234A L235A D265S/ |
| | Kappa (CX) |
| 54BMD | Humanized × [LAP-TGFb1_H] mAb (20E6-_VH_W33D_W99Y_Y101H/ |
| | VL_T30Y_H55E_S56T) IgG1L234A L235A D265S/Kappa (CX) |
| 55BMD | Humanized × [LAP-TGFb1_H] mAb (20E6-_VH_W33D_W99Y_Y101H/ |
| | VL_T30Y_R53Y_H55E_S56T) IgG1 L234A L235A D265S/Kappa (CX) |
| 54BJS | Humanized × [LAP-TGFb1_H] mAb (20E6) IgG1 L234A L235A |
| | D265S/Kappa (PS) |
| 38BLI | Human × [LAP-TGFb1_H] mAb (b1-Ab6) IgG4 S228P (CX) |
| 98BHI | Humanized × [RSV] mAb IgG1 L234A L235A D265S/Kappa (CX) |

Dylight-650 labeled controls:

### 96BAL: Humanized×[ITGA4_H] mAb IgG4/Kappa (MY)

### 33AWX: Humanized×[ERBB2_HH] mAb IgG1/Kappa (CS)

These labeled antibodies were incubated in a 96-well assay blocked with WB at a concentration of 10 μg/mL (final concentration) for 30 minutes at 4° C. To check binding to RBCs and platelets, 3 additional concentrations were tested whereas binding to WBCs was assessed only at 10 μg/mL. Wells with WB only was included for untreated controls.

### Human Red Blood Cells (RBCs) and Platelet Staining:

An aliquot from the incubated WB was used for RBCs staining and platelets staining was performed using a flow cytometry panel (Table 50; 30 min at 4° C.).

TABLE 50
Flow cytometry panel information -

| Antigen | Fluorophore | Vendor | Catalog # |
|---|---|---|---|
| CD42a | BV421 | BD Biosciences | 565444 |
| CD235a | PE-Cy7 | BD Biosciences | 563666 |
| CD45 | FITC | BD Biosciences | 555482 |
| GARP | PE | Biolegend | 352054 |

Post incubation the treated, the stained blood samples were treated with a fixed agent (1% paraformaldehyde; Alfa Aesar, PFA16% Cat #43368) for 15 minutes at room temperature. A volume (1 ml) of FACS buffer (BD biosciences; #554656) was added to the samples and the samples were analyzed using a Becton Dickinson (BD) LSRII flow cytometer within 4 hours.

### Human White Blood Cells (WBCs) Staining:

The RBCs in the assay block containing the remainder of treated WB were lysed with 2 ml each of Ammonium-Chloride-Potassium (ACK) lysing buffer (GIBCO, Cat #A10492-01) by incubating the assay block for 5-7 minutes at room temperature. The assay block was centrifuged at 1500 rpm for 5 minutes and the supernatant was aspirated. The lysing of RBCs was repeated, and the cells were washed with 2 ml of DPBS. The cell pellet was re-suspended in 200 uL of DPBS containing eFLuor506 dye diluted 1:500 (eBiosciences, Fixable viability dye; Cat #65-0866-14) and incubated for 15-20 min at 4° C. The eFluor 506 organic dye is a violet-laser excitable fluorophore that has an emission peak of 506 nm. After incubation the assay block was washed with 1 ml of FACS buffer. The cells were blocked with 50 uL FACS buffer containing Fc block solution for 10 minutes at 4° C. Investigators used 36 uL FACS buffer 10 uL human Fc block (Miltenyi Biotec, Cat #120-000-442) and 4 uL normal mouse serum (Jackson ImmunoResearch Inc. Cat #015-000-120) per reaction. The cells were incubated with the following panel of commercial antibodies (see Table 51 below) for 30 minutes at 4° C. Post incubation, the cells were washed, fixed in 1% paraformaldehyde for 30 minutes at room temperature. The cells were then washed and re-suspended in FACS buffer prior to detecting using a BD LSR Fortessa™ X20 flow cytometer.

TABLE 51
Information regarding the panel of commercial
antibodies utilized in WBC staining

| Antigen | Fluorophore | Vendor | Catalog # |
|---|---|---|---|
| CD20 | FITC | BD Biosciences | 555622 |
| CD3 | Pacific Blue | BD Biosciences | 558124 |
| CD4 | PE/CF594 | BD Biosciences | 562402 |
| CD8 | BUV395 | BD Biosciences | 563795 |
| CD56 | PE | BD Biosciences | 555516 |
| CD14 | BV785 | BioLegend | 301840 |
| CD15 | PerCpCy5.5 | BD Biosciences | 560828 |

### Rhesus RBCs, Platelet and WBCs Staining

The above protocol was followed for staining of RBCs, platelets and WBCs from healthy rhesus monkey whole blood with the following exceptions. The human CD235a RBC marker does not cross-react with rhesus blood. The rhesus RBCs are were separated/analyzed using a on forward scatter area (FSC-A) density plot vs and side scatter area (SSC-A) density plot. The A anti-CD45 antibody (BD biosciences, cat #557803) is was used for gating/separating out whether the lymphocytes is was non-human primate specific. NKG2A (CD159) was used as the lineage marker for rhesus NK cells (Miltenyi Biotec cat #130-113-566). The CD15 antibody does not cross-react to rhesus granulocytes. The rhesus granulocytes were selected using aby FSC-A density plot and a vs and SSC-A density plot.

### Hepatocyte and Keratinocyte Staining

All the test and control labeled antibodies were diluted to 20 ug/ml (2× concentration) in DPBS (Catalog No. #14190-144). Multiple frozen vials of primary keratinocytes from 2 donors were thawed and transferred to a tube containing 10 mL Dulbecco's modified Eagle's medium (DMEM, Gibco cat #11995-065) supplemented with 2% FBS. The cells were centrifuged (500 g ×1500 RPM, 4° C.) for 5 minutes and any supernatant was discarded. The cells washed and re-suspended with DPBS. The cell viability, and cell number were determined and recorded. Approximately 500 thousand cells were transferred to a 96 well V-bottom plate (USA Scientific, Inc; cat #1896-5110) for an unstained sample. Cell viability was determined by adding eFLuor™506 (1:500 dilution; Fixable Viability Dye eFluor506, eBioscience #65-0866-18) dye to rest of the remaining cells. The cells and dye were incubated at 4° C. for 30 minutes. (The cells were washed and re-suspended in FACS buffer and were divided into wells of a 96-well plate. The cells were incubated for 30 minutes at 4° C. with labeled test antibodies or control antibodies concentrated at 10 μg/mL. After incubation, the antibody administered cells were washed with FACS buffer solution and were blocked for 10 minutes at 4° C. with 50 uL FACS buffer containing Fc block. Investigators used 36 uL of FACS Buffer, 10 uL human Fc block solution (Miltenyi Biotec, Cat #120-000-442) and 4 uL normal mouse serum (Jackson ImmunoResearch Inc. Cat #015-000-120) per each reaction. The cells were incubated with a flow cytometry staining panel [EGFR (for keratinocytes) fluorophore PerCpCy5.5 Biolegend; ASGPR1 (for hepatocytes) fluorophore PE BD Biosciences)] for 30 minutes at 4° C.

The cells were washed twice with FACS buffer and were centrifuged (500 times gravity), at 4° C. for 5 minutes. The cells were treated with 1% paraformaldehyde (Alfa Aesar, cat #43368) fixing agent. The cells were pelleted and re-suspended in FACS buffer before being analyzed using an BD LSRII or Fortessa flow cytometer.

The protocol utilized for checking binding to hepatocytes was the same as described above with the following differences; upon being thawed, the cells were transferred to 40 mL of hepatocyte thawing media (Invitrogen; cat #CM7000) instead of the DMEM described in the protocol above. In addition, the specific lineage marker utilized for staining human hepatocytes was an anti-ASGPR1 antibody.

### FACS Data Analysis

Data analysis was performed using a FlowJo V10 software system. Dead cells were excluded from analysis and viable cells identified using a viability dye stain. Peripheral blood mononuclear cells were identified utilizing a forward and side scatter (FSC vs SSC) gating analysis. Lymphocytes were separated into B cells and T cells by CD20lineage markers and CD3 lineage markers, respectively. The non-B cell T population was further separated into the following subgroups: CD56+NK (for human), NKG2A (for rhesus) and CD14+ monocytes. CD3+ T cells were then further subdivided into CD4+ and CD8+ cells. The granulocytes were gated/separated into a CD15+ population in human WB samples and FSC-A and SSC-A gating analysis for rhesus samples.

Dead hepatocytes or keratinocytes were excluded by staining cell samples with a viability dye, and gated using FSC-A and SSC-A gating analysis after singlet selection. Singlets were gated on their specific lineage markers.

All the cell subset populations were further gated on AF647+ cells or Dylight 650+ cells. The mean fluorescent intensity (MFI) and percent positive for AF647+ or Dylight+ cells were determined.

### Results:

### Human RBCs and Platelets:

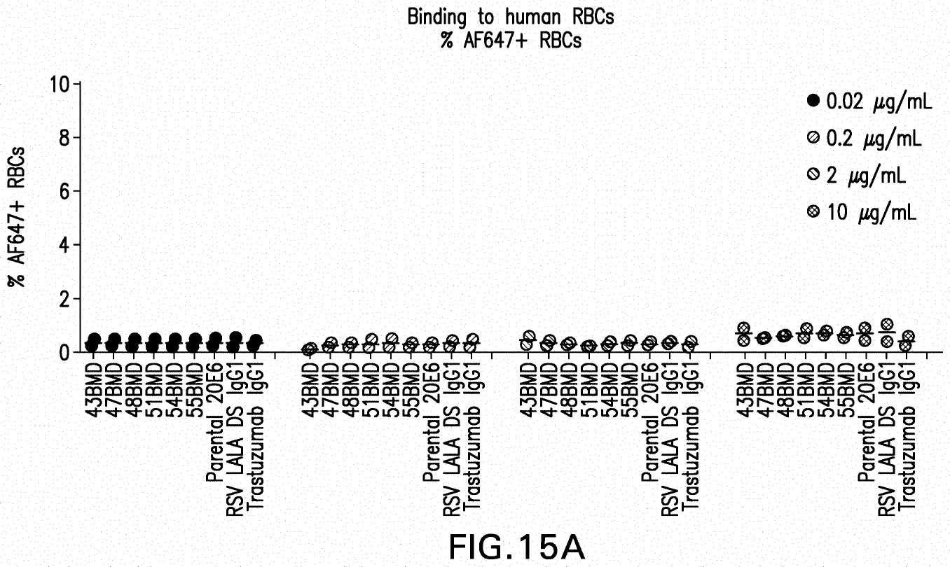

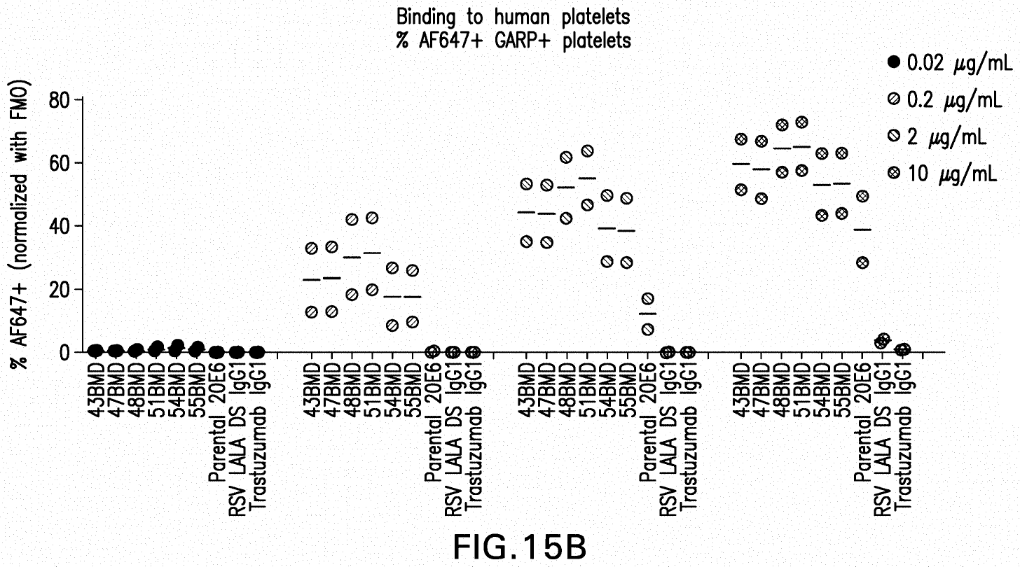

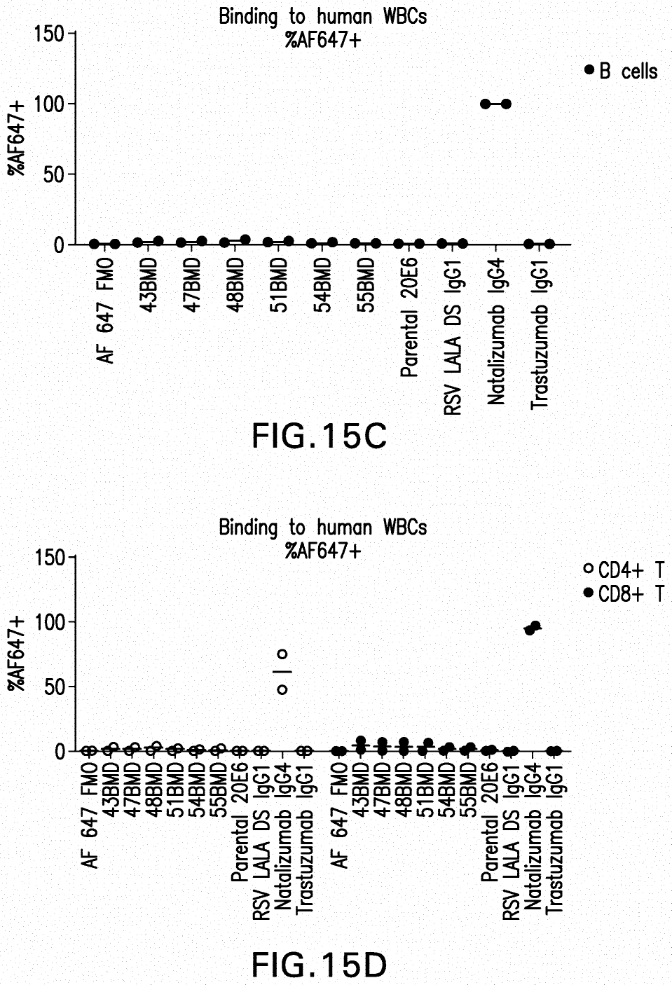

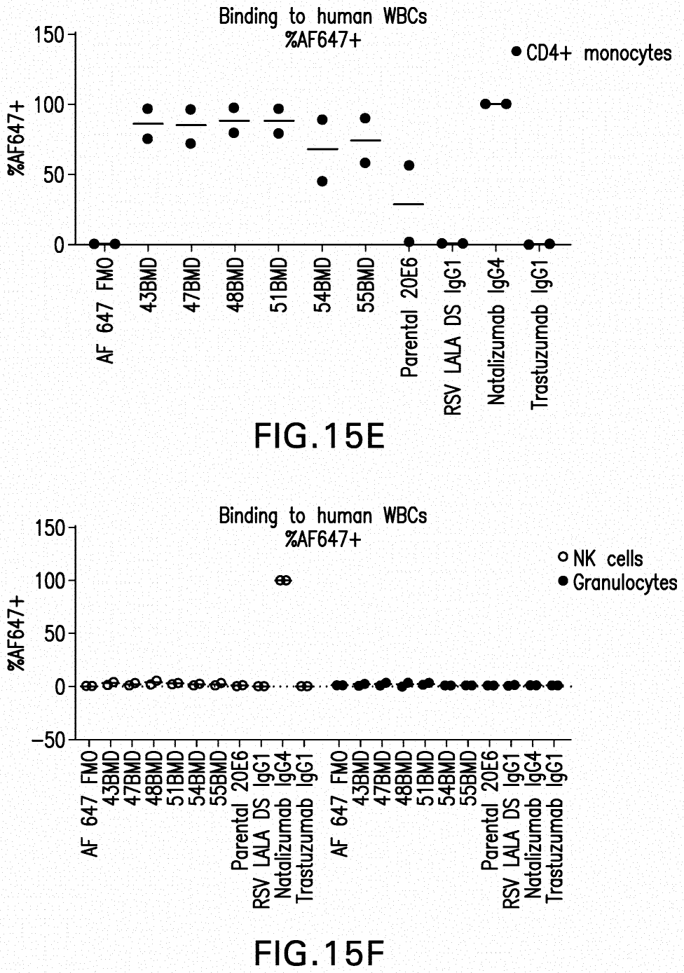

Data show that each of the affinity matured LAP-TGFβ antibodies, parental 20E6 antibody, anti-RSV antibody, anti-ER2/neu antibody Trastuzumab did not bind human RBCs (FIG. 15A). This lack of binding was observed across each of the concentrations tested: 0.02, 0.2, 2, and 10 ug/mL. All the 6 affinity matured LAP-TGFβ analyzed in this Example bound to human platelets in a dose dependent manner at 0.2, 2, and 10 ug/mL(FIG. 15B). The percent positive AF647 platelet binding for all the 6 antibodies was greater than the binding seen with parental 20E6 antibody for each concentration tested in this Example.

### Human WBCs:

The data show that the affinity matured LAP-TGFβ antibodies, parental 20E6, trastuzumab (each at a concentration of 10 μg/mL) did not bind to human CD20+B, CD4+T, CD8+T, CD56+ NK cells and granulocytes. See FIG. 15C, FIG. 15D, and FIG. 15F. Data show that the LAP-TGFB antibodies target CD14+ monocytes (FIG. 15E). This binding of CD14+ monocytes by the selected affinity matured anti-LAP-TGFB humanized antibodies was observed to be greater than what was observed with the parental 20E6 humanized antibody. Natalizumab at a concentration of at 10 μg/mL was utilized as a positive control in many of the binding experiments, and data show that this antibody bound to all of the tested WBCs populations.

### Rhesus RBCs and Platelets:

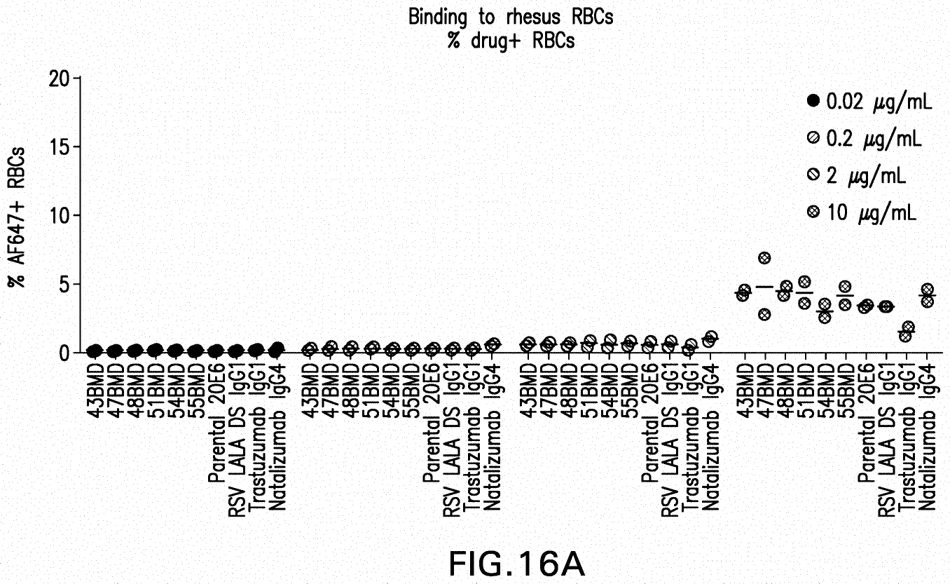

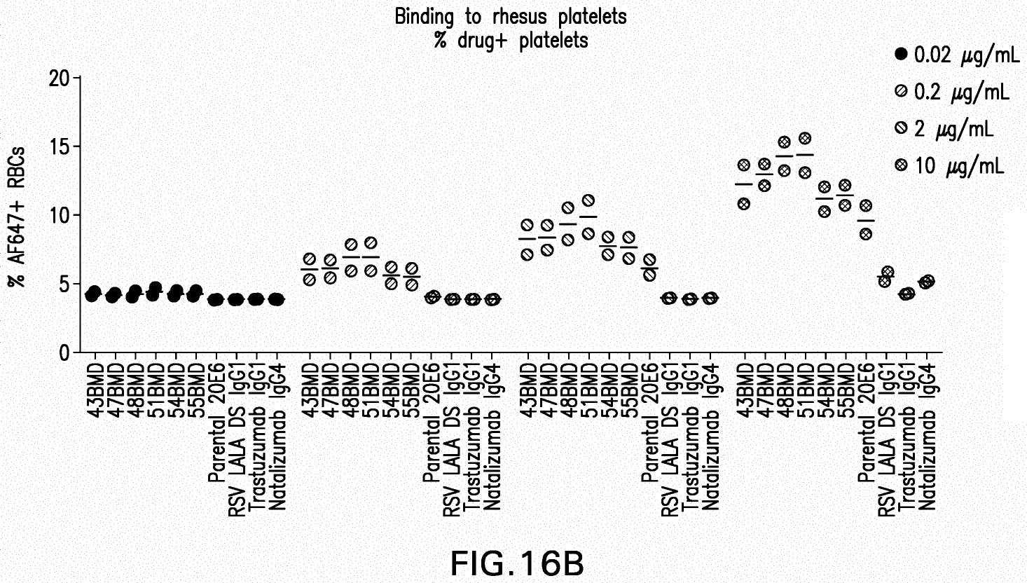

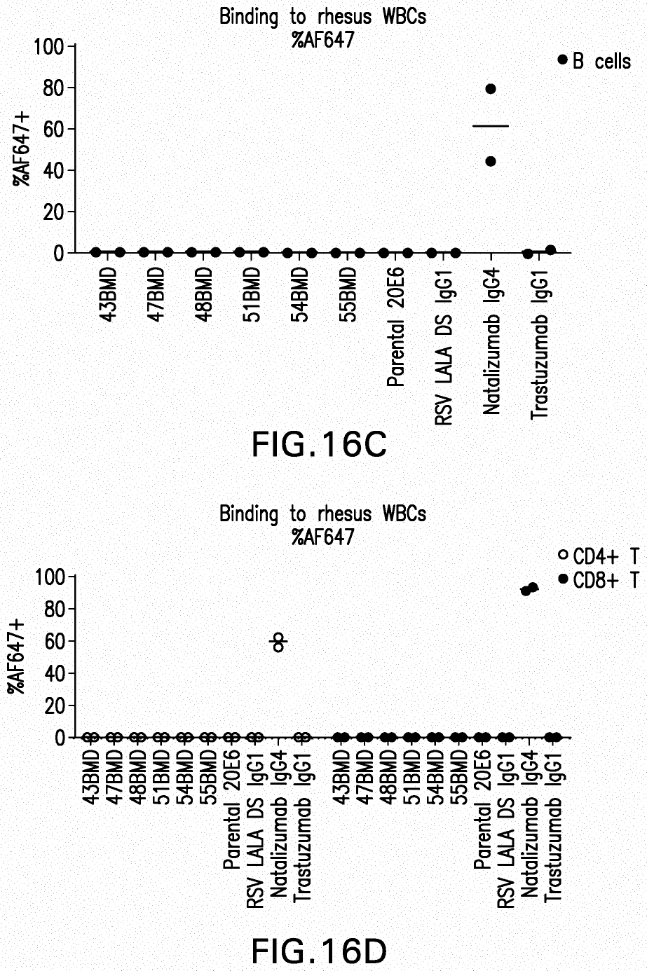

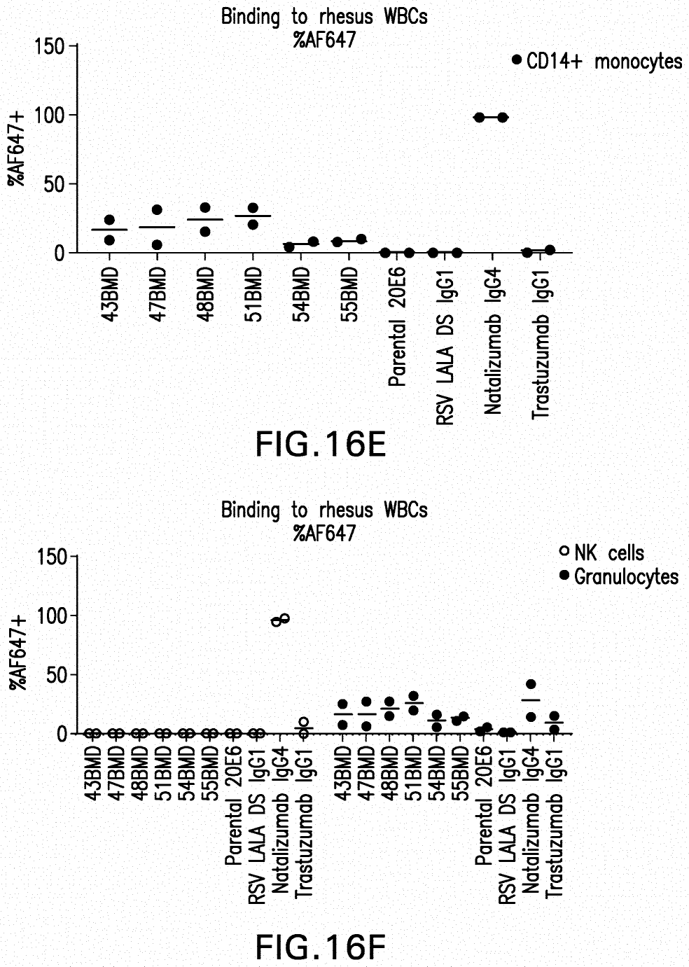

Data show that (at the 4 concentrations tested) each of the antibodies (i.e., the selected six affinity matured anti-LAP-TGFB antibodies, parental 20E6 humanized antibody, anti-RSV antibody, and HER2/neu antibody Trastuzumab) did not bind rhesus RBCs (FIG. 16A). All the affinity matured anti-LAP-TGFB antibodies were observed to bind to rhesus platelets in a dose dependent manner at 0.2, 2, and 10 ug/mL (FIG. 16B). The percent positive AF647 platelet binding for each of the anti-LAP-TGFB antibodies was observed to be greater than the positive AF647 platelet binding observed for the parental 20E6 humanized antibody.

### Rhesus WBCs:

The affinity matured anti-LAP-TGFB antibodies, parental 20E6 antibody, and Trastuzumab (each at a concentration of 10 μg/mL) were observed not to have bound to rhesus CD20+B, CD4+T, CD8+T, NKG2A+ NK cells (FIG. 16C, FIG. 16D, and FIG. 16F). Data show that the affinity matured anti-LAP-TGFβ antibodies do show a range of on-target binding to CD14+ monocytes (FIG. 16E) and binding to granulocytes(FIG. 16F). The observed binding for the anti-LAP-TGFB antibodies was greater than what was observed for cells treated with the parental 20E6 antibody. Natalizumab was utilized as a positive control and data show that this antibody bound to all the tested WBCs populations.

### Human Hepatocytes and Keratinocytes:

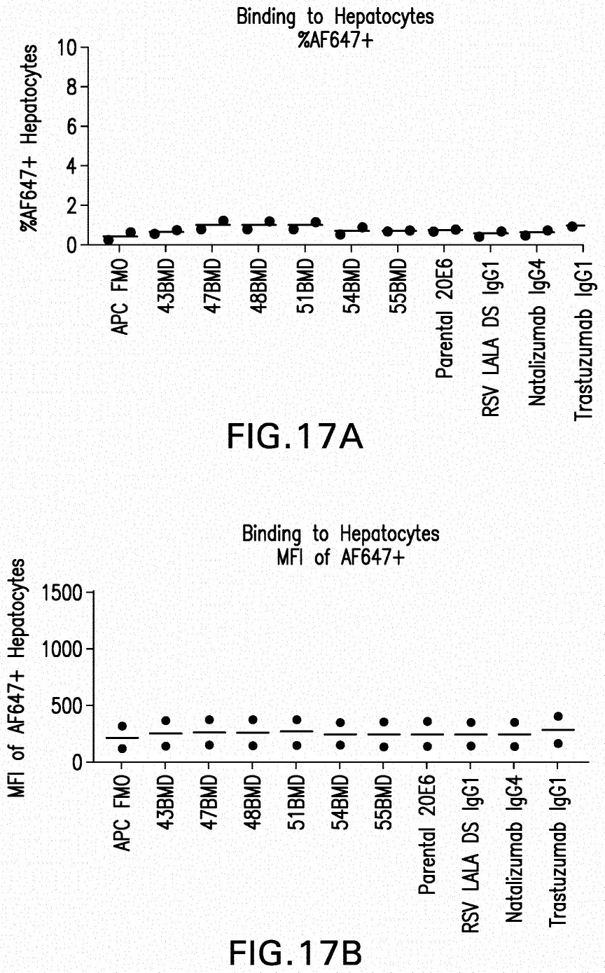

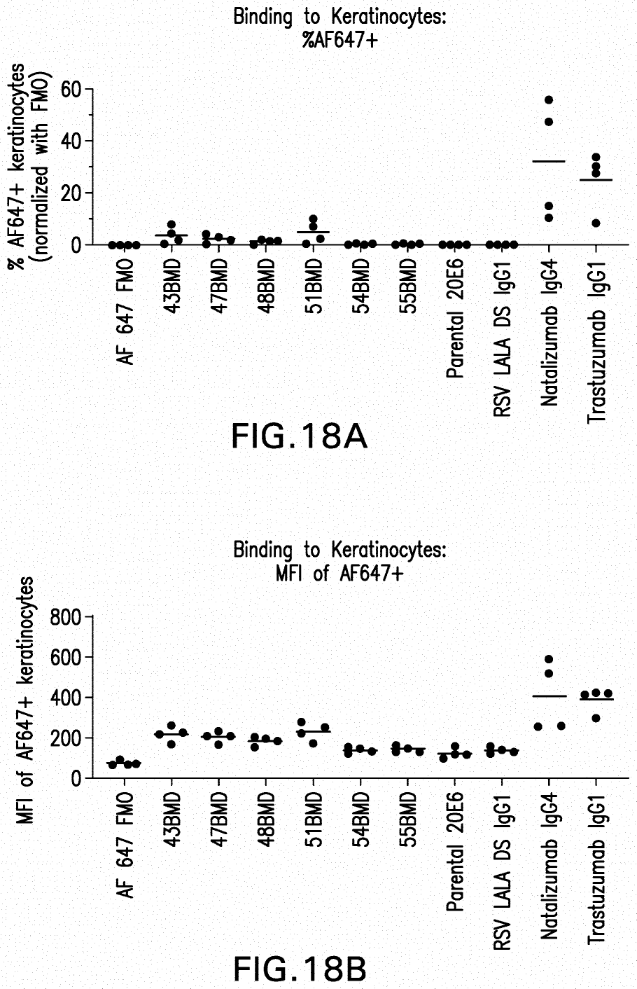

It was observed that at the tested 10 ug/mL concentration each of the six affinity matured anti-LAP-TGFβ antibodies, parental 20E6 antibody, anti-RSV antibody, trastuzumab, and natalizumab did not bind human hepatocytes (FIG. 17A and FIG. 17B). At the tested 10 ug/mL concentration all the affinity matured anti-LAP-TGFB antibodies, parental 20E6, and anti-RSV antibodies do not bind human keratinocytes (FIG. 18A and FIG. 18B). Natalizumab and trastuzumab are positive control antibody.

### Example 19. Assessment of the Human Platelet Activation with the Affinity Matured LAP-TGFB Antibodies

Six anti-LAP TGFβ affinity matured 20E6 clones and parental 20E6 antibody were tested for their ability to activate platelets.

### Materials and Methods

The following lists additional controls used in the assays in this Example: Anti-human CD9 Tetraspanin, MRP-1, DRAP-24 (Biolegend); TRAP-6 trifluoroacetate salt Thrombin Receptor Activator Peptide 6 (BACHEM); and Adenosine diphosphate (ADP; Chrono-Log Corporation)

### Preparation of Agonist Controls Thrombin Receptor Activator Peptide-6 and Adenosine Diphosphate

A 5 mg vial of TRAP-6 peptide was dissolved in 1 mL water to give a stock concentration of 6.676 mM. Aliquots of 20 μL were transferred to tubes and frozen at −20° C. A 2.5 mg vial of ADP was reconstituted with 5 mL of DPBS (Hyclone #SH30028.02) to give a stock concentration of 1 mM. Aliquots of 20 μL were transferred to tubes and frozen at −20° C. Working stocks of TRAP-6 and ADP were prepared by diluting the solutions from 1 mM to 200 μM in HEPES-buffered Tyrode's solution (Boston bioproducts; cat #PY-921) supplemented with 2 mM calcium chloride and 2 mM magnesium chloride. The final concentrations used in the assay were 10 μM and 20 μM for both TRAP-6 and ADP.

Extraction of Platelet Rich Plasma from Whole Blood

Human whole blood was obtained from the in-house volunteer donor program at Merck Research Laboratories (South San Francisco, Calif., USA). Since many drugs can interfere with platelet studies, the potential donors were restricted to those who have not taken aspirin for 2 weeks or any other nonsteroidal anti-inflammatory drugs for at least 48 hours before donation. The first draw of 2 mL of collected blood was discarded. The next 30 mL of blood drawn was collected into tubes with acid citrate dextrose (ACD) solution A (ACD soln A, BD Biosciences cat #364606), gently inverted 10 times, and kept at room temperature.

The ACD tubes with whole blood underwent centrifugation in the Sorvall centrifuge at 200×g for 15 minutes without brake. After the spin, the top platelet rich plasma layer (PRP) was transferred to a fresh tube.

### Platelet Activation Assay

Calcium chloride (Boston bioproducts; cat #MT-140) and magnesium chloride (VWR Lifescience; cat #E5225-100 mL) were each added to the PRP at 2 mM concentration, mixed, and incubated for 5 minutes at room temperature. All the test and the control articles were diluted to 100 μg/mL (10×) in HEPES-buffered Tyrode's solution supplemented with 2 mM calcium chloride and 2 mM magnesium chloride. The anti-LAP TGFb antibodies, trastuzumab and anti-RSV antibodies were diluted 1:5 to 20 ug/mL. Anti-human CD9, anti-human CD151, and the agonists ADP and TRAP-6 were tested at 2 concentrations.

The agonist controls, control mAb, and anti-LAP TGFb antibodies were added to appropriate wells of a 96-well assay block. PRP was added and mixed by pipetting and the mixture was incubated at room temperature for 20 minutes. An aliquot from this incubated mixture was used for platelet staining with the following flow cytometry panel (antigen CD42a fluorophore PerCp (BD Biosciences); antigen PAC-1 fluorophore FITC (BD Biosciences); antigen CD62P fluorophore PE (BD Biosciences)] for 30 minutes at 4° C. then fixed with 100 μL of 1% paraformaldehyde (Alfa Aesar; Cat #43368) for 15 minutes at room temperature. Samples were analyzed using a BD LSR II flow cytometer or BD LSRFortessa™ X20 flow cytometer.

Activation was compared to PRP that was treated the same but not incubated with antibody or agonists (no mAb control). Platelets were identified by gating on CD42a+ population. Activated platelets were identified by an increase in mean fluorescence intensity (MFI) of CD62P and PAC-1 when compared to PRP not incubated with mAb or agonist. Data was analyzed using FlowJo software, Version 10 and plotted using GraphPad Prism Software.

### Results

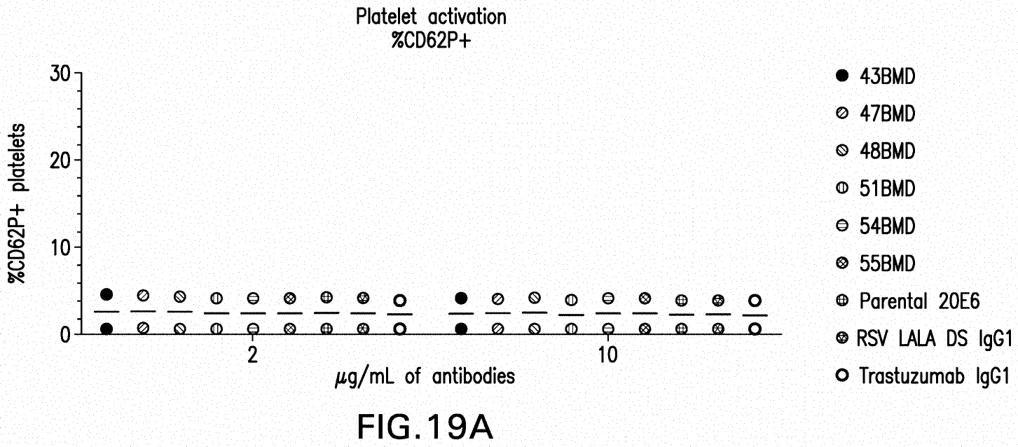

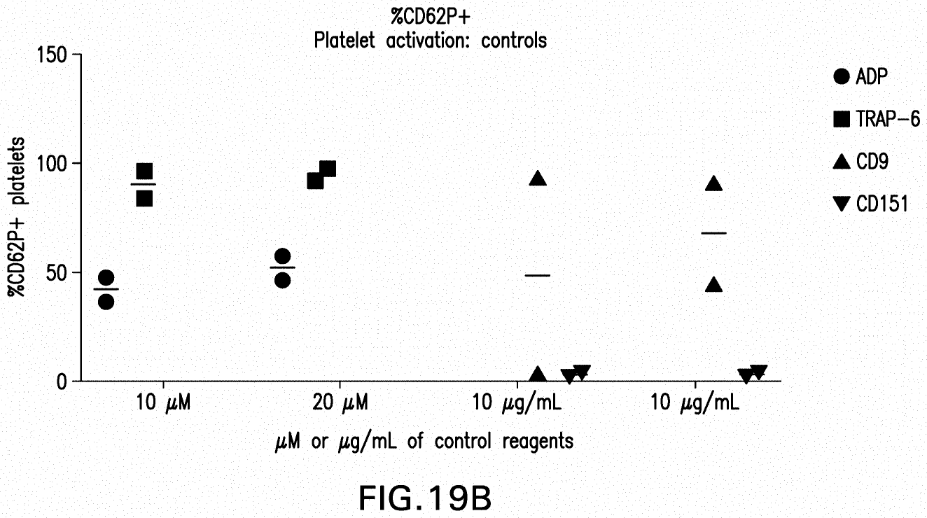

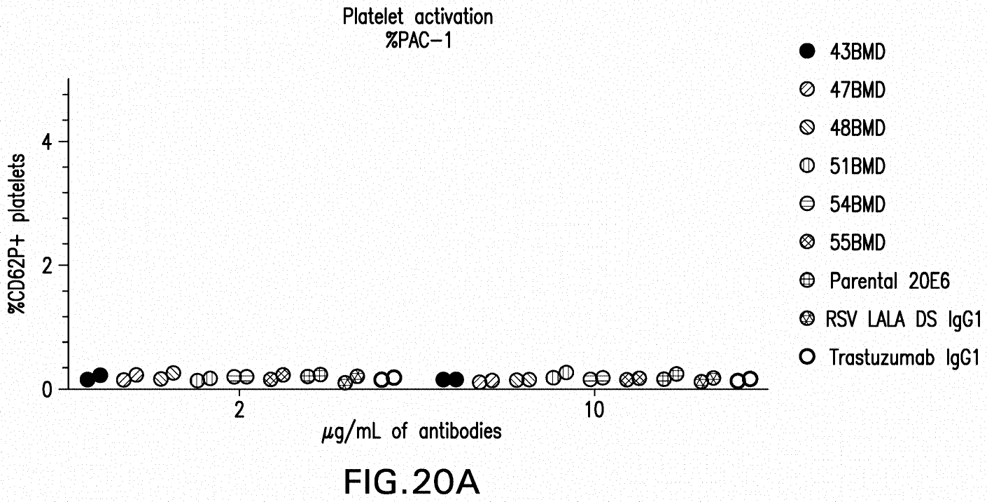

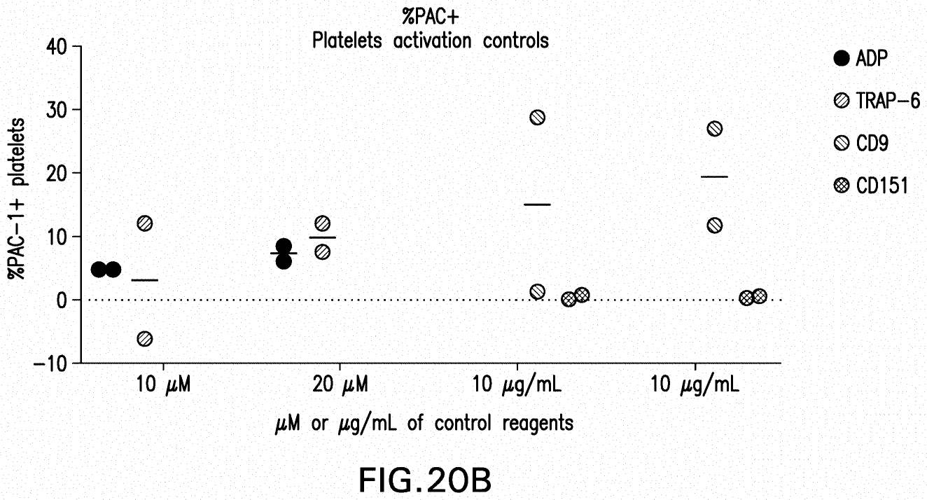

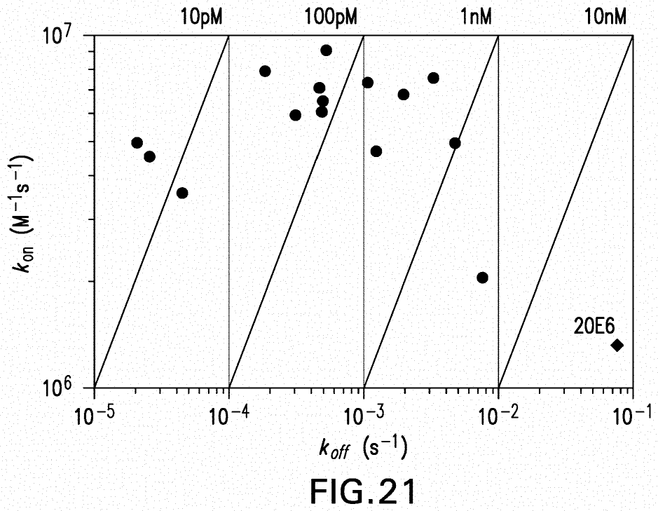

At the concentrations and condition tested, the selected affinity matured anti-LAP-TGFB antibodies, parental 20E6 antibody, and controls (i.e., anti-RSV antibody, trastuzumab and natalizumab) showed no evidence of platelet activation in PRP from multiple human donors. There was no increase in the percent of positive CD62P (FIG. 19A) or PAC-1 PRP (FIG. 20A) upon incubation with test articles, trastuzumab, or anti-RSV antibody.

Incubation of PRP with control agonists (ADP or TRAP-6) and positive control mAb (anti-human CD9 or anti-human CD151) led to an increase the percent of CD62P positive cells (FIG. 19B). Incubation of PRP with the positive control mAbs led to an increase in the percent of PAC-1 positive cells (FIG. 20A).

### Example 20. Yeast Display Mutants Binding Human LAP-TGFB Isoforms 1, 2, and 3

This Example analyzed the isoform specificity of the yeast display mutants to bind to human LAP-TGFβ isoforms 1, 2, and 3 using surface plasmon resonance.

A Series S CM4 sensor chip (Cytiva, catalog BR100534) was immobilized with an anti-human Fc capture antibody following the steps described in the kit protocol (Cytiva, catalog BR100839) on a Biacore T200 instrument with 1×HBS-EP+(Teknova, catalog H8022). Kinetic binding interactions between human LAP-TGFβ isoform 1 and yeast display mutants were performed in 1× HBS-EP+ with 0.1 mg/mL BSA pH 7.4 (Jackson Immunoresearch, catalog 001-000-162) at 25° C. Approximately 20-40 RU of human LAP-TGFβ-Fc isoform 1 was captured to the anti-human Fc surface followed by injection of 1:4 serially diluted Fab from 1000 nM to 3.91 nM (parental humanized 20E6 antibody) and 1:3 serially diluted Fab from 60 nM to 0.74 nM (mutants 59BLH, 62BLH, 65BLH-68BLH), 6 nM to 0.07 nM (mutants 55BLH, 57BLH, 58BLH, 63BLH, 64BLH), and 2 nM to 0.02 nM (mutants 54BLH, 56BLH, 60BLH, 61BLH) including 0 nM Fab. Binding specificity between human LAP-TGFβ isoforms 2 and 3 and yeast display mutants were performed in 1×HBS-EP+ with 0.1 mg/mL BSA pH 7.4 at 25° C. Approximately 20-40 RU of human LAP-TGFβ2 and 3 were captured to the anti-human Fc surface followed by injection of 1000 nM Fab including a 0 nM Fab. The binding data were double referenced by subtraction of signal from a reference (capture surface only) flow cell and the 0 nM Fab injection. Binding rate constants were determined by fitting the data with a 1:1 binding model (Cytiva Biacore T200 Evaluation software 3.0).

As shown in Table 52, the parental humanized 20E6 antibody bound to human LAP-TGFβ1 with nanomolar affinity, but no appreciable binding was observed to human LAP-TGFβ isoform 2 or 3 to 1000 nM Fab. The yeast display mutants demonstrated 16 to >10000-fold improved binding affinity to human LAP-TGFβ1 over the binding data for the parental humanized 20E6 antibody. Like the data for the parental humanized 20E6 antibody, the yeast display mutants did not bind human LAP-TGFβ isoform 2 and 3.

TABLE 52
Binding parameters for 20E6 and yeast display mutants binding to human LAP-TGFβ isoforms 1, 2, and 3

| | | LAP-TGFβ1 | | | | | |
|---|---|---|---|---|---|---|---|
| | | | | | Fold | LAP-TGFβ2 | LAP-TGFβ3 |
| | Sorting | kₒₙ | k_{off} | K_{D} | improvement | K_{D} | K_{D} |
| ID | population | (M⁻¹s⁻¹) | (s⁻¹) | (M) | in K_{D} | (M) | (M) |
| 20E6 (80BGJ) | parent | 1.32 × 10⁶ | 7.69 × 10⁻² | 5.85 × 10⁻⁸ | 1 | NB | NB |
| 54BLH | 12 hours sort | 4.96 × 10⁶ | 2.07 × 10⁻⁵ | 4.00 × 10⁻¹² | 14638 | NB | NB |
| 55BLH | 12 hours sort | 9.08 × 10⁶ | 5.28 × 10⁻⁴ | 5.90 × 10⁻¹¹ | 992 | NB | NB |
| 56BLH | 12 hours sort | 4.53 × 10⁶ | 2.57 × 10⁻⁵ | 6.00 × 10⁻¹² | 9758 | NB | NB |
| 57BLH | 12 hours sort | 6.06 × 10⁶ | 4.90 × 10⁻⁴ | 8.10 × 10⁻¹¹ | 723 | NB | NB |
| 58BLH | 2 hour P1 | 5.93 × 10⁶ | 3.13 × 10⁻⁴ | 5.30 × 10⁻¹¹ | 1105 | NB | NB |
| 59BLH | 2 hour P1 | 7.10 × 10⁶ | 4.70 × 10⁻⁴ | 6.60 × 10⁻¹¹ | 887 | NB | NB |
| 60BLH | 2 hr P1 | 7.91 × 10⁶ | 1.86 × 10⁻⁴ | 2.40 × 10⁻¹¹ | 2440 | NB | NB |
| 61BLH | 2 hour P2 | 3.57 × 10⁶ | 4.52 × 10⁻⁵ | 1.30 × 10⁻¹¹ | 4504 | NB | NB |
| 62BLH | 2 hour P2 | 4.69 × 10⁶ | 1.24 × 10⁻³ | 2.46 × 10⁻¹⁰ | 222 | NB | NB |
| 63BLH | 2 hour P2 | 6.79 × 10⁶ | 1.98 × 10⁻³ | 2.97 × 10⁻¹⁰ | 197 | NB | NB |
| 64BLH | 2 hour P2 | 6.51 × 10⁶ | 5.00 × 10⁻⁴ | 7.70 × 10⁻¹¹ | 760 | NB | NB |
| 65BLH | 2 hour P2 | 7.33 × 10⁶ | 1.07 × 10⁻³ | 1.46 × 10⁻¹⁰ | 401 | NB | NB |
| 66BLH | 2 hour P3 | 2.05 × 10⁶ | 7.64 × 10⁻³ | 3.73 × 10⁻⁹ | 16 | NB | NB |
| 67BLH | 2 hour P3 | 4.95 × 10⁶ | 4.76 × 10⁻³ | 9.61 × 10⁻¹⁰ | 61 | NB | NB |
| 68BLH | 2 hour P3 | 7.57 × 10⁶ | 3.31 × 10⁻³ | 4.38 × 10⁻¹⁰ | 134 | NB | NB |

NB—no binding observed

### Example 21. Yeast Display Mutants Binding Human LAP-TGFB Isoforms 1 at pH6.0

This Example analyzed the isoform specificity of the yeast display mutants to bind to human LAP-TGFβ1 isoform at pH6.0 using surface plasmon resonance.

A Series S CM4 sensor chip (Cytiva, catalog BR100534) was immobilized with an anti-human Fc capture antibody following the kit protocol (Cytiva, catalog BR100839) on a Biacore T200 instrument with 1× HBS-EP+ (Teknova, catalog H8022). Kinetic binding interactions between human LAP-TGFβ isoform 1 and yeast display mutants were performed in 1×HBS-EP+ with 0.1 mg/mL BSA pH 6.0 (Jackson Immunoresearch, catalog 001-000-162) at 25° C. Approximately 20-40 RU of human LAP-TGFβ-Fc isoform 1 was captured to the anti-human Fc surface followed by injection of 1:4 serially diluted Fab from 1000 nM to 3.91 nM (parental humanized 20E6 antibody) and 1:3 serially diluted Fab from 60 nM to 0.74 nM (mutants 59BLH, 62BLH, 65BLH-68BLH), 6 nM to 0.07 nM (mutants 55BLH, 57BLH, 58BLH, 63BLH, 64BLH), and 2 nM to 0.02 nM (mutants 54BLH, 56BLH, 60BLH, 61BLH) including 0 nM Fab. The binding data were double referenced by subtraction of signal from a reference (capture surface only) flow cell and the 0 nM Fab injection. Binding rate constants were determined by fitting the data with a 1:1 binding model (Cytiva Biacore T200 Evaluation software 3.0).

As shown in Table 53, Fab binding affinity at pH6.0 was slightly weaker than what was observed at pH7.4 and maybe due to slightly faster k_{off} rates. In contrast, Fabs 59BLH, 64BLH, and 65BLH were observed to have a Fab binding affinity that was slightly higher (i.e., higher binding) at pH6.0 than at pH7.4.

TABLE 53
Binding parameters for 20E6 and yeast display mutants binding to human LAP-TGFβ isoforms 1 at pH 6.0 and pH 7.4

| | | | | | | | | Fold change |
|---|---|---|---|---|---|---|---|---|
| | | pH 6.0 | | | pH 7.4 | | | in K_{D} from |
| | Sorting | kₒₙ | k_{off} | K_{D} | kₒₙ | k_{off} | K_{D} | pH 7.4 to |
| ID | population | (M⁻¹s⁻¹) | (s⁻¹) | (M) | (M⁻¹s⁻¹) | (s⁻¹) | (M) | pH 6 |
| 20E6 (80BGJ) | parent | 9.34 × 10⁵ | 9.60 × 10⁻² | 1.03 × 10⁻⁷ | 1.32 × 10⁶ | 7.69 × 10⁻² | 5.85 × 10⁻⁸ | 1.8 |
| 54BLH | 12 hours sort | 7.56 × 10⁶ | 4.73 × 10⁻⁵ | 6.00 × 10⁻¹² | 4.96 × 10⁶ | 2.07 × 10⁻⁵ | 4.00 × 10⁻¹² | 1.5 |
| 55BLH | 12 hours sort | 7.17 × 10⁶ | 7.57 × 10⁻⁴ | 1.05 × 10⁻¹⁰ | 9.08 × 10⁶ | 5.28 × 10⁻⁴ | 5.90 × 10⁻¹¹ | 1.8 |
| 56BLH | 12 hours sort | 5.55 × 10⁶ | 5.56 × 10⁻⁵ | 1.00 × 10⁻¹¹ | 4.53 × 10⁶ | 2.57 × 10⁻⁵ | 6.00 × 10⁻¹² | 1.7 |
| 57BLH | 12 hours sort | 1.54 × 10⁷ | 1.59 × 10⁻³ | 1.06 × 10⁻¹⁰ | 6.06 × 10⁶ | 4.90 × 10⁻⁴ | 8.10 × 10⁻¹¹ | 1.3 |
| 58BLH | 2 hr P1 | 6.88 × 10⁶ | 5.91 × 10⁻⁴ | 8.60 × 10⁻¹¹ | 5.93 × 10⁶ | 3.13 × 10⁻⁴ | 5.30 × 10⁻¹¹ | 1.6 |
| 59BLH | 2 hr P1 | 1.11 × 10⁷ | 5.43 × 10⁻⁴ | 4.90 × 10⁻¹¹ | 7.10 × 10⁶ | 4.70 × 10⁻⁴ | 6.60 × 10⁻¹¹ | 0.7 |
| 60BLH | 2 hr P1 | 7.23 × 10⁶ | 4.81 × 10⁻⁴ | 6.70 × 10⁻¹¹ | 7.91 × 10⁶ | 1.86 × 10⁻⁴ | 2.40 × 10⁻¹¹ | 2.8 |
| 61BLH | 2 hour P2 | 3.91 × 10⁶ | 1.21 × 10⁻⁴ | 3.10 × 10⁻¹¹ | 3.57 × 10⁶ | 4.52 × 10⁻⁵ | 1.30 × 10⁻¹¹ | 2.4 |
| 62BLH | 2 hour P2 | 5.80 × 10⁶ | 1.68 × 10⁻³ | 2.90 × 10⁻¹⁰ | 4.69 × 10⁶ | 1.24 × 10⁻³ | 2.46 × 10⁻¹⁰ | 1.2 |
| 63BLH | 2 hour P2 | 7.51 × 10⁶ | 2.77 × 10⁻³ | 3.69 × 10⁻¹⁰ | 6.79 × 10⁶ | 1.98 × 10⁻³ | 2.97 × 10⁻¹⁰ | 1.2 |
| 64BLH | 2 hour P2 | 3.49 × 10⁷ | 8.43 × 10⁻⁴ | 2.40 × 10⁻¹¹ | 6.51 × 10⁶ | 5.00 × 10⁻⁴ | 7.70 × 10⁻¹¹ | 0.3 |
| 65BLH | 2 hour P2 | 4.35 × 10⁶ | 2.62 × 10⁻³ | 6.04 × 10⁻¹¹ | 7.33 × 10⁶ | 1.07 × 10⁻³ | 1.46 × 10⁻¹⁰ | 0.4 |
| 66BLH | 2 hour P3 | 2.09 × 10⁶ | 2.21 × 10⁻² | 1.05 × 10⁻⁸ | 2.05 × 10⁶ | 7.64 × 10⁻³ | 3.73 × 10⁻¹⁰ | 2.8 |
| 67BLH | 2 hour P3 | 7.63 × 10⁶ | 1.22 × 10⁻² | 1.59 × 10⁻⁹ | 4.95 × 10⁶ | 4.76 × 10⁻³ | 9.61 × 10⁻¹⁰ | 1.7 |
| 68BLH | 2 hour P3 | 8.94 × 10⁶ | 4.54 × 10⁻³ | 5.08 × 10⁻¹⁰ | 7.57 × 10⁶ | 3.31 × 10⁻³ | 4.38 × 10⁻¹⁰ | 1.2 |

### Example 22: Kinetic Exclusion Assay Association Rate and Affinity Assessment Analysis of Yeast Display Mutants Binding Human LAP-TGFβ Isoform 1

An orthogonal kinetic exclusion assay (KinExA) method was used in this study to measure the affinities of affinity-matured anti-LAP-TGFβ1 antibodies (referred to as the constant binding partner, CBP) and human LAP-TGFβ1 (referred to as the titrant). To determine the free CBP concentration in solution, biotinylated human LAP-TGFβ1 coated PMMA (Sapidyne, catalog 440176) beads were prepared by first coating with biotin-BSA (ThermoScientific, catalog 29130) followed by streptavidin (Invitrogen, catalog S888), and a final coat with biotinylated human LAP-TGFβ1-Fc.

For association rate analysis, an experimentally optimized constant concentration of CBP and titrant, were mixed 1:1 and free CBP was measured at timed intervals ranging from 15 to 10000 seconds. The KinExA detection method was performed as follows: PMMA beads were loaded into the flow cell, a single injection of titrant human LAP-TGFβ1 and antibody CBP was flowed over the flow cell, free CBP bound by the PMMA beads was detected with goat anti-human F(ab′)₂ F(ab′)₂ fragment specific-647 conjugate (Jackson Immunoresearch, catalog 109-605-097). The data were analyzed with KinExA Pro software (version 4.4.26) where the free CBP binding signals were converted into percent free response, plotted against time (seconds) and processed with the n-curve software “Kinetics, Direct” analysis method.

For equilibrium affinity analysis, two experimentally optimized CBP concentrations were combined with 1:2 serially diluted titrant, human LAP-TGFβ1, and incubated at room temperature to achieve equilibrium. The KinExA detection method was performed as follows: PMMA beads were loaded into the flow cell, a single concentration of titrant human LAP-TGFβ1 at equilibrium with a given antibody CBP was flowed over the flow cell, free CBP bound by the PMMA beads was detected with goat anti-human F(ab′)₂ F(ab′)₂ fragment specific-647 conjugate (Jackson Immunoresearch, catalog 109-605-097). The data was analyzed with KinExA Pro software (version 4.4.26) where the free CBP binding signals were converted into percent free response and plotted against the titrant concentration series. The equilibrium K_{D} was determined by processing with n-curve software “Equilibrium” analysis method.

As shown in Table 54, the association rate and equilibrium affinity analysis by KinExA shows that anti-human LAP-TGFβ1 antibodies bind to human LAP-TGFβ1 with a range of K_{D} from 0.37 fM to 68 pM.

TABLE 54
Association rate and equilibrium parameters of affinity
mature antibodies binding to human LAP-TGFβ isoforms 1

| | | | | | | 95% | |
|---|---|---|---|---|---|---|---|
| | Sorting | kₒₙ | k_{off} | K_{D} | Error | Confidence | |
| ID | population | (M⁻¹s⁻¹) | (s⁻¹) | (M) | (%) | Interval | |
| 70BLH | 12 hours sort | 1.28 × 10⁷ | 2.82 × 10⁻⁵ | 2.24 × 10⁻¹² | 1.98 | 1.61-2.99 | pM |
| 74BLH | 2 hr P1 | 1.63 × 10⁷ | 8.14 × 10⁻⁶ | 1.33 × 10⁻¹² | 1.42 | 0.98-1.73 | pM |
| 75BLH | 2 hr P1 | 2.04 × 10⁷ | 7.71 × 10⁻⁶ | 3.67 × 10⁻¹³ | 3.31 | 0.02-0.92 | pM |
| 78BLH | 2 hour P2 | 1.09 × 10⁷ | 2.56 × 10⁻⁵ | 2.35 × 10⁻¹² | 2.67 | 0.90-4.36 | pM |
| 79BLH | 2 hour P2 | 5.03 × 10⁶ | 1.38 × 10⁻⁵ | 2.75 × 10⁻¹² | 2.95 | 1.74-4.04 | pM |
| 81BLH | 2 hour P3 | 9.83 × 10⁴ | 6.09 × 10⁻⁶ | 6.21 × 10⁻¹¹ | 2.48 | 45.40-83.20 | pM |
| 82BLH | 2 hour P3 | 2.92 × 10⁴ | 1.98 × 10⁻⁶ | 6.80 × 10⁻¹¹ | 2.65 | 46.80-95.10 | pM |

### Example 23. Direct Affinity Comparison of Yeast Display Mutant mAbs and Fabs Against Human LAP-TGFβ Isoform 1 at PH7.4 and PH6.0

This Example describes a Biacore analysis to evaluate binding kinetics for select yeast display mutant mAbs and corresponding Fabs against human LAP-TGFβ isoform 1.

A Series S C1 sensor chip (Cytiva, catalog BR100535) was immobilized with low densities of high affinity Fab 54BLH on a Biacore T200 instrument with 1×HBS-EP+(Teknova, catalog H8022). This surface served as a monovalent capture surface for a low-density layer of heterodimer human LAP-TGFβ1 complex leaving the second binding site exposed for monovalent kinetic binding analysis with both mAbs and Fabs. Immobilized flow cell 1 served as the reference and flow cells 2, 3 and 4 were used to evaluate the binding interaction between human LAP-TGFβ1 and yeast display mutant mAbs and Fabs.

Kinetic binding interactions were assessed at 25° C. and performed in 1×HBS-EP+ with 0.1 mg/mL BSA (Jackson Immunoresearch, catalog 001-000-162) at either pH7.4 or pH6.0. Approximately 15-25 RU of human LAP-TGFβ1-Fc was captured to the Fab capture surface followed by injection of 1:3 serially diluted Fabs and mAbs from 6 nM to 0.07 nM (Fabs: 54BLH, 55BLH, 60BLH, 63BLH, 64BLH-mAbs: 69BLH, 70BLH) and 1:3 serially diluted Fab and mAbs from 60 nM to 0.74 nM (Fabs: 59BLH, 66BLH, 67BLH—mAbs: 74BLH, 75BLH, 78BLH, 79BLH, 81BLH, 82BLH) including 0 nM. The binding data was double referenced by subtraction of signal from a reference flow cell and the 0 nM injections for evaluation of Fabs and mAbs respectively. Binding rate constants were calculated/determined by fitting the data with a 1:1 binding model (Cytiva Biacore T200 Evaluation software 3.0).

As shown in Table 55 and Table 56, in general the mutant yeast mAbs exhibited similar to slightly higher binding affinity to human LAP-TGFβ1 compared to the corresponding Fabs in both pH7.4 or pH6.0 with one exception, antibody 82BLH, which exhibited a 5 to 13-fold strengthened affinity as an mAb. Binding affinities at pH6.0 were reduced, -3 to 19-fold when compared to pH 7.4.

TABLE 55
Binding parameters comparison between yeast display mutant Fabs and mAbs binding in pH 7.4

| | | | | | | | | | Fab/mAb |
|---|---|---|---|---|---|---|---|---|---|
| Fab | Sorting | | k_{off} | K_{D} | mAb | | k_{off} | K_{D} | K_{D} |
| ID | population | kₒₙ | (s⁻¹) | (M) | ID | kₒₙ | (s⁻¹) | (M) | Ratio |
| 54BLH | 12 hours sort | 7.03 × 10⁶ | 3.34 × 10⁻⁵ | 4.77 × 10⁻¹² | 69BLH | — | — | n.d. | ND |
| 55BLH | 12 hours sort | 8.75 × 10⁶ | 1.09 × 10⁻³ | 1.25 × 10⁻¹⁰ | 70BLH | 1.45 × 10⁷ | 6.71 × 10⁻⁴ | 4.70 × 10⁻¹¹ | 0.3 |
| 59BLH | 2 hour P1 | 1.31 × 10⁷ | 5.75 × 10⁻⁴ | 4.40 × 10⁻¹¹ | 74BLH | 1.47 × 10⁷ | 3.74 × 10⁻⁴ | 2.60 × 10⁻¹¹ | 1.7 |
| 60BLH | 2 hour P1 | 9.65 × 10⁶ | 3.48 × 10⁻⁴ | 3.60 × 10⁻¹¹ | 75BLH | 1.91 × 10⁷ | 2.12 × 10⁻⁴ | 1.11 × 10⁻¹¹ | 3.2 |
| 63BLH | 2 hour P2 | 9.61 × 10⁶ | 3.41 × 10⁻³ | 3.55 × 10⁻¹⁰ | 78BLH | 2.22 × 10⁷ | 1.96 × 10⁻³ | 8.76 × 10⁻¹¹ | 4.0 |
| 64BLH | 2 hour P2 | 1.53 × 10⁷ | 9.39 × 10⁻⁴ | 6.20 × 10⁻¹¹ | 79BLH | 1.44 × 10⁷ | 5.52 × 10⁻⁴ | 3.80 × 10⁻¹¹ | 1.6 |
| 66BLH | 2 hour P3 | 5.76 × 10⁶ | 1.81 × 10⁻² | 3.13 × 10⁻⁹ | 81BLH | 5.40 × 10⁶ | 5.18 × 10⁻³ | 9.59 × 10⁻¹⁰ | 3.3 |
| 67BLH | 2 hour P3 | 6.69 × 10⁶ | 1.55 × 10⁻² | 2.36 × 10⁻⁹ | 82BLH | 7.82 × 10⁷ | 1.40 × 10⁻² | 1.78 × 10⁻¹⁰ | 13.3 |

n.d.—not determined, off-rate was beyond instrument limitations
ND—no data/not calculated

TABLE 56
Binding parameters comparison between yeast display mutant Fabs and mAbs binding in pH 6.0

| | | | | | | | | | Fab/mAb |
|---|---|---|---|---|---|---|---|---|---|
| Fab | Sorting | kₒₙ | k_{off} | K_{D} | mAb | kₒₙ | k_{off} | K_{D} | K_{D} |
| ID | population | (M⁻¹s⁻¹) | (s⁻¹) | (M) | ID | (M⁻¹s⁻¹) | (s⁻¹) | (M) | Ratio |
| 54BLH | 12 hours sort | 4.65 × 10⁶ | 1.01 × 10⁻⁴ | 2.17 × 10⁻¹¹ | 69BLH | 5.43 × 10⁶ | 6.18 × 10⁻⁵ | 1.14 × 10⁻¹¹ | 1.9 |
| 55BLH | 12 hours sort | 7.39 × 10⁶ | 2.48 × 10⁻³ | 3.35 × 10⁻¹⁰ | 70BLH | 8.47 × 10⁶ | 1.31 × 10⁻³ | 1.55 × 10⁻¹⁰ | 2.2 |
| 59BLH | 2 hour P1 | 7.08 × 10⁶ | 1.26 × 10⁻³ | 1.79 × 10⁻¹⁰ | 74BLH | 6.75 × 10⁶ | 8.92 × 10⁻⁴ | 1.33 × 10⁻¹⁰ | 1.3 |
| 60BLH | 2 hour P1 | 5.65 × 10⁶ | 1.67 × 10⁻³ | 2.96 × 10⁻¹⁰ | 75BLH | 6.86 × 10⁶ | 9.36 × 10⁻⁴ | 1.36 × 10⁻¹⁰ | 2.2 |
| 63BLH | 2 hour P2 | 7.51 × 10⁶ | 7.96 × 10⁻³ | 1.06 × 10⁻⁹ | 78BLH | 1.21 × 10⁷ | 3.99 × 10⁻³ | 3.31 × 10⁻¹⁰ | 3.2 |
| 64BLH | 2 hour P2 | 1.07 × 10⁷ | 1.94 × 10⁻³ | 1.82 × 10⁻¹⁰ | 79BLH | 1.12 × 10⁷ | 1.22 × 10⁻³ | 1.10 × 10⁻¹⁰ | 1.6 |
| 66BLH | 2 hour P3 | 1.96 × 10⁶ | 3.29 × 10⁻² | 1.68 × 10⁻⁸ | 81BLH | 5.65 × 10⁶ | 4.89 × 10⁻² | 9.11 × 10⁻⁹ | 1.8 |
| 67BLH | 2 hour P3 | 2.44 × 10⁶ | 4.60 × 10⁻² | 1.88 × 10⁻⁸ | 82BLH | 1.07 × 10⁷ | 3.65 × 10⁻² | 3.43 × 10⁻⁹ | 5.5 |

### Example 24. Epitope Competition Analysis of Yeast Display Mutants 74BLH and 81BLH Binding to Human LAP-TGFβ Isoform 1

This Example describes epitope competition analysis of yeast display mutant antibodies 74BLH and 81BLH on human LAP-TGFβ isoform 1 using Octet HTX instrument.

The epitope binning experiment was carried out via in-tandem format where 5 nM biotinylated human LAP-TGFβ1 was stably bound to the surface of streptavidin sensors (Sartorius, catalog 18-5021). The sensors were bound with primary antibody (mAb1) for 180 seconds at either 250 nM (74BLH) or 1000 nM (81BLH), to ensure full epitope saturation of human LAP-TGFβ1 followed by exposure to the secondary antibody (mAb2) for 180 seconds at either 250 nM (74BLH) or 1000 nM (81BLH). Neither antibody exhibited binding as mAB2 to human LAP-TGFβ1 when human LAP-TGFβ1 was pre-bound with that same antibody indicating fully saturated human LAP-TGFβ1. Antibodies were considered non-competitors of each other if they did not block each other's epitope on human LAP-TGFβ1 as demonstrated by >0.1 nm response for mAb2. Similarly, antibodies were considered competitors of each other if they blocked each other's epitope on human LAP-TGFβ1 as demonstrated by <0.1 nm response for mAb2.

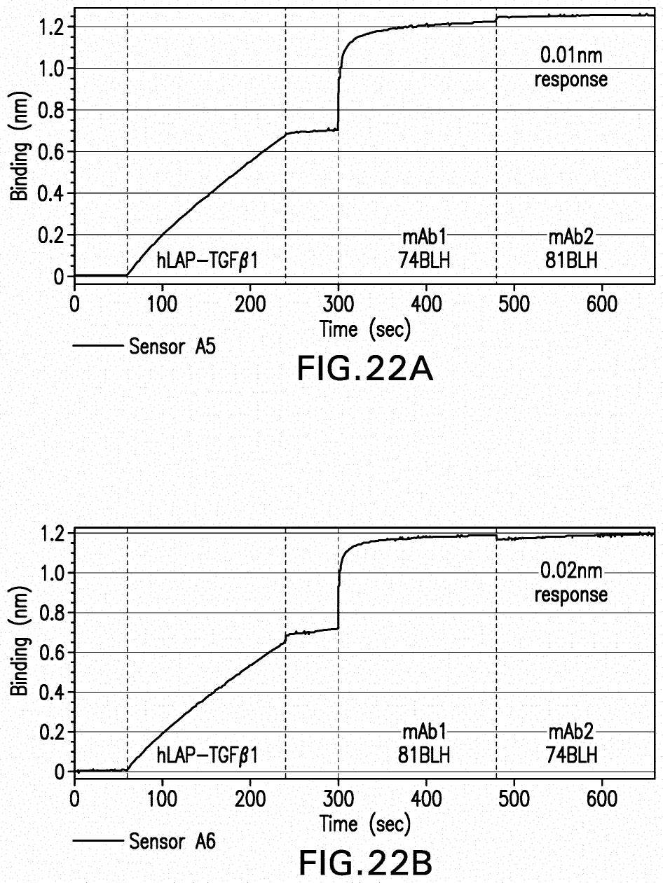

As shown in FIG. 22A and FIG. 22B, yeast display mutants 74BLH and 81BLH exhibit competitive binding on human LAP-TGFβ1 since neither antibody exhibits >0.1 nm response as mAb2 when human LAP-TGFβ1 is pre-saturated with the alternate antibody as mAb1.

### Example 25. Cryo-Em Structural Analysis of Humanized Antibody 015BLM with LAP-TGFβ1

The structure of humanized affinity matured 015BLM Fab in complex with human LAP-TGFβ1 was determined by cryo-EM to identify the epitope on LAP-TGFβ1 to which the antibody binds, and the paratope of humanized 015BLM1-Fab.

### Sample and Grids Preparation.

Humanized 015BLM mAb (which has heavy and light chain variable region sequences of SEQ ID NOs: 2598 and 2613, respectively) and Fab, and GARP-LAP-TGFβ1 were generated as described in Examples above. GARP-LAP-TGFβ1 was supplied in buffer A (25 mM Tris, 150 mM NaCl, pH 8) and the 015BLM Fab was supplied in buffer B (20 mM sodium acetate, 9% sucrose, pH 5.5). The sample used for cryo-EM experiments was prepared by mixing 5 μl of GARP-LAP-TGFβ1 (16.7 μM) and 5 μl of 015BLM Fab (8.4 μM), incubated at room temperature for 5 minutes, then mixed with 10 μl of buffer A for a final concentration of 8.4 μM for GARP-LAP-TGFβ1 and 4.2 μM for the 015BLM Fab.

Grids (UltrAufoil 1.2/1.3, 300 mesh) were prepared using a Vitrobot mark IV device using standard procedures. Grids were glow discharged using a GloQube unit (Quorum Technologies) with the factory suggested values (0.1 mbar, 35 mA for 60 seconds). The Vitrobot device was set with a chamber humidity of 100%; a chamber temperature of 4° C.; a blot time of 2.5 sec; a wait time of 0 sec; a blot force of 10. A volume (3 μl) of sample were applied to the grid, blotted, and then plunged into a liquid ethane bath; the frozen grid was then transferred to liquid nitrogen (LN2) and kept at LN2 temperature for all subsequent steps (clipping, transferring to the microscope cassette, and data collection).

### Data Collection and Structure Determination.

The data set was collected on a Titan Krios microscope equipped with a K3 detector. Data collection was done using the EPU software. 4832 movies were collected at a nominal magnification of 81,000× in super-resolution counting mode; the defocus range was set to be between −1 and −3 μm. The physical detector pixel size was 1.086 Å and the total dose was 44.57 e⁻/Å².

### Data Processing and Map Reconstruction.

The entire data processing and map reconstruction was carried out with cryoSPARC software system. The initial particle picking identified 3.5 million (M) particles. After a number of 2D classification jobs, about 1.9 million (M) particles were used to calculate an initial map (nominal resolution 4.9 Ang). The particle stack was further cleaned up after CTF refinement and local motion correction using 3D classifications, and the resulting set of particles (836K particles) were used to generate a map that after a NU-refinement had a nominal resolution of 3.42 Ang. Local (masked) refinement was then used to improve the resolution at the epitope-paratope interface. The resulting map of the local refinement (after density subtraction) had a 3.3 Å overall resolution, in which the details at the interface were greatly improved. The final density map was then filtered based on local resolution estimates, ranging from 2.75 to 4.43 Å and then used to build the model.

### Model Building and Refinement.

All model building and refinement were carried out using Collaborative Computational Project for electron cryo-microscopy (CCPEM) software suite and Crystallographic Object-Oriented Toolkit (COOT). The complex between LAP-TGFβ1 and humanized 20E6 Fab was used as the starting model and was initially positioned into the map as rigid bodies using Chimera interactive molecular graphics program, and the density was used to truncate the model and assign the humanized 20E6 Fab sequences. The macromolecular crystallographic refinement program REFMAC refinement module with Jelly body restraints and manual curation in COOT was carried out to optimize the model geometry. Table 57 summarizes the model refinement and statistics:

TABLE 57
Model refinement and statistics

| Symmetry Imposed | C1 |
|---|---|
| Particle used | 836,182 |
| Map resolution (Å) | 3.32 |
| FSC threshold | 0.143 |
| Map Resolution Range (Å) | 2.75-4.43 |

Refinement

| | |
|---|---|
| Map sharpening B-factor (Å2) | −139 |

Model composition

| | |
|---|---|
| Non hydrogen Atoms | 8361 |
| Protein residues | 533 |

| | |
|---|---|
| rmsd (bonds) (Å): | 0.0026 |
| rmsd (angles) (º): | 0.9423 |
| All-atom clashscore | 5.74 |

Ramachandran plot:

| | |
|---|---|
| outliers: | 0.20% |
| allowed: | 7.42% |
| favored: | 92.38% |

| | |
|---|---|
| Rotamer outliers: | 4.35% |

The final model contained two chains for the LAP-TGFβ1 dimer (chain A residues 1-61+70-83+102-193+216-241+293-331 and chain B residues 263-297+325-361; numbering for antigen assumes absence of signal peptide, and one molecule (heavy chain (VH), residues 1-117 and light chain (VL), residues 2-106) for the 15BLM Fab. One sugar moiety NAG was modeled at one of the glycosylation sites (chain A residue 53). Table 58 summarizes the epitope and paratope of LAP-TGFβ1 and 15BLM-Fab.

TABLE 58
Epitope and paratope of LAP-TGFβ1 and 15BLM-Fab*

| VH | TGFβ1 | VL |
|---|---|---|
| | Arg274 | Tyr30 |
| | Gly278 | Tyr30 |
| | Trp279 | Tyr50 |
| | Lys280 | Asp92 |
| Tyr101 | Val341 | |
| Tyr101 | Gly342 | |
| Asp33, Arg50, Asp52, Ser55 | Arg343 | |
| Tyr101 | Lys344 | |
| VH | LAP | VL |
| | Leu25 | Arg53 |
| Tyr104 | Ala31 | Tyr49, Arg53 |
| Tyr101, Gly102, Tyr104 | Ser32 | Tyr49, Tyr50, Arg53 |
| Gly102 | Pro33 | Tyr32, Tyr50, Arg53 |
| Gly102 | Pro34 | Tyr32 |
| Trp99, Gly102, Gly103 | Ser35 | Tyr32, Gly91 |
| | Gln36 | Asp92 |
| | Gly37 | Asp92, Leu94, Trp96 |
| His35, Arg50, Lys59 | Glu38 | Leu94, Trp96 |

*Residues from 15BLM-Fab VH or VL that interact with LAP-TGFβ1 residues are indicated. Hydrogen bonding interactions are indicated in bold (interaction cut-off set to 4.5Å; hydrogen bond interactions cut-off set to 3.5 Å).

### Equivalents:

Those skilled in the art will recognize, or be able to ascertain using no more than routine experimentation, many equivalents of the specific embodiments disclosed herein. Such equivalents are intended to be encompassed by the following claims.

1. An isolated antibody or antigen binding fragment thereof which specifically binds to LAP comprising: a heavy chain variable region comprising CDR1, CDR2, and CDR3 regions comprising the amino acid sequences of SEQ ID NOs: 2218, 2219, and 2220, respectively, and/or a light chain variable region comprising CDR1, CDR2, and CDR3 regions comprising the amino acid sequences of SEQ ID NOs: 2222, 2223, and 2224, respectively.

2. An isolated antibody or antigen binding fragment thereof which specifically binds to LAP comprising:
(a) a heavy chain variable region comprising CDR1, CDR2, and CDR3 regions comprising the amino acid sequences of SEQ ID NOs: 1285, 1286, and 1287, respectively, and/or a light chain variable region comprising CDR1, CDR2, and CDR3 regions comprising the amino acid sequences of SEQ ID NOs: 1289, 1290, and 1291, respectively;
(b) a heavy chain variable region comprising CDR1, CDR2, and CDR3 regions comprising the amino acid sequences of SEQ ID NOs: 1277, 1278, and 1279, respectively, and/or a light chain variable region comprising CDR1, CDR2, and CDR3 regions comprising the amino acid sequences of SEQ ID NOs: 1281, 1282, and 1283, respectively;
(c) a heavy chain variable region comprising CDR1, CDR2, and CDR3 regions comprising the amino acid sequences of SEQ ID NOs: 1269, 1270, and 1271, respectively, and/or a light chain variable region comprising CDR1, CDR2, and CDR3 regions comprising the amino acid sequences of SEQ ID NOs: 1273, 1274, and 1275, respectively;
(d) a heavy chain variable region comprising CDR1, CDR2, and CDR3 regions comprising the amino acid sequences of SEQ ID NOs: 1261, 1262, and 1263, respectively, and/or a light chain variable region comprising CDR1, CDR2, and CDR3 regions comprising the amino acid sequences of SEQ ID NOs: 1265, 1266, and 1267, respectively;
(e) a heavy chain variable region comprising CDR1, CDR2, and CDR3 regions comprising the amino acid sequences of SEQ ID NOs: 1253, 1254, and 1255, respectively, and/or a light chain variable region comprising CDR1, CDR2, and CDR3 regions comprising the amino acid sequences of SEQ ID NOs: 1257, 1258, and 1259, respectively;
(f) a heavy chain variable region comprising CDR1, CDR2, and CDR3 regions comprising the amino acid sequences of SEQ ID NOs: 1245, 1246, and 1247, respectively, and/or a light chain variable region comprising CDR1, CDR2, and CDR3 regions comprising the amino acid sequences of SEQ ID NOs: 1249, 1250, and 1251, respectively;
(g) a heavy chain variable region comprising CDR1, CDR2, and CDR3 regions comprising the amino acid sequences of SEQ ID NOs: 1237, 1238, and 1239, respectively, and/or a light chain variable region comprising CDR1, CDR2, and CDR3 regions comprising the amino acid sequences of SEQ ID NOs: 1241, 1242, and 1243, respectively
(h) a heavy chain variable region comprising CDR1, CDR2, and CDR3 regions comprising the amino acid sequences of SEQ ID NOs: 1229, 1230, and 1231, respectively, and/or a light chain variable region comprising CDR1, CDR2, and CDR3 regions comprising the amino acid sequences of SEQ ID NOs: 1233, 1234, and 1235, respectively;
(i) a heavy chain variable region comprising CDR1, CDR2, and CDR3 regions comprising the amino acid sequences of SEQ ID NOs: 1221, 1222, and 1223, respectively, and/or a light chain variable region comprising CDR1, CDR2, and CDR3 regions comprising the amino acid sequences of SEQ ID NOs: 1225, 1226, and 1227, respectively;
(j) a heavy chain variable region comprising CDR1, CDR2, and CDR3 regions comprising the amino acid sequences of SEQ ID NOs: 1213, 1214, and 1215, respectively, and/or a light chain variable region comprising CDR1, CDR2, and CDR3 regions comprising the amino acid sequences of SEQ ID NOs: 1217, 1218, and 1219, respectively;
(k) a heavy chain variable region comprising CDR1, CDR2, and CDR3 regions comprising the amino acid sequences of SEQ ID NOs: 95, 96, and 97, respectively, and/or a light chain variable region comprising CDR1, CDR2, and CDR3 regions comprising the amino acid sequences of SEQ ID NOs: 100, 101, and 102, respectively;
(1) a heavy chain variable region comprising CDR1, CDR2, and CDR3 regions comprising the amino acid sequences of SEQ ID NOs: 105, 106, and 107, respectively, and/or a light chain variable region comprising CDR1, CDR2, and CDR3 regions comprising the amino acid sequences of SEQ ID NOs: 110, 111, and 112, respectively;
(m) a heavy chain variable region comprising CDR1, CDR2, and CDR3 regions comprising the amino acid sequences of SEQ ID NOs: 115, 116, and 117, respectively, and/or a light chain variable region comprising CDR1, CDR2, and CDR3 regions comprising the amino acid sequences of SEQ ID NOs: 120, 121, and 122, respectively;
(n) a heavy chain variable region comprising CDR1, CDR2, and CDR3 regions comprising the amino acid sequences of SEQ ID NOs: 125, 126, and 127, respectively, and/or a light chain variable region comprising CDR1, CDR2, and CDR3 regions comprising the amino acid sequences of SEQ ID NOs:130, 131, and 132, respectively;
(o) a heavy chain variable region comprising CDR1, CDR2, and CDR3 regions comprising the amino acid sequences of SEQ ID NOs: 135, 136, and 137, respectively, and/or a light chain variable region comprising CDR1, CDR2, and CDR3 regions comprising the amino acid sequences of SEQ ID NOs: 140, 141, and 142, respectively;
(p) a heavy chain variable region comprising CDR1, CDR2, and CDR3 regions comprising the amino acid sequences of SEQ ID NOs: 145, 146, and 147, respectively, and/or a light chain variable region comprising CDR1, CDR2, and CDR3 regions comprising the amino acid sequences of SEQ ID NOs: 150, 151, and 152, respectively;
(q) a heavy chain variable region comprising CDR1, CDR2, and CDR3 regions comprising the amino acid sequences of SEQ ID NOs: 155, 156, and 157, respectively, and/or a light chain variable region comprising CDR1, CDR2, and CDR3 regions comprising the amino acid sequences of SEQ ID NOs: 160, 161, and 162, respectively;
(r) a heavy chain variable region comprising CDR1, CDR2, and CDR3 regions comprising the amino acid sequences of SEQ ID NOs: 165, 166, and 167, respectively, and/or a light chain variable region comprising CDR1, CDR2, and CDR3 regions comprising the amino acid sequences of SEQ ID NOs: 170, 171, and 172, respectively;
(s) a heavy chain variable region comprising CDR1, CDR2, and CDR3 regions comprising the amino acid sequences of SEQ ID NOs: 175, 176, and 177, respectively, and/or a light chain variable region comprising CDR1, CDR2, and CDR3 regions comprising the amino acid sequences of SEQ ID NOs: 180, 181, and 182, respectively;
(t) a heavy chain variable region comprising CDR1, CDR2, and CDR3 regions comprising the amino acid sequences of SEQ ID NOs: 185, 186, and 187, respectively, and/or a light chain variable region comprising CDR1, CDR2, and CDR3 regions comprising the amino acid sequences of SEQ ID NOs: 190, 191, and 192, respectively;
(u) a heavy chain variable region comprising CDR1, CDR2, and CDR3 regions comprising the amino acid sequences of SEQ ID NOs: 195, 196, and 197, respectively, and/or a light chain variable region comprising CDR1, CDR2, and CDR3 regions comprising the amino acid sequences of SEQ ID NOs: 200, 201, and 202, respectively;
(v) a heavy chain variable region comprising CDR1, CDR2, and CDR3 regions comprising the amino acid sequences of SEQ ID NOs: 205, 206, and 207, respectively, and/or a light chain variable region comprising CDR1, CDR2, and CDR3 regions comprising the amino acid sequences of SEQ ID NOs: 210, 211, and 212, respectively;
(w) a heavy chain variable region comprising CDR1, CDR2, and CDR3 regions comprising the amino acid sequences of SEQ ID NOs: 215, 216, and 217, respectively, and/or a light chain variable region comprising CDR1, CDR2, and CDR3 regions comprising the amino acid sequences of SEQ ID NOs: 220, 221, and 222, respectively;
(x) a heavy chain variable region comprising CDR1, CDR2, and CDR3 regions comprising the amino acid sequences of SEQ ID NOs: 225, 226, and 227, respectively, and/or a light chain variable region comprising CDR1, CDR2, and CDR3 regions comprising the amino acid sequences of SEQ ID NOs: 230, 231, and 232, respectively;
(y) a heavy chain variable region comprising CDR1, CDR2, and CDR3 regions comprising the amino acid sequences of SEQ ID NOs: 235, 236, and 237, respectively, and/or a light chain variable region comprising CDR1, CDR2, and CDR3 regions comprising the amino acid sequences of SEQ ID NOs: 240, 241, and 242, respectively;
(z) a heavy chain variable region comprising CDR1, CDR2, and CDR3 regions comprising the amino acid sequences of SEQ ID NOs: 245, 246, and 247, respectively, and/or a light chain variable region comprising CDR1, CDR2, and CDR3 regions comprising the amino acid sequences of SEQ ID NOs: 250, 251, and 252, respectively;
(aa) a heavy chain variable region comprising CDR1, CDR2, and CDR3 regions comprising the amino acid sequences of SEQ ID NOs: 255, 256, and 257, respectively, and/or a light chain variable region comprising CDR1, CDR2, and CDR3 regions comprising the amino acid sequences of SEQ ID NOs: 260, 261, and 262, respectively;
(bb) a heavy chain variable region comprising CDR1, CDR2, and CDR3 regions comprising the amino acid sequences of SEQ ID NOs: 265, 266, and 267, respectively, and/or a light chain variable region comprising CDR1, CDR2, and CDR3 regions comprising the amino acid sequences of SEQ ID NOs: 270, 271, and 272, respectively;
(cc) a heavy chain variable region comprising CDR1, CDR2, and CDR3 regions comprising the amino acid sequences of SEQ ID NOs: 275, 276, and 277, respectively, and/or a light chain variable region comprising CDR1, CDR2, and CDR3 regions comprising the amino acid sequences of SEQ ID NOs: 280, 281, and 282, respectively;
(dd) a heavy chain variable region comprising CDR1, CDR2, and CDR3 regions comprising the amino acid sequences of SEQ ID NOs: 285, 286, and 287, respectively, and/or a light chain variable region comprising CDR1, CDR2, and CDR3 regions comprising the amino acid sequences of SEQ ID NOs: 290, 291, and 292, respectively;
(ee) a heavy chain variable region comprising CDR1, CDR2, and CDR3 regions comprising the amino acid sequences of SEQ ID NOs: 295, 296, and 297, respectively, and/or a light chain variable region comprising CDR1, CDR2, and CDR3 regions comprising the amino acid sequences of SEQ ID NOs: 300, 301, and 302, respectively;
(ff) a heavy chain variable region comprising CDR1, CDR2, and CDR3 regions comprising the amino acid sequences of SEQ ID NOs: 305, 306, and 307, respectively, and/or a light chain variable region comprising CDR1, CDR2, and CDR3 regions comprising the amino acid sequences of SEQ ID NOs: 310, 311, and 312, respectively;
(gg) a heavy chain variable region comprising CDR1, CDR2, and CDR3 regions comprising the amino acid sequences of SEQ ID NOs: 315, 316, and 317, respectively, and/or a light chain variable region comprising CDR1, CDR2, and CDR3 regions comprising the amino acid sequences of SEQ ID NOs: 320, 321, and 322, respectively;
(hh) a heavy chain variable region comprising CDR1, CDR2, and CDR3 regions comprising the amino acid sequences of SEQ ID NOs: 325, 326, and 327, respectively, and/or a light chain variable region comprising CDR1, CDR2, and CDR3 regions comprising the amino acid sequences of SEQ ID NOs: 330, 331, and 332, respectively;
(ii) a heavy chain variable region comprising CDR1, CDR2, and CDR3 regions comprising the amino acid sequences of SEQ ID NOs: 335, 336, and 337, respectively, and/or a light chain variable region comprising CDR1, CDR2, and CDR3 regions comprising the amino acid sequences of SEQ ID NOs: 340, 341, and 342, respectively;
(jj) a heavy chain variable region comprising CDR1, CDR2, and CDR3 regions comprising the amino acid sequences of SEQ ID NOs: 345, 346, and 347, respectively, and/or a light chain variable region comprising CDR1, CDR2, and CDR3 regions comprising the amino acid sequences of SEQ ID NOs: 350, 351, and 352, respectively;
(kk) a heavy chain variable region comprising CDR1, CDR2, and CDR3 regions comprising the amino acid sequences of SEQ ID NOs: 355, 356, and 357, respectively, and/or a light chain variable region comprising CDR1, CDR2, and CDR3 regions comprising the amino acid sequences of SEQ ID NOs: 360, 361, and 362, respectively;
(ll) a heavy chain variable region comprising CDR1, CDR2, and CDR3 regions comprising the amino acid sequences of SEQ ID NOs: 365, 366, and 367, respectively, and/or a light chain variable region comprising CDR1, CDR2, and CDR3 regions comprising the amino acid sequences of SEQ ID NOs: 370, 371, and 372, respectively;
(mm) a heavy chain variable region comprising CDR1, CDR2, and CDR3 regions comprising the amino acid sequences of SEQ ID NOs: 375, 376, and 377, respectively, and/or a light chain variable region comprising CDR1, CDR2, and CDR3 regions comprising the amino acid sequences of SEQ ID NOs: 380, 381, and 382, respectively;
(nn) a heavy chain variable region comprising CDR1, CDR2, and CDR3 regions comprising the amino acid sequences of SEQ ID NOs: 385, 386, and 387, respectively, and/or a light chain variable region comprising CDR1, CDR2, and CDR3 regions comprising the amino acid sequences of SEQ ID NOs: 390, 391, and 392, respectively;
(oo) a heavy chain variable region comprising CDR1, CDR2, and CDR3 regions comprising the amino acid sequences of SEQ ID NOs: 395, 396, and 397, respectively, and/or a light chain variable region comprising CDR1, CDR2, and CDR3 regions comprising the amino acid sequences of SEQ ID NOs: 400, 401, and 402, respectively;
(pp) a heavy chain variable region comprising CDR1, CDR2, and CDR3 regions comprising the amino acid sequences of SEQ ID NOs: 405, 406, and 407, respectively, and/or a light chain variable region comprising CDR1, CDR2, and CDR3 regions comprising the amino acid sequences of SEQ ID NOs: 410, 411, and 412, respectively;
(qq) a heavy chain variable region comprising CDR1, CDR2, and CDR3 regions comprising the amino acid sequences of SEQ ID NOs: 415, 416, and 417, respectively, and/or a light chain variable region comprising CDR1, CDR2, and CDR3 regions comprising the amino acid sequences of SEQ ID NOs: 420, 421, and 422, respectively;
(rr) a heavy chain variable region comprising CDR1, CDR2, and CDR3 regions comprising the amino acid sequences of SEQ ID NOs: 425, 426, and 427, respectively, and/or a light chain variable region comprising CDR1, CDR2, and CDR3 regions comprising the amino acid sequences of SEQ ID NOs: 430, 431, and 432, respectively;
(ss) a heavy chain variable region comprising CDR1, CDR2, and CDR3 regions comprising the amino acid sequences of SEQ ID NOs: 435, 436, and 437, respectively, and/or a light chain variable region comprising CDR1, CDR2, and CDR3 regions comprising the amino acid sequences of SEQ ID NOs: 440, 441, and 442, respectively;
(tt) a heavy chain variable region comprising CDR1, CDR2, and CDR3 regions comprising the amino acid sequences of SEQ ID NOs: 455, 456, and 457, respectively, and/or a light chain variable region comprising CDR1, CDR2, and CDR3 regions comprising the amino acid sequences of SEQ ID NOs: 460, 461, and 462, respectively;
(uu) a heavy chain variable region comprising CDR1, CDR2, and CDR3 regions comprising the amino acid sequences of SEQ ID NOs: 465, 466, and 467, respectively, and/or a light chain variable region comprising CDR1, CDR2, and CDR3 regions comprising the amino acid sequences of SEQ ID NOs: 470, 471, and 472, respectively;
(vv) a heavy chain variable region comprising CDR1, CDR2, and CDR3 regions comprising the amino acid sequences of SEQ ID NOs: 475, 476, and 477, respectively, and/or a light chain variable region comprising CDR1, CDR2, and CDR3 regions comprising the amino acid sequences of SEQ ID NOs: 480, 481, and 482, respectively;
(ww) a heavy chain variable region comprising CDR1, CDR2, and CDR3 regions comprising the amino acid sequences of SEQ ID NOs: 485, 486, and 487, respectively, and/or a light chain variable region comprising CDR1, CDR2, and CDR3 regions comprising the amino acid sequences of SEQ ID NOs: 490, 491, and 492, respectively;
(xx) a heavy chain variable region comprising CDR1, CDR2, and CDR3 regions comprising the amino acid sequences of SEQ ID NOs: 495, 496, and 497, respectively, and/or a light chain variable region comprising CDR1, CDR2, and CDR3 regions comprising the amino acid sequences of SEQ ID NOs: 500, 501, and 502, respectively;
(yy) a heavy chain variable region comprising CDR1, CDR2, and CDR3 regions comprising the amino acid sequences of SEQ ID NOs: 505, 506, and 507, respectively, and/or a light chain variable region comprising CDR1, CDR2, and CDR3 regions comprising the amino acid sequences of SEQ ID NOs: 510, 511, and 512, respectively;
(zz) a heavy chain variable region comprising CDR1, CDR2, and CDR3 regions comprising the amino acid sequences of SEQ ID NOs: 515, 516, and 517, respectively, and/or a light chain variable region comprising CDR1, CDR2, and CDR3 regions comprising the amino acid sequences of SEQ ID NOs: 520, 521, and 522, respectively;
(aaa) a heavy chain variable region comprising CDR1, CDR2, and CDR3 regions comprising the amino acid sequences of SEQ ID NOs: 525, 526, and 527, respectively, and/or a light chain variable region comprising CDR1, CDR2, and CDR3 regions comprising the amino acid sequences of SEQ ID NOs: 530, 531, and 532, respectively;
(bbb) heavy chain variable region comprising CDR1, CDR2, and CDR3 regions comprising the amino acid sequences of SEQ ID NOs: 535, 536, and 537, respectively, and/or a light chain variable region comprising CDR1, CDR2, and CDR3 regions comprising the amino acid sequences of SEQ ID NOs: 540, 541, and 542, respectively;
(ccc) a heavy chain variable region comprising CDR1, CDR2, and CDR3 regions comprising the amino acid sequences of SEQ ID NOs: 545, 546, and 547, respectively, and/or a light chain variable region comprising CDR1, CDR2, and CDR3 regions comprising the amino acid sequences of SEQ ID NOs: 550, 551, and 552, respectively;
(ddd) a heavy chain variable region comprising CDR1, CDR2, and CDR3 regions comprising the amino acid sequences of SEQ ID NOs: 555, 556, and 557, respectively, and/or a light chain variable region comprising CDR1, CDR2, and CDR3 regions comprising the amino acid sequences of SEQ ID NOs: 560, 561, and 562, respectively;
(eee) a heavy chain variable region comprising CDR1, CDR2, and CDR3 regions comprising the amino acid sequences of SEQ ID NOs: 565, 566, and 567, respectively, and/or a light chain variable region comprising CDR1, CDR2, and CDR3 regions comprising the amino acid sequences of SEQ ID NOs: 570, 571, and 572, respectively;
(fff) a heavy chain variable region comprising CDR1, CDR2, and CDR3 regions comprising the amino acid sequences of SEQ ID NOs: 43, 44, and 45, respectively;
(ggg) a heavy chain variable region comprising CDR1, CDR2, and CDR3 regions comprising the amino acid sequences of SEQ ID NOs: 48, 49, and 50, respectively;
(hhh) a heavy chain variable region comprising CDR1, CDR2, and CDR3 regions comprising the amino acid sequences of SEQ ID NOs: 53, 54, and 55, respectively;
(iii) a heavy chain variable region comprising CDR1, CDR2, and CDR3 regions comprising the amino acid sequences of SEQ ID NOs: 58, 59, and 60, respectively;
(jjj) a heavy chain variable region comprising CDR1, CDR2, and CDR3 regions comprising the amino acid sequences of SEQ ID NOs: 63, 64, and 65, respectively;
(kkk) a heavy chain variable region comprising CDR1, CDR2, and CDR3 regions comprising the amino acid sequences of SEQ ID NOs: 68, 69, and 70, respectively;
(lll) a heavy chain variable region comprising CDR1, CDR2, and CDR3 regions comprising the amino acid sequences of SEQ ID NOs: 73, 74, and 75, respectively;
(mmm) a heavy chain variable region comprising CDR1, CDR2, and CDR3 regions comprising the amino acid sequences of SEQ ID NOs: 78, 79, and 80, respectively;
(nnn) a heavy chain variable region comprising CDR1, CDR2, and CDR3 regions comprising the amino acid sequences of SEQ ID NOs: 83, 84, and 85, respectively;
(ooo) a heavy chain variable region comprising CDR1, CDR2, and CDR3 regions comprising the amino acid sequences of SEQ ID NOs: 88, 89, and 90, respectively;
(ppp) a light chain variable region comprising CDR1, CDR2, and CDR3 regions comprising the amino acid sequences of SEQ ID NOs: 8, 9, and 10, respectively;
(qqq) a light chain variable region comprising CDR1, CDR2, and CDR3 regions comprising the amino acid sequences of SEQ ID NOs: 13, 14, and 15, respectively;
(rrr) a light chain variable region comprising CDR1, CDR2, and CDR3 regions comprising the amino acid sequences of SEQ ID NOs: 18, 19, and 20, respectively;
(sss) a light chain variable region comprising CDR1, CDR2, and CDR3 regions comprising the amino acid sequences of SEQ ID NOs: 23, 24, and 25, respectively;
(ttt) a light chain variable region comprising CDR1, CDR2, and CDR3 regions comprising the amino acid sequences of SEQ ID NOs: 28, 29, and 30, respectively;
(uuu) a light chain variable region comprising CDR1, CDR2, and CDR3 regions comprising the amino acid sequences of SEQ ID NOs: 33, 34, and 35, respectively;
(vvv) a light chain variable region comprising CDR1, CDR2, and CDR3 regions comprising the amino acid sequences of SEQ ID NOs: 38, 39, and 40, respectively; or
(zzz) a heavy chain variable region comprising CDR1, CDR2, and CDR3 regions comprising CDR1, CDR2, and CDR3 amino acid sequences selected from the group of sequences set forth in Table 41, Table 42, Table 43 and/or Table 45A, respectively, and/or a light chain variable region comprising CDR1, CDR2, and CDR3 regions comprising CDR1, CDR2, and CDR3 amino acid sequences selected from the group of sequences set forth in Table 41, Table 42, Table 43 and/or Table 45A, respectively.

3. The antibody or antigen binding fragment thereof of claim 1 which comprises a heavy chain variable region sequence comprising an amino acid sequence selected from the group consisting of SEQ ID NOs: 575-622, 661-685, 712-756, 794-827, 849-893; 921-950, 971-1009, 1037-1067, 1089-1113, 1138-1179, and 2589-2603; or a heavy chain variable region sequence which is at least 85%, 90%, 95%, 98%, or 99% identical to an amino acid sequence selected from the group consisting of SEQ ID NOs: 575-622, 661-685, 712-756, 794-827, 849-893: 921-950, 971-1009, 1037-1067, 1089-1113, 1138-1179, and 2589-2603.

4. (canceled)

5. The antibody or antigen binding fragment thereof of claim 1, which comprises a light chain variable region sequence selected from the group consisting of SEQ ID NOs: 623-660; 686-711, 757-793; 828-848, 894-920, 951-970, 1010-1036, 1068-1088, 1114-1137, and 1180-1211; or a light chain variable region sequence which is at least 85%, 90%, 95%, 98%, or 99% identical to an amino acid sequence selected from the group consisting of SEQ ID NOs: 623-660: 686-711, 757-793: 828-848, 894-920, 951-970, 1010-1036, 1068-1088, 1114-1137, and 1180-1211.

6. (canceled)

7. The antibody or antigen binding fragment thereof of claim 2 which comprises a heavy chain variable region sequence comprising an amino acid sequence selected from the group consisting of SEQ ID NOs: 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 458, 468, 478, 488, 498, 508, 518, 528, 538, 548, 558, and 568; or a heavy chain variable region sequence which is at least 85%, 90%, 95%, 98%, or 99% identical to the amino acid sequence selected from the group consisting of SEQ ID NOs: 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 458, 468, 478, 488, 498, 508, 518, 528, 538, 548, 558, and 568.

8. (canceled)

9. The antibody or antigen binding fragment thereof of claim 3, which comprises a light chain variable region sequence comprising the amino acid sequence selected from the group consisting of SEQ ID NOs: 103, 113, 123, 133, 143, 153, 163, 173, 183, 193, 203, 213, 223, 233, 243, 253, 263, 273, 283, 293, 303, 313, 323, 333, 343, 353, 363, 373, 383, 393, 403, 413, 423, 433, 443, 463, 473, 483, 493, 503, 513, 523, 533, 543, 553, 563, and 573; or a light chain variable region sequence which is at least 85%, 90%, 95%, 98%, or 99% identical to the amino acid sequence selected from the group consisting of SEQ ID NOs: 103, 113, 123, 133, 143, 153, 163, 173, 183, 193, 203, 213, 223, 233, 243, 253, 263, 273, 283, 293, 303, 313, 323, 333, 343, 353, 363, 373, 383, 393, 403, 413, 423, 433, 443, 463, 473, 483, 493, 503, 513, 523, 533, 543, 553, 563, and 573.

10-12. (canceled)

13. The antibody or antigen binding fragment thereof of claim 2 which comprises a heavy chain variable region sequence comprising an amino acid sequence selected from the group consisting of SEQ ID NOs: 46, 51, 56, 61, 66, 71, 76, 81, 86, and 91; or comprises a heavy chain variable region sequence which is at least 85%, 90%, 95%, 98%, or 99% identical to an amino acid sequence selected from the group consisting of SEQ ID NOs: 46, 51, 56, 61, 66, 71, 76, 81, 86, and 91.

14. The antibody or antigen binding fragment thereof of claim 2, which comprises a light chain variable region sequence comprising an amino acid sequence selected from the group consisting of SEQ ID NOs: 11, 16, 21, 26, 31, 36, and 41; or comprises a light chain variable region sequence which is at least 85%, 90%, 95%, 98%, or 99% identical to an amino acid sequence selected from the group consisting of SEQ ID NOs: 11, 16, 21, 26, 31, 36, and 41.

15. The antibody or antigen binding fragment thereof of claim 1 which comprises a heavy chain variable region sequence comprising the amino acid sequence selected from the group consisting of SEQ ID NO: 1212, 1220, 1228, 1236, 1244, 1252, 1260, 1268, 1276, 1284, 1295, 1305, 1315, 1325, 1335, 1345, 1355, 1365, 1375, and 2217; or comprises a heavy chain variable region sequence which is at least 85%, 90%, 95%, 98%, or 99% identical to the amino acid sequence selected from the group consisting of SEQ ID NO: 1212, 1220, 1228, 1236, 1244, 1252, 1260, 1268, 1276, 1284, 1295, 1305, 1315, 1325, 1335, 1345, 1355, 1365, 1375, and 2217.

16. The antibody or antigen binding fragment thereof of claim 1, which comprises a light chain variable region sequence comprising the amino acid sequence selected from the group consisting of SEQ ID NO: 1216, 1224, 1232, 1240, 1248, 1256, 1264, 1272, 1280, 1288, 1300, 1310, 1320, 1330, 1340, 1350, 1360, 1370, 1380, 2221; or comprises a light chain variable region sequence which is at least 85%, 90%, 95%, 98%, or 99% identical to the amino acid sequence selected from the group consisting of SEQ ID NO: 1216, 1224, 1232, 1240, 1248, 1256, 1264, 1272, 1280, 1288, 1300, 1310, 1320, 1330, 1340, 1350, 1360, 1370, 1380, 2221.

17-52. (canceled)

53. The antibody or antigen binding fragment thereof of claim 1 which comprises a heavy chain variable region sequence comprising an amino acid sequence described in Table 42, Table 43; or Table 45B or comprises a heavy chain variable region sequence which is at least 85%, 90%, 95%, 98%, or 99% identical to an amino acid sequence described in Table 42.

54. The antibody or antigen binding fragment thereof of claim 1, which comprises a light chain variable region sequence comprising an amino acid sequence described in Table 42, Table 43; or Table 45B or comprises a light chain variable region sequence which is at least 85%, 90%, 95%, 98%, or 99% identical to an amino acid sequence described in Table 42 or Table 43.

55. The antibody or antigen binding fragment thereof of claim 1 which comprises a heavy chain variable region sequence comprising the amino acid sequence selected from the group consisting of SEQ ID NO: 1385, 1395, 1405, 1415, 1425, 1435, 1445, 1455, 1465, 1475, 1485, 1495, 1505, 1515, 1525, 1535, 1545 and 1555; or comprises a heavy chain variable region sequence which is at least 85%, 90%, 95%, 98%, or 99% identical to the amino acid sequence selected from the group consisting of SEQ ID NO: 1385, 1395, 1405, 1415, 1425, 1435, 1445, 1455, 1465, 1475, 1485, 1495, 1505, 1515, 1525, 1535, 1545 and 1555.

56. The antibody or antigen binding fragment thereof of any one of claim 1, which comprises a light chain variable region sequence comprising the amino acid sequence selected from the group consisting of SEQ ID NO: 1390, 1400, 1410, 1420, 1430, 1440, 1450, 1460, 1470, 1480, 1490, 1500, 1510, 1520, 1530, 1540, and 1550; or comprises a light chain variable region sequence which is at least 85%, 90%, 95%, 98%, or 99% identical to the amino acid sequence selected from the group consisting of SEQ ID NO: 1390, 1400, 1410, 1420, 1430, 1440, 1450, 1460, 1470, 1480, 1490, 1500, 1510, 1520, 1530, 1540, and 1550.

57-91. (canceled)

92. The antibody or antigen binding fragment thereof of claim 1 which comprises heavy and light chain variable region sequences which are at least 85%, 90%, 95%, 98%, or 99% identical to the amino acid sequences selected from the group consisting of: (1) SEQ ID NOs: 1295 and 1300, respectively, (2) SEQ ID NOs: 1305 and 1310, respectively, (3) SEQ ID NOs: 1315 and 1320, respectively; (4) SEQ ID NOs: 1325 and 1330, respectively; (5) SEQ ID NOs: 1335 and 1340, respectively; (6) SEQ ID NOs: 1345 and 1350, respectively; (7) SEQ ID NOs: 1355 and 1360, respectively; (8) SEQ ID NOs: 1365 and 1370, respectively; (9) SEQ ID NOs: 1375 and 1380, respectively; (10) SEQ ID NOs: 1385 and 1390, respectively; (11) SEQ ID NOs: 1395 and 1400, respectively; (12) SEQ ID NOs: 1405 and 1410, respectively; (13) SEQ ID NOs: 1415 and 1420, respectively; (14) SEQ ID NOs: 1425 and 1430, respectively; (15) SEQ ID NOs: 1435 and 1440, respectively; (16) SEQ ID NOs: 1445 and 1450, respectively; (17) SEQ ID NOs: 1455 and 1460, respectively; (18) SEQ ID NOs: 1465 and 1470, respectively; (19) SEQ ID NOs: 1475 and 1480, respectively; (20) SEQ ID NOs: 1485 and 1490, respectively; (21) SEQ ID NOs: 1495 and 1500, respectively; (22) SEQ ID NOs: 1505 and 1510, respectively; (23) SEQ ID NOs: 1515 and 1520, respectively; (24) SEQ ID NOs: 1525 and 1530, respectively; (25) SEQ ID NOs: 1535 and 1540, respectively; (26) SEQ ID NOs: 1545 and 1550, respectively; and (27) SEQ ID NOs: 1555 and 1560, respectively.

93. (canceled)

94. The antibody or antigen binding fragment thereof of any of claim 53, which comprises heavy chain and light chain sequences which are at least 85%, 90%, 95%, 98%, or 99% identical to the amino acid sequences selected from the group consisting of: (1) SEQ ID NOs: 2589 and 2604; (2) SEQ ID NOs:2590 and 2605; (3) SEQ ID NOs: 2591 and 2606; (4) SEQ ID NOs:2592 and 2607; (5) SEQ ID NOs: 2593 and 2608; (6) SEQ ID NOs: 2594 and 2609; (7) SEQ ID NOs: 2595 and 2610; (8) SEQ ID NOs: 2596 and 2611; (9) SEQ ID NOs: 2597 and 2612; (10) SEQ ID NOs: 2598 and 2613; (11) SEQ ID NOs: 2599 and 2614; (12) SEQ ID NOs: 2600 and 2615; (13) SEQ ID NOs: 2601 and 2616; (14) SEQ ID NOs: 2602 and 2617; and (15) SEQ ID NOs: 2603 and 2618.

95. (canceled)

96. The antibody or antigen binding fragment thereof of any of claim 2, which comprises a heavy chain sequence which is at least 85%, 90%, 95%, 98%, or 99% identical to the amino acid sequences selected from the group consisting of: 47, 52, 57, 62, 67, 72, 77, 82, 87, and 92; and which comprises a light chain sequence which is at least 85%, 90%, 95%, 98%, or 99% identical to the amino acid sequences selected from the group consisting of: 12, 17, 22, 27, 32, 37, and 42.

97. (canceled)

98. The antibody or antigen binding fragment thereof of any of claim 2, which comprises heavy and light chain sequences which are at least 85%, 90%, 95%, 98%, or 99% identical to the amino acid sequences selected from the group consisting of: (1) SEQ ID NOs: 99 and 104, respectively, (2) SEQ ID NOs: 109 and 114, respectively, (3) SEQ ID NOs: 119 and 124, respectively; (4) SEQ ID NOs: 129 and 134, respectively; (5) SEQ ID NOs: 139 and 144, respectively; (6) SEQ ID NOs: 149 and 154, respectively; (7) SEQ ID NOs: 159 and 164, respectively; (8) SEQ ID NOs: 169 and 174, respectively; (9) SEQ ID NOs: 179 and 184, respectively; (10) SEQ ID NOs: 189 and 194, respectively; (11) SEQ ID NOs: 199 and 204, respectively; (12) SEQ ID NOs: 209 and 214, respectively; (13) SEQ ID NOs: 219 and 224, respectively; (14) SEQ ID NOs: 229 and 234, respectively; (15) SEQ ID NOs: 239 and 244, respectively; (16) SEQ ID NOs: 249 and 254, respectively; (17) SEQ ID NOs: 259 and 264, respectively; (18) SEQ ID NOs: 269 and 274, respectively; (19) SEQ ID NOs: 279 and 284, respectively; (20) SEQ ID NOs: 289 and 294, respectively; (21) SEQ ID NOs: 299 and 304, respectively; (22) SEQ ID NOs: 309 and 314, respectively; (23) SEQ ID NOs: 319 and 324, respectively; (24) SEQ ID NOs: 329 and 334, respectively; (25) SEQ ID NOs: 339 and 344, respectively; (26) SEQ ID NOs: 349 and 354, respectively; (27) SEQ ID NOs: 359 and 364, respectively; (28) SEQ ID NOs: 369 and 374, respectively; (29) SEQ ID NOs: 379 and 384, respectively; (30) SEQ ID NOs: 389 and 394, respectively; (31) SEQ ID NOs: 399 and 404, respectively; (32) SEQ ID NOs: 409 and 414, respectively; (33) SEQ ID NOs: 419 and 424, respectively; (34) SEQ ID NOs: 429 and 434, respectively; (35) SEQ ID NOs: 439 and 444, respectively; (36) SEQ ID NOs: 459 and 464, respectively; (37) SEQ ID NOs: 469 and 474, respectively; (38) SEQ ID NOs: 479 and 484, respectively; (39) SEQ ID NOs: 489 and 494, respectively; (40) SEQ ID NOs: 499 and 504, respectively; (43) SEQ ID NOs: 509 and 514, respectively; (42) SEQ ID NOs: 519 and 524, respectively; (43) SEQ ID NOs: 529 and 534, respectively; (44) SEQ ID NOs: 539 and 544, respectively; (45) SEQ ID NOs: 549 and 554, respectively; and (46) SEQ ID NOs: 559 and 564, respectively.

99-101. (canceled)

102. The antibody or antigen binding fragment thereof of claim 1, which comprises heavy and light chain variable region sequences which are at least 85%, 90%, 95%, 98%, or 99% identical to the amino acid sequences selected from the group consisting of: (1) SEQ ID NOs: 1296 and 1301, respectively, (2) SEQ ID NOs: 1306 and 1311, respectively, (3) SEQ ID NOs: 1316 and 1321, respectively; (4) SEQ ID NOs: 1326 and 1331, respectively; (5) SEQ ID NOs: 1336 and 1341, respectively; (6) SEQ ID NOs: 1346 and 1351, respectively; (7) SEQ ID NOs: 1356 and 1361, respectively; (8) SEQ ID NOs: 1366 and 1371, respectively; (9) SEQ ID NOs: 1376 and 1381, respectively; (10) SEQ ID NOs: 1386 and 1391, respectively; (11) SEQ ID NOs: 1396 and 1401, respectively; (12) SEQ ID NOs: 1406 and 1411, respectively; (13) SEQ ID NOs: 1416 and 1421, respectively; (14) SEQ ID NOs: 1426 and 1431, respectively; (15) SEQ ID NOs: 1436 and 1441, respectively; (16) SEQ ID NOs: 1446 and 1451, respectively; (17) SEQ ID NOs: 1456 and 1461, respectively; (18) SEQ ID NOs: 1466 and 1471, respectively; (19) SEQ ID NOs: 1476 and 1481, respectively; (20) SEQ ID NOs: 1486 and 1491, respectively; (21) SEQ ID NOs: 1496 and 1501, respectively; (22) SEQ ID NOs: 1506 and 1511, respectively; (23) SEQ ID NOs: 1516 and 1521, respectively; (24) SEQ ID NOs: 1526 and 1531, respectively; (25) SEQ ID NOs: 1536 and 1541, respectively; (26) SEQ ID NOs: 1546 and 1551, respectively; (27) SEQ ID NOs: 1556 and 1561, respectively.

103-106. (canceled)

107. The antibody or antigen binding fragment thereof of claim 2, which comprises heavy and light chain variable region sequences which are at least 85%, 90%, 95%, 98%, or 99% identical to the amino acid sequences selected from the group consisting of: SEQ ID NOs: 2619 and 2634, respectively, (2) SEQ ID NOs: 2620 and 2635, respectively, (3) SEQ ID NOs: 2621 and 2636, respectively; (4). SEQ ID NOs: 2622 and 2637; (5) SEQ ID NOs: 2623 and 2638; (6) SEQ ID NOs: 2624 and 2639; (7) SEQ ID NOs: 2625 and 2640; (8) SEQ ID NOs: 2626 and 2641; (9) SEQ ID NOs: 2627 and 2642; (10) SEQ ID NOs: 2628 and 2643; (11) SEQ ID NOs: 2629 and 2644; (12) SEQ ID NOs: 2630 and 2645; (13) SEQ ID NOs: 2631 and 2646; (14) SEQ ID NOs: 2632 and 2647; and (15) SEQ ID NOs: 2633 and 2648.

108-153. (canceled)
